(19) United States
(12) Patent Application Publication
(10) Pub. No.: US 2021/0138071 A1
(43) Pub. Date: May 13, 2021

(54) SILK-BASED PRODUCTS AND METHODS OF USE

(71) Applicant: Cocoon Biotech Inc., Mansfield, MA (US)

(72) Inventors: Michael Santos, Mansfield, MA (US); Scott Delisle, Mansfield, MA (US); Ailis Tweed-Kent, Mansfield, MA (US); Lindsey Easthon, Salem, MA (US); Bhushan S. Pattni, Canton, MA (US)

(21) Appl. No.: 16/762,547

(22) PCT Filed: Nov. 9, 2018
(86) PCT No.: PCT/US2018/059996

§ 371 Date: May 8, 2020

Related U.S. Application Data
(60) Provisional application No. 62/584,153, filed on Nov. 10, 2017.
(60) Provisional application No. 62/659,213, filed on Apr. 18, 2018.
(60) Provisional application No. 62/659,209, filed on Apr. 18, 2018.
(60) Provisional application No. 62/680,386, filed on Jun. 4, 2018.
(60) Provisional application No. 62/680,371, filed on Jun. 4, 2018.

(51) Int. Cl. A61K 47/42; A61K 31/415; C07K 16/22; A61K 38/47

(52) U.S. Cl. CPC A61K 47/42; A61K 38/47; C07K 16/22; A61K 31/415

(57) ABSTRACT

Embodiments of the present disclosure include silk-based products and related methods of use in a variety of applications. Included are applications in the fields of medicine, veterinary medicine, agriculture, and material science.

### CROSS REFERENCE TO RELATED APPLICATIONS

This application claims priority to 62/584,153 filed on Nov. 10, 2017 entitled Manufacture and Uses of Silk Fibroin, 62/659,213 filed Apr. 18, 2018 entitled Silk-Based Products and Methods of Use, 62/659,209 filed Apr. 18, 2018 entitled Ocular Silk-Based Products and Methods of Use, 62/680,386 filed Jun. 4, 2018 entitled Silk-Based Products and Methods of Use, and 62/680,371 filed Jun. 4, 2018 entitled Ocular Silk-Based Products and Methods of Use, the contents of each of which are herein incorporated by reference in their entirety.

### FIELD OF THE INVENTION

The present invention relates to formulations and methods of using silk in therapeutic, agricultural, and materials applications. Specifically provided are silk-based product formulations.

### BACKGROUND OF THE INVENTION

Silk is a naturally occurring polymer. Most silk fibers are derived from silkworm moth (Bombyx mori) cocoons and include silk fibroin and sericin proteins. Silk fibroin is a fibrous material that forms a polymeric matrix bonded together with sericin. In nature, silk is formed from a concentrated solution of these proteins that are extruded through silkworm spinnerets to produce a highly insoluble fiber. These fibers have been used for centuries to form threads used in garments and other textiles.

Many properties of silk make it an attractive candidate for products serving a variety of industries. Polymer strength and flexibility has supported classical uses of silk in textiles and materials, while silk biocompatibility has gained attention more recently for applications in the fields of medicine and agriculture. Additional uses for silk in applications related to material science are being explored as technologies for producing and processing silk advance.

Although a variety of products and uses related to silk are being developed, there remains a need for methods of producing and processing silk and silk-based products that can meet modern demands. Additionally, there remains a need for silk-based products that can leverage silk polymer strength, flexibility, biocompatibility, and other properties to meet needs in the fields of medicine, agriculture, and material sciences. The present disclosure addresses these needs by providing methods for producing and processing silk as well as silk-based products useful in a variety of industries.

### SUMMARY OF THE INVENTION

In some embodiments, the present disclosure provides a silk-based product (SBP) for use in a therapeutic application, an agricultural application, and/or a material science application, wherein the SBP includes processed silk that is derived from one or more of raw silk, silk fiber, silk fibroin, and a silk fibroin fragment. The SBP may be used in a therapeutic application, wherein the SBP includes or is combined with one or more of: (a) a pharmaceutical composition, the pharmaceutical composition optionally including one or more of: (i) an excipient, wherein the excipient includes one or more members including, but not limited to, any of those listed in Table 1; and (ii) a therapeutic agent, wherein the therapeutic agent includes one or more members such as, but not limited to, any of those listed in Table 3; (b) an implant, the implant optionally including one or more of: (i) an excipient, wherein the excipient includes one or more members such as, but not limited to, any of those listed in Table 1; (ii) a therapeutic agent, where the therapeutic agent includes one or more members such as, but not limited to, any of those listed in Table 3; (iii) a coating; (iv) a gel or hydrogel; (v) a scaffold; (vi) a particle; and (vii) a device, where the device includes one or more members such as, but not limited to, any of those listed in Table 6; (c) a coating, the coating optionally including one or more of: (i) an excipient, where the excipient includes one or more members such as, but not limited to, any of those listed in Table 1; and (ii) a therapeutic agent, where the therapeutic agent includes one or more members such as, but not limited to, any of those listed in Table 3; (d) a food or health supplement; and (e) a device, the device optionally including one or more of: (i) a synthetic material; and (ii) a therapeutic agent, wherein the therapeutic agent includes one or more members such as, but not limited to, any of those listed in Table 3.

In some embodiments, the present disclosure provides an SBP for use in an agricultural application, wherein the SBP includes or is combined with one or more members such as, but not limited to, (a) an agricultural composition, where the agricultural composition optionally includes one or more members such as, but not limited to, (i) a cargo, where the cargo includes one or more members such as, but not limited to, any of those listed in Table 7; (ii) a coating; (iii) a fertilizer; (iv) a nutrient, where the nutrient includes one or more members such as, but not limited to, any of those listed in Table 7; (v) an agricultural product; (vi) a pest control agent, where the pest control agent optionally includes a pesticide such as, but not limited to, (1) a parasiticide, where the parasiticide includes one or more members such as, but not limited to, any of those listed in Table 7; (2) an insecticide, where the insecticide includes one or more members such as, but not limited to, any of those listed in Table 7; (3) an herbicide, where the herbicide includes one or more members such as, but not limited to, any of those listed in Table 7; and (4) an anti-fungal or fungicide, where the anti-fungal or fungicide includes one or more members such as, but not limited to, any of those listed in Table 7; (vii) a soil stabilizer including one or more members such as, but not limited to, any of those listed in Table 7; (viii) a biological system including at least one microbe and/or probiotic; and (ix) an agricultural therapeutic agent including one or more members such as, but not limited to, any of those listed in Table 3 and any of those listed in Table 7; and (b) an agricultural device, where the agricultural device optionally includes one or more members such as, but not limited to, (i) an article of agricultural equipment; (ii) a crop storage device; (iii) a landscaping fabric; and (iv) a pest control device.

SBPs for use in a material science application, may include or be combined with a material, where the material includes one or more articles such as, but not limited to, (a) an adhesive; (b) a biomaterial; (c) a coating; (d) a conductor; (e) a composting agent; (f) a cosmetic, the cosmetic optionally including one or more members such as, but not limited to, any of those listed in Table 9; (g) an emulsifier; (h) an excipient, the excipient optionally including one or more members such as, but not limited to, any of those listed in Table 1; (i) a fiber; (j) a film (k) a filter; (l) a food product or additive; (m) an insulator; (n) a lubricant; (o) a membrane; (p) a metal or metal replacement; (q) a microneedle; (r) a nanomaterial; (s) a particle; (t) a paper additive; (u) a plastic or plastic replacement; (v) a polymer; (w) a sensor; (x) a textile; and (y) a thickening agent.

In some embodiments, the SBPs include processed silk that includes silk fibroin, where the silk fibroin includes a beta sheet, an alpha helix, a coiled coil, and/or a random coil. The silk fibroin may include a silk fibroin polymer, a silk fibroin monomer, and/or a silk fibroin fragment. The processed silk may include a silk fibroin fragment, where the silk fibroin fragment includes a silk fibroin heavy chain fragment and/or a silk fibroin light chain fragment. The silk fibroin may include a plurality of silk fibroin fragments. The plurality of silk fibroin fragments may include a molecular weight of from about 1 kDa to about 350 kDa. The plurality of silk fibroin fragments may be generated using a dissociation procedure. The dissociation procedure may include one or more members such as, but not limited to, heating, acid treatment, base treatment, chaotropic agent treatment, sonication, and electrolysis. The dissociation procedure may include heating, wherein raw silk, silk fiber, and/or silk fibroin are heated to a temperature of from about 30° C. to about 1,000° C. The raw silk, silk fiber, and/or silk fibroin may be heated for from about 1 second to about 24 hours. The processed silk may be harvested from a silk producer. The silk producer may be a wild type organism. The silk producer may be a genetically modified organism. The silk producer may be, but is not limited to, an insect or an arachnid. The silk producer may be, but is not limited to, Bombyx mandarina, Bombyx mori, Bombyx sinesis, Anaphe moloneyi, Anaphe panda, Anaphe reticulate, Anaphe ambrizia, Anaphe carteri, Anaphe venata, Anapha infracta, Antheraea assamensis, Antheraea assama, Antheraea mylitta, Antheraea pernyi, Antheraea yamamai, Antheraea polyphemus, Antheraea oculea, Anisota senatoria, Apis mellifera, Araneus diadematus, Araneus cavaticus, Automeris io, Atticus atlas, Copaxa multifenestrata, Coscinocera hercules, Callosamia promethea, Eupackardia calleta, Eurprosthenops australis, Gonometa postica, Gonometa rufobrunnea, Hyalophora cecropia, Hyalophora euryalus, Hyalophora gloveri, Miranda auretia, Nephila madagascarensis, Nephila clavipes, Pachypasa otus, Pachypasa atus, Philosamia ricini, Pinna squamosa, Rothschildia hesperis, Rothschildia lebeau, Samia cynthia, and Samia ricini. The insect may be Bombyx mori. The silk producer may be a genetically modified organism, wherein the genetically modified organism includes at least one nucleic acid encoding at least one silk protein. The at least one silk protein may include one or more members such as, but not limited to, a silk fibroin heavy chain, a silk fibroin light chain, a silk fibroin fragment, and sericin. The genetically modified organism may be such as, but not limited to, an insect, an arachnid, a bacteria, a yeast, a mammalian cell, and a plant cell. The processed silk may be derived from synthetic silk. The processed silk may include or be included in one or more members such as, but not limited to, yarn, thread, string, a nanofiber, a particle, a nanoparticle, a microsphere, a nanosphere, a powder, a solution, a gel, a hydrogel, an organogel, a mat, a film, a foam, a membrane, a rod, a tube, a patch, a sponge, a scaffold, a capsule, an excipient, an implant, a solid, a coating, and a graft. The SBP may include one or more formats selected from the group consisting of yarns, fibers, sheets, discs, nanofibers, particles, cylinders, nanoparticles, solutions, gels, hydrogels, organogels, powders, solids, threads, spuns, mats, films, foams, suspensions, sprays, membranes, rods, tubes, microspheres, nanospheres, cones, patches, sponges, scaffolds, capsules, nets, grafts, vapors, emulsions, tablets, and adhesives. The SBP may include one or more pores. The one or more pores may be formed naturally or during one or more processing steps. The one or more processing steps may include one or more of sonication, centrifugation, modulating silk fibroin concentration, modulating solute concentration, modulating excipient concentration, modulating pH, chemical modification, crosslinking, combining with cells, combining with bacteria, and combining with viral particles.

SBPs for use in therapeutic applications may include use in a therapeutic application such as, but not limited to, (a) treatment, prevention, mitigation, alleviation, and/or curing of a disease, disorder, and/or condition in a subject; (b) promotion of health, nutrition, and/or wellbeing in a subject; (c) support or promotion of reproduction in a subject; (d) preparation of a therapeutic device; and (e) diagnosis of a disease, disorder, and/or condition in a subject. The subject may be a human subject or a non-human animal subject. The SBP may be formulated or formatted for administration to the subject. The SBP may include a therapeutic agent, where the therapeutic agent includes a biological agent. The biological agent may include one or more members such as, but not limited to, a macromolecule, a carbohydrate, a peptide, a protein, a nucleic acid, a virus, a virus particle, a vesicle, a cell, a spore, a bacteria, and a tissue. The biological agent may include a protein, wherein the protein includes one or more members such as, but not limited to, any of those listed in Table 3. The biological agent may include a macromolecule, where the macromolecule includes one or more members such as, but not limited to, (a) a carbohydrate, where the carbohydrate includes one or more members such as, but not limited to, any of those listed in Table 3; (b) a lipid, where the lipid includes one or more members such as, but not limited to, any of those listed in Table 3; (c) a steroid, where the steroid includes one or more members such as, but not limited to, any of those listed in Table 3; (d) a nucleotide; (e) a peptide, wherein the peptide includes one or more members such as, but not limited to, any of those listed in Table 3; and (f) an amino acid. The biological agent may include a cell, where the cell may be, but not limited to, any of those listed in Table 3. The biological agent may include a nucleic acid, wherein the nucleic acid includes one or more members such as, but not limited to, RNA, DNA, cDNA, siRNA, dsRNA, RNAi, miRNA, shRNA, RNA-DNA duplex, RNA-RNA duplex. DNA duplex, an aptamer, and a plasmid. The biological agent may include a virus, wherein the virus may be, but is not limited to, an adenovirus and a lentivirus. The SBP may include therapeutic agent, where the therapeutic agent includes a small molecule. The therapeutic agent may include one or more members such as, but not limited to, (a) an analgesic agent, where the analgesic agent includes one or more members such as, but not limited to, any of those listed in Table 3; (b) an anesthetic agent; (c) an antianxiety medication; (d) an antibacterial agent, where the antibacterial agent includes one or more members such as, but not limited to, any of those listed in Table 3; (e) an antibody, where the antibody includes one or more members such as, but not limited to, any of those listed in Table 3; (f) an antidepressant; (g) an anti-emetic agent; (h) an antifungal agent, where the antifungal agent includes one or more members such as, but not limited to, any of those listed in Table 3; (i) an antigen, where the antigen includes one or more members such as, but not limited to, any of those listed in Table 3; (j) an anti-inflammatory agent, where the anti-inflammatory agent includes one or more members such as, but not limited to, any of those listed in Table 3; (k) an antimalarial agent, wherein the antimalarial agent includes one or more members such as, but not limited to, any of those listed in Table 3; (l) an antiparasitic agent; (m) an antipsychotic agent; (n) an antipyretic agent, where the antipyretic agent may be, but is not limited to, choline salicylate, magnesium salicylate, metamizole, nimesulide, phenazone, salicylate, and sodium salicylate; (o) an antiseptic agent, where the antiseptic agent includes one or more members such as, but not limited to, any of those listed in Table 3; (p) an antiviral agent; (q) a blood thinner; (r) a chemotherapeutic agent; (s) a contrasting agent; (t) a cytokine, where the cytokine includes one or more members such as, but not limited to, any of those listed in Table 3; (u) an herbal preparation, wherein the herbal preparation includes one or more members selected from the group consisting of any of those listed in Table 3; (v) a health supplement, where the health supplement includes one or more members such as, but not limited to, any of those listed in Table 3; (w) a hemostatic agent; (x) a hormone, where the hormone includes one or more members such as, but not limited to, any of those listed in Table 3; (y) an imaging agent; (z) an inhalant or respiratory agent; (aa) a motility or anti-motility agent; (bb) a non-steroidal anti-inflammatory drug (NSAID), where the NSAID includes one or more members such as, but not limited to, any of those listed in Table 3; (cc) an oxidant and/or antioxidant, wherein the oxidant and/or antioxidant includes one or more members such as, but not limited to, any of those listed in Table 3; (dd) a peptide, where the peptide includes one or more members such as, but not limited to, any of those listed in Table 3; (ee) a smoking cessative agent; (ff) a statin, where the statin includes one or more members such as, but not limited to, any of those listed in Table 3; (gg) a stimulant, where the stimulant includes one or more members such as, but not limited to, any of those listed in Table 3; (hh) a targeted cancer therapy drug; (ii) a tranquilizer, where the tranquilizer includes one or more members such as, but not limited to, any of those listed in Table 3; (jj) a wound healing agent; and (kk) an ion, metal, and/or mineral, wherein the ion, metal, and/or mineral such as, but not limited to, any of those listed in Table 3.

In some embodiments, SBPs for use in therapeutic applications may include or be combined with an engineered tissue. The engineered tissue may include one or more members such as, but not limited to, pancreatic tissue, skeletal muscle tissue, tympanic membrane tissue, bladder tissue, vascular tissue, nervous tissue, neural tissue, corneal tissue, spinal tissue, bone tissue, cartilage tissue, connective tissue, musculoskeletal tissue, cartilaginous tissue, mucosal tissue, vaginal tissue, cardiac tissue, pulmonary tissue, gastrointestinal tissue, dermatologic tissue, retinal tissue, ocular tissue, otic tissue, sinus tissue, pharyngeal tissue, tracheal tissue, liver tissue, renal tissue, splenic tissue, urologic tissue, gynecological tissue, joint tissue, lymphatic tissue, and skin. The SBP may include or be combined with a therapeutic device. The therapeutic device may include one or more members such as, but not limited to, any of those listed in Table 6. The SBP may include or be combined with a gel and/or a hydrogel.

In some embodiments, SBPs for use in agricultural applications may be used in agricultural application that include one or more members such as, but not limited to, (a) farming; (b) plant growth, yield, reproduction, and/or health; (c) preparing and/or applying soil and/or mulch; (d) weed control; (e) pest control; (f) disease control; (g) seed treatment; (h) seed storage; (i) animal growth, yield, reproduction, and/or health; (j) agricultural product preservation and/or treatment; and (k) controlling access to water, air, and/or sunlight. The SBP may include an agricultural composition, where the agricultural composition is formulated for application to one or more members such as, but not limited to, (a) a plant or plant product; (b) a seed; (c) a planting substrate, where the planting substrate includes one or more members such as, but not limited to, soil, mulch, sand, rocks, a sponge, a gel, a matrix, and a mesh; (d) a weed; (e) a pest, a pest habitat, and/or a pest-susceptible surface; (f) a fertilizer; and (g) a device. The agricultural composition may be formulated for application to a plant and/or seed, where the plant and/or seed includes or is derived from one or more members such as, but not limited to, acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, hops, kale, kiwifruit, kohlrabi, larch, lettuce, leek, lemon, lime, locust, pine, maidenhair, maize, mango, maple, marijuana, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. The agricultural composition may be formulated for application to a planting substrate, where the agricultural composition modulates a planting substrate property such as, but not limited to, heat trapping, nutrient content, pH, structure, porosity, active ingredient content, water content, and stability. The application to a planting substrate may include one or more members such as, but not limited to, crop dusting, painting, layering, applying a film, brushing, mixing, spraying, spreading, sprinkling, implanting, and injection. The agricultural composition may be formulated for application to a weed, where the weed may be, but is not limited to, Amaranth, Bermuda grass, Bindweed, Broadleaf plantain, Burdock, Common lambsquarters, Creeping Charlie, Dandelion, Goldenrod, Japanese knotweed, Kudzu, Leafy spurge, Milk thistle, Poison ivy, Ragweed, Sorrel, Striga, St. John's wort, Sumac, Tree of heaven, White clover, Wild carrot, Wood sorrel, and Yellow nutsedge. The agricultural composition may be formulated for application to a pest, pest habitat, and/or a pest-susceptible surface, where the agricultural composition includes at least one pest control agent, where the at least one pest control agent is directed to one or more pests such as, but not limited to, bacteria, fungi, viruses, parasites, insects, arachnids, birds, mammals, and reptiles. The agricultural composition may be formulated for application to a pest habitat, where the pest habitat includes one or more members such as, but not limited to, soil, lawns, gardens, rocks, homes, deserts, tundra, fields, forests, and shrubs. The agricultural composition may be formulated for application to a pest-susceptible surface, where the pest-susceptible surface includes one or more members such as, but not limited to, the ground, water, leaves, branches, stems, bark, moss, fungi, fruits, crops, pine needles, nuts, roots, flowers, and seeds. The agricultural composition may be formulated for application to a fertilizer, where the fertilizer includes one or more members such as, but not limited to, a single-nutrient fertilizer, a binary fertilizer, a multinutrient fertilizer, a nitrogen fertilizer, a phosphate fertilizer, a potassium fertilizer, a compound fertilizer, and an organic fertilizer. The agricultural composition may be formulated for application to a device, where the device includes one or more members such as, but not limited to, delivery devices, agricultural equipment, pest control devices, fencing, plant support structures, watering equipment, netting, storage containers, and bale bags. The agricultural composition may include a coating, where the coating is used for one or more purposes such as, but not limited to, (a) protection of a seed, plant, planting substrate, agricultural product, or device; (b) fertilizing and/or promoting germination of a coated seed or plant; (c) encasing a payload; (d) delivering a payload; (e) modulating nutrient and/or water uptake; (f) stabilizing a payload; and (g) controlling the release of a payload. The agricultural composition may include a coating agent. The coating agent may include one or more compounds such as, but not limited to, polyethylene glycol, methylcellulose, hypromellose, ethylcellulose, gelatin, hydroxypropyl cellulose, titanium dioxide, zein, poly(alkyl)(meth)acrylate, and poly(ethylene-co-vinyl acetate). The agricultural composition may include a coated seed. The agricultural composition may include a payload. The payload may include one or more members such as, but not limited to, any of those listed in Table 7. The agricultural composition may be formulated for delivery of the payload to a target and/or for stabilization of the payload. Delivery of the payload to the target may include delivery by direct contact; by diffusion; by dispersion; by degradation and/or dissolution of the agricultural composition; and/or by controlled release. Delivery of the payload to the target may include delivery by controlled release, where the controlled release includes sustained release of the payload over a delivery period. Delivery of the payload to the target may include delivery by controlled release, where the controlled release includes a desired rate of release of the payload. The agricultural composition may include a photodegradable material. The photodegradable material may be, but not limited to, a film, a microsphere, and a nanosphere.

In some embodiments, SBPs for use in a material science application may include or be combined with a material. The material may include a particle. The particle may include a nanoparticle. The nanoparticle may be, but is not limited to, any of those listed in Table 1. The material may be a coating. The coating may include a coating agent. The coating agent may be, but is not limited to, processed silk, paints, lacquers, adhesives, surfactants, particles, liquids, metals, lipids, oils, proteins, plastics, polymers, insulations, films, membranes, polyethylene glycol, methylcellulose, hypromellose, ethylcellulose, gelatin, hydroxypropyl cellulose, titanium dioxide, zein, poly(alkyl)(meth)acrylate, and/or poly(ethylene-co-vinyl acetate and any of the excipients listed in Table 1. The material may include at least one excipient. The excipient may include one or more members such as, but not limited to, (a) a lipid, lipid nanoparticle, and/or liposome, wherein the lipid, lipid nanoparticle, and/or liposome includes one or more members such as, but not limited to, any of those listed in Table 1; (b) a bulking agent, where the bulking agent includes one or more members such as, but not limited to, any of those listed in Table 1; (c) a sweetener, where the sweetener includes one or more members such as, but not limited to, any of those listed in Table 1; (d) a colorant, where the colorant includes one or more members such as, but not limited to, any of those listed in Table 1; (e) a preservative, where the preservative includes one or more members such as, but not limited to, any of those listed in Table 1; (f) a flowability agent, where the flowability agent includes one or more members such as, but not limited to, any of those listed in Table 1; and (g) a compound or composition selected from one or more members such as, but not limited to, any of those listed in Table 1. The SBP may be combined with a material, where the material includes a plastic, a plastic replacement, a polyolefin, a fabric, an electronic, a device, and/or a food product.

In some embodiments, the present disclosure provides a method of preparing a SBP for use in a therapeutic application, an agricultural application, and/or a material science application, where the SBP includes processed silk, the method including: (a) preparing the processed silk, where the processed silk includes or is derived from one or more articles such as, but not limited to, raw silk, silk fiber, silk fibroin, and a silk fibroin fragment; and (b) preparing the SBP using the processed silk. Preparing the processed silk may include one or more methods selected from the group consisting of: (a) harvesting raw silk from a silk producer, where the silk producer includes a wild type organism or a genetically modified organism; (b) degumming raw silk and/or silk fiber including treating the raw silk and/or silk fiber with degumming solution, wherein the degumming solution includes at least one degumming agent including one or more members such as, but not limited to, water, alcohols, soaps, acids, alkaline solutions, detergents, salts, and enzymes; (c) preparing a processed silk solution, where the processed silk solution includes silk fibroin and a solvent, where the solvent includes one or more members such as, but not limited to, an organic solvent, water, saline, high salt solution, and buffer; (d) purifying and/or concentrating silk fibroin; (e) drying processed silk, where drying is carried out according to a method including one or more members such as, but not limited to, oven drying, lyophilizing, and air drying; and (f) preparing a processed silk format: (i) where the processed silk format includes one or more formats such as, but not limited to, adhesives, capsules, coatings, cocoons, combs, cones, cylinders, discs, emulsions, fibers, films, foams, gels, grafts, hydrogels, implants, mats, membranes, microspheres, nanofibers, nanoparticles, nanospheres, nets, organogels, particles, patches, powders, rods, scaffolds, sheets, solids, solutions, sponges, sprays, spuns, suspensions, tablets, threads, tubes, vapors, and yarns; and (ii) where the processed silk format is prepared by a process including one or more members such as, but not limited to, acidifying, air drying, alkalinizing, annealing, chemical crosslinking, chemical modification, concentration, cross-linking, degumming, dissolving, dry spinning, drying, electrifying, electrospinning, electrospraying, emulsifying, encapsulating, extraction, extrusion, gelation, harvesting, heating, lyophilization, molding, oven drying, pH alteration, precipitation, purification, shearing, sonication, spinning, spray drying, spray freezing, spraying, vapor annealing, vortexing, and water annealing. Preparing the processed silk may include harvesting raw silk from a silk producer, where the silk producer may be, but not limited to, an insect and an arachnid. The silk producer may be an insect, wherein the insect species may be, but not limited to, Bombyx mandarina, Bombyx mori, Bombyx sinesis, Anaphe moloneyi, Anaphe panda, Anaphe reticulate, Anaphe ambrizia, Anaphe carteri, Anaphe venata, Anapha infracta, Antheraea assamensis, Antheraea assama, Antheraea mylitta, Antheraea pernyi, Antheraea yamamai, Antheraea polyphemus, Antheraea oculea, Anisota senatoria, Apis mellifera, Araneus diadematus, Araneus cavaticus, Automeris io, Atticus atlas, Copaxa multifenestrata, Coscinocera hercules, Callosamia promethea, Eupackardia calleta, Eurprosthenops australis, Gonometa postica, Gonometa rufobrunnea, Hyalophora cecropia, Hyalophora euryalus, Hyalophora gloveri, Miranda auretia, Nephila madagascarensis, Nephila clavipes, Pachypasa otus, Pachypasa atus, Philosamia ricini, Pinna squamosa, Rothschildia hesperis, Rothschildia lebeau, Samia cynthia, and Samia ricini. The insect may be Bombyx mori. Preparing the processed silk may include harvesting raw silk from a silk producer, where the silk producer is a genetically modified organism, where the genetically modified organism includes at least one nucleic acid encoding at least one silk protein. The at least one silk protein may include one or more members such as, but not limited to, a silk fibroin heavy chain, a silk fibroin light chain, a silk fibroin fragment, and sericin. The genetically modified organism may be, but is not limited to, an insect, an arachnid, a bacteria, a yeast, a mammalian cell, and a plant cell. Preparing the processed silk may include degumming raw silk and/or silk fiber in degumming solution, where the raw silk and/or silk fiber are heated in the degumming solution. The raw silk and/or silk fiber may be heated in the degumming solution at a temperature of from about 4° C. to about 115° C. The raw silk and/or silk fiber may be heated in degumming solution for a period of from about 10 seconds to about 24 hours. Preparing the processed silk may include preparing a solution of silk fibroin, wherein the solution of silk fibroin includes one or more salts se such as, but not limited to, lithium bromide, lithium thiocyanate, Ajisawa's reagent, a chaotropic agent, and calcium nitrate. Preparing the processed silk may include preparing a solution of silk fibroin, where the solution of silk fibroin may include from about 0.001% (w/v) to about 50% (w/v) silk fibroin. The solution of silk fibroin may be prepared by dissolving silk fibroin in solvent for from about 10 minutes to about 6 hours. The solution of silk fibroin may be prepared by dissolving silk fibroin in solvent at a temperature of from about 4° C. to about 25° C. The solution of silk fibroin may be prepared using one or more chaotropic agents. The one or more chaotropic agents may include one or more members selected from the group consisting of sodium dodecyl sulfate, ethanol, methanol, phenol, 2-propanol, thiourea, urea, n-butanol, zinc chloride, calcium nitrate, lithium perchlorate, lithium acetate, sodium thiocyanate, calcium thiocyanate, magnesium thiocyanate, calcium chloride, magnesium chloride, guanidinium chloride, lithium bromide, lithium thiocyanate, hexafluoroisopropanol, and copper salts. Sucrose, phosphate buffer, tris buffer, trehalose, mannitol, citrate buffer, ascorbate, histidine, and/or a cryoprotective agent may be added to the silk fibroin solution. Preparing the processed silk may include silk fibroin purification and/or concentration by dialysis, centrifugation, air drying, vacuum drying, filtration, and/or Tangential Flow Filtration (TFF). Preparing the processed silk may include preparing a processed silk format by drying a silk fibroin solution. The silk fibroin solution may be dried in an oven at a temperature of from about 30° C. to about 90° C. The silk fibroin solution may be dried for from about 1 hour to about 24 hours. The silk fibroin solution may be dried by one or more methods selected from the group consisting of lyophilization, spray drying, spray freezing, and vacuum drying. The silk fibroin solution may be air dried. The silk fibroin solution may be air dried for from about 1 hour to about 24 hours. Preparing the SBP may include preparing a processed silk format, where the processed silk format includes a rod, where the rod is prepared by extrusion of a silk fibroin composition through an opening. The opening may include a tube. The tube may include a needle. Preparing the SBP may include preparing a processed silk format, where the processed silk format includes hydrogel. The hydrogel may be prepared using a gelling agent. The hydrogel may be prepared using one or more methods selected from the group consisting of ultrasound, sonication, shear force, temperature change, exposure to electrical current, pH modulation, osmolarity modulation, seeding, cross-linking, and chemical modification. Preparing the SBP may include preparing a processed silk format, where the processed silk format includes a rod, where the rod is prepared by a method such as, but not limited to, injection molding, heated or cooled extrusion, extrusion through a coating agent, milling with a therapeutic agent, and combining with a polymer followed by extrusion. The SBP may be prepared by combining the processed silk with one or more articles selected from the group consisting of: (a) an excipient, where the excipient includes one or more members selected from the group consisting of any of those listed in Table 1; (b) a therapeutic agent, wherein the therapeutic agent includes one or more members such as, but not limited to, any of those listed in Table 3; and (c) a device. In some embodiments, the present disclosure provides a SBP prepared by any of the methods described herein.

In some embodiments, the present disclosure provides a method of: (1) treating, preventing, mitigating, alleviating, curing, and/or diagnosing a disease, disorder, and/or condition in a subject; (2) restoring or promoting health, nutrition and/or wellbeing of a subject; and/or (3) supporting or promoting reproduction in a subject, the method including contacting the subject with an SBP described herein. The subject may be selected from the group consisting of any of those listed in Table 2. The SBP may be administered to the subject by a route of administration selected from the group consisting of auricular administration, intraarticular administration, intramuscular administration, intrathecal administration, extracorporeal administration, buccal administration, intrabronchial administration, conjunctival administration, cutaneous administration, dental administration, endocervical administration, endosinusial administration, endotracheal administration, enteral administration, epidural administration, intra-abdominal administration, intrabiliary administration, intrabursal administration, oropharyngeal administration, interstitial administration, intracardiac administration, intracartilaginous administration, intracaudal administration, intracavernous administration, intracerebral administration, intracorporous cavernosum, intracavitary administration, intracorneal administration, intracisternal administration, cranial administration, intracranial administration, intradermal administration, intralesional administration, intratympanic administration, intragingival administration, intraovarian administration, intraocular administration, intradiscal administration, intraductal administration, intraduodenal administration, ophthalmic administration, intradural administration, intraepidermal administration, intraesophageal administration, nasogastric administration, nasal administration, laryngeal administration, intraventricular administration, intragastric administration, intrahepatic administration, intraluminal administration, intravitreal administration, intravesicular administration, intralymphatic administration, intramammary administration, intramedullary administration, intrasinal administration, intrameningeal administration, intranodal administration, intraovarian administration, intrapulmonary administration, intrapericardial administration, intraperitoneal administration, intrapleural administration, intrapericardial administration, intraprostatic administration, intrapulmonary administration, intraluminal administration, intraspinal administration, intrasynovial administration, intratendinous administration, intratesticular administration, subconjunctival administration, intracerebroventricular administration, epicutaneous administration, intravenous administration, retrobulbar administration, periarticular administration, intrathoracic administration, subarachnoid administration, intratubular administration, periodontal administration, transtympanic administration, transtracheal administration, intratumor administration, vaginal administration, urethral administration, intrauterine administration, oral administration, gastroenteral administration, parenteral administration, sublingual administration, ureteral administration, percutaneous administration, peridural administration, transmucosal administration, perineural administration, transdermal administration, rectal administration, soft tissue administration, intraarterial administration, subcutaneous administration, topical administration, extra-amniotic administration, insufflation, enema, eye drops, ear drops, and intravesical infusion. The method may include treating, mitigating, curing, and/or preventing a disease, disorder, and/or condition in a subject, where the disease, disorder, and/or condition is selected from one or more members of the group consisting of any of those listed in Table 5.

In some embodiments, the present disclosure provides a method that includes the use of an SBP described herein for farming; plant growth, yield, reproduction, and/or health; preparing and/or applying soil and/or mulch; weed control; pest control; plant disease control; seed treatment; seed storage; agricultural product preservation and/or treatment; and/or controlling access to water, air, and/or sunlight.

### BRIEF DESCRIPTION OF THE FIGURES

The foregoing and other objects, features and advantages will be apparent from the following description of particular embodiments of the invention, as illustrated in the accompanying drawings. The drawings are not necessarily to scale; emphasis instead being placed upon illustrating the principles of various embodiments of the invention.

FIG. 1A is a scanning electron microscope (SEM) image showing a silk fibroin rod formulated with celecoxib.

FIG. 1B is a scanning electron microscope (SEM) image showing a silk fibroin rod formulated with celecoxib.

FIG. 1C is a scanning electron microscope (SEM) image showing a silk fibroin rod formulated with celecoxib.

FIG. 1D is a scanning electron microscope (SEM) image showing a silk fibroin rod formulated with celecoxib.

FIG. 2A is an image showing a silk fibroin rod formulated with celecoxib, with a diameter of 430 μm.

FIG. 2B is a SEM image showing a silk fibroin rod formulated with celecoxib, with a diameter of 430 μm.

FIG. 2C is a SEM image showing a silk fibroin rod formulated with celecoxib, with a diameter of 430 μm.

FIG. 2D is a SEM image showing a silk fibroin rod formulated with celecoxib, with a diameter of 430 μm.

FIG. 3 is a graph showing TNF-α concentration in human whole blood after administration of various concentrations of lipopolysaccharide (LPS) or silk fibroin.

FIG. 4 is a plot of the cumulative release percentage of an API, celecoxib, over time for a hydrogel and a suspension of celecoxib.

### DETAILED DESCRIPTION

Embodiments of the present disclosure relate to silk-based products (SBPs) and their methods of use. The term “silk” generally refers to a fibrous material formed by insects and some other species that includes tightly bonded protein filaments. Herein, the term “silk” is used in the broadest sense and may embrace any forms, variants, or derivatives of silk discussed

Silk fibers from silkworm moth (Bombyx mori) cocoons include two main components, sericin (usually present in a range of 20-30%) and silk fibroin (usually present in a range of 70-80%). While not wishing to be bound by theory, structurally silk fibroin forms the center of the silk fibers, and sericin acts as the gum coating the fibers. Sericin is a gelatinous protein that holds silk fibers together with many of the characteristic properties of silk (see Qi et al. (2017) Int J Mol Sci 18:237 and Deptuch et al. (2017) Materials 10:1417, the contents of each of which are herein incorporated by reference in their entireties). Silk fibroin is an insoluble fibrous protein consisting of layers of antiparallel beta sheets. Its primary structure mainly consists of recurrent serine, alanine, and glycine repeating units and the isoelectric point of silk fibroin has been determined to be around 4.2. Silk fibroin monomers include a complex of heavy chain (around 350 kDa) and light chain (around 25 kDa) protein components. Typically, the chains are joined by a disulfide bond. With some forms, heavy chain and light chain segments are non-covalently bound to a glycoprotein, p25. Polymers of silk fibroin monomers may form through hydrogen bonding between monomers, typically increasing mechanical strength (see Qi et al. (2017) Int J Mol Sci 18:237). During silk processing, fragments of silk fibroin monomers may be produced, including, but not limited to, fragments of heavy and/or light chains. These fragments may retain the ability to form hydrogen bonds with silk fibroin monomers and fragments thereof. Herein, the term “silk fibroin” is used in its broadest sense and embraces silk fibroin polymers, silk fibroin monomers, silk fibroin heavy and light chains, silk fibroin fragments, and variants, derivatives, or mixtures thereof from any of the wild type, genetically modified, or synthetic sources of silk described herein.

The present disclosure includes methods of preparing processed silk and SBPs, different forms of SBPs, and a variety of applications for utilizing processed silk and SBPs alone or in combination with various compounds, compositions, and devices.

### I. Silk-Based Products

As used herein, the term “silk-based product” or “SBP” refers to any compound, mixture, or other entity that is made up of or that is combined with processed silk. “Processed silk.” as used herein, refers to any forms of silk harvested, obtained, synthesized, formatted, manipulated, or altered through at least one human intervention. SBPs may include a variety of different formats suited for a variety of different applications. Examples of SBP formats include, but are not limited to, fibers, nanofibers, mats, films, foams, membranes, rods, tubes, gels, hydrogels, microspheres, nanospheres, solutions, patches, grafts, adhesives, capsules, cones, cylinders, discs, emulsions, nanoparticles, nets, organogels, particles, scaffolds, sheets, solids, sponges, sprays, spuns, suspensions, tablets, threads, vapors, yarns, and powders. Additional formats are described herein. SBPs may find utility in variety of fields and for a variety of applications. Such utility may be due to the unique physical and chemical properties of silk. These physical and chemical properties include, but are not limited to, biocompatibility, biodegradability, bioresorbability, solubility, crystallinity, porosity, mechanical strength, thermal stability, and transparency. In some embodiments, SBPs may be used for one or more therapeutic applications, agricultural applications, and/or material science applications. Such SBPs may include processed silk, wherein the processed silk is or is derived from one or more of raw silk, silk fibers, silk fibroin, and silk fibroin fragments. Processed silk present is some SBPs may include one or more silk fibroin polymers, silk fibroin monomers, and/or silk fibroin fragments. In some embodiments, silk fibroin fragments include silk fibroin heavy chain fragments and/or silk fibroin light chain fragments. Some silk fibroin present in SBPs include a plurality of silk fibroin fragments. Each of the plurality of silk fibroin fragments may have a molecular weight of from about 1 kDa to about 350 kDa. As a non-limiting example, the silk fibroin fragment may have a molecular weight of 1 kDa, 2 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 105 kDa, 110 kDa, 115 kDa, 120 kDa, 125 kDa, 130 kDa, 135 kDa, 140 kDa, 145 kDa, 150 kDa, 155 kDa, 160 kDa, 165 kDa, 170 kDa, 175 kDa, 180 kDa, 185 kDa, 190 kDa, 195 kDa, 200 kDa, 205 kDa, 210 kDa, 215 kDa, 220 kDa, 225 kDa, 230 kDa, 235 kDa, 240 kDa, 245 kDa, 250 kDa, 255 kDa, 260 kDa, 265 kDa, 270 kDa, 275 kDa, 280 kDa, 285 kDa, 290 kDa, 295 kDa, 300 kDa, 305 kDa, 310 kDa, 315 kDa, 320 kDa, 325 kDa, 330 kDa, 335 kDa, 340 kDa, 345 kDa, or 350 kDa. As a non-limiting example, the silk fibroin fragment may have a molecular weight of 1-5 kDa, 1-10 kDa, 1-15 kDa, 1-25 kDa, 1-50 kDa, 1-75 kDa, 1-100 kDa, 1-150 kDa, 1-200 kDa, 1-250 kDa, 1-300 kDa, 1-350 kDa, 5-10 kDa, 5-15 kDa, 5-25 kDa, 5-50 kDa, 5-75 kDa, 5-100 kDa, 5-150 kDa, 5-200 kDa, 5-250 kDa, 5-300 kDa, 5-350 kDa, 10-15 kDa, 10-25 kDa, 10-50 kDa, 10-75 kDa, 10-100 kDa, 10-150 kDa, 10-200 kDa, 10-250 kDa, 10-300 kDa, 10-350 kDa, 15-25 kDa, 15-50 kDa, 15-75 kDa, 15-100 kDa, 15-150 kDa, 15-200 kDa, 15-250 kDa, 15-300 kDa, 15-350 kDa, 25-50 kDa, 25-75 kDa, 25-100 kDa, 25-150 kDa, 25-200 kDa, 25-250 kDa, 25-300 kDa, 25-350 kDa, 50-75 kDa, 50-100 kDa, 50-150 kDa, 50-200 kDa, 50-250 kDa, 50-300 kDa, 50-350 kDa, 75-100 kDa, 75-150 kDa, 75-200 kDa, 75-250 kDa, 75-300 kDa, 75-350 kDa, 100-150 kDa, 100-200 kDa, 100-250 kDa, 100-300 kDa, 100-350 kDa, 150-200 kDa, 150-250 kDa, 150-300 kDa, 150-350 kDa, 200-250 kDa, 200-300 kDa, 200-350 kDa, 250-300 kDa, 250-350 kDa, and 300-350 kDa

### Sources of Silk

SBPs may include processed silk obtained from one or more of a variety of sources. Processed silk may include raw silk. “Raw silk,” as used herein, refers to silk that has been harvested, purified, isolated, or otherwise collected from silk producers. The term “silk producer,” as used herein, refers to any organism capable of producing silk. Raw silk has been processed in large quantities for thousands of years, primarily from silkworms (Bombyx mori), which use silk to form their cocoon. Raw silk from silkworm cocoons includes silk fibroin and sericin that is secreted onto silk fibroin during cocoon formation. Raw silk may be harvested as a silk fiber. As used herein, the term “silk fiber” refers to any silk that is in the form of a filament or thread. Silk fibers may vary in length and width and may include, but are not limited to, yarns, strings, threads, and nanofibers. In some embodiments, raw silk may be obtained in the form of a yarn.

### Silk Producers

In some embodiments, processed silk includes silk obtained from a silk producer. Silk producers may be organisms found in nature (referred to herein as “wild type organisms”) or they may be genetically modified organisms. There are many species of silk producers in nature capable of producing silk. Silk producers may be insect species, such as silkworms. Some silk producers include arachnid species. In some embodiments, silk producers include species of mollusk. Silk produced by different silk producing species may vary in physical and/or chemical properties. Such properties may include amino acid content, secondary structure (e.g. β-sheet content), mechanical properties (e.g. elasticity), and others. In some embodiments, the present disclosure provides blends of processed silk from multiple silk producers or other sources (e.g., recombinant or synthetic silk). Such blends may have synergistic properties that are absent from processed silk obtained from single sources or from alternative blends. For example, Janani G et al. describe a silk scaffold fabricated by blending Bombyx mori silk fibroin with cell adhesion motif (RGD) rich Antheraea assamensis silk fibroin which displays enhanced liver-specific functions of cultured hepatocytes (Acta Biomater. 2018 February; 67:167-182, the contents of which are herein incorporated by reference in their entirety).

In some embodiments, processed silk may be obtained from the silkworm species Bombyx mori. Other examples of silk producer species include, but are not limited to, Bombyx mandarina, Bombyx sinesis, Anaphe moloneyi, Anaphe panda, Anaphe reticulate, Anaphe ambrizia, Anaphe carteri, Anaphe venata, Anaphe infracta, Antheraea assamensis, Antheraea assama, Antheraea mylitta, Antheraea pernyi, Antheraea yamamai, Antheraea polyphemus, Antheraea oculea, Anisota senatoria, Apis mellifera, Araneus diadematus, Araneus cavaticus, Automeris io, Atticus atlas, Copaxa multifenestrata, Coscinocera hercules, Callosamia promethea, Eupackardia calleta, Eurprosthenops australis, Gonometa postica, Gonometa rufobrunnea, Hyalophora cecropia, Hyalophora euryalus, Hyalophora gloveri, Miranda auretia, Nephila madagascarensis, Nephila clavipes, Pachypasa otus, Pachypasa atus, Philosamia ricini, Pinna squamosa, Rothschildia hesperis, Rothschildia lebeau, Samia cynthia, and Samia ricini.

### Genetically Modified Organisms

In some embodiments, silk producers are genetically modified organisms. As used herein, the term “genetically modified organism” or “GMO” refers to any living entity that includes or is derived from some form of genetic manipulation. The genetic manipulation may include any human intervention that alters the genetic material of an organism. In some embodiments, the genetic manipulation is limited to selecting organisms for reproduction based on genotype or phenotype. In some embodiments, genetic manipulation includes adding, deleting, and/or substituting one or more nucleotides of a wild type DNA sequence. The genetic manipulation may include the use of recombinant DNA technology. Recombinant DNA technology involves the exchange of DNA sections between DNA molecules. Some genetic manipulation involves the transfer of genetic material from another organism to the GMO. GMOs including such transferred genetic material are referred to as “transgenic organisms.” Some genetic materials may be synthetically produced (see e.g., Price et al. (2014) J Control Release 190:304-313; and Deptuch et al. (2017) Materials 10:1417, the contents of each of which are herein incorporated by reference in their entirety). The genetic material may be transferred by way of a vector. The vector may be a plasmid. In some embodiments the vector is a virus. Some genetic manipulations involve the use of inhibitory RNA. In some embodiments, genetic manipulations are carried out using clustered regularly interspaced short palindromic repeats (CRISPR) technology.

GMO silk producers may be species generally known to produce silk (e.g., any of those described above). Some GMO silk producers are species not generally known to produce silk, but that are genetically manipulated to produce silk. Such organisms may be genetically modified to include at least one nucleic acid encoding at least one silk protein (e.g., silk fibroin, silk fibroin heavy chains, silk fibroin light chains, sericin, or fragments or derivates thereof). Some GMO silk producers are genetically manipulated to produce silk with one or more altered silk properties (e.g., strength, stability, texture, etc.). Some genetic manipulations affect characteristics of the GMO that are not directly related to silk production or silk properties (e.g., disease resistance, reproduction, etc.).

In some embodiments, GMO silk producers include genetically modified silkworms (e.g., Bombyx mori). Genetically modified silkworms may include genetic manipulations that result in silkworm production of silk fibroin strands that include degradable linkers. In some embodiments, GMOs are arachnids (e.g., spiders).

In some embodiments, GMO silk producers are cells. Such cells may be grown in culture and may include any type of cell capable of expressing protein. The cells may be prokaryotic or eukaryotic cells. In some embodiments, silk producer cells include bacterial cells, yeast cells, mammalian cells, or plant cells. Cells may be transformed or transduced with nucleic acids encoding one or more silk proteins (e.g., silk fibroin, sericin, or fragments or derivates thereof).

In some embodiments, GMO silk producers may include, but are not limited to, Bombyx mori, soybeans, Arabidopsis, Escherichia coli, Pichia pastoris, potato, tobacco, baby hamster kidney cells, mice, and goats (e.g., see Tokareva et al. (2013) Microb Biotechnol 6(6):651-63 and Deptuch et al. (2017) Materials 10:1417). In some embodiments, silk may be produced in green plants (e.g., see International Publication Number WO2001090389, the contents of which are herein incorporated by reference in their entirety).

### Recombinant Silk

As used herein, the term “recombinant silk” refers to any form of silk produced using recombinant DNA technology. Recombinant silk proteins may include amino acid sequences corresponding to silk proteins produced by wild type organisms; amino acid sequences not found in nature; and/or amino acid sequences found in nature, but not associated with silk. Some recombinant silk includes amino acid sequences with repetitive sequences that contribute to polymer formation and/or silk properties (e.g., see Deptuch et al. (2017) Materials 10:1417). Nucleic acid segments encoding repetitive sequences may be incorporated into plasmids after self-ligation into multimers (e.g., see Price et al. (2014) J Control Release 190:304-313). In some embodiments, recombinant silk may be encoded by expression plasmids.

In some embodiments, recombinant silk may be expressed as a monomer. The monomers may be combined with other monomers or other silk proteins to obtain multimers (e.g., see Deptuch et al. (2017) Materials 10:1417). Some monomers may be combined according to methods known in the art. Such methods may include, but are not limited to, ligation, step-by-step ligation, recursive directional ligation, native chemical ligation, and concatemerization.

In some embodiments, recombinant silk may be expressed using the “PiggyBac” vector. The PiggyBac vector includes a spider transposon that is compatible with expression in silkworms.

In some embodiments, recombinant silk may be produced in a silk producing species. Examples of silk producing species include, but are not limited to, Bombyx mori, Bombyx mandarina, Bombyx sinesis, Anaphe moloneyi, Anaphe panda, Anaphe reticulate, Anaphe ambrizia, Anaphe carteri, Anaphe venata, Anapha infracta, Antheraea assamensis, Antheraea paphis, Antheraea assama, Antheraea mylitta, Antheraea pernyi, Antheraea yamamai, Antheraea polyphemus, Antheraea oculea, Anisota senatoria, Apis mellifera, Araneus diadematus, Araneus cavaticus, Automeris io, Atticus atlas, Coscinocera hercules, Callosamia promethea, Copaxa multifenestrata, Eupackardia calleta, Eurprosthenops australis, Gonometa postica, Gonometa rufobrunnea, Hyalophora cecropia, Hyalophora euryalus, Hyalophora gloveri, Miranda auretia, Nephila madagascarensis, Nephila clavipes, Pachypasa otus, Pachypasa atus, Philosamia ricini, Pinna squamosa, Rothschildia hesperis, Rothschildia lebeau, Samia cynthia, and Samia ricini.

### Synthetic Silk

In some embodiments, SBPs include synthetic silk. As used herein, the term “synthetic silk” refers to silk prepared without the aid of a silk producer. Synthetic silk may be prepared using standard methods of peptide synthesis. Such methods typically include the formation of amino acid polymers through successive rounds of polymerization. Amino acids used may be obtained through commercial sources and may include natural or non-natural amino acids. In some embodiments, synthetic silk polypeptides are prepared using solid-phase synthesis methods. The polypeptides may be linked to resin during synthesis. Polypeptide synthesis may be automated.

Synthetic silk may include polypeptides that are identical to wild type silk proteins (e.g., silk fibroin heavy chain, silk fibroin light chain, or sericin) or fragments thereof. In some embodiments, synthetic silk includes polypeptides that are variants of silk proteins or silk protein fragments. Some synthetic silk includes polypeptides with repeating units that correspond with or are variations of those found in silk fibroin heavy chain proteins.

### Silk Properties

In some embodiments, processed silk may be selected based on or prepared to include features affecting one or more properties of the processed silk. Such properties may include, but are not limited to, stability, complex stability, composition stability, payload retention or release, payload release rate, wettability, mechanical strength, tensile strength, elongation capabilities, elasticity, compressive strength, stiffness, shear strength, toughness, thickness, density, viscosity, torsional stability, temperature stability, moisture stability, strength, flexibility, solubility, crystallinity, and porosity. Features affecting one or more processed silk properties may include silk secondary structure. Secondary structure refers to three-dimensional arrangements of polypeptide chains based on local interactions between neighboring residues. Common secondary structures include β-pleated sheets and α-helices. Silk secondary structure may enhance or attenuate solubility. In some embodiments, β-sheet secondary structure content may enhance processed silk crystallinity. “Crystallinity” refers to the degree of structure and arrangement between atoms or molecules in a compound, with increased structure yielding greater crystallinity. β-sheet structures may be antiparallel β-sheets. In some embodiments, processed silk includes polypeptides with random coil secondary structure. Some processed silk includes polypeptides with coiled coil secondary structure. In some embodiments, processed silk includes a combination of two or more forms of secondary structure. In some embodiments, processed silk may include polypeptides with multiple repeats. As used herein when referring to polypeptides, the term “multiple repeat” refers to an amino acid sequence that is duplicated two or more times in succession within a polypeptide. Silk fibroin heavy chains include multiple repeats that enable static interactions between parallel silk fibroin heavy chains. Multiple repeats may include repeats of the sequences GAGAGS (SEQ ID NO: 1) and/or GA. In some embodiments, the A of GA dipeptides may be replaced with S or Y. In some embodiments, multiple repeats may include any of those presented in Qi et al. (2017) Int J Mol Sci 18:237, the contents of which are herein incorporated by reference in their entirety. Multiple repeats may enable formation of stable, crystalline regions of antiparallel β-sheets.

Processed silk may include silk fibroin forms described by Qi et al. (2017) Int J Mol Sci 18:237 and Cao et al. (2009) Int J Mol Sci 10:1514-1524, the contents of each of which are herein incorporated by reference in their entirety. These silk fibroin forms are referred to as silk I, silk II, and silk III. Silk I and silk II forms are commonly found in nature. Silk I predominantly includes random coil secondary structures. Silk II predominantly includes β-sheet secondary structure. Silk III predominantly includes an unstable structure.

Processed silk may be treated to modulate α-sheet content and/or crystallinity. In some embodiments these treatments are used to reduce the solubility of the silk fibroin or silk fibroin composition. Treatments may include, but are not limited to, alteration of the pH, sonication of the silk fibroin, incorporation of an excipient, increasing or decreasing the temperature, treatment with acid, treatment with formic acid, treatment with glycerol, treatment with an alcohol, treatment with methanol, treatment with ethanol, treatment with isopropanol, and/or treatment with a mixture of alcohol and water. In some embodiments, treatments result in transition between forms of silk I, II, or III. Such methods may include any of those described in Cao et al. (2009) Int J Mol Sci 10:1514-1524).

### Porosity

In some embodiments, processed silk may include variations in porosity. As used herein, the term “porosity” refers to the frequency with which holes, pockets, channels, or other spaces occur in a material, in some cases influencing the movement of elements to and/or from the material. Processed silk porosity may influence one or more other silk properties or properties of an SBP that includes the processed silk. These properties may include, but are not limited to, stability, payload retention or release, payload release rate, wettability, mechanical strength, tensile strength, elongation capabilities, density, thickness, elasticity, compressive strength, stiffness, shear strength, toughness, torsional stability, temperature stability, and moisture stability. In some embodiments, processed silk porosity may control the diffusion or transport of agents from, within, or into the processed silk or SBP. Such agents may include, but are not limited to, therapeutics, biologics, chemicals, small molecules, oxidants, antioxidants, macromolecules, microspheres, nanospheres, cells, or any payloads described herein.

Processed silk porosity may be modulated during one or more processing steps or during fabrication of a SBP (e.g., see International Publication No. WO2014125505 and U.S. Pat. No. 8,361,617, the contents of each of which are herein incorporated by reference in their entirety). In some embodiments, processed silk porosity may be modulated by one or more of sonication, centrifugation, modulating silk fibroin concentration, modulating salt concentration, modulating pH, modulating secondary structural formats, applying shear stress, modulating excipient concentration, chemical modification, crosslinking, or combining with cells, bacteria, and/or viral particles.

### Strength and Stability

Processed silk strength and stability are important factors for many applications. In some embodiments, processed silk may be selected based on or prepared to maximize mechanical strength, tensile strength, elongation capabilities, elasticity, flexibility, compressive strength, stiffness, shear strength, toughness, torsional stability, biological stability, resistance to degradation, and/or moisture stability. In some embodiments, processed silk has a non-acidic microenvironment. In some embodiments, the non-acidic microenvironment enhances the stability of processed silk and or SBPs. In some embodiments, the non-acidic microenvironment enhances the stability of therapeutic agents formulated with processed silk and/or SBP. In some embodiments, the tensile strength of processed silk is stronger than steel. In some embodiments, the tensile strength of an SBP is stronger than steel.

### Biocompatibility

In some embodiments, processed silk may be selected based on or prepared to maximize biocompatibility. As used herein, the term “biocompatibility” refers to the degree with which a substance avoids provoking a negative biological response in an organism exposed to the substance. The negative biological response may include an inflammatory response, local sensitization, hemorrhage, and/or other complications known to those skilled in the art. In some embodiments, administration of processed silk or an SBP does not induce an inflammatory response, local sensitization, hemorrhage, and/or other complications known to those skilled in the art. In some embodiments, contact with processed silk or an SBP does not induce an inflammatory response, local sensitization, hemorrhage, and/or other complications known to those skilled in the art. In some embodiments, processed silk biocompatibility is enhanced through preparations that produce only non-toxic byproducts during degradation. In some embodiments, exposure to an SBP generates a tolerable biological response, within an acceptable threshold known to those skilled in the art. In some embodiments, processed silk is biocompatible in humans and human whole blood. In some embodiments, processed silk is biocompatible in animals. In some embodiments, processed silk produces no adverse reactions, no acute inflammation, and no immunogenicity in vivo. In some embodiments, the processed silk or SBP is safe to use in vivo. In some embodiments, processed silk or SBPs are biocompatible and/or tolerable in vitro. In some embodiments, processed silk or SBPs are biocompatible and/or tolerable in vivo. In some embodiments, no inflammatory response, local sensitization, hemorrhage, and/or other complications occur after up to 1 day, up to 3 days, up to 1 week, up to 1 month, up to 3 months, up to 4 months, up to 6 months, up to 7 months, or up to 1 year of contact with processed silk or an SBP.

### Biodegradability

In some embodiments, processed silk may be selected based on or prepared to maximize biodegradability. As used herein, the term “biodegradability” refers to the degree with which a substance avoids provoking a negative response to an environment exposed to the substance as it deteriorates. The negative environmental response may include a response to toxic byproducts generated as a substance deteriorates. In some embodiments, processed silk biodegradability is enhanced through preparations that produce only non-toxic byproducts during degradation. In some embodiments, processed silk biodegradability is enhanced through preparations that produce only inert amino acid byproducts. In some embodiments, the SBP and/or SBP by products are considered naturally derived and environmentally and/or eco-friendly.

### Surfactant Properties

In some embodiments, processed silk and/or SBPs may act as a surfactant. As used herein, the term “surfactant” refers to a substance that reduces the surface tension between two materials. In some embodiments, an SBP has a surface tension similar to that of water. In some embodiments, an SBP has a surface tension similar to that of human tears. In some embodiments, the surface tension of an SBP may be controlled by the concentration of processed silk.

### Anti-Evaporative Properties

In some embodiments, processed silk may be selected based on or prepared to reduce the evaporation of a solution. In some embodiments, processed silk may reduce the evaporation of a solution. In some embodiments, an SBP may demonstrate anti-evaporative properties by creating a water barrier. In some embodiments, processed silk may extend the lifetime or residence time of an SBP product due to its ability to prevent evaporation. In some embodiments, processed silk may increase the amount of time required for a solution to evaporate. In some embodiments, processed silk may be selected based on or prepared to reduce the evaporation of a solution. In some embodiments, processed silk may reduce the evaporation of a solution. In some embodiments, processed silk may extend the lifetime or residence time of an SBP product due to its ability to prevent evaporation. In some embodiments, processed silk may increase the amount of time required for a solution to evaporate.

### Antimicrobial Properties

In some embodiments, processed silk may be based on or prepared to maximize antimicrobial properties. As used herein, the term “antimicrobial” properties refer to the ability of processed silk or SBPs to inhibit, deter the growth of microorganisms and/or kill the microorganisms. Microorganisms may include bacteria, fungi, protozoans, and viruses. In some embodiments, the antimicrobial properties may include but are not limited to antibacterial, antifungal, antiseptic, and/or disinfectant properties. In some embodiments, antimicrobial properties of silk may be modulated during one or more processing steps or during fabrication of a SBP. In some embodiments, antimicrobial properties may be modulated by the varying the source of silk utilized for the preparation of SBPs (Mirghani, M et al. 2012, Investigation of the spider web of antibacterial activity, (MICOTriBE) 2012; the contents of which are incorporated by reference in their entirety). In some embodiments, processed silk and SBPs described herein may possess antimicrobial properties against gram positive bacteria. In some embodiments, processed silk and SBPs described herein may possess antimicrobial properties against gram negative bacteria.

### Anti-Inflammatory Properties

In some embodiments, processed silk or SBPs may have or be prepared to maximize anti-inflammatory properties. It has been reported that silk fibroin peptide derived from silkworm Bombyx mori exhibited anti-inflammatory activity in a mice model of inflammation (Kim et al., (2011) BMB Rep 44(12):787-92; the contents of which are incorporated by reference in their entirety). In some embodiments, processed silk or SBPs may be administered to a subject alone or in combination with other therapeutic agents to elicit anti-inflammatory effects. It is contemplated that processed silk or SBPs alone or combination with other therapeutic agents may be used to treat various inflammatory diseases. For example, processed silk or SBPs may reduce signs and symptoms of inflammation, such as but not limited to, swelling, redness, tenderness, rashes, fever, and pain.

### Processed Silk and Related Methods

Various processing methods may be used to obtain specific forms or formats of processed silk. Such processing methods may include, but are not limited to, acidifying, air drying, alkalinizing, annealing, autoclaving, chemical crosslinking, chemical modification, concentration, cross-linking, degumming, dissolving, dry spinning, drying, electrifying, electrospinning, electrospraying, emulsifying, encapsulating, extraction, extrusion, gelation, harvesting, heating, lyophilization, molding, oven drying, pH alteration, precipitation, purification, shearing, sonication, spinning, spray drying, spray freezing, spraying, vapor annealing, vortexing, and water annealing. The processing steps may be used to prepare final SBPs or they may be used to generate processed silk preparations. As used herein, the term “processed silk preparation” is generally used to refer to processed silk or compositions that include processed silk that are prepared for or obtained during or after one or more processing steps. Processed silk preparations may be SBPs, may be components of SBPs, or may be used as a starting or intermediate composition in the preparation of SBPs. Processed silk preparations may include other components related to processing (e.g., solvents, solutes, impurities, catalysts, enzymes, intermediates, etc.). Processed silk preparations that include silk fibroin may be referred to as silk fibroin preparations. In some embodiments, processed silk manufacturing is simple, scalable, and/or cost effective.

In some embodiments, processed silk may be prepared as, provided as, or included in a yarn, thread, string, a nanofiber, a particle, a nanoparticle, a microsphere, a nanosphere, a powder, a solution, a gel, a hydrogel, an organogel, a mat, a film, a foam, a membrane, a rod, a tube, a patch, a sponge, a scaffold, a capsule, an excipient, an implant, a solid, a coating, and/or a graft.

In some embodiments, the formulations are prepared to be sterile. As used herein, the term “sterile” refers to something that is aseptic. In some embodiments, SBPs are prepared from sterile materials. In some embodiments, SBPs are prepared and then sterilized. In some embodiments, processed silk is degummed and then sterilized. In some embodiments, processed silk is sterilized and then degummed. Processed silk and/or SBPs may be sterilized via gamma radiation, autoclave (e.g., autoclave sterilization), filtration, electron beam, and any other method known to those skilled in the art.

In some embodiments, processed silk may be stored frozen or dried to a stable soluble form. Processed silk may be frozen with cryoprotectants. Cryoprotectants may include, but are not limited to, phosphate buffer, sucrose, histidine, and any other cryoprotectant known to one of skill in the art. In some embodiments, SBPs may be stored frozen or dried to a stable soluble form. In some embodiments, the SBPs may be solutions.

In some embodiments, preparation of processed silk and/or SBP formulations may be scaled up for manufacturing at a large scale. In some embodiments, production of processed silk and/or SBP formulations may be accomplished with automated machinery.

Any of the methods known in the art and/or described herein may be used to extract silk fibroin. The yield of silk fibroin from extraction may be, but is not limited to, 1%, 2% 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or greater than 99%.

### Harvesting Silk

In some embodiments, processed silk is harvested from silk producer cocoons. Cocoons may be prepared by cultivating silkworm moths and allowing them to pupate. Once fully formed, cocoons may be treated to soften sericin and allow for unwinding of the cocoon to form raw silk fiber. The treatment may include treatment with hot air, steam, and/or boiling water. Raw silk fibers may be produced by unwinding multiple cocoons simultaneously. The resulting raw silk fibers include both silk fibroin and sericin. Subsequent processing may be carried out to remove sericin from the raw silk fibers or from later forms of processed silk or SBPs. In some embodiments, raw silk may be harvested directly from the silk glands of silk producers. Raw silk may be harvested from wild type or GMO silk producers.

### Extraction of Sericin/Degumming

In some embodiments, sericin may be removed from processed silk, a process referred to herein as “degumming.” The processed silk may include raw silk, which includes sericin secreted during cocoon formation. Methods of degumming may include heating (e.g., boiling) in a degumming solution. As used herein, the term “degumming solution” refers to a composition used for sericin removal that includes at least one degumming agent. As used herein, a “degumming agent” refers to any substance that may be used for sericin removal. Heating in degumming solution may reduce or eliminate sericin from processed silk. In some embodiments, heating in degumming solution includes boiling. Heating in degumming solution may be followed by rinsing to enhance removal of sericin that remains after heating. In some embodiments, raw silk is degummed before further processing or utilization in SBPs. In other embodiments, raw silk is further processed or otherwise incorporated into a SBP prior to degumming. Such methods may include any of those presented in European Patent No. EP2904134 or United States Publication No. US2017031287, the contents of each of which are herein incorporated by reference in their entirety.

Degumming agents and/or degumming solution may include, but are not limited to water, alcohols, soaps, acids, alkaline solutions, and enzyme solutions. In some embodiments, degumming solutions may include salt-containing alkaline solutions. Such solutions may include sodium carbonate. Sodium carbonate concentration may be from about 0.01 M to about 0.3 M. In some embodiments, sodium carbonate concentration may be from about 0.01 M to about 0.05 M, about 0.05 M to about 0.1 M, from about 0.1 M to about 0.2 M, or from about 0.2 M to about 0.3 M. In some embodiments, sodium carbonate concentration may be 0.02 M. In some embodiments, degumming solutions may include from about 0.01% to about 1% (w/v) sodium carbonate. In some embodiments, degumming solutions may include from about 0.01% to about 10% (w/v) sodium carbonate. In some embodiments, degumming solutions may include from about 0.01% (w/v) to about 1% (w/v), from about 1% (w/v) to about 2% (w/v), from about 2% (w/v) to about 3% (w/v), from about 3% (w/v) to about 4% (w/v), from about 4% (w/v) to about 5% (w/v), or from about 5% (w/v) to about 10% (w/v) sodium carbonate. In some embodiments, degumming solutions may include sodium dodecyl sulfate (SDS). Such degumming solutions may include any those described in Zhang et al. (2012) J Translational Med 10:117, the contents of which are herein incorporated by reference in their entirety. In some embodiments, degumming solutions include boric acid. Such solutions may include any of those taught in European Patent No. EP2904134, the contents of which are herein incorporated by reference in their entirety. In some embodiments, the degumming solution may have a pH of from about 0 to about 5, from about 2 to about 7, from about 4 to about 9, from about 5 to about 11, from about 6 to about 12, from about 6.5 to about 8.5, from about 7 to about 10, from about 8 to about 12, and from about 10 to about 14. In some embodiments, processed silk may be present in degumming solutions at concentrations of from about 0.1% to about 2%, from about 0.5% to about 3%, from about 1% to about 4%, or from about 2% to about 5% (w/v). In some embodiments, processed silk is present in degumming solutions at concentrations of greater than 5% (w/v).

Degumming may be carried out by boiling in degumming solutions at or near (e.g., within about 5% of) atmospheric boiling temperatures. Some boiling temperatures may be from about 60° C. to about 115° C. In some embodiments, boiling is carried out at 100° C. In some embodiments, boiling is carried out at about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C. about 108° C., about 109° C., or about 110° C.

In some embodiments, degumming includes heating in degumming solution for a period of from about 10 seconds to about 45 seconds, from about 30 seconds to about 90 seconds, from about 1 min to about 5 min, from about 2 min to about 10 min, from about 5 min to about 15 min, from about 10 min to about 25 min, from about 20 min to about 35 min, from about 30 min to about 50 min, from about 45 min to about 75 min, from about 60 min to about 95 min, from about 90 min to about 125 min, from about 120 min to about 175 min, from about 150 min to about 200 min, from about 180 min to about 250 min, from about 210 min to about 350 min, from about 240 min to about 400 min, from about 270 min to about 450 min, from about 300 min to about 500 min, from about 330 min to about 550 min, from about 360 min to about 600 min, from about 390 min to about 700 min, from about 420 min to about 800 min, from about 450 min to about 900 min, from about 480 min to about 1000 min, from about 510 min to about 1100 min, from about 540 min to about 1200 min, from about 570 min to about 1300 min, from about 600 min to about 1400 min, from about 630 min to about 1500 min, from about 660 min to about 1600 min, from about 690 min to about 1700 min, from about 720 min to about 1800 min, from about 1440 min to about 1900 min, from about 1480 min to about 2000 min, or longer than 2000 min.

In some embodiments, processed silk preparations may be characterized by the number of minutes boiling was carried out for preparation, a value referred to herein as “minute boil” or “mb.” The minute boil value of a preparation may be associated with known or presumed characteristics of similar preparations with the same minute boil value. Such characteristics may include concentration and/or molecular weight of preparation compounds, proteins, or protein fragments altered during boiling. In some embodiments, processed silk preparations (e.g., silk fibroin preparations) have an mb value of from about 1 mb to about 5 mb, from about 2 mb to about 10 mb, from about 5 mb to about 15 mb, from about 10 mb to about 25 mb, from about 20 mb to about 35 mb, from about 30 mb to about 50 mb, from about 45 mb to about 75 mb, from about 60 mb to about 95 mb, from about 90 mb to about 125 mb, from about 120 mb to about 175 mb, from about 150 mb to about 200 mb, from about 180 mb to about 250 mb, from about 210 mb to about 350 mb, from about 240 mb to about 400 mb, from about 270 mb to about 450 mb, from about 300 mb to about 480 mb, or greater than 480 mb.

In some embodiments, degumming is carried out by treatment with high temperatures and/or pressures. Such methods may include any of those presented in International Publication No. WO2017200659, the contents of which are herein incorporated by reference in their entirety.

### Processed Silk Preparation Characterization

Preparations of processed silk may include mixtures of silk fibroin polymers, silk fibroin monomers, silk fibroin heavy chains, silk fibroin light chains, sericin, and/or fragments of any of the foregoing. Where the exact contents and ratios of components in such processed silk preparations are unknown, the preparations may be characterized by one or more properties of the preparation or by conditions or methods used to obtain the preparations. As a non-limiting example, the sericin content in the SBP formulation may be 0%, 0.5%, 1%, 1.5%, 2%, 2.5%, 3%, 3.5%, 4% or greater than 4%, or in the range of 0-1%, or 0-2%.

### Solubility and Concentration

Processed silk preparations may include solutions that include processed silk (also referred to herein as “processed silk solutions”). Processed silk solutions may be characterized by processed silk concentration. For example, processed silk may be dissolved in a solvent after degumming to generate a processed silk solution of silk fibroin for subsequent use. Solvent used to dissolve processed silk may be a buffer. In some embodiments, solvent used is an organic solvent. Organic solvents may include, but are not limited to hexafluoroisopropanol (HFIP), methanol, isopropanol, ethanol, or combinations thereof. In some embodiments, solvents include a mixture of an organic solvent and water or an aqueous solution. Solvents may include water or aqueous solutions. Aqueous solutions may include aqueous salt solutions that include one or more salts. Such salts may include but are not limited to lithium bromide (LiBr), lithium thiocyanate, Ajisawa's reagent, a chaotropic agent, calcium nitrate, or other salts capable of solubilizing silk, including any of those disclosed in U.S. Pat. No. 9,623,147 (the content of which is herein incorporated by reference in its entirety). In some embodiments, solvents used in processed silk solutions may include Ajisawa's reagent, as described in Zheng et al. (2016) Journal of Biomaterials Applications 31:450-463, the content of which is herein incorporated by reference in its entirety. Ajisawa's reagent comprises a mixture of calcium chloride, ethanol, and water in a molar ratio of 1:2:8 respectively. In some embodiments, solvents used in processed silk solutions include high salt solutions. In some embodiments, the solution comprises 5 to 13 M LiBr. The concentration of LiBr may be 9.3 M.

In some embodiments, processed silk is present in processed silk solutions at a concentration of from about 0.01% (w/v) to about 1% (w/v), from about 0.05% (w/v) to about 2% (w/v), from about 1% (w/v) to about 5% (w/v), from about 2% (w/v) to about 10% (w/v), from about 4% (w/v) to about 16% (w/v), from about 5% (w/v) to about 20% (w/v), from about 8% (w/v) to about 24% (w/v), from about 10% (w/v) to about 30% (w/v), from about 12% (w/v) to about 32% (w/v), from about 14% (w/v) to about 34% (w/v), from about 16% (w/v) to about 36% (w/v), from about 18% (w/v) to about 38% (w/v), from about 20% (w/v) to about 40% (w/v), from about 22% (w/v) to about 42% (w/v), from about 24% (w/v) to about 44% (w/v), from about 26% (w/v) to about 46% (w/v), from about 28% (w/v) to about 48% (w/v), from about 30% (w/v) to about 50% (w/v), from about 35% (w/v) to about 55% (w/v), from about 40% (w/v) to about 60% (w/v), from about 45% (w/v) to about 65% (w/v), from about 50% (w/v) to about 70% (w/v), from about 55% (w/v) to about 75% (w/v), from about 60% (w/v) to about 80% (w/v), from about 65% (w/v) to about 85% (w/v), from about 70% (w/v) to about 90% (w/v), from about 75% (w/v) to about 95% (w/v), from about 80% (w/v) to about 96% (w/v), from about 85% (w/v) to about 97% (w/v), from about 90% (w/v) to about 98% (w/v), from about 95% (w/v) to about 99% (w/v), from about 96% (w/v) to about 99.2% (w/v), from about 97% (w/v) to about 99.5% (w/v), from about 98% (w/v) to about 99.8% (w/v), from about 99% (w/v) to about 99.9% (w/v), or greater than 99.9% (w/v). In some embodiments, the processed silk is silk fibroin.

Processed silk solutions may be characterized by the length of time and/or temperature needed for processed silk to dissolve. The length of time used to dissolve processed silk in solvent is referred to herein as “dissolution time.” Dissolution times for dissolution of processed silk in various solvents may be from about 1 min to about 5 min, from about 2 min to about 10 min, from about 5 min to about 15 min, from about 10 min to about 25 min, from about 20 min to about 35 min, from about 30 min to about 50 min, from about 45 min to about 75 min, from about 60 min to about 95 min, from about 90 min to about 125 min, from about 120 min to about 175 min, from about 150 min to about 200 min. from about 180 min to about 250 min, from about 210 min to about 350 min, from about 240 min to about 360 min, from about 270 min to about 420 min, from about 300 min to about 480 min, or longer than 480 minutes.

The temperature used to dissolve processed silk in solvent is referred to herein as “dissolution temperature.” Dissolution temperatures used for dissolution of processed silk in solvent may include room temperature. In some embodiments, dissolution temperature may be from about 0° C. to about 10° C., from about 4° C. to about 25° C., from about 20° C. to about 35° C., from about 30° C. to about 45° C., from about 40° C. to about 55° C., from about 50° C. to about 65° C., from about 60° C. to about 75° C. from about 70° C. to about 85° C., from about 80° C. to about 95° C., from about 90° C. to about 105° C., from about 100° C. to about 115° C., from about 110° C. to about 125° C., from about 120° C. to about 135° C., from about 130° C. to about 145° C., from about 140° C. to about 155° C., from about 150° C. to about 165° C., from about 160° C. to about 175° C., from about 170° C. to about 185° C., from about 180° C. to about 200° C., or greater than 200° C. In some embodiments, the processed silk is silk fibroin. Dissolution of some processed silk solutions may use a dissolution temperature of 60° C. Dissolution of some processed silk solutions may use a dissolution temperature of 80° C., as described in Zheng et al. (2016) Journal of Biomaterials Applications 31:450-463. In some embodiments, dissolution includes boiling. In some embodiments, dissolution may be carried out by autoclaving. In some embodiments, silk fibroin solutions may be prepared according to any of the methods described in International Publication Numbers WO2016029034, WO2017200659, and WO2018031973, U.S. Pat. Nos. 9,394,355, and 9,907,836, US Publication Number US20180193429 or Abdel-Naby (2017) PLoS One 12(11): e0188154), the contents of each of which are herein incorporated by reference in their entirety. For example, silk fibroin may be autoclaved while it is combined with lithium bromide (LiBr) in an aqueous solution. The aqueous solution may contain LiBr at a concentration of about 8M to about 10M. Silk fibroin solution may be heated to a temperature in the range of about 105 to about 125° C. under a pressure of about 10 PSI to about 20 PSI. Silk fibroin solution may be heated for any desired duration of time, e.g., for about 10 minutes, about 20 minutes, about 30 minutes, about 45 minutes, about 1 hour, or longer than 1 hour.

In some embodiments, one or more of sucrose, phosphate buffer, tris buffer, trehalose, mannitol, citrate buffer, ascorbate, histidine, and/or a cryoprotective agent is added to processed silk solutions.

### Chaotropic Agents

In some embodiments, processed silk may be dissolved with the aid of a chaotropic agent. As used herein, a “chaotropic agent” refers to a substance that disrupts hydrogen bonding networks in aqueous solutions to facilitate dissolution of a solute. Chaotropic agents typically modify the impact of hydrophobicity on dissolution. Chaotropic agents may be organic compounds. Such compounds may include, but are not limited to, sodium dodecyl sulfate, ethanol, methanol, phenol, 2-propanol, thiourea, urea, n-butanol, and any other chemicals capable of solubilizing silk. In some embodiments, the chaotropic agent is a salt, including, but not limited to, zinc chloride, calcium nitrate, lithium perchlorate, lithium acetate, sodium thiocyanate, calcium thiocyanate, magnesium thiocyanate, calcium chloride, magnesium chloride, guanidinium chloride, lithium bromide, lithium thiocyanate, copper salts, and other salts capable of solubilizing silk. Such salts typically create high ionic strength in the aqueous solutions which destabilizes the beta-sheet interactions in silk fibroin. In some embodiments, a combination of chaotropic agents is used to facilitate the dissolution of silk fibroin. In some embodiments, a chaotropic agent is used to dissolve raw silk during processing.

### Molecular Weight

In some embodiments, processed silk preparations are characterized by the molecular weight of proteins present in the preparations. Different molecular weights may be present as a result of different levels of silk fibroin dissociation and/or fragmentation during degumming or other processing. When referring to silk fibroin molecular weight herein, it should be understood that the molecular weight may be associated with silk fibroin polymers, silk fibroin monomers, silk fibroin heavy and/or light chains, silk fibroin fragments, or variants, derivates, or mixtures thereof. Accordingly, silk fibroin molecular weight values may vary depending on the nature of the silk fibroin or silk fibroin preparation. In some embodiments, processed silk preparations are characterized by average molecular weight of silk fibroin fragments present in the preparation; by a range of silk fibroin fragment molecular weights; by a threshold of silk fibroin fragment molecular weights; or by combinations of averages, ranges, and thresholds.

In some embodiments, processed silk preparation may include silk fibroin with a molecular weight of, average molecular weight of, upper molecular weight threshold of, lower molecular weight threshold of, or range of molecular weights with an upper or lower range value of from about 1 kDa to about 4 kDa, from about 2 kDa to about 5 kDa, from about 3.5 kDa to about 10 kDa, from about 5 kDa to about 20 kDa, from about 7.5 kDa to about 32.5 kDa, from about 7.5 kDa to about 50 kDa, from about 7.5 kDa to about 100 kDa, from about 7.5 kDa to about 150 kDa, from about 7.5 kDa to about 200 kDa, from about 7.5 kDa to about 250 kDa, from about 10 kDa to about 35 kDa, from about 15 kDa to about 40 kDa, from about 20 kDa to about 45 kDa, from about 25 kDa to about 50 kDa, from about 30 kDa to about 55 kDa, from about 35 kDa to about 60 kDa, from about 40 kDa to about 65 kDa, from about 45 kDa to about 70 kDa, from about 50 kDa to about 75 kDa, from about 55 kDa to about 80 kDa, from about 60 kDa to about 85 kDa, from about 65 kDa to about 90 kDa, from about 70 kDa to about 95 kDa, from about 75 kDa to about 100 kDa, from about 80 kDa to about 105 kDa, from about 85 kDa to about 110 kDa, from about 90 kDa to about 115 kDa, from about 95 kDa to about 120 kDa, from about 100 kDa to about 125 kDa, from about 105 kDa to about 130 kDa, from about 110 kDa to about 135 kDa, from about 115 kDa to about 140 kDa, from about 120 kDa to about 145 kDa, from about 125 kDa to about 150 kDa, from about 130 kDa to about 155 kDa, from about 135 kDa to about 160 kDa, from about 140 kDa to about 165 kDa, from about 145 kDa to about 170 kDa, from about 150 kDa to about 175 kDa, from about 160 kDa to about 200 kDa, from about 170 kDa to about 210 kDa, from about 180 kDa to about 220 kDa, from about 190 kDa to about 230 kDa, from about 200 kDa to about 240 kDa, from about 210 kDa to about 250 kDa, from about 220 kDa to about 260 kDa, from about 230 kDa to about 270 kDa, from about 240 kDa to about 280 kDa, from about 250 kDa to about 290 kDa, from about 260 kDa to about 300 kDa, from about 270 kDa to about 310 kDa, from about 280 kDa to about 320 kDa, from about 290 kDa to about 330 kDa, from about 300 kDa to about 340 kDa, from about 310 kDa to about 350 kDa, from about 320 kDa to about 360 kDa, from about 330 kDa to about 370 kDa, from about 340 kDa to about 380 kDa, from about 350 kDa to about 390 kDa, from about 360 kDa to about 400 kDa, from about 370 kDa to about 410 kDa, from about 380 kDa to about 420 kDa, from about 390 kDa to about 430 kDa, from about 400 kDa to about 440 kDa, from about 410 kDa to about 450 kDa, from about 420 kDa to about 460 kDa, from about 430 kDa to about 470 kDa, from about 440 kDa to about 480 kDa, from about 450 kDa to about 490 kDa, from about 460 kDa to about 500 kDa, or greater than 500 kDa.

In one embodiment, the silk preparation may include silk fibroin with a molecular weight of or an average molecular weight of 5-60 kDa.

In one embodiment, the silk preparation may include silk fibroin with a molecular weight of or an average molecular weight of 30-60 kDa. In one aspect, silk fibroin in this range maybe referred to as low molecular weight.

In one embodiment, the silk preparation may include silk fibroin with a molecular weight of or an average molecular weight of 100-300 kDa. In one aspect, silk fibroin in this range maybe referred to as high molecular weight.

In one embodiment, the silk preparation may include silk fibroin with a molecular weight of or an average molecular weight of 361 kDa.

Processed silk preparations may be analyzed, for example, by polyacrylamide gel electrophoresis (PAGE) alongside molecular weight standards to determine predominate molecular weights of proteins and/or polymers present. Additional methods for determining the molecular weight range or average molecular weight for a processed silk preparation may include, but are not limited to, sodium dodecyl sulfate (SDS)-PAGE, size-exclusion chromatography (SEC), high pressure liquid chromatography (HPLC), non-denaturing PAGE, and mass spectrometry (MS).

Processed silk preparations may include low molecular weight silk fibroin. As used herein, the term “low molecular weight silk fibroin” refers to silk fibroin with a molecular weight below 200 kDa. Some processed silk preparations may include high molecular weight silk fibroin. As used herein, the term “high molecular weight silk fibroin” refers to silk fibroin with a molecular weight equal to or greater than 200 kDa. In some embodiments, the silk fibroin molecular weight is defined by the degumming boiling time. In some embodiments, silk fibroin with a 480-minute boil, or “mb” may produce a to be low molecular weight silk fibroin when compared to a silk fibroin produced with a 120-minute boil, or “mb”. In some aspects, the 120 mb is considered to be high molecular weight silk fibroin in comparison to the 480 mb.

In some embodiments, silk fibroin molecular weight is modulated by the method of degumming used during processing. In some embodiments, longer heating times during degumming are used (e.g., see International Publication No. WO2014145002, the contents of which are herein incorporated by reference in their entirety). Longer heating (e.g., boiling) time may be used during the degumming process to prepare silk fibroin with lower average molecular weights. In some embodiments, heating times may be from about 1 min to about 5 min, from about 2 min to about 10 min, from about 5 min to about 15 min, from about 10 min to about 25 min, from about 20 min to about 35 min, from about 30 min to about 50 min, from about 45 min to about 75 min, from about 60 min to about 95 min, from about 90 min to about 125 min, from about 120 min to about 175 min, from about 150 min to about 200 min, from about 180 min to about 250 min, from about 210 min to about 350 min, from about 240 min to about 400 min, from about 270 min to about 450 min, from about 300 min to about 480 min, or more than 480 min. Additionally, the sodium carbonate concentration used in the degumming process, as well as the heating temperature, may also be altered to modulate the molecular weight of silk fibroin.

In some embodiments, silk fibroin molecular weight may be presumed, without actual analysis, based on methods used to prepare the silk fibroin. For example, silk fibroin may be presumed to be low molecular weight silk fibroin or high molecular weight silk fibroin based on the length of time that heating is carried out (e.g., by minute boil value).

In some embodiments, SBPs include a plurality of silk fibroin fragments generated using a dissociation procedure. The dissociation procedure may include one or more of heating, acid treatment, chaotropic agent treatment, sonication, and electrolysis. Some SBPs include a plurality of silk fibroin fragments dissociated from raw silk, silk fiber, and/or silk fibroin by heating. The heating may be carried out at a temperature of from about 30° C. to about 1,000° C. In some embodiments, heating is carried out by boiling. The raw silk, silk fiber, and/or silk fibroin may be boiled for from about 1 second to about 24 hours.

### Osmolarity

In some embodiments, SBP formulations may include processed silk with or without other components (e.g., excipients and cargo). The SBP formulations may have an osmotic concentration of from about 1 mOsm to about 10 mOsm, from about 2 mOsm to about 20 mOsm, from about 3 mOsm to about 30 mOsm, from about 4 mOsm to about 40 mOsm, from about 5 mOsm to about 50 mOsm, from about 6 mOsm to about 60 mOsm, from about 7 mOsm to about 70 mOsm, from about 8 mOsm to about 80 mOsm, from about 9 mOsm to about 90 mOsm, from about 10 mOsm to about 100 mOsm, from about 15 mOsm to about 150 mOsm, from about 25 mOsm to about 200 mOsm, from about 35 mOsm to about 250 mOsm, from about 45 mOsm to about 300 mOsm, from about 55 mOsm to about 350 mOsm, from about 65 mOsm to about 400 mOsm, from about 75 mOsm to about 450 mOsm, from about 85 mOsm to about 500 mOsm, from about 125 mOsm to about 600 mOsm, from about 175 mOsm to about 700 mOsm, from about 225 mOsm to about 800 mOsm, from about 275 mOsm to about 285 mOsm, from about 280 mOsm to about 900 mOsm, or from about 325 mOsm to about 1000 mOsm. The SBPs may have an osmolarity of from about 1 mOsm/L to about 10 mOsm/L, from about 2 mOsm/L to about 20 mOsm/L, from about 3 mOsm/L to about 30 mOsm/L, from about 4 mOsm/L to about 40 mOsm/L, from about 5 mOsm/L to about 50 mOsm/L, from about 6 mOsm/L to about 60 mOsm/L, from about 7 mOsm/L to about 70 mOsm/L, from about 8 mOsm/L to about 80 mOsm/L, from about 9 mOsm/L to about 90 mOsm/L, from about 10 mOsm/L to about 100 mOsm/L, from about 15 mOsm/L to about 150 mOsm/L, from about 25 mOsm/L to about 200 mOsm/L, from about 35 mOsm/L to about 250 mOsm/L, from about 45 mOsm/L to about 300 mOsm/L, from about 55 mOsm/L to about 350 mOsm/L, from about 65 mOsm/L to about 400 mOsm/L, from about 75 mOsm/L to about 450 mOsm/L, from about 85 mOsm/L to about 500 mOsm/L, from about 125 mOsm/L to about 600 mOsm/L, from about 175 mOsm/L to about 700 mOsm/L, from about 225 mOsm/L to about 800 mOsm/L, from about 275 mOsm/L to about 285 mOsm/L, from about 280 mOsm/L to about 900 mOsm/L, or from about 325 mOsm/L to about 1000 mOsm/L.

In some embodiment, the SBP formulation has an osmolarity from about 280-320 mOsm/L.

In some embodiment, the SBP formulation has an osmolarity from about 290-320 mOsm/L.

In some embodiment, the SBP formulation has an osmolarity of 280 mOsm/L.

In some embodiment, the SBP formulation has an osmolarity of 290 mOsm/L.

### Silk Fibroin Boiling Time

SBP formulations with processed silk with varying molecular weights. In some embodiments, the silk fibroin molecular weight is defined by the degumming boiling time. In some embodiments, silk fibroin with a 480-minute boil, or “mb” may produce be a low molecular weight silk fibroin when compared to a silk fibroin produced with a 120-minute boil, or “mb”. In some aspects, the 120 mb silk fibroin is considered to be high molecular weight silk fibroin in comparison to the 480 mb silk fibroin. In some embodiments, a longer boiling time is considered to be lower molecular weight silk fibroin. In some embodiments, a shorter boiling time is considered to be a higher molecular weight silk fibroin. In some embodiments, the boiling time is about 15 minutes, about 30 minutes, about 60 minutes, about 90 minutes, about 120 minutes, or about 480 minutes. In some embodiments, an SBP is prepared with processed silk with a single boiling time. In some embodiments, an SBP contains a blend of processed silk with different boiling times.

In one embodiment, the SBP formulation includes 30 mb silk fibroin.

In one embodiment, the SBP formulation includes 60 mb silk fibroin.

In one embodiment, the SBP formulation includes 90 mb silk fibroin.

In one embodiment, the SBP formulation includes 120 mb silk fibroin.

In one embodiment, the SBP formulation includes 480 mb silk fibroin.

### Purification and Concentration

In some embodiments, processed silk preparations may be purified. Purification, as used herein, refers to any process used to segregate or extract one entity from another. In some embodiments, purification is manual or automated. Purification may include the removal of salts, impurities, or contaminants from processed silk preparations.

In some embodiments, processed silk may be purified by concentration from a processed silk solution. Methods of concentrating silk fibroin from processed silk solutions may include any of those described in the International Publication No. WO2017139684, the contents of which are incorporated herein by reference in their entirety. In some embodiments, purification and/or concentration may be carried out by one or more of dialysis, centrifugation, air drying, vacuum drying, filtration, and/or Tangential Flow Filtration (TFF).

In some embodiments, processed silk solutions may be purified by dialysis. Dialysis may be carried out to remove undesired salts and/or contaminants. In some embodiments, processed silk solutions are concentrated via dialysis. Purification and/or concentration of processed silk by dialysis may be carried out as described in International Publication No. WO2005012606, the contents of which are herein incorporated by reference in their entirety. In some embodiments, the dialysis is performed against a hygroscopic polymer to concentrate the silk fibroin solution. In some embodiments the dialysis is manual, with the use of a membrane and manual solvent changes. In some embodiments, the solvent is changed between 1 and 10 times over the course of the procedure. In some embodiments, the membrane is a dialysis cassette. The dialysis cassette may be a slide-a-lyzer dialysis cassette. In some embodiments, the membrane is dialysis tubing. The dialysis tubing may be regenerated cellulose dialysis tubing and/or snake skin. The dialysis tubing or cassette may be rinsed in distilled water for 30 minutes to prepare the membrane for use. In some embodiments, the dialysis tubing has a molecular weight cutoff of 3.5 kDa. In some embodiments, the dialysis is performed at a temperature of from about 1° C. to about 30° C. In some embodiments, dialysis is performed at room temperature. In other embodiments, the dialysis is performed at 4° C. Dialysis may be performed until desired concentrations of silk fibroin and salt are obtained from processed silk solutions. Dialysis may be performed for periods of time from about 30 minutes to about 24 hours or beyond. For example, dialysis may be carried out for from about 30 minutes to about 2 hours, from about 1 hour to about 6 hours, from about 3 hours to about 10 hours, from about 5 hours, to about 12 hours, from about 7 hours to about 15 hours, from about 11 hours to about 20 hours, or from about 16 hours to about 24 hours.

In some embodiments, dialysis may be automated. The dialysis may use an automated water change system. Such systems may include tanks of up to 10 L and may be able to hold multiple dialysis cassettes (e.g., see International Publication No. WO2017106631, the contents of which are herein incorporated by reference in their entirety). Automated equipment may enable purification of larger volumes of solution with greater efficiency. Automated controllers, programmed with the proper times and volumes, may be used to facilitate changes of solvent or buffer over the course of dialysis. The solvent may be replaced from about 1 to about 20 times or more during dialysis. In some embodiments, automated dialysis may be completed in about 48 hours.

Dialysis may be performed with various solvents depending on the nature of the preparation being processed. In some embodiments the solvent may be water. In some embodiments, the solvent may be an aqueous solution. In some embodiments the solvent includes a hygroscopic polymer. Hygroscopic polymers may include, but are not limited to polyethylene glycol (PEG), polyethylene oxide (PEO), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, and polyanhydrides. Additional examples of hygroscopic polymers and related dialysis methods that may be employed include any of those found in International Publication Numbers WO2005012606, WO2005012606 and WO2017106631, and U.S. Pat. Nos. 6,302,848, 6,395,734, 6,127,143, 5,263,992, 6,379,690, 5,015,476, 4,806,355, 6,372,244, 6,310,188, 5,093,489, 6,325,810, 6,337,198, 6,267,776, 5,576,881, 6,245,537, 5,902,800, and 5,270,419, the contents of each of which are herein incorporated by reference in their entirety. Hygroscopic polymer concentrations may be from about 20% (w/v) to about 50% (w/v). In some embodiments, dialysis may be performed in a stepwise manner in a urea solution, and the urea solution may be subsequently be replaced with urea solutions of a lower concentration during buffer changes, until it is ultimately replaced with water, as described in Zheng et al. (2016) Journal of Biomaterials Applications 31:450-463.

In some embodiments, processed silk preparations may be purified by filtration. Such filtration may include trans flow filtration (TFF), also known as tangential flow filtration. During TFF, solutions may be passed across a filter membrane. Anything larger than the membrane pores would is retained, and anything smaller passes through the membrane (e.g., see International Publication No. WO2017106631, the contents of which are herein incorporated by reference in their entirety). With the positive pressure and flow along the membrane, instead of through it, particles trapped in the membrane may be washed away. TFF may be carried out using an instrument. The instrument may be automated. The membranes may be housed in TFF tubes with vertical inlets and outlets. The flow of solvent may be controlled by peristaltic pumps. Some TFF tubes may include a dual chamber element. The dual chamber element may enable TFF filtration of processed silk solutions at higher concentrations, while reducing aggregation via the reduction of shear forces.

In some embodiments, processed silk solutions are purified and/or concentrated by centrifugation. Centrifugation may be performed before or after other forms of purification, which include, but are not limited to dialysis and tangential flow filtration. Centrifugation times and speeds may be varied to optimize purification and/or concentration according to optimal time frames. Purification and/or concentration by centrifugation may include pelleting of the processed silk and removal of supernatant. In some cases, centrifugation is used to push solvent through a filter, while retaining processed silk. Centrifugation may be repeated as many times as needed. In some embodiments, silk fibroin solutions are centrifuged two or more times during concentration and/or purification.

In some embodiments, SBP formulations may be directly prepared from dialyzed silk fibroin. In some embodiments, SBP formulations may be directly prepared from dialyzed and filtered silk fibroin. In some embodiments, SBP formulations prepared from dialyzed silk fibroin, and optionally filtered, may be stored at 4° C. In some embodiments, SBP formulations prepared from dialyzed silk fibroin, may be frozen for storage. In some embodiments, SBP formulations prepared from dialyzed silk fibroin, may be frozen for storage and then thawed. These SBP formulations may maintain their physical properties after the freezing and thawing.

### Drying Methods

In some embodiments, processed silk preparations may be dried to remove solvent. In some embodiments, SBP formulations may be rinsed prior to drying. Methods of drying may include, but are not limited to, air drying, oven drying, lyophilization, spray drying, spray freezing, and vacuum drying. Drying may be carried out to alter the consistency and/or other properties of processed silk preparations. One or more compounds or excipients may be combined with processed silk preparations to improve processed silk recovery and/or reconstitution after the drying process. For example, sucrose may be added to improve silk fibroin recovery and reconstitution from dried solutions. In some embodiments, drying may be carried out in the fabrication of a processed silk format or a SBP. Examples include, but are not limited to fabrication of fibers, nanofibers, mats, films, foams, membranes, rods, tubes, gels, hydrogels, microspheres, nanospheres, solutions, patches, grafts and powders. In some embodiments, drying processed silk may be carried out by oven drying, lyophilizing, and/or air drying.

Oven drying refers to any drying method that uses an oven. According to some methods, ovens are maintained at temperatures of from about 30° C. to about 90° C. or more. In some embodiment, oven drying is carried out at a temperature of 60° C. Processed silk preparations may be placed in ovens for a period of from about 1 hour to about 24 hours or more. In one embodiment, SBP formulations are oven dried at 60° C. for 2 hours. Oven drying may be used to dry silk fibroin preparations. In some embodiments, silk fibroin preparations are oven dried for 16 hours at 60° C. to obtain a desired format. In some cases, silk fibroin solutions are oven dried overnight. Examples of formats obtained by oven drying may include, but are not limited to, fibers, nanofibers, mats, films, foams, membranes, rods, tubes, gels, hydrogels, microspheres, nanospheres, solutions, patches, grafts, and powders.

In some embodiments, processed silk preparations may be freeze dried. Freeze drying may be carried out by lyophilization. Freeze drying may require processed silk preparations to be frozen prior to freeze drying. Freezing may be carried out at temperatures of from about 5° C. and about −85° C. In some embodiments, freeze drying is carried out by lyophilization for up to 75 hours. In some embodiments, lyophilization is used to prepare processed silk formats or SBPs. Such formats may include, but are not limited to, fibers, nanofibers, mats, films, foams, membranes, rods, tubes, gels, hydrogels, microspheres, nanospheres, solutions, patches, grafts and powders. The use of lyophilization to fabricate SBPs may be carried out according to any of the methods described in Zhou et al. (2017) Acta Biomater S1742-7061(17)30569; Yang et al. (2017) Int J Nanomedicine 12:6721-6733; Seo et al. (2017) J Biomater Appl 32(4):484-491; Ruan et al. (2017) Biomed Pharmacother 97:600-606; Wu et al. (2017) J Mech Behav Biomed Mater 77:671-682; Zhao et al (2017) Materials Letters 211:110-113; Chen et al. (2017) PLoS One 12(11):e0187880; Min et al. (2017) Int J Biol Macromol 17: 32855-8; Sun et al. Journal of Materials Chemistry B 5:8770; and Thai et al. J Biomed Mater (2017) 13(1):015009, the contents of each of which are herein incorporated by reference in their entirety.

In some embodiments, processed silk preparations may be dried by air drying. “Air drying,” as used herein refers to the removal of moisture by exposure to ambient or circulated gasses. Air drying may include exposing a preparation to air at room temperature (from about 18° C. to about 29° C.). Air drying may be carried out for from about 30 minutes to about 24 hours or more. In some embodiments, silk fibroin preparations are air dried to prepare SBPs. SBP formats that may be prepared may include, but are not limited to, fibers, nanofibers, mats, films, foams, membranes, rods, tubes, gels, hydrogels, microspheres, nanospheres, solutions, patches, grafts and powders. Some examples of the use of air drying for fabrication of SBPs are presented in Susanin et al. (2017) Fibre Chemistry 49(2):88-96; Lo et al. J Tissue Eng Regen Med (2017) doi.10.1002/term.2616; and Mane et al. Scientific Reports 7:15531, the contents of each of which are herein incorporated by reference in their entirety.

### Spinning

In some embodiments, processed silk may be prepared by spinning. As used herein, the term “spinning” refers to a process of twisting materials together. Spinning may include the process of preparing a silk fiber by twisting silk proteins as they are secreted from silk producers. Other forms of spinning include spinning one or more forms of processed silk together to form a thread, filament, fiber, or yarn. The processed silk may already consist of a filamentous format prior to spinning. In some embodiments, processed silk is processed by spinning from a non-filamentous format (e.g., from a film, mat, or solution).

In some embodiments, spinning includes the technique of electrospinning. Electrospinning may be used to prepare silk fibers from silk fibroin. The silk fibroin may be dissolved in water or an aqueous solution before electrospinning. In other embodiments, silk fibroin is dissolved in an organic solvent before electrospinning. The organic solvent may be hexafluoroisopropanol (HFIP). In some embodiments, electrospinning may be carried out as described in Yu et al. (2017) Biomed Mater Res A doi. 10.1002/jbm.a.36297 or Chantawong et a. (2017) Mater Sci Mater Med 28(12):191, the contents of each of which are herein incorporated by reference in their entirety.

Electrospinning typically includes the use of an electrospinning apparatus. Processed silk may be added to the apparatus to produce silk fiber. The processed silk may be silk fibroin in solution. Electrospinning apparatus components may include one or more of a spinneret (also referred to as a spinnerette), needle, mandrel, power source, pump, and grounded collector. The apparatus may apply voltage to the dissolved silk fibroin, causing electrostatic repulsion that generates a charged liquid that is extruded from the end. Electrostatic repulsion also enables fiber elongation as it forms, and charged liquid cohesion prevents it from breaking apart. Resulting fiber may be deposited on the collector. In some embodiments, electrospinning methods may be carried out according to those described in European Patent No. EP3206725; Manchineella et al. (2017) European Journal of Organic Chemistry 30:43634369; Park et al. (2017) Int J Biomacromol S0141-8130(17):32645-4; Wang et al. (2017) J Biomed Mater Res A doi.10.1002/jbm.a.36225; Chendang et al. (2017) J Biomaterials and Tissue Engineering 7:858-862; Kambe et al. (2017) Materials (Basel) 10(10):E1153; Chouhan et al. (2017) J Tissue Eng Reneg Med doi.10.1002/term.2581; Genovese et al. (2017) ACS Appl Mater Interfaces doi.10.1021acsami.7b13372; Yu et al. (2017) Biomed Mater Res A doi. 10.1002/jbm.a.36297; Chantawong et al. (2017) Mater Sci Mater Med 28(12):191, the contents of each of which are herein incorporated by reference in their entirety.

In some embodiments, spinning may be carried out as dry spinning. Dry spinning may be carried out using a dry spinning apparatus. Dry spinning may be used to prepare silk fibers from processed silk preparations. The preparations may include silk fibroin solutions. The preparations may be aqueous solutions. Dry spinning apparatuses typically use hot air to dry processed silk as it is extruded. In some embodiments, dry spinning may be carried out according to any of the methods presented in Zhang et al. (2017) Int J Biol Macromol pii:S0141-8130(17):32857, the contents of which are herein incorporated by reference in their entirety.

### Spraying

In some embodiments, processing methods include spraying. As used herein, the term “spraying” refers to the sprinkling or showering of a compound or composition in the form of small drops or particles. Spraying may be used to prepare SBPs by spraying processed silk. Spraying may be carried out using electrospraying. Processed silk used for spraying may include processed silk in solution. The solution may be a silk fibroin solution. Solutions may be aqueous solutions. Some solutions may include organic solvents. Electrospraying may be carried out in a manner similar to that of electrospinning, except that the charged liquid lacks cohesive force necessary to prevent extruding material from breaking apart. In some embodiments, spraying methods may include any of those presented in United States Publication No. US2017/333351 or Cao et al. (2017) Scientific Reports 7:11913, the contents of each of which are herein incorporated by reference in their entirety. In some embodiments, electrospray methods include a coaxial system for coaxial spraying.

In some embodiments, spraying is carried out as spray drying. Spray drying is a method of producing a dry powder from a liquid or slurry by rapidly drying with a hot gas. For example, the silk fibroin solution may be introduced as a fine spray or mist into a tower or chamber with heated air. The large surface area of the spray droplets causes evaporation of the water to occur rapidly, converting the droplets into dry powder particles. The heat and drying process may induce beta-sheet formation in the silk fibroin. Additional advantages of spray drying may include low heat, speed, reproducibility, and scalability.

In one embodiment, the spraying is carried out as spray drying using the electrostatic spray drying methods known in the art.

In some embodiments, spraying is carried out as spray coating. For example, SBP formulations may be sprayed onto the surface of a substance to form a coating. The spray coating processing may be a thermal spray coating process where SBP formulations are heated or melted by a heat source, for example, by electrical means (plasma or arc) or chemical means (combustion flame). Thermal spraying techniques that may be used herein include, but are not limited to, plasma spraying, detonation spraying, wire arc spraying, flame spraying, high velocity oxy-fuel coating spraying (HVOF), high velocity air fuel (HVAF), warm spraying, and cold spraying.

In one embodiment, the spray coating may be used for enteric capsules.

### Precipitation

In some embodiments, processing methods include precipitation. As used herein, the term “precipitation” refers to the deposition of a substance in solid form from a solution. Precipitation may be used to obtain solid processed silk from processed silk solutions. The processed silk may be silk fibroin. Processed silk may be precipitate from a solution. The solvent may be aqueous. In some embodiments, the solvent is organic. Examples of organic solvents include, but are not limited to, HFIP, methanol, ethanol, and other alcohols. In some embodiments, the solvent is water. In some embodiments the solvent is a mixture of an organic solvent and water. Aqueous solvents may contain one or more salts. Processed silk may be precipitated from processed silk solutions by modulating one or more components of the solution to alter the solubility of the processed silk and promote precipitation. Additional processing steps may be employed to initiate or speed precipitation. Such methods may include, but are not limited to sonication, centrifugation, increasing the concentration of processed silk, altering the concentration of salt, adding additional salt or salts, altering the pH, applying shear stress, adding excipients, or applying chemical modifications.

### Processing Methods: Milling

In some embodiments, processing methods include milling. As used herein, “milling” generally refers to the process of breaking down a solid substance into smaller pieces using physical forces such as grinding, crushing, pressing and/or cutting. As a non-limiting example, SBP formulations may be milled to create powders. The density of powder formulations may be controlled during the milling process. As another non-limiting example, solid encapsulation of a therapeutic agent or cargo with another substance (e.g., SBPs) may be prepared by milling. The therapeutic agent or cargo may include any one of those described herein. In some embodiments, the therapeutic agent or cargo to be encapsulated by another substance may include SBPs.

### Altering Mechanical Properties

In some embodiments, the mechanical properties of processed silk may be altered by modulating physical and/or chemical properties of the processed silk. The mechanical properties include, but are not limited to, mechanical strength, tensile strength, elongation capabilities, elasticity, compressive strength, stiffness, shear strength, toughness, torsional stability, temperature stability, moisture stability, viscosity, and reeling rate. Examples of the physical and chemical properties used to tune the mechanical properties of processed silk include, but are not limited to, the temperature, formulations, silk concentration, β-sheet content, crosslinking, the molecular weight of the silk, the storage of the silk, storage, methods of preparation, dryness, methods of drying, purity, and degumming. Methods of tuning the mechanical strength of processed silk are taught in International Patent Application Publication No. WO2017123383, European Patent No. EP2904134, European Patent No. EP3212246, Fang et al., Wu et al., Susanin et al., Zhang et al., Jiang et al., Yu et al., Chantawong et al., and Zhang et al. (Fang et al. (2017) Journal of Materials Chemistry B 5(30):6042-6048; Wu et al. (2017) J Mech Behav Biomed Mater 77:671-682; Susanin et al. (2017) Fibre Chemistry 49(2):88-96; Zhang et al. (2017) Fibers and Polymers 203:9-16; Jiang et al. (2017) J Biomater Sci Polym Ed 15:1-36; Yu et a. (2017) Biomed Mater Res A doi. 10.1002/jbm.a.36297; Chantawong et al. (2017) Mater Sci Mater Med 28(12):191; Zhang et al. (2017) Int J Biomacromol S0141-8310(17):32857), the contents of each of which are herein incorporated by reference in their entirety.

In some embodiments, the excipients which may be incorporated in a formulation may be used to control the modulus of processed silk preparations. In some embodiments, these processed silk preparations are hydrogels. In some embodiments, processed silk hydrogels are prepared with different excipients and tested for their mechanical properties, including the modulus. Processed silk preparations may be assessed for modulus, shear storage modulus, shear loss modulus, phase angle, and viscosity using a rheometer, and/or any other method known to one skilled in the art. Rheometer geometry may be selected based on sample viscosity, shear rates, and shear stresses desired, as well as sample volumes. Geometries that are suitable for measuring the rheological properties of SBP formulations include, not are not limited to, cone and plate, parallel plates, concentric cylinders (or Bob and Cup), and double gap cylinders. In one embodiment, a cone and plate geometry is used. In another embodiment, a concentric cylinder geometry is used. Processed silk preparations may be tested both before and after gelation. In some embodiments, processed silk preparations are prepared, optionally with different excipients, and tested for their mechanical properties, including the shear storage modulus, the shear loss modulus, phase angle, and viscosity. As used herein, the term “shear storage modulus” refers to the measure of a material's elasticity or reversible deformation as determined by the material's stored energy. As used herein, the term “shear loss modulus” refer to the measure of a material's ability to dissipate energy, usually in the form of heat. As used herein, the term “phase angle” refers to the difference in the stress and strain applied to a material during the application of oscillating shear stress. As used herein, the term “viscosity” refers to a material's ability to resist deformation due to shear forces, and the ability of a fluid to resist flow. In some embodiments, processed silk hydrogels may possess similar viscosities, but vary in the modulus.

In some embodiments, the viscosity of SBPs is tunable between 1-1000 centipoise (cP). In some embodiments, the viscosity of an SBP is tunable from about 0.0001 to about 1000 Pascal seconds (Pa*s). In some embodiments, the viscosity of an SBP is from about 1 cP to about 10 cP, from about 2 cP to about 20 cP, from about 3 cP to about 30 cP, from about 4 cP to about 40 cP, from about 5 cP to about 50 cP, from about 6 cP to about 60 cP, from about 7 cP to about 70 cP, from about 8 cP to about 80 cP, from about 9 cP to about 90 cP, from about 10 cP to about 100 cP, from about 100 cP to about 150 cP, from about 150 cP to about 200 cP, from about 200 cP to about 250 cP, from about 250 cP to about 300 cP, from about 300 cP to about 350 cP, from about 350 cP to about 400 cP, from about 400 cP to about 450 cP, from about 450 cP to about 500 cP, from about 500 cP to about 600 cP, from about 550 cP to about 700 cP, from about 600 cP to about 800 cP, from about 650 cP to about 900 cP, or from about 700 cP to about 1000 cP. In some embodiments, the viscosity of an SBP is from about from about 0.0001 Pa*s to about 0.001 Pa*s, from about 0.001 Pa*s to about 0.01 Pa*s, from about 0.01 Pa*s to about 0.1 Pa*s, from about 0.1 Pa*s to about 1 Pa*s, from about 1 Pa*s to about 10 Pa*s, from about 2 Pa*s to about 20 Pa*s, from about 3 Pa*s to about 30 Pa*s, from about 4 Pa*s to about 40 Pa*s, from about 5 Pa*s to about 50 Pa*s, from about 6 Pa*s to about 60 Pa*s, from about 7 Pa*s to about 70 Pa*s, from about 8 Pa*s to about 80 Pa*s, from about 9 Pa*s to about 90 Pa*s, from about 10 Pa*s to about 100 Pa*s, from about 100 Pa*s to about 150 Pa*s, from about 150 Pa*s to about 200 Pa*s, from about 200 Pa*s to about 250 Pa*s, from about 250 Pa*s to about 300 Pa*s, from about 300 Pa*s to about 350 Pa*s, from about 350 Pa*s to about 400 Pa*s, from about 400 Pa*s to about 450 Pa*s, from about 450 Pa*s to about 500 Pa*s, from about 500 Pa*s to about 600 Pa*s, from about 550 Pa*s to about 700 Pa*s, from about 600 Pa*s to about 800 Pa*s, from about 650 Pa*s to about 900 Pa*s, from about 700 Pa*s to about 1000 Pa*s, or from about 10 Pa*s to about 2500 Pa*s.

In some embodiments, the shear storage modulus (G′) and/or the shear loss modulus (G″) is tunable from about 0.0001 to about 20000 Pascals (Pa). In some embodiments, G′ and/or G″ is from about 0.0001 Pa to about 0.001 Pa, from about 0.001 Pa to about 0.01 Pa, from about 0.01 Pa to about 0.1 Pa, from about 0.1 Pa to about 1 Pa, from about 1 Pa to about 10 Pa, from about 2 Pa to about 20 Pa, from about 3 Pa to about 30 Pa, from about 4 Pa to about 40 Pa, from about 5 Pa to about 50 Pa, from about 6 Pa to about 60 Pa, from about 7 Pa to about 70 Pa, from about 8 Pa to about 80 Pa, from about 9 Pa to about 90 Pa, from about 10 Pa to about 100 Pa, from about 100 Pa to about 150 Pa, from about 150 Pa to about 200 Pa, from about 200 Pa to about 250 Pa, from about 250 Pa to about 300 Pa, from about 300 Pa to about 350 Pa, from about 350 Pa to about 400 Pa, from about 400 Pa to about 450 Pa, from about 450 Pa to about 500 Pa, from about 500 Pa to about 600 Pa, from about 550 Pa to about 700 Pa, from about 600 Pa to about 800 Pa, from about 650 Pa to about 900 Pa, from about 700 Pa to about 1000 Pa, from about 1000 Pa to about 1500 Pa, from about 1500 Pa to about 2000 Pa, from about 2000 Pa to about 2500 Pa, from about 2500 Pa to about 3000 Pa, from about 3000 Pa to about 3500 Pa, from about 3500 Pa to about 4000 Pa, from about 4000 Pa to about 4500 Pa, from about 4500 Pa to about 5000 Pa, from about 5000 Pa to about 5500 Pa, from about 5500 Pa to about 6000 Pa, from about 6000 Pa to about 6500 Pa, from about 6500 Pa to about 7000 Pa, from about 7000 Pa to about 7500 Pa, from about 7500 Pa to about 8000 Pa, from about 8000 Pa to about 8500 Pa, from about 8500 Pa to about 9000 Pa, from about 9000 Pa to about 9500 Pa, from about 9500 Pa to about 10000 Pa, from about 10000 Pa to about 11000 Pa, from about 11000 Pa to about 12000 Pa, from about 12000 Pa to about 13000 Pa, from about 13000 Pa to about 14000 Pa, from about 14000 Pa to about 15000 Pa, from about 15000 Pa to about 16000 Pa, from about 16000 Pa to about 17000 Pa, from about 17000 Pa to about 18000 Pa, from about 18000 Pa to about 19000 Pa, or from about 19000 Pa to about 20000 Pa.

In some embodiments, the phase angle is tunable from about 0.0001° to about 90°. In some embodiments, the phase angle is from about 0.0001° to about 0.001°, from about 0.001° to about 0.01°, from about 0.01° to about 0.1°, from about 0.1° to about 1°, from about 1° to about 2°, from about 2° to about 3° from about 3° to about 4°, from about 4° to about 5°, from about 5° to about 6°, from about 6° to about 7°, from about 7° to about 8°, from about 8° to about 9°, from about 9° to about 10°, from about 10° to about 15°, from about 15° to about 20°, from about 20° to about 25, from about 25° to about 30°, from about 30° to about 35°, from about 35° to about 40, from about 40° to about 45°, from about 45° to about 50°, from about 50° to about 55° from about 55° to about 60° from about 60° to about 65° from about 65° to about 70°, from about 70° to about 75°, from about 75° to about 80°, from about 80° to about 85°, or from about 85° to about 90°.

In some embodiments, the concentration of processed silk may enable silk preparations to shear thin. In some embodiments the silk preparation is an SBP. In some embodiments, the SBP is a hydrogel. In some embodiments, the molecular weight of processed silk hydrogels may enable hydrogels to shear thin. In some embodiments, hydrogels prepared with low molecular weight silk fibroin may be injected with much less force than hydrogels of similar viscosity that are prepared with higher molecular weight silk fibroin. In some embodiments, hydrogels with low molecular weight silk fibroin display higher viscosity than hydrogels with high molecular weight silk fibroin.

### Modulating Degradation Resorption

In some embodiments, processed silks are or are processed to be biocompatible. As used herein, a “biocompatible” substance is any substance that is not harmful to most living organisms or tissues. With some processed silk, degradation may result in products that are biocompatible, making such processed silk attractive for a variety of applications. Some processed silk may degrade into smaller proteins or amino acids. Some processed silk may be resorbable under physiological conditions. In some embodiments, products of silk degradation may be resorbable in vivo. In some embodiments, the rate of degradation of processed silk may be tuned by altering processed silk properties. Examples of these properties include, but are not limited to, type and concentration of certain proteins, 3-sheet content, crosslinking, silk fibroin molecular weight, and purity. In some embodiments, rate of processed silk degradation may be modulated by method of storage, methods of preparation, dryness, methods of drying, reeling rate, and degumming process.

In some embodiments, the bioresorbability and degradation of processed silk is modulated by the addition of sucrose, as taught in Li et al. (Li et al. (2017) Biomacromolecules 18(9):2900-2905), the contents of which are herein incorporated by reference in their entirety. Processed silk may be formulated with sucrose to enhance thermal stability. Furthermore, processed silk with sucrose may also be formulated with antiplasticizing agents to further enhance thermal stability of processed silk, SBPs, and/or therapeutic agents included in SBPs. Methods of increasing thermal stability using antiplasticizing agents may include any of those described in Li et al. (Li et al. (2017) Biomacromolecules 18(9):2900-2905), the contents of which are herein incorporated by reference in their entirety. In some embodiments, the addition of sucrose to processed silk preparations prior to lyophilization leads to an increased reconstitution efficiency. In some embodiments, the addition of sucrose may be used to create higher molecular weight processed silk preparations as well as to maintain long term storage stability. In some embodiments, the incorporation of sucrose into processed silk preparations described herein enables slower freezing during lyophilization cycle.

In some embodiments, the bioresorbability and degradation of processed silk may be tuned through formulation with additional bioresorbable polymer matrices, as taught in International Publication Numbers WO2017177281 and WO2017179069, the contents of each of which are herein incorporated by reference in their entirety. In some embodiments, the polymer matrix is polyurethane. In some embodiments, these polymer matrices may be polycaprolactone and a ceramic filler. The ceramic filler may include MgO.

In some embodiments, the bioresorbability and degradation of processed silk is tuned through the fabrication of a composite scaffold. Composite scaffolds, combinations of scaffolds or scaffolds formed from more than one material, may be formed from two or more processed silk preparations. In some embodiments, processed silk scaffolds comprising a combination of silk fibroin microspheres within a larger processed silk preparation may demonstrate slower degradation in comparison with other scaffolds, as taught in European Patent No. EP3242967, the contents of which are herein incorporated by reference in their entirety.

### Analytics

In some embodiments, processed silk products may be analyzed for properties such as molecular weight, aggregation, amino acid content, lithium content, and endotoxin level. Such properties may be evaluated via any analytical methods known in the art. As a non-limiting example, the Ultra-Performance Liquid Chromatography (UPLC)-Size Exclusion Chromatography (SEC) method may be used to assess the molecular weight and/or aggregation of the silk fibroin proteins in the processed silk products.

In some embodiments, processed silk products may be analyzed for silk fibroin concentration. Such properties may be evaluated via any analytical methods known in the art. As a non-limiting example, gravimetry and/or ultraviolet-visible spectroscopy (UV-Vis) may be used.

### Residence Time

In some embodiments, SBP formulations may be prepared to have desired residence time according to the application for which they are designed. As used herein, the term “residence time” refers to the average length of time during which a substance (e.g., SBP formulations) is in a given location or condition. In some embodiments, residence time of SBP formulations described herein may vary from a few hours to several months. For example, residence time of SBP formulations may be about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, or longer than 1 year.

### Excipients

In some embodiments, SBPs include one or more excipients. In some embodiments, SBP formulation may not include an excipient. As used herein, the term “excipient” refers to any substance included in a composition with an active agent or primary component, often serving as a carrier, diluent, or vehicle for the active agent or primary component. In some embodiments, excipients may be compounds or compositions approved for use by the US Food and Drug Administration (FDA). In some embodiments, SBPs may include excipients that increase SBP stability or stability of one or more other SBP components. Some SBPs may include an excipient that modulates payload release. Excipients may include, but are not limited to, solvents, diluents, liquid vehicles, dispersion or suspension media or aids, surfactants, thickening agents, emulsifying agents, lipids, liposomes, isotonic agents, buffers, gelation agents and preservatives. In some embodiments, excipients include lipidoids, lipid nanoparticles, polymers, lipoplexes, particles, core-shell nanoparticles, peptides, proteins, cells, hyaluronidase, and/or nanoparticle mimics. In some embodiments, processed silk and/or SBPs may be used as an excipient. In some embodiments, excipients included in SBPs are selected from one or more of those listed in Table 1. In the Table, example categories are indicated for each excipient. These categories are not limiting and each excipient may fall under multiple categories (e.g., any of the categories of excipients described herein).

TABLE 1
Excipients

| Excipient | Example Category |
|---|---|
| Avicel | bulking agent |
| bulking agent | bulking agent |
| copolymers of vinylpyrrolidone and vinylacetate | bulking agent |
| dibasic calcium phosphate dehydrate | bulking agent |
| fumaric acid | bulking agent |
| hydroxypropylmethylcellulose | bulking agent |
| lactose USP | bulking agent |
| malic acid | bulking agent |
| microcrystalline cellulose | bulking agent |
| polyvinylpyrrolidone | bulking agent |
| tartaric acid | bulking agent |
| (12Z,15Z)-N,N-dimethyl-2-nonylhenicosa-12,15-dien-1-amine | cationic lipid |
| (12Z,15Z)-N,N-dimethylhenicosa-12,15-dien-4-amine | cationic lipid |
| (13Z,16Z)-N,N-dimethyl-3-nonyldocosa-13,16-dien-1-amine | cationic lipid |
| (13Z,16Z)-N,N-dimethyldocosa-13,16-dien-5-amine | cationic lipid |
| (14Z)-N,N-dimethylnonacos-14-en-10-amine | cationic lipid |
| (14Z,17Z)-N,N-dimethyltricosa-14,17-dien-4-amine | cationic lipid |
| (14Z,17Z)-N,N-dimethyltricosa-14,17-dien-6-amine | cationic lipid |
| (15Z)-N,N-dimethyleptacos-15-en-10-amine | cationic lipid |
| (15Z,18Z)-N,N-dimethyltetracosa-15,18-dien-7-amine | cationic lipid |
| (15Z,18Z)-N,N-dimethyltetracosa-15,18-dien-5-amine | cationic lipid |
| (16Z)-N,N-dimethylpentacos-16-en-8-amine | cationic lipid |
| (16Z,19Z)-N,N-dimethylpentacosa-16,19-dien-6-amine | cationic lipid |
| (17Z)-N,N-dimethylhexacos-17-en-9-amine | cationic lipid |
| (17Z)-N,N-dimethylnonacos-17-en-10-amine | cationic lipid |
| (17Z,20Z)-N,N -dimemylhexacosa-17,20-dien-9-amine | cationic lipid |
| (17Z,20Z)-N,N-dimethylhexacosa-17,20-dien-7-amine | cationic lipid |
| (18Z)-N,N-dimetylheptacos-18-en-10-amine | cationic lipid |
| (18Z,21Z)-N,N-dimethylheptacosa-18,21-dien-8-amine | cationic lipid |
| (18Z,21Z)-N,N-dimethylheptacosa-18,21-dien-10-amine | cationic lipid |
| (19Z,22Z)-N,N-dimeihyloctacosa-19,22-dien-9-amine | cationic lipid |
| (19Z,22Z)-N,N-dimethyloctacosa-19,22-dien-7-amine | cationic lipid |
| (11E,20Z,23Z)-N,N-dimethylnonacosa-11,20,2-trien-10-amine | cationic lipid |
| (1Z,19Z)-N5N-dimethylpentacosa-16,19-dien-8-amine | cationic lipid |
| (20Z)-N,N-dimethylheptacos-20-en-10-amine | cationic lipid |
| (20Z)-N,N-dimethylnonacos-20-en-10-amine | cationic lipid |
| (20Z,23Z)-N,N-dimethylnonacosa-20,23-dien-10-amine | cationic lipid |
| (20Z,23Z)-N-ethyl-N-methylnonacosa-20,23-dien-10-amine | cationic lipid |
| (21Z,24Z)-N,N-dimethyltriaconta-21,24-dien-9-amine | cationic lipid |
| (22Z)-N,N-dimethylhentriacont-22-en-10-amine | cationic lipid |
| (22Z,25Z)-N,N-dimethylhentriaconta-22,25-dien-10-amine | cationic lipid |
| (24Z)-N,N-dimethyltritriacont-24-en-10-amine | cationic lipid |
| (2R)-1-[(3,7-dimethyloctyl)oxy]-N,N-dimethyl-3-[(9Z,12Z)-octadeca- | cationic lipid |
| 9,12-dien-1-yloxy]propan-2-amine | |
| (2R)-N,N-dimethyl-H(1-metoyloctyl)oxy]-3-[(9Z,12Z)-octadeca- | cationic lipid |
| 9,12-dien-1-yloxy]propan-2-amine | |
| (2S)-1-(heptyloxy)-N,N-dimethyl-3-[(9Z,12Z)-octadeca-9,12-dien-1- | cationic lipid |
| yloxy]propan-2-amine | |
| (2S)-1-(hexyloxy)-3-[(11Z,14Z)-icosa-11,14-dien-1-yloxy]-N,N- | cationic lipid |
| dimethylpropan-2-amine | |
| (2S)-1-(hexyloxy)-N,N-dimethyl-3-[(9Z,12Z)-octadeca-9,12-dien-1- | cationic lipid |
| yloxy]propan-2-amine | |
| (2S)-1-[(11Z,14Z)-icosa-11,14-dien-1-yloxy]-N,N-dimethyl-3- | cationic lipid |
| (pentyloxy)propan-2-amine | |
| (2S)-1-[(13Z)-docos-13-en-1-yloxy]-3-(hexyloxy)-N,N- | cationic lipid |
| dimethylpropan-2-amine | |
| (2S)-1-[(13Z,16Z)-docosa-13,16-dien-1-yloxy]-3-(hexyloxy)-N,N- | cationic lipid |
| dimethylpropan-2-amine | |
| (2S)-N,N-dimethyl-1-[(6Z,9Z,12Z)-octadeca-6,9,12-trien-1-yloxy]-3- | cationic lipid |
| (octyloxy)propan-2-amine | |
| (2S)-N,N-dimethyl-1-[(9Z,12Z)-octadeca-9,12-dien-1-yloxy]-3-[(5Z)- | cationic lipid |
| oct-5-en-1-yloxy]propan-2-amine | |
| 1,2-dilinolenyloxy-3-dimethylaminopropane (DLenDMA) | cationic lipid |
| 1,2-distearloxy-N,N-dimethylaminopropane (DSDMA) | cationic lipid |
| 1-[(11Z,14Z)-icosa-11,14-dien-1-yloxy]-N,N-dimethyl-3- | cationic lipid |
| (octyloxy)propan-2-amine | |
| 1-[(11Z,14Z)-1-nonylcosa-11,14-dien-1-yl] pyrrolidine | cationic lipid |
| 1-[(13Z)-docos-13-en-1-yloxy]-N,N-dimethyl-3-(octyloxy)propan-2- | cationic lipid |
| amine | |
| 1-[(13Z,16Z)-docosa-13,16-dien-1-yloxy]-N,N-dimethyl-3- | cationic lipid |
| (octyloxy)propan-2-amine | |
| 1-[(1R,2S)-2-heptylcyclopropyl]-N,N-dimethyloctadecan-9-amine | cationic lipid |
| 1-[(1S,2R)-2-decylcyclopropyl]-N,N-dimethylpentadecan-6-amine | cationic lipid |
| 1-[(1S,2R)-2-hexylcyclopropyl]-N,N-dimethylnonadecan-10-amine | cationic lipid |
| 1-[(9Z)-hexadec-9-en-1-yloxy]-N,N-dimethyl-3-(octyloxy)propan-2- | cationic lipid |
| amine | |
| 1-{2-[(9Z,12Z)-octadeca-9,12-dien-1-yloxy]-1- | cationic lipid |
| [(octyloxy)methyl]ethyl}azetidine | |
| 1-{2-[(9Z,12Z)-octadeca-9,12-dien-1-yloxy]-1- | cationic lipid |
| [(octyloxy)methyl]ethyl}pyrrolidine | |
| cationic lipid | cationic lipid |
| CLI-CLXXIX of International Publication No. WO2008103276 | cationic lipid |
| DLin-DMA | cationic lipid |
| DODMA | cationic lipid |
| formula CLI-CLXXIX of U.S. Pat. No. 7,893,302 | cationic lipid |
| formula CLI-CLXXXXII of U.S. Pat. No. 7,404,969 | cationic lipid |
| formula I-VI of United States Patent Publication No. US20100036115 | cationic lipid |
| N,N-dimethyl-1-(octyloxy)-3-({8-[(1S,2S)-2-{[(1R,2R)-2- | cationic lipid |
| penlylcyclopropyl]methyl}cyclopropyl]octyl}oxy)propan-2-amine | |
| N,N-dimethyl-1-{[8-(2-oclylcyclopropyl)octyl]oxy}-3- | cationic lipid |
| (octyloxy)propan-2-amine | |
| N,N-dimethyl-21-[(1S,2R)-2-octylcyclopropyl]henicosan-10-amine | cationic lipid |
| N,N-dimethyl-3-{7-[(1S,2R)-2-octylcyclopropyl]heptyl} dodecan-1- | cationic lipid |
| amine | |
| N,N-dimethylhepiacosan-10-amine | cationic lipid |
| N,N-dimethyl-1-[(1S,2R)-2-octylcyclopropyl] eptadecan-8-amine | cationic lipid |
| N,N-dimethyl-1-[(1S,2R)-2-octylcyclopropyl]pentadecan-8-amine | cationic lipid |
| R-N,N-dimethyl-1-[(9Z,12Z)-octadeca-9,12-dien-1-yloxy]-3- | cationic lipid |
| (octyloxy)propan-2-amine | |
| S-N,N-dimethyl-1-[(9Z,12Z)-octadeca-9,12-dien-1-yloxy]-3- | cationic lipid |
| (octyloxy)propan-2-amine | |
| N,N-dimethyl-[(1R,2S)-2-undecylcyclopropyl]tetradecan-5-amine | cationic lipid |
| N,N-dimethyl-1-(nonyloxy)-3-[(9Z,12Z)-octadeca-9,12-dien-1- | cationic lipid |
| yloxy]propan-2-amine | |
| N,N-dimethyl-1-[(1S,2R)-2-octylcyclopropyl]nonadecan-10-amine | cationic lipid |
| N,N-dimethyl-1-[(1S,2R)-2-octylcyclopropyl]hexadecan-8-amine | cationic lipid |
| N,N-dimethyl-1-[(1S,2S)-2-{[(1R,2R)-2- | cationic lipid |
| pentylcyclopropyl]methyl}cyclopropyl]nonadecan-10-amine | |
| N,N-dimethyl-1-[(9Z)-octadec-9-en-1-yloxy]-3-(octyloxy)propan-2- | cationic lipid |
| amine | |
| coating agents | coating agent |
| poly(alkyl)(meth)acrylate | coating agent |
| poly(ethylene-co-vinyl acetate) | coating agent |
| zein | coating agent |
| apocarotenal | colorant |
| apocarotenal derivative | colorant |
| astaxanthin | colorant |
| astaxanthin derivative | colorant |
| bixin | colorant |
| canthaxanthin | colorant |
| canthaxanthin derivative | colorant |
| capsanthin | colorant |
| capsanthin derivative | colorant |
| capsorubin derivative | colorant |
| capsorubin occurring in paprika ole-oresin | colorant |
| caretinoids | colorant |
| colorant | colorant |
| crocin | colorant |
| crocin derivative | colorant |
| dyes | colorant |
| FD&C Blue No. 2 (indigotine) | colorant |
| FD&C colorant | colorant |
| FD&C Red No. 3 (erythrosine) | colorant |
| FD&C Red No. 40 (allura red AC) | colorant |
| food coloring | colorant |
| inks | colorant |
| lutein | colorant |
| lutein derivative | colorant |
| lycopene | colorant |
| pigments | colorant |
| rhodoxanthin | colorant |
| rubixanthin | colorant |
| saffron | colorant |
| saffron derivative | colorant |
| turmeric | colorant |
| violaxanthin | colorant |
| β-carotene | colorant |
| β-carotene derivative | colorant |
| flowability agents | flowability agent |
| 1-dodecylazacyclo-heptan-2-one | gelling agent |
| 2-pyrrolidone | gelling agent |
| acacia | gelling agent |
| alginic acid | gelling agent |
| alpha-cyclodextrin | gelling agent |
| beeswax | gelling agent |
| bentonite | gelling agent |
| benzyl alcohol | gelling agent |
| beta-cyclodextrin | gelling agent |
| caprolactam | gelling agent |
| CARBOPOL ® (also known as carbomer) | gelling agent |
| carboxymethyl cellulose | gelling agent |
| castor oil | gelling agent |
| corn oil | gelling agent |
| cottonseed oil | gelling agent |
| cremaphor RH 40 | gelling agent |
| cremaphor RH 60 | gelling agent |
| d-alpha-tocopherol | gelling agent |
| di-fatty acid ester of PEG 1750 | gelling agent |
| di-fatty acid ester of PEG 300 | gelling agent |
| di-fatty acid ester of PEG 400 | gelling agent |
| dimethyl sulfoxide | gelling agent |
| dimethylacetamide (DMA) | gelling agent |
| dimethylformamide | gelling agent |
| distearoylphosphatidylglycerol | gelling agent |
| ethanol | gelling agent |
| ethyl acetate | gelling agent |
| ethylcellulose | gelling agent |
| gamma-cyclodextrin | gelling agent |
| gelatin | gelling agent |
| Gellucire 44/14 | gelling agent |
| glycerin | gelling agent |
| glycerol | gelling agent |
| glycerol formal | gelling agent |
| glycerophosphate | gelling agent |
| hydrogenated soy phosphatidylcholine | gelling agent |
| hydrogenated soybean oil | gelling agent |
| hydrogenated vegetable oils | gelling agent |
| hydroxy ethyl cellulose | gelling agent |
| hydroxyethyl cellulose | gelling agent |
| hydroxypropyl beta-cyclodextrin | gelling agent |
| hydroxypropyl cellulose | gelling agent |
| hydroxypropyl-beta-cyclodextrin | gelling agent |
| kolliphor 124 | gelling agent |
| kolliphor 181 | gelling agent |
| kolliphor 188 | gelling agent |
| kolliphor 407 | gelling agent |
| kolliphor EL (cremaphor EL) | gelling agent |
| kolliphor RH60 | gelling agent |
| Labrafil M-1944CS | gelling agent |
| Labrafil M-2125CS | gelling agent |
| Labrasol | gelling agent |
| L-alpha-dimyristoylphosphatidylcholine | gelling agent |
| L-alphadimyristoylphosphatidylglycerol | gelling agent |
| magnesium aluminum silicate | gelling agent |
| medium chain triglyceride | gelling agent |
| medium-chain diglyceride | gelling agent |
| medium-chain mono-glyceride | gelling agent |
| medium-chain triglyceride of coconut oil | gelling agent |
| medium-chain triglyceride of palm seed oil | gelling agent |
| methyl acetate | gelling agent |
| methylcellulose | gelling agent |
| mono-fatty acid ester of PEG 1750 | gelling agent |
| mono-fatty acid ester of PEG 300 | gelling agent |
| mono-fatty acid ester of PEG 400 | gelling agent |
| N-methyl-2-pyrrolidone | gelling agent |
| oleic acid | gelling agent |
| olive oil | gelling agent |
| peanut oil | gelling agent |
| PEG 1000 succinate | gelling agent |
| PEG 1750 | gelling agent |
| PEG 300 | gelling agent |
| PEG 300 caprylic/capric glyceride (Softigen 767) | gelling agent |
| PEG 300 linoleic glyceride (Labrafil M-2125CS) | gelling agent |
| PEG 300 oleic glyceride (Labrafil M-1944CS) | gelling agent |
| PEG 400 | gelling agent |
| PEG 400 caprylic/capric glyceride (Labrasol) | gelling agent |
| PEG 4000 (PEG 4 kDa) | gelling agent |
| peppermint oil | gelling agent |
| polaxamer | gelling agent |
| poloxamer-188 | gelling agent |
| poloxamer-407 | gelling agent |
| polyoxyl 40 stearate (PEG 1750 monosterate) | gelling agent |
| polyoxyl 8 stearate (PEG 400 monosterate) | gelling agent |
| polysorbate 20 | gelling agent |
| polysorbate-80 (Tween ®-80) | gelling agent |
| polysorbate-SO | gelling agent |
| polyvinyl alcohol | gelling agent |
| polyvinyl pyrrolidone | gelling agent |
| polyvinyl pyrrolidone-12 | gelling agent |
| polyvinyl pyrrolidone-17 | gelling agent |
| propylene carbonate | gelling agent |
| propylene glycol | gelling agent |
| safflower oil | gelling agent |
| sesame oil | gelling agent |
| sodium alginate | gelling agent |
| Softigen 767 | gelling agent |
| solutol HS 15 | gelling agent |
| sorbitan monooleate | gelling agent |
| sorbitan monooleate (Span 20) | gelling agent |
| sorbitol | gelling agent |
| soybean oil | gelling agent |
| sulfobutylether-beta-cyclodextrin | gelling agent |
| sulfo-butylether-beta-cyclodextrin | gelling agent |
| tetrahydrofuran | gelling agent |
| tragacanth | gelling agent |
| transcutol | gelling agent |
| triacetin | gelling agent |
| triethanolamine | gelling agent |
| triethylamine | gelling agent |
| xanthan gum | gelling agent |
| (50:50, Poly(D1-Lactic-Co-Glycolic Acid) | general |
| (50:50, Polyacrylic Acid (250000 Mw) | general |
| 1,2,6-Hexanetriol | general |
| 1,2-Dimyristoyl-Sn-Glycero-3-(Phospho-S-(1-Glycerol)) | general |
| 1,2-Dimyristoyl-Sn-Glycero-3-Phosphocholine | general |
| 1,2-Dioleoyl-Sn-Glycero-3-Phosphocholine | general |
| 1,2-Dipalmitoyl-Sn-Glycero-3-(Phospho-Rac-(1-Glycerol)) | general |
| 1,2-Distearoyl-Sn-Glycero-3-(Phospho-Rac-(1-Glycerol)) | general |
| 1,2-Distearoyl-Sn-Glycero-3-Phosphocholine | general |
| 1-O-Tolylbiguanide | general |
| 2-Ethyl-1,6-Hexanediol | general |
| Acetic Acid | general |
| Acetic Anhydride | general |
| Acetone | general |
| Acetone Sodium Bisulfite | general |
| Acetylated Lanolin Alcohol | general |
| Acetylated Monoglyceride | general |
| Acetylcysteine | general |
| Acetyltry ptophan (DL-) | general |
| Acrylates Copolymer | general |
| Acrylic Acid-Isooctyl Acrylate Copolymer | general |
| Acrylic Adhesive 788 | general |
| Activated Charcoal | general |
| Adcote 72A103 | general |
| Adhesive Tape | general |
| Adipic Acid | general |
| Aerotex Resin 3730 | general |
| Alanine | general |
| albumin | general |
| Albumin Aggregated | general |
| Albumin Colloidal | general |
| Albumin Human | general |
| Alcohol | general |
| Alfadex | general |
| Alkyl Ammonium Sulfonic Acid Betaine | general |
| Alkyl Aryl Sodium Sulfonate | general |
| Allantoin | general |
| Allyl Alpha-Ionone | general |
| Almond Oil | general |
| Alpha Terpineol | general |
| Alpha-Tocopherol (DL-) | general |
| Alpha-Tocopherol Acetate (DL-) | general |
| Aluminum Acetate | general |
| Aluminum Chlorhydroxy Allantoinate | general |
| Aluminum Hydroxide | general |
| Aluminum Hydroxide - Sucrose | general |
| Aluminum Hydroxide Gel | general |
| Aluminum Hydroxide Gel F 500 | general |
| Aluminum Hydroxide Gel F 5000 | general |
| Aluminum Monostearate | general |
| Aluminum Oxide | general |
| Aluminum Polyester | general |
| Aluminum Silicate | general |
| Aluminum Starch Octenylsuccinate | general |
| Aluminum Stearate | general |
| Aluminum Subacetate | general |
| Aluminum Sulfate Anhydrous | general |
| Amerchol C | general |
| Amerchol-Cab | general |
| Aminomethylpropanol | general |
| Ammonia | general |
| Ammonia Solution | general |
| Ammonium Acetate | general |
| Ammonium Hydroxide | general |
| Ammonium Lauryl Sulfate | general |
| Ammonium Nonoxynol-4 Sulfate | general |
| Ammonium Salt Of C-12-C-15 Linear Primary Alcohol Ethoxylate | general |
| Ammonium Sulfate | general |
| Ammonyx | general |
| Amphoteric-2 | general |
| Amphoteric-9 | general |
| Anethole | general |
| Anhydrous Citric Acid | general |
| Anhydrous Dextrose | general |
| Anhydrous Lactose | general |
| Anhydrous Trisodium Citrate | general |
| Aniseed Oil | general |
| Anoxid Sbn | general |
| Antifoam | general |
| Antipyrine | general |
| Apaflurane | general |
| Apricot Kernel Oil Peg-6 Esters | general |
| Aquaphor | general |
| Arginine | general |
| Arlacel | general |
| Ascorbic Acid | general |
| Ascorbyl Palmitate | general |
| Aspartic Acid | general |
| Bacteriostatic | general |
| Balsam Peru | general |
| Barium Sulfate | general |
| Beheneth-10 | general |
| Benzalkonium Chloride | general |
| Benzenesulfonic Acid | general |
| Benzethonium Chloride | general |
| Benzododecinium Bromide | general |
| Benzoic Acid | general |
| Benzyl Benzoate | general |
| Benzyl Chloride | general |
| Betadex | general |
| Bibapcitide | general |
| Bismuth Subgallate | general |
| Boric Acid | general |
| Brocrinat | general |
| Butane | general |
| Butyl Alcohol | general |
| Butyl Ester Of Vinyl Methyl Ether/Maleic Anhydride Copolymer | general |
| (125000 Mw) | |
| Butyl Stearate | general |
| Butylated Hydroxyanisole | general |
| Butylated Hydroxytoluene | general |
| Butylene Glycol | general |
| Butylparaben | general |
| Butyric Acid | general |
| C20-40 Pareth-24 | general |
| Caffeine | general |
| Calcium | general |
| Calcium Carbonate | general |
| Calcium Chloride | general |
| Calcium Gluceptate | general |
| Calcium Hydroxide | general |
| Calcium Lactate | general |
| Calcobutrol | general |
| Caldiamide Sodium | general |
| Caloxetate Trisodium | general |
| Calteridol Calcium | general |
| Canada Balsam | general |
| Caprylic/Capric Triglyceride | general |
| Caprylic/Capric/Stearic Triglyceride | general |
| Captan | general |
| Captisol | general |
| Caramel | general |
| Carbomer 1342 | general |
| Carbomer 1382 | general |
| Carbomer 934 | general |
| Carbomer 934p | general |
| Carbomer 940 | general |
| Carbomer 941 | general |
| Carbomer 980 | general |
| Carbomer 981 | general |
| Carbomer Homopolymer Type B (Allyl Pentaerythritol Crosslinked) | general |
| Carbomer Homopolymer Type C (Allyl Pentaerythritol Crosslinked) | general |
| Carbon Dioxide | general |
| Carboxy Vinyl Copolymer | general |
| Carboxymethylcellulose (CMC) | general |
| Carboxymethylcellulose Sodium | general |
| Carboxypolymethylene | general |
| Carrageenan | general |
| Carrageenan Salt | general |
| Cedar Leaf Oil | general |
| Cellobiose | general |
| Cellulose | general |
| Cerasynt-Se | general |
| Ceresin | general |
| Ceteareth-12 | general |
| Ceteareth-15 | general |
| Ceteareth-30 | general |
| Cetearyl Alcohol/Ceteareth-20 | general |
| Cetearyl Ethylhexanoate | general |
| Ceteth-10 | general |
| Ceteth-2 | general |
| Ceteth-20 | general |
| Ceteth-23 | general |
| Cetostearyl Alcohol | general |
| Cetrimonium Chloride | general |
| Cetyl Alcohol | general |
| Cetyl Esters Wax | general |
| Cetyl Palmitate | general |
| Cetylpyridinium Chloride | general |
| Chlorobutanol | general |
| Chlorobutanol Hemihydrate | general |
| Chlorocresol | general |
| Chloroutanol anhydrous | general |
| Chloroxylenol | general |
| Cholesterol | general |
| Choleth | general |
| Choleth-24 | general |
| Citrate | general |
| Citric Acid | general |
| citric acid (hydrous) | general |
| Citric Acid Monohydrate | general |
| Cocamide Ether Sulfate | general |
| Cocamine Oxide | general |
| Coco Betaine | general |
| Coco Diethanolamide | general |
| Coco Monoethanolamide | general |
| Cocoa Butter | general |
| Coco-Glycerides | general |
| Coconut Oil | general |
| Coconut Oil glycerides | general |
| Cocoyl Caprylocaprate | general |
| Cola Nitida Seed Extract | general |
| Collagen | general |
| Colloidal | general |
| Coloring Suspension | general |
| Corn | general |
| Cream Base | general |
| Creatine | general |
| Creatinine | general |
| Cresol | general |
| Croscarmellose Sodium | general |
| Crospovidone | general |
| Cupric Sulfate | general |
| Cupric Sulfate Anhydrous | general |
| Cyclomethicone | general |
| Cyclomethicone/Dimethicone Copolyol | general |
| Cysteine | general |
| Cysteine (DL-) | general |
| Cysteine Hydrochloride | general |
| Cysteine Hydrochloride Anhydrous | general |
| D&C Red No. 28 | general |
| D&C Red No. 33 | general |
| D&C Red No. 36 | general |
| D&C Red No. 39 | general |
| D&C Yellow No. 10 | general |
| Dalfampridine | general |
| Daubert 1-5 Pestr (Matte) 164z | general |
| Decyl Methyl Sulfoxide | general |
| Dehydag Wax Sx | general |
| Dehydrated | general |
| Dehydroacetic Acid | general |
| Dehymuls E | general |
| Denatonium Benzoate | general |
| Denatured | general |
| Dental | general |
| Deoxycholic Acid | general |
| Dextran | general |
| Dextran 40 | general |
| Dextrin | general |
| Dextrose | general |
| Dextrose Monohydrate | general |
| Dextrose Solution | general |
| Diatrizoic Acid | general |
| Diazolidinyl Urea | general |
| Dichlorobenzyl Alcohol | general |
| Dichlorodifluoromethane | general |
| Dichlorotetrafluoroethane | general |
| Diethanolamine | general |
| Diethyl Pyrocarbonate | general |
| Diethyl Sebacate | general |
| Diethylene Glycol Monoethyl Ether | general |
| Diethylhexyl Phthalate | general |
| Dihydroxyaluminum Aminoacetate | general |
| Diisopropanolamine | general |
| Diisopropyl Adipate | general |
| Diisopropyl Dilinoleate | general |
| Dimethicone 350 | general |
| Dimethicone Copolyol | general |
| Dimethicone Mdx4-4210 | general |
| Dimethicone Medical Fluid 360 | general |
| Dimethyl Isosorbide | general |
| Dimethylaminoethyl Methacrylate - Butyl Methacrylate - Methyl | general |
| Methacrylate Copolymer | |
| Dimethyldioctadecylammonium Bentonite | general |
| Dimethylsiloxane/Methylvinylsiloxane Copolymer | general |
| Dinoseb Ammonium Salt | general |
| Dipalmitoylphosphatidylglycerol (DL-) | general |
| Dipropylene Glycol | general |
| Disodium Cocoamphodiacetate | general |
| Disodium Laureth Sulfosuccinate | general |
| Disodium Lauryl Sulfosuccinate | general |
| Disodium Sulfosalicylate | general |
| Disofenin | general |
| Divinylbenzene Styrene Copolymer | general |
| Dmdm Hydantoin | general |
| Docosanol | general |
| Docusate Sodium | general |
| Duro-Tak 280-2516 | general |
| Duro-Tak 387-2516 | general |
| Duro-Tak 80-1196 | general |
| Duro-Tak 87-2070 | general |
| Duro-Tak 87-2194 | general |
| Duro-Tak 87-2287 | general |
| Duro-Tak 87-2296 | general |
| Duro-Tak 87-2888 | general |
| Duro-Tak 87-2979 | general |
| Edetate Calcium Disodium | general |
| Edetate Disodium | general |
| Edetate Disodium Anhydrous | general |
| Edetate Sodium | general |
| Edetic Acid | general |
| Egg | general |
| Egg Phospholipid | general |
| Entsufon | general |
| Entsufon Sodium | general |
| Epilactose | general |
| Epitetracycline Hydrochloride | general |
| Essence Bouquet 9200 | general |
| Ethanolamine Hydrochloride | general |
| Ethoxylated | general |
| Ethyl Ester Terminated | general |
| Ethyl Oleate | general |
| Ethylene Glycol | general |
| Ethylene Vinyl Acetate Copolymer | general |
| Ethylenediamine | general |
| Ethylenediamine Dihydrochloride | general |
| Ethylenediaminetetracetic acid (EDTA) | general |
| Ethylene-Propylene Copolymer | general |
| Ethylene-Vinyl Acetate Copolymer (28% Vinyl Acetate) | general |
| Ethylene-Vinyl Acetate Copolymer (9% Vinylacetate) | general |
| Ethylhexyl Hydroxystearate | general |
| Ethylparaben | general |
| Eucalyptol | general |
| Exametazime | general |
| F&C Red No. 40 | general |
| Fat (Edible) | general |
| Fat (Hard) | general |
| Fatty Acid | general |
| Fatty Acid Ester | general |
| Fatty Acid Pentaerythriol Ester | general |
| Fatty Alcohol | general |
| Fatty Alcohol Citrate | general |
| FD&C Blue No. 1 (brilliant blue FCF) | general |
| FD&C Green No. 3 (fast green FCF) | general |
| FD&C Red No. 4 | general |
| FD&C Yellow No. 10 (Delisted) | general |
| FD&C Yellow No. 5 (tartrazine) | general |
| FD&C Yellow No. 6 (sunset yellow) | general |
| Ferric Chloride | general |
| Ferric Oxide | general |
| Flavor 89-186 | general |
| Flavor 89-259 | general |
| Flavor Df-119 | general |
| Flavor Df-1530 | general |
| Flavor Enhancer | general |
| Flavor Fig 827118 | general |
| Flavor Raspberry Pfc-8407 | general |
| Flavor Rhodia Pharmaceutical No. Rf 451 | general |
| Fluorochlorohydrocarbon | general |
| Formaldehyde | general |
| Formaldehyde Solution | general |
| Fractionated Coconut Oil | general |
| Fragrance 3949-5 | general |
| Fragrance 520a | general |
| Fragrance 6.007 | general |
| Fragrance 91-122 | general |
| Fragrance 9128-Y | general |
| Fragrance 93498g | general |
| Fragrance Balsam Pine No. 5124 | general |
| Fragrance Bouquet 10328 | general |
| Fragrance Chemoderm 6401-B | general |
| Fragrance Chemoderm 6411 | general |
| Fragrance Cream No. 73457 | general |
| Fragrance Cs-28197 | general |
| Fragrance Felton 066m | general |
| Fragrance Firmenich 47373 | general |
| Fragrance Givaudan Ess 9090/lc | general |
| Fragrance H-6540 | general |
| Fragrance Herbal 10396 | general |
| Fragrance Nj-1085 | general |
| Fragrance P O Fl-147 | general |
| Fragrance Pa 52805 | general |
| Fragrance Pera Derm D | general |
| Fragrance Rbd-9819 | general |
| Fragrance Shaw Mudge U-7776 | general |
| Fragrance Tf 044078 | general |
| Fragrance Ungerer Honeysuckle K 2771 | general |
| Fragrance Ungerer N5195 | general |
| Fructose | general |
| Gadolinium Oxide | general |
| Galactose | general |
| Gamma Cyclodextrin | general |
| Gelatin (Crosslinked) | general |
| Gelfoam Sponge | general |
| Gellan Gum (Low Acyl) | general |
| Gelva 737 | general |
| Gentisic Acid | general |
| Gentisic Acid Ethanolamide | general |
| Glacial acetic acid | general |
| Gluceptate Sodium | general |
| Gluceptate Sodium Dihydrate | general |
| Gluconolactone | general |
| Glucuronic Acid | general |
| Glutamic Acid (DL-) | general |
| Glutathione | general |
| Glycerol Ester Of Hydrogenated Rosin | general |
| Glyceryl Citrate | general |
| Glyceryl Isostearate | general |
| Glyceryl Laurate | general |
| Glyceryl Monostearate | general |
| Glyceryl Oleate | general |
| Glyceryl Oleate/Propylene Glycol | general |
| Glyceryl Palmitate | general |
| Glyceryl Ricinoleate | general |
| Glyceryl Stearate | general |
| Glyceryl Stearate - Laureth-23 | general |
| Glyceryl Stearate/Peg Stearate | general |
| Glyceryl Stearate/Peg-100 Stearate | general |
| Glyceryl Stearate/Peg-40 Stearate | general |
| Glyceryl Stearate-Stearamidoethyl Diethylamine | general |
| Glyceryl Trioleate | general |
| Glycine | general |
| Glycine Hydrochloride | general |
| Glycol Distearate | general |
| Glycol Stearate | general |
| Guanidine Hydrochloride | general |
| Guar Gum | general |
| Hair Conditioner (18n195-1m) | general |
| Heptane | general |
| Hetastarch | general |
| Hexylene Glycol | general |
| High Density Polyethylene | general |
| Histidine | general |
| Human Albumin Microspheres | general |
| Hyaluronate Sodium | general |
| Hydrocarbon | general |
| Hydrocarbon Gel | general |
| Hydrochloric Acid | general |
| Hydrocortisone | general |
| Hydrogel Polymer | general |
| Hydrogen Peroxide | general |
| Hydrogenated Castor Oil | general |
| Hydrogenated coconut oil | general |
| Hydrogenated Coconut Oil Glyceride | general |
| Hydrogenated palm kernel oil | general |
| Hydrogenated Palm Kernel Oil glyceride | general |
| Hydrogenated Palm Oil | general |
| Hydrogenated Palm/Palm Kernel Oil Peg-6 Ester | general |
| Hydrogenated Polybutene 635-690 | general |
| Hydrogenated Soy | general |
| Hydrogenated soy phosphotidylcholine | general |
| Hydroxide Ion | general |
| Hydroxyethylpiperazine Ethane Sulfonic Acid | general |
| Hydroxymethyl Cellulose | general |
| Hydroxyoctacosanyl Hydroxystearate | general |
| Hydroxypropyl Methylcellulose 2906 | general |
| Hydroxypropyl-B-cyclodextrin | general |
| Hypromellose | general |
| Hypromellose 2208 (15000 Mpa · S) | general |
| Hypromellose 2910 (15000 Mpa · S) | general |
| Imidurea | general |
| Iodine | general |
| Iodoxamic Acid | general |
| Iofetamine Hydrochloride | general |
| Irish Moss Extract | general |
| Isobutane | general |
| Isoceteth-20 | general |
| Isoleucine | general |
| Isooctyl Acrylate | general |
| Isopropyl Alcohol | general |
| Isopropyl Isostearate | general |
| Isopropyl Myristate | general |
| Isopropyl Palmitate | general |
| Isopropyl Stearate | general |
| Isostearic Acid | general |
| Isostearyl Alcohol | general |
| Isotonic Sodium Chloride Solution | general |
| Jelene | general |
| Kaolin | general |
| Kathon Cg | general |
| Kathon Cg II | general |
| Lactate | general |
| Lactic Acid | general |
| Lactic Acid (DL-) | general |
| Lactobionic Acid | general |
| Lactose | general |
| Lactose hydrous | general |
| Lactose Monohydrate | general |
| Laneth | general |
| Lanolin | general |
| Lanolin (ethoxylated) | general |
| Lanolin (hydrogenated) | general |
| Lanolin Alcohol | general |
| Lanolin Anhydrous | general |
| Lanolin Cholesterol | general |
| Lanolin Nonionic Derivatives | general |
| Lauralkonium Chloride | general |
| Lauramine Oxide | general |
| Laurdimonium Hydrolyzed Animal Collagen | general |
| Laureth Sulfate | general |
| Laureth-2 | general |
| Laureth-23 | general |
| Laureth-4 | general |
| Lauric Diethanolamide | general |
| Lauric Myristic Diethanolamide | general |
| Lauroyl Sarcosine | general |
| Lauryl Lactate | general |
| Lauryl Sulfate | general |
| Lavandula Angustifolia Flowering Top | general |
| Lecithin | general |
| Lecithin (hydrogenated) | general |
| Lecithin Unbleached | general |
| Lemon Oil | general |
| Leucine | general |
| Levulinic Acid | general |
| Lidofenin | general |
| Light Mineral Oil | general |
| Light Mineral Oil (85 Ssu) | general |
| Limonene (+/−) | general |
| Lipocol Sc-15 | general |
| Lysine | general |
| Lysine Acetate | general |
| Lysine Monohydrate | general |
| Magnesium Aluminum Silicate Hydrate | general |
| Magnesium Chloride | general |
| Magnesium Nitrate | general |
| Magnesium Stearate | general |
| Maleic Acid | general |
| Maltitol | general |
| Maltodextrin | general |
| Mannitol | general |
| Mannose | general |
| Maprofix | general |
| Mebrofenin | general |
| Medical Adhesive Modified S-15 | general |
| Medical Antiform A-F Emulsion | general |
| Medium Chain | general |
| Medronate Disodium | general |
| Medronic Acid | general |
| Meglumine | general |
| Melezitose | general |
| Menthol | general |
| Metacresol | general |
| Metaphosphoric Acid | general |
| Methanesulfonic Acid | general |
| Methionine | general |
| Methyl Alcohol | general |
| Methyl Gluceth-10 | general |
| Methyl Gluceth-20 | general |
| Methyl Gluceth-20 Sesquistearate | general |
| Methyl Glucose Sesquistearate | general |
| Methyl Laurate | general |
| Methyl Pyrrolidone | general |
| Methyl Salicylate | general |
| Methyl Stearate | general |
| Methylboronic Acid | general |
| Methylcellulose (4000 Mpa · S) | general |
| Methylchloroisothiazolinone | general |
| Methylene Blue | general |
| Methylisothiazolinone | general |
| Methylparaben | general |
| Microcrystalline | general |
| Microcrystalline Wax | general |
| Mineral Oil | general |
| Monostearyl Citrate | general |
| Monothioglycerol | general |
| Multisterol Extract | general |
| Myristyl Alcohol | general |
| Myristyl Lactate | general |
| Myristyl-.Gamma.-Picolinium Chloride | general |
| N-(Carbamoyl-Methoxy Peg-40)-1,2-Distearoyl-Cephalin Sodium | general |
| N,N-Dimethylacetamide | general |
| Niacinamide | general |
| Nioxime | general |
| Nitric Acid | general |
| Nitrogen | general |
| Nonoxynol Iodine | general |
| Nonoxynol-15 | general |
| Nonoxynol-9 | general |
| Norflurane | general |
| Oatmeal | general |
| Octadecene-1/Maleic Acid Copolymer | general |
| Octanoic Acid | general |
| Octisalate | general |
| Octoxynol-1 | general |
| Octoxynol-40 | general |
| Octoxynol-9 | general |
| Octyldodecanol | general |
| Octylphenol Polymethylene | general |
| Oleth-10/Oleth-5 | general |
| Oleth-2 | general |
| Oleth-20 | general |
| Oleyl Alcohol | general |
| Oleyl Oleate | general |
| Oxidronate Disodium | general |
| Oxyquinoline | general |
| Palm Kernel Oil | general |
| Palm Kernel Oil Glyceride | general |
| Palmitamine Oxide | general |
| Parabens | general |
| Paraffin | general |
| Parfum Creme 45/3 | general |
| Peanut Oil (Refined) | general |
| Pectin | general |
| Peg 6-32 Stearate/Glycol Stearate | general |
| Peg Vegetable Oil | general |
| Peg-100 Stearate | general |
| Peg-12 Glyceryl Laurate | general |
| Peg-120 Glyceryl Stearate | general |
| Peg-120 Methyl Glucose Dioleate | general |
| Peg-15 Cocamine | general |
| Peg-150 Distearate | general |
| Peg-2 Stearate | general |
| Peg-20 Sorbitan Isostearate | general |
| Peg-22 Methyl Ether/Dodecyl Glycol Copolymer | general |
| Peg-25 Propylene Glycol Stearate | general |
| Peg-4 Dilaurate | general |
| Peg-4 Laurate | general |
| Peg-40 Castor Oil | general |
| Peg-40 Sorbitan Diisostearate | general |
| Peg-45/Dodecyl Glycol Copolymer | general |
| Peg-5 Oleate | general |
| Peg-50 Stearate | general |
| Peg-54 Hydrogenated Castor Oil | general |
| Peg-6 Isostearate | general |
| Peg-60 Castor Oil | general |
| Peg-60 Hydrogenated Castor Oil | general |
| Peg-7 Methyl Ether | general |
| Peg-75 Lanolin | general |
| Peg-8 Laurate | general |
| Peg-8 Stearate | general |
| Pegoxol 7 Stearate | general |
| Pentadecalactone | general |
| Pentaerythritol Cocoate | general |
| Pentasodium Pentetate | general |
| Pentetate Calcium Trisodium | general |
| Pentetic Acid | general |
| Perflutren | general |
| Perfume 25677 | general |
| Perfume Bouquet | general |
| Perfume E-1991 | general |
| Perfume Gd 5604 | general |
| Perfume Tana 90/42 Scba | general |
| Perfume W-1952-1 | general |
| Petrolatum | general |
| Petroleum Distillate | general |
| Phenol | general |
| Phenol (Liquefied) | general |
| Phenonip | general |
| Phenoxyethanol | general |
| Phenylalanine | general |
| Phenylethyl Alcohol | general |
| Phenylmercuric Acetate | general |
| Phenylmercuric Nitrate | general |
| Phosphate buffer | general |
| Phosphate buffered saline | general |
| Phosphate salts | general |
| Phosphatidyl Glycerol | general |
| Phospholipid | general |
| Phospholipid (Egg) | general |
| Phospholipon 90g | general |
| Phosphoric Acid | general |
| Pine Needle Oil (Finns Sylvestris) | general |
| Piperazine Hexahydrate | general |
| Plastibase-50w | general |
| Polacrilin | general |
| Polidronium Chloride | general |
| Poloxamer 124 | general |
| Poloxamer 181 | general |
| Poloxamer 182 | general |
| Poloxamer 188 | general |
| Poloxamer 237 | general |
| Poloxamer 407 | general |
| Poly(Bis(P-Carboxyphenoxy)Propane Anhydride): Sebacic Acid | general |
| Poly(Dimethylsiloxane/Methylvinylsiloxane/Methylhydrogensiloxane) | general |
| Dimethylvinyl Or Dimethylhydroxy Or Trimethyl Endblocked | |
| Poly(D1-Lactic-Co-Glycolic Acid) | general |
| Polybutene (1400 Mw) | general |
| Polycarbophil | general |
| Polyester Polyamine Copolymer | general |
| Polyester Rayon | general |
| Polyethylene Glycol 1000 | general |
| Polyethylene Glycol 1450 | general |
| Polyethylene Glycol 1500 | general |
| Polyethylene Glycol 1540 | general |
| Polyethylene Glycol 200 | general |
| Polyethylene Glycol 300 | general |
| Polyethylene Glycol 300-1600 | general |
| Polyethylene Glycol 3350 | general |
| Polyethylene Glycol 400 (PEG 400) | general |
| Polyethylene Glycol 4000 (PEG 4000, PEG 4 kDa) | general |
| Polyethylene Glycol 540 | general |
| Polyethylene Glycol 600 | general |
| Polyethylene Glycol 6000 | general |
| Polyethylene Glycol 8000 | general |
| Polyethylene Glycol 900 | general |
| Polyethylene High Density Containing Ferric Oxide Black (<1%) | general |
| Polyethylene Low Density Containing Barium Sulfate (20-24%) | general |
| Polyethylene T | general |
| Polyethylene Terephthalate | general |
| Polyglactin | general |
| Polyglyceryl-3 Oleate | general |
| Polyglyceryl-4 Oleate | general |
| Polyhydroxyethyl Methacrylate | general |
| Polyisobutylene | general |
| Polyisobutylene (1100000 Mw) | general |
| Polyisobutylene (35000 Mw) | general |
| Polyisobutylene 178-236 | general |
| Polyisobutylene 241-294 | general |
| Polyisobutylene 35-39 | general |
| Polyisobutylene Low Molecular Weight | general |
| Polyisobutylene Medium Molecular Weight | general |
| Polyisobutylene/Polybutene Adhesive | general |
| Polylactide | general |
| Polyol | general |
| Polyoxyethylene | general |
| Polyoxyethylene Alcohol | general |
| Polyoxyethylene Fatty Acid Ester | general |
| Polyoxyethylene Propylene | general |
| Polyoxyl 20 Cetostearyl Ether | general |
| Polyoxyl 32 Palmitostearate | general |
| Polyoxyl 35 Castor Oil | general |
| Polyoxyl 40 Hydrogenated Castor Oil | general |
| Polyoxyl 40 Stearate | general |
| Polyoxyl 400 Stearate | general |
| Polyoxyl 6 | general |
| Polyoxyl Distearate | general |
| Polyoxyl Glyceryl Stearate | general |
| Polyoxyl Lanolin | general |
| Polyoxyl Palmitate | general |
| Polyoxyl Stearate | general |
| Polyoxypropylene 1800 | general |
| Polypropylene | general |
| Polypropylene Glycol | general |
| Polyquaternium-10 | general |
| Polyquaternium-7 (70/30) Acrylamide/Dadmac | general |
| Polysiloxane | general |
| Polysorbate 40 | general |
| Polysorbate 60 | general |
| Polysorbate 65 | general |
| Polyurethane | general |
| Polyvinyl Acetate | general |
| Polyvinyl Chloride | general |
| Polyvinyl Chloride-Polyvinyl Acetate Copolymer | general |
| Polyvinylpyridine | general |
| Poppy Seed Oil | general |
| Potash | general |
| Potassium Acetate | general |
| Potassium Alum | general |
| Potassium Bicarbonate | general |
| Potassium Bisulfite | general |
| Potassium Chloride | general |
| Potassium Citrate | general |
| Potassium Hydroxide | general |
| Potassium Metabisulfite | general |
| Potassium Phosphate (Dibasic) | general |
| Potassium Phosphate (Monobasic) | general |
| Potassium Soap | general |
| Povidone | general |
| Povidone Acrylate Copolymer | general |
| Povidone Hydrogel | general |
| Povidone K17 | general |
| Povidone K25 | general |
| Povidone K29/32 | general |
| Povidone K30 | general |
| Povidone K90 | general |
| Povidone K90f | general |
| Povidone/Eicosene Copolymer | general |
| Ppg-12/Smdi Copolymer | general |
| Ppg-15 Stearyl Ether | general |
| Ppg-20 Methyl Glucose Ether Distearate | general |
| Ppg-26 Oleate | general |
| Pregelatinized | general |
| Product Wat | general |
| Proline | general |
| Promulgen D | general |
| Promulgen G | general |
| Propane | general |
| Propellant A-46 | general |
| Propyl Gallate | general |
| Propylene Glycol Diacetate | general |
| Propylene Glycol Dicaprylate | general |
| Propylene Glycol Monolaurate | general |
| Propylene Glycol Monopalmilostearate | general |
| Propylene Glycol Palmitostearate | general |
| Propylene Glycol Ricinoleate | general |
| Propylene Glycol/Diazolidinyl Urea/Methylparaben/Propylparben | general |
| Propylparaben | general |
| Protamine Sulfate | general |
| Protein Hydrolysate | general |
| Pvm/Ma Copolymer | general |
| Quaternium-15 | general |
| Quaternium-15 Cis-Form | general |
| Quaternium-52 | general |
| Ra-2397 | general |
| Ra-3011 | general |
| Raffinose | general |
| Saccharin | general |
| Saccharin Sodium | general |
| Saccharin Sodium Anhydrous | general |
| Sd Alcohol 3a | general |
| Sd Alcohol 40 | general |
| Sd Alcohol 40-2 | general |
| Sd Alcohol 40b | general |
| Sepineo P 600 | general |
| Serine | general |
| Shea Butter | general |
| Silastic Brand Medical Grade Tubing | general |
| Silastic Medical Adhesive | general |
| Silica | general |
| Silicon | general |
| Silicon Dioxide | general |
| Silicone | general |
| Silicone Adhesive 4102 | general |
| Silicone Adhesive 4502 | general |
| Silicone Adhesive Bio-Psa Q7-4201 | general |
| Silicone Adhesive Bio-Psa Q7-4301 | general |
| Silicone Emulsion | general |
| Silicone Type A | general |
| Silicone/Polyester Film Strip | general |
| Simethicone | general |
| Simethicone Emulsion | general |
| Sipon Ls 20np | general |
| Soda Ash | general |
| Sodium Acetate | general |
| Sodium Acetate Anhydrous | general |
| Sodium Alkyl Sulfate | general |
| Sodium Ascorbate | general |
| Sodium Benzoate | general |
| Sodium Bicarbonate | general |
| Sodium Bisulfate | general |
| Sodium Borate | general |
| Sodium Borate Decahydrate | general |
| Sodium Carbonate | general |
| Sodium Carbonate Decahydrate | general |
| Sodium Carbonate Monohydrate | general |
| Sodium Cetostearyl Sulfate | general |
| Sodium Chlorate | general |
| Sodium Chloride | general |
| Sodium Chloride Injection | general |
| Sodium Cholesteryl Sulfate | general |
| Sodium Citrate | general |
| Sodium Citrate dihydrate | general |
| Sodium Cocoyl Sarcosinate | general |
| Sodium Desoxycholate | general |
| Sodium Dithionite | general |
| Sodium Dodecylbenzenesulfonate | general |
| Sodium Formaldehyde Sulfoxylate | general |
| Sodium Gluconate | general |
| Sodium Hydroxide | general |
| Sodium Hypochlorite | general |
| Sodium Iodide | general |
| Sodium Lactate | general |
| Sodium Lactate (L-) | general |
| Sodium Laureth-2 Sulfate | general |
| Sodium Laureth-3 Sulfate | general |
| Sodium Laureth-5 Sulfate | general |
| Sodium Lauroyl Sarcosinate | general |
| Sodium Lauryl Sulfate | general |
| Sodium Lauryl Sulfoacetate | general |
| Sodium Metabisulfite | general |
| Sodium Phosphate | general |
| Sodium Phosphate (Dibasic) | general |
| Sodium Phosphate (Dibasic, Anhydrous) | general |
| Sodium Phosphate (Dibasic, Dihydrate) | general |
| Sodium Phosphate (Dibasic, Dodecahydrate) | general |
| Sodium Phosphate (Dibasic, Heptahydrate) | general |
| Sodium Phosphate (Monobasic) | general |
| Sodium Phosphate (Monobasic, Anhydrous) | general |
| Sodium Phosphate (Monobasic, Dihydrate) | general |
| Sodium Phosphate (Monobasic, Monohydrate) | general |
| Sodium Phosphate Dihydrate | general |
| Sodium Polyacrylate (2500000 Mw) | general |
| Sodium Pyrophosphate | general |
| Sodium Pyrrolidone Carboxylate | general |
| Sodium Starch Glycolate | general |
| Sodium Succinate Hexahydrate | general |
| Sodium Sulfate | general |
| Sodium Sulfate Anhydrous | general |
| Sodium Sulfate Decahydrate | general |
| Sodium Sulfite | general |
| Sodium Sulfosuccinated Undecyclenic Monoalkylolamide | general |
| Sodium Tartrate | general |
| Sodium Thioglycolate | general |
| Sodium Thiomalate | general |
| Sodium Thiosulfate | general |
| Sodium Thiosulfate Anhydrous | general |
| Sodium Trimetaphosphate | general |
| Sodium Xylenesulfonate | general |
| Somay 44 | general |
| Sorbic Acid | general |
| Sorbitan | general |
| Sorbitan Isostearate | general |
| Sorbitan Monolaurate | general |
| Sorbitan Monopalmitate | general |
| Sorbitan Monostearate | general |
| Sorbitan Sesquioleate | general |
| Sorbitan Trioleate | general |
| Sorbitan Tristearate | general |
| Sorbitol Solution | general |
| Sorbose | general |
| Soybean | general |
| Soybean Flour | general |
| Spearmint Oil | general |
| Spermaceti | general |
| Squalane | general |
| Stabilized Oxychloro Complex | general |
| Stannous 2-Ethylhexanoate | general |
| Stannous Chloride | general |
| Stannous Chloride Anhydrous | general |
| Stannous Fluoride | general |
| Stannous Tartrate | general |
| Starch | general |
| Starch 1500 | general |
| Stearalkonium Chloride | general |
| Stearalkonium Hectorite/Propylene Carbonate | general |
| Stearamidoethyl Diethylamine | general |
| Steareth-10 | general |
| Steareth-100 | general |
| Steareth-2 | general |
| Steareth-20 | general |
| Steareth-21 | general |
| Steareth-40 | general |
| Stearic Acid | general |
| Stearic Diethanolamide | general |
| Stearoxytrimethylsilane | general |
| Steartrimonium Hydrolyzed Animal Collagen | general |
| Stearyl Alcohol | general |
| Styrene/Isoprene/Styrene Block Copolymer | general |
| Succimer | general |
| Succinic Acid | general |
| Sucralose | general |
| Sucrose | general |
| Sucrose Distearate | general |
| Sugar | general |
| Sucrose Polyester | general |
| Sulfacetamide Sodium | general |
| Sulfobutylether.Beta.-Cyclodextrin | general |
| Sulfur Dioxide | general |
| Sulfuric Acid | general |
| Sulfurous Acid | general |
| Surfactol Qs | general |
| Tagatose (D-) | general |
| Talc | general |
| Tall Oil | general |
| Tallow Glycerides | general |
| Tartaric Acid (DL-) | general |
| Tenox | general |
| Tenox-2 | general |
| Tert-Butyl Alcohol | general |
| Tert-Butyl Hydroperoxide | general |
| Tert-Butylhydroquinone | general |
| Tetrakis(2-Methoxyisobutylisocyanide)Copper(I) Tetrafluoroborate | general |
| Tetrapropyl Orthosilicate | general |
| Tetrofosmin | general |
| Theophylline | general |
| Thimerosal | general |
| Threonine | general |
| Thymol | general |
| Tin | general |
| Titanium Dioxide | general |
| Tocopherol | general |
| Trehalose | general |
| Tocophersolan | general |
| Tricaprylin | general |
| Trichloromonofluoromethane | general |
| Trideceth-10 | general |
| Triethanolamine Lauryl Sulfate | general |
| Trifluoroacetic Acid | general |
| Triglycerides | general |
| Trihalose | general |
| Trihydroxystearin | general |
| Trilaneth-4 Phosphide | general |
| Trilaureth-4 Phosphate | general |
| Trisodium Citrate Dihydrate | general |
| Trisodium Hedta | general |
| Triton 720 | general |
| Triton X-200 | general |
| Trolamine | general |
| Tromantadine | general |
| Tromethamine | general |
| Tryptophan | general |
| Tyloxapol | general |
| Tyrosine | general |
| Undecylenic Acid | general |
| Union 76 Amsco-Res 6038 | general |
| Urea | general |
| Valine | general |
| Vegetable Oil | general |
| Vegetable Oil Glyceride | general |
| Versetamide | general |
| Viscarin | general |
| Viscose/Cotton | general |
| Vitamin E | general |
| Water | general |
| Wax | general |
| Wecobee F | general |
| White | general |
| White Ceresin Wax | general |
| White Soft | general |
| White Wax | general |
| Zinc | general |
| Zinc Acetate | general |
| Zinc Carbonate | general |
| Zinc Chloride | general |
| Zinc Oxide | general |
| DSPC | lipid nanoparticle |
| lipid nanoparticle | lipid nanoparticle |
| PEG-DMG 2000 (1,2-dimyristoyl-sn-glycero-3-phophoethanolamine- | lipid nanoparticle |
| N-[methoxy(polyethylene glycol)-2000) | |
| 1,2-dilauroyl-sn-glycero-3-phosphocholine (DLPC) | lipids |
| 1,2-dimyristoyl-sn-glycero-3-phosphocholine | lipids |
| (DMPC)phosphatidylinositol | |
| 1,2-dioleoyl-sn-glycero-3-phophoethanolamine (DOPE) | lipids |
| 1,2-dioleoyl-sn-glycero-3-phosphocholine (DOPC) | lipids |
| diglyceride | lipids |
| dilinoleoylphosphatidylcholine | lipids |
| dioleoylphosphatidylcholine | lipids |
| dipalmitoylphosphatidylcholine | lipids |
| distearoylphosphatidylcholine | lipids |
| fats | lipids |
| lysolipids | lipids |
| lysophosphatidylethanolamine | lipids |
| lysophospholipid | lipids |
| monoglyceride | lipids |
| mono-myristoyl-phosphatidylethanolamine (MMPE) | lipids |
| mono-oleoyl-phosphatidic acid (MOPA) | lipids |
| mono-oleoyl-phosphatidylethanolamine (MOPE) | lipids |
| mono-oleoyl-phosphatidylglycerol (MOPG) | lipids |
| mono-oleoyl-phosphatidylserine (MOPS) | lipids |
| palmitoyloleoyl | lipids |
| palmitoyloleoyl phosphatidylcholine | lipids |
| palmitoyl-oleoyl-phosphatidylethanolamine (POPE) | lipids |
| phosphatidic acid | lipids |
| phosphatidylcholines | lipids |
| phosphatidylethanolamine | lipids |
| phosphatidylserine | lipids |
| phosphotidylglycerol | lipids |
| sterol | lipids |
| 1,2-dilinoleyloxy-3-dimethylaminopropane (DLin-DMA) | liposomes |
| 1,2-dioleyloxy-N,N-dimethylaminopropane (DODMA) liposomes | liposomes |
| 2,2-dilinoleyl-4-(2-dimethylaminoethyl)-[1,3]-dioxolane (DLin-KC2- | liposomes |
| DMA) | |
| DiLa2 liposomes from Marina Biotech (Bothell, WA) | liposomes |
| hyaluronan-coated liposomes | liposomes |
| liposome | liposomes |
| MC3 | liposomes |
| neutral DOPC (1,2-dioleoyl-sn-glycero-3-phosphocholine) based | liposomes |
| liposome | |
| SMARTICLES ® (Marina Biotech, Bothell, WA) | liposomes |
| stabilized nucleic acid lipid particle (SNALP) | liposomes |
| stabilized plasmid-lipid particles (SPLP) | liposomes |
| alkali salt | lubricant |
| alkaline earth salt | lubricant |
| aqueous solution | lubricant |
| calcium stearate | lubricant |
| fumed silica | lubricant |
| high molecular weight polyalkylene glycol | lubricant |
| high molecular weight polyethylene glycol | lubricant |
| hyaluronic acid | lubricant |
| hydrogenated vegetable oil | lubricant |
| hydrous magnesium silicate | lubricant |
| lipids | lubricant |
| lubricants | lubricant |
| lubricin | lubricant |
| micelle | lubricant |
| microsphere | lubricant |
| monoester of propylene glycol | lubricant |
| oils | lubricant |
| polymer | lubricant |
| saturated fatty acid containing about 16-20 carbon atoms | lubricant |
| saturated fatty acid containing about 8-22 carbon atoms | lubricant |
| solvents | lubricant |
| stearate salts | lubricant |
| transition metal salt | lubricant |
| vegetable oil derivative | lubricant |
| acrylic acid | nanoparticles |
| acrylic polymer | nanoparticles |
| amino alkyl methacrylate copolymer | nanoparticles |
| anhydride-modified material | nanoparticles |
| anhydride-modified phytoglycogen beta-dextrin | nanoparticles |
| carbon nanoparticles | nanoparticles |
| ceramic silicon carbide nanoparticle | nanoparticles |
| cerium oxide nanoparticle | nanoparticles |
| curcumin nanoparticle | nanoparticles |
| cyanoethyl methacrylate | nanoparticles |
| DLin-KC2-DMA | nanoparticles |
| DLin-MC3-DMA | nanoparticles |
| ethoxyethyl methacrylate | nanoparticles |
| glycogen-type material | nanoparticles |
| gold nanoparticle | nanoparticles |
| iron nanoparticles | nanoparticles |
| iron oxide nanoparticle | nanoparticles |
| magnetic nanoparticle | nanoparticles |
| methacrylic acid | nanoparticles |
| methacrylic acid copolymer | nanoparticles |
| methyl methacrylate copolymer | nanoparticles |
| nanodiamond | nanoparticles |
| nickel nanoparticle | nanoparticles |
| phytoglycogen beta-dextrin | nanoparticles |
| phytoglycogen octenyl succinate | nanoparticles |
| platinum nanoparticles | nanoparticles |
| poly(4-hydroxy-L-proline ester) | nanoparticles |
| poly(acrylic acid) | nanoparticles |
| poly(ethylene imine) | nanoparticles |
| poly(L-lactide-co-L-lysine) | nanoparticles |
| poly(methacrylic acid) | nanoparticles |
| poly(orthoesters) | nanoparticles |
| poly(serine ester) | nanoparticles |
| polyacetal | nanoparticles |
| polyacrylate | nanoparticles |
| polycyanoacrylate | nanoparticles |
| polyester | nanoparticles |
| polyether | nanoparticles |
| polyethylene | nanoparticles |
| polyhydroxyacid | nanoparticles |
| polylysine | nanoparticles |
| polymer coated iron oxide nanoparticle | nanoparticles |
| polymeric mycelle | nanoparticles |
| polymethacrylate | nanoparticles |
| polyphosphazene | nanoparticles |
| polypropylfumerate | nanoparticles |
| polyureas | nanoparticles |
| protein filled nanoparticle | nanoparticles |
| silica nanoparticle | nanoparticles |
| silicon dioxide crystalline nanoparticle | nanoparticles |
| silver nanoparticles | nanoparticles |
| silver oxide nanoparticle | nanoparticles |
| titanium dioxide nanoparticle | nanoparticles |
| natural polymers | natural polymers |
| natural rubbers | natural polymers |
| ceramic | other |
| cobalt-chromium-molydenum composite | other |
| duck's feet collagen | other |
| ionic liquids | other |
| magnesium oxide | other |
| melanin | other |
| metal scaffold | other |
| nano-hydroxyapatite | other |
| poly(α-ester) | other |
| SBA15 | other |
| alginate | polymers |
| alkyl cellulose | polymers |
| amber | polymers |
| bacterial cellulose | polymers |
| bioplastic | polymers |
| bioresorbable polymer matrix | polymers |
| carbohydrate polymers | polymers |
| cellulose acetate | polymers |
| cellulose ester | polymers |
| cellulose ether | polymers |
| chitin | polymers |
| chitosan | polymers |
| copolymers of acrylic and methacrylic acid esters | polymers |
| derivatized cellulose | polymers |
| elastin | polymers |
| ethylene vinyl acetate polymer (EVA) | polymers |
| EUDRAGIT ® RL | polymers |
| EUDRAGIT ® RS | polymers |
| fibrin | polymers |
| genetically modified bioplastics | polymers |
| glycogen | polymers |
| high-density polyethylene (HDPE) | polymers |
| hydroxypropyl methylcellulose (HPMC) | polymers |
| hydroxyalkyd celluloses | polymers |
| hydroxypropyl ethylcellulose (HEC) | polymers |
| hydroxypropyl methacrylate (HPMA) | polymers |
| hydroxypropylcellulose | polymers |
| keratins | polymers |
| lignin | polymers |
| lipid-derived polymer | polymers |
| low-density polyethylene (LDPE) | polymers |
| methacrylates | polymers |
| natural rubber | polymers |
| neoprene | polymers |
| nitro cellulose | polymers |
| nucleic acid | polymers |
| nylon | polymers |
| nylon 6 | polymers |
| nylon 6.6 | polymers |
| nylone | polymers |
| phenol formaldehyde resin | polymers |
| poloxamer | polymers |
| poly(butyl(meth)acrylate) | polymers |
| poly(butyric acid) | polymers |
| poly(caprolactone) (PCL) | polymers |
| poly(D,L-lactide) (PDLA) | polymers |
| poly(D,L-lactide-co-caprolactone) | polymers |
| poly(D,L-lactide-co-caprolactone-co-glycolide) | polymers |
| poly(D,L-lactide-co-PPO-co-D,L-lactide) | polymers |
| poly(ester amides) | polymers |
| poly(ester ethers) | polymers |
| poly(ethyl(meth)acrylate) | polymers |
| poly(ethylene terephthalate) | polymers |
| poly(glycolic acid) (PGA) | polymers |
| poly(hexyl(meth)acrylate) | polymers |
| poly(hydroxy acids) | polymers |
| poly(isobutyl acrylate) | polymers |
| poly(isobutyl(meth)acrylate) | polymers |
| poly(isodecyl(meth)actylate) | polymers |
| poly(isopropyl acrylate) | polymers |
| poly(lactic acid) (PLA) | polymers |
| poly(lactic acid-co-glycolic acid) (PLGA) | polymers |
| poly(lactide-co-caprolactone) | polymers |
| poly(lactide-co-glycolide) | polymers |
| poly(lauryl(meth)acrylate) | polymers |
| poly(L-lactic acid) (PLLA) | polymers |
| poly(L-lactic acid-co-glycolic acid) (PLLGA) | polymers |
| poly(L-lactide) (PLLA) | polymers |
| poly(methyl acrylate) | polymers |
| poly(methyl(meth)acrylate) (PMMA) | polymers |
| poly(octadecyl acrylate) | polymers |
| poly(ortho)esters | polymers |
| poly(phenyl(meth)acrylate) | polymers |
| β-keratin | polymers |
| alkylparaben | preservative |
| amino acids | preservative |
| Antioxidant | preservative |
| BHA | preservative |
| BHT | preservative |
| calcium propionate | preservative |
| disodium EDTA | preservative |
| glutaraldehyde | preservative |
| magnesium chloride hexahydrate | preservative |
| m-cresol | preservative |
| methyl paraben | preservative |
| o-cresol | preservative |
| p-cresol | preservative |
| phenylmercuric nitrite | preservative |
| potassium hydrogen sulfite | preservative |
| potassium sorbate | preservative |
| preservative | preservative |
| propyl paraben | preservative |
| selenium | preservative |
| sodium dehydroacetate | preservative |
| sodium nitrate | preservative |
| sodium nitrite | preservative |
| sulfites | preservative |
| vitamin A | preservative |
| vitamin C | preservative |
| acesulfame potassium | sweetener |
| advantame | sweetener |
| artificial sweetener | sweetener |
| aspartame | sweetener |
| brazzein | sweetener |
| curculin | sweetener |
| cyclamates | sweetener |
| erythritol | sweetener |
| glucose | sweetener |
| glycyrrhizin | sweetener |
| hydrogenated starch hydrolysate | sweetener |
| inulin | sweetener |
| ismalt | sweetener |
| isomaltooligosaccharide | sweetener |
| isomaltulose | sweetener |
| lactitol | sweetener |
| lead acetate | sweetener |
| mabinlin | sweetener |
| miraculin | sweetener |
| mogroside | sweetener |
| monantin | sweetener |
| neotame | sweetener |
| osladin | sweetener |
| pentadin | sweetener |
| polydextrose | sweetener |
| psicose | sweetener |
| stevia | sweetener |
| sweetener | sweetener |
| tagatose | sweetener |
| thaumatin | sweetener |
| xylitol | sweetener |
| xylose | sweetener |
| elastomer | synthetic polymer |
| synthetic fiber | synthetic polymer |
| synthetic polymer | synthetic polymer |
| thermoplastic | synthetic polymer |
| thermoset | synthetic polymer |
| Non-polymeric diol | Demulcent |
| Non-polymeric glycol | Demulcent |
| Cellulose derivative | Demulcent |
| Dextran 70 | Demulcent |
| Cationic cellulose derivative | Demulcent |

In one embodiment, the excipient is sorbitol.

In one embodiment, the excipient is mannitol.

### Polymers

In some embodiments, excipients may include polymers. As used herein, the term “polymer” refers to any substance formed through linkages between similar modules or units. Individual units are referred to herein as “monomers.” Common polymers found in nature include, but are not limited to, carbon chains (e.g., lipids), polysaccharides, nucleic acids, and proteins. In some embodiments, polymers may be synthetic (e.g., thermoplastics, thermosets, elastomers, and synthetic fibers), natural (e.g., chitosan, cellulose, polysaccharides, glycogen, chitin, polypeptides, β-keratins, nucleic acids, natural rubber, etc.), or a combination thereof. In some embodiments, polymers may be irradiated. Non limiting examples of polymers include ethylcellulose and co-polymers of acrylic and methacrylic acid esters (EUDRAGIT® RS or RL), alginates, sodium carboxymethylcellulose, carboxypolymethylene, hydroxpropyl methylcellulose, hydroxypropyl cellulose, collagen, hydroxypropyl ethylcellulose, hydroxyethylcellulose, methylcellulose, xanthum gum, polyethylene oxide, polyethylene glycol, polysiloxane, polyphosphazene, low-density polyethylene (LDPE), high-density polyethylene (HDPE), polyvinyl chloride, polystyrene, nylon, nylon 6, nylon 6.6, polytetrafluoroethylene, thermoplastic polyurethanes, polycaprolactone, polyamide, polycarbonate, chitosan, cellulose, polysaccharides, glycogen, starch, chitin, polypeptides, keratins, β-keratins, nucleic acids, natural rubber, hyaluronan, polylactic acid, methacrylates, polyisoprene, shellac, amber, wool, synthetic rubber, silk, phenol formaldehyde resin, neoprene, nylon, polyacrylonitrile, silicone, polyvinyl butyral, polyhydroxybutyrate (also known as polyhydroxyalkanoate), polyhydroxyurethanes, bioplastics, genetically modified bioplastics, lipid-derived polymers, lignin, carbohydrate polymers, ultra-high-molecular-weight-polyethylene (UHMWPE), gelatin, dextrans, and polyamino acids.

Specific non-limiting examples of specific polymers include, but are not limited to poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone. In some embodiments, polymer excipients may include any of those presented in Table 1, above.

### Particles

In some embodiments, excipients may include particles. Such particles may be of any size and shape, depending on the nature of associated SBPs. In some embodiments, excipient particles are nanoparticles. Non-limiting examples of nanoparticles include gold nanoparticles, silver nanoparticles, silver oxide nanoparticles, iron nanoparticles, iron oxide nanoparticles, platinum nanoparticles, silica nanoparticles, titanium dioxide nanoparticles, magnetic nanoparticles, cerium oxide nanoparticles, protein filled nanoparticles, carbon nanoparticles, nanodiamonds, curcumin nanoparticles, polymeric mycelles, polymer coated iron oxide nanoparticles, ceramic silicon carbide nanoparticles, nickel nanoparticles, and silicon dioxide crystalline nanoparticles.

In some embodiments, nanoparticles may include carbohydrate nanoparticles. Carbohydrate nanoparticles may include carbohydrate carriers. As a non-limiting example, carbohydrate carriers may include, but are not limited to, anhydride-modified or glycogen-type materials, phytoglycogen octenyl succinate, phytoglycogen beta-dextrin, or anhydride-modified phytoglycogen beta-dextrin. (See e.g., International Publication Number WO2012109121, the contents of which are herein incorporated by reference in their entirety).

In some embodiments, excipient nanoparticles may include lipid nanoparticles. Lipid nanoparticle excipients may be carriers in some embodiments. In some embodiments, lipid nanoparticles may be formulated with cationic lipids. In some embodiments, cationic lipids may be biodegradable cationic lipids. Such cationic lipids may be used to form rapidly eliminated lipid nanoparticles. Cationic lipids may include, but are not limited, DLinDMA, DLin-KC2-DMA, and DLin-MC3-DMA. Biodegradable lipid nanoparticles may be used to avoid toxicity associated with accumulation of more stable lipid nanoparticles in plasma and tissues over time.

In some embodiments, nanoparticles include polymeric matrices. As used herein, the term “polymeric matrix” refers to a network of polymer fibers that are bound together to form a material. The polymer fibers may be uniform or may include different lengths or monomer subunits. In some embodiments, polymer matrices may include one or more of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), or combinations thereof.

In some embodiments, polymers include diblock copolymers. As used herein, the term “diblock copolymer” refers to polymers with two different monomer chains grafted to form a single chain. Diblock polymers may be designed to aggregate in different ways, including aggregation as a particle. In some embodiments, diblock copolymers include polyethylene glycol (PEG) in combination with polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), or poly(4-hydroxy-L-proline ester).

In some embodiments, nanoparticles include acrylic polymers. As used herein, the term “acrylic polymer” refers to a polymer made up of acrylic acid monomers or derivatives or variants of acrylic acid. Monomers included in acrylic polymers may include, but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), and polycyanoacrylates.

### Lipids

In some embodiments, excipients include lipids. As used herein, the term “lipid” refers to members of a class of organic compounds that include fatty acids and various derivatives of fatty acids that are soluble in organic solvents, but not in water. Examples of lipids include, but are not limited to, fats, triglycerides oils, waxes, sterols (e.g. cholesterol, ergosterol, hopanoids, hydroxysteroids, phytosterol, and steroids), stearin, palmitin, triolein, fat-soluble vitamins (e.g., vitamins A, D, E, and K), monoglycerides (e.g. monolaurin, glycerol monostearate, and glyceryl hydroxystearate), diglycerides (e.g. diacylglycerol), phospholipids, glycerophospholipids (e.g., phosphatidic acid, phosphatidylethanolamine, phosphatidylcholine, phosphatidylserine, phosphoinositides), sphingolipids (e.g., sphingomyelin), and phosphosphingolipids. In some embodiments, lipids may include, but are not limited to, any of those listed (e.g., fats and fatty acids) in Table 1, above.

In some embodiments, lipid excipients include amphiphilic lipids (e.g., phospholipids). As used herein, the term “amphiphilic lipid” refers to a class of lipids with both hydrophilic and hydrophobic domains. Amphiphilic lipids may be used to prepare vesicles as these molecules typically form layers along water:lipid interfaces. Non-limiting examples of amphiphilic lipids include, but are not limited to, phospholipids, phosphatidylcholines, phosphatidylethanolamines, palmitoyl-oleoyl-phosphatidylethanolamine (POPE), phosphatidylserines, phosphotidylglycerols, lysophospholipids such as lysophosphatidylethanolamines, mono-oleoyl-phosphatidylethanolamine (MOPE), mono-myristoyl-phosphatidylethanolamine (MMPE), lysolipids, mono-oleoyl-phosphatidic acid (MOPA), mono-oleoyl-phosphatidylserine (MOPS), mono-oleoyl-phosphatidylglycerol (MOPG), palmitoyloleoyl phosphatidylcholine, lysophosphatidylethanolamine, dipalmitoylphosphatidylcholine, dioleoylphosphatidylcholine; distearoylphosphatidylcholine, dilinoleoylphosphatidylcholine, 1,2-dioleoyl-sn-glycero-3-phosphocholine (DOPC), 1,2-dioleoyl-sn-glycero-3-phosphoethanolamine (DOPE), 1,2-dilauroyl-sn-glycero-3-phosphocholine (DLPC), 1,2-dimyristoyl-sn-glycero-3-phosphocholine (DMPC) phosphatidylinositol, phosphatidic acid, palmitoyloleoyl phosphatidylcholine, lysophosphatidylethanolamines, monoglycerides, diglycerides, triglycerides.

### Lipid Vesicles

In some embodiments, excipients may include lipid vesicles or components of lipid vesicles. As used herein, the term “lipid vesicle” refers to a particle enveloped by an amphiphilic lipid membrane. Examples of lipid vesicles include, but are not limited to, liposomes, lipoplexes, and lipid nanoparticles. SBPs may include lipid vesicles as cargo or payloads. In some embodiments, SBPs are or encompassed by lipid vesicles. Such lipid vesicles may be used to deliver SBPs as a payload. Such SBPs may themselves include cargo or payload. As used herein, the term “liposome” refers generally to any vesicle that includes a phospholipid bilayer and aqueous core. Liposomes may be artificially prepared and may be used as delivery vehicles. Liposomes can be of different sizes. Multilamellar vesicles (MLVs) may be hundreds of nanometers in diameter and contain two or more concentric bilayers separated by narrow aqueous compartments. Small unicellular vesicles (SUVs) may be smaller than 50 nm in diameter. Large unilamellar vesicles (LUVs) may be between 50 and 500 nm in diameter. Liposomes may include opsonins or ligands to improve liposome attachment to unhealthy tissue or to activate events (e.g., endocytosis). Liposome core pH may be modulated to improve payload delivery. In some embodiments, lipid vesicle excipients may include, but are not limited to, any of those listed in Table 1, above.

In some embodiments, liposomes may include 1,2-dioleyloxy-N,N-dimethylaminopropane (DODMA) liposomes, DiLa2 liposomes (Marina Biotech, Bothell, Wash.), 1,2-dilinoleyloxy-3-dimethylaminopropane (DLin-DMA) liposomes, 2,2-dilinoleyl-4-(2-dimethylaminoethyl)-[1,3]-dioxolane (DLin-KC2-DMA) liposomes, and MC3 liposomes (e.g., see US Publication Number US20100324120, the contents of which are herein incorporated by reference in their entirety). In some embodiments, liposomes may include small molecule drugs (e.g., DOXIL® from Janssen Biotech. Inc., Horsham, Pa.).

Liposomes may be formed from the synthesis of stabilized plasmid-lipid particles (SPLP) or stabilized nucleic acid lipid particle (SNALP) that have been previously described and shown to be suitable for delivery of oligonucleotides in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132). These liposomes are designed for the delivery of DNA. RNA, and other oligonucleotide constructs, and they may be adapted for the delivery of SBPs with oligonucleotides. These liposome formulations may be composed of 3 to 4 lipid components in addition to SBPs. As an example, a liposome may contain 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, SBPs may be encapsulated within liposomes and/or contained in an encapsulated aqueous liposome core. In another embodiment, SBPs may be formulated in an oil-in-water emulsion where the emulsion particle comprises an oil core and a cationic lipid which can interact with SBPs, anchoring them to emulsion particles (e.g., see International Publication. Number WO2012006380, the contents of which are herein incorporated by reference in their entirety. In another embodiment, SBPs may be formulated in lipid vesicles which may have crosslinks between functionalized lipid bilayers (e.g., see United States Publication Number US20120177724, the contents of which are herein incorporated by reference in their entirety).

In some embodiments, lipid vesicles may include cationic lipids selected from one or more of formula CLI-CLXXIX of International Publication Number WO2008103276; formula CLI-CLXXIX of U.S. Pat. No. 7,893,302; formula CLI-CLXXXXII of U.S. Pat. No. 7,404,969; and formula 1-VI of United States Publication Number US20100036115, the contents of each of which are herein incorporated by reference in their entirety. As non-limiting examples, cationic lipids may be selected from (20Z,23Z)-N,N-dimethylnonacosa-20,23-dien-10-amine, (17Z,20Z)-N,N-dimethylhexacosa-17,20-dien-9-amine, (1Z,19Z)-N,N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimethyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-7-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethylheptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1-amine, N,N-dimethyl-1-[(1S,2R)-2-octylcyclopropyl]heptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{1[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine, (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxyl-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, (11E,20Z,23Z)-N,N-dimethylnonacosa-11,20,2-trien-10-amine, or pharmaceutically acceptable salts or stereoisomers thereof.

In some embodiments, lipids may be cleavable lipids. Such lipids may include any of those described in International Publication Number WO2012170889, the contents of which are herein incorporated by reference in their entirety. In some embodiments, SBPs may be formulated with at least one of the PEGylated lipids described in International Publication Number WO2012099755, the contents of which are herein incorporated by reference in their entirety.

In some embodiments, excipients include lipid nanoparticles. As used herein, the term “lipid nanoparticle” or “LNP” refers to a tiny colloidal particle of solid lipid and surfactant, typically ranging in size of from about 10 nm in diameter to about 1000 nm in diameter. LNPs may contain PEG-DMG 2000 (1,2-dimyristoyl-sn-glycero-3-phosphoethanolamine-N-[methoxy(polyethylene glycol)-2000). In some embodiments, LNPs may contain PEG-DMG 2000, a cationic lipid known in the art and at least one other component. LNPs may contain PEG-DMG 2000, a cationic lipid known in the art, DSPC and cholesterol. As a non-limiting example, LNPs may contain PEG-DMG 2000, DLin-DMA, DSPC, and cholesterol.

In some embodiments, excipients may include DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes, and hyaluronan-coated liposomes (Quiet Therapeutics, Israel).

In some embodiments, excipients may include lipidoids. As used herein, the term “lipidoid” refers to any non-lipid material that mimics lipid properties. The synthesis of lipidoids may be carried out as described by others (e.g., see Mahon et al., Bioconjug Chem. 2010 21:1448-1454; Schroeder et al., J Intern Med. 2010 267:9-21; Akine et al., Nat Biotechnol. 2008 26:561-569; Love et al., Proc Nat Acad Sci USA. 2010 107:1864-1869; and Siegwart et al., Proc Natl Acad Sci USA. 2011 108:12996-3001, the contents of each of which are herein incorporated by reference in their entireties). Lipidoids may be included in complexes, micelles, liposomes, or particles. In some embodiments, SBPs may include lipidoids.

In some embodiments, lipidoids may be combined with lipids to form particles. Such lipids may include cholesterol. Some lipidoids may be combined with PEG (e.g., C14 alkyl chain length). As another example, formulations with certain lipidoids, include, but are not limited to, C12-200 and may contain a combination of lipidoid, disteroylphosphatidyl choline, cholesterol, and PEG-DMG.

### Coating Agents

In some embodiments, excipients may include coating agents. Polymers are commonly used as coating agents, and may be layered over SBPs. Non-limiting examples of polymers for use as coating agents include polyethylene glycol, methylcellulose, hypromellose, ethylcellulose, gelatin, hydroxypropyl cellulose, titanium dioxide, zein, poly(alkyl)(meth)acrylate, poly(ethylene-co-vinyl acetate), and combinations thereof. In some embodiments, coating agents may include one or more compounds listed in Table 1, above.

### Bulking Agents

In some embodiments, excipients include bulking agents. As used herein, the term “bulking agent” refers to a substance that adds weight and volume to a composition. Examples of bulking agents include, but are not limited to, lactose, sorbitol, sucrose, mannitol, lactose USP, Starch 1500, microcrystalline cellulose, Avicel, dibasic calcium phosphate dehydrate, sucrose, tartaric acid, citric acid, fumaric acid, succinic acid, malic acid, polyvinylpyrrolidone, copolymers of vinylpyrrolidone and vinylacetate, hydroxypropylcellulose, hydroxyethylcellulose, hydroxypropylmethylcellulose, polyvinyl alcohol, polyethylene glycol, acacia, sodium carboxymethylcellulose, and combinations thereof. In some embodiments, bulking agents may include any of those presented in Table 1, above.

### Lubricants

In some embodiments, excipients may include lubricants. As used herein, the term “lubricant” refers to any substance used to reduce friction between two contacting materials. Lubricants may be natural or synthetic. Lubricants may comprise oils, lipids, microspheres, polymers, water, aqueous solutions, liposomes, solvents, alcohols, micelles, stearate salts, alkali, alkaline earth, and transition metal salts thereof (e.g., calcium, magnesium, or zinc), stearic acid, polyethylene oxide, talc, hydrogenated vegetable oil, and vegetable oil derivatives, fumed silica, silicones, high molecular weight polyalkylene glycol (e.g. high molecular weight polyethylene glycol), monoesters of propylene glycol, saturated fatty acids containing about 8-22 carbon atoms and preferably 16-20 carbon atoms, and any other component known to one skilled in the art. Other examples of lubricants include, but are not limited to, hyaluronic acid, magnesium stearate, calcium stearate, and lubricin. In some embodiments, lubricant excipients may include any of those presented in Table 1, above.

### Sweeteners and Colorants

In some embodiments, excipients may include sweeteners and/or colorants. As used herein, a “sweetener” refers to a substance that adds a sweet taste to or improves the sweetness of a composition. Sweeteners may be natural or artificial. Non-limiting examples of sweeteners include glucose, aspartame, sucralose, neotame, acesulfame potassium, saccharin, advantame, cyclamates, sorbitol, xylitol, lactitol, xylose, stevia, lead acetate, mogrosides, brazzein, curculin, erythritol, glycyrrhizin, glycerol, hydrogenated starte hydrolysates, inulin, ismalt, isomaltooligosaccharide, isomaltulose, mabinlin, maltodextrin, miraculin, monantin, osladin, pentadin, polydextrose, psicose, tagatose, thaumatin, mannitol, lactose, and sucrose. In some embodiments, sweetener excipients may include any of those presented in Table 1, above.

As used herein, the term “colorant” refers to any substance that adds color to a composition (e.g., a dye). Non-limiting examples of colorants include dyes, inks, pigments, food coloring, turmeric, titanium dioxide, caretinoids (e.g., bixin, β-carotene, apocarotenals, canthaxanthin, saffron, crocin, capsanthin and capsorubin occurring in paprika ole-oresin, lutein, astaxanthin, rubixanthin, violaxanthin, rhodoxanthin, lycopene, and derivatives thereof), and FD&C colorants [e.g., FD&C Blue No. 1 (brilliant blue FCF); FD&C Blue No. 2 (indigotine); FD&C Green No. 3 (fast green FCF); FD&C Red No. 40 (allura red AC) FD&C Red No. 3 (erythrosine); FD&C Yellow No. 5 (tartrazine); and FD&C Yellow No. 6 (sunset yellow)]. In some embodiments, colorant excipients may include any of those presented in Table 1, above.

### Preservatives

In some embodiments, excipients may include preservatives. As used herein a “preservative” is any substance that protects against decay, decomposition, or spoilage. Preservatives may be natural or synthetic. They may be antimicrobial preservatives, which inhibit the growth of bacteria or fungi, including mold, or antioxidants such as oxygen absorbers, which inhibit the oxidation of food constituents. Common antimicrobial preservatives include calcium propionate, sodium nitrate, sodium nitrite, sulfites (sulfur dioxide, sodium bisulfite, potassium hydrogen sulfite, etc.) and disodium EDTA. Antioxidants include BHA and BHT. Other preservatives include formaldehyde (usually in solution), glutaraldehyde (kills insects), vitamin A, vitamin C, vitamin E, selenium, amino acids, methyl paraben, propyl paraben, potassium sorbate, sodium chloride, ethanol, phenol, m-cresol, p-cresol, o-cresol, chlorocresol, benzyl alcohol, phenylmercuric nitrite, phenoxyethanol, methylchloroisothiazolinone, chlorobutanol, magnesium chloride (e.g., hexahydrate), alkylparaben (methyl, ethyl, propyl, butyl and the like), benzalkonium chloride, benzethonium chloride, sodium dehydroacetate, thimerosal, and combinations thereof. In some embodiments, preservative excipients may include any of those presented in Table 1, above.

### Flowability Agents

In some embodiments, excipients may include flowability agents. As used herein, the term “flowability agent” refers to a substance used to reduce viscosity and/or aggregation in a composition. Flowability agents are particularly useful for the formulation of powders, particles, solutions, gels, polymers, and any other form of matter capable of flow from one area to another. Flowability agents have been used to improve powder flowability for the manufacture of therapeutics, as taught in Morin et al. (2013) AAPS PharmSciTech 14(3):1158-1168, the contents of which are herein incorporated by reference in their entirety. In some embodiments, flowability agents are used to modulate SBP viscosity. In some embodiments, flowability agents may be lubricants. Non-limiting examples of flowability agents include magnesium stearate, stearic acid, hydrous magnesium silicate, and any other lubricant used to promote flowability known to one skilled in the art. In some embodiments, flowability agent excipients may include any of those presented in Table 1, above.

### Gelling Agents

In some embodiments, excipients may include gelling agents. As used herein, the term “gelling agent” refers to any substance that promotes viscosity and/or polymer cross-linking in compositions. Non-limiting examples of gelling agents include glycerol, glycerophosphate, sorbitol, hydroxyethyl cellulose, carboxymethyl cellulose, triethylamine, triethanolamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol formal, hydroxypropyl beta-cyclodextrin, kolliphor 124, kolliphor 181, kolliphor 188, kolliphor 407, kolliphor EL (cremaphor EL), cremaphor RH 40, cremaphor RH 60, d-alpha-tocopherol, PEG 1000 succinate, polysorbate 20, polysorbate 80, solutol HS 15, sorbitan monooleate, poloxamer-407, poloxamer-188, Labrafil M-1944CS, Labrafil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil, beeswax, d-alpha-tocopherol, oleic acid, medium-chain mono- and diglycerides, alpha-cyclodextrin, beta-cyclodextrin, hydroxypropyl-beta-cyclodextrin, sulfo-butylether-beta-cyclodextrin, hydrogenated soy phosphatidylcholine, distearoylphosphatidylglycerol, L-alpha-dimyristoylphosphatidylcholine, L-alphadimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate-SO, polyvinyl pyrrolidone, polyvinyl pyrrolidone-12, polyvinyl pyrrolidone-17, propylene carbonate, propylene glycol, solutol HS 15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, tetrahydrofuran, and glycerin. Additional examples of gelling agents include acacia, alginic acid, bentonite, CARBOPOLS® (also known as carbomers), carboxymethyl cellulose, ethylcellulose, gelatin, hydroxy ethyl cellulose, hydroxypropyl cellulose, magnesium aluminum silicate, methylcellulose, poloxamers, polyvinyl alcohol, sodium alginate, tragacanth, and xanthan gum. In some embodiments, gelling agent excipients may include any of those presented in Table 1, above.

PEGs which may be used as gelling agents and/or excipients may be selected from a variety of chain lengths and molecular weights. These compounds are typically prepared through ethylene oxide polymerization. In some embodiments, PEGs may have a molecular weight of from about 300 g/mol to about 100,000 g/mol. In some embodiments, PEGs may have a molecular weight of from about 3600 g/mol to about 4400 g/mol. In some embodiments, PEGs with a molecular weight of from about 300 g/mol to about 3000 g/mol, from about 350 g/mol to about 3500 g/mol, from about 400 g/mol to about 4000 g/mol, from about 450 g/mol to about 4500 g/mol, from about 500 g/mol to about 5000 g/mol, from about 550 g/mol to about 5500 g/mol, from about 600 g/mol to about 6000 g/mol, from about 650 g/mol to about 6500 g/mol, from about 700 g/mol to about 7000 g/mol, from about 750 g/mol to about 7500 g/mol, from about 800 g/mol to about 8000 g/mol, from about 850 g/mol to about 8500 g/mol, from about 900 g/mol to about 9000 g/mol, from about 950 g/mol to about 9500 g/mol, from about 1000 g/mol to about 10000 g/mol, from about 1100 g/mol to about 12000 g/mol, from about 1200 g/mol to about 14000 g/mol, from about 1300 g/mol to about 16000 g/mol, from about 1400 g/mol to about 18000 g/mol, from about 1500 g/mol to about 20000 g/mol, from about 1600 g/mol to about 22000 g/mol, from about 1700 g/mol to about 24000 g/mol, from about 1800 g/mol to about 26000 g/mol, from about 1900 g/mol to about 28000 g/mol, from about 2000 g/mol to about 30000 g/mol, from about 2200 g/mol to about 35000 g/mol, from about 2400 g/mol to about 40000 g/mol, from about 2600 g/mol to about 45000 g/mol, from about 2800 g/mol to about 50000 g/mol, from about 3000 g/mol to about 55000 g/mol, from about 10000 g/mol to about 60000 g/mol, from about 13000 g/mol to about 65000 g/mol, from about 16000 g/mol to about 70000 g/mol, from about 19000 g/mol to about 75000 g/mol, from about 22000 g/mol to about 80000 g/mol, from about 25000 g/mol to about 85000 g/mol, from about 28000 g/mol to about 90000 g/mol, from about 31000 g/mol to about 95000 g/mol, or from about 34000 g/mol to about 100000 g/mol are utilized.

### Demulcents

In some embodiments, excipients may include demulcents. As used herein, the term “demulcent” refers to a substance that relieves irritation or inflammation of the mucous membranes by forming a protective film. Demulcents may include non-polymeric demulcents and polymer demulcents. Non-limiting examples of non-polymeric demulcents include glycerin, gelatin, propylene glycol, and other non-polymeric diols and glycols. Non-limiting examples of polymer demulcents include polyvinyl alcohol (PVA), povidone or polyvinyl pyrrolidone (PVP), cellulose derivatives, polyethylene glycol (e.g., PEG 300, PEG 400), polysorbate 80, and dextran (e.g., dextran 70). Specific cellulose derivatives may include hydroxypropyl methyl cellulose, carboxymethyl cellulose, carboxymethylcellulose sodium, methyl cellulose, hydroxyethyl cellulose, hypromellose, and cationic cellulose derivatives.

### Formats

SBPs may include or be prepared to conform to a variety of formats. In some embodiments, such formats include formulations of processed silk with various excipients and/or cargo. In some embodiments, SBP formats include, but are not limited to, adhesives, capsules, coatings, cocoons, combs, cones, cylinders, discs, emulsions, fibers, films, foams, gels, grafts, hydrogels, implants, mats, membranes, microspheres, nanofibers, nanoparticles, nanospheres, nets, organogels, particles, patches, powders, rods, scaffolds, sheets, solids, solutions, sponges, sprays, spuns, suspensions, tablets, threads, tubes, vapors, and yarns. In some embodiments, the formats are formulated with a therapeutic agent.

### Formulations

In some embodiments, SBPs may be formulations. As used herein, the term “formulation” refers to a mixture of two or more components or the process of preparing such mixtures. In some embodiments, the formulations are low cost and eco-friendly. In some embodiments, the preparation or manufacturing of formulations is low cost and eco-friendly. In some embodiments, the preparation or manufacturing of formulations is scalable. In some embodiments, SBPs are prepared by extracting silk fibroin via degumming silk yarn. In some embodiments, the yarn is medical grade. In some embodiments the yarn may be silk sutures. The extracted silk fibroin may then be dissolved in a solvent (e.g. water, aqueous solution, organic solvent). The dissolved silk fibroin may then be dried (e.g., oven dried, air dried, or freeze-dried). In some embodiments, dried silk fibroin is formed into formats described herein. In some embodiments, that format is a solution. In some embodiments, that format is a powder. In some embodiments, formulations include one or more excipients, carriers, additional components, and/or therapeutic agents to generate SBPs. In some embodiments, formulations of processed silks are prepared during the manufacture of SBPs.

Formulation components and/or component ratios may be modulated to affect one or more SBP properties, effects, and/or applications. Variations in the concentration of silk fibroin, choice of excipient, the concentration of excipient, the osmolarity of the formulation, and the method of formulation represent non-limiting examples of differences in formulation that may alter properties, effects, and applications of SBPs. In some embodiments, the formulation of SBPs may modulate their physical properties. Examples of physical properties include solubility, density, and thickness. In some embodiments, the formulation of SBPs may modulate their mechanical properties. Examples of mechanical properties that may be modulated include, but are not limited to, mechanical strength, tensile strength, elongation capabilities, elasticity, compressive strength, stiffness, shear strength, toughness, torsional stability, temperature stability, moisture stability, viscosity, and reeling rate.

### Cargo

In some embodiments, SBPs are or include cargo. As used herein, the term “cargo” refers to any substance that is embedded in, enclosed within, attached to, or otherwise associated with a carrier. SBPs may be carriers for a large variety of cargo. Such cargo may include, but are not limited to, compounds, compositions, therapeutic agents, biological agents, materials, cosmetics, devices, agricultural compositions, particles, lipids, liposomes, sweeteners, colorants, preservatives, carbohydrates, small molecules, supplements, tranquilizers, ions, metals, minerals, nutrients, pesticides, herbicides, fungicides, and cosmetics.

In some embodiments, the cargo is or includes a payload. As used herein, the term “payload” refers to cargo that is delivered from a source or carrier to a target. Payloads may be released from SBPs, where SBPs serve as a carrier. Where SBPs are the payload, the SBPs may be released from a source or carrier. In some embodiments, payloads remain associated with carriers upon delivery. Payloads may be released in bulk or may be released over a period of time, also referred to herein as the “delivery period.” In some embodiments, payload release is by way of controlled release. As used herein, the term “controlled release” refers to distribution of a substance from a source or carrier to a surrounding area, wherein the distribution occurs in a manner that includes or is affected by some manipulation, some property of the carrier, or some carrier activity.

In some embodiments, controlled release may include a steady rate of release of payload from carrier. In some embodiments, payload release may include an initial burst, wherein a substantial amount of payload is released during an initial release period followed by a period where less payload is released. In some embodiments, release rate slows over time. Payload release may be measured by assessing payload concentration in a surrounding area and comparing to initial payload concentration or remaining payload concentration in a carrier or source area. Payload release rate may be expressed as a quantity or mass of payload released over time (e.g., mg/min). Payload release rate may be expressed as a percentage of payload released from a source or carrier over a period of time (e.g., 5%/hour). Controlled release of a payload that extends the delivery period is referred to herein as “sustained release.” Sustained release may include delivery periods that are extended over a period of hours, days, months, or years.

75 Some controlled release may be mediated by interactions between payload and carrier. Some controlled release is mediated by interactions between payload or carrier with surrounding areas where payload is released. With sustained payload release, payload release may be slowed or prolonged due to interactions between payload and carrier or payload and surrounding areas where payload is released. Payload release from SBPs may be controlled by SBP viscosity. Where the SBP includes processed silk gel, gel viscosity may be adjusted to modulate payload release.

In some embodiments, payload delivery periods may be from about 1 second to about 20 seconds, from about 10 seconds to about 1 minute, from about 30 seconds to about 10 minutes, from about 2 minutes to about 20 minutes, from about 5 minutes to about 30 minutes, from about 15 minutes to about 1 hour, from about 45 minutes to about 2 hours, from about 90 minutes to about 5 hours, from about 3 hours to about 20 hours, from about 10 hours to about 50 hours, from about 24 hours to about 100 hours, from about 48 hours to about 2 weeks, from about 72 hours to about 4 weeks, from about 1 week to about 3 months, from about 1 month to about 6 months, from about 3 months to about 1 year, from about 9 months to about 2 years, or more than 2 years.

In some embodiments, payload release may be consistent with near zero-order kinetics. In some embodiments, payload release may be consistent with first-order kinetics. In some embodiments, payload release may be modulated based on the density, loading, molecular weight, and/or concentration of the payload. Where the carrier is an SBP, payload release may be modulated by one or more of SBP drying method, silk fibroin molecular weight, and silk fibroin concentration.

In some embodiments, SBPs maintain and/or improve cargo stability, purity, and/or integrity. For example, SBPs may be used to protect therapeutic agents or macromolecules during lyophilization. The maintenance and/or improvement of stability during lyophilization may be determined by comparing SBP cargo stability to formulations lacking processed silk or to standard formulations in the art.

### Viscosity

In some embodiments, SBPs may be formulated to modulate SBP viscosity. As used herein, the term “viscosity” refers to a measure of a material's resistance to flow. The viscosity of a composition (e.g., a gel, e.g., hydrogel or organogel) provided herein can be determined using a rotational viscometer or rheometer. Additional methods for determining the viscosity of a composition (e.g., gel, e.g., hydrogel or organogel) and other properties of the gel are known in the art. In some embodiments, SBP viscosity may be altered by the incorporation of an excipient that is a gelling agent. In some embodiments, the identity of the excipient (e.g. PEG or poloxamer) may be altered to tune the viscosity of SBPs. In some embodiments, the viscosity of SBPs may be tuned for the desired application (e.g. tissue engineering scaffold, drug delivery system, surgical implant, etc.). In some embodiments, the processed silk preparations may shear thin or display shear thinning properties. As used herein, the term “shear thinning” refers to a decrease in viscosity at increasing shear rates. As used herein, the term “shear rate” refers to the rate of change in the ratio of displacement of material upon the application of a shear force to the height of the material. This ratio is also known as strain.

### Stress Resistance

In some embodiments, SBPs may be formulated to modulate SBP resistance to stress. Resistance to stress may be measured using one or more rheological measurements. Such measurements may include, but are not limited to tensile elasticity, shear or rigidity, volumetric elasticity, and compression. Additional rheological measurements and properties may include any of those taught in Zhang et al. (2017) Fiber and Polymers 18(10):1831-1840; McGill et al. (2017) Acta Biomaterialia 63:76-84; and Choi et al. (2015) In-Situ Gelling Polymers. Series in BioEngineering doi. 10.1007/978-981-287-152-7_2, the contents of each of which are herein incorporated by reference in their entirety. In some embodiments, stress resistance may be modulated through incorporation of excipients (e.g., PEG or poloxamer). In some embodiments, SBP stress-resistance properties may be modulated to suit a specific application (e.g., tissue engineering scaffold, drug delivery system, surgical implant, etc.).

### Concentrations and Ratios of SBP Components

SBPs may include formulations of processed silk with other components (e.g., excipients and cargo), wherein each SBP component is present at a specific concentration, ratio, or range of concentrations or ratios, depending on SBP format and/or application. In some embodiments, the concentration of processed silk or other SBP component (e.g., excipient or cargo) is present in SBPs at a concentration of from about 0.01% (w/v) to about 1% (w/v), from about 0.05% (w/v) to about 2% (w/v), from about 1% (w/v) to about 5% (w/v), from about 2% (w/v) to about 10% (w/v), from about 4% (w/v) to about 16% (w/v), from about 5% (w/v) to about 20% (w/v), from about 8% (w/v) to about 24% (w/v), from about 10% (w/v) to about 30% (w/v), from about 12% (w/v) to about 32% (w/v), from about 14% (w/v) to about 34% (w/v), from about 16% (w/v) to about 36% (w/v), from about 18% (w/v) to about 38% (w/v), from about 20% (w/v) to about 40% (w/v), from about 22% (w/v) to about 42% (w/v), from about 240% (w/v) to about 44% (w/v), from about 26% (w/v) to about 46% (w/v), from about 28% (w/v) to about 48% (w/v), from about 30% (w/v) to about 50% (w/v), from about 35% (w/v) to about 55% (w/v), from about 40% (w/v) to about 60% (w/v), from about 45% (w/v) to about 65% (w/), from about 50% (w/v) to about 70% (w/v), from about 55% (w/v) to about 75% (w/v), from about 60% (w/v) to about 80% (w/v), from about 65% (w/v) to about 85% (w/v), from about 70% (w/v) to about 90% (w/v), from about 75% (w/v) to about 95% (w/v), from about 80% (w/v) to about 96% (w/v), from about 85% (w/v) to about 97% (w/v), from about 90% (w/v) to about 98% (w/v), from about 95% (w/v) to about 99% (w/v), from about 96% (w/v) to about 99.2% (w/v), from about 97% (w/v) to about 99.5% (w/v), from about 98% (w/v) to about 99.8% (w/v), from about 99% (w/v) to about 99.9% (w/v), or greater than 99.9% (w/v).

In some embodiments, the concentration of processed silk or other SBP component (e.g., excipient or cargo) is present in SBPs at a concentration of from about 0.01% (v/v) to about 1% (v/v), from about 0.05% (v/v) to about 2% (v/v), from about 1% (v/v) to about 5% (v/v), from about 2% (v/v) to about 10% (v/v), from about 4% (v/v) to about 16% (v/v), from about 5% (v/v) to about 20% (v/v), from about 8% (v/v) to about 24% (v/v), from about 10% (v/v) to about 30% (v/v), from about 12% (v/v) to about 32% (v/v), from about 14% (v/v) to about 34% (v/v), from about 16% (v/v) to about 36% (v/v), from about 18% (v/v) to about 38% (v/v), from about 20% (v/v) to about 40% (v/v), from about 22% (v/v) to about 42% (v/v), from about 24% (v/v) to about 44% (v/v), from about 26% (v/v) to about 46% (v/v), from about 28% (v/v) to about 48% (v/v), from about 30% (v/v) to about 50% (v/v), from about 35% (v/v) to about 55% (v/v), from about 40% (v/v) to about 60% (v/v), from about 45% (v/v) to about 65% (v/v), from about 50% (v/v) to about 70% (v/v), from about 55% (v/v) to about 75% (v/v), from about 60% (v/v) to about 80% (v/v), from about 65% (v/v) to about 85% (v/v), from about 70% (v/v) to about 90% (v/v), from about 75% (v/v) to about 95% (v/v), from about 80% (v/v) to about 96% (v/v), from about 85% (v/v) to about 97% (v/v), from about 90° % (v/v) to about 98% (v/v), from about 95% (v/v) to about 99% (v/v), from about 96% (v/v) to about 99.2% (v/v), from about 97% (v/v) to about 99.5% (v/v), from about 98% (v/v) to about 99.8% (v/v), from about 99% (v/v) to about 99.9% (v/v), or greater than 99.9% (v/v).

In one embodiment, the concentration of processed silk or other SBP component (e.g., excipient or cargo) is present in SBPs at a concentration of 1% (w/v).

In one embodiment, the concentration of processed silk or other SBP component (e.g., excipient or cargo) is present in SBPs at a concentration of 2% (w/v).

In one embodiment, the concentration of processed silk or other SBP component (e.g., excipient or cargo) is present in SBPs at a concentration of 3% (w/v).

In one embodiment, the concentration of processed silk or other SBP component (e.g., excipient or cargo) is present in SBPs at a concentration of 4% (w/v).

In one embodiment, the concentration of processed silk or other SBP component (e.g., excipient or cargo) is present in SBPs at a concentration of 5% (w/v).

In one embodiment, the concentration of processed silk or other SBP component (e.g., excipient or cargo) is present in SBPs at a concentration of 6% (w/v).

In one embodiment, the concentration of processed silk or other SBP component (e.g., excipient or cargo) is present in SBPs at a concentration of 10% (w/v).

In one embodiment, the concentration of processed silk or other SBP component (e.g., excipient or cargo) is present in SBPs at a concentration of 20% (w/v).

In one embodiment, the concentration of processed silk or other SBP component (e.g., excipient or cargo) is present in SBPs at a concentration of 30% (w/v).

In one embodiment, the concentration of processed silk or other SBP component (e.g., excipient or cargo) is present in SBPs at a concentration of 16.7% (w/w).

In one embodiment, the concentration of processed silk or other SBP component (e.g., excipient or cargo) is present in SBPs at a concentration of 20% (w/w).

In one embodiment, the concentration of processed silk or other SBP component (e.g., excipient or cargo) is present in SBPs at a concentration of 23% (w/w).

In one embodiment, the concentration of processed silk or other SBP component (e.g., excipient or cargo) is present in SBPs at a concentration of 25% (w/w).

In one embodiment, the concentration of processed silk or other SBP component (e.g., excipient or cargo) is present in SBPs at a concentration of 27.3% (w/w).

In one embodiment, the concentration of processed silk or other SBP component (e.g., excipient or cargo) is present in SBPs at a concentration of 28.6% (w/w).

In one embodiment, the concentration of processed silk or other SBP component (e.g., excipient or cargo) is present in SBPs at a concentration of 33.3% (w/w).

In one embodiment, the concentration of processed silk or other SBP component (e.g., excipient or cargo) is present in SBPs at a concentration of 40% (w/w).

In one embodiment, the concentration of processed silk or other SBP component (e.g., excipient or cargo) is present in SBPs at a concentration of 50% (w/w).

In some embodiments, the concentration of processed silk or other SBP component (e.g., excipient or cargo) is present in SBPs at a concentration of from about 0.01% (w/w) to about 1% (w/w), from about 0.05% (w/w) to about 2% (w/w), from about 1% (w/w) to about 5% (w/w), from about 2% (w/w) to about 10% (w/w), from about 4% (w/w) to about 16% (w/w), from about 5% (w/w) to about 20% (w/w), from about 8% (w/w) to about 24% (w/w), from about 10% (w/v) to about 30% (w/v), from about 12% (w/w) to about 32% (w/w), from about 14% (w/w) to about 34% (w/w), from about 16% (w/w) to about 36% (w/w), from about 18% (w/w) to about 38% (w/w), from about 20% (w/w) to about 40% (w/w), from about 22% (w/v) to about 42% (w/w), from about 24% (w/w) to about 44% (w/w), from about 26% (w/w) to about 46% (w/w), from about 28% (w/w) to about 48% (w/w), from about 30% (w/v) to about 50% (w/w), from about 35% (w/w) to about 55% (w/w), from about 40% (w/w) to about 60% (w/w), from about 45% (w/w) to about 65% (w/w), from about 50% (w/w) to about 70% (w/w), from about 55% (w/w) to about 75% (w/w), from about 60% (w/w) to about 80% (w/w), from about 65% (w/w) to about 85% (w/w), from about 70% (w/w) to about 90% (w/w), from about 75% (w/w) to about 95% (w/w), from about 80% (w/w) to about 96% (w/w), from about 85% (w/w) to about 97% (w/w), from about 90% (w/w) to about 98% (w/w), from about 95% (w/w) to about 99% (w/w), from about 96% (w/w) to about 99.2% (w/w), from about 97% (w/w) to about 99.5% (w/w), from about 98% (w/w) to about 99.8% (w/w), from about 99% (w/w) to about 99.9% (w/w), or greater than 99.9% (w/w).

In some embodiments, the concentration of processed silk (e.g., silk fibroin) or other SBP component (e.g., excipient or cargo) is present in SBPs at a concentration of from about 0.01 pg/mL to about 1 pg/mL, from about 0.05 pg/mL to about 2 pg/mL, from about 1 pg/mL to about 5 pg/mL, from about 2 pg/mL to about 10 pg/mL, from about 4 pg/mL to about 16 pg/mL, from about 5 pg/mL to about 20 pg/mL, from about 8 pg/mL to about 24 pg/mL, from about 10 pg/mL to about 30 pg/mL, from about 12 pg/mL to about 32 pg/mL, from about 14 pg/mL to about 34 pg/mL, from about 16 pg/mL to about 36 pg/mL, from about 18 pg/mL to about 38 pg/mL, from about 20 pg/mL to about 40 pg/mL, from about 22 pg/mL to about 42 pg/mL, from about 24 pg/mL to about 44 pg/mL, from about 26 pg/mL to about 46 pg/mL, from about 28 pg/mL to about 48 pg/mL, from about 30 pg/mL to about 50 pg/mL, from about 35 pg/mL to about 55 pg/mL, from about 40 pg/mL to about 60 pg/mL, from about 45 pg/mL to about 65 pg/mL, from about 50 pg/mL to about 75 pg/mL, from about 60 pg/mL to about 240 pg/mL, from about 70 pg/mL to about 350 pg/mL, from about 80 pg/mL to about 400 pg/mL, from about 90 pg/mL to about 450 pg/mL, from about 100 pg/mL to about 500 pg/mL, from about 0.01 ng/mL to about 1 ng/mL, from about 0.05 ng/mL to about 2 ng/mL, from about 1 ng/mL to about 5 ng/mL, from about 2 ng/mL to about 10 ng/mL, from about 4 ng/mL to about 16 ng/mL, from about 5 ng/mL to about 20 ng/mL, from about 8 ng/mL to about 24 ng/mL, from about 10 ng/mL to about 30 ng/mL, from about 12 ng/mL to about 32 ng/mL, from about 14 ng/mL to about 34 ng/mL, from about 16 ng/mL to about 36 ng/mL, from about 18 ng/mL to about 38 ng/mL, from about 20 ng/mL to about 40 ng/mL, from about 22 ng/mL to about 42 ng/mL, from about 24 ng/mL to about 44 ng/mL, from about 26 ng/mL to about 46 ng/mL, from about 28 ng/mL to about 48 ng/mL, from about 30 ng/mL to about 50 ng/mL, from about 35 ng/mL to about 55 ng/mL, from about 40 ng/mL to about 60 ng/mL, from about 45 ng/mL to about 65 ng/mL, from about 50 ng/mL to about 75 ng/mL, from about 60 ng/mL to about 240 ng/mL, from about 70 ng/mL to about 350 ng/mL, from about 80 ng/mL to about 400 ng/mL, from about 90 ng/mL to about 450 ng/mL, from about 100 ng/mL to about 500 ng/mL, from about 0.01 μg/mL to about 1 μg/mL, from about 0.05 μg/mL to about 2 μg/mL, from about 1 μg/mL to about 5 μg/mL, from about 2 μg/mL to about 10 μg/mL, from about 4 μg/mL to about 16 μg/mL, from about 5 μg/mL to about 20 μg/mL, from about 8 μg/mL to about 24 μg/mL, from about 10 μg/mL to about 30 μg/mL, from about 12 μg/mL to about 32 μg/mL, from about 14 μg/mL to about 34 μg/mL, from about 16 μg/mL to about 36 μg/mL, from about 18 μg/mL to about 38 μg/mL, from about 20 μg/mL to about 40 μg/mL, from about 22 μg/mL to about 42 μg/mL, from about 24 μg/mL to about 44 μg/mL, from about 26 μg/mL to about 46 μg/mL, from about 28 μg/mL to about 48 μg/mL, from about 30 μg/mL to about 50 μg/mL, from about 35 μg/mL to about 55 μg/mL, from about 40 μg/mL to about 60 μg/mL, from about 45 μg/mL to about 65 μg/mL, from about 50 μg/mL to about 75 μg/mL, from about 60 μg/mL to about 240 μg/mL, from about 70 μg/mL to about 350 μg/mL, from about 80 μg/mL to about 400 μg/mL, from about 90 μg/mL to about 450 μg/mL, from about 100 μg/mL to about 500 μg/mL, from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 36 mg/mL, from about 18 mg/mL to about 38 mg/mL, from about 20 mg/mL to about 40 mg/mL, from about 22 mg/mL to about 42 mg/mL, from about 24 mg/mL to about 44 mg/mL, from about 26 mg/mL to about 46 mg/mL, from about 28 mg/mL to about 48 mg/mL, from about 30 mg/mL to about 50 mg/mL, from about 35 mg/mL to about 55 mg/mL, from about 40 mg/mL to about 60 mg/mL, from about 45 mg/mL to about 65 mg/mL, from about 50 mg/mL to about 75 mg/mL, from about 60 mg/mL to about 240 mg/mL, from about 70 mg/mL to about 350 mg/mL, from about 80 mg/mL to about 400 mg/mL, from about 90 mg/mL to about 450 mg/mL, from about 100 mg/mL to about 500 mg/mL, from about 0.01 g/mL to about 1 g/mL, from about 0.05 g/mL to about 2 g/mL, from about 1 g/mL to about 5 g/mL, from about 2 g/mL to about 10 g/mL, from about 4 g/mL to about 16 g/mL, or from about 5 g/mL to about 20 g/mL.

In one embodiment, the concentration of processed silk (e.g., silk fibroin) or other SBP component (e.g., excipient or cargo) is present in SBPs at a concentration of 5 mg/mL.

In one embodiment, the concentration of processed silk (e.g., silk fibroin) or other SBP component (e.g., excipient or cargo) is present in SBPs at a concentration of 2.5 mg/mL.

In one embodiment, the concentration of processed silk (e.g., silk fibroin) or other SBP component (e.g., excipient or cargo) is present in SBPs at a concentration of 1.25 mg/mL.

In one embodiment, the concentration of processed silk (e.g., silk fibroin) or other SBP component (e.g., excipient or cargo) is present in SBPs at a concentration of 0.625 mg/mL.

In one embodiment, the concentration of processed silk (e.g., silk fibroin) or other SBP component (e.g., excipient or cargo) is present in SBPs at a concentration of 0.3125 mg/mL.

In some embodiments, the concentration of processed silk (e.g., silk fibroin) or other SBP component (e.g., excipient or cargo) is present in SBPs at a concentration of from about 0.01 pg/kg to about 1 pg/kg, from about 0.05 pg/kg to about 2 pg/kg, from about 1 pg/kg to about 5 pg/kg, from about 2 pg/kg to about 10 pg/kg, from about 4 pg/kg to about 16 pg/kg, from about 5 pg/kg to about 20 pg/kg, from about 8 pg/kg to about 24 pg/kg, from about 10 pg/kg to about 30 pg/kg, from about 12 pg/kg to about 32 pg/kg, from about 14 pg/kg to about 34 pg/kg, from about 16 pg/kg to about 36 pg/kg, from about 18 pg/kg to about 38 pg/kg, from about 20 pg/kg to about 40 pg/kg, from about 22 pg/kg to about 42 pg/kg, from about 24 pg/kg to about 44 pg/kg, from about 26 pg/kg to about 46 pg/kg, from about 28 pg/kg to about 48 pg/kg, from about 30 pg/kg to about 50 pg/kg, from about 35 pg/kg to about 55 pg/kg, from about 40 pg/kg to about 60 pg/kg, from about 45 pg/kg to about 65 pg/kg, from about 50 pg/kg to about 75 pg/kg, from about 60 pg/kg to about 240 pg/kg, from about 70 pg/kg to about 350 pg/kg, from about 80 pg/kg to about 400 pg/kg, from about 90 pg/kg to about 450 pg/kg, from about 100 pg/kg to about 500 pg/kg, from about 0.01 ng/kg to about 1 ng/kg, from about 0.05 ng/kg to about 2 ng/kg, from about 1 ng/kg to about 5 ng/kg, from about 2 ng/kg to about 10 ng/kg, from about 4 ng/kg to about 16 ng/kg, from about 5 ng/kg to about 20 ng/kg, from about 8 ng/kg to about 24 ng/kg, from about 10 ng/kg to about 30 ng/kg, from about 12 ng/kg to about 32 ng/kg, from about 14 ng/kg to about 34 ng/kg, from about 16 ng/kg to about 36 ng/kg, from about 18 ng/kg to about 38 ng/kg, from about 20 ng/kg to about 40 ng/kg, from about 22 ng/kg to about 42 ng/kg, from about 24 ng/kg to about 44 ng/kg, from about 26 ng/kg to about 46 ng/kg, from about 28 ng/kg to about 48 ng/kg, fom about 30 ng/kg to about 50 ng/kg, from about 35 ng/kg to about 55 ng/kg, from about 40 ng/kg to about 60 ng/kg, from about 45 ng/kg to about 65 ng/kg, from about 50 ng/kg to about 75 ng/kg, from about 60 ng/kg to about 240 ng/kg, from about 70 ng/kg to about 350 ng/kg, from about 80 ng/kg to about 400 ng/kg, from about 90 ng/kg to about 450 ng/kg, from about 100 ng/kg to about 500 ng/kg, from about 0.01 μg/kg to about 1 μg/kg, from about 0.05 μg/kg to about 2 μg/kg, from about 1 μg/kg to about 5 μg/kg, from about 2 μg/kg to about 10 μg/kg, from about 4 μg/kg to about 16 μg/kg, from about 5 μg/kg to about 20 μg/kg, from about 8 μg/kg to about 24 μg/kg, from about 10 μg/kg to about 30 μg/kg, from about 12 μg/kg to about 32 μg/kg, from about 14 μg/kg to about 34 μg/kg, from about 16 μg/kg to about 36 μg/kg, from about 18 μg/kg to about 38 μg/kg, from about 20 μg/kg to about 40 μg/kg, from about 22 μg/kg to about 42 μg/kg, from about 24 μg/kg to about 44 μg/kg, from about 26 μg/kg to about 46 μg/kg, from about 28 μg/kg to about 48 μg/kg, from about 30 μg/kg to about 50 μg/kg, from about 35 μg/kg to about 55 μg/kg, from about 40 μg/kg to about 60 μg/kg, from about 45 μg/kg to about 65 μg/kg, from about 50 μg/kg to about 75 μg/kg, from about 60 μg/kg to about 240 μg/kg, from about 70 μg/kg to about 350 μg/kg, from about 80 μg/kg to about 400 μg/kg, from about 90 μg/kg to about 450 μg/kg, from about 100 μg/kg to about 500 μg/kg, from about 0.01 mg/kg to about 1 mg/kg, from about 0.05 mg/kg to about 2 mg/kg, from about 1 mg/kg to about 5 mg/kg, from about 2 mg/kg to about 10 mg/kg, from about 4 mg/kg to about 16 mg/kg, from about 5 mg/kg to about 20 mg/kg, from about 8 mg/kg to about 24 mg/kg, from about 10 mg/kg to about 30 mg/kg, from about 12 mg/kg to about 32 mg/kg, from about 14 mg/kg to about 34 mg/kg, from about 16 mg/kg to about 36 mg/kg, from about 18 mg/kg to about 38 mg/kg, from about 20 mg/kg to about 40 mg/kg, from about 22 mg/kg to about 42 mg/kg, from about 24 mg/kg to about 44 mg/kg, from about 26 mg/kg to about 46 mg/kg, from about 28 mg/kg to about 48 mg/kg, from about 30 mg/kg to about 50 mg/kg, from about 35 mg/kg to about 55 mg/kg, from about 40 mg/kg to about 60 mg/kg, from about 45 mg/kg to about 65 mg/kg, from about 50 mg/kg to about 75 mg/kg, from about 60 mg/kg to about 240 mg/kg, from about 70 mg/kg to about 350 mg/kg, from about 80 mg/kg to about 400 mg/kg, from about 90 mg/kg to about 450 mg/kg, from about 100 mg/kg to about 500 mg/kg, from about 0.01 g/kg to about 1 g/kg, from about 0.05 g/kg to about 2 g/kg, from about 1 g/kg to about 5 g/kg, from about 2 g/kg to about 10 g/kg, from about 4 g/kg to about 16 g/kg, or from about 5 g/kg to about 20 g/kg, from about 10 g/kg to about 50 g/kg, from about 15 g/kg to about 100 g/kg, from about 20 g/kg to about 150 g/kg, from about 25 g/kg to about 200 g/kg, from about 30 g/kg to about 250 g/kg, from about 35 g/kg to about 300 g/kg, from about 40 g/kg to about 350 g/kg, from about 45 g/kg to about 400 g/kg, from about 50 g/kg to about 450 g/kg, from about 55 g/kg to about 500 g/kg, from about 60 g/kg to about 550 g/kg, from about 65 g/kg to about 600 g/kg, from about 70 g/kg to about 650 g/kg, from about 75 g/kg to about 700 g/kg, from about 80 g/kg to about 750 g/kg, from about 85 g/kg to about 800 g/kg, from about 90 g/kg to about 850 g/kg, from about 95 g/kg to about 900 g/kg, from about 100 g/kg to about 950 g/kg, or from about 200 g/kg to about 1000 g/kg.

In some embodiments, SBPs may be formatted as a gel. Such gels may include hydrogels. In some embodiments, such hydrogels are formulated with therapeutic agents. Therapeutic agents may include a nonsteroidal anti-inflammatory drug (NSAID), for example, celecoxib.

### Appearance: Transparent, Opaque, Translucent

In some embodiments, the appearance of SBPs described in the present disclosure may be tuned for the application for which they were designed. In some embodiments, SBPs may be transparent. In some embodiments, SBPs may be translucent. In some embodiments, SBPs may be opaque. In some embodiments, SBP preparation methods may be used to modulate clarity, as taught in International Patent Application Publication No. WO2012170655, the contents of which are herein incorporated by reference in their entirety. In some embodiments, the incorporation of excipients may be used to tune the clarity of processed silk preparations. In some embodiments, the excipient is sucrose. In some embodiments, the sucrose may also increase protein reconstitution during lyophilization. In some embodiments, sucrose may improve processed silk hydrogel clarity (optically transparency). In some embodiments, optically transparent SBPs may be used for ocular applications, e.g., treatment of ocular conditions, diseases, and/or indications. In some embodiments, SBPs herein may be used to label products, as taught in International Patent Application Publication No. WO2009155397, the contents of which are herein incorporated by reference in their entirety. The transparency of SBPs, as well as other properties, may render resulting labels edible, biodegradable, and/or holographic.

pH

SBPs may have a pH from about 3 to about 10. In some embodiments, the pH is from about 3 to about 6, from about 6 to about 8, or from about 8 to about 10. In some embodiments, the pH of the SBP is about 7.4. In some embodiments, the pH of the SBP is 7.06. In some embodiments, the pH of the SBP is 7.15.

### Exemplary Formulations

In one embodiment, the SBP formulation may include 480 mb silk fibroin at a concentration of 3%, an excipient at a concentration of 10% and cargo at a concentration of 10%. The excipient cargo may be, but is not limited to, poloxamer-188 (P188) and PEG4k, and the cargo may be, celecoxib (CXB), bovine serum albumin (BSA), lysozyme or bevacizumab. The osmolarity of the SBP formulation may be the range of 290-320 mOsm/L.

In one embodiment, the SBP formulation may include 480 mb silk fibroin at a concentration of 3%, an excipient at a concentration of 20% and cargo at a concentration of 1%. The excipient cargo may be, but is not limited to, poloxamer-188 (P188) and PEG4k, and the cargo may be, celecoxib (CXB), bovine serum albumin (BSA), lysozyme or bevacizumab. The osmolarity of the SBP formulation may be the range of 290-320 mOsm/L.

In one embodiment, the SBP formulation may include 480 mb silk fibroin at a concentration of 3%, an excipient at a concentration of 50% and cargo at a concentration of 1%. The excipient cargo may be, but is not limited to, poloxamer-188 (P188) and PEG4k, and the cargo may be, celecoxib (CXB), bovine serum albumin (BSA), lysozyme or bevacizumab. The osmolarity of the SBP formulation may be the range of 290-320 mOsm/L.

In one embodiment, the SBP formulation may include 120 mb silk fibroin at a concentration of 2%, 3%, 4%, 5%, or 6%. The SBP formulation may include an excipient at a concentration of 40% and may be PEG300 or glycerol and/or cargo a concentration of 10%. The cargo may be, celecoxib (CXB), bovine serum albumin (BSA), lysozyme or bevacizumab. Additionally 0.2% polysorbate-80 and 22 mM phosphate buffer may be included in the formulation.

### Combinations

In some embodiments, SBPs are presented in a combinatorial format. A combinatorial format may consist of two or more different materials that have been combined to form a single composition. In some embodiments, two or more SBPs of different formats (e.g. rod, hydrogel etc.) are combined to form a single composition (e.g., see European Publication Number EP3212246, the contents of which are herein incorporated by reference in their entirety). In some embodiments, one or more SBP is combined with a different material (e.g. a polymer, a mat, a particle, a microsphere, a nanosphere, a metal, a scaffold, etc.) to form a single composition (e.g., see International Publication Number WO2017179069, the contents of which are herein incorporated by reference in their entirety. In some embodiments, combinatorial formats are prepared by formulating two or more SBPs of different formats as a single composition (e.g., see Kambe et al. (2017) Materials (Basel) 10(10):1153, the contents of which are herein incorporated by reference in their entirety). In some embodiments, combinatorial formats are prepared by formulating two or more SBPs of different formats, along with another material, as a single composition (e.g., see International Publication Number WO2017177281, the contents of which are herein incorporated by reference in their entirety). In some embodiments, combinatorial formats include adding one or more SBPs to a first SBP of a different format (e.g., see European Patent Number EP3212246, the contents of which are herein incorporated by reference in their entirety). In some embodiments, combinatorial formats include adding one or more SBPs to a first composition comprising a different material (e.g., see Jiang et al. (2017) J Biomater Sci Polym Ed 15:1-36, the contents of which are herein incorporated by reference in their entirety). In some embodiments, the combinatorial formats are prepared by adding one or more materials to one or more first formed SBPs (e.g., see Babu et al. (2017) J Colloid Interface Sci 513:62-72, the contents of which are herein incorporated by reference in their entirety).

### Distribution

SBP components may be distributed equally or unequally, depending on format and application. Non-limiting examples of unequal distribution include component localization in SBP regions or compartments, on SBP surfaces, etc. In some embodiments, components include cargo. Such cargo may include payloads, for example, therapeutic agents. In some embodiments, therapeutic agents are present on the surface of an SBP (e.g., see Han et al. (2017) Biomacromolecules 18(11):3776-3787; Ran et al. (2017) Biomacromolecules 18(11):3788-3801, the contents of each of which are herein incorporated by reference in their entirety). In some embodiments, components (e.g., therapeutic agents) are homogenously mixed with processed silk to generate a desired distribution (e.g., see United States Publication No. US20170333351; Sun et al. (2017) Journal of Materials Chemistry B 5:8770-8779; and Du et al. (2017) Nanoscale Res Lett 12(1):573, the contents of each of which are herein incorporated by reference in their entirety). In some embodiments, components (e.g., therapeutic agents) are encapsulated in SBPs (e.g., see Shi et al. (2017) Nanoscale 9:14520, the contents of which are herein incorporated by reference in their entirety).

### Solubility

In some embodiments, SBPs or components thereof are water soluble. The water solubility, along with the rate of degradation, of SBPs may modulate payload (e.g., therapeutic agent) release rate and/or release period. An increasing amount of payload may be released into surrounding medium as surrounding matrix dissolves (e.g., see International Publication Numbers WO2013126799 and WO2017165922; and U.S. Pat. No. 8,530,625, the contents of each of which are herein incorporated by reference in their entirety). Longer time periods required to dissolve SBPs or components thereof may result in longer release periods. In some embodiments, SBP solubility may be modulated in order to control the rate of payload release in the surrounding medium. The solubility of SBPs may be modulated via any method known to those skilled in the art. In some embodiments, SBP solubility may be modulated by altering included silk fibroin secondary structure (e.g., increasing β-sheet content and/or crystallinity). In some embodiments, SBP solubility may be modulated by altering SBP format. In some embodiments, SBP solubility and/or rate of degradation may be modulated to facilitate extended release of therapeutic agent payloads in vitro and/or in vivo.

### Coating Agents

In some embodiments, SBPs may be used as coating agents. As used herein, the term “coating agent” refers to a substance covering or used to cover an article, wherein the substance adheres to the article (also referred to herein as “coatings”). Coating agents may include, but are not limited to, processed silk, paints, lacquers, adhesives, surfactants, particles, liquids, metals, lipids, oils, proteins, plastics, polymers, insulations, films, and membranes. Coating agents may be used, for example, to coat cargo, payloads, devices, or device components. Coatings may be used to protect coated articles. Some coatings may be used to impart a desired property to the article coated (e.g., to provide a desired texture, flavor, hydrophobicity, etc.). In some embodiments. SBP coating agents are used as lubricants. Additional non-limiting examples of coating agents are listed in Table 1. In some embodiments, coating agents may include any of the excipients listed in Table 1.

### Rods

In some embodiments, SBPs are prepared as rods. As used herein when referring to processed silk preparations or SBPs, the term “rod” refers to an elongated format, typically cylindrical, that may have blunted or tapered ends. Rods may be suitable for implantation or similar administration methods as it may be possible to deliver rods by injection. Rods may also be obtained simply by passing suitably viscous processed silk preparations through a needle, cannula, tube, or opening. In some embodiments, rods are prepared by one or more of injection molding, heated or cooled extrusion, extrusion through a coating agent, milling with a therapeutic agent, and combining with a polymer followed by extrusion.

In some embodiments, SBP rods include processed silk (e.g., silk fibroin) rods. Some rods may include coterminous luminal cavities in whole or in part running through the rod. Rods may be of any cross-sectional shape, including, but not limited to, circular, square, oval, triangular, irregular, or combinations thereof.

In some embodiments, rods are prepared from silk fibroin preparations. The silk fibroin preparations may include lyophilized silk fibroin. The lyophilized silk fibroin may be dissolved in water to form silk fibroin solutions used in rod preparation. Silk fibroin solutions may be prepared as stock solutions to be combined with additional components prior to rod preparation. In some embodiments silk fibroin stock solutions have a silk fibroin concentration of between 10% (w/v) and 40% (w/v). In some embodiments, the silk fibroin stock solution for the preparation of silk fibroin rods has a concentration of at least 10% (w/v), at least 20% (w/v), at least 30% (w/v), at least 40% (w/v), or at least 50% (w/v).

In one embodiment, the silk fibroin stock solution has a concentration of 10% (w/v).

In one embodiment, the silk fibroin stock solution has a concentration of 20% (w/v).

In one embodiment, the silk fibroin stock solution has a concentration of 30% (w/v).

In one embodiment, the silk fibroin stock solution has a concentration of 40% (w/v).

In one embodiment, the silk fibroin stock solution has a concentration of 50% (w/v).

In some embodiments, silk fibroin stock solution prepared for rod formation are mixed with one or more other components intended to be include in the final processed silk rods. Examples of such other components include, but are not limited to, excipients, salts, therapeutic agents, biological agents, proteins, small molecules, and polymers. In some embodiments, processed silk rods may include between 20 to 55% (w/w) silk fibroin. In some embodiments, processed silk rods may include between 40 to 80% (w/w) therapeutic agent. In some embodiments, processed silk rods may include 35% (w/w) silk fibroin and 65% (w/w) therapeutic agent. In some embodiments, processed silk rods may include 30% (w/w) silk fibroin and 70% (w/w) therapeutic agent. In some embodiments, processed silk rods may include 40% (w/w) silk fibroin and 60% (w/w) therapeutic agent. In some embodiments, processed silk rods may include 26% (w/w) silk fibroin and 74% (w/w) therapeutic agent. In some embodiments, processed silk rods may include 37% (w/w) silk fibroin and 63% (w/w) therapeutic agent. In some embodiments, processed silk rods may include 33% (w/w) silk fibroin and 66% (w/w) therapeutic agent. In some embodiments, processed silk rods may include 51% (w/w) silk fibroin and 49% (w/w) therapeutic agent. In some embodiments, silk fibroin may be included at a concentration (w/w) of 0.01% to about 1%, from about 0.05% to about 2%, from about 0.1% to about 30%, from about 1% to about 5%, from about 2% to about 10%, from about 3% to about 15%, from about 4% to about 20%, from about 5% to about 25%, from about 6% to about 30%, from about 7% to about 35%, from about 8% to about 40%, from about 9% to about 45%, from about 10% to about 50%, from about 12% to about 55%, from about 14% to about 60%, from about 16% to about 65%, from about 18% to about 70%, from about 20% to about 75%, from about 22% to about 80%, from about 24% to about 85%, from about 26% to about 90%, from about 28% to about 95%, from about 30% to about 96%, from about 32% to about 97%, from about 34% to about 98%, from about 36% to about 98.5%, from about 38% to about 99%, from about 40% to about 99.5%, from about 42% to about 99.6%, from about 44% to about 99.7%, from about 46% to about 99.8%, or from about 50% to about 99.9%.

In some embodiments, processed silk rods are prepared by extrusion. As used herein, the term “extrusion” refers to a process by which a substance is forced through an opening, tube, or passage. In some embodiments, processed silk rods are formed by extruding processed silk preparations through a needle or cannula. Processed silk preparations used for rod formation may have varying levels of viscosity. Preparation viscosity may depend on the presence and/or identity of excipients present. In some embodiments, processed silk preparations may include compounds or compositions intended to be embedded in rods prepared by extrusion. Excipients, compounds, or compositions included in processed silk preparations used for extrusion may include, but are not limited to, salts, therapeutic agents, biological agents, proteins, small molecules, and polymers. Extrusion may be carried out manually or by an automated process.

In some embodiments, extrusion may be carried out using a syringe. The syringe may be fitted with a needle, tube, or cannula. The needle, tube, or cannula may have a sharpened end or a blunt end. The needle may have a diameter of from about 0.1 mm to about 0.3 mm, from about 0.2 mm to about 0.7 mm, from about 0.4 mm to about 1.1 mm, from about 0.6 mm to about 1.5 mm, from about 0.8 mm to about 1.9 mm, from about 1 mm to about 2.3 mm, from about 1.2 mm to about 2.7 mm, from about 1.6 mm to about 3.1 mm, or from about 2 mm to about 3.5 mm. Processed silk preparations may be used to fill tubes, wherein the processed silk preparations are incubated in the tubes for various periods of time under various conditions (e.g., various temperatures). In some embodiments, tubing filled with processed silk preparation may be incubated at 37° C. for from about 2 hours to about 36 hours or more. In some embodiments, processed silk filled tubing is incubated for 24 hours. In some embodiments, processed silk preparations remain in tubing after the 37° C. incubation. In some embodiments, processed silk preparations are removed from the tubing after the incubation at 37° C. Processed silk preparations removed from tubing may maintain a rod-shaped format. Such preparations may be dried after removal from tubing. In some embodiments, processed silk preparations may be encased in tubing while drying. Rods may be dried by one or more of freeze-drying, oven drying, and air drying. Some processed silk preparations may be removed tubing after drying.

Tubing used for extrusion may be composed of various materials. In some embodiments, tubing is made from one or more of silicone, polyetheretherketone (PEEK), polytetrafluoroethylene (PTFE), amorphous fluoroplastics, fluorinated ethylene propylene, perfluoroalkoxy copolymers, ethylene-tetrafluoroethylene, polyolefins, and nylon.

In some embodiments, rods may have a diameter of from about 0.05 μm to about 10 μm, from about 1 μm to about 20 μm, from about 2 μm to about 30 μm, from about 5 μm to about 40 μm, from about 10 μm to about 50 μm, from about 20 μm to about 60 μm, from about 30 μm to about 70 μm, from about 40 μm to about 80 μm, from about 50 μm to about 90 μm, from about 0.05 mm to about 2 mm, from about 0.1 mm to about 3 mm, from about 0.2 mm to about 4 mm, from about 0.5 mm to about 5 mm, from about 1 mm to about 6 mm, from about 2 mm to about 7 mm, from about 5 mm to about 10 mm, from about 8 mm to about 16 mm, from about 10 mm to about 50 mm, from about 20 mm to about 100 mm, from about 40 mm to about 200 mm, from about 60 mm to about 300 mm, from about 80 mm to about 400 mm, from about 250 mm to about 750 mm, or from about 500 mm to about 1000 mm. In some embodiments, rods include a diameter of at least 0.5 μm, at least 1 μm at least 10 μm, at least 100 μm, at least 500 μm, at least 1 mm, at least 10 mm, or at least 100 mm. In one embodiment, the rods have a diameter of 1 mm. In another embodiment, the rods have a diameter of 0.5 mm. In another embodiment, the rods have a diameter of 400 um. In another embodiment, the rods have a diameter of 430 um.

In some embodiments, the rods described herein may have a density of from about 0.01 μg/mL to about 1 μg/mL, from about 0.05 μg/mL to about 2 μg/mL, from about 1 μg/mL to about 5 μg/mL, from about 2 μg/mL to about 10 μg/mL, from about 4 μg/mL to about 16 μg/mL, from about 5 μg/mL to about 20 μg/mL, from about 8 μg/mL to about 24 μg/mL, from about 10 μg/mL to about 30 μg/mL, from about 12 μg/mL to about 32 μg/mL, from about 14 μg/mL to about 34 μg/mL, from about 16 μg/mL to about 36 μg/mL, from about 18 μg/mL to about 38 μg/mL, from about 20 μg/mL to about 40 μg/mL, from about 22 μg/mL to about 42 μg/mL, from about 24 μg/mL to about 44 μg/mL, from about 26 μg/mL to about 46 μg/mL, from about 28 μg/mL to about 48 μg/mL, from about 30 μg/mL to about 50 μg/mL, from about 35 μg/mL to about 55 μg/mL, from about 40 μg/mL to about 60 μg/mL, from about 45 μg/mL to about 65 μg/mL, from about 50 μg/mL to about 75 μg/mL, from about 60 μg/mL to about 240 μg/mL, from about 70 μg/mL to about 350 μg/mL, from about 80 μg/mL to about 400 μg/mL, from about 90 μg/mL to about 450 μg/mL, from about 100 μg/mL to about 500 μg/mL, from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 36 mg/mL, from about 18 mg/mL to about 38 mg/mL, from about 20 mg/mL to about 40 mg/mL, from about 22 mg/mL to about 42 mg/mL, from about 24 mg/mL to about 44 mg/mL, from about 26 mg/mL to about 46 mg/mL, from about 28 mg/mL to about 48 mg/mL, from about 30 mg/mL to about 50 mg/mL, from about 35 mg/mL to about 55 mg/mL, from about 40 mg/mL to about 60 mg/mL, from about 45 mg/mL to about 65 mg/mL, from about 50 mg/mL to about 75 mg/mL, from about 60 mg/mL to about 240 mg/mL, from about 70 mg/mL to about 350 mg/mL, from about 80 mg/mL to about 400 mg/mL, from about 90 mg/mL to about 450 mg/mL, from about 100 mg/mL to about 500 mg/mL, from about 0.01 g/mL to about 1 g/mL, from about 0.05 g/mL to about 2 g/mL, from about 1 g/mL to about 5 g/mL, from about 2 g/mL to about 10 g/mL, from about 4 g/mL to about 16 g/mL, or from about 5 g/mL to about 20 g/mL.

### Gels and Hydrogels

In some embodiments, SBPs are or are combined with gels or hydrogels. As used herein, the term “gel” refers to a dispersion of liquid molecules in a solid medium. Gels in which the dispersed liquid molecules include water are referred to herein as “hydrogels.” Gels in which the dispersed liquid molecules include an organic phase are referred to herein as “organogels.” The solid medium may include polymer networks.

In some embodiments, SBP gels or hydrogels are prepared with processed silk. In processed silk gels, polymer networks may include silk fibroin. In some embodiments, gels are prepared with one or more therapeutic agents. In some embodiments, gels include one or more excipients. The excipients may be selected from any of those described herein. In some embodiments, excipients may include salts. In some embodiments, the excipients may include gelling agents. In some embodiments, gels are prepared with one or more therapeutic agents, biological agents, proteins, small molecules, and/or polymers.

Gel preparation may require varying temperatures and incubation times for gel polymer networks to form. In some embodiments, processed silk preparations are heated to 37° C. to prepare gels. In some embodiments, processed silk preparations are incubated for from about 2 hours to about 36 hours or more to promote gel formation. In some embodiments, gel formation requires mixing with one or more gelling agents or excipients. Mixing may be carried out under various temperatures and lengths of time to allow gel polymer networks to form. Gel formation may require homogenous dispersion of gelling agents or excipients. In some embodiments, processed silk preparations used to prepare gels include silk fibroin. Gel formation for processed silk gels may require incubation at 37° C. for up to 24 hours. Some gels may be stored for later use or processing. In some embodiments, gels are stored at 4° C.

In some embodiments, processed silk gels include excipient or gelling agent at a concentration of from about 0.01% to about 0.1%, from about 0.1% (w/v) to about 1% (w/v), from about 0.5% (w/v) to about 5% (w/v), from about 1% (w/v) to about 10% (w/v), from about 5% (w/v) to about 15% (w/v), from about 10% (w/v) to about 30% (w/v), from about 15% (w/v) to about 45% (w/v), from about 20% (w/v) to about 55% (w/v), from about 25% (w/v) to about 65% (w/v), from about 30% (w/v) to about 70% (w/v), from about 35% (w/v) to about 75% (w/v), from about 40% (w/v) to about 80% (w/v), from about 50% (w/v) to about 85% (w/v), from about 60% (w/v) to about 90% (w/v), from about 75% (w/v) to about 95% (w/v), from about 90% (w/v) to about 96% (w/v), from about 92% (w/v) to about 98% (w/v), from about 95% (w/v) to about 99% (w/v), from about 98% (w/v) to about 99.5% (w/v), or from about 99% (w/v) to about 99.9% (w/v).

In some embodiments, processed silk gels (e.g., hydrogels or organogels) include silk fibroin at a concentration of from about 0.01% to about 0.1%, from about 0.1% (w/v) to about 1% (w/v), from about 0.5% (w/v) to about 5% (w/v), from about 1% (w/v) to about 10% (w/v), from about 5% (w/v) to about 15% (w/v), from about 10% (w/v) to about 30% (w/v), from about 15% (w/v) to about 45% (w/v), from about 20%4 (w/v) to about 55% (w/v), from about 25% (w/v) to about 65% (w/v), from about 30% (w/v) to about 70% (w/v), from about 35% (w/v) to about 75% (w/v), from about 40% (w/v) to about 80% (w/v), from about 50% (w/v) to about 85% (w/v), from about 60% (w/v) to about 90% (w/v), from about 75% (w/v) to about 95% (w/v), from about 90% (w/v) to about 96% (w/v), from about 92% (w/v) to about 98% (w/v), from about 95% (w/v) to about 99% (w/v), from about 98% (w/v) to about 99.5% (w/v), or from about 99% (w/v) to about 99.9% (w/v). Silk fibroin included may be from a silk fibroin preparation with an average silk fibroin molecular weight or range of molecular weights of from about 3.5 kDa to about 10 kDa, from about 5 kDa to about 20 kDa, from about 10 kDa to about 30 kDa, from about 15 kDa to about 40 kDa, from about 20 kDa to about 50 kDa, from about 25 kDa to about 60 kDa, from about 30 kDa to about 70 kDa, from about 35 kDa to about 80 kDa, from about 40 kDa to about 90 kDa, from about 45 kDa to about 100 kDa, from about 50 kDa to about 110 kDa, from about 55 kDa to about 120 kDa, from about 60 kDa to about 130 kDa, from about 65 kDa to about 140 kDa, from about 70 kDa to about 150 kDa, from about 75 kDa to about 160 kDa, from about 80 kDa to about 170 kDa, from about 85 kDa to about 180 kDa, from about 90 kDa to about 190 kDa, from about 95 kDa to about 200 kDa, from about 100 kDa to about 210 kDa, from about 115 kDa to about 220 kDa, from about 125 kDa to about 240 kDa, from about 135 kDa to about 260 kDa, from about 145 kDa to about 280 kDa, from about 155 kDa to about 300 kDa, from about 165 kDa to about 320 kDa, from about 175 kDa to about 340 kDa, from about 185 kDa to about 360 kDa, from about 195 kDa to about 380 kDa, from about 205 kDa to about 400 kDa, from about 215 kDa to about 420 kDa, from about 225 kDa to about 440 kDa, from about 235 kDa to about 460 kDa, or from about 245 kDa to about 500 kDa.

Gelling agents may be used to facilitate sol-gel transition. As used herein, the term “sol-gel transition” refers to the shift of a formulation from a solution to a gel. In some embodiments, the use of gelling agents may be carried out according to any of such methods described in International Publication No. WO2017139684, the contents of which are herein incorporated by reference in their entirety. Gelling agents may be water-soluble, waxy solids. In some embodiments, gelling agents may be water-soluble and hygroscopic in nature. In some embodiments, gelling agents may include polar molecules. Gelling agents may have net positive, net negative, or net neutral charges at a physiological pH (e.g., pH of about 7.4). Some gelling agents may be amphipathic. Additional examples of gelling agents include oils (e.g., castor, corn oil, cottonseed oil, olive oil, peanut oil, peppermint oil, safflower oil, sesame oil, soybean oil, hydrogenated vegetable oil, hydrogenated soybean oil, and medium-chain triglycerides of coconut oil and/or palm seed oil), emulsifiers [e.g., polyoxyl 40 stearate (PEG 1750 monosterate), polyoxyl 8 stearate (PEG 400 monosterate), polysorbate 20, polysorbate-SO, or poloxamer], surfactants (e.g., polysorbate, poloxamer, sodium dodecyl sulfate, Triton X100, or tyloxapol), and suspending agents (e.g., polyvinyl pyrrolidone, polyvinyl pyrrolidone-12, polyvinyl pyrrolidone-17, hydroxyethyl cellulose, or carboxymethyl cellulose).

In some embodiments, gel formation is induced by applying one or more of the following to processed silk preparations: ultrasound, sonication, shear forces, temperature change (e.g., heating), addition of precipitants, modulation of pH, changes in salt concentration, chemical cross-linking, chemical modification, seeding with preformed hydrogels, increasing silk fibroin concentration, modulating osmolarity, use of electric fields, or exposure to electric currents. In some embodiments, methods of inducing gel formation may include, but are not limited to any of those described in International Publication No. WO2005012606 or United States Publication No. US2011/0171239, the contents of each of which are herein incorporated by reference in their entirety.

In some embodiments, processed silk gel preparation may be carried with the aid of sonication. As used herein, the term “sonication” refers to a process of agitation using sound energy. Sonication conducted at frequencies greater than 20 kHz is referred to as ultrasonication. Sonication may aid in gel formation by dispersing and/or agitating polymer components within a solution to foster an arrangement that favors polymer network formation. The polymer network may include silk fibroin. In some embodiments, the use of sonication for gel preparation may be carried out according to any of the methods described in Zhao et al. (2017) Materials Letters 211:110-113 or Mao et al. (2017) Colloids Surf B Biointerfaces 160:704-714), the contents of each of which are herein incorporated by reference in their entirety.

In some embodiments, processed silk gel formation may be carried out using shear forces. As used herein, the term “shear forces” refers to unaligned forces that apply pressure to two or more different parts of an object or medium from different or opposing directions. Shear forces are distinct from compression forces, which are directed toward each other. Shear forces may be applied during processed silk gel preparation using a syringe, tubing, needle, or other apparatus capable of increasing shear forces. Processed silk preparation may be pushed through a syringe, tubing, needle, or other apparatus to generate shear forces. The use of shear forces in gel formation may include any of those described in United States Publication No. US2011/0171239, the contents of which are herein incorporated by reference in their entirety.

In some embodiments, changes in temperature may be used to aid in processed silk gel formation. Changes in temperature may be used to disperse or align polymer components in an arrangement that promotes gel polymer network formation. The polymer components may include silk fibroin. In some embodiments, gel formation may be carried out by raising or lowering the temperature of a processed silk preparation to from about 0° C. to about 5° C., from about 2° C. to about 6C, from about 4° C. to about 12° C., from about 8° C. to about 16° C., from about 10° C. to about 26° C., from about 15° C. to about 28° C., from about 20° C. to about 32° C., from about 25° C. to about 34° C., from about 30° C. to about 45° C., from about 35° C. to about 55° C., from about 37° C. to about 65° C., from about 40° C. to about 75° C., from about 50° C. to about 100° C., from about 60° C. to about 120° C., from about 70° C. to about 140° C., from about 80° C. to about 160° C., or from about 100° C. to about 300° C. In some embodiments, one or more excipients or gelling agents may be included to lower the temperature necessary for gel formation to occur. Such embodiments may be employed to protect temperature-sensitive components embedded within gels. In some embodiments, gel formation is carried out at 4° C. Glycerol, polyethylene glycol (PEG), and/or polymers of PEG (e.g., PEG400) may be included in processed silk preparations as excipients to lower the temperature necessary to form a gel. The gel may be a silk fibroin gel. Excipient concentration may be about 30% (w/v). Silk fibroin concentration may be from about 2% to about 30%.

In some embodiments, gel formation is carried out by applying an electric current, also referred to as “electrogelation.” Electrogelation may be carried out according to any of the methods presented in International Publication No. WO2010036992, the contents of which are herein incorporated by reference in their entirety. In some embodiments, a reverse voltage may be applied to reverse gel formation and regenerate a processed silk solution.

In some embodiments, gel formation is carried out by modulating the pH of processed silk preparations. Gel formation through pH modulation may be carried out according to the methods described in International Publication No. WO2005012606, United States Publication No. US2011/0171239, and Dubey et al. (2017) Materials Chemistry and Physics 203:9-16, the contents of each of which are herein incorporated by reference in their entirety.

In some embodiments, gel formation is carried out in association with modulating the osmolarity of a processed silk preparation. As used herein, the term “osmolarity” or “osmotic concentration” refers to the number of osmoles of solute in solution on a per liter basis (Osm/L). Unlike molarity, which is a measure of the number of moles solute per liter of solvent (M), osmolarity factors in the effect of ions on osmotic pressure. For example, a 1 M solution of NaCl would have an osmolarity of 2 Osm/L while a 1 M solution of MgCl₂ would have an osmolarity of 3 Osm/L. Hypo- or hyper-osmotic formulations can lead to local tissue damage and reduced biocompatibility. In some embodiments, the osmolarity of processed silk gels is modulated by controlling the type, molecular weight, and/or concentration of excipients included. Osmolarity may be modulated by varying the concentration and/or molecular weight of salts used in processed silk preparations. In some embodiments, osmolarity is reduced by using lower molecular weight gelling agents. For example, 4 kDa PEG may be used in place of PEG400. The use of Poloxamer 188 at 10% (w/v) may reduce osmolarity in comparison to lower molecular weight species such as glycerol. In some embodiments, sodium chloride may be added to increase osmolarity. In some embodiments, osmolarity is adjusted to fall between 280 and 320 mOsm/L.

In some embodiments, gel formation is carried out through seeding. As used herein when referring to gel formation, “seeding” refers to a process of inducing gel formation using a small amount of pre-formed gel. Seeding may promote gel formation by encouraging polymer network formation to build off of the pre-formed gel introduced. In some embodiments the gel includes silk fibroin. Seeding with a pre-formed silk fibroin hydrogel may be used to promote transition of a silk fibroin solution into a silk fibroin gel. In some embodiments, seeding reduces the need for gelling agents and/or excipients to form gels.

In some embodiments, gel formation is carried out using chemical cross-linking. As used herein, the term “chemical cross-linking” refers to a process of forming covalent bonds between chemical groups from different molecules or between chemical groups present on different parts of the same molecule. In some embodiments, chemical cross-linking may be carried out by contacting processed silk preparations with ethanol. Such methods may be carried out according to those described in Shi et al. (2017) Advanced Material 29(29):1701089, the contents of which are herein incorporated by reference in their entirety. In some embodiments, cross-linking may be carried out using enzymes. Methods of enzyme cross-linking using horse radish peroxidase may include any of those described in McGill et a (2017) Acta Biomaterialia 63:76-84 or Guo et al. (2017) Biomaterials 145:44-55, the contents of each of which are herein incorporated by reference in their entirety. In some embodiments, chemical cross-linking may be photo-initiated, as disclosed in International Publication No. WO2017123383 and in Zhang et al. (2017) Fibers and Polymers 18(10):1831-1840, the contents of each of which are herein incorporated by reference in their entirety.

In some embodiments, other chemical modifications may be used during processed silk gel preparation. Some chemical modifications may be used to induce silk fibroin 0-sheet conformations. In some embodiments, this process involves contact with a chemical. Chemicals may include, but are not limited to, ethanol. In some embodiments, silk fibroin may be chemically crosslinked with other materials during gel preparation. Such materials may include other peptides (e.g., see Guo et al (2017) Biomaterials 145:44-55, the contents of which are herein incorporated by reference in their entirety). In some embodiments, processed silk gels are prepared by formation of internal chemical cross-links. These crosslinks may be dityrosine crosslinks (e.g., see International Publication No. WO2017123383, the contents of which are herein incorporated by reference in their entirety). In some embodiments, photosensitive materials may be used to promote chemical modifications. Such materials may include riboflavin (e.g., see International Publication No. WO2017123383). In some embodiments, processed silk gels may be functionalized with particles. These particles may be microspheres and/or nanospheres (e.g., see Ciocci et al. (2017) Int J Biol Macromol S0141-8130(17):32839-8, the contents of which are herein incorporated by reference in their entirety).

In some embodiments, SBP gels or hydrogels may shear thin or display shear thinning properties.

### Particles

In some embodiments, SBPs are particles. As used herein, the term “particle” refers to a minute portion of a substance. SBP particles may include particles of processed silk. Processed silk particles may include silk fibroin particles. Silk fibroin particles may be tiny clusters of silk fibroin or they may be arranged as more ordered structures. Particles may vary in size. Processed silk particles may be visible or may be too tiny to view easily with the naked eye. Particles with a width of from about 0.1 μm to about 100 μm are referred to herein as “microparticles.” Particles with a width of about 100 nm or less are referred to herein as “nanoparticles.” Microparticles and nanoparticles that are spherical in shape are termed microspheres and nanospheres, respectively. Processed silk particle preparations may include particles with uniform width or with ranges of widths. In some embodiments, processed silk particle preparations include average particle widths of or ranges of particle widths of from about 10 nm to about 25 nm, from about 20 nm to about 50 nm, from about 30 nm to about 75 nm, from about 40 nm to about 80 nm, from about 50 nm to about 100 nm, from about 0.05 μm to about 10 μm, from about 1 μm to about 20 μm, from about 2 μm to about 30 μm, from about 5 μm to about 40 μm, from about 10 μm to about 50 μm, from about 20 μm to about 60 μm, from about 30 μm to about 70 μm, from about 40 μm to about 80 μm from about 50 μm to about 90 μm, from about 0.05 mm to about 2 mm, from about 0.1 mm to about 3 mm, from about 0.2 mm to about 4 mm, from about 0.5 mm to about 5 mm, from about 1 mm to about 6 mm, from about 2 mm to about 7 mm, from about 5 mm to about 10 mm, from about 10 nm to about 100 μm, from about 10 μm to about 10 mm, from about 50 nm to about 500 μm, from about 50 μm to about 5 mm, from about 100 nm to about 10 mm, or from about 1 μm to about 10 mm. In some embodiments, processed silk particle preparations include average particle widths of at least 10 nm, at least 100 nm, at least 0.5 μm, at least 1 μm, at least 10 μm, at least 100 μm, at least 500 μm, at least 1 mm, or at least 10 mm.

Processed silk particles may be formed through spraying of a processed silk preparation. In some embodiments, electrospraying is used. Electrospraying may be carried out using a coaxial electrospray apparatus (e.g., see Cao et al. (2017) Scientific Reports 7:11913, the contents of which are herein incorporated by reference in their entirety). In some embodiments, silk fibroin microspheres or nanospheres may be obtained by electrospraying a silk fibroin preparation into a collector and flash freezing the sprayed particles (e.g., see United States Publication No. US2017/0333351, the contents of which are herein incorporated by reference in their entirety). The flash frozen silk fibroin particles may then be lyophilized. In some embodiments, processed silk particles may be prepared using centrifugal washing, followed by lyophilization, as taught in United States Publication No. US2017/0340575, the contents of which are herein incorporated by reference in their entirety. In some embodiments, processed silk microspheres may be formed through the use of a microfluidic device (e.g., see Sun et al. (2017) Journal of Materials Chemistry B 5:8770-8779, the contents of which are herein incorporated by reference in their entirety). In some embodiments, microspheres are formed via coagulation in a methanol bath, as taught in European Patent No. EP3242967, the contents of which are herein incorporated by reference in their entirety.

### Scaffolds

In some embodiments, SBPs include scaffolds. As used herein, a “scaffold” refers to a framework used for support. SBP scaffolds may include scaffolds formed using processed silk frameworks. Processed silk may include a polymeric network that provides a framework to support a variety of materials related to a variety of applications. Such application may include, but are not limited to, biological, material, cosmetic, veterinary, agricultural, and therapeutic applications. In some embodiments, processed silk scaffolds include polymeric networks that include silk fibroin. In some embodiments, processed silk scaffolds include one or more of silk fibers, nanofibers, mats, films, foams, membranes, rods, tubes, gels, hydrogels, microspheres, nanospheres, solutions, patches, grafts, and powders. In some embodiments, processed silk scaffolds include other agents. Such agents may include, but are not limited to, polymers, synthetic polymers, small molecules, therapeutics, proteins, peptides, hormones, enzymes, drugs, oxidants, antioxidants, macromolecules, microspheres, nanospheres, antibodies, cells, tissues, organs, organisms, decellularized pulp, nucleic acids, DNA, RNA, known drugs, NSAIDS, hydrophobic agents, hydrophilic agents, vitamins, minerals, ions, metals, carbohydrates, fats, polycaprolactone, nano-hydroxyapatite, polyurethane, bacterial cellulose, chitosan, steroids, lipids, ionic liquids, nanoparticles, particles, curcumin, salts, polyethylene, ultra-high-molecular weight polyethylene, VEGF, gelatin, PEG, and polyethylene oxide.

In some embodiments, processed silk scaffolds are prepared by casting a processed silk preparation into a mold, and allowing the preparation to solidify to obtain the desired shape. Any mold shape may be used. In some embodiments, injection molding machines are used. Molding may be performed at various temperatures needed to facilitate filling of molds and solidification into final molded form. In some embodiments, molding is performed at room temperature. In other embodiments, the molding is performed at 160° C. In some embodiments, molding is carried out according to the methods described in International Publication No. WO2017179069, Thai et al. J Biomed Mater (2017) 13(1):015009, or Chen et al. (2017) PLoS One 12(11): e0187880, the contents of each of which are herein incorporated by reference in their entirety.

In some embodiments, processed silk scaffolds are prepared by coating a scaffold formed from non-silk materials with a processed silk preparation. The processed silk may include silk fibroin. The non-silk materials may include, but are not limited to, natural or synthetic polymers, fibers, nanofibers, mats, films, foams, membranes, rods, tubes, gels, hydrogels, microspheres, nanospheres, nanoparticles, particles, solutions, patches, and/or grafts. Methods of coating a scaffold with a processed silk preparation are taught in Ai et at (2017) International Journal of Nanomedicine 12:7737-7750 and Jiang et al. (2017) J Biomater Sci Polym Ed 15:1-36, the contents of each of which are herein incorporated by reference in their entirety.

In some embodiments, processed silk scaffolds are prepared using three-dimensional (3D) printing. 3D printing may be carried out using a processed silk preparation to form the scaffold. In some embodiments, a scaffold is 3D printed from other materials, then modified with processed silk preparation (e.g., coated with processed silk). In some embodiments, SBPs may be prepared and used as an ink during the 3D printing process. The 3D printed scaffolds may be further modified after their fabrication. Methods of 3D printing processed silk scaffolds may be carried out according to any of those taught in Costa et al. (2017) Adv Healthc Mater 1701021, the contents of which are herein incorporated by reference in their entirety.

In some embodiments, processed silk scaffolds are prepared via salt leaching. As used herein, the term “salt leaching” refers to a process whereby a polymer is poured over salt crystals and allowed to polymerize before the salt crystals are dissolved with solvent to yield a porous scaffold. Processed silk preparations may be used as the polymer in such methods. The processed silk may include silk fibroin. The salt used may be monovalent or divalent. Examples of salts include, but are not limited to, NaCl, CaCl₂, KCl, NaBr, KFI, MgSO₄, and MgCl₂. In some embodiments, scaffold preparation by salt leaching may be carried out according to the methods presented in International Publication No. WO2005012606, the contents of which are herein incorporated by reference in their entirety.

### Devices

In some embodiments, SBPs may be devices or may be included as device components. As used herein, the term “device” refers to any article constructed or modified to suit a particular purpose. Devices may be designed for a variety of purposes, including, but not limited to, therapeutic applications, material science applications, and agricultural applications. In some embodiments, SBPs are embedded or incorporated into devices. Some devices include SBPs as coatings or lubricants. In some embodiments, devices include implants, patches, mesh, sponges, grafts, insulators, pipes, prosthetics, resistors, bedding, blankets, liners, ropes, plugs, fillers, electronic devices, mechanical devices, medical devices, surgical devices, veterinary devices, and agricultural devices. Additional devices are described herein.

### II. Therapeutic Applications

In some embodiments, SBPs may be used in a variety of therapeutic applications. As used herein, the term “therapeutic application” refers to any method related to restoring or promoting the health, nutrition, and/or wellbeing of a subject; supporting or promoting reproduction in a subject; or treating, preventing, mitigating, alleviating, curing, or diagnosing a disease, disorder, or condition. As used herein, the term “condition” refers to a physical state of wellbeing. Therapeutic applications may include, but are not limited to, medical applications, surgical applications, and veterinary applications. As used herein, the term “medical application” refers to any method or use that involves treating, diagnosing, and/or preventing disease according to the science of medicine. “Surgical applications” refer to methods of treatment and/or diagnosis that involve operation on a subject, typically requiring incision and the use of instruments. “Veterinary applications” refer to therapeutic applications where the subject is a non-human animal. In some embodiments, therapeutic applications may include, but are not limited to, experimental, diagnostic, or prophylactic applications. In some embodiments, therapeutic applications include preparation and/or use of therapeutic devices. As used herein, the term “therapeutic device” refers to any article prepared or modified for therapeutic use.

SBPs used for therapeutic applications may include or may be combined with one or more pharmaceutical compositions, implants, therapeutic agents, coatings, foods, health supplements, excipients, or devices. In some embodiments, SBPs facilitate the delivery and/or controlled release of therapeutic agent payloads. In some embodiments, SBPs described herein may be used in gene therapy and/or gene editing. In some embodiments, SBPs described herein may be used in immunotherapy. Some SBPs may be used for diagnostic applications, in in vitro cell culture, tissue engineering, and/or surgery. In some embodiments, SBPs described herein may be used to stabilize therapeutic agents. Some SBPs may be used as tools, materials, or devices in therapeutic applications. Such SBPs may include, but are not limited to, delivery vehicles, scaffolds, structural supports, and sutures.

### Subjects

Therapeutic applications of the present disclosure may be applied to a variety of subjects. As used herein, the term “subject” refers to any entity to which a particular processor activity relates to or is applied. Non-limiting examples of subjects are presented in Table 2. Subjects of therapeutic applications described herein may be human or non-human. Human subjects may include humans of different ages, genders, races, nationalities, or health status. Non-human subjects may include non-human animal subjects (also simply referred to herein as “animal subjects”). Animal subjects may be non-human vertebrates or invertebrates. Some animal subjects may be wild type or genetically modified organisms (e.g. transgenic). In some embodiments, subjects include patients. As used herein, the term “patient” refers to a subject seeking treatment, in need of treatment, requiring treatment, receiving treatment, expecting treatment, or who is under the care of a trained (e.g., licensed) professional for a particular disease, disorder, and/or condition.

TABLE 2
Subjects

| Subject | Category |
|---|---|
| human | human |
| alpaca | non-human |
| ape | non-human |
| baboon | non-human |
| banteng | non-human |
| bass | non-human |
| bison | non-human |
| bonobo | non-human |
| caecilian | non-human |
| caique | non-human |
| camel | non-human |
| canary | non-human |
| carp | non-human |
| cat | non-human |
| catfish | non-human |
| cattle | non-human |
| cattle egret | non-human |
| chicken | non-human |
| chimpanzee | non-human |
| cockatiel | non-human |
| cockatoo | non-human |
| conure | non-human |
| deer | non-human |
| dog | non-human |
| donkey | non-human |
| dove | non-human |
| duck | non-human |
| eel | non-human |
| elk | non-human |
| finch | non-human |
| frog | non-human |
| gayal | non-human |
| geese | non-human |
| goat | non-human |
| gorilla | non-human |
| guinea pig | non-human |
| halibut | non-human |
| horse | non-human |
| iguana | non-human |
| lizard | non-human |
| llama | non-human |
| lovebird | non-human |
| macaque | non-human |
| macaw | non-human |
| monkey | non-human |
| mouse | non-human |
| mule | non-human |
| mullet | non-human |
| parakeet | non-human |
| parrot | non-human |
| parrotlet | non-human |
| pig | non-human |
| pigeon | non-human |
| pionus | non-human |
| primate | non-human |
| quail | non-human |
| rabbit | non-human |
| rat | non-human |
| reindeer | non-human |
| rosella | non-human |
| salmon | non-human |
| shark | non-human |
| sheep | non-human |
| snake | non-human |
| sturgeon | non-human |
| tortoise | non-human |
| trout | non-human |
| tuna | non-human |
| turkey | non-human |
| turtle | non-human |
| water buffalo | non-human |
| yak | non-human |
| abalone | non-human, invertebrate |
| arachnid | non-human, invertebrate |
| clam | non-human, invertebrate |
| crab | non-human, invertebrate |
| crayfish | non-human, invertebrate |
| cricket | non-human, invertebrate |
| honey bee | non-human, invertebrate |
| insect | non-human, invertebrate |
| lobster | non-human, invertebrate |
| moth | non-human, invertebrate |
| mussel | non-human, invertebrate |
| oyster | non-human, invertebrate |
| prawn | non-human, invertebrate |
| scallop | non-human, invertebrate |
| silk moth | non-human. invertebrate |
| shrimp | non-human, invertebrate |
| silk worm | non-human, invertebrate |
| spider | non-human, invertebrate |

### Veterinary Applications

In some embodiments, SBPs may be used in veterinary applications to restore or promote the health and/or wellbeing of a non-human animal subject and/or to treat, prevent, alleviate, cure, or diagnose a disease, disorder, or condition of a non-human animal subject. In some embodiments. SBPs of the present disclosure may be used to improve animal health, nutrition, performance (e.g., performance of show animals or farm animals), fertility, milk production, egg production, or fur production. The pharmacokinetics and efficacy studies of SBPs for veterinary applications may be analyzed via any method known to one skilled in the art. As a non-limiting example, the SBPs may be used for companion animal health. As another non-limiting example, the SBPs may be used for farm animal health.

In some embodiments, SBPs of the present disclosure may be used to improve the performance of a show animal. A show animal is a domestic animal breed for either physical, mental, or appearance competitions. These competitions may include, but are not limited to racing, tests of agility, tests of strength, and shows (e.g. dog shows). In some embodiments SBPs may be used to enhance the shelf life and stability of items used for performance enhancement. Non-limiting examples of items used for performance enhancement are food, nutritional supplements, nutrients, vitamins, minerals, antibiotics, health supplements, produce supplements, dietary supplements, pastes, nasal strips, blankets, housing, bedding, clothing, footwear (e.g. horseshoes), feeding equipment (e.g. bowls and water bottles), brushes, bandages, barns, coops, cages, stalls, liners, enclosures, ropes, ties, pens, flooring, shelters, ventilations systems, and wires and hormone supplements. In some embodiments, SBPs described herein may be used to deliver a payload and/or therapeutic agent to improve the performance of show animals. Non-limiting examples of payloads and/or therapeutic agents are antibiotics, drugs, small molecules, proteins, nutrients, vitamins, minerals, health supplements, produce supplements, and chemicals.

In some embodiments, SBPs may be used to improve animal feed. Such SBPs may be used to enhance the stability and/or shelf life of animal feed (e.g., see improvements to human food described in Marelli et al. (2016) Scientific Reports 6:25263, the contents of which are herein incorporated by reference in their entirety). In some embodiments, SBPs may be provided as animal feed. Such SBPs may improve animal health through nutritional or other therapeutic properties. In some embodiments, SBPs may be used to administer health supplements, produce supplements, hormone supplements, nutrients, vitamins, therapeutic agents, antibiotics, and/or birth control through animal feed. Such methods may include any of those described in International Publication Number WO2017142906 or U.S. Pat. No. 8,778,385, the contents of each of which are herein incorporated by reference in their entirety. In some embodiments, SBP animal feed may be used to increase production of products obtained though animal husbandry.

In some embodiments, SBPs of the present disclosure may be used for pain treatment in a non-human animal. For example, many pets, zoo animals, or farm animals need post-operative pain management after a surgical or dental procedure. In some embodiments, SBPs may be used for post-operative pain treatment in a feline. The feline may be a cat, a cheetah, a puma, a jaguar, a leopard, a lion, a lynx, a tiger, or the like. In some embodiments, SBPs may be used for post-operative pain treatment in a canine. The canine may be a dog, a wolf, a coyote, a fox, a jackal, a dingo, or the like. In some embodiments, SBPs may be used for treating osteoarthritic pain in dogs. In some embodiments, SBPs may include analgesic agents (e.g., any of those described herein) as cargo or payloads for treatment of pain in these animals. In some embodiments, the analgesic agents incorporated into the SBPs may include an opioid analgesic (e.g., morphine, codeine, fentanyl, buprenorphine, and hydromorphone), a corticosteroid (e.g., cortisone, prednisone, prednisolone, methylprednisolone, and dexamethasone), other analgesics (e.g., gabapentin and amitriptyline), and/or any combination thereof. In some embodiments, an opioid analgesic, such as buprenorphine, is loaded into SBP gels or hydrogels for extended release (e.g., 3-5 days) in a non-human animal.

In some embodiments, SBPs of the present disclosure may be used for treating dry eye disease in a non-human animal. In one embodiment, SBPs are used for treating dry eye disease in dogs.

In some embodiments, SBPs of the present disclosure may be used for dental treatments in a non-human animal.

In some embodiments, SBPs of the present disclosure may be used for orthopedic treatments in a non-human animal.

### Therapeutic Agents

In some embodiments, therapeutic applications involve the use of SBPs that are therapeutic agents or are combined with one or more therapeutic agents. As used herein, the term “therapeutic agent” refers to any substance used to restore or promote the health and/or wellbeing of a subject and/or to treat, prevent, alleviate, cure, or diagnose a disease, disorder, or condition. Examples of therapeutic agents include, but are not limited to, adjuvants, analgesic agents, antiallergic agents, antiangiogenic agents, antiarrhythmic agents, antibacterial agents, antibiotics, antibodies, anticancer agents, anticoagulants, antidementia agents, antidepressants, antidiabetic agents, antigens, antihypertensive agents, anti-infective agents, anti-inflammatory agents, antioxidants, antipyretic agents, anti-rejection agents, antiseptic agents, antitumor agents, antiulcer agents, antiviral agents, biological agents, birth control medication, carbohydrates, cardiotonics, cells, chemotherapeutic agents, cholesterol lowering agents, cytokines, endostatins, enzymes, fats, fatty acids, genetically engineered proteins, glycoproteins, growth factors, health supplements, hematopoietics, herbal preparations, hormones, hypotensive diuretics, immunological agents, inorganic synthetic pharmaceutical drugs, ions, lipoproteins, metals, minerals, nanoparticles, naturally derived proteins, NSAIDs, nucleic acids, nucleotides, organic synthetic pharmaceutical drugs, oxidants, peptides, pills, polysaccharides, proteins, protein-small molecule conjugates or complexes, psychotropic agents, small molecules, sodium channel blockers, statins, steroids, stimulants, therapeutic agents for osteoporosis, therapeutic combinations, thrombopoietics, tranquilizers, vaccines, vasodilators, VEGF-related agents, veterinary agents, viruses, virus particles, and vitamins. In some embodiments, SBP therapeutics and methods of delivery may include any of those taught in International Publication Numbers WO2017139684, WO2010123945, WO2017123383, or United States Publication Numbers US20170340575, US20170368236, and US20110171239 the contents of each of which are herein incorporated by reference in their entirety. In some embodiments, therapeutic agents may be selected from any of those listed in Table 3. In the Table, example categories are indicated for each therapeutic agent. These categories are not limiting and each therapeutic agent may fall under multiple categories (e.g., any of the categories of therapeutic agents described herein).

TABLE 3
Therapeutic agents

| Agent | Category |
|---|---|
| opium | analgesic agent |
| opiate | analgesic agent |
| doxycycline monohydrate | antibacterial agent |
| tigecycline | antibacterial agent |
| doxycycline hyclate | antibacterial agent |
| vibramycin | antibacterial agent |
| doxycycline hydrochloride hemiethanolate hemihydrate | antibacterial agent |
| doxycycline calcium | antibacterial agent |
| abciximab | antibody |
| adalimumab | antibody |
| adalimumab-atto | antibody |
| alefacept | antibody |
| alemtuzumab | antibody |
| antibody fragment | antibody |
| antibody-drug conjugate | antibody |
| atezolizumab | antibody |
| basiliximab | antibody |
| belimumab | antibody |
| bezlotoxumab | antibody |
| bivalent antibody | antibody |
| canakinumab | antibody |
| certolizumab pegol | antibody |
| cetuximab | antibody |
| daclizumab | antibody |
| denosumab | antibody |
| efalizumab | antibody |
| golimumab | antibody |
| inflectra | antibody |
| ipilimumab | antibody |
| ixekizumab | antibody |
| monoclonal antibody | antibody |
| monovalent antibody | antibody |
| multivalent antibody | antibody |
| natalizumab | antibody |
| nivolumab | antibody |
| obiltoxaxamab | antibody |
| olaratumab | antibody |
| omalizumab | antibody |
| palivizumab | antibody |
| panitumumab | antibody |
| pembrolizumab | antibody |
| polyclonal antibody | antibody |
| reslizumab | antibody |
| rituximab | antibody |
| secukinumab | antibody |
| tocilizumab | antibody |
| trastuzumab | antibody |
| ustekinumab | antibody |
| autoantigen | antigen |
| endogenous antigen | antigen |
| exogenous antigen | antigen |
| neoantigen | antigen |
| tumor antigen | antigen |
| viral antigen | antigen |
| exogenous antigen | antigen |
| endogenous antigen | antigen |
| autoantigen | antigen |
| neoantigen | antigen |
| viral antigen | antigen |
| tumor antigen | antigen |
| xenogenus (heterologous) antigen | antigen |
| autologous antigen | antigen |
| idiotypic antigen | antigen |
| allogenic (homologous) antigen | antigen |
| epitope | antigen |
| tumor-specific antigen | antigen |
| tumor-associated antigen | antigen |
| neo-epitope | antigen |
| allergen | antigen |
| superantigen | antigen |
| tolerogen | antigen |
| immunoglobulin-binding protein | antigen |
| t-dependent antigen | antigen |
| t-independent antigen | antigen |
| immunodominant antigen | antigen |
| COX-1 inhibitor | anti-inflammatory agent |
| COX-2 inhibitor | anti-inflammatory agent |
| bisbiguanides polymeric quaternary | antiseptic agent |
| ammonium compound | |
| chlorhexidine | antiseptic agent |
| chlorinated phenol | antiseptic agent |
| ethanol | antiseptic agent |
| hydrogen peroxide | antiseptic agent |
| lower alcohol | antiseptic agent |
| peroxides | antiseptic agent |
| propanol | antiseptic agent |
| quaternary amine surfactant | antiseptic agent |
| silver complex | antiseptic agent |
| small molecule quaternary ammonium | antiseptic agent |
| compound | |
| 5-FU Enhancer | anticancer agent |
| 9-AC | anticancer agent |
| abraxane | anticancer agent |
| actinomycin | anticancer agent |
| AG2037 | anticancer agent |
| AG3340 | anticancer agent |
| Aggrecanase Inhibitor | anticancer agent |
| alitretinoin | anticancer agent |
| alkylating agent | anticancer agent |
| Aminoglutethimide | anticancer agent |
| Amsacrine (m-AMSA) | anticancer agent |
| anthracycline | anticancer agent |
| antimicrobial peptide | anticancer agent |
| Asparaginase | anticancer agent |
| Azacitidine | anticancer agent |
| azathioprine | anticancer agent |
| Batimastat (BB94) | anticancer agent |
| BAY 12-9566 | anticancer agent |
| BCH-4556 | anticancer agent |
| bexarotene | anticancer agent |
| Bis-Naphtalimide | anticancer agent |
| bleomycin | anticancer agent |
| Busulfan | anticancer agent |
| Capecitabine | anticancer agent |
| Carboplatin | anticancer agent |
| Cannustaine + Polifepr Osan | anticancer agent |
| cdk4/cdk2 inhibitor | anticancer agent |
| chlorambucil | anticancer agent |
| CI-994 | anticancer agent |
| Cisplatin | anticancer agent |
| Cladribine | anticancer agent |
| CS-682 | anticancer agent |
| cyclophosphamide | anticancer agent |
| cytarabine | anticancer agent |
| Cytarabine HCl | anticancer agent |
| cytoskeletal disruptor | anticancer agent |
| D2163 | anticancer agent |
| dacarbazine | anticancer agent |
| Dactinomycin | anticancer agent |
| daunorubicin | anticancer agent |
| Daunorubicin HCl | anticancer agent |
| DepoCyt | anticancer agent |
| Dexifosamide | anticancer agent |
| Docetaxel | anticancer agent |
| Dolastain | anticancer agent |
| Doxifluridine | anticancer agent |
| Doxorubicin | anticancer agent |
| DX8951f | anticancer agent |
| E 7070 | anticancer agent |
| EGFR | anticancer agent |
| Epirubicin | anticancer agent |
| epothilone | anticancer agent |
| erlotinib | anticancer agent |
| Estramustine phosphate sodium | anticancer agent |
| Etoposide (VP16-213) | anticancer agent |
| Farnesyl Transferase Inhibitor | anticancer agent |
| FK 317 | anticancer agent |
| Flavopiridol | anticancer agent |
| Floxuridine | anticancer agent |
| Fludarabine | anticancer agent |
| Fluorouracil (5-FU) | anticancer agent |
| Flutamide | anticancer agent |
| Fragyline | anticancer agent |
| gefitinib | anticancer agent |
| Gemcitabine | anticancer agent |
| Hexamethylmelamine (HMM) | anticancer agent |
| histone deacetylase inhibitor | anticancer agent |
| hydroxyurea | anticancer agent |
| Hydroxyurea (hydroxycarbamide) | anticancer agent |
| idarubicin | anticancer agent |
| Ifosfamide | anticancer agent |
| imatinib | anticancer agent |
| Interferon Alfa-2a | anticancer agent |
| Interferon Alfa-2b | anticancer agent |
| Irinotecan | anticancer agent |
| ISI 641 | anticancer agent |
| kinase inhibitor | anticancer agent |
| Krestin | anticancer agent |
| Lemonal DP 2202 | anticancer agent |
| Leuprolide acetate (LHRH-releasing factor analogue) | anticancer agent |
| Levamisole | anticancer agent |
| LiGLA (lithium-gamma linolenate) | anticancer agent |
| Lodine Seed | anticancer agent |
| Lometexol | anticancer agent |
| Lomustine (CCNU) | anticancer agent |
| Marimistat | anticancer agent |
| mechlorethamine | anticancer agent |
| Mechlorethamine HCl (nitrogen mustard) | anticancer agent |
| Megestrol acetate | anticancer agent |
| Meglamine GLA | anticancer agent |
| melphalan | anticancer agent |
| Mercaptopurine | anticancer agent |
| Mesna | anticancer agent |
| methotrexate | anticancer agent |
| methyl glyoxal bis-guanylhydrazone (MGBG) | anticancer agent |
| Mitoguazone (methyl-GAG | anticancer agent |
| Mitotane (o.p′-DDD) | anticancer agent |
| Mitoxantrone HCl | anticancer agent |
| mitozantrone | anticancer agent |
| MMI 270 | anticancer agent |
| MMP | anticancer agent |
| MTA/LY 231514 | anticancer agent |
| MTX | anticancer agent |
| nitrosourea | anticancer agent |
| nucleotide analogue | anticancer agent |
| nucleotide precursor analogue | anticancer agent |
| ODN 698 | anticancer agent |
| OK-432 | anticancer agent |
| Oral Platinum | anticancer agent |
| Oral Taxoid | anticancer agent |
| oxaliplatin | anticancer agent |
| paclitaxel | anticancer agent |
| PARP Inhibitor | anticancer agent |
| PD 183805 | anticancer agent |
| Pentostatin (2′ deoxycoformycin) | anticancer agent |
| PKC 412 | anticancer agent |
| platinum based chemotherapeutic | anticancer agent |
| Plicamycin | anticancer agent |
| Procarbazine HCl | anticancer agent |
| PSC 833 | anticancer agent |
| Ralitrexed | anticancer agent |
| RAS Farnesyl Transferase Inhibitor | anticancer agent |
| RAS Oncogene Inhibitor | anticancer agent |
| retinoids | anticancer agent |
| romidepsin | anticancer agent |
| Semustine (methyl-CCNU) | anticancer agent |
| Streptozocin | anticancer agent |
| Suramin | anticancer agent |
| tafluposide | anticancer agent |
| Tamoxifen citrate | anticancer agent |
| taxane | anticancer agent |
| Taxane Analog | anticancer agent |
| taxotere | anticancer agent |
| Temozolomide | anticancer agent |
| Teniposide (VM-26) | anticancer agent |
| Thioguanine | anticancer agent |
| Thiotepa | anticancer agent |
| tioguanine | anticancer agent |
| topoisomerase I inhibitor | anticancer agent |
| topoisomerase II inhibitor | anticancer agent |
| Topotecan | anticancer agent |
| tretinoin | anticancer agent |
| Tyrosine Kinase | anticancer agent |
| UFT (Tegafur/Uracil) | anticancer agent |
| Valrubicin | anticancer agent |
| vemurafenib | anticancer agent |
| vinblastine | anticancer agent |
| Vinblastine sulfate | anticancer agent |
| vinca alkaloid | anticancer agent |
| vinca alkaloid derivative | anticancer agent |
| vincristine | anticancer agent |
| vindesine | anticancer agent |
| Vindesine sulfate | anticancer agent |
| vinorelbine | anticancer agent |
| vismodegib | anticancer agent |
| vorinostat | anticancer agent |
| VX-710 | anticancer agent |
| VX-853 | anticancer agent |
| YM 116 | anticancer agent |
| ZD 0101 | anticancer agent |
| ZD 0473/Anormed | anticancer agent |
| ZD 1839 | anticancer agent |
| ZD 9331 | anticancer agent |
| 2-dimensional tissue | biological |
| 3-dimensional tissue | biological |
| adenovirus | biological |
| adipose tissue-derived mesenchymal stem cell | biological |
| bacteria | biological |
| bone mesenchymal stem cell | biological |
| cardiac mesenchymal stem cell | biological |
| cells | biological |
| chicken dorsal root ganglion | biological |
| complex carbohydrate | biological |
| deoxyribonucleic acid | biological |
| embryonic stem cell | biological |
| fibroblast | biological |
| fungi | biological |
| gene | biological |
| hematopoietic stem cell | biological |
| human corneal epithelial cell | biological |
| human corneal stromal stem cell | biological |
| human small intestinal enteroids | biological |
| lentivirus | biological |
| limbal epithelial stem cell | biological |
| lipids | biological |
| macromolecule | biological |
| mesenchymal stem cell | biological |
| microbe | biological |
| microbiome | biological |
| microorganism | biological |
| neural stem cells | biological |
| oligonucleotide | biological |
| oral keratinocyte | biological |
| organ | biological |
| organism | biological |
| periodontal ligament stem cells | biological |
| polymer | biological |
| probiotic | biological |
| proteins | biological |
| ribonucleic acid | biological |
| spore | biological |
| stem cell | biological |
| symbiote | biological |
| T cell | biological |
| tissue | biological |
| transfected fibrobast | biological |
| vesicle | biological |
| viral particle | biological |
| virus | biological |
| abequose | carbohydrate |
| arabinose | carbohydrate |
| cellobiose | carbohydrate |
| derivative of a monosaccaride | carbohydrate |
| disaccharide | carbohydrate |
| fructose | carbohydrate |
| fucose | carbohydrate |
| galactosamine | carbohydrate |
| galactose | carbohydrate |
| glucosamine | carbohydrate |
| glucose | carbohydrate |
| glucuronic acid | carbohydrate |
| iduronic acid | carbohydrate |
| lactose | carbohydrate |
| maltose | carbohydrate |
| mannose | carbohydrate |
| monosaccharide | carbohydrate |
| muramic acid | carbohydrate |
| N-acetylgalactosamine | carbohydrate |
| N-acetylglucosamine | carbohydrate |
| N-acetylmuramic acid | carbohydrate |
| N-acetylneuraminic acid | carbohydrate |
| oligosaccharide | carbohydrate |
| rhamnose | carbohydrate |
| ribose | carbohydrate |
| sialic acid | carbohydrate |
| sucrose | carbohydrate |
| treahalose | carbohydrate |
| xylose | carbohydrate |
| adipose tissue-derived mesenchymal stem cell | cell |
| periodontal ligament stem cell | cell |
| human small intestinal enteroid | cell |
| oral keratinocyte | cell |
| fibroblast | cell |
| transfected fibrobast | cell |
| 2-dimensional tissue | cell |
| 3-dimensional tissue | cell |
| T cell | cell |
| embryonic stem cell | cell |
| neural stem cell | cell |
| mesenchymal stem cell | ceil |
| hematopoietic stem cell | cell |
| osteoblast | cell |
| osteoclast | cell |
| osteocyte | cell |
| neuron | cell |
| glial cell | cell |
| chondrocyte | cell |
| photoreceptor cell | cell |
| cone cell | cell |
| rod cell | cell |
| corneal cell | cell |
| keratocyte | cell |
| corneal endothelial cell | cell |
| brain-derived neutrophic factor (BDNF) | cytokine |
| cardiotrophin 1 (CTF1) | cytokine |
| cardiotrophin-like cytokine factor 1 (CLCF1) | cytokine |
| cell signal molecule | cytokine |
| chemokine | cytokine |
| ciliary neutrophic factor (CNTF) | cytokine |
| erythropoietin (EPO) | cytokine |
| fibroblast growth factor acidic (FGFa) | cytokine |
| fibroblast growth factor basic (FGFb) | cytokine |
| IL- 18 | cytokine |
| IL-10 | cytokine |
| IL-11 | cytokine |
| IL-12 | cytokine |
| IL-13 | cytokine |
| IL-14 | cytokine |
| IL-15 | cytokine |
| IL-16 | cytokine |
| IL-17 | cytokine |
| IL-19 | cytokine |
| IL-1α | cytokine |
| IL-1β | cytokine |
| IL-2 | cytokine |
| IL-20 | cytokine |
| IL-21 | cytokine |
| IL-22 | cytokine |
| IL-23 | cytokine |
| IL-27 | cytokine |
| 1L-3 | cytokine |
| IL-4 | cytokine |
| IL-5 | cytokine |
| IL-6 | cytokine |
| IL-7 | cytokine |
| IL-8 | cytokine |
| IL-9 | cytokine |
| interferon | cytokine |
| interferon-α1 | cytokine |
| interleukin | cytokine |
| interleukin-1 receptor antagonist (IL-1RA) | cytokine |
| keratinocyte growth factor 1 | cytokine |
| keratinocyte growth factor 2 (KGF) | cytokine |
| kit ligand/stem cell factor (KITLG) | cytokine |
| leptin (LEP) | cytokine |
| leukemia inhibitory factor (LIF) | cytokine |
| lymphokine | cytokine |
| matrix metalloproteinase (MMP) | cytokine |
| monokine | cytokine |
| nerve growth factor (NGF) | cytokine |
| oncostatin M (OSM) | cytokine |
| prolactin (PRL) | cytokine |
| TGFβ | cytokine |
| tissue inhibitor of metalloproteinase (TIMP) | cytokine |
| transforming growth factor (TGF) α (TGFα) | cytokine |
| tumor necrosis factor α (TNFα) | cytokine |
| diacylglycerol | fat |
| diglycerides | fat |
| ergosterol | fat |
| fat-soluble vitamin | fat |
| glycerol monostearate | fat |
| glycerophospholipid | fat |
| glyceryl hydroxystearate | fat |
| hopanoid | fat |
| hydroxy steroid | fat |
| monoglyceride | fat |
| monolaurin | fat |
| oil | fat |
| palmitin | fat |
| phosphatidic acid | fat |
| phosphatidylcholine | fat |
| phosphatidylethanolamine | fat |
| phosphatidylserine | fat |
| phosphoinositides | fat |
| phospholipids | fat |
| phosphosphingolipids | fat |
| phytosterol | fat |
| sphingolipid | fat |
| sphingomyelin | fat |
| stearin | fat |
| sterol | fat |
| triglyceride | fat |
| triolein | fat |
| wax | fat |
| fatty acid | fatty acid |
| essential fatty acid | fatty acid |
| omega-3 fatty acid | fatty acid |
| lineoleic acid | fatty acid |
| omega-6 fatty acid | fatty acid |
| docosahezaenoic acid | fatty acid |
| arachidonic acid | fatty acid |
| omega-9 fatty acid | fatty acid |
| Hexadecatrienoic acid (HTA) | fatty acid |
| α-Linolenic acid (ALA) | fatty acid |
| Stearidonic acid (SDA) | fatty acid |
| Eicosatrienoic acid (ETE) | fatty acid |
| Eicosatetraenoic acid (ETA) | fatty acid |
| Eicosapentaenoic acid (EPA) | fatty acid |
| Heneicosapentaenoic acid (HPA) | fatty acid |
| Docosapentaenoic acid (DPA), | fatty acid |
| Clupanodonic acid | fatty acid |
| Docosahexaenoic acid (DHA) | fatty acid |
| Tetracosapentaenoic acid | fatty acid |
| Tetracosahexaenoic acid (Nisinic acid) | fatty acid |
| 5-Dodecenoic acid | fatty acid |
| 7-Tetradecenoic acid | fatty acid |
| 9-Hexadecenoic acid | fatty acid |
| 11-Octadecenoic acid | fatty acid |
| 13-Eicosenoic acid | fatty acid |
| 15-Docosenoic acid | fatty acid |
| 17-Tetracosenoic acid | fatty acid |
| Linoleic acid (LA) | fatty acid |
| Gamma-linolenic acid (GLA) | fatty acid |
| Calendic acid | fatty acid |
| Eicosadienoic acid | fatty acid |
| Dihomo-gamma-linolenic acid (DGLA) | fatty acid |
| Arachidonic acid (AA, ARA) | fatty acid |
| Docosadienoic acid | fatty acid |
| Adrenic acid | fatty acid |
| Osbond acid | fatty acid |
| Tetracosatetraenoic acid | fatty acid |
| Tetracosapentaenoic acid | fatty acid |
| oleic acid | fatty acid |
| elaidic acid | fatty acid |
| gondoic acid | fatty acid |
| mead acid | fatty acid |
| erucic acid | fatty acid |
| nervonic acid | fatty acid |
| ximenic acid | fatty acid |
| bone morphogenic protein | protein |
| bone morphogenic-like protein | protein |
| epidermal growth factor | protein |
| fibroblast growth factor | protein |
| insulin like growth factor I | protein |
| insulin like growth factor II | protein |
| transforming growth factor | protein |
| biotin (vitamin B7) | health supplement |
| iodine | health supplement |
| niacin (vitamin B3) | health supplement |
| pantothenic acid (vitamin B5) | health supplement |
| phosphorus | health supplement |
| riboflavin (vitamin B2) | health supplement |
| selenium | health supplement |
| thiamine (vitamin B1) | health supplement |
| vanadium | health supplement |
| vitamin A | health supplement |
| vitamin B | health supplement |
| vitamin B12 | health supplement |
| vitamin B6 | health supplement |
| vitamin B9 | health supplement |
| vitamin C | health supplement |
| vitamin D | health supplement |
| vitamin E | health supplement |
| vitamin K | health supplement |
| allspice berry essential oil | herbal preparation |
| angelica seed essential oil | herbal preparation |
| anise seed essential oil | herbal preparation |
| basil | herbal preparation |
| basil essential oil | herbal preparation |
| bay essential oil | herbal preparation |
| bay laurel | herbal preparation |
| bay laurel essential oil | herbal preparation |
| bergamot essential oil | herbal preparation |
| blood orange essential oil | herbal preparation |
| borage | herbal preparation |
| camphor essential oil | herbal preparation |
| caraway | herbal preparation |
| caraway seed essential oil | herbal preparation |
| cardamom seed essential oil | herbal preparation |
| carrot seed essential oil | herbal preparation |
| cassia essential oil | herbal preparation |
| catnip | herbal preparation |
| catnip essential oil | herbal preparation |
| cedarwood essential oil | herbal preparation |
| celery seed essential oil | herbal preparation |
| chamomile german essential oil | herbal preparation |
| chamomile roman essential oil | herbal preparation |
| chervil | herbal preparation |
| chives | herbal preparation |
| cilantro | herbal preparation |
| cinnamon bark essential oil | herbal preparation |
| cinnamon leaf essential oil | herbal preparation |
| citronella essential oil | herbal preparation |
| clary sage essential oil | herbal preparation |
| clove bud essential oil | herbal preparation |
| cold infusion | herbal preparation |
| compresses | herbal preparation |
| cordial | herbal preparation |
| coriander seed essential oil | herbal preparation |
| cumin | herbal preparation |
| cypress essential oil | herbal preparation |
| decoctions | herbal preparation |
| dill | herbal preparation |
| elemi essential oil | herbal preparation |
| epazote | herbal preparation |
| essential oils | herbal preparation |
| eucalyptus essential oil | herbal preparation |
| fennel | herbal preparation |
| fennel essential oil | herbal preparation |
| fir needle essential oil | herbal preparation |
| flower essence | herbal preparation |
| frankincense essential oil | herbal preparation |
| garlic | herbal preparation |
| geranium essential oil | herbal preparation |
| ginger essential oil | herbal preparation |
| granule | herbal preparation |
| grapefruit pink essential oil | herbal preparation |
| helichrysum essential oil | herbal preparation |
| herbal wine | herbal preparation |
| hop essential oil | herbal preparation |
| hyssop essential oil | herbal preparation |
| jasmine absolute oil | herbal preparation |
| juniper berry essential oil | herbal preparation |
| labdanum essential oil | herbal preparation |
| lavender | herbal preparation |
| lavender absolute oil | herbal preparation |
| lemon balm | herbal preparation |
| lemon essential oil | herbal preparation |
| lemon verbena | herbal preparation |
| lemongrass | herbal preparation |
| lemongrass essential oil | herbal preparation |
| lime essential oil | herbal preparation |
| lovage | herbal preparation |
| magnolia essential oil | herbal preparation |
| mandarin essential oil | herbal preparation |
| margoram essential oil | herbal preparation |
| marjoram | herbal preparation |
| Melissa essential oil | herbal preparation |
| mints | herbal preparation |
| mugward essential oil | herbal preparation |
| myrrh essential oil | herbal preparation |
| myrtle essential oil | herbal preparation |
| nasturtium | herbal preparation |
| neroli essential oil | herbal preparation |
| niaouli essential oil | herbal preparation |
| nutmeg essential oil | herbal preparation |
| ointment | herbal preparation |
| orange sweet essential oil | herbal preparation |
| oregano | herbal preparation |
| oregano essential oil | herbal preparation |
| palmarosa essential oil | herbal preparation |
| parsley | herbal preparation |
| patchouli essential oil | herbal preparation |
| pennyroyal essential oil | herbal preparation |
| pepper black essential oil | herbal preparation |
| peppermint essential oil | herbal preparation |
| petitgram essential oil | herbal preparation |
| pine needle essential oil | herbal preparation |
| pink lotus absolute oil | herbal preparation |
| poultice | herbal preparation |
| radiata essential oil | herbal preparation |
| ravensara essential oil | herbal preparation |
| rose absolute oil | herbal preparation |
| rose essential oil | herbal preparation |
| rosemary | herbal preparation |
| rosemary essential oil | herbal preparation |
| rosewood essential oil | herbal preparation |
| sage | herbal preparation |
| sage essential oil | herbal preparation |
| salad burnet | herbal preparation |
| salve | herbal preparation |
| sambac absolute oil | herbal preparation |
| sandalwood essential oil | herbal preparation |
| savory | herbal preparation |
| scented geranium | herbal preparation |
| sitz bath | herbal preparation |
| soak | herbal preparation |
| sorrel | herbal preparation |
| spearmint essential oil | herbal preparation |
| spikenard essential oil | herbal preparation |
| spruce essential oil | herbal preparation |
| star anise essential oil | herbal preparation |
| suppository | herbal preparation |
| sweet annie essential oil | herbal preparation |
| syrup | herbal preparation |
| tangerine essential oil | herbal preparation |
| tarragon | herbal preparation |
| tea | herbal preparation |
| tea tree essential oil | herbal preparation |
| thyme | herbal preparation |
| thyme red essential oil | herbal preparation |
| tincture | herbal preparation |
| verbena essential oil | herbal preparation |
| vetiver essential oil | herbal preparation |
| white lotus absolute oil | herbal preparation |
| wintergreen essential oil | herbal preparation |
| wormwood essential oil | herbal preparation |
| yarrow essential oil | herbal preparation |
| ylang essential oil | herbal preparation |
| 3-ketodesogestrel | hormone |
| allopregnanolone | hormone |
| androgen | hormone |
| androstenediol | hormone |
| androstenedione | hormone |
| chlormadinone acetate | hormone |
| cholesterol | hormone |
| conjugated estrogen | hormone |
| dehydroepiandrosterone | hormone |
| dexamethasone | hormone |
| dihydrotestosterone | hormone |
| drospirorenone | hormone |
| estradiol ester | hormone |
| estradiols | hormone |
| estriol | hormone |
| estriol succinate | hormone |
| estrogen | hormone |
| estrone | hormone |
| estrone sulfate | hormone |
| ethinyl estradiol | hormone |
| gestodene | hormone |
| glucocoriticoid | hormone |
| levonorgestrel | hormone |
| mestranol | hormone |
| mineralocorticoid | hormone |
| norethisterone acetate | hormone |
| norgestrel | hormone |
| polyestriol phosphate | hormone |
| progesterone | hormone |
| progestogen | hormone |
| testosterone | hormone |
| calcium oxide | ion, metal, or mineral |
| iron oxide | ion, metal, or mineral |
| phosphorus oxide | ion, metal, or mineral |
| iodine oxide | ion, metal, or mineral |
| magnesium oxide | ion, metal, or mineral |
| zinc oxide | ion, metal, or mineral |
| selenium oxide | ion, metal, or mineral |
| copper oxide | ion, metal, or mineral |
| manganese oxide | ion, metal, or mineral |
| chromium oxide | ion, metal, or mineral |
| molybdenum oxide | ion. metal, or mineral |
| gold oxide | ion, metal, or mineral |
| potassium oxide | ion, metal, or mineral |
| Advexin (Introgen) | nucleic acid |
| nucleic acid vaccines | nucleic acid |
| fomivirsen sodium (Isis Pharmaceuticals) | nucleic acid |
| MG98 | nucleic acid |
| ISIS 5132 | nucleic acid |
| DNAzyme | nucleic acid |
| 2,5-diketopiperazine | oxidant or antioxidant |
| antioxidant | oxidant or antioxidant |
| melanin | oxidant or antioxidant |
| oxidants | oxidant or antioxidant |
| quarternary ammonium chitosan | oxidant or antioxidant |
| ion | oxidant or antioxidant |
| mineral | oxidant or antioxidant |
| vitamin | oxidant or antioxidant |
| protein | oxidant or antioxidant |
| hydrogen peroxide | oxidant or antioxidant |
| ozone | oxidant or antioxidant |
| nitric acid | oxidant or antioxidant |
| sulfuric acid | oxidant or antioxidant |
| oxygen | oxidant or antioxidant |
| sodium perborate | oxidant or antioxidant |
| nitrous oxide | oxidant or antioxidant |
| potassium nitrate | oxidant or antioxidant |
| sodium bismuthate | oxidant or antioxidant |
| hypochlorite | oxidant or antioxidant |
| bleach | oxidant or antioxidant |
| halogen | oxidant or antioxidant |
| Cl2 | oxidant or antioxidant |
| F2 | oxidant or antioxidant |
| endogenous oxidant | oxidant or antioxidant |
| exogenous oxidant | oxidant or antioxidant |
| hydroxide | oxidant or antioxidant |
| singlet oxygen | oxidant or antioxidant |
| superoxide anion | oxidant or antioxidant |
| hydroxy one radical | oxidant or antioxidant |
| reactive oxygen species | oxidant or antioxidant |
| vitamin A | oxidant or antioxidant |
| beta-carotene | oxidant or antioxidant |
| carotenoid | oxidant or antioxidant |
| vitamin C | oxidant or antioxidant |
| ascorbic acid | oxidant or antioxidant |
| vitamin E | oxidant or antioxidant |
| tocopherol | oxidant or antioxidant |
| tocotrienol | oxidant or antioxidant |
| selenium | oxidant or antioxidant |
| glutatione peroxidase | oxidant or antioxidant |
| zinc | oxidant or antioxidant |
| catalase | oxidant or antioxidant |
| superoxide dismutase | oxidant or antioxidant |
| copper | oxidant or antioxidant |
| manganese | oxidant or antioxidant |
| glutathione | oxidant or antioxidant |
| polyphenol | oxidant or antioxidant |
| tirilazad | oxidant or antioxidant |
| allupurinol | oxidant or antioxidant |
| acetylcysteine | oxidant or antioxidant |
| lipoic acid | oxidant or antioxidant |
| carotene | oxidant or antioxidant |
| ubiquinol | oxidant or antioxidant |
| BHA | oxidant or antioxidant |
| BHT | oxidant or antioxidant |
| Anidulafungin | peptide |
| Atosiban acetate | peptide |
| Bacitracin | peptide |
| Bivalirudin | peptide |
| Bortezomib | peptide |
| Buserelin | peptide |
| Calcitonin | peptide |
| Captopril | peptide |
| Carbetocin acetate | peptide |
| Caspofungin | peptide |
| Cetrorelix acetate | peptide |
| Colistin | peptide |
| cyclic dipeptide | peptide |
| cyclic peptide | peptide |
| Cyclosporine | peptide |
| Daptomycin | peptide |
| Degarelix acetate | peptide |
| Enalapril maleate | peptide |
| Enfuvirtide | peptide |
| Eptifibatide | peptide |
| Exenatide | peptide |
| Glutathione | peptide |
| Goserelin | peptide |
| Human calcitonin | peptide |
| Ianreotide acetate | peptide |
| Icatibant acetate | peptide |
| Lepirudin | peptide |
| Liraglutide | peptide |
| Lisinopril | peptide |
| Lypressin | peptide |
| Nafarelin acetate | peptide |
| Nesiritide | peptide |
| Oxytocin | peptide |
| RGD peptide | peptide |
| r-hirudin | peptide |
| Salmon calcitonin | peptide |
| Saralasin acetate | peptide |
| Somatostatin acetate | peptide |
| Spaglumat magnesium | peptide |
| Thymalfasin | peptide |
| Tirofiban | peptide |
| Vapreotide acetate | peptide |
| Ziconotide | peptide |
| adrenocorticotropic hormone (ACTH) | protein |
| Alpha interferon | protein |
| antibody | protein |
| antigen | protein |
| Beta interferon | protein |
| Bone morphogenetic protein (BMP) | protein |
| bone-morphogenic protein 2 | protein |
| chimeric protein | protein |
| Coagulation Factor IX | protein |
| Colony stimulating growth factor (CSF) | protein |
| Desmopressin | protein |
| Etanercept | protein |
| Factor IX | protein |
| Factor VII | protein |
| Factor VIII | protein |
| Follicle stimulating hormone (FSH) | protein |
| Gamma interferon | protein |
| gastrin prolactin | protein |
| Granulocyte colony-stimulating factor (G-CSF) | protein |
| Granulocyte macropitage colony stimulating | protein |
| factor (GM-CSF) | |
| growth hormone (GH) | protein |
| Human chorionic gonadotropin (HCG) | protein |
| infliximab | protein |
| insulin | protein |
| Insulin glargine | protein |
| Insulin-like growth factor (IGF) | protein |
| kallikrein | protein |
| kerantinocyte growth factor (KGF) | protein |
| Luteinizing hormone (LH) | protein |
| Macrophage colony stimulating factor (M-CSF) | protein |
| Neurotrophic growth factor (NGF) | protein |
| obesity protein (leptin) | protein |
| Octreotide | protein |
| Osteoprotegerin (OPG) | protein |
| pancreatic RNAase | protein |
| Pegfilgrastim | protein |
| peptides | protein |
| platelet activating factor acetyl hydrolase | protein |
| Platelet-derived growth factor (PDGF) | protein |
| processed silk | protein |
| sericin | protein |
| silk | protein |
| silk fibroin | protein |
| Stem cell factor (SCF) | protein |
| streptokinase | protein |
| Superoxide dismutase (SOD) | protein |
| synthetic protein | protein |
| thrombopoietin | protein |
| Thyroid stimulating hormone (TSH) | protein |
| tissue plasminogen activator (TPA) | protein |
| Tumor necrosis factor (TNF) | protein |
| tumor necrosis factor binding protein (TNFbp) | protein |
| urokinase | protein |
| Vascular endothelial growth factor (VEGF) | protein |
| antibacterial agent | small molecule |
| antifungal agent | small molecule |
| antimalarial agent | small molecule |
| antiseptic | small molecule |
| nonsteroidal anti-inflammatory drugs (NSAIDs) | small molecule |
| stimulant | small molecule |
| tranquilizers | small molecule |
| acetaminophen | small molecule - analgesic |
| | agent |
| alcohols | small molecule - analgesic |
| | agent |
| alcuronium | small molecule - analgesic |
| | agent |
| alfentanyl | small molecule - analgesic |
| | agent |
| amethocaine | small molecule - analgesic |
| | agent |
| amobarbital | small molecule - analgesic |
| | agent |
| anileridine | small molecule - analgesic |
| | agent |
| atracurium | small molecule - analgesic |
| | agent |
| bupivacaine | small molecule - analgesic |
| | agent |
| buprenorphine | small molecule - analgesic |
| | agent |
| butorphanol | small molecule - analgesic |
| | agent |
| cannabinoid | small molecule - analgesic |
| | agent |
| cannabis | small molecule - analgesic |
| | agent |
| cisatracurium | small molecule - analgesic |
| | agent |
| cocaine | small molecule - analgesic |
| | agent |
| codiene | small molecule - analgesic |
| | agent |
| COX-2 inhibitor | small molecule - analgesic |
| | agent |
| decamethonium | small molecule - analgesic |
| | agent |
| diacetyl morphine | small molecule - analgesic |
| | agent |
| diamorphine | small molecule - analgesic |
| | agent |
| diazepam | small molecule - analgesic |
| | agent |
| dibucaine | small molecule - analgesic |
| | agent |
| doxacurium | small molecule - analgesic |
| | agent |
| etomidate | small molecule - analgesic |
| | agent |
| fentanyl | small molecule - analgesic |
| | agent |
| gallamine | small molecule - analgesic |
| | agent |
| hydrocodone | small molecule - analgesic |
| | agent |
| hydromorphone | small molecule - analgesic |
| | agent |
| ketamine | small molecule - analgesic |
| | agent |
| levobupivacaine | small molecule - analgesic |
| | agent |
| levorphanol | small molecule - analgesic |
| | agent |
| lidocaine | small molecule - analgesic |
| | agent |
| lorazepam | small molecule - analgesic |
| | agent |
| meperidine (pethidine) | small molecule - analgesic |
| | agent |
| mepivacaine | small molecule - analgesic |
| | agent |
| methadone | small molecule - analgesic |
| | agent |
| methohexital | small molecule - analgesic |
| | agent |
| metocurine | small molecule - analgesic |
| | agent |
| midazolam | small molecule - analgesic |
| | agent |
| morphine | small molecule - analgesic |
| | agent |
| morphine glucuronide | small molecule - analgesic |
| | agent |
| morphine sulfate | small molecule - analgesic |
| | agent |
| nalbuphine | small molecule - analgesic |
| | agent |
| NSAID | small molecule - analgesic |
| | agent |
| opioid agonist | small molecule - analgesic |
| | agent |
| opioid antagonist | small molecule - analgesic |
| | agent |
| opioids | small molecule - analgesic |
| | agent |
| oxycodone | small molecule - analgesic |
| | agent |
| oxymorphone | small molecule - analgesic |
| | agent |
| pancuronium | small molecule - analgesic |
| | agent |
| pentazocine | small molecule - analgesic |
| | agent |
| pipecuronium | small molecule - analgesic |
| | agent |
| prilocaine | small molecule - analgesic |
| | agent |
| procaine | small molecule - analgesic |
| | agent |
| propoxyphene | small molecule - analgesic |
| | agent |
| rapacuronium | small molecule - analgesic |
| | agent |
| remifentanil | small molecule - analgesic |
| | agent |
| rocuronium | small molecule - analgesic |
| | agent |
| ropivacaine | small molecule - analgesic |
| | agent |
| succinylcholine | small molecule - analgesic |
| | agent |
| sufentanil | small molecule - analgesic |
| | agent |
| thiamylal | small molecule - analgesic |
| | agent |
| thiopental | small molecule - analgesic |
| | agent |
| tubocurarine | small molecule - analgesic |
| | agent |
| vecuronium | small molecule - analgesic |
| | agent |
| amikacin | small molecule - antibacterial |
| | agent |
| amoxicillin | small molecule - antibacterial |
| | agent |
| ampicillin | small molecule - antibacterial |
| | agent |
| azithromycin | small molecule - antibacterial |
| | agent |
| azlocillin | small molecule - antibacterial |
| | agent |
| aztreonam | small molecule - antibacterial |
| | agent |
| capreomycin | small molecule - antibacterial |
| | agent |
| carbenicillin | small molecule - antibacterial |
| | agent |
| cefaclor | small molecule - antibacterial |
| | agent |
| cefadroxil | small molecule - antibacterial |
| | agent |
| cefalexin | small molecule - antibacterial |
| | agent |
| cefalotin | small molecule - antibacterial |
| | agent |
| cefamandole | small molecule - antibacterial |
| | agent |
| cefazolin | small molecule - antibacterial |
| | agent |
| cefdinir | small molecule - antibacterial |
| | agent |
| cefditoren | small molecule - antibacterial |
| | agent |
| cefepime | small molecule - antibacterial |
| | agent |
| cefixime | small molecule - antibacterial |
| | agent |
| cefoperazone | small molecule - antibacterial |
| | agent |
| cefotaxime | small molecule - antibacterial |
| | agent |
| cefoxitin | small molecule - antibacterial |
| | agent |
| cefpodoxime | small molecule - antibacterial |
| | agent |
| cefprozil | small molecule - antibacterial |
| | agent |
| ceftaroline fosamil | small molecule - antibacterial |
| | agent |
| ceftazidime | small molecule - antibacterial |
| | agent |
| ceftibuten | small molecule - antibacterial |
| | agent |
| ceftizoxime | small molecule - antibacterial |
| | agent |
| ceftobiprole | small molecule - antibacterial |
| | agent |
| ceftriaxone | small molecule - antibacterial |
| | agent |
| cefuroxime | small molecule - antibacterial |
| | agent |
| cilastatin | small molecule - antibacterial |
| | agent |
| ciprofolaxin | small molecule - antibacterial |
| | agent |
| clarithromycin | small molecule - antibacterial |
| | agent |
| clindamycin | small molecule - antibacterial |
| | agent |
| clofazimine | small molecule - antibacterial |
| | agent |
| cloxacillin | small molecule - antibacterial |
| | agent |
| cycloserine | small molecule - antibacterial |
| | agent |
| dalbavancin | small molecule - antibacterial |
| | agent |
| dapsone | small molecule - antibacterial |
| | agent |
| demeclocycline | small molecule - antibacterial |
| | agent |
| dicloxacillin | small molecule - antibacterial |
| | agent |
| dirithromycin | small molecule - antibacterial |
| | agent |
| doripenem | small molecule - antibacterial |
| | agent |
| doxycycline | small molecule - antibacterial |
| | agent |
| enoxacin | small molecule - antibacterial |
| | agent |
| ertapenem | small molecule - antibacterial |
| | agent |
| erythromycin | small molecule - antibacterial |
| | agent |
| ethambutol | small molecule - antibacterial |
| | agent |
| ethionamide | small molecule - antibacterial |
| | agent |
| flucloxacillin | small molecule - antibacterial |
| | agent |
| furazolidone | small molecule - antibacterial |
| | agent |
| gatifloxacin | small molecule - antibacterial |
| | agent |
| geldanamycin | small molecule - antibacterial |
| | agent |
| gemifloxacin | small molecule - antibacterial |
| | agent |
| gentamicin | small molecule - antibacterial |
| | agent |
| grepafloxacin | small molecule - antibacterial |
| | agent |
| herbimycin | small molecule - antibacterial |
| | agent |
| imipeneum | small molecule - antibacterial |
| | agent |
| isoniazid | small molecule - antibacterial |
| | agent |
| kanamycin | small molecule - antibacterial |
| | agent |
| levofloxacin | small molecule - antibacterial |
| | agent |
| linezolid | small molecule - antibacterial |
| | agent |
| linomycin | small molecule - antibacterial |
| | agent |
| lomefloxacin | small molecule - antibacterial |
| | agent |
| loracarbef | small molecule - antibacterial |
| | agent |
| mafenide | small molecule - antibacterial |
| | agent |
| meropenem | small molecule - antibacterial |
| | agent |
| methicillin | small molecule - antibacterial |
| | agent |
| mezlocillin | small molecule - antibacterial |
| | agent |
| minocycline | small molecule - antibacterial |
| | agent |
| moxifloxacin | small molecule - antibacterial |
| | agent |
| nafcillin | small molecule - antibacterial |
| | agent |
| nalidixic acid | small molecule - antibacterial |
| | agent |
| neomycin | small molecule - antibacterial |
| | agent |
| netilmicin | small molecule - antibacterial |
| | agent |
| nitrofurantoin | small molecule - antibacterial |
| | agent |
| norfloxacin | small molecule - antibacterial |
| | agent |
| ofloxacin | small molecule - antibacterial |
| | agent |
| oritavancin | small molecule - antibacterial |
| | agent |
| oxacillin | small molecule - antibacterial |
| | agent |
| oxytetracycline | small molecule - antibacterial |
| | agent |
| paromomycin | small molecule - antibacterial |
| | agent |
| penicillin | small molecule - antibacterial |
| | agent |
| penicillin G | small molecule - antibacterial |
| | agent |
| penicillin V | small molecule - antibacterial |
| | agent |
| piperacillin | small molecule - antibacterial |
| | agent |
| posizolid | small molecule - antibacterial |
| | agent |
| pyrazinamide | small molecule - antibacterial |
| | agent |
| radezolid | small molecule - antibacterial |
| | agent |
| rifampicin | small molecule - antibacterial |
| | agent |
| rifaximin | small molecule - antibacterial |
| | agent |
| roxithromycin | small molecule - antibacterial |
| | agent |
| sparfloxacin | small molecule - antibacterial |
| | agent |
| spectinomycin | small molecule - antibacterial |
| | agent |
| spiramycin | small molecule - antibacterial |
| | agent |
| streptomycin | small molecule - antibacterial |
| | agent |
| sulfacetamide | small molecule - antibacterial |
| | agent |
| sulfadiazine | small molecule - antibacterial |
| | agent |
| sulfadimethoxine | small molecule - antibacterial |
| | agent |
| sulfamethizole | small molecule - antibacterial |
| | agent |
| sulfamethoxazole | small molecule - antibacterial |
| | agent |
| sulfanilimide | small molecule - antibacterial |
| | agent |
| sulfasalazine | small molecule - antibacterial |
| | agent |
| sulfisoxazole | small molecule - antibacterial |
| | agent |
| teicoplanin | small molecule - antibacterial |
| | agent |
| telavancin | small molecule - antibacterial |
| | agent |
| telithromycin | small molecule - antibacterial |
| | agent |
| temafloxacin | small molecule - antibacterial |
| | agent |
| temocillin | small molecule - antibacterial |
| | agent |
| tetracycline | small molecule - antibacterial |
| | agent |
| ticarcillin | small molecule - antibacterial |
| | agent |
| tobramycin | small molecule - antibacterial |
| | agent |
| torezolid | small molecule - antibacterial |
| | agent |
| troleandomycin | small molecule - antibacterial |
| | agent |
| trovafloxacin | small molecule - antibacterial |
| | agent |
| vancomycin | small molecule - antibacterial |
| | agent |
| 5-fluorocytosine | small molecule - antifungal |
| abafungin | small molecule - antifungal |
| albaconazole | small molecule - antifungal |
| amorolfin | small molecule - antifungal |
| amphotericin B | small molecule - antifungal |
| benzoic acid | small molecule - antifungal |
| bifonazole | small molecule - antifungal |
| butenafine | small molecule - antifungal |
| butoconazole | small molecule - antifungal |
| candicidin | small molecule - antifungal |
| caspofungin | small molecule - antifungal |
| ciclopirox | small molecule - antifungal |
| clotrimazole | small molecule - antifungal |
| crystal violet | small molecule - antifungal |
| econazole | small molecule - antifungal |
| efinaconazole | small molecule - antifungal |
| epoxiconazole | small molecule - antifungal |
| fenticonazole | small molecule - antifungal |
| filipin | small molecule - antifungal |
| fluconazole | small molecule - antifungal |
| flucytosine | small molecule - antifungal |
| griseofulvin | small molecule - antifungal |
| haloprogin | small molecule - antifungal |
| hamycin | small molecule - antifungal |
| isavuconazole | small molecule - antifungal |
| isoconazole | small molecule - antifungal |
| itraconazole | small molecule - antifungal |
| ketoconazole | small molecule - antifungal |
| luliconazole | small molecule - antifungal |
| micafungin | small molecule - antifungal |
| miconazole | small molecule - antifungal |
| naftifine | small molecule - antifungal |
| natamycin | small molecule - antifungal |
| nystatin | small molecule - antifungal |
| omoconazole | small molecule - antifungal |
| oxiconazole | small molecule - antifungal |
| posaconazole | small molecule - antifungal |
| propiconazole | small molecule - antifungal |
| ravuconazole | small molecule - antifungal |
| rimocidin | small molecule - antifungal |
| sertaconazole | small molecule - antifungal |
| sulconazole | small molecule - antifungal |
| terbinafine | small molecule - antifungal |
| terconazole | small molecule - antifungal |
| tioconazole | small molecule - antifungal |
| tolnaftate | small molecule - antifungal |
| undecylenic acid | small molecule - antifungal |
| voriconazole | small molecule - antifungal |
| aminoquinoline | small molecule - antimalarial |
| amodiaquine | small molecule - antimalarial |
| antifolate | small molecule - antimalarial |
| artemether | small molecule - antimalarial |
| artemisinin derivative | small molecule - antimalarial |
| artemotil | small molecule - antimalarial |
| artesunate | small molecule - antimalarial |
| atovaquone | small molecule - antimalarial |
| biguanide | small molecule - antimalarial |
| bisphosphonate | small molecule - antimalarial |
| chloproguanil | small molecule - antimalarial |
| chloroquine | small molecule - antimalarial |
| cinchona alkaloid | small molecule - antimalarial |
| dermaseptin | small molecule - antimalarial |
| DHA-piperaquine | small molecule - antimalarial |
| diaminopyrimidine | small molecule - antimalarial |
| dihydroartemisinin | small molecule - antimalarial |
| doxycillin | small molecule - antimalarial |
| halofantrine | small molecule - antimalarial |
| lumefantrine | small molecule - antimalarial |
| melfoquine | small molecule - antimalarial |
| N-acetyl cysteine | small molecule - antimalarial |
| piperaquine | small molecule - antimalarial |
| primaquine | small molecule - antimalarial |
| proguanil | small molecule - antimalarial |
| pyremethamine | small molecule - antimalarial |
| pyronaridine | small molecule - antimalarial |
| quercitin | small molecule - antimalarial |
| quinidine | small molecule - antimalarial |
| quinine | small molecule - antimalarial |
| sulfadoxine-pyrimethamine | small molecule - antimalarial |
| sulfonamide | small molecule - antimalarial |
| tafenoquine | small molecule - antimalarial |
| trimethoprim | small molecule - antimalarial |
| choline salicylate | small molecule - antipyretic |
| magnesium salicylate | small molecule - antipyretic |
| metamizole | small molecule - antipyretic |
| nimesulide | small molecule - antipyretic |
| phenazone | small molecule - antipyretic |
| salicylate | small molecule - antipyretic |
| sodium salicylate | small molecule - antipyretic |
| aspirin | small molecule - NSAID |
| celecoxib | small molecule - NSAID |
| diclofenac | small molecule - NSAID |
| diflunisal | small molecule - NSAID |
| etodolac | small molecule - NSAID |
| fenoprofen | small molecule - NSAID |
| flurbiprofen | small molecule - NSAID |
| ibuprofen | small molecule - NSAID |
| indomethacin | small molecule - NSAID |
| ketoprofen | small molecule - NSAID |
| ketorolac | small molecule - NSAID |
| mechlofenamic acid | small molecule - NSAID |
| nabumetone | small molecule - NSAID |
| naproxen | small molecule - NSAID |
| oxaprozin | small molecule - NSAID |
| piroxicam | small molecule - NSAID |
| roficoxib | small molecule - NSAID |
| salsalate | small molecule - NSAID |
| sulindac | small molecule - NSAID |
| tolfenamic acid | small molecule - NSAID |
| tolmetin | small molecule - NSAID |
| sodium channel blocker | sodium channel blocker |
| alkaloid | sodium channel blocker |
| saxitoxin | sodium channel blocker |
| neosaxitoxin | sodium channel blocker |
| tetrodoxin | sodium channel blocker |
| class I antiarrhythmic agent | sodium channel blocker |
| quinidine | sodium channel blocker |
| procainamide | sodium channel blocker |
| disopryamide | sodium channel blocker |
| tocainide | sodium channel blocker |
| mexiletine | sodium channel blocker |
| proparacaine | sodium channel blocker |
| flecainide | sodium channel blocker |
| propafenone | sodium channel blocker |
| moricizine | sodium channel blocker |
| atorvastatin | statin |
| cerivastatin | statin |
| fluvastatin | statin |
| lovastatin | statin |
| mevastatin | statin |
| pitavastatin | statin |
| pravastatin | statin |
| rosuvastatin | statin |
| simvastatin | statin |
| statin | statin |
| steroid | steroid |
| corticosteroid | steroid |
| triamcinolone | steroid |
| cortisone | steroid |
| prednisone | steroid |
| methylprenisolone | steroid |
| prednisolone | steroid |
| betamethasone | steroid |
| dexamethasone | steroid |
| hydrocortisone | steroid |
| deflazacort | steroid |
| fludrocortisone | steroid |
| Anadrol | steroid |
| Anavar | steroid |
| Clenbuterol | steroid |
| Clomid | steroid |
| Cytomel | steroid |
| Deca Durabolin | steroid |
| Dianabol | steroid |
| Equipoise | steroid |
| Halotestin | steroid |
| Human Growth Hormone | steroid |
| Insulin | steroid |
| Lasix | steroid |
| Methyltestosterone | steroid |
| Nolvadex | steroid |
| Omnadren | steroid |
| Primobolan | steroid |
| Sustanon | steroid |
| Cypionate | steroid |
| Enanthate | steroid |
| Propionate | steroid |
| Testosterone | steroid |
| Trenbolone | steroid |
| Winstrol | steroid |
| Flunisolide | steroid |
| Budesonide | steroid |
| Mometasone | steroid |
| Ciclesonide | steroid |
| Fluticasone | steroid |
| Beclomethasone | steroid |
| glutocorticoid | steroid |
| minerolocorticoid | steroid |
| corticosterone | steroid |
| aldosterone | steroid |
| Hydrocortisone | steroid |
| methylprednisolone | steroid |
| prednisolone | steroid |
| prednisone | steroid |
| triamcinolone | steroid |
| Amcinonide | steroid |
| budesonide | steroid |
| desonide | steroid |
| fluocinolone acetonide | steroid |
| fluocinonide | steroid |
| halcinonide | steroid |
| triamcinolone acetonide | steroid |
| Beclometasone | steroid |
| betamethasone | steroid |
| dexamethasone | steroid |
| fluocortolone | steroid |
| halometasone | steroid |
| mometasone | steroid |
| Alclometasone dipropionate | steroid |
| betamethasone dipropionate | steroid |
| betamethasone valerate | steroid |
| clobetasol propionate | steroid |
| clobetasone butyrate | steroid |
| fluprednidene acetate | steroid |
| mometasone furoate | steroid |
| Ciclesonide | steroid |
| cortisone acetate | steroid |
| hydrocortisone aceponate | steroid |
| hydrocortisone acetate | steroid |
| hydrocortisone buteprate | steroid |
| hydrocortisone butyrate | steroid |
| hydrocortisone valerate | steroid |
| prednicarbate | steroid |
| tixocortol pivalate | steroid |
| 3,4-methylenedioxymethamphetamine | stimulant |
| amphetamines | stimulant |
| caffeine | stimulant |
| ephedrine | stimulant |
| mephedrone | stimulant |
| methamphetamine | stimulant |
| methylenedioxypyrovalerone | stimulant |
| methylphenidate | stimulant |
| nicotine | stimulant |
| phenylpropanolamine | stimulant |
| propylhexedrine | stimulant |
| pseudoephedrine | stimulant |
| imatinib (Gleevac) | therapeutic combination |
| all-trans-retinoic acid | therapeutic combination |
| monoclonal antibody treatment | therapeutic combination |
| gemtuzumab | therapeutic combination |
| ozogamicin | therapeutic combination |
| chemotherapy | therapeutic combination |
| chlorambucil | therapeutic combination |
| prednisone | therapeutic combination |
| prednisolone | therapeutic combination |
| vincristine | therapeutic combination |
| cytarabine | therapeutic combination |
| clofarabine | therapeutic combination |
| farnesyl transferase inhibitor | therapeutic combination |
| decitabine | therapeutic combination |
| inhibitor of MDR1 | therapeutic combination |
| rituximab | therapeutic combination |
| interferon-α | therapeutic combination |
| anthracycline drug | therapeutic combination |
| daunorubicin | therapeutic combination |
| idarubicin | therapeutic combination |
| L-asparaginase | therapeutic combination |
| doxorubicin | therapeutic combination |
| cyclophosphamide | therapeutic combination |
| bleomycin | therapeutic combination |
| fludarabine | therapeutic combination |
| etoposide | therapeutic combination |
| pentostatin | therapeutic combination |
| cladribine | therapeutic combination |
| bone marrow transplant | therapeutic combination |
| stem cell transplant | therapeutic combination |
| radiation therapy | therapeutic combination |
| anti-metabolite drug | therapeutic combination |
| methotrexate | therapeutic combination |
| 6-mercaptopurine | therapeutic combination |
| acepromazine | tranquilizer |
| alpha blockers | tranquilizer |
| alpha-adrenergic agonist | tranquilizer |
| antihistamine | tranquilizer |
| azapirone | tranquilizer |
| barbiturate | tranquilizer |
| benperidol | tranquilizer |
| benzamidine | tranquilizer |
| benzodiazepine | tranquilizer |
| beta-blocker | tranquilizer |
| bromantane | tranquilizer |
| bromperidol | tranquilizer |
| butyrophenone | tranquilizer |
| carbamates | tranquilizer |
| carpipramine | tranquilizer |
| chlorpromazine | tranquilizer |
| chlorprothixene | tranquilizer |
| clocapramine | tranquilizer |
| clopenthixol | tranquilizer |
| clorotepine | tranquilizer |
| cyamemazine | tranquilizer |
| diphenylbutylpiperidine | tranquilizer |
| dixyrazine | tranquilizer |
| droperidol | tranquilizer |
| emoxypine | tranquilizer |
| fabomotizole | tranquilizer |
| flupentixol | tranquilizer |
| fluphenazine | tranquilizer |
| fluspirilene | tranquilizer |
| gamma aminobutyric acid | tranquilizer |
| haloperidol | tranquilizer |
| inhalants | tranquilizer |
| levomepromazine | tranquilizer |
| loxapine | tranquilizer |
| mebicar | tranquilizer |
| mentyl isovalerate | tranquilizer |
| mesoridazine | tranquilizer |
| molindone | tranquilizer |
| monoamine oxidase inhibitors | tranquilizer |
| moperone | tranquilizer |
| mosapramine | tranquilizer |
| penfluridol | tranquilizer |
| perazine | tranquilizer |
| periciazine | tranquilizer |
| perphenazine | tranquilizer |
| phenothiazine | tranquilizer |
| pimozide | tranquilizer |
| pipamperone | tranquilizer |
| pipotiazine | tranquilizer |
| pregabalin | tranquilizer |
| prochlorperazine | tranquilizer |
| promazine | tranquilizer |
| promethazine | tranquilizer |
| propofol | tranquilizer |
| prothipendyl | tranquilizer |
| racetam | tranquilizer |
| selank | tranquilizer |
| selective serotonin reuptake inhibitors | tranquilizer |
| serotonin-norepinephrine reuptake inhibitor | tranquilizer |
| sulpiride | tranquilizer |
| sultopride | tranquilizer |
| sympatholytic | tranquilizer |
| thioproperazine | tranquilizer |
| thioridazine | tranquilizer |
| thiothixene | tranquilizer |
| thioxanthene | tranquilizer |
| timiperone | tranquilizer |
| tricyclic | tranquilizer |
| trifluoperazine | tranquilizer |
| triflupromazine | tranquilizer |
| veralipride | tranquilizer |
| zuclopenthixol | tranquilizer |
| 3-(4-Bromo-2,6-difluoro- benzyloxy)-5-[3-(4-pyrrolidin 1- | VEGF-related agent |
| yl- butyl)-ureido]-isothiazole-4-carboxylic acid amide | |
| hydrochloride | |
| 5-((7- Benzyloxyquinazolin-4-yl)amino)-4-fluoro-2-methyl | VEGF-related agent |
| phenol hydrochloride | |
| aflibercept | VEGF-related agent |
| AG-013958 (Pfizer Inc.) | VEGF-related agent |
| Angiogenesis inhibitor | VEGF-related agent |
| angiostatin | VEGF-related agent |
| angiozyme | VEGF-related agent |
| anti-VEGF antibody | VEGF-related agent |
| arresten | VEGF-related agent |
| AVASTIN ® | VEGF-related agent |
| axitinib | VEGF-related agent |
| bevacizumab | VEGF-related agent |
| canstatin | VEGF-related agent |
| cediranib | VEGF-related agent |
| combretastatin | VEGF-related agent |
| Combretastatin A4 Prodrug (CA4P) | VEGF-related agent |
| combstatin | VEGF-related agent |
| endogenous peptide | VEGF-related agent |
| EVIZON ™ (squalamine lactate) | VEGF-related agent |
| Fumagillin | VEGF-related agent |
| Fumagillin analogue | VEGF-related agent |
| glufanide disodium | VEGF-related agent |
| JSM6427 (Jerini AG) | VEGF-related agent |
| LUCENTIS ® | VEGF-related agent |
| MACUGEN ® | VEGF-related agent |
| multitargeted human epidermal receptor (HER) 1/2 and | VEGF-related agent |
| vascular endothelial growth factor receptor (VEGFR) 1/2 | |
| receptor family tyrosine kinases inhibitor | |
| N-(4-bromo-2-fluorophenyl)-6-methoxy-7-[(1- | VEGF-related agent |
| methylpiperidin-4-yl) methoxy]quinazol in-4-amine | |
| N-(4-bromo-2-fluorophenyl)-6-methoxy-7-[(1- | VEGF-related agent |
| methylpiperidin-4-yl) methoxy]quinazol in-4-amine | |
| N,2-dimethyl-6-(2-(l-methyl-lH-imidazol-2-yl)thieno[3,2- | VEGF-related agent |
| b]pyridin-7-yloxy)benzo[b]thiophene-3-carboxamide | |
| pan-VEGF-R-kinase inhibitor | VEGF-related agent |
| pegaptanib | VEGF-related agent |
| protein kinase inhibitor | VEGF-related agent |
| ranibizumab | VEGF-related agent |
| shark cartilage | VEGF-related agent |
| shark cartilage derivative | VEGF-related agent |
| short interfering RNA (siRNA) | VEGF-related agent |
| siRNA-based VEGFR 1 inhibitor | VEGF-related agent |
| soluble ectodomain of the VEGF receptor | VEGF-related agent |
| sorafenib | VEGF-related agent |
| synthetic peptide | VEGF-related agent |
| thalidomide | VEGF-related agent |
| thalidomide derivative | VEGF-related agent |
| thrombospondin | VEGF-related agent |
| tivozanib | VEGF-related agent |
| toll-like receptor agonist | VEGF-related agent |
| tumstatin | VEGF-related agent |
| tyrosine kinase inhibitor of the RET/PTC oncogenic kinase | VEGF-related agent |
| vatalanib | VEGF-related agent |
| VEGF agonist | VEGF-related agent |
| VEGF antagonist | VEGF-related agent |
| VEGF nucleic acid ligand | VEGF-related agent |
| VEGF therapeutic agent | VEGF-related agent |
| VEGF-R1 inhibitor | VEGF-related agent |
| VEGF-R2 inhibitor | VEGF-related agent |
| VEGFR2-selective monoclonal antibody | VEGF-related agent |
| VEGF-Trap | VEGF-related agent |
| β2-glycoprotein 1 | VEGF-related agent |

### Processed Silk as a Therapeutic Agent

In some embodiments, SBPs that consist of or include processed silk are used as therapeutic agents, wherein processed silk is an active therapeutic component. The processed silk may include, but is not limited to one or more of silk fibroin fragments of silk fibroin, chemically altered silk fibroin, and mutant silk fibroin. Therapeutic applications including such SBPs may include any of those taught in International Publication Number WO2017200659, Aykac et al. (2017) Gene s0378-1119(17)30865-8; and Abdel-Naby (2017) PLoS One 12(11):e0188154, the contents of each of which are herein incorporated by reference in their entirety. Processed silk may be administered as a therapeutic agent for treatment of a localized indication or for treatment of an indication further from the SBP application site. In some embodiments, therapeutic agents are combinations of processed silk and some other active component. In some embodiments, therapeutic agent activity requires cleavage or dissociation from silk. Therapeutic agents may include silk fibroin and/or chemically modified silk fibroin. In some embodiments, such therapeutic agents may be used to treat burn injury, inflammation, wound healing, or corneal injury. These and other treatments may be carried out according to any of the methods described in International Publication Number WO2017200659; United States Publication Number US20140235554; Aykac et al. (2017) Gene s0378-1119(17)30868-30865; or Abdel-Naby (2017) PLoS One 12(11):e0188154, the contents of each of which are herein incorporated by reference in their entirety. In some embodiments, SBPs are silk fibroin solutions used to facilitate wound healing, as described in Park et al. (2017) Acta Biomater 67:183-195, the contents of which are herein incorporated by reference in their entirety. These SBPs may enhance wound healing via a nuclear factor kappa enhancer binding protein (NF-κB) signaling pathway. In some embodiments, SBPs are therapeutic agents used to facilitate delivery and/or release of therapeutic agent payloads. Such therapeutic agents and/or methods of use may include, but are not limited to, any of those described in International Publication Number WO2017139684, the contents of which are herein incorporated by reference in their entirety.

### Biological Agents

In some embodiments, therapeutic agents include biological agents (also referred to as “biologics” or “biologicals”). As used herein, a “biological agent” refers to a therapeutic substance that is or is derived from an organism or virus. Examples of biological agents include, but are not limited to, proteins, organic polymers and macromolecules, carbohydrates, complex carbohydrates, nucleic acids, cells, tissues, organs, organisms, DNA, RNA, oligonucleotides, genes, and lipids. Biological agents may include processed silk. In some embodiments, biological agents may include any of the biologicals and compounds associated with specific categories of biological agents listed in Table 3, above. In some embodiments, biological agents may include any of those taught in International Publication Numbers WO2010123945 or WO2017123383, the contents of each of which are herein incorporated by reference in their entirety.

In some embodiments, SBPs may be used to deliver or administer biological agents. In some embodiments, delivery may include controlled release of one or more biological agents. Delivery may be carried out in vivo. In some embodiments, delivery is in vitro. Processed silk may be used to facilitate delivery and/or maintain stability of biological agents.

### Antibodies

In some embodiments, SBPs described herein are formulated with one or more antibodies. As used herein, the term “antibody” refers to a class of immune proteins that bind to specific target antigens or epitopes. Herein, “antibody” is used in the broadest sense and embraces various natural and derivative formats that include, but are not limited to monoclonal antibodies, polyclonal antibodies, multispecific antibodies (e.g., bispecific antibodies that bind to two different epitopes), antibody conjugates (e.g., antibodies conjugates with therapeutic agents, cytotoxic agents, or detectable labels), antibody variants [e.g., antibody mimetics, chimeric antibodies (e.g., having components from two or more antibody types or species), and synthetic variants], and antibody fragments. Antibodies are typically amino acid-based but may include post-translational or synthetic modifications. In some embodiments, SBPs may be used to facilitate antibody delivery, as taught in International Publication Number WO2017139684 and Guziewicz et al. (2011) Biomaterials 32(10):2642-2650, the contents of each of which are herein incorporated by reference in their entirety. In some embodiments, SBPs may be used to improve antibody stability.

In some embodiments, antibodies are VEGF antagonist or agonists. Non-limiting examples of monoclonal antibody therapeutic agents include canakinumab, palivizumab, panitumumab, inflectra, adalimumab-atto, alemtuzumab, nivolumab, ustekinumab, alefacept, ixekizumab, obiltoxaxamab, golimumab, pembrolizumab, atezolizumab, tocilizumab, basiliximab, abciximab, denosumab, omalizumab, belimumab, efalizumab, natalizumab, ustekinumab, trastuzumab, bezlotoxumab, adalimumab, rituximab, daclizumab, secukinumab, cetuximab, reslizumab, olaratumab, ipilimumab, ixekizumab, certolizumab pegol, and daclizumab. In some embodiments, antibodies may include, but are not limited to, any of those listed in Table 3, above.

### Antigens

In some embodiments, SBPs include therapeutic agents that are antigens. As used herein, the term “antigen” refers to any substance capable of provoking an immune response. In some embodiments, antigens include processed silk. In some embodiments, antigens include any of those presented in Table 3, above. In some embodiments, SBPs may be used to facilitate antigen delivery. In some embodiments, SBPs may stabilize included antigens. In some embodiments, SBPs are or are included in vaccines. Vaccines that include processed silk and methods of using such vaccines may include any of those taught in United States Publication Number US20170258889 or in Zhang et al. (2012) PNAS 109(30):11981-6 (retracted), the contents of each of which are herein incorporated by reference in their entirety. In some embodiments, formulation of an antigen with processed silk may be used to facilitate the delivery of said antigen in a vaccine, as taught in Zhang et al. (2012) PNAS 109(30):11981-6 (retracted).

### Carbohydrates

In some embodiments, therapeutic agents include carbohydrates. As used herein, the term “carbohydrate” refers to any members of a class of organic compounds that typically have carbon, oxygen, and hydrogen atoms and include, but are not limited to, simple and complex sugars. In some embodiments, carbohydrates may be monosaccharides or derivatives of a monosaccharides (e.g., ribose, glucose, fructose, galactose, mannose, abequose, arabinose, fucose, rhamnose, xylose, glucuronic acid, galactosamine, glucosamine, N-acetylgalactosamine, N-acetylglucosamine, iduronic acid, muramic acid, sialic acid, N-acetylmuramic acid, and N-acetylneuraminic acid). In some embodiments, carbohydrates may include disaccharides (e.g., sucrose, lactose, maltose, trehalose, and cellobiose). In some embodiments, carbohydrates are oligosaccharides or polysaccharides. In some embodiments, incorporation of carbohydrates may be used to stabilize SBPs. Such methods of use may include any of those taught in Li et al. (2017) Biomacromolecules 18(9):2900-5, the contents of which are herein incorporated by reference in their entirety. In some embodiments, carbohydrates may include, but are not limited to, any of those listed in Table 3, above.

### Cells and Tissues

In some embodiments, therapeutic agents include cells, tissues, organs, and/or organisms. In some embodiments, such agents are used for direct treatment. In other embodiments, cell- or tissue-based therapeutic agents are incorporated into SBPs to prepare model systems. Such methods may include any of those taught in International Publication Number WO2017189832; Chen et al. (2017) PLoS One, 12(11):e0187880; or Chen et al. (2017) Stem Cell Research and Therapy 8:260, the contents of each of which are herein incorporated by reference in their entirety. In some embodiments, incorporated cells are stem cells (e.g., see International Publication Number WO2017189832; Chendang et al. (2017) J Biomaterials and Tissue Engineering 7:858-862; Xiao et al. (2017) Oncotarget 8(49):86471-89487; Ciocci et al. (2017) Int J Biol Macromol S0141-8130(17):32839-8; Li et al. (2017) Stem Cell Res Ther 8(1):256; or Ruan et al. (2017) Biomed Pharmacother 97:600-6, the contents of each of which are herein incorporated by reference in their entirety). Examples of cell- or tissue-based therapeutic agents include, but are not limited to, human corneal stromal stem cells, human corneal epithelial cells, chicken dorsal root ganglions, bone mesenchymal stem cells, limbal epithelial stem cells, cardiac mesenchymal stem cells, adipose tissue-derived mesenchymal stem cells, periodontal ligament stem cells, human small intestinal enteroids, oral keratinocytes, fibroblasts, transfected fibroblasts, any 2-dimensional tissue, and any 3-dimensional tissue, T cells, embryonic stem cells, neural stem cells, mesenchymal stem cells, and hematopoietic stem cells. In some embodiments, cells used as therapeutic agents may include, but are not limited to, any of those listed in Table 3, above.

In some embodiments, therapeutic agents include bacteria or other microorganisms. Such therapeutic agents may be used to alter a microbiome. Examples of bacteria or other microorganisms that may be used as therapeutic agents in SBPs include any of those described in U.S. Pat. Nos. 9,688,967 and 9,688,967; US Publication Numbers US20170136073, US20170128499, US20160206666, US20170067065, and US20170014457; and International Publication Numbers WO2017123676, WO2017123675, WO2017123610, WO2017123592, WO2017123418, WO2016210384, WO2017075485, WO2017023818, WO2016210373, WO2017040719, WO2016210378, and WO2016106343, the contents of each of which are herein incorporated by reference in their entirety.

### Cytokines

In some embodiments, therapeutic agents include cytokines. As used herein, the term “cytokine” refers to a class of biological signaling molecules produced by cells that regulate cellular activity in surrounding or distant cells. In some embodiments, the cytokine is a lymphokine, monokine, growth factor, colony-stimulating factor (CSF), transforming growth factor (TGF), tumor necrosis factor (TNF), chemokine, and/or interleukin. Examples of cytokines include, but are not limited to, brain-derived neurotrophic factor (BDNF), cardiotrophin-like cytokine factor 1 (CLCF1), ciliary neurotrophic factor (CNTF), cardiotrophin 1 (CTF1), epidermal growth factor (EGF), erythropoietin (EPO), fibroblast growth factor acidic (FGFa), fibroblast growth factor basic (FGFb), granulocyte colony stimulating factor (G-CSF), growth hormone, granulocyte-macrophage colony stimulating factor 2 (GM-CSF), interferon-α1, interleukin (IL)-1 (IL-1), IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-27, interleukin-1 receptor antagonist (IL-1RA), keratinocyte growth factor 1 and 2 (KGF), kit ligand/stem cell factor (KITLG), leptin (LEP), leukemia inhibitory factor (LIF), nerve growth factor (NGF), oncostatin M (OSM), platelet derived growth factors, prolactin (PRL), thrombopoietin (THPO), transforming growth factor (TGF) α (TGFα), TGFβ, tumor necrosis factor α (TNFα), vascular endothelial growth factor (VEGF), tissue inhibitor of metalloproteinase (TIMP), matrix metalloproteinase (MMP), any of the interferons, any of the interleukins, any of the lymphokines, any of the cell signal molecules, and any structural or functional molecule thereof. In some embodiments, cytokines may include, but are not limited to, any of those listed in Table 3, above.

### Lipids

In some embodiments, therapeutic agents include lipids. As used herein, the term “lipid” refers to members of a class of organic compounds that include fatty acids and various derivatives of fatty acids that are soluble in organic solvents, but not in water. Examples of lipids include, but are not limited to, fats triglycerides, oils, waxes, sterols (e.g. cholesterol, ergosterol, hopanoids, hydroxysteroids, phytosterol, and steroids), stearin, palmitin, triolein, fat-soluble vitamins (e.g., vitamins A, D, E, and K), monoglycerides (e.g. monolaurin, glycerol monostearate, and glyceryl hydroxystearate), diglycerides (e.g. diacylglycerol), phospholipids, glycerophospholipids (e.g., phosphatidic acid, phosphatidylethanolamine, phosphatidylcholine, phosphatidylserine, phosphoinositides), sphingolipids (e.g., sphingomyelin), and phosphosphingolipids. In some embodiments, lipids may include, but are not limited to, any of those listed (e.g., fats and fatty acids) in Table 3, above.

### Macromolecules

In some embodiments, therapeutic agents include macromolecules, cells, tissues, organs, and/or organisms. Examples of macromolecules include, but are not limited to, proteins, polymers, carbohydrates, complex carbohydrates, lipids, nucleic acids, oligonucleotides, and genes. Macromolecules may be expressed (e.g. expression in Escherichia coli) or they may be chemically synthesized (e.g. solid phase synthesis, and/or polymer forming chain reactions).

### Microorganism and Microbiomes

In some embodiments, therapeutic agents include cellular therapeutics, such as bacteria and/or other microorganisms. In some embodiments, SBPs may be used to deliver cellular therapeutics (e.g., bacteria and/or other microorganisms) to alter or improve the microbiome of a subject or patient. In some embodiments, bacteria and/or other microorganisms used as therapeutic agents may include, but are not limited to, any of those described in U.S. Pat. No. 9,688,967, or 9,688,967; in US Publication Numbers US20170136073, US20170128499, US20160206666, US20170067065, or US20170014457; or in International Publication Numbers WO2017123676, WO2017123675, WO2017123610, WO2017123592, WO2017123418, WO2016210384, WO2017075485, WO2017023818, WO2016210373, WO2017040719, WO2016210378, or WO2016106343, the contents of each of which are herein incorporated by reference in their entirety.

In some embodiments, bacteria and/or other microorganisms may be used for the treatment of diseases associated with hyperammonemia, e.g., as described in the U.S. Pat. No. 9,688,967, and the WIPO Publication Numbers WO2016200614 and WO2017087580, the contents of each of which are herein incorporated by reference in their entirety. In some embodiments, said bacteria and/or microorganisms are formulated as a part of SBPs. In some embodiments, the bacteria and/or microorganisms may be supported during delivery using SBPs. In some embodiments, bacteria and/or other microorganisms used as therapeutic agents may be engineered, e.g., by any method described in the U.S. Pat. No. 9,688,967 or 9,487,764; or in International Publication Numbers WO2016200614 and WO2017087580, the contents of each of which are herein incorporated by reference in their entirety.

In some embodiments, bacteria and/or other microorganisms may be used for the treatment of diseases or disorders described in the US Publication Number US20170136073, the contents of which are herein incorporated by reference in their entirety. Such bacteria and/or other microorganisms may be engineered, e.g., using any of the methods described in US Publication Number US20170136073. In some embodiments, bacteria and/or other microorganisms may be used for the treatment of diseases that benefit from reduced gut inflammation and/or tightened gut mucosal barrier, e.g., as described in US Publication Numbers US20170128499, US20160206666, and US20170067065, the contents of each of which are herein incorporated by reference in their entirety. In some embodiments, bacteria and/or other microorganisms formulated as part of SBPs may be used to reduce hyperphenylalaninemia, e.g., as described in the US Publication Numbers US20170014457, and US20170067065, the contents of each of which are herein incorporated by reference in their entirety. In some embodiments, bacteria and/or other microorganisms formulated as part of SBPs may be used to treat diseases and disorders associated with amino acid metabolism as described in WIPO Publication Number WO2017123676, the contents of which are herein incorporated by reference in their entirety. In some embodiments, bacteria and/or other microorganisms formulated as part of SBPs may be used to produce immune modulators and anti-cancer therapeutics in tumor cells as described in WIPO Publication Number WO2017123675, the contents of which are herein incorporated by reference in their entirety. In some embodiments, bacteria and/or other microorganisms formulated as part of SBPs of the present disclosure may be used to detoxify deleterious molecules as described in WIPO Publication Number WO2017123610, the contents of which are herein incorporated by reference in their entirety. In some embodiments, bacteria and/or other microorganisms formulated as part of SBPs may be used to treat disorders associated with bile salts as described in WIPO Publication Number WO2017123592, the contents of which are herein incorporated by reference in their entirety. In some embodiments, bacteria and/or other microorganisms formulated as a part of SBPs may be used to treat metabolic diseases as described in WIPO Publication Numbers WO2017123418, and WO2016210384, the contents of each of which are herein incorporated by reference in their entirety. In some embodiments, bacteria and/or other microorganisms formulated as a part of SBPs may be used to treat disorders in which trimethylamine (Tma) is detrimental as described in WIPO Publication Number WO2017075485, the contents of which are herein incorporated by reference in their entirety. In some embodiments, bacteria and/or other microorganisms formulated as a part of SBPs may be used for biosafety and/or pharmaceutical compositions as described in WIPO Publication Number WO2016210373, the contents of which are herein incorporated by reference in their entirety. In some embodiments, bacteria and/or other microorganisms formulated as a part of SBPs may be used to treat disorders in which oxalate is detrimental as described in the WIPO Publication Number WO2017040719, the contents of which are herein incorporated by reference in their entirety. In some embodiments, bacteria and/or other microorganisms formulated as a part of SBPs may comprise circuits for multi-layered control of gene expression, e.g., when used as described in WIPO Publication Number WO2016210378, the contents of which are herein incorporated by reference in their entirety.

In some embodiments, bacteria and/or other microorganisms formulated as a part of SBPs may be probiotic organisms for diagnosis, monitoring, and treatment of inflammatory bowel disease, e.g., when used as described in WIPO Publication Number WO2016106343, the contents of which are herein incorporated by reference in their entirety.

In some embodiments, SBPs described herein maintain and/or improve the stability of bacteria and/or other microorganisms. The maintenance and/or improvement of stability may be determined by comparing stability with SBP compositions to stability with compositions lacking SBPs or to standard compositions in the art. Maintenance and/or improvement of stability may be found or appreciated where superior or durational benefits are observed with SBPs. In some embodiments, SBPs maintain and/or improve the stability of bacteria and/or other microorganisms that can be used in bacterial or microbial therapy.

In some embodiments, bacteria and/or other microorganisms may be used as biopesticides. As used herein, the term “biopesticide” refers to a composition with a bacteria, microorganisms, or biological cargo used to harm, kill, or prevent the spread of pests. Biopesticides have been used in agricultural development, as described in U.S. Pat. No. 6,417,163, the contents of which are herein incorporated by reference in their entirety. In some embodiments, SBPs that include bacteria, microorganisms, and/or microbiomes, may be used as biopesticides to support agricultural applications.

In some embodiments, bacteria and/or other microorganisms formulated as a part of SBPs may include one or more of the following microbes. The names of the microbes provided herein may optionally include the strain name. Abiotrophia, Abiotrophia defectiva, Acetanaerobacterium, Acetanaerobacterium elongatum, Acetivibrio, Acelivibrio bacterium, Acetobacterium, Acetobacterium woodii, Acholeplasma, Acidaminococcus, Acidaminococcus fermentans, Acidianus, Acidianus brierleyi, Acidovorax, Acinetobacter, Acinetobacter guillouiae, Acinetobacter junii, Actinobacillus, Actinobacillus M1933/96/1, Actinomyces, Actinomyces ICM34, Actinomyces ICM41, Actinomyces ICM54, Actinomyces lingnae, Actinomyces odontolyticus, Actinomyces oral, Actinomyces ph3, Adlercreutzia, Adlercreutzia equolifaciens, Adlercreutzia intestinal, Aerococcus, Aeromonas, Aeromonas 165C, Aeromonas hydrophila, Aeromonas RC50, Aeropyrum, Aeropyrum pernix, agglomerans, Aggregatibacter, Agreia, Agreia bicolorata, Agromonas, Agromonas CS30, Akkermansia, Akkermansia muciniphila, Alistipes, Alistipes ANH, Alistipes AP1 1, Alistipes bacterium, Alistipes CCUG, Alistipes DJF B85, Alistipes DSM, Alistipes EBA6-25cl2, Alistipes finegoldii, Alistipes indistinctus, Alistipes JC136, Alistipes NML05A004, Alistipes onderdonkii, Alistipes putredinis, Alistipes RMA, Alistipes senegalensis, Alistipes shahii, Alistipes smarlab, Alkalibaculum, Alkaliflexus, Allisonella, Allisonella histaminiformans, Alloscardovia, Alloscardovia omnicolens, Anaerofilum, Anaerofustis, Anaerofustis stercorihominis, Anaeroplasma, Anaerostipes, Anaerostipes 08964, Anaerostipes 494a, Anaerostipes 5_1_63FAA, Anaerostipes AIP, Anaerostipes bacterium, Anaerostipes butyraticus, Anaerostipes caccae, Anaerostipes hadrum, Anaerostipes IE4, Anaerostipes indolis, Anaerostipes ly-2, Anaerotruncus, Anaerotruncus colihominis, Anaerotruncus NML, Aquincola, Arcobacter, Arthrobacter, Arthrobacter FVl-1, Asaccharobacter, Asaccharobacter celatus, Asteroleplasma, Atopobacter, Atopobacter phocae, Atopobium, Atopobium parvulum, Atopobium rimae, Bacteriovorax, Bacteroides Bacteroides 31SF18, Bacteroides 326-8, Bacteroides 35AE31, Bacteroides 35AE37, Bacteroides 35BE34, Bacteroides 4072, Bacteroides 7853, Bacteroides acidifaciens, Bacteroides API, Bacteroides AR20, Bacteroides AR29, Bacteroides B2, Bacteroides bacterium, Bacteroides barnesiae, Bacteroides BLBE-6, Bacteroides BV-1, Bacteroides caccae, Bacteroides CannelCatfish9 Bacteroides cellulosilyticus, Bacteroides chinchillae, Bacteroides CIP 103040, Bacteroides clarus, Bacteroides coprocola, Bacteroides coprophilus, Bacteroides D8, Bacteroides DJF_B097, Bacteroides dnLKV2, Bacteroides dnLKV7, Bacteroides dnLKV9, Bacteroides dorei, Bacteroides EBA5-17, Bacteroides eggerthii, Bacteroides enrichment, Bacteroides F-4, Bacteroides faecichinchillae, Bacteroides faecis, Bacteroides fecal, Bacteroides finegoldii, Bacteroides fragilis, Bacteroides gallinarum, Bacteroides helcogenes, Bacteroides icl292, Bacteroides intestinalis, Bacteroides massiliensis, Bacteroides mpnisolate, Bacteroides NB-8, Bacteroides new, Bacteroides NLAE-zl-c204, Bacteroides NLAE-zl-c205, Bacteroides NLAE-zl-c206, Bacteroides NLAE-zl-c207, Bacteroides NLAE-zl-c211, Bacteroides NLAE-zl-c218, Bacteroides NLAE-zl-c257, Bacteroides NLAE-zl-c260, Bacteroides NLAE-zl-c261, Bacteroides NLAE-zl-c263, Bacteroides NLAE-zl-c308, Bacteroides NLAE-zl-c315, Bacteroides NLAE-zl-c322, Bacteroides NLAE-zl-c324, Bacteroides NLAE-zl-c331, Bacteroides NLAE-zl-c339, Bacteroides NLAE-zl-c36, Bacteroides NLAE-zl-c367, Bacteroides NLAE-zl-c375, Bacteroides NLAE-zl-c376, Bacteroides NLAE-zl-c380, Bacteroides NLAE-zl-c391, Bacteroides NLAE-zl-c459, Bacteroides NLAE-zl-c484, Bacteroides NLAE-zl-c501, Bacteroides NLAE-zl-c504, Bacteroides NLAE-zl-c515, Bacteroides NLAE-zl-c519, Bacteroides NLAE-zl-c532, Bacteroides NLAE-zl-c557, Bacteroides NLAE-zl-c57, Bacteroides NLAE-zl-c574, Bacteroides NLAE-zl-c592, Bacteroides NLAE-zl-cl3, Bacteroides NLAE-zl-cl58, Bacteroides NLAE-zl-c59, Bacteroides NLAE-zl-cl61, Bacteroides NLAE-zl-cl63, Bacteroides NLAE-zl-cl67, Bacteroides NLAE-zl-cl72, Bacteroides NLAE-zl-cl8, Bacteroides NLAE-zl-cl82, Bacteroides NLAE-zl-cl90, Bacteroides NLAE-zl-cl98, Bacteroides NLAE-zl-g209, Bacteroides NLAE-zl-g212, Bacteroides NLAE-zl-g213, Bacteroides NLAE-zl-g218, Bacteroides NLAE-zl-g221, Bacteroides NLAE-zl-g228, Bacteroides NLAE-zl-g234, Bacteroides NLAE-zl-g237, Bacteroides NLAE-zl-g24, Bacteroides NLAE-zl-g245, Bacteroides NLAE-zl-g257, Bacteroides NLAE-zl-g27, Bacteroides NLAE-zl-g285, Bacteroides NLAE-zl-g288, Bacteroides NLAE-zl-g295, Bacteroides NLAE-zl-g296, Bacteroides NLAE-zl-g303, Bacteroides NLAE-zl-g310, Bacteroides NLAE-zl-g312, Bacteroides NLAE-zl-g327, Bacteroides NLAE-zl-g329, Bacteroides NLAE-zl-g336, Bacteroides NLAE-zl-g338, Bacteroides NLAE-zl-g347, Bacteroides NLAE-zl-g356 Bacteroides NLAE-zl-g373, Bacteroides NLAE-zl-g376, Bacteroides NLAE-zl-g380, Bacteroides NLAE-zl-g382, Bacteroides NLAE-zl-g385, Bacteroides NLAE-zl-g4, Bacteroides NLAE-zl-g422, Bacteroides NLAE-zl-g437, Bacteroides NLAE-zl-g454, Bacteroides NLAE-zl-g455, Bacteroides NLAE-zl-g456, Bacteroides NLAE-zl-g458, Bacteroides NLAE-zl-g459, Bacteroides NLAE-zl-g46, Bacteroides NLAE-zl-g461, Bacteroides NLAE-zl-g475, Bacteroides NLAE-zl-g481, Bacteroides NLAE-zl-g484, Bacteroides NLAE-zl-g5, Bacteroides NLAE-zl-g502 Bacteroides NLAE-zl-g515, Bacteroides NLAE-zl-g518, Bacteroides NLAE-zl-g521, Bacteroides NLAE-zl-g54, Bacteroides NLAE-zl-g6, Bacteroides NLAE-zl-g8, Bacteroides NLAE-zl-g80, Bacteroides NLAE-zl-g98, Bacteroides NLAE-zl-gl 17, Bacteroides NLAE-zl-gl05, Bacteroides NLAE-zl-gl27, Bacteroides NLAE-zl-gl36, Bacteroides NLAE-zl-gl43, Bacteroides NLAE-zl-gl57, Bacteroides NLAE-zl-gl67, Bacteroides NLAE-zl-gl71, Bacteroides NLAE-zl-gl87, Bacteroides NLAE-zl-gl94, Bacteroides NLAE-zl-gl95, Bacteroides NLAE-zl-gl99, Bacteroides NLAE-zl-h207, Bacteroides NLAE-zl-h22, Bacteroides NLAE-zl-h250, Bacteroides NLAE-zl-h251, Bacteroides NLAE-zl-h28, Bacteroides NLAE-zl-h313, Bacteroides NLAE-zl-h319, Bacteroides NLAE-zl-h321, Bacteroides NLAE-zl-h328, Bacteroides NLAE-zl-h334, Bacteroides NLAE-zl-h390, Bacteroides NLAE-zl-h391, Bacteroides NLAE-zl-h414, Bacteroides NLAE-zl-h416, Bacteroides NLAE-zl-h419, Bacteroides NLAE-zl-h429, Bacteroides NLAE-zl-h439, Bacteroides NLAE-zl-h444, Bacteroides NLAE-zl-h45, Bacteroides NLAE-zl-h46, Bacteroides NLAE-zl-h462, Bacteroides NLAE-zl-h463, Bacteroides NLAE-zl-h465, Bacteroides NLAE-zl-h468, Bacteroides NLAE-zl-h471, Bacteroides NLAE-zl-h472, Bacteroides NLAE-zl-h474, Bacteroides NLAE-zl-h479, Bacteroides NLAE-zl-h482, Bacteroides NLAE-zl-h49, Bacteroides NLAE-zl-h493, Bacteroides NLAE-zl-h496, Bacteroides NLAE-zl-h497, Bacteroides NLAE-zl-h499, Bacteroides NLAE-zl-h50, Bacteroides NLAE-zl-h531, Bacteroides NLAE-zl-h535, Bacteroides NLAE-zl-h8, Bacteroides NLAE-zl-hl20, Bacteroides NLAE-zl-hl5, Bacteroides NLAE-zl-hl62, Bacteroides NLAE-zl-hl7, Bacteroides NLAE-zl-hl74, Bacteroides NLAE-zl-h18, Bacteroides NLAE-zl-hl88, Bacteroides NLAE-zl-hl92, Bacteroides NLAE-zl-hl94, Bacteroides NLAE-zl-hl95, Bacteroides NLAE-zl-p208, Bacteroides NLAE-zl-p213, Bacteroides NLAE-zl-p228, Bacteroides NLAE-zl-p233, Bacteroides NLAE-zl-p267, Bacteroides NLAE-zl-p278, Bacteroides NLAE-zl-p282, Bacteroides NLAE-zl-p286, Bacteroides NLAE-zl-p295, Bacteroides NLAE-zl-p299, Bacteroides NLAE-zl-p301, Bacteroides NLAE-zl-p302, Bacteroides NLAE-zl-p304, Bacteroides NLAE-zl-p317, Bacteroides NLAE-zl-p319, Bacteroides NLAE-zl-p32, Bacteroides NLAE-zl-p332, Bacteroides NLAE-zl-p349, Bacteroides NLAE-zl-p35, Bacteroides NLAE-zl-p356, Bacteroides NLAE-zl-p370, Bacteroides NLAE-zl-p371, Bacteroides NLAE-zl-p376, Bacteroides NLAE-zl-p395, Bacteroides NLAE-zl-p402, Bacteroides NLAE-zl-p403, Bacteroides NLAE-zl-p409, Bacteroides NLAE-zl-p412, Bacteroides NLAE-zl-p436, Bacteroides NLAE-zl-p438, Bacteroides NLAE-zl-p440, Bacteroides NLAE-zl-p447, Bacteroides NLAE-zl-p448, Bacteroides NLAE-zl-p451, Bacteroides NLAE-zl-p476, Bacteroides NLAE-zl-p478, Bacteroides NLAE-zl-p483, Bacteroides NLAE-zl-p489, Bacteroides NLAE-zl-p493, Bacteroides NLAE-zl-p557, Bacteroides NLAE-zl-p559, Bacteroides NLAE-zl-p564, Bacteroides NLAE-zl-p565, Bacteroides NLAE-zl-p572, Bacteroides NLAE-zl-p573, Bacteroides NLAE-zl-p576, Bacteroides NLAE-zl-p591, Bacteroides NLAE-zl-p592, Bacteroides NLAE-zl-p631, Bacteroides NLAE-zl-p633, Bacteroides NLAE-zl-p696, Bacteroides NLAE-zl-p7, Bacteroides NLAE-zl-p720, Bacteroides NLAE-zl-p730, Bacteroides NLAE-zl-p736, Bacteroides NLAE-zl-p737, Bacteroides NLAE-zl-p754, Bacteroides NLAE-zl-p759, Bacteroides NLAE-zl-p774, Bacteroides NLAE-zl-p828, Bacteroides NLAE-zl-p854, Bacteroides NLAE-zl-p860, Bacteroides NLAE-zl-p886, Bacteroides NLAE-zl-p887, Bacteroides NLAE-zl-p900 Bacteroides NLAE-zl-p909, Bacteroides NLAE-zl-p913, Bacteroides NLAE-zl-p916, Bacteroides NLAE-zl-p920, Bacteroides NLAE-zl-p96, Bacteroides NLAE-zl-p104, Bacteroides NLAE-zl-pl05, Bacteroides NLAE-zl-pl08, Bacteroides NLAE-zl-pl32, Bacteroides NLAE-zl-pl33, Bacteroides NLAE-zl-pl51, Bacteroides NLAE-zl-pl57, Bacteroides NLAE-zl-pl66, Bacteroides NLAE-zl-pl67, Bacteroides NLAE-zl-p171, Bacteroides NLAE-zl-pl78, Bacteroides NLAE-zl-pl87, Bacteroides NLAE-zl-pl91, Bacteroides NLAE-zl-pl96, Bacteroides nordii, Bacteroides oleiciplenus, Bacteroides ovatus, Bacteroides paurosaccharolyticus, Bacteroides plebeius, Bacteroides R6, Bacteroides rodentium, Bacteroides S-17, Bacteroides S-18, Bacteroides salyersiae, Bacteroides SLCl-38, Bacteroides smarlab, Bacteroides smarlab, Bacteroides stercorirosoris, Bacteroides stercoris, Bacteroides str, Bacteroides thetaiotaomicron, Bacteroides TP-5, Bacteroides uniormis, Bacteroides vulgatus, Bacteroides WA1, Bacteroides WH2, Bacteroides WH302, Bacteroides WH305, Bacteroides X077B42, Bacteroides XB12B, Bacteroides XB44A, Bacteroides xylanisolvens, Barnesiella, Barnesiella intestinihominis, Barnesiella NSBI, Barnesailla viscericola, Bavariicoccus, Bdellovibrio, Bdellovabrio oral, Bergeriella, Bifidobacterium, Bifidobacterium 103, Bifidobacterium 108, Bifidobacterium 113, Bifidobacterium 120, Bifidobacterium 138, Bifidobacterium 33, Bifdobacterium Acbbto5, Bifidobacterium adolescentis, Bifidobacterium Amsbbtl2, Bifidobacterium angulatum, Bifidobacterium animalis, Bifidobacterium bacterium, Bifidobacterium bifidum, Bifdobacterium Bisn6, Bifdobacterium Bma6, Bifdobacterium breve, Bifdobacterium catenulatum, Bifidobacterium choerinum, Bifdobacterium coryneforme, Bifidobacterium dentium, Bifidobacterium DJF_WC44, Bifdobacterium F-10, Bifidobacterium F-11, Bifdobacterium group, Bifidobacterium hl2, Bifdobacterium HMLN1, Bifdobacterium HMLN12, Bifidobacterium HMLN5, Bifidobacterium iarfr2341d, Bifdobacterium iarfr642d48, Bifidobacterium icl332, Bifidobacterium indicum, Bifdobacterium kashiwanohense, Bifidobacterium LISLUCIII-2, Bifidobacterium longum, Bifidobacterium M45, Bifidobacterium merycicum, Bifdobacterium minimum, Bifidobacterium MX5B, Bifidobacterium oral, Bifidobacterium PG12A, Bifidobacterium PL1, Bifidobacterium pseudocatenulatum, Bifidobacterium pseudolongum, Bifidobacterium pullorum, Bifdobacterium ruminantium, Bifidobacterium S-10, Bifidobacterium saeculare, Bifidobacterium saguini, Bifidobacterium scardovii, Bifidobacterium simiae, Bifidobacterium SLPYG-1, Bifidobacterium stercoris, Bifidobacterium TM-7, Bifidobacterium Trm9, Bilophila, Bilophila NLAE-zl-h528, Bilophila wadsworthia, Blautia, Blautia bacterium, Blautia CE2, Blautia CE6, Blautia coccoides, Blautia DJF_VR52, Blautia DJF_VR67, Blautia DJT_VR70kl, Blautia formate, Blautia glucerasea, Blautia hansenii, Blautia icl272, Blautia IE5, Blautia K-1, Blautia luti, Blautia M-1, Blautia mpnisolate, Blautia NLAE-zl-c25, Blautia NLAE-zl-c259, Blautia NLAE-zl-c51, Blautia NLAE-zl-c520, Blautia NLAE-zl-c542, Blautia NLAE-zl-c544, Blautia NLAE-zl-h27, Blautia NLAE-zl-h316, Blautia NLAE-zl-h317, Blautia obeum, Blautia producta, Blautia productus, Blautia schinkii, Blautia Ser5, Blautia Ser8, Blautia WAL, Blauria wexlerae, Blautia YHC-4, Brenneria, Brevibacterium, Brochothrix, Brochothrix thermosphacta, Buttiauxella, Buttiauxella 57916, Buttiauxella gaviniae, Butyricicoccus, Butyricicoccus bacterium, Butyricimonas, Butyricimonas 180-3, Butyricimonas 214-4, Butyricimonas bacterium, Butyricimonas GD2, Butyricimonas synergistica, Butyricimonas virosa, Butyrivibrio, Butyrivibrio fibrisolvens, Butyrivibrio hungatei, Caldimicrobium, Caldisericum, Campylobacter, Campylobacter coli, Campylobacter hominis, Capnocytophaga, Carnobacterium, Carnobacterium alterfunditum, Caryophanon, Catenibacterium, Catenibacterium mitsuokai, Catonella, Caulobacter, Cellulophaga, Cellulosilyticum, Cetobacterium, Chelatococcus, Chlorobium, Chryseobacterium, Chryseobacterium A1005, Chryseobacterium KJ9C8, Citrobacter, Citrobacter 1, Citrobacter 191-3, Citrobacter agglomerans, Citrobacter amalonaticus, Citrobacter ascorbata, Citrobacter bacterium, Citrobacter BinzhouCLT, Citrobacter braakii, Citrobacter enrichment, Citrobacter F24, Citrobacter F96, Citrobacter farmeri, Citrobacter freundii, Citrobacter gillenii, Citrobacter HBKC_SR1, Citrobacter HD4.9, Citrobacter hormaechei, Citrobacter ka55, Citrobacter lapagei, Citrobacter LAR-1, Citrobacter ludwigii, Citrobacter MEB5, Citrobacter MS36, Citrobacter murliniae, Citrobacter NLAE-zl-c269, Citrobacter P014, Citrobacter P042bN, Citrobacter P046a, Citrobacter P073, Citrobacter SR3, Citrobacter Tl, Citrobacter tnt4, Citrobacter tnt5, Citobacter trout, Citrobacter TSA-1, Citrobacter werkmanii, Cloacibacillus, Cloacibacillus adv66, Cloacibacillus NLAE-zl-p702, Cloacibacillus NML05A017, Cloacibacterium, Collinsella, Collinsella aerofaciens, Collinsella A-1, Collinsella AUH-Julong21, Collinsella bacterium, Collinsella CCUG, Comamonas, Comamonas straminea, Comamonas testosteroni, Conexibacter, Coprobacillus, Coprobacillus bacterium, Coprobacillus cateniformis, Coprobacillus TM-40, Coprococcus, Coprococcus 14505, Coprococcus bacterium, Coprococcus catus, Coprococcus comes, Coprococcus eutactus, Coprococcus nexile, Coraliomargarita, Coraliomargarita fucoidanolyticus, Coraliomargarita marisflavi, Corynebacterium, Corynebacterium amycolatum, Corynebacterium durum, Coxiella, Cronobacter, Cronobacter dublinensis, Cronobacter sakazakii, Cronobacter turicensis, Cryptobacterium, Cryptobacterium curtum, Cupriavidus, Cupriavidus eutropha, Dechloromonas, Dechloromonas HZ, Desulfobacterum, Desulfobulbus, Desufopila, Desulfopila La4.1, Desulfovibrio, Desulfovibrio D4, Desulfovibrio desulfuricans, Desulfovibrio DSM 12803, Desulfovibrio enrichment, Desulfovibrio fairfieldensis, Desulfovibrio LNB1, Desulfovibrio piger, Dialister, Dialister E2_20, Dialister GBA27, Dialister invisus, Dialister oral, Dialister succinatiphilus, Dorea, Dorea auhjulong64, Dorea bacterium, Dorea formicigenerans, Dorea longicatena, Dorea mpnisolate, Dysgonomonas, Dysgonomonas gadei, Edwardsiella, Edwardsiella tarda, Eggerthella, Eggerthella El, Eggerthella lenta, Eggerthella MLG043, Eggerthella MVA1, Eggerthella S6-C1, Eggerthella SDG-2, Eggerthella sinensis, Eggerthella str, Enhydrobacter, Enterobacter, Enterobacter 1050, Enterobacter 112, Enterobacter 1122, Enterobacter 77000, Enterobacter 82353, Enterobacter 9C, Enterobacter ASC, Enterobacter adecarboxylata, Enterobacter aerogenes, Enterobacter agglomerans, Enterobacter AJAR-A2, Enterobacter amnigenus, Enterobacter asburiae, Enterobacter B 1(2012), Enterobacter B363, Enterobacter B509, Enterobacter bacterium, Enterobacter Badong3, Enterobacter BEC441, Enterobacter C8, Enterobacter cancerogenus, Enterobacter cloacae, Enterobacter CO, Enterobacter core2, Enterobacter cowanii, Enterobacter dc6, Enterobacter DRSBIL, Enterobacter enrichment, Enterobacter FL13-2-1, Enterobacter GIST-NKst9, Enterobacter GIST-NKstlO, Enterobacter GJl-11, Enterobacter gx-148, Enterobacter hormaechei, Enterobacter I-Bh20-21, Enterobacter ICB113, Enterobacter kobei, Enterobacter KW4, Enterobacter ludwigii, Enterobacter M10_1B, Enterobacter M1R3, Enterobacter marine, Enterobacter NCCP-167, Enterobacter of, Enterobacter oryzae, Enterobacter oxytoca, Enterobacter P101, Enterobacter SEL2, Enterobacter SI 1, Enterobacter SPh, Enterobacter SSASP5, Enterobacter terrigena, Enterobacter TNT3, Enterobacter TP2MC, Enterobacter TS4, Enterobacter TSSAS2-48, Enterobacter ZYXCA1, Enterococcus, Enterococcus 020824/02-A, Enterococcus 1275b, Enterococcus 16C, Enterococcus 48, Enterococcus 6114, Enterococcus ABRIINW-H61, Enterococcus asini, Enterococcus avium, Enterococcus azikeevi, Enterococcus bacterium, Enterococcus BBDP57, Enterococcus BPH34, Enterococcus Bt, Enterococcus canis, Enterococcus casselifavus, Enterococcus CmNA2, Enterococcus Da-20, Enterococcus devriesei, Enterococcus dispar, Enterococcus DJF_O30, Enterococcus DMB4, Enterococcus durans, Enterococcus enrichment, Enterococcus F81, Enterococcus faecalis, Enterococcus faecium, Enterococcus fcc9, Enterococcus fecal, Enterococcus flavescens, Enterococcus fluvialis, Enterococcus FR-3, Enterococcus FUA3374, Enterococcus gallinarum, Enterococcus GSC-2, Enterococcus GYPB01, Enterococcus hermanniensis, Enterococcus hirae, Enterococcus lactis, Enterococcus malodoratus, Enterococcus manure, Enterococcus marine, Enterococcus MNC1, Enterococcus moraviensis, Enterococcus M52, Enterococcus mundtii, Enterococcus NAB 15, Enterococcus NBRC, Enterococcus NLAE-zl-c434, Enterococcus NLAE-zl-g87, Enterococcus NLAE-zl-gl06, Enterococcus NLAE-zl-h339, Enterococcus NLAE-zl-h375, Enterococcus NT AE-zl-h381, Enterococcus NLAE-zl-h383, Enterococcus NLAE-zl-h405, Enterococcus NLAE-zl-p401, Enterococcus NLAE-zl-p650, Enterococcus NLAE-zl-pl 16, Enterococcus NLAE-zl-pl48, Enterococcus pseudoavium, Enterococcus R-25205, Enterococcus raffinosus, Enterococcus rottae, Enterococcus RU07, Enterococcus saccharolyticus, Enterococcus saccharominimus, Enterococcus sanguinicola, Enterococcus SCA16, Enterococcus SCA2, Enterococcus SE138, Enterococcus SF-1, Enterococcus sulfureus, Enterococcus SV6, Enterococcus te32a, Enterococcus te42a, Enterococcus te45r, Enterococcus te49a, Enterococcus te51a, Enterococcus te58r, Enterococcus te59r, Enterococcus te61r, Enterococcus te93r, Enterococcus te95a, Enterococcus tela, Enterorhabdus, Enterorhabdus caecimuris, entomophaga, Erwinia, Erwinia agglomerans, Erwinia enterica, Erwinia rhapontici, Erwinia tasmaniensis, Erysipelotrichaceae incertae sedis, Erysipelotrichaceae incertae sedis aff, Erysipelotrichaceae incertae sedis bacterium, Erysipelotrichaceae incertae sedis biforme, Erysipelotrchaceae incertae sedis C-l, Erysipelotrichaceae incertae sedis cylindroides, Erysipelotrichaceae incertae sedis GK12, Erysipelotrichaceae incertae sedis innocuum, Erysipelotrichaceae incertae sedis NLAE-zl-c332, Erysipelotrichaceae incertae sedis NLAE-zl-c340, Erysipelotrichaceae incertae sedis NLAE-zl-g420, Erysipelotrichaceae incertae sedis NLAE-zl-g425, Erysipelotrichaceae incertae sedis NLAE-zl-g440, Erysipelotrichaceae incertae sedis NLAE-zl-g463, Erysipelotrichaceae incertae sedis NIAF-zi-h340, Erysipelotrichaceae incertae sedis NLAE-zl-h354, Erysipelotrichaceae incertae sedis NLAE-zl-h379, Erysipelotrichaceae incertae sedis NLAE-zl-h380, Erysipelotrichaceae incertae sedis NLAE-zl-h385, Erysipelotrichaceae incertae sedis NLAE-zl-h410, Erysipelotrichaceae incertae sedis tortuosum, Escherichia/Shigella, Escherichia/Shigella 29(2010), Escherichia/Shigella 4091, Escherichia/Shigella 4104, Escherichia/Shigella 8gwl8, Escherichia/Shigella A94, Escherichia/Shigella albertii, Escherichia/Shigella B-1012, Escherichia/Shigella B4, Escherichia/Shigella bacterium, Escherichia/Shigella BBDP15, Escherichia/Shigella BBDP80, Escherichia/Shigella boydii, Escherichia/Shigella carotovorum, Escherichia/Shigella CERAR, Escherichia/Shigella coli, Escherichia/Shigella DBC-1, Escherichia/Shigella dc262011, Escherichia/Shigella dysenteriae, Escherichia/Shigella enrichment, Escherichia/Shigella escherichia, Escherichia/Shigella fecal, Escherichia/Shigella fergusonii, Escherichia/Shigella flexneri, Escherichia/Shigella GDR05, Escherichia/Shigella GDR07, Escherichia/Shigella H7, Escherichia/Shigella marine, Escherichia/Shigella ML2-46, Escherichia/Shigella mpnisolate, Escherichia/Shigella NA Escherichia/Shigella NLAE-zl-g330, Escherichia/Shigella NLAE-zl-g400, Escherichia/Shigella NLAE-zl-g441, Escherichia/Shigella NLAE-zl-g506, Escherichia/Shigella NLAE-zl-h204, Escherichia/Shigella NLAE-zl-h208, Escherichia/Shigella NLAE-zl-h209, Escherichia/Shigella NLAE-zl-h213, Escherichia/Shigella NLAE-zl-h214, Escherichia/Shigella NLAE-zl-h4, Escherichia/Shigella NLAE-zl-h435, Escherichia/Shigella NLAE-zl-h81, Escherichia Shigella NLAE-zl-p21, Escherichia/Shigella NLAE-zl-p235, Escherichia/Shigella NLAE-zl-p237, Escherichia/Shigella NLAE-zl-p239, Escherichia/Shigella NLAE-zl-p25, Escherichia/Shigella NLAE-zl-p252, Escherichia/Shigella NLAE-zl-p275, Escherichia/Shigella NLAE-zl-p280, Escherichia/Shigella NLAE-zl-p51, Escherichia/Shigella NLAE-zl-p53, Escherichia/Shigella NLAE-zl-p669, Escherichia/Shigella NLAE-zl-p676, Escherichia/Shigella NLAE-zl-p717, Escherichia/Shigella NLAE-zl-p731, Escherichia/Shigella NLAE-zl-p826, Escherichia/Shigella NLAE-zl-p877, Escherichia/Shigella NLAE-zl-p884, Escherichia/Shigella NLAE-zl-pl26, Escherichia/Shigella NLAE-zl-pl98, Escherichia/Shigella NMU-ST2, Escherichia/Shigella ocl 82011, Escherichia/Shigella of, Escherichia/Shigella proteobacterium, Escherichia/Shigella Ql, Escherichia/Shigella sakazakii, Escherichia/Shigella SF6, Escherichia/Shigella sm1719, Escherichia/Shigella SOD-7317, Escherichia/Shigella sonnei, Escherichia/Shigella SW86, Escherichia/Shigella vulneris, Ethanoligenens, Ethanoligenens harbinense, Eubacterium, Eubacterium ARC-2, Eubacterium callanderi, Eubactenum E-l, Eubacterium G3(2011), Eubacterium infirmum, Eubacterium limosum, Eubacterium methylotrophicum, Eubacterium NLAE-zl-p439, Eubacterium NLAE-zl-p457, Eubacterium NLAE-zl-p458, Eubacterium NLAE-zl-p469 Eubacterium NLAE-zl-p474, Eubacterium oral, Eubacterium saphenum, Eubacterium sulci, Eubacterium WAL, Euglenida, Euglenida longa, Faecalibacterium, Faecalibacterium bacterium, Faecalibacterium canine, Faecalibacterium DJF VR20, Faecalibacterium icl379, Faecalibacterium prausnitzii, Filibacter, Filibacter globispora, Flavobacterium, Flavobacterium SSL03, Flavonifractor, Flavonifractor AUH-JLC235, Flavonifractor enrichment, Flavonifractor NLAE-zl-c354, Flavonifractor orbiscindens, Flavonifractor plautii, Francisella, Francisella piscicida, Fusobacterium, Fusobacterium nucleatum, Gardnerella, Gardnerella vaginalis, Gemmiger, Gemmiger DJF_VR33k2, Gemmiger formicilis, Geobacter, GHAPRB1, Gordonibacter, Gordonibacter bacterium, Gordonibacter intestinal, Gordonibacter pamelaeae, Gp2, Gp21, Gp4, Gp6, Granulicatella, Granulicatella adiacens, Granulicatella enrichment, Gramlicatella oral, Granulicatella paraadiacens, Haemophilus, Hafnia, Hafnia 3-12(2010), Hafnia alvei, Hafnia CCJ6, Hafnia proteus, Haliea, Hallella, Hallella seregens, Herbaspirillum, Herbaspirillum 022S4-11, Herbaspirillum seropedicae, Hespellia, Hespellia porcina, Hespellia stercorisuis, Holdemania, Holdemania AP2, Holdemania filiformis, Howardella, Howardella ureilvtica, Hydrogenoanaerobacterium, Hydrogenoanaerobacterium saccharovorans, Hvdrogenophaga, Hydrogenophaga bacterium, Ilumatobacter, Inulinivorans, Janthinobacterium, Janthinobacterium C30An7, Jeotgalicoccus, Klebsiella, Klebsiella aerogenes, Klebsiella bacterium, Klebsiella E1L1, Klebsiella FB2-THQ, Klebsiella enrichment, Klebsiella F83, Klebsiella ggl60e, Klebsiella Gl-6, Klebsiella granulomatis, Klebsiella HaNA20, Klebsiella HF2, Klebsiella ii_3_chl_l, Klebsiella KALAICIBAJ7, Klebsiella kpu, Klebsiella M3, Klebsiella MB45, Klebsiella milletis, Klebsiella NCCP-138, Klebsiella okl_l_9_S16, Klebsiella okl_l_9_S54, Klebsiella planticola, Klebsiella pneumoniae, Klebsiella poinarii, Klebsiella PSB26, Klebsiella RS, Klebsiella Sel4, Klebsiella SRC_DSD12, Klebsiella tdl53s, Klebsiella TG-1, Klebsiella TPS 5, Klebsiella variicola, Klebsiella WB-2, Klebsiella Y9, Klebsiella zlmy, Kluyvera, Kluyvera An5-1, Kluyvera cryocrescens, Kocuria, Kocuria 2216.35.31, Kurthia, Lachnobacterium, Lachnobacterium CJ2b, Lachnospiracea incertae sedis, Lachnospiracea incertae sedis bacterium, Lachnospiracea incertae sedis contortum, Lachnospiracea incertae sedis Eg2, Lachnospiracea incertae sedis eligens, Lachnospiracea incertae sedis ethanolgignens, Lachnospiracea incertae sedis galacturonicus, Lachnospiracea incertae sedis gnavus, Lachnospiracea incertae sedis hallii, Lachnospiracea incertae sedis hydrogenotrophica, Lachnospiracea incertae sedis ID5, Lachnospiracea incertae sedis intestinal, Lachnospiracea incertae sedis mpnisolate, Lachnospiracea incertae sedis pectinoschiza, Lachnospiracea incertae sedis ramulus, Lachnospiracea incertae sedis rectale, Lachnospiracea incertae sedis RLB1, Lachnospiracea incertae sedis rumen, Lachnospiracea ncertae sedis SY8519, Lachnospiracea incertae sedis torques, Lachnospiracea incertae sedis uniforme, Lachnospracea incertae sedis ventriosum, Lachnospiracea incertae sedis xylanophilum, Lachnospiracea incertae sedis ye62, Lactobacillus, Lactobacillus 5-1-2, Lactobacillus 66c, Lactobacillus acidophilus, Lactobacillus arizonensis, Lactobacillus B5406, Lactobacillus brevis, Lactobacillus casei, Lactobacillus crispatus, Lactobacillus curvatus, Lactobacillus delbrueckii, Lactobacillus fermentum, Lactobacillus gasseri, Lactobacillus helveticus, Lactobacillus hominis, Lactobacillus ID9203, Lactobacillus IDSAc, Lactobacillus intestinal, Lactobacillus johnsonii, Lactobacillus lactis, Lactobacillus manihotivorans, Lactobacillus mucosae, Lactobacillus NA, Lactobacillus oris, Lactobacillus P23, Lactobacillus P8, Lactobacillus paracasei, Lactobacillus paraplantarum, Lactobacillus pentosus, Lactobacillus plantarum, Lactobacillus pontis, Lactobacillus rennanqilfyl4, Lactobacillus rennanqilyf9, Lactobacillus reuteri, Lactobacillus rhamnosus, Lactobacillus salivarius, Lactobacillus sanranciscensis, Lactobacillus suntoryeus, lactobacillus T3R1C1, Lactobacillus vaginalis, Lactobacillus zeae, Lactococcus, Lactococcus 56, Lactococcus CR-317S, Lactococcus CW-1, Lactococcus D8, Lactococcus Da-18, Lactococcus DAP39, Lactococcus delbrueckii, Lactococcus F116, Lactococcus fujiensis, Lactococcus G22, Lactococcus garvieae, Lactococcus lactis, Lactococcus manure, Lactococcus RT5, Lactococcus SXVIII1(2011), Lactococcus TP2MJ, Lactococcus TP2MLA, Lactococcus TP2MN, Lactococcus U5-1, Lactonifactor, Lactonifactor bacterium, Lactonifactor longoviformis, Lactonifactor NLAE-zl-c533, Leclercia, Lentisphaera, Leuconostoc, Leuconostoc carnosum, Leuconostoc citreum, Leuconostoc garlicum, Leuconostoc gasicomitatum, Leuconostoc gelidum, Leuconostoc inhae, Leuconostoc lactis, Leuconostoc MEBE2, Leuconostoc mesenteroides, Leuconostoc pseudomesenteroides, Limnobacter, Limnobacter sp3, Luteolibacter, Luteolibacter bacterium, Lutispora, Marinfilum, Marinobacter, Marinobacter arcticus, Mariprofundus, Marvinbryantia, Megamonas, Megasphaera, Melissococcus, Melissococcus faecalis, Methanobacterium, Methanobacterium subterraneum, Methanobrevibacter, Afethanobrevibacter arboriphilus, Methanobrevibacter millerae, Methanobrevibacter olleyae, Methanobrevibacter oralis, Methanobrevibacter SM9, Methanobrevibacter smithii, Methanosphaera, Methanosphaera stadmanae, Methylobacterium, Methylobacterium adhaesivum, Methylobacterium bacterium, Methylobacterium iEID, Methylobacterium MP3, Methylobacterium oryzae, Methylobacterium PB32, Methylobacterium PB20, Methylobacterium PB280, Methylobacterium PDD-23b-14, Methylobacterium radiotolerans, Methylobacterium SKJH-1, Mitsuokella, Mitsuokella jalaludinii, Morganella, Morganella morganii, Moritella, Moritella 2D2, Moryella, Moryella indoligenes, Moryella naviforme, Mycobacterium, Mycobacterium tuberculosis, Negativicoccus, Nitrosomonas, Nitrosomonas eutropha, Novosphingobium, Odoribacter, Odoribacter laneus, Odoribacter splanchnicus, Olsenella, Olsenella 1832, Olsenella F0206, Orhus, Orbus gilliamella, Oribacterium, Oscillibacter, Oscillibacter bacterium, Oscillibacter enrichment, Owenweeksia, Oxalobacter, Oxalobacter formigenes, Paludibacter, Pantoea, Pantoea eucalypti, Papillibacter, Papillibacter cinnamivorans, Parabacteroides, Parabacteroides ASF519, Parabacteroides CR-34, Parabacteroides distasonis, Parabacteroides DJF B084, Parabacteroides DJF B086, Parabacteroides dnLKV8, Parabacteroides enrichment, Parabacteroides fecal, Parabacteroides goldsteinii, Parabacteroides gordonii, Parabacteroides johnsonii, Parabacteroides merdae, Parabacteroides mpnisolate, Parabacteroides NLAE-zl-p340, Paraeggerthella, Paraeggerthella hongkongensis, Paraeggerthella NLAE-zl-p797, Paraeggerthella NLAE-zl-p896, Paraprevotella, Paraprevotella clara, Parapreotella xylaniphila, Parasutterella, Parasutterella excrementihominis, Pectobacterium, Pectobacterium carotovorum, Pectobacterium wasabiae, Pediococcus, Pediococcus te2r, Pedobacter, Pedobacter b3Nlb-b5, Pedobacter daechungensis, Peptostreptococcus, Peptostreptococcus anaerobius, Peptostreptococcus stomatis, Phascolarctobacterium, Phascolarctobacterium faecium, Photobacterium, Photobacterium MIE, Pilibacter, Planctomyces, Planococcaceae incertae sedis, Planomicrobium, Plesiomonas, Porphyrobacter, Porphyrobacter KK348, Porphyromonas, Porphyromonas asaccharolylica, Porphyromonas bennonis, Porphyromonas canine, Porphyromonas somerae, Prevotella, Prevotella bacterium, Prevotella BI-42, Prevotella bivia, Prevotella buccalis, Prevotella copri, Prevotella DJF_B112, Prevotella mpnisolate, Prevotella oral, Propionibacterium, Propionibacterium acnes, Propionibacterium freudenreichii, Propionibacterium LG, Proteiniborus, Proteiniphilum, Proteus, Proteus HS7514, Providencia, Pseudobutyrivibrio, Pseudobutyrivibrio bacterium, Pseudobutyrivibrio fibrisolvens, Pseudobutyrivibrio ruminis, Pseudochrobactrum, Pseudoflavonifractor, Pseudoflavonifractor asf300, Pseudoflavonifractor bacterium, Pseudoflavonifractor capillosus, Pseudoflavonifractor NML, Pseudomonas, Pseudomonas 1043, Pseudomonas 10569, Pseudomonas 11-44, Pseudomonas 127(39-zx), Pseudomonas 12A_19, Pseudomonas 145(38zx), Pseudomonas 22010, Pseudomonas 32010, Pseudomonas 34t20, Pseudomonas 3C_10, Pseudomonas 4-5(2010), Pseudomonas 4-9(2010), Pseudomonas 6-13.J, Pseudomonas 63596, Pseudomonas 82010, Pseudomonas a001-142L, Pseudomonas aeruginosa, Pseudomonas agarici, Pseudomonas al l1-5, Pseudomonas alOl-18-2, Pseudomonas amspl, Pseudomonas A12390, Pseudomonas AZ8R1, Pseudomonas azotoformans, Pseudomonas B122, Pseudomonas B65(2012), Pseudomonas bacterium, Pseudomonas BJSX, Pseudomonas BLH-8D5, Pseudomonas BWDY-29 Pseudomonas CA18, Pseudomonas Cantasl2, Pseudomonas CB11, Pseudomonas CBZ-4, Pseudomonas cedrina, Pseudomonas CGMCC, Pseudomonas CL16, Pseudomonas CNE, Pseudomonas corrugata, Pseudomonas cualrocienegasensis, Pseudomonas CYEB-7, Pseudomonas D5, Pseudomonas DAP37, Pseudomonas DB48, Pseudomonas deceptionensis, Pseudomonas Den-05, Pseudomonas DF7EH1, Pseudomonas DhA-91, Pseudomonas DVS14a, Pseudomonas DYJK4-9, Pseudomonas DZQ5, Pseudomonas E11_ICE19B, Pseudomonas E2.2, Pseudomonas e2-CDC-TB4D2, Pseudomonas E1189, Pseudomonas enrichment, Pseudomonas extremorientalis, Pseudomonas FAIR/BE/F/GH37, Pseudomonas FAIR/BE/F/GH39, Pseudomonas FAIR/BE/F/GH94, Pseudomonas FLM05-3, Pseudomonas fluorescens, Pseudomonas fragi, Pseudomonas FSL, Pseudomonas G1013, Pseudomonas gingeri, Pseudomonas HC2-2, Pseudomonas HC2-4, Pseudomonas HC2-5, Pseudomonas HC4-8, Pseudomonas HC6-6, Pseudomonas Hg4-06, Pseudomonas HLB8-2, Pseudomonas HLS12-1, Pseudomonas HSF20-13, Pseudomonas HW08, Pseudomonas IpA-92, Pseudomonas IV, Pseudomonas JCM, Pseudomonas jessenii, Pseudomonas JSPB5, Pseudomonas K3R3.1A, Pseudomonas KB40, Pseudomonas KB42, Pseudomonas KB44, Pseudomonas KB63, Pseudomonas KB73, Pseudomonas KK-21-4, Pseudomonas KOPRI, Pseudomonas L1R3.5, Pseudomonas LAB-27, Pseudomonas LAB-44, Pseudomonas LclO-2, Pseudomonas libanensis, Pseudomonas Ln5C.7, Pseudomonas LS197, Pseudomonas lundensis, Pseudomonas marginalis, Pseudomonas MFY143, Pseudomonas MFY146, Pseudomonas MY 1412, Pseudomonas MY1404, Pseudomonas MY1416, Pseudomonas MY1420, Pseudomonas N14zhy, Pseudomonas NBRC, Pseudomonas NCCP-506, Pseudomonas NFU20-14, Pseudomonas NJ-22, Pseudomonas NJ-24, Pseudomonas Nj-3, Pseudomonas Nj-55, Pseudomonas Nj-56, Pseudomonas Nj-59, Pseudomonas Nj-60, Pseudomonas Nj-62, Pseudomonas Nj-70, Pseudomonas NP41, Pseudomonas OCW4, Pseudomonas OW3-15-3-2, Pseudomonas P2(2010), Pseudomonas P3(2010), Pseudomonas P4(2010), Pseudomonas PD, Pseudomonas PF1B4, Pseudomonas PF2M10, Pseudomonas PILH1, Pseudomonas Pl(2010), Pseudomonas poae, Pseudomonas proteobacterium, Pseudomonas ps4-12, Pseudomonas ps4-2, Pseudomonas ps4-28, Pseudomonas ps4-34, Pseudomonas ps4-4, Pseudomonas psychrophila, Pseudomonas putida, Pseudomonas R-35721, Pseudomonas R-37257, Pseudomonas R-37265, Pseudomonas R-37908, Pseudomonas RBE2CD-42, Pseudomonas regd9, Pseudomonas RKS7-3, Pseudomonas S2, Pseudomonas seawater, Pseudomonas SGb08, Pseudomonas SGb396, Pseudomonas SGbl20, Pseudomonas sgn, Pseudomonas Shk, Pseudomonas stutzer, Pseudomonas syringae, Pseudomonas taetrolens, Pseudomonas tolaasii, Pseudomonas triviahs, Pseudomonas TUT1023, Pseudomonas W15Feb26, Pseudomonas W15Feb4, Pseudomonas W15Feb6, Pseudomonas WD-3, Pseudomonas WR4-13, Pseudomonas WR7#2, Pseudomonas Y1000, Pseudomonas ZS29-8, Psychrobacter, Psychrobacter umbl3d, Pyramidobacter, Pyramidobacter piscolens, Rahnella, Rahnella aquatilis, Rahnella carotovorum, Rahnella GIST-WP4wl, Rahnella LR113, Rahnella Z2-S1, Ralstonia, Ralstonia bacterium, Raoultella, Raoultella B 19, Raoultella enrichment, Raoultella planticola, Raoultella sv6xvii, Raoultella SZ015, RBElCD-48, Renibacterium, Renibacterium G20, rennanqilfylO, Rhizobium, Rhizobium leguminosarum, Rhodococcus Rhodococcus erythropolis, Rhodopirellula, Riemerella, Riemerella anatipestifer, Rikenella, Robinsoniella, Robinsoniella peoriensis, Roseburia, Roseburia 11SE37, Roseburia bacterium, Roseburia cecicola, Roseburia DJF_VR77, Roseburia faecis, Roseburia fibrisolvens, Roseburia hominis, Roseburia intestinalis, Roseibacillus, Rothia, Rubritalea, Ruminococcus, Ruminococcus 25F6, Ruminococcus albus, Ruminococcus bacterium, Ruminococcus bromii, Ruminococcus callidus, Ruminococcus champanellensis, Ruminococcus DJF_VR87, Ruminococcus flavefaciens, Ruminococcus gauvreaui, Ruminococcus lactaris, Ruminococcus NK3A76, Ruminococcus YE71, Saccharoformentans, Salinicoccus, Salinimicrobium, Salmonella, Salmonella agglomerans, Salmonella bacterium, Salmonella enterica, Salmonella freundi, Salmonella hermannii, Salmonella paratyphi, Salmonella SL0604, Salmonella subterranea, Scardovia, Scardovia oral, Schwartzta, Sedimenticola, Sediminibacter, Selenomonas, Selenomonas fecal, Serpens, Serratia, Serratia 1135, Serratia 136-2, Serratia 5.1R, Serratia AC-CS-1B, Serratia AC-CS-B2, Serratia aquatilis, Serratia bacterium, Serratia BS26, Serratia carotovorum, Serratia DAP6, Serratia enrichment, Serratia F2, Serratia ficaria, Serratia fonticola, Serratia grimesii, Serratia J 145, Serratia JM983, Serratia liquefaciens, Serratia marcescens, Serratia plymuthica, Serratia proteamaculans, Serratia proteolvticus, Serratia ptz-16s, Serratia quinivorans, Serratia SBS, Serratia SS22, Serratia trout, Serratia UA-G004, Serratia White, Serratia yellow, Shewanella, Shewanella baltica, Slackia, Slackia intestinal, Slackia isolavoniconvertens, Slackia NATTS, Solibacillus, Solobacterium, Solobacterium moorei, Spartobacteria genera incertae sedis, Sphingobium, Sphingomonas, Sporacetigenium, Sporobacter, Sporobacterium, Sporobacterium olearium, Staphylococcus, Staphylococcus epidermidis, Staphylococcus PCA17, Stellenboschense, Stenotrophomonas, Streptococcus, Streptococcus 15, Streptococcus 1606-02B, Streptococcus agalactiae, Streptococcus alactolyticus, Streptococcus anginosus, Streptococcus bacterium, Streptococcus bovis, Streptococcus ChDC, Streptococcus constellatus, Streptococcus CR-314S, Streptococcus criceti, Streptococcus cristatus, Streptococcus downei, Strepococcus dysgalachae, Streptococcus enrichment, Streptococcus equi, Streptococcus equinus, Streptococcus ES11, Streptococcus eubacterium, Streptococcus fecal, Streptococcus gallinaceus, Streptococcus gallolvticus, Streptococcus gastrococcus, Streptococcus genomosp, Streptococcus gordonii, Streptococcus infantarius, Streptococcus intermedius, Streptococcus Je2, Streptococcus JS-CD2, Streptococcus LRC, Streptococcus luteciae, Streptococcus lutetiensis, Streptococcus M09-11185, Streptococcus mitis, Streptococcus mutans, Streptococcus NA, Streptococcus NLAE-zl-c353, Streptococcus NLAE-zl-p68, Streptococcus NLAE-zl-p758, Streptococcus NLAE-zl-p807, Streptococcus oral, Streptococcus oralis, Streptococcus parasanguinis, Streptococcus phocae, Streptococcus pneumoniae, Streptococcus porcinus, Streptococcus pyogenes, Streptococcus S 16-08, Streptococcus salivarius, Streptococcus sanguinis, Streptococcus sobrinus, Streptococcus suis, Streptococcus symbiont, Streptococcus thermophilus, Streptococcus TW1, Streptococcus vestibularis, Streptococcus warneri, Streptococcus XJ-RY-3, Streptomyces, Streptomyces malaysiensis, Streptomyces MVCS6, Streptophyta, Streptophyta cordifolium, Streptophyta ginseng, Streptophyta hirsutum, Streptophyta oleracea, Streptophyta satva, Streptophyta sativum, Streptophyta sativus, Streptophyta tabacum, Subdivision3 genera incertae sedis, Subdoligranulum, Subdoligranulum bacterium, Subdoligranulum icl393, Subdoligranulum ic1395, Subdoligranulum varabile, Succiniclasticum, Sulfuricella, Sulfuro spirillum, Suterella, Syntrophococcus, Svntrophomonas, Syntrophomonas bryantti, Syntrophus, Tannerella, Tatumella, Thermo gymnomonas, Thermofium, Thermogymnomonas, Thermovirga, Thiomonas, Thiomonas ML1-46, Thorsellia, Thorsellia carsonella, T7 genera incertae sedis, Trichococcus, Turicibacter, Turicibacter sanguinis, Vagococcus, Vagococcus bfsl l-15, Vampiro vibrio, Vampirovibrio, Varibaculum, Variovorax, Variovorax KS2D-23, Veillonella, Veillonella dispar, Veillonella MSA 12, Veillonella OK8, Veillonella oral, Veillonella parvula, Veillonella tobetsuensis, Vibrio, Vibrio 3C1, Victivallis, Victivallis vadensis, Vitellibacter, Wadsworthensis, Wandonia, Wandonia haliotis, Weissella, Weissella cibaria, Weissella confisa, Weissella oryzae, Yersinia, Yersinia 9gw38, Yersinia A125, Yersinia aldovae, Yersinia aleksiciae, Yersinia b702011, Yersinia bacterium, Yersinia bercovieri, Yersinia enterocolitica, Yersinia frederiksenii, Yersinia intermedia, Yersinia kristensenii, Yersinia MAC, Yersinia massiliensis, Yersinia mollaretti, Yersinia nurmii, Yersinia pekkaneni, Yersinia pestis, Yersinia pseudotuberculosis, Yersinia rohdei, Yersinia ruckeri, Yersinia s4fe31, Yersinia sl0fe31, Yersinia sl7fe31, and Yersinia YEM17B.

### Nucleic Acids

In some embodiments, therapeutic agents include nucleic acids. As used herein, the term “nucleic acid” refers to any polymer of nucleotides (natural or non-natural) or derivatives or variants thereof. Nucleic acids may include deoxyribonucleic acid (DNA) or ribonucleic acid (RNA). In some embodiments, nucleic acids may be polynucleotides or oligonucleotides. Some nucleic acids may include aptamers, plasmids, small interfering RNA (siRNA), microRNAs, or viral nucleic acids. In some embodiments, nucleic acids may encode proteins. In some embodiments, SBPs including therapeutic agent nucleic acids may include any of those described in International Publication Number WO2017123383, the contents of which are herein incorporated by reference in their entirety. In some embodiments, nucleic acids may include, but are not limited to, any of those listed in Table 3, above.

In some embodiments, nucleic acids may include a “CELiD” DNA as described in Li el al. (2013) PLoS One. 8(8):e69879, the contents of which are herein incorporated by reference in their entirety. CELiD DNA is a eukaryotic vector DNA that includes an expression cassette flanked by adeno-associated virus (AAV) inverted terminal repeats.

### Proteins

In some embodiments, SBPs may include biological agents that are or include proteins. As used herein, the term “protein” generally refers to polymers of amino acids linked by peptide bonds and embraces “peptides” and “polypeptides.” In some SBPs, the biological agent protein included is processed silk. Classes of proteins used as biological agent may include, but are not limited to, antigens, antibodies, antibody fragments, cytokines, peptides, hormones, enzymes, oxidants, antioxidants, synthetic proteins, and chimeric proteins. In some embodiments, proteins include any of those presented in Table 3, above. In some embodiments, proteins are combined with processed silk to improve protein stability.

In some embodiments, therapeutic agents are peptides. The term “peptide” generally refers to shorter proteins of about 50 amino acids or less. Peptides with only two amino acids may be referred to as “dipeptides.” Peptides with only three amino acids may be referred to as “tripeptides.” Polypeptides generally refer to proteins with from about 4 to about 50 amino acids. SBPs that include peptides may include any of those described in International Publication Numbers WO2017123383 and WO2010123945, the contents of each of which are herein incorporated by reference in their entirety. Peptides may be obtained via any method known to those skilled in the art. In some embodiments, peptides may be expressed in culture. In some embodiments, peptides may be obtained via chemical synthesis (e.g. solid phase peptide synthesis). In some embodiments, peptides are used to functionalize SBPs, for example, as taught in International Publication Number WO2010123945.

In some embodiments, SBPs are used to facilitate peptide delivery, for example, according to the methods presented in International Publication Number WO2017123383. In some embodiments, peptides include RGD peptides, for example, as taught in Kambe et al. (2017) Materials 10(10):1153, the contents of which are herein incorporated by reference in their entirety. Non-limiting examples of peptide therapeutic agents include, but are not limited to Degarelix acetate, Liraglutide, Cyclosporine, Eptifibatide, Dactinomycin, Spaglumat magnesium, Colistin, Nafarelin acetate, Somatostatin acetate, Buserclin, Enfuvirtide, Octreotide, Ianreotide acetate, Caspofungin, Nesiritide, Goserelin, Salmon calcitonin, Lepirudin or r-hirudin, Daptomycin, Exenatide, Carbetocin acetate, Tirofiban, Glutathione, Cetrorelix acetate, Enalapril maleate, Bivalirudin, Vapreotide acetate, Icatibant acetate, Human calcitonin, Oxytocin, Atosiban acetate, Bacitracin, Lypressin, Vancomycin, Captopril, Anidulafungin, Bortezomib, Saralasin acetate, Calcitonin, Thymalfasin, Ziconotide, and Lisinopril. In some embodiments, peptides may include any of those presented in Table 3, above.

In some embodiments, SBPs are used to deliver proteins. Non-limiting examples of proteins that may be delivered with SBPs include monoclonal antibodies, immunoglobulins (e.g., IgG), anti-VEGF antibodies (e.g., AVASTIN®), lysozyme, and bovine serum albumin (BSA). SBPs may provide controlled release of a stable protein over a desired administration period, for example, for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 1 month, at least 6 weeks, at least 2 months, at least 10 weeks, at least 3 months, at least 6 months, at least 9 months, or at least 1 year. In one embodiment, SBPs provide controlled release of a stable protein for at least 7 days.

SBP formulations used for peptide or protein delivery may be tailored based on variables such as the molecular weight of the peptide or protein to be delivered, the loading of the peptide or protein, the molecular weight of the silk fibroin, and the silk fibroin concentration used in the formulations.

### Synthetic/Chimeric Proteins

In some embodiments, therapeutic agents include synthetic proteins. As used herein, the term “synthetic” refers to any article produced through at least some human manipulation. Synthetic proteins may be identical to proteins found in nature or may have one or more distinguishing features. Distinguishing features may include, but are not limited to, differences in amino acid sequences, incorporation of non-natural amino acids, post-translational modifications, and conjugation to non-protein moieties (e.g., some antibody drug conjugates). Synthetic proteins may be expressed in vitro or in vivo. Synthetic proteins may also be chemically synthesized (e.g. by solid phase peptide synthesis). In some embodiments, synthetic proteins are made from a combination of expression and chemical synthesis (e.g. native chemical ligation or enzyme catalyzed protein ligation).

In some embodiments, synthetic proteins include chimeric or fusion proteins. As used herein, the term “fusion protein” refers to a substance that includes two or more protein components that are conjugated through at least one chemical bond. As used herein, the term “chimeric protein” refers to a protein that includes segments from at least two different sources (e.g., from two different species or two different isotypes or variants from a common species). Chimeric proteins may be produced via the expression of two or more ligated genes encoding different proteins. Chimeric proteins may be produced via chemical synthesis. In some embodiments, chimeric proteins are made from a combination of expression and chemical synthesis (e.g. native chemical ligation or enzyme catalyzed protein ligation). In some embodiments, synthetic proteins or chimeric proteins may include, but are not limited to, any of those listed in Table 3, above.

### Viruses and Viral Particles

In some embodiments, therapeutic agents are viruses or viral particles. Viruses and viral particles may be used to transfer nucleic acid into cells for genetic manipulation, gene therapy, gene editing, protein expression, or to inhibit protein expression. In some embodiments. SBPs be prepared with viral or viral particle payloads. In some embodiments, payload release may occur over a period of time (the payload release period). The payload release rate and/or length of the payload release period may be modulated by SBP components or methods of preparation. Examples of viruses and viral particles may include, but are not limited to, any of those presented in Table 3, above.

In some embodiments, the virus or viral particle payloads prepared with SBPs may include, but are not limited to, adeno-associated virus, lentivirus, alphavirus, enterovirus, pestivirus, baculovirus, herpesvirus, Epstein Barr virus, papovavirus, poxvirus, vaccinia virus, herpes simplex virus, and/or a viral particle thereof.

In some embodiments, the virus or viral particle may include an adeno-associated virus (AAV). A recombinant AAV vector can be used for the delivery of nucleic acids into cells. Methods for producing recombinant AAV particles are well-known in the art. Production of recombinant AAV particles typically requires the following components to be present within a single cell (also known as a packaging cell): a recombinant AAV genome, AAV rep (replication) and cap (capsid) genes separate from (i.e., not in) the recombinant AAV genome, and helper virus functions. The AAV rep and cap genes may be from any AAV serotype from which recombinant virus can be produced, and may be from a different AAV serotype than the recombinant AAV genome ITRs (i.e., inverted terminal repeats). Production of pseudotyped recombinant AAV is disclosed in, for example, WIPO Publication Number WO2001083692, the contents of which are hereby incorporated by reference in their entirety.

AAV particles packaging polynucleotides encoding a therapeutic agent (e.g., a peptide, a protein, or an antibody) of the invention may comprise or be derived from any natural or recombinant AAV serotype. The AAV particles may utilize or be based on a serotype selected from any of the following serotypes, and variants thereof, including, but not limited to, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53. AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1. AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2. AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57. AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7. AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42. AAVhu.43, AAVhu.44, AAVhu.44R1. AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63. AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1. AAVpi.2, AAVpi.3, AAVrh.10, AAVrh.12, AAVrh.13. AAVrh.13R. AAVrh.14, AAVrh.17, AAVrh.18, AAVrh.19, AAVrh.2, AAVrh.20, AAVrh.21. AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, avian AAV (AAAV), BNP61 AAV, BNP62 AAV, BNP63 AAV, bovine AAV (BAAV), caprine AAV, Japanese AAV 10, true type AAV (ttAAV), and/or UPENN AAV 10.

In some embodiments, the AAV serotype may be, or have, a sequence as described in U.S. Pat. No. 6,156,303, the contents of which are hereby incorporated by reference in their entirety, such as, but not limited to, AAV2 (SEQ ID NOs: 3 and 8 of U.S. Pat. No. 6,156,303), AAV3A (SEQ ID NOs: 4 and 9, of U.S. Pat. No. 6,156,303), AAV3B (SEQ ID NOs: 1 and 10 of U.S. Pat. No. 6,156,303), AAV6 (SEQ ID NOs: 2, 7 and 11 of U.S. Pat. No. 6,156,303), or derivatives thereof.

In some embodiments, the AAV serotype may be, or have, a variant of the AAV9 sequence as described by Pulicherla et al. (Molecular Therapy (2011) 19(6):1070-1078, the contents of which are hereby incorporated by reference in their entirety), such as, but not limited to, AAV9.9, AAV9.11, AAV9.13, AAV9.16, AAV9.24, AAV9.45, AAV9.47, AAV9.61, AAV9.68, and AAV9.84.

In one embodiment, the AAV may be a serotype generated by the AAV9 capsid library with one or more mutations in amino acids 390-627 (VP1 numbering) as described by Pulicherla et al. (Molecular Therapy (2011) 19(6):1070-1078, the contents of which are herein incorporated by reference in their entirety). The serotype and corresponding nucleotide and amino acid substitutions may be, but is not limited to, AAV9.1 (G1594C; D532H), AAV6.2 (T1418A, T1436X; V473D,1479K), AAV9.3 (T1238A; F413Y), AAV9.4 (T1250C, A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), AAV9.47 (G1241A. G1358A, A1669G, C1745T; S414N, G453D, K557E, T582I), AAV9.48 (C1445T, A1736T; P482L, Q579L), AAV9.50 (A1638T, C1683T, T1805A; Q546H L602H), AAV9.53 (G1301A, A1405C, C1664T, G1811T; R134Q, S469R, A555V, G604V), AAV9.54 (C153A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R T582S, D611V), AAV9.94 (A1675T; M559L), and AAV9.95 (T1605A; F535L), wherein nucleotide and amino acid substitutions are separated by “;” and “X” represents any nucleotide.

In some embodiments, the AAV serotype may be AAV-DJ or a variant thereof, such as AAV-DJ8 (or AAVDJ8), as described by Grimm et al. (Journal of Virology (2008) 82(12): 5887-5911, the contents of which are hereby incorporated by reference in their entirety). The amino acid sequence of AAV-DJ8 may comprise two or more mutations in the heparin binding domain (HBD) which result in the loss of heparin binding capability. As a non-limiting example, the AAV-DJ sequence described as SEQ ID NO: 1 in U.S. Pat. No. 7,588,772, the contents of which are herein incorporated by reference in their entirety, may comprise two amino acid mutations: R587Q and R590T. As another non-limiting example, the AAV-DJ sequence may comprise three amino acid mutations: K406R, R587Q and R590T.

In some embodiments, AAV capsid serotype may be isolated from a variety of species. In one embodiment, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NOs: 1, 2, 4, 6, 8, 10, 12, and 14 of U.S. Pat. No. 9,238,800), or variants thereof.

In one embodiment, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In one embodiment, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In some embodiments, the AAV serotype may be, or have, a sequence as described in WIPO Publication Number WO2015121501, the contents of which are hereby incorporated by reference in their entirety, such as, but not limited to, true type AAV (ttAAV) (SEQ ID NO: 2 of WO2015121501). “UPenn AAV10” (SEQ ID NO: 8 of WO2015121501), “Japanese AAV10” (SEQ ID NO: 9 of WO2015121501), or variants thereof.

In some embodiment, the AAV serotype may comprise at least one AAV capsid-specific CD8+ T-cell epitope. As non-limiting example, the serotype may be AAV1, AAV2 or AAV8.

In further embodiments, the AAV may be engineered as a hybrid AAV from two or more parental serotypes. In one embodiment, the AAV may be AAV2G9 which comprises sequences from AAV2 and AAV9. The AAV2G9 AAV serotype may be, or have, a sequence as described in US Patent Publication Number US2160017005, the contents of which are hereby incorporated by reference in their entirety.

In one embodiment, the AAV may be a serotype selected from any of those found in Table 4. In one embodiment, the AAV may be encoded by sequence, fragment or variant as described in Table 4.

TABLE 4
AAV Serotypes

| Serotype | Reference information for Serotype Sequence |
|---|---|
| AAAV (Avian AAV) | U.S. Pat. No. 9,238,800 SEQ ID NO: 12 |
| AAAV (Avian AAV) | U.S. Pat. No. 9,238,800 SEQ ID NO: 2 |
| AAAV (Avian AAV) | U.S. Pat. No. 9,238,800 SEQ ID NO: 6 |
| AAAV (Avian AAV) | U.S. Pat. No. 9,238,800 SEQ ID NO: 4 |
| AAAV (Avian AAV) | U.S. Pat. No. 9,238,800 SEQ ID NO: 8 |
| AAAV (Avian AAV) | U.S. Pat. No. 9,238,800 SEQ ID NO: 14 |
| AAAV (Avian AAV) | U.S. Pat. No. 9,238,800 SEQ ID NO: 10 |
| AAAV (Avian AAV) | U.S. Pat. No. 9,238,800 SEQ ID NO: 15 |
| AAAV (Avian AAV) | U.S. Pat. No. 9,238,800 SEQ ID NO: 5 |
| AAAV (Avian AAV) | U.S. Pat. No. 9,238,800 SEQ ID NO: 9 |
| AAAV (Avian AAV) | U.S. Pat. No. 9,238,800 SEQ ID NO: 3 |
| AAAV (Avian AAV) | U.S. Pat. No. 9,238,800 SEQ ID NO: 7 |
| AAAV (Avian AAV) | U.S. Pat. No. 9,238,800 SEQ ID NO: 11 |
| AAAV (Avian AAV) | U.S. Pat. No. 9,238,800 SEQ ID NO: 13 |
| AAAV (Avian AAV) | U.S. Pat. No. 9,238,800 SEQ ID NO: 1 |
| AAV Shuffle 100-1 | US20160017295 SEQ ID NO: 23 |
| AAV Shuffle 100-1 | US20160017295 SEQ ID NO: 11 |
| AAV Shuffle 100-2 | US20160017295 SEQ ID NO: 37 |
| AAV Shuffle 100-2 | US20160017295 SEQ ID NO: 29 |
| AAV Shuffle 100-3 | US20160017295 SEQ ID NO: 24 |
| AAV Shuffle 100-3 | US20160017295 SEQ ID NO: 12 |
| AAV Shuffle 100-7 | US20160017295 SEQ ID NO: 25 |
| AAV Shuffle 100-7 | US20160017295 SEQ ID NO: 13 |
| AAV Shuffle 10-2 | US20160017295 SEQ ID NO: 34 |
| AAV Shuffle 10-2 | US20160017295 SEQ ID NO: 26 |
| AAV Shuffle 10-6 | US20160017295 SEQ ID NO: 35 |
| AAV Shuffle 10-6 | US20160017295 SEQ ID NO: 27 |
| AAV Shuffle 10-8 | US20160017295 SEQ ID NO: 36 |
| AAV Shuffle 10-8 | US20160017295 SEQ ID NO: 28 |
| AAV SM 100-10 | US20160017295 SEQ ID NO: 41 |
| AAV SM 100-10 | US20160017295 SEQ ID NO: 33 |
| AAV SM 100-3 | US20160017295 SEQ ID NO: 40 |
| AAV SM 100-3 | US20160017295 SEQ ID NO: 32 |
| AAV SM 10-1 | US20160017295 SEQ ID NO: 38 |
| AAV SM 10-1 | US20160017295 SEQ ID NO: 30 |
| AAV SM 10-2 | US20160017295 SEQ ID NO: 10 |
| AAV SM 10-2 | US20160017295 SEQ ID NO: 22 |
| AAV SM 10-8 | US20160017295 SEQ ID NO: 39 |
| AAV SM 10-8 | US20160017295 SEQ ID NO: 31 |
| AAV1 | US20150159173 SEQ ID NO: 11, |
| | US20150315612 SEQ ID NO: 202 |
| AAV1 | US20160017295 SEQ ID NO: |
| | 1US20030138772 SEQ ID NO: 64, |
| | US20150159173 SEQ ID NO: 27, |
| | US20150315612 SEQ ID NO: 219, |
| | U.S. Pat. No. 7,198,951 SEQ ID NO: 5 |
| AAV1 | US20030138772 SEQ ID NO: 6 |
| AAV1.3 | US20030138772 SEQ ID NO: 14 |
| AAV10 | US20030138772 SEQ ID NO: 117 |
| AAV10 | WO2015121501 SEQ ID NO: 9 |
| AAV10 | WO2015121501 SEQ ID NO: 8 |
| AAV11 | US20030138772 SEQ ID NO: 118 |
| AAV12 | US20030138772 SEQ ID NO: 119 |
| AAV16.3 | US20030138772 SEQ ID NO: 105 |
| AAV2 | US20150159173 SEQ ID NO: 7, |
| | US20150315612 SEQ ID NO: 211 |
| AAV2 | US20030138772 SEQ ID NO: 70, |
| | US20150159173 SEQ ID NO: 23, |
| | US20150315612 SEQ ID NO: 221, |
| | US20160017295 SEQ ID NO: 2, |
| | U.S. Pat. No. 6,156,303 SEQ ID NO: 4, |
| | U.S. Pat. No. 7,198,951 SEQ ID NO: 4, |
| | WO2015121501 SEQ ID NO: 1 |
| AAV2 | U.S. Pat. No. 6,156,303 SEQ ID NO: 8 |
| AAV2 | US20030138772 SEQ ID NO: 7 |
| AAV2 | U.S. Pat. No. 6,156,303 SEQ ID NO: 3 |
| AAV2.5T | U.S. Pat. No. 9,233,131 SEQ ID NO: 42 |
| AAV223.10 | US20030138772 SEQ ID NO: 75 |
| AAV223.2 | US20030138772 SEQ ID NO: 49 |
| AAV223.2 | US20030138772 SEQ ID NO: 76 |
| AAV223.4 | US20030138772 SEQ ID NO: 50 |
| AAV223.4 | US20030138772 SEQ ID NO: 73 |
| AAV223.5 | US20030138772 SEQ ID NO: 51 |
| AAV223.5 | US20030138772 SEQ ID NO: 74 |
| AAV223.6 | US20030138772 SEQ ID NO: 52 |
| AAV223.6 | US20030138772 SEQ ID NO: 78 |
| AAV223.7 | US20030138772 SEQ ID NO: 53 |
| AAV223.7 | US20030138772 SEQ ID NO: 77 |
| AAV24.1 | US20030138772 SEQ ID NO: 101 |
| AAV27.3 | US20030138772 SEQ ID NO: 104 |
| AAV29.3 | US20030138772 SEQ ID NO: 82 |
| AAV29.3 (AAVbb.1) | US20030138772 SEQ ID NO: 11 |
| AAV29.4 | US20030138772 SEQ ID NO: 12 |
| AAV29.5 | US20030138772 SEQ ID NO: 83 |
| AAV29.5 (AAVbb.2) | US20030138772 SEQ ID NO: 13 |
| AAV3 | US20150159173 SEQ ID NO: 12 |
| AAV3 | US20030138772 SEQ ID NO: 71, |
| | US20150159173 SEQ ID NO: 28, |
| | US20160017295 SEQ ID NO: 3, |
| | U.S. Pat. No. 7,198,951 SEQ ID NO: 6 |
| AAV3 | US20030138772 SEQ ID NO: 8 |
| AAV3.3b | US20030138772 SEQ ID NO: 72 |
| AAV3-3 | US20150315612 SEQ ID NO: 200 |
| AAV3-3 | US20150315612 SEQ ID NO: 217 |
| AAV3a | U.S. Pat. No. 6,156,303 SEQ ID NO: 5 |
| AAV3a | U.S. Pat. No. 6,156,303 SEQ ID NO: 9 |
| AAV3b | U.S. Pat. No. 6,156,303 SEQ ID NO: 6 |
| AAV3b | U.S. Pat. No. 6,156,303 SEQ ID NO: 10 |
| AAV3b | U.S. Pat. No. 6,156,303 SEQ ID NO: 1 |
| AAV4 | US20140348794 SEQ ID NO: 17 |
| AAV4 | US20140348794 SEQ ID NO: 5 |
| AAV4 | US20140348794 SEQ ID NO: 3 |
| AAV4 | US20140348794 SEQ ID NO: 14 |
| AAV4 | US20140348794 SEQ ID NO: 15 |
| AAV4 | US20140348794 SEQ ID NO: 19 |
| AAV4 | US20140348794 SEQ ID NO: 12 |
| AAV4 | US20140348794 SEQ ID NO: 13 |
| AAV4 | US20140348794 SEQ ID NO: 7 |
| AAV4 | US20140348794 SEQ ID NO: 8 |
| AAV4 | US20140348794 SEQ ID NO: 9 |
| AAV4 | US20140348794 SEQ ID NO: 2 |
| AAV4 | US20140348794 SEQ ID NO: 10 |
| AAV4 | US20140348794 SEQ ID NO: 11 |
| AAV4 | US20140348794 SEQ ID NO: 18 |
| AAV4 | US20030138772 SEQ ID NO: 63, |
| | US20160017295 SEQ ID NO: 4, |
| | US20140348794 SEQ ID NO: 4 |
| AAV4 | US20140348794 SEQ ID NO: 16 |
| AAV4 | US20140348794 SEQ ID NO: 20 |
| AAV4 | US20140348794 SEQ ID NO: 6 |
| AAV4 | US20140348794 SEQ ID NO: 1 |
| AAV42.10 | US20030138772 SEQ ID NO: 106 |
| AAV42.11 | US20030138772 SEQ ID NO: 108 |
| AAV42.12 | US20030138772 SEQ ID NO: 113 |
| AAV42.13 | US20030138772 SEQ ID NO: 86 |
| AAV42.15 | US20030138772 SEQ ID NO: 84 |
| AAV42.1B | US20030138772 SEQ ID NO: 90 |
| AAV42.2 | US20030138772 SEQ ID NO: 9 |
| AAV42.2 | US20030138772 SEQ ID NO: 102 |
| AAV42.3A | US20030138772 SEQ ID NO: 87 |
| AAV42.3b | US20030138772 SEQ ID NO: 36 |
| AAV42.3B | US20030138772 SEQ ID NO: 107 |
| AAV42.4 | US20030138772 SEQ ID NO: 33 |
| AAV42.4 | US20030138772 SEQ ID NO: 88 |
| AAV42.5A | US20030138772 SEQ ID NO: 89 |
| AAV42.5B | US20030138772 SEQ ID NO: 91 |
| AAV42.6B | US20030138772 SEQ ID NO: 112 |
| AAV42.8 | US20030138772 SEQ ID NO: 27 |
| AAV42.8 | US20030138772 SEQ ID NO: 85 |
| AAV43.1 | US20030138772 SEQ ID NO: 39 |
| AAV43.1 | US20030138772 SEQ ID NO: 92 |
| AAV43.12 | US20030138772 SEQ ID NO: 41 |
| AAV43.12 | US20030138772 SEQ ID NO: 93 |
| AAV43.20 | US20030138772 SEQ ID NO: 42 |
| AAV43.20 | US20030138772 SEQ ID NO: 99 |
| AAV43.21 | US20030138772 SEQ ID NO: 43 |
| AAV43.21 | US20030138772 SEQ ID NO: 96 |
| AAV43.23 | US20030138772 SEQ ID NO: 44 |
| AAV43.23 | US20030138772 SEQ ID NO: 98 |
| AAV43.25 | US20030138772 SEQ ID NO: 45 |
| AAV43.25 | US20030138772 SEQ ID NO: 97 |
| AAV43.5 | US20030138772 SEQ ID NO: 40 |
| AAV43.5 | US20030138772 SEQ ID NO: 94 |
| AAV4-4 | US20150315612 SEQ ID NO: 201 |
| AAV4-4 | US20150315612 SEQ ID NO: 218 |
| AAV44.1 | US20030138772 SEQ ID NO: 46 |
| AAV44.1 | US20030138772 SEQ ID NO: 79 |
| AAV44.2 | US20030138772 SEQ ID NO: 59 |
| AAV44.5 | US20030138772 SEQ ID NO: 47 |
| AAV44.5 | US20030138772 SEQ ID NO: 80 |
| AAV4407 | US20150315612 SEQ ID NO: 90 |
| AAV5 | US20030138772 SEQ ID NO: 114 |
| AAV5 | US20160017295 SEQ ID NO: 5, |
| | U.S. Pat. No. 7,427,396 SEQ ID NO: 2, |
| | US20150315612 SEQ ID NO: 216 |
| AAV5 | U.S. Pat. No. 7,427,396 SEQ ID NO: 1 |
| AAV5 | US20150315612 SEQ ID NO: 199 |
| AAV6 | US20150159173 SEQ ID NO: 13 |
| AAV6 | US20030138772 SEQ ID NO: 65, |
| | US20150159173 SEQ ID NO: 29, |
| | US20160017295 SEQ ID NO: 6, |
| | U.S. Pat. No. 6,156,303 SEQ ID NO: 7 |
| AAV6 | U.S. Pat. No. 6,156,303 SEQ ID NO: 11 |
| AAV6 | U.S. Pat. No. 6,156,303 SEQ ID NO: 2 |
| AAV6 | US20150315612 SEQ ID NO: 220 |
| AAV6 | US20150315612 SEQ ID NO: 203 |
| AAV6.1 | US20150159173 |
| AAV6.12 | US20150159173 |
| AAV6.2 | US20150159173 |
| AAV7 | US20150159173 SEQ ID NO: 14 |
| AAV7 | US20150315612 SEQ ID NO: 183 |
| AAV7 | US20030138772 SEQ ID NO: 2, |
| | US20150159173 SEQ ID NO: 30, |
| | US20150315612 SEQ ID NO: 181, |
| | US20160017295 SEQ ID NO: 7 |
| AAV7 | US20030138772 SEQ ID NO: 3 |
| AAV7 | US20030138772 SEQ ID NO: 1, |
| | US20150315612 SEQ ID NO: 180 |
| AAV7 | US20150315612 SEQ ID NO: 213 |
| AAV7 | US20150315612 SEQ ID NO: 222 |
| AAV7.2 | US20030138772 SEQ ID NO: 103 |
| AAV8 | US20150159173 SEQ ID NO: 15 |
| AAV8 | US20150376240 SEQ ID NO: 7 |
| AAV8 | US20030138772 SEQ ID NO: 4, |
| | US20150315612 SEQ ID NO: 182 |
| AAV8 | US20030138772 SEQ ID NO: 95, |
| | US20140359799 SEQ ID NO: 1, |
| | US20150159173 SEQ ID NO: 31, |
| | US20160017295 SEQ ID NO: 8, |
| | U.S. Pat. No. 7,198,951 SEQ ID NO: 7, |
| | US20150315612 SEQ ID NO: 223 |
| AAV8 | US20150376240 SEQ ID NO: 8 |
| AAV8 | US20150315612 SEQ ID NO: 214 |
| AAV-8b | US20150376240 SEQ ID NO: 5 |
| AAV-8b | US20150376240 SEQ ID NO: 3 |
| AAV-8h | US20150376240 SEQ ID NO: 6 |
| AAV-8h | US20150376240 SEQ ID NO: 4 |
| AAV9 | US20030138772 SEQ ID NO: 5 |
| AAV9 | U.S. Pat. No. 7,198,951 SEQ ID NO: 1 |
| AAV9 | US20160017295 SEQ ID NO: 9 |
| AAV9 | US20030138772 SEQ ID NO: 100, |
| | U.S. Pat. No. 7,198,951 SEQ ID NO: 2 |
| AAV9 | U.S. Pat. No. 7,198,951 SEQ ID NO: 3 |
| AAV9 (AAVhu.14) | US20150315612 SEQ ID NO: 3 |
| AAV9 (AAVhu.14) | US20150315612 SEQ ID NO: 123 |
| AAVA3.1 | US20030138772 SEQ ID NO: 120 |
| AAVA3.3 | US20030138772 SEQ ID NO: 57 |
| AAVA3.3 | US20030138772 SEQ ID NO: 66 |
| AAVA3.4 | US20030138772 SEQ ID NO: 54 |
| AAVA3.4 | US20030138772 SEQ ID NO: 68 |
| AAVA3.5 | US20030138772 SEQ ID NO: 55 |
| AAVA3.5 | US20030138772 SEQ ID NO: 69 |
| AAVA3.7 | US20030138772 SEQ ID NO: 56 |
| AAVA3.7 | US20030138772 SEQ ID NO: 67 |
| AAVC1 | US20030138772 SEQ ID NO: 60 |
| AAVC2 | US20030138772 SEQ ID NO: 61 |
| AAVC5 | US20030138772 SEQ ID NO: 62 |
| AAVCh.5 | US20150159173 SEQ ID NO: 46, |
| | US20150315612 SEQ ID NO: 234 |
| AAVcy.2 (AAV13.3) | US20030138772 SEQ ID NO: 15 |
| AAVcy.3 (AAV24.1) | US20030138772 SEQ ID NO: 16 |
| AAVcy.4 (AAV27.3) | US20030138772 SEQ ID NO: 17 |
| AAVcy.5 | US20150315612 SEQ ID NO: 227 |
| AAVcy.5 | US20150159173 SEQ ID NO: 8 |
| AAVcy.5 | US20150159173 SEQ ID NO: 24 |
| AAVcy.5 (AAV7.2) | US20030138772 SEQ ID NO: 18 |
| AAVCy.5R1 | US20150159173 |
| AAVCy.5R2 | US20150159173 |
| AAVCy.5R3 | US20150159173 |
| AAVCy.5R4 | US20150159173 |
| AAVcy.6 (AAV16.3) | US20030138772 SEQ ID NO: 10 |
| AAVDJ | US20140359799 SEQ ID NO: 3, |
| | U.S. Pat. No. 7,588,772 SEQ ID NO: 2 |
| AAVDJ | US20140359799 SEQ ID NO: 2, |
| | U.S. Pat. No. 7,588,772 SEQ ID NO: 1 |
| AAVDJ-8 | U.S. Pat. No. 7,588,772; Grimm et al 2008 |
| AAVDJ-8 | U.S. Pat. No. 7,588,772; Grimm et al 2008 |
| AAVF1 | US20030138772 SEQ ID NO: 109 |
| AAVF3 | US20030138772 SEQ ID NO: 111 |
| AAVF5 | US20030138772 SEQ ID NO: 110 |
| AAVH2 | US20030138772 SEQ ID NO: 26 |
| AAVH6 | US20030138772 SEQ ID NO: 25 |
| AAVhE1.1 | U.S. Pat. No. 9,233,131 SEQ ID NO: 44 |
| AAVhEr1.14 | U.S. Pat. No. 9,233,131 SEQ ID NO: 46 |
| AAVhEr1.16 | U.S. Pat. No. 9,233,131 SEQ ID NO: 48 |
| AAVhEr4.18 | U.S. Pat. No. 9,233,131 SEQ ID NO: 49 |
| AAVhEr1.23 | U.S. Pat. No. 9,233,131 SEQ ID NO: 53 |
| (AAVhEr2.29) | |
| AAVhEr1.35 | U.S. Pat. No. 9,233,131 SEQ ID NO: 50 |
| AAVhEr1.36 | U.S. Pat. No. 9,233,131 SEQ ID NO: 52 |
| AAVhEr1.5 | U.S. Pat. No. 9,233,131 SEQ ID NO: 45 |
| AAVhEr1.7 | U.S. Pat. No. 9,233,131 SEQ ID NO: 51 |
| AAVhEr1.8 | U.S. Pat. No. 9,233,131 SEQ ID NO: 47 |
| AAVhEr2.16 | U.S. Pat. No. 9,233,131 SEQ ID NO: 55 |
| AAVhEr2.30 | U.S. Pat. No. 9,233,131 SEQ ID NO: 56 |
| AAVhEr2.31 | U.S. Pat. No. 9,233,131 SEQ ID NO: 58 |
| AAVhEr2.36 | U.S. Pat. No. 9,233,131 SEQ ID NO: 57 |
| AAVhEr2.4 | U.S. Pat. No. 9,233,131 SEQ ID NO: 54 |
| AAVhEr3.1 | U.S. Pat. No. 9,233,131 SEQ ID NO: 59 |
| AAVhu.1 | US20150315612 SEQ ID NO: 46 |
| AAVhu.1 | US20150315612 SEQ ID NO: 144 |
| AAVhu.10 | US20150315612 SEQ ID NO: 56 |
| (AAV16.8) | |
| AAVhu.10 | US20150315612 SEQ ID NO: 156 |
| (AAV16.8) | |
| AAVhu.11 | US20150315612 SEQ ID NO: 57 |
| (AAV16.12) | |
| AAVhu.11 | US20150315612 SEQ ID NO: 153 |
| (AAV16.12) | |
| AAVhu.12 | US20150315612 SEQ ID NO: 59 |
| AAVhu.12 | US20150315612 SEQ ID NO: 154 |
| AAVhu.13 | US20150159173 SEQ ID NO: 16, |
| | US20150315612 SEQ ID NO: 71 |
| AAVhu.13 | US20150159173 SEQ ID NO: 32, |
| | US20150315612 SEQ ID NO: 129 |
| AAVhu.136.1 | US20150315612 SEQ ID NO: 165 |
| AAVhu.140.1 | US20150315612 SEQ ID NO: 166 |
| AAVhu.140.2 | US20150315612 SEQ ID NO: 167 |
| AAVhu.145.6 | US20150315612 SEQ ID No: 178 |
| AAVhu.15 | US20150315612 SEQ ID NO: 147 |
| AAVhu.15 | US20150315612 SEQ ID NO: 50 |
| (AAV33.4) | |
| AAVhu.156.1 | US20150315612 SEQ ID No: 179 |
| AAVhu.16 | US20150315612 SEQ ID NO: 148 |
| AAVhu.16 | US20150315612 SEQ ID NO: 51 |
| (AAV33.8) | |
| AAVhu.17 | US20150315612 SEQ ID NO: 83 |
| AAVhu.17 | US20150315612 SEQ ID NO: 4 |
| (AAV33.12) | |
| AAVhu.172.1 | US20150315612 SEQ ID NO: 171 |
| AAVhu.172.2 | US20150315612 SEQ ID NO: 172 |
| AAVhu.173.4 | US20150315612 SEQ ID NO: 173 |
| AAVhu.173.8 | US20150315612 SEQ ID NO: 175 |
| AAVhu.18 | US20150315612 SEQ ID NO: 52 |
| AAVhu.18 | US20150315612 SEQ ID NO: 149 |
| AAVhu.19 | US20150315612 SEQ ID NO: 62 |
| AAVhu.19 | US20150315612 SEQ ID NO: 133 |
| AAVhu.2 | US20150315612 SEQ ID NO: 48 |
| AAVhu.2 | US20150315612 SEQ ID NO: 143 |
| AAVhu.20 | US20150315612 SEQ ID NO: 63 |
| AAVhu.20 | US20150315612 SEQ ID NO: 134 |
| AAVhu.21 | US20150315612 SEQ ID NO: 65 |
| AAVhu.21 | US20150315612 SEQ ID NO: 135 |
| AAVhu.22 | US20150315612 SEQ ID NO: 67 |
| AAVhu.22 | US20150315612 SEQ ID NO: 138 |
| AAVhu.23 | US20150315612 SEQ ID NO: 60 |
| AAVhu.23.2 | US20150315612 SEQ ID NO: 137 |
| AAVhu.24 | US20150315612 SEQ ID NO: 66 |
| AAVhu.24 | US20150315612 SEQ ID NO: 136 |
| AAVhu.25 | US20150315612 SEQ ID NO: 49 |
| AAVhu.25 | US20150315612 SEQ ID NO: 146 |
| AAVhu.26 | US20150159173 SEQ ID NO: 17, |
| | US20150315612 SEQ ID NO: 61 |
| AAVhu.26 | US20150159173 SEQ ID NO: 33, |
| | US20150315612 SEQ ID NO: 139 |
| AAVhu.27 | US20150315612 SEQ ID NO: 64 |
| AAVhu.27 | US20150315612 SEQ ID NO: 140 |
| AAVhu.28 | US20150315612 SEQ ID NO: 68 |
| AAVhu.28 | US20150315612 SEQ ID NO: 130 |
| AAVhu.29 | US20150315612 SEQ ID NO: 69 |
| AAVhu.29 | US20150159173 SEQ ID NO: 42, |
| | US20150315612 SEQ ID NO: 132 |
| AAVhu.29 | US20150315612 SEQ ID NO: 225 |
| AAVhu.29R | US20150159173 |
| AAVhu.3 | US20150315612 SEQ ID NO: 44 |
| AAVhu.3 | US20150315612 SEQ ID NO: 145 |
| AAVhu.30 | US20150315612 SEQ ID NO: 70 |
| AAVhu.30 | US20150315612 SEQ ID NO: 131 |
| AAVhu.31 | US20150315612 SEQ ID NO: 1 |
| AAVhu.31 | US20150315612 SEQ ID NO: 121 |
| AAVhu.32 | US20150315612 SEQ ID NO: 2 |
| AAVhu.32 | US20150315612 SEQ ID NO: 122 |
| AAVhu.33 | US20150315612 SEQ ID NO: 75 |
| AAVhu.33 | US20150315612 SEQ ID NO: 124 |
| AAVhu.34 | US20150315612 SEQ ID NO: 72 |
| AAVhu.34 | US20150315612 SEQ ID NO: 125 |
| AAVhu.35 | US20150315612 SEQ ID NO: 73 |
| AAVhu.35 | US20150315612 SEQ ID NO: 164 |
| AAVhu.36 | US20150315612 SEQ ID NO: 74 |
| AAVhu.36 | US20150315612 SEQ ID NO: 126 |
| AAVhu.37 | US20150159173 SEQ ID NO: 34, |
| | US20150315612 SEQ ID NO: 88 |
| AAVhu.37 | US20150315612 SEQ ID NO: 10, |
| (AAV106.1) | US20150159173 SEQ ID NO: 18 |
| AAVhu.38 | US20150315612 SEQ ID NO: 161 |
| AAVhu.39 | US20150315612 SEQ ID NO: 102 |
| AAVhu.39 | US20150315612 SEQ ID NO: 24 |
| (AAVLG-9) | |
| AAVhu.4 | US20150315612 SEQ ID NO: 47 |
| AAVhu.4 | US20150315612 SEQ ID NO: 141 |
| AAVhu.40 | US20150315612 SEQ ID NO: 87 |
| AAVhu.40 | US20150315612 SEQ ID No: 11 |
| (AAV114.3) | |
| AAVhu.41 | US20150315612 SEQ ID NO: 91 |
| AAVhu.41 | US20150315612 SEQ ID NO: 6 |
| (AAV127.2) | |
| AAVhu.42 | US20150315612 SEQ ID NO: 85 |
| AAVhu.42 | US20150315612 SEQ ID NO: 8 |
| (AAV127.5) | |
| AAVhu.43 | US20150315612 SEQ ID NO: 160 |
| AAVhu.43 | US20150315612 SEQ ID NO: 236 |
| AAVhu.43 | US20150315612 SEQ ID NO: 80 |
| (AAV128.1) | |
| AAVhu.44 | US20150159173 SEQ ID NO: 45, |
| | US20150315612 SEQ ID NO: 158 |
| AAVhu.44 | US20150315612 SEQ ID NO: 81 |
| (AAV128.3) | |
| AAVhu.44R1 | US20150159173 |
| AAVhu.44R2 | US20150159173 |
| AAVhu.44R3 | US20150159173 |
| AAVhu.45 | US20150315612 SEQ ID NO: 76 |
| AAVhu.45 | US20150315612 SEQ ID NO: 127 |
| AAVhu.46 | US20150315612 SEQ ID NO: 82 |
| AAVhu.46 | US20150315612 SEQ ID NO: 159 |
| AAVhu.46 | US20150315612 SEQ ID NO: 224 |
| AAVhu.47 | US20150315612 SEQ ID NO: 77 |
| AAVhu.47 | US20150315612 SEQ ID NO: 128 |
| AAVhu.48 | US20150159173 SEQ ID NO: 38 |
| AAVhu.48 | US20150315612 SEQ ID NO: 157 |
| AAVhu.48 | US20150315612 SEQ ID NO: 78 |
| (AAV130.4) | |
| AAVhu.48R1 | US20150159173 |
| AAVhu.48R2 | US20150159173 |
| AAVhu.48R3 | US20150159173 |
| AAVhu.49 | US20150315612 SEQ ID NO: 209 |
| AAVhu.49 | US20150315612 SEQ ID NO: 189 |
| AAVhu.5 | US20150315612 SEQ ID NO: 45 |
| AAVhu.5 | US20150315612 SEQ ID NO: 142 |
| AAVhu.51 | US20150315612 SEQ ID NO: 208 |
| AAVhu.51 | US20150315612 SEQ ID NO: 190 |
| AAVhu.52 | US20150315612 SEQ ID NO: 210 |
| AAVhu.52 | US20150315612 SEQ ID NO: 191 |
| AAVhu.53 | US20150159173 SEQ ID NO: 19 |
| AAVhu.53 | US20150159173 SEQ ID NO: 35 |
| AAVhu.53 | US20150315612 SEQ ID NO: 176 |
| (AAV145.1) | |
| AAVhu.54 | US20150315612 SEQ ID NO: 188 |
| AAVhu.54 | US20150315612 SEQ ID No: 177 |
| (AAV145.5) | |
| AAVhu.55 | US20150315612 SEQ ID NO: 187 |
| AAVhu.56 | US20150315612 SEQ ID NO: 205 |
| AAVhu.56 | US20150315612 SEQ ID NO: 168 |
| (AAV145.6) | |
| AAVhu.56 | US20150315612 SEQ ID NO: 192 |
| (AAV145.6) | |
| AAVhu.57 | US20150315612 SEQ ID NO: 206 |
| AAVhu.57 | US20150315612 SEQ ID NO: 169 |
| AAVhu.57 | US20150315612 SEQ ID NO: 193 |
| AAVhu.58 | US20150315612 SEQ ID NO: 207 |
| AAVhu.58 | US20150315612 SEQ ID NO: 194 |
| AAVhu.6 (AAV3.1) | US20150315612 SEQ ID NO: 5 |
| AAVhu.6 (AAV3.1) | US20150315612 SEQ ID NO: 84 |
| AAVhu.60 | US20150315612 SEQ ID NO: 184 |
| AAVhu.60 | US20150315612 SEQ ID NO: 170 |
| (AAV161.10) | |
| AAVhu.61 | US20150315612 SEQ ID NO: 185 |
| AAVhu.61 | US20150315612 SEQ ID NO: 174 |
| (AAV161.6) | |
| AAVhu.63 | US20150315612 SEQ ID NO: 204 |
| AAVhu.63 | US20150315612 SEQ ID NO: 195 |
| AAVhu.64 | US20150315612 SEQ ID NO: 212 |
| AAVhu.64 | US20150315612 SEQ ID NO: 196 |
| AAVhu.66 | US20150315612 SEQ ID NO: 197 |
| AAVhu.67 | US20150315612 SEQ ID NO: 215 |
| AAVhu.67 | US20150315612 SEQ ID NO: 198 |
| AAVhu.7 | US20150315612 SEQ ID NO: 226 |
| AAVhu.7 | US20150315612 SEQ ID NO: 150 |
| AAVhu.7 (AAV7.3) | US20150315612 SEQ ID NO: 55 |
| AAVhu.71 | US20150315612 SEQ ID NO: 79 |
| AAVhu.8 | US20150315612 SEQ ID NO: 53 |
| AAVhu.8 | US20150315612 SEQ ID NO: 12 |
| AAVhu.8 | US20150315612 SEQ ID NO: 151 |
| AAVhu.9 (AAV3.1) | US20150315612 SEQ ID NO: 58 |
| AAVhu.9 (AAV3.1) | US20150315612 SEQ ID NO: 155 |
| AAV-LK01 | US20150376607 SEQ ID NO: 2 |
| AAV-LK01 | US20150376607 SEQ ID NO: 29 |
| AAV-LK02 | US20150376607 SEQ ID NO: 3 |
| AAV-LK02 | US20150376607 SEQ ID NO: 30 |
| AAV-LK03 | US20150376607 SEQ ID NO: 4 |
| AAV-LK03 | WO2015121501 SEQ ID NO: 12, |
| | US20150376607 SEQ ID NO: 31 |
| AAV-LK04 | US20150376607 SEQ ID NO: 5 |
| AAV-LK04 | US20150376607 SEQ ID NO: 32 |
| AAV-LK05 | US20150376607 SEQ ID NO: 6 |
| AAV-LK05 | US20150376607 SEQ ID NO: 33 |
| AAV-LK06 | US20150376607 SEQ ID NO: 7 |
| AAV-LK06 | US20150376607 SEQ ID NO: 34 |
| AAV-LK07 | US20150376607 SEQ ID NO: 8 |
| AAV-LK07 | US20150376607 SEQ ID NO: 35 |
| AAV-LK08 | US20150376607 SEQ ID NO: 9 |
| AAV-LK08 | US20150376607 SEQ ID NO: 36 |
| AAV-LK09 | US20150376607 SEQ ID NO: 10 |
| AAV-LK09 | US20150376607 SEQ ID NO: 37 |
| AAV-LK10 | US20150376607 SEQ ID NO: 11 |
| AAV-LK10 | US20150376607 SEQ ID NO: 38 |
| AAV-LK11 | US20150376607 SEQ ID NO: 12 |
| AAV-LK11 | US20150376607 SEQ ID NO: 39 |
| AAV-LK12 | US20150376607 SEQ ID NO: 13 |
| AAV-LK12 | US20150376607 SEQ ID NO: 40 |
| AAV-LK13 | US20150376607 SEQ ID NO: 14 |
| AAV-LK13 | US20150376607 SEQ ID NO: 41 |
| AAV-LK14 | US20150376607 SEQ ID NO: 15 |
| AAV-LK14 | US20150376607 SEQ ID NO: 42 |
| AAV-LK15 | US20150376607 SEQ ID NO: 16 |
| AAV-LK15 | US20150376607 SEQ ID NO: 43 |
| AAV-LK16 | US20150376607 SEQ ID NO: 17 |
| AAV-LK16 | US20150376607 SEQ ID NO: 44 |
| AAV-LK17 | US20150376607 SEQ ID NO: 18 |
| AAV-LK17 | US20150376607 SEQ ID NO: 45 |
| AAV-LK18 | US20150376607 SEQ ID NO: 19 |
| AAV-LK18 | US20150376607 SEQ ID NO: 46 |
| AAV-LK19 | US20150376607 SEQ ID NO: 20 |
| AAV-LK19 | US20150376607 SEQ ID NO: 47 |
| AAV-PAEC | US20150376607 SEQ ID NO: 1 |
| AAV-PAEC | US20150376607 SEQ ID NO: 48 |
| AAV-PAEC11 | US20150376607 SEQ ID NO: 26 |
| AAV-PAEC11 | US20150376607 SEQ ID NO: 54 |
| AAV-PAEC12 | US20150376607 SEQ ID NO: 27 |
| AAV-PAEC12 | US20150376607 SEQ ID NO: 51 |
| AAV-PAEC13 | US20150376607 SEQ ID NO: 28 |
| AAV-PAEC13 | US20150376607 SEQ ID NO: 49 |
| AAV-PAEC2 | US20150376607 SEQ ID NO: 21 |
| AAV-PAEC2 | US20150376607 SEQ ID NO: 56 |
| AAV-PAEC4 | US20150376607 SEQ ID NO: 22 |
| AAV-PAEC4 | US20150376607 SEQ ID NO: 55 |
| AAV-PAEC6 | US20150376607 SEQ ID NO: 23 |
| AAV-PAEC6 | US20150376607 SEQ ID NO: 52 |
| AAV-PAEC7 | US20150376607 SEQ ID NO: 24 |
| AAV-PAEC7 | US20150376607 SEQ ID NO: 53 |
| AAV-PAEC8 | US20150376607 SEQ ID NO: 25 |
| AAV-PAEC8 | US20150376607 SEQ ID NO: 50 |
| AAVpi.1 | US20150315612 SEQ ID NO: 28 |
| AAVpi.1 | US20150315612 SEQ ID NO: 93 |
| AAVpi.2 | US20150315612 SEQ ID NO: 30 |
| AAVpi.2 | US20150315612 SEQ ID NO: 95 |
| AAVpi.3 | US20150315612 SEQ ID NO: 29 |
| AAVpi.3 | US20150315612 SEQ ID NO: 94 |
| AAVrh.10 | US20150159173 SEQ ID NO: 9 |
| AAVrh.10 | US20150159173 SEQ ID NO: 25 |
| AAVrh.10 (AAV44.2) | US20030138772 SEQ ID NO: 81 |
| AAVrh.12 | US20030138772 SEQ ID NO: 30 |
| (AAV42.1b) | |
| AAVrh.13 | US20150159173 SEQ ID NO: 10 |
| AAVrh.13 | US20150159173 SEQ ID NO: 26 |
| AAVrh.13 | US20150315612 SEQ ID NO: 228 |
| AAVrh.13R | US20150159173 |
| AAVrh.14 | US20030138772 SEQ ID NO: 32 |
| (AAV42.3a) | |
| AAVrh.17 | US20030138772 SEQ ID NO: 34 |
| (AAV42.5a) | |
| AAVrh.18 | US20030138772 SEQ ID NO: 29 |
| (AAV42.5b) | |
| AAVrh.19 | US20030138772 SEQ ID NO: 38 |
| (AAV42.6b) | |
| AAVrh.2 | US20150159173 SEQ ID NO: 39 |
| AAVrh.2 | US20150315612 SEQ ID NO: 231 |
| AAVrh.20 | US20150159173 SEQ ID NO: 1 |
| AAVrh.21 | US20030138772 SEQ ID NO: 35 |
| (AAV42.10) | |
| AAVrh.22 | US20030138772 SEQ ID NO: 37 |
| (AAV42.11) | |
| AAVrh.23 | US20030138772 SEQ ID NO: 58 |
| (AAV42.12) | |
| AAVrh.24 | US20030138772 SEQ ID NO: 31 |
| (AAV42.13) | |
| AAVrh.25 | US20030138772 SEQ ID NO: 28 |
| (AAV42.15) | |
| AAVrh.2R | US20150159173 |
| AAVrh.31 | US20030138772 SEQ ID NO: 48 |
| (AAV22.3.1) | |
| AAVrh.32 (AAVC1) | US20030138772 SEQ ID NO: 19 |
| AAVrh.32/33 | US20150159173 SEQ ID NO: 2 |
| AAVrh.33 (AAVC3) | US20030138772 SEQ ID NO: 20 |
| AAVrh.34 (AAVC5) | US20030138772 SEQ ID NO: 21 |
| AAVrh.35 (AAVF1) | US20030138772 SEQ ID NO: 22 |
| AAVrh.36 (AAVF3) | US20030138772 SEQ ID NO: 23 |
| AAVrh.37 | US20030138772 SEQ ID NO: 24 |
| AAVrh.37 | US20150159173 SEQ ID NO: 40 |
| AAVrh.37 | US20150315612 SEQ ID NO: 229 |
| AAVrh.37R2 | US20150159173 |
| AAVrh.38 | US20150315612 SEQ ID NO: 7 |
| (AAVLG-4) | |
| AAVrh.38 | US20150315612 SEQ ID NO: 86 |
| (AAVLG-4) | |
| AAVrh.39 | US20150159173 SEQ ID NO: 20, |
| | US20150315612 SEQ ID NO: 13 |
| AAVrh.39 | US20150159173 SEQ ID NO: 3, |
| | US20150159173 SEQ ID NO: 36, |
| | US20150315612 SEQ ID NO: 89 |
| AAVrh.40 | US20150315612 SEQ ID NO: 92 |
| AAVrh.40 | US20150315612 SEQ ID No: 14 |
| (AAVLG-10) | |
| AAVrh.43 | US20150315612 SEQ ID NO: 43, |
| (AAVN721-8) | US20150159173 SEQ ID NO: 21 |
| AAVrh.43 | US20150315612 SEQ ID NO: 163, |
| (AAVN721-8) | US20150159173 SEQ ID NO: 37 |
| AAVrh.44 | US20150315612 SEQ ID NO: 34 |
| AAVrh.44 | US20150315612 SEQ ID NO: 111 |
| AAVrh.45 | US20150315612 SEQ ID NO: 41 |
| AAVrh.45 | US20150315612 SEQ ID NO: 109 |
| AAVrh.46 | US20150159173 SEQ ID NO: 22, |
| | US20150315612 SEQ ID NO: 19 |
| AAVrh.46 | US20150159173 SEQ ID NO: 4, |
| | US20150315612 SEQ ID NO: 101 |
| AAVrh.47 | US20150315612 SEQ ID NO: 38 |
| AAVrh.47 | US20150315612 SEQ ID NO: 118 |
| AAVrh.48 | US20150159173 SEQ ID NO: 44, |
| | US20150315612 SEQ ID NO: 115 |
| AAVrh.48 (AAV1-7) | US20150315612 SEQ ID NO: 32 |
| AAVrh.48.1 | US20150159173 |
| AAVrh.48.1.2 | US20150159173 |
| AAVrh.48.2 | US20150159173 |
| AAVrh.49 (AAV1-8) | US20150315612 SEQ ID NO: 25 |
| AAVrh.49 (AAV1-8) | US20150315612 SEQ ID NO: 103 |
| AAVrh.50 (AAV2-4) | US20150315612 SEQ ID NO: 23 |
| AAVrh.50 (AAV2-4) | US20150315612 SEQ ID NO: 108 |
| AAVrh.51 (AAV2-5) | US20150315612 SEQ ID No: 22 |
| AAVrh.51 (AAV2-5) | US20150315612 SEQ ID NO: 104 |
| AAVrh.52 (AAV3-9) | US20150315612 SEQ ID NO: 18 |
| AAVrh.52 (AAV3-9) | US20150315612 SEQ ID NO: 96 |
| AAVrh.53 | US20150315612 SEQ ID NO: 97 |
| AAVrh.53 (AAV3-11) | US20150315612 SEQ ID NO: 17 |
| AAVrh.53 (AAV3-11) | US20150315612 SEQ ID NO: 186 |
| AAVrh.54 | US20150315612 SEQ ID NO: 40 |
| AAVrh.54 | US20150159173 SEQ ID NO: 49, |
| | US20150315612 SEQ ID NO: 116 |
| AAVrh.55 | US20150315612 SEQ ID NO: 37 |
| AAVrh.55 (AAV4-19) | US20150315612 SEQ ID NO: 117 |
| AAVrh.56 | US20150315612 SEQ ID NO: 54 |
| AAVrh.56 | US20150315612 SEQ ID NO: 152 |
| AAVrh.57 | US20150315612 SEQ ID NO: 26 |
| AAVrh.57 | US20150315612 SEQ ID NO: 105 |
| AAVrh.58 | US20150315612 SEQ ID NO: 27 |
| AAVrh.58 | US20150159173 SEQ ID NO: 48, |
| | US20150315612 SEQ ID NO: 106 |
| AAVrh.58 | US20150315612 SEQ ID NO: 232 |
| AAVrh.59 | US20150315612 SEQ ID NO: 42 |
| AAVrh.59 | US20150315612 SEQ ID NO: 110 |
| AAVrh.60 | US20150315612 SEQ ID NO: 31 |
| AAVrh.60 | US20150315612 SEQ ID NO: 120 |
| AAVrh.61 | US20150315612 SEQ ID NO: 107 |
| AAVrh.61 (AAV2-3) | US20150315612 SEQ ID NO: 21 |
| AAVrh.62 (AAV2-15) | US20150315612 SEQ ID No: 33 |
| AAVrh.62 (AAV2-15) | US20150315612 SEQ ID NO: 114 |
| AAVrh.64 | US20150315612 SEQ ID No: 15 |
| AAVrh.64 | US20150159173 SEQ ID NO: 43, |
| | US20150315612 SEQ ID NO: 99 |
| AAVrh.64 | US20150315612 SEQ ID NO: 233 |
| AAVRh.64R1 | US20150159173 |
| AAVRh.64R2 | US20150159173 |
| AAVrh.65 | US20150315612 SEQ ID NO: 35 |
| AAVrh.65 | US20150315612 SEQ ID NO: 112 |
| AAVrh.67 | US20150315612 SEQ ID NO: 36 |
| AAVrh.67 | US20150315612 SEQ ID NO: 230 |
| AAVrh.67 | US20150159173 SEQ ID NO: 47, |
| | US20150315612 SEQ ID NO: 113 |
| AAVrh.68 | US20150315612 SEQ ID NO: 16 |
| AAVrh.68 | US20150315612 SEQ ID NO: 100 |
| AAVrh.69 | US20150315612 SEQ ID NO: 39 |
| AAVrh.69 | US20150315612 SEQ ID NO: 119 |
| AAVrh.70 | US20150315612 SEQ ID NO: 20 |
| AAVrh.70 | US20150315612 SEQ ID NO: 98 |
| AAVrh.71 | US20150315612 SEQ ID NO: 162 |
| AAVrh.72 | US20150315612 SEQ ID NO: 9 |
| AAVrh.73 | US20150159173 SEQ ID NO: 5 |
| AAVrh.74 | US20150159173 SEQ ID NO: 6 |
| AAVrh.8 | US20150159173 SEQ ID NO: 41 |
| AAVrh.8 | US20150315612 SEQ ID NO: 235 |
| AAVrh.8R | US20150159173, |
| | WO2015168666 SEQ ID NO: 9 |
| AAVrh.8R A586R | WO2015168666 SEQ ID NO: 10 |
| mutant | |
| AAVrh.8R R533A | WO2015168666 SEQ ID NO: 11 |
| mutant | |
| BAAV (bovine AAV) | U.S. Pat. No. 9,193,769 SEQ ID NO: 11 |
| BAAV (bovine AAV) | U.S. Pat. No. 7,427,396 SEQ ID NO: 5 |
| BAAV (bovine AAV) | U.S. Pat. No. 7,427,396 SEQ ID NO: 6 |
| BAAV (bovine AAV) | U.S. Pat. No. 9,193,769 SEQ ID NO: 8 |
| BAAV (bovine AAV) | U.S. Pat. No. 9,193,769 SEQ ID NO: 10 |
| BAAV (bovine AAV) | U.S. Pat. No. 9,193,769 SEQ ID NO: 4 |
| BAAV (bovine AAV) | U.S. Pat. No. 9,193,769 SEQ ID NO: 2 |
| BAAV (bovine AAV) | U.S. Pat. No. 9,193,769 SEQ ID NO: 6 |
| BAAV (bovine AAV) | U.S. Pat. No. 9,193,769 SEQ ID NO: 1 |
| BAAV (bovine AAV) | U.S. Pat. No. 9,193,769 SEQ ID NO: 5 |
| BAAV (bovine AAV) | U.S. Pat. No. 9,193,769 SEQ ID NO: 3 |
| BAAV (bovine AAV) | U.S. Pat. No. 9,193,769 SEQ ID NO: 7 |
| BAAV (bovine AAV) | U.S. Pat. No. 9,193,769 SEQ ID NO: 9 |
| BNP61 AAV | US20150238550 SEQ ID NO: 1 |
| BNP61 AAV | US20150238550 SEQ ID NO: 2 |
| BNP62 AAV | US20150238550 SEQ ID NO: 3 |
| BNP63 AAV | US20150238550 SEQ ID NO: 4 |
| caprine AAV | U.S. Pat. No. 7,427,396 SEQ ID NO: 3 |
| caprine AAV | U.S. Pat. No. 7,427,396 SEQ ID NO: 4 |
| true type AAV | WO2015121501 SEQ ID NO: 2 |
| (ttAAV) | |

Each of the patents, applications and/or publications listed in Table 4 are hereby incorporated by reference in their entirety.

AAV vector serotypes may be formulated with SBPs for the delivery into specific tissue and/or cell types. As non-limiting examples, liver cells may be transduced by AAV3, AAV8, and/or AAV9; skeletal muscle cells may be transduced by AAV1, AAV7, AAV6, AAV8, and/or AAV9; cells of the central nervous system may be transduced by AAV5, AAV1, and/or AAV4; retinal pigment epithelium cells may be transduced by AAV5 and/or AAV4; photoreceptor cells may be transduced by AAV5; lung cells may be transduced by AAV9; heart cells may be transduced by AAV8; pancreatic cells may be transduced by AAV8; and kidney cells may be transduced by AAV2. Any of these AAV serotypes may be prepared SBPs of the present invention to facilitate delivery of such particles to the target tissue and/or cell types.

In some embodiments, the virus or viral particle may include a lentivirus. The lentivirus may comprise or be derived from human immunodeficiency virus (HIV), simian immunodeficiency virus (SIV), simian AIDS retrovirus SRV-1, feline immunodeficiency virus (FIV), Caprine arthritis encephalitis virus (CAEV), Bovine immunodeficiency virus (BIV), and Visna/maedi virus, and the like.

### Oxidants/Antioxidants

In some embodiments, therapeutic agents include oxidants or antioxidants. As used herein, the term “oxidant” refers to a substance that oxidizes (i.e., strips electrons from) another substance. Inhibitors of oxidation are referred to herein as “antioxidants.” The use of oxidants and/or antioxidants as therapeutic agents may include any of the methods taught, for example, in International Publication Number WO2017137937; Min et al. (2017) Int J Biol Macromol s0141-8130(17):32855-32856; or Manchineella et at (2017) European Journal of Organic Chemistry 30:4363-4369, the contents of each of which are herein incorporated by reference in their entirety. Oxidant or antioxidant therapeutic agents may be included in SBPs for treatment of indications requiring localized treatment or for indications requiring activity more distant from an administration site. In some embodiments, incorporation of oxidants or antioxidants may be used to modulate SBPs stability or degradation. In some embodiments, oxidants or antioxidants may be polymers. Such polymers may include quaternary ammonium chitosan and melanin. Examples of such therapeutic agents include those taught in International Publication Number WO2017137937 and Min et al. (2017) Int J Biol Macromol s0141-8130(17):32855-32856, the contents of each of which are herein incorporated by reference in their entirety. In some embodiments, oxidants or antioxidants include small molecules, metals, ions, minerals, vitamins, peptides, and/or proteins. In some embodiments, antioxidants include cyclic dipeptides or 2,5-diketopiperazines. Such antioxidants may include any of those taught in Manchineella et al. (2017) European Journal of Organic Chemistry 30:4363-4369, the contents of which are herein incorporated by reference in their entirety. In some embodiments, oxidants or antioxidants may include, but are not limited to, any of those listed in Table 3, above.

### Small Molecules

In some embodiments, SBPs include small molecule therapeutic agents. As used herein, the term “small molecule” refers to a low molecular weight compound, typically less than 900 Daltons. Many small molecules are able to pass through cell membranes, making them attractive candidates for therapeutic applications. SBPs may be combined with any small molecules to carry out a variety of therapeutic applications. Such small molecules may include small molecule drugs approved for human treatment. Some small molecules may be hydrophobic or hydrophilic. Small molecules may include, but are not limited to, antibacterial agents, antifungal agents, anti-inflammatory agents, nonsteroidal anti-inflammatory drugs, antipyretics, analgesics, antimalarial agents, antiseptics, hormones, stimulants, tranquilizers, and statins. In some embodiments, small molecules may include any of those listed in Table 3, above.

In some embodiments, SBPs may be used to encapsulate, store and/or release, in a controlled manner, small molecules. For example, using silk fibroin micrococoons as delivery vehicles for small molecules has been described in Shimanovich et al. (Shimanovich et al. (2015) Nature Communications 8:15902, the contents of which are herein incorporated by reference in their entirety).

### Angiogenesis Modulators

In some embodiments, therapeutic agents include modulators of angiogenesis. Such therapeutic agents may include vascular endothelial growth factor (VEGF)-related agents. As used herein, the term “VEGF-related agent” refers to any substance that affects VEGF expression, synthesis, stability, biological activity, degradation, receptor binding, cellular signaling, transport, secretion, internalization, concentration, or deposition (e.g., in extracellular matrix).

In some embodiments, VEGF-related agents are angiogenesis inhibitors. In some embodiments, the angiogenesis inhibitor includes any of those taught in International Publication Number WO2013126799, the contents of which are herein incorporated by reference in their entirety. In some embodiments, VEGF-related agents may include antibodies. VEGF-related agents may include VEGF agonists, including, but not limited to, toll-like receptor agonists. In some embodiments, the therapeutic agent is a VEGF antagonist. VEGF agonists or antagonists may be small molecules. In some embodiments, VEGF agonists or antagonists may be macromolecules or proteins. Angiogenesis inhibitors may include, but are not limited to, MACUGEN® or another VEGF nucleic acid ligand; LUCENTIS®, AVASTIN®, or another anti-VEGF antibody; combretastatin or a derivative or prodrug thereof such as Combretastatin A4 Prodrug (CA4P); VEGF-Trap (Regeneron); EVIZON™ (squalamine lactate); AG-013958 (Pfizer, Inc.); JSM6427 (Jerini AG); a short interfering RNA (siRNA) that inhibits expression of one or more VEGF isoforms (e.g., VEGF₁₆₅); an siRNA that inhibits expression of a VEGF receptor (e.g., VEGFR1), endogenous or synthetic peptides, angiostatin, combstatin, arresten, tumstatin, thalidomide, thalidomide derivatives, canstatin, endostatin, thrombospondin, and β2-glycoprotein 1. In some embodiments, VEGF-related agents may include, but are not limited to any of those listed in Table 3, above.

### Antibacterial Agents

In some embodiments, therapeutic agents include antibacterial agents. As used herein, the term “antibacterial agent” refers to any substance that harms, kills, or otherwise inhibits the growth and/or reproduction of bacteria. Anti-bacterial agents may include, but are not limited to, any of those listed in Table 3, above.

### Antifungal Agents

In some embodiments, therapeutic agents include antifungal agents. As used herein, the term “antifungal agent” refers to any substance that harms, kills, or otherwise inhibits the growth and/or reproduction of fungi. Antifungal agents may include, but are not limited to, any of those listed in Table 3, above.

### Analgesic Agents

In some embodiments, therapeutic agents include analgesic agents. As used herein, the term “analgesic agent” refers to any substance used to reduce or alleviate pain. Analgesic agents may include, but are not limited to, any of those listed in Table 3, above.

### Antipyretics

In some embodiments, therapeutic agents include antipyretics. As used herein, the term “antipyretic” refers to any substance used to reduce or alleviate fever. Examples of antipyretics include, but are not limited to, any NSAID, acetaminophen, aspirin and related salicylates (e.g. choline salicylate, magnesium salicylate, and sodium salicylate), ibuprofen, naproxen, ketoprofen, nimesulide, phenazone, metamizole, and nabumetone. In some embodiments, antipyretics may include, but are not limited to, any of those listed in Table 3, above.

### Antimalarial Agents

In some embodiments, therapeutic agents include antimalarial agents. As used herein, the term “antimalarial agent” refers to any substance that harms, kills, or otherwise inhibits the growth and/or reproduction of Plasmodium parasites. Examples of antimalarial agents may include, but are not limited to, any of those listed in Table 3, above.

### Antiseptic Agents

In some embodiments, therapeutic agents include antiseptic agents. As used herein, the term “antiseptic agent” refers to any substance that harms, kills, or otherwise inhibits the growth and/or reproduction of microorganisms. Examples of antiseptics include, but are not limited to, iodine, lower alcohols (ethanol, propanol, etc.), chlorhexidine, quaternary amine surfactants, chlorinated phenols, biguanides, bisbiguanides polymeric quaternary ammonium compounds, silver and its complexes, small molecule quaternary ammonium compounds, peroxides, and hydrogen peroxide. In some embodiments, antiseptic agents may include any of those listed in Table 3, above.

### Hormones

In some embodiments, therapeutic agents include hormones. As used herein, the term “hormone” refers to a cellular signaling molecule that promotes a response in cells or tissues. Hormones may be produced naturally by cells. In some embodiments, hormones are synthetic. Examples of hormones include, but are not limited to, any steroid, dexamethasone, allopregnanolone, any estrogen (e.g. ethinyl estradiol, mestranol, estradiols and their esters, estriol, estriol succinate, polyestriol phosphate, estrone, estrone sulfate and conjugated estrogens), any progestogen (e.g. progesterone, norethisterone acetate, norgestrel, levonorgestrel, gestodene, chlormadinone acetate, drospirorenone, and 3-ketodesogestrel), any androgen (e.g. testosterone, androstenediol, androstenedione, dehydroepiandrosterone, and dihydrotestosterone), any mineralocorticoid, any glucocoriticoid, cholesterols, and any hormone known to those skilled in the art. In some embodiments, hormones may include, but are not limited to, any of those listed in Table 3, above.

### Non-Steroidal Anti-Inflammatory Drugs

In some embodiments, therapeutic agents include nonsteroidal anti-inflammatory drugs. A nonsteroidal anti-inflammatory drug (NSAID) is a class of non-opioid analgesics used to reduce inflammation and associated pain. NSAIDs may include, but are not limited to, any of those listed in Table 3, above. In some embodiments, the NSAID is celecoxib. Some SBPs include gels or hydrogels that are combined with NSAIDs (e.g., celecoxib). Such SBPs may be used as carriers for NSAID payload delivery. NSAID delivery may include controlled release of the NSAID.

### Ocular Therapeutic Agents

In some embodiments, therapeutic agents include ocular therapeutic agents. As used herein, the term “ocular therapeutic agent” refers to any compound that has a healing, corrective, diagnostic, and/or prophylactic effect and/or elicits a desired biological and/or pharmacological effect on the eye. In some embodiments, ocular therapeutic agents include one or more of processed silk, biological agents, small molecules, proteins, nonsteroidal anti-inflammatory drugs, and vascular endothelial growth factor-related agents. Ocular therapeutic agent proteins may include, but are not limited to, lysozyme, bovine serum albumin (BSA), bevacizumab, or VEGF-related agents. In some embodiments, ocular therapeutic agents may be used to treat one or more of the ocular therapeutic indications described herein.

### Stimulants

In some embodiments, therapeutic agents include stimulants. As used herein, the term “stimulant” refers to any substance that increases subject physiological or nervous activity. Examples of stimulants include, but are not limited to, amphetamines, caffeine, ephedrine, 3,4-methylenedioxymethamphetamine, methylenedioxypyrovalerone, mephedrone, methamphetamine, methylphenidate, nicotine, phenylpropanolamine, propylhexedrine, pseudoephedrine, and cocaine. In some embodiments, stimulants may include, but are not limited to, any of those listed in Table 3, above.

### Tranquilizers

In some embodiments, therapeutic agents include tranquilizers. As used herein, the term “tranquilizer” refers to any substance used to lower subject anxiety or tension. Examples of tranquilizers include, but are not limited to, barbiturates, benzodiazepines, carbamates, antihistamines, opioids, antidepressants (e.g. monoamine oxidase inhibitors, tetracyclic antidepressants, tricyclic antidepressants, selective serotonin reuptake inhibitors, and serotonin-norepinephrine reuptake inhibitors), sympatholytics (e.g. alpha blockers, beta-blockers, and alpha-adrenergic agonists), mebicar, fabomotizole, selank, bromantane, emoxypine, azapirones, pregabalin, mentyl isovalerate, propofol, racetams, alcohols, inhalants, any butyrophenone (e.g. benperidol, bromperidol, droperidol, haloperidol, moperone, pipamperone, and timiperone), any diphenylbutylpiperidine (e.g. fluspirilene, penfluridol, and pimozide), any phenothiazine (e.g. acepromazine, chlorpromazine, cyamemazine, dixyrazine, fluphenazine, levomepromazine, levomepromazine, mesoridazine, perazine, periciazine, perphenazine, pipotiazine, prochlorperazine, promazine, promethazine, prothipendyl, thioproperazine, thioridazine, trifluoperazine, and triflupromazine), any thioxanthene (e.g. chlorprothixene, clopenthixol, flupentixol, thiothixene, and zuclopenthixol), any benzamidine (e.g. sulpiride, sultopride, and veralipride), any tricyclic (e.g. carpipramine, clocapramine, clorotepine, loxapine, and mosapramine), gamma aminobutyric acid, and molindone. In some embodiments, tranquilizers may include, but are not limited to, any of those listed in Table 3, above.

### Statins

In some embodiments, therapeutic agents include statins. As used herein, the term “statin” refers to a class of compounds that inhibit hydroxy-methylglutaryl-coenzyme A reductase (HMG-CoA reductase), a key enzyme in cholesterol biosynthesis. Statins are referred to herein in the broadest sense and include statin derivatives such as ester derivatives or protected ester derivatives. Examples of statins include, but are not limited to, rosuvastatin, pitavastatin, pravastatin, fluvastatin, cerivastatin, atorvastatin, simvastatin, mevastatin, and lovastatin. In some embodiments, statins may include, but are not limited to, any of those listed in Table 3, above.

### Anti-Cancer Agents

In some embodiments, therapeutic agents include anticancer agents. As used herein, the term “anticancer agent” refers to any substance used to kill cancer cells or inhibit cancer cell growth and/or cell division. Anticancer agents that target tumor cells are referred to herein as “antitumor agents.” Such anticancer agents may reduce tumor mass and/or volume. Anticancer agents that are chemical substances are referred to herein as “chemotherapeutic agents.” Examples of antitumor agents include, but are not limited to, busulphan, cisplatin, cyclophosphamide, MTX, daunorubicin, doxorubicin, melphalan, vincristine, vinblastine, chlorabucil, any alkylating agent (e.g. cyclophosphamide, mechlorethamine, chlorambucil, melphalan, dacarbazine, nitrosoureas, and temozolomide), any anthracycline (e.g. daunorubicin, doxorubicin, epirubicin, idarubicin, mitozantrone, and valrubicin), any cytoskeletal disruptors or taxanes (e.g. paclitaxel, docetaxel, abraxane, and taxotere), any epothilones, any histone deacetylase inhibitors (e.g. vorinostat and romidepsin), any topoisomerase I inhibitors (e.g. irinotecan and topotecan), any topoisomerase II inhibitors (e.g. etoposide, teniposide, and tafluposide), kinase inhibitors (e.g. bortezomib, erlotinib, gefitinib, imatinib, vemurafenib, and vismodegib), nucleotide and precursor analogues (e.g. azacitidine, azathioprine, capecitabine, cytarabine, doxifluridine, fluorouracil, gemcitabine, hydroxyurea, mercaptopurine, methotrexate, and tioguanine), antimicrobial peptides (e.g. bleomycin and actinomycin), platinum based chemotherapeutics (e.g. carboplatin, cisplatin, oxaliplatin), retinoids (e.g. tretinoin, alitretinoin, and bexarotene), and vinca alkaloids and derivatives (e.g. vinblastine, vincristine, vindesine, and vinorelbine). In some embodiments, anticancer agents may include, but are not limited to, any of those listed in Table 3, above.

### Herbal Preparations

In some embodiments, therapeutic agents include herbal preparations. As used herein, the term “herbal preparation” refers to any substance derived or extracted from vegetation. These preparations may include, but are not limited to, tea, decoctions, cold infusions, tinctures, cordials, herbal wines, granules, syrups, essential oils (e.g. allspice berry essential oil, angelica seed essential oil, anise seed essential oil, basil essential oil, bay laurel essential oil, bay essential oil, bergamot essential oil, blood orange essential oil, camphor essential oil, caraway seed essential oil, cardamom seed essential oil, carrot seed essential oil, cassia essential oil, catnip essential oil, cedarwood essential oil, celery seed essential oil, chamomile german essential oil, chamomile roman essential oil, cinnamon bark essential oil, cinnamon leaf essential oil, citronella essential oil, clary sage essential oil, clove bud essential oil, coriander seed essential oil, cypress essential oil, elemi essential oil, eucalyptus essential oil, fennel essential oil, fir needle essential oil, frankincense essential oil, geranium essential oil, ginger essential oil, grapefruit pink essential oil, helichrysum essential oil, hop essential oil, hyssop essential oil, juniper berry essential oil, labdanum essential oil, lemon essential oil, lemongrass essential oil, lime essential oil, magnolia essential oil, mandarin essential oil, margoram essential oil, Melissa essential oil, mugward essential oil, myrrh essential oil, myrtle essential oil, neroli essential oil, niaouli essential oil, nutmeg essential oil, orange sweet essential oil, oregano essential oil, palmarosa essential oil, patchouli essential oil, pennyroyal essential oil, pepper black essential oil, peppermint essential oil, petitgram essential oil, pine needle essential oil, radiata essential oil, ravensara essential oil, rose essential oil, rosemary essential oil, rosewood essential oil, sage essential oil, sandalwood essential oil, spearmint essential oil, spikenard essential oil, spruce essential oil, star anise essential oil, sweet annie essential oil, tangerine essential oil, tea tree essential oil, thyme red essential oil, verbena essential oil, vetiver essential oil, wintergreen essential oil, wormwood essential oil, yarrow essential oil, ylang essential oil, jasmine absolute oil, lavender absolute oil, pink lotus absolute oil, rose absolute oil, sambac absolute oil, and white lotus absolute oil), flower essences, sitz baths, soaks, pills, suppositories, poultices, compresses, salves, and ointments. Examples of herbs to be incorporated include, but are not limited to, sage, thyme, cumin, basil, bay laurel, borage, caraway, catnip, chervil, chives, cilantro, dill, epazote, fennel, garlic, lavender, lemongrass, lemon balm, lemon verbena, lovage, marjoram, mints, nasturtium, parsley, oregano, rosemary, salad burnet, savory, scented geranium, sorrel, and tarragon. In some embodiments, herbal preparations may include, but are not limited to, any of those listed in Table 3, above.

### Health Supplements

In some embodiments, therapeutic agents include health supplements. As used herein, the term “health supplement” refers to any substance used to provide a nutrient, vitamin, or other beneficial compound that is typically lacking from a normal diet or is complimentary to such substances present in a normal diet. Examples of health supplements include, but are not limited to, vitamin A, vitamin B, vitamin C, vitamin D, vitamin E, vitamin K, thiamin, riboflavin, niacin, vitamin B6, vitamin B12, biotin, pantothenic acid, calcium, iron, phosphorus, iodine, magnesium, zinc, selenium, selenium, copper, manganese, chromium, molybdenum, chloride, potassium, nickel, silicon, vanadium, and tin. In some embodiments, health supplements may include, but are not limited to, any of those listed in Table 3, above.

### Ions, Metals, Minerals

In some embodiments, therapeutic agents include ions, metals, and/or minerals. Examples include, but are not limited to, calcium, iron, phosphorus, iodine, magnesium, zinc, selenium, selenium, copper, manganese, chromium, molybdenum, gold, silver, chloride, potassium, nickel, silicon, vanadium, and tin. In some embodiments, therapeutic agents include oxides (e.g. silver oxide). In some embodiments, ions, metals, and/or minerals may be present in nanoparticles. Such nanoparticles may include any of those taught in Mane et al. (2017) Scientific Reports 7:15531; and Babu et al. (2017) J Colloid Interface Sci 513:62-72, the contents of each of which are herein incorporated by reference in their entirety. In some embodiments, ions, metals, and/or minerals may include, but are not limited to, any of those listed in Table 3, above.

### Vitamins

In some embodiments, therapeutic agents include vitamins or vitamin analogues. As used herein, the term “vitamin” refers to a nutrient that must be obtained through diet (i.e., is not synthesized endogenously or is synthesized endogenously, but in insufficient amounts). Examples of vitamins include, but are not limited to, vitamin A, vitamin B-1, vitamin B-2, vitamin B-3, vitamin B-5, vitamin B-6, vitamin B-7, vitamin B-9, vitamin B-12, vitamin C, vitamin D, vitamin E, and vitamin K. In some embodiments, vitamins may include, but are not limited to, any of those listed in Table 3, above.

### Therapeutic Indications

In some embodiments, SBPs are used to address one or more therapeutic indications. As used herein, the term “therapeutic indication” refers to a disease, disorder, condition, or symptom that may be cured, reversed, alleviated, stabilized, improved, or otherwise addressed through some form of therapeutic intervention (e.g., administration of a therapeutic agent or method of treatment).

SBP treatment of therapeutic indications may include contacting subjects with SBPs. SBPs may include therapeutic agents (e.g., any of those described herein) as cargo or payloads for treatment. In some embodiments, payload release may occur over a period of time (the “payload release period”). The payload release rate and/or length of the payload release period may be modulated by SBP components or methods of preparation.

In some embodiments, therapeutic indications may include, but are not limited to, any of those listed in Table 5. In the Table, example categories are indicated for each therapeutic indication. These categories are not limiting and each therapeutic indication may fall under multiple categories (e.g., any of the categories of therapeutic indication described herein).

TABLE 5
Therapeutic indications

| Therapeutic indication | Category |
|---|---|
| food allergy | allergy |
| skin allergy | allergy |
| dust allergy | allergy |
| insect allergy | allergy |
| pet allergy | allergy |
| eye allergy | allergy |
| skin allergy | allergy |
| drug allergy | allergy |
| latex allergy | allergy |
| allergic rhinitis | allergy |
| mold allergy | allergy |
| sinus infection | allergy |
| cockroach allergy | allergy |
| hay fever | allergy |
| pollen allergy | allergy |
| sinusitis | allergy |
| asthma | allergy |
| insect sting or venom allergy | allergy |
| skin contact allergy | allergy |
| eczema | allergy |
| dermatitis | allergy |
| allergic conjunctivitis | allergy |
| chemical sensitivity | allergy |
| autoimmune disease | autoimmune |
| Acute Disseminated Encephalomyelitis (ADEM) | autoimmune |
| Acute necrotizing hemorrhagic leukoencephalitis | autoimmune |
| Addison's disease | autoimmune |
| Agammaglobulinemia | autoimmune |
| Alopecia areata | autoimmune |
| Amyloidosis | autoimmune |
| Ankylosing spondylitis | autoimmune |
| Anti-GBM/Anti-TBM nephritis | autoimmune |
| Antiphospholipid syndrome (APS) | autoimmune |
| Autoimmune angioedema | autoimmune |
| Autoimmune aplastic anemia | autoimmune |
| Autoimmune dysautonomia | autoimmune |
| Autoimmune hepatitis | autoimmune |
| Autoimmune hyperlipidemia | autoimmune |
| Autoimmune immunodeficiency | autoimmune |
| Autoimmune inner ear disease (AIED) | autoimmune |
| Autoimmune myocarditis | autoimmune |
| Autoimmune oophoritis | autoimmune |
| Autoimmune pancreatitis | autoimmune |
| Autoimmune retinopathy | autoimmune |
| Autoimmune thrombocytopenic purpura (ATP) | autoimmune |
| Autoimmune thyroid disease | autoimmune |
| Autoimmune urticaria | autoimmune |
| Axonal neuropathy | autoimmune |
| neuronal neuropathy | autoimmune |
| axonal and neuronal neuropathy | autoimmune |
| Balo disease | autoimmune |
| Behcet's disease | autoimmune |
| Bullous pemphigoid | autoimmune |
| Cardiomyopathy | autoimmune |
| Castleman disease | autoimmune |
| Celiac disease | autoimmune |
| Chagas disease | autoimmune |
| Chronic fatigue syndrome | autoimmune |
| Chronic inflammatory demyelinating polyneuropathy | autoimmune |
| (CIDP) | |
| Chronic recurrent multifocal ostomyelitis (CRMO) | autoimmune |
| Churg-Strauss syndrome | autoimmune |
| Cicatricial pemphigoid/benign mucosal pemphigoid | autoimmune |
| Crohn's disease | autoimmune |
| Cogans syndrome | autoimmune |
| Cold agglutinin disease | autoimmune |
| Congenital heart block | autoimmune |
| Coxsackie myocarditis | autoimmune |
| CREST disease | autoimmune |
| Essential mixed cryoglobulinemia | autoimmune |
| Demyelinating neuropathies | autoimmune |
| Dermatitis herpetiformis | autoimmune |
| Dermatomyositis | autoimmune |
| Devic's disease (neuromyelitis optica) | autoimmune |
| Discoid lupus | autoimmune |
| Dressler's syndrome | autoimmune |
| Endometriosis | autoimmune |
| Eosinophilic esophagitis | autoimmune |
| Eosinophilic fasciitis | autoimmune |
| Erythema nodosum | autoimmune |
| Experimental allergic encephalomyelitis | autoimmune |
| Evans syndrome | autoimmune |
| Fibromyalgia | autoimmune |
| Fibrosing alveolitis | autoimmune |
| Giant cell arteritis (temporal arteritis) | autoimmune |
| Giant cell myocarditis | autoimmune |
| Glomerulonephritis | autoimmune |
| Goodpasture's syndrome | autoimmune |
| Granulomatosis with Polyangiitis (GPA) (formerly called | autoimmune |
| Wegener's Granulomatosis) | |
| Graves' disease | autoimmune |
| Guillain-Barre syndrome | autoimmune |
| Hashimoto's encephalitis | autoimmune |
| Hashimoto's thyroiditis | autoimmune |
| Hemolytic anemia | autoimmune |
| Henoch-Schonlein purpura | autoimmune |
| Herpes gestationis | autoimmune |
| Hypogammaglobulinemia | autoimmune |
| Idiopathic thrombocytopenic purpura (ITP) | autoimmune |
| IgA nephropathy | autoimmune |
| IgG4-related sclerosing disease | autoimmune |
| Immunoregulatory lipoproteins | autoimmune |
| Inclusion body myositis | autoimmune |
| Interstitial cystitis | autoimmune |
| Juvenile arthritis | autoimmune |
| Juvenile diabetes (Type 1 diabetes) | autoimmune |
| Juvenile myositis | autoimmune |
| Kawasaki syndrome | autoimmune |
| Lambert-Eaton syndrome | autoimmune |
| Leukocytoclastic vasculitis | autoimmune |
| Lichen planus | autoimmune |
| Lichen sclerosis | autoimmune |
| Ligneous conjunctivitis | autoimmune |
| Linear IgA disease (LAD) | autoimmune |
| Lupus (SLE) | autoimmune |
| Lyme disease | autoimmune |
| chronic | autoimmune |
| Meniere's disease | autoimmune |
| Microscopic polyangiitis | autoimmune |
| Mixed connective tissue disease (MCTD) | autoimmune |
| Mooren's ulcer | autoimmune |
| Mucha-Habermann disease | autoimmune |
| Multiple sclerosis | autoimmune |
| Myasthenia gravis | autoimmune |
| Myositis | autoimmune |
| Narcolepsy | autoimmune |
| Neuromyelitis optica (Devic's) | autoimmune |
| Neutropenia | autoimmune |
| Ocular cicatricial pemphigoid | autoimmune |
| Optic neuritis | autoimmune |
| Palindromic rheumatism | autoimmune |
| PANDAS (Pediatric Autoimmune Neuropsychiatric | autoimmune |
| Disorders Associated with Streptococcus) | |
| Paraneoplastic cerebellar degeneration | autoimmune |
| Paroxysmal nocturnal hemoglobinuria (PNH) | autoimmune |
| Parry Romberg syndrome | autoimmune |
| Parsonnage-Turner syndrome | autoimmune |
| Pars planitis (peripheral uveitis) | autoimmune |
| Pemphigus | autoimmune |
| Peripheral neuropathy | autoimmune |
| Perivenous encephalomyelitis | autoimmune |
| Pernicious anemia | autoimmune |
| POEMS syndrome | autoimmune |
| Polyarteritis nodosa | autoimmune |
| Sjogren's syndrome | autoimmune |
| Type I autoimmune polyglandular syndromes | autoimmune |
| Type II autoimmune polyglandular syndromes | autoimmune |
| Type III autoimmune polyglandular syndromes | autoimmune |
| Polymyalgia rheumatica | autoimmune |
| Polymyositis | autoimmune |
| Postmyocardial infarction syndrome | autoimmune |
| Postpericardiotomy syndrome | autoimmune |
| Progesterone dermatitis | autoimmune |
| Primary biliary cirrhosis | autoimmune |
| Primary sclerosing cholangitis | autoimmune |
| Psoriasis | autoimmune |
| Psoriatic arthritis | autoimmune |
| Idiopathic pulmonary fibrosis | autoimmune |
| Pyoderma gangrenosum | autoimmune |
| Pure red cell aplasia | autoimmune |
| Raynauds phenomenon | autoimmune |
| Reactive Arthritis | autoimmune |
| Reflex sympathetic dystrophy | autoimmune |
| Reiter's syndrome | autoimmune |
| Relapsing polychondritis | autoimmune |
| Restless legs syndrome | autoimmune |
| Retroperitoneal fibrosis | autoimmune |
| Rheumatic fever | autoimmune |
| Rheumatoid arthritis | autoimmune |
| Sarcoidosis | autoimmune |
| Schmidt syndrome | autoimmune |
| Scleritis | autoimmune |
| Scleroderma | autoimmune |
| Sjogren's syndrome | autoimmune |
| Sperm & testicular autoimmunity | autoimmune |
| Stiff person syndrome | autoimmune |
| Subacute bacterial endocarditis (SBE) | autoimmune |
| Susac's syndrome | autoimmune |
| Sympathetic ophthalmia | autoimmune |
| Takayasu's arteritis | autoimmune |
| Temporal arteritis/Giant cell arteritis | autoimmune |
| Thrombocytopenic purpura (TTP) | autoimmune |
| Tolosa-Hunt syndrome | autoimmune |
| Transverse myelitis | autoimmune |
| Ulcerative colitis | autoimmune |
| Undifferentiated connective tissue disease (UCTD) | autoimmune |
| Uveitis | autoimmune |
| Vasculitis | autoimmune |
| Vesiculobullous dermatosis | autoimmune |
| Vitiligo | autoimmune |
| graft versus host disease (GVDH) | autoimmune |
| transplant rejection | autoimmune |
| lymphomas | cancer-related |
| leukemias | cancer-related |
| carcinomas | cancer-related |
| sarcomas | cancer-related |
| anal cancer | cancer-related |
| bladder cancer | cancer-related |
| bile duct cancer | cancer-related |
| bone cancer | cancer-related |
| brain cancer | cancer-related |
| breast cancer | cancer-related |
| cervical cancer | cancer-related |
| colon/rectum cancer | cancer-related |
| endometrial cancer | cancer-related |
| esophageal cancer | cancer-related |
| eye cancer | cancer-related |
| gallbladder cancer | cancer-related |
| head and neck cancer | cancer-related |
| liver cancer | cancer-related |
| kidney cancer | cancer-related |
| larynx cancer | cancer-related |
| lung cancer | cancer-related |
| mediastinum (chest) cancer | cancer-related |
| mouth cancer | cancer-related |
| ovarian cancer | cancer-related |
| pancreatic cancer | cancer-related |
| penile cancer | cancer-related |
| prostate cancer | cancer-related |
| skin cancer | cancer-related |
| intestinal cancer | cancer-related |
| stomach cancer | cancer-related |
| spinal marrow cancer | cancer-related |
| tailbone cancer | cancer-related |
| testicular cancer | cancer-related |
| thyroid cancer | cancer-related |
| uterine cancer | cancer-related |
| papilloma/carcinoma | cancer-related |
| choriocarcinoma | cancer-related |
| endodermal sinus tumor | cancer-related |
| teratoma | cancer-related |
| adenoma/adenocarcinoma | cancer-related |
| melanoma | cancer-related |
| fibroma | cancer-related |
| lipoma | cancer-related |
| leiomyoma | cancer-related |
| rhabdomyoma | cancer-related |
| mesothelioma | cancer-related |
| angioma | cancer-related |
| osteoma | cancer-related |
| chondroma | cancer-related |
| glioma | cancer-related |
| lymphoma/leukemia | cancer-related |
| squamous cell carcinoma | cancer-related |
| small cell carcinoma | cancer-related |
| large cell undifferentiated carcinomas | cancer-related |
| basal cell carcinoma | cancer-related |
| sinonasal undifferentiated carcinoma | cancer-related |
| soft tissue sarcoma | cancer-related |
| alveolar soft part sarcoma | cancer-related |
| angiosarcoma | cancer-related |
| dermatofibrosarcoma | cancer-related |
| desmoid tumor | cancer-related |
| desmoplastic small round cell tumor | cancer-related |
| extraskeletal chondrosarcoma | cancer-related |
| extraskeletal osteosarcoma | cancer-related |
| fibrosarcoma | cancer-related |
| hemangiopericytoma | cancer-related |
| hemangiosarcoma | cancer-related |
| Kaposi's sarcoma | cancer-related |
| leiomyosarcoma | cancer-related |
| liposarcoma | cancer-related |
| lymphangiosarcoma | cancer-related |
| lymphosarcoma | cancer-related |
| malignant fibrous histiocytoma | cancer-related |
| neurofibrosarcoma | cancer-related |
| rhabdomyosarcoma | cancer-related |
| synovial sarcoma | cancer-related |
| Askin's tumor | cancer-related |
| Ewing's sarcoma (primitive neuroectodermal tumor) | cancer-related |
| malignant hemangioendothelioma | cancer-related |
| malignant schwannoma | cancer-related |
| osteosarcoma | cancer-related |
| chondrosarcoma | cancer-related |
| Acute granulocytic leukemia | cancer-related |
| Acute lymphocytic leukemia | cancer-related |
| Acute myelogenous leukemia | cancer-related |
| Adenocarcinoma | cancer-related |
| Adenosarcoma | cancer-related |
| Adrenal cancer | cancer-related |
| Adrenocortical carcinoma | cancer-related |
| Anal cancer | cancer-related |
| Anaplastic astrocytoma | cancer-related |
| Angiosarcoma | cancer-related |
| Appendix cancer | cancer-related |
| Astrocytoma | cancer-related |
| Basal cell carcinoma | cancer-related |
| B-Cell lymphoma) | cancer-related |
| Bile duct cancer | cancer-related |
| Bladder cancer | cancer-related |
| Bone cancer | cancer-related |
| Bowel cancer | cancer-related |
| Brain cancer | cancer-related |
| Brain stem glioma | cancer-related |
| Brain tumor | cancer-related |
| Breast cancer | cancer-related |
| Carcinoid tumors | cancer-related |
| Cervical cancer | cancer-related |
| Cholangiocarcinoma | cancer-related |
| Chondrosarcoma | cancer-related |
| Chronic lymphocytic leukemia | cancer-related |
| Chronic myelogenous leukemia | cancer-related |
| Colon cancer | cancer-related |
| Colorectal cancer | cancer-related |
| Craniopharyngioma | cancer-related |
| Cutaneous lymphoma | cancer-related |
| Cutaneous melanoma | cancer-related |
| Diffuse astrocytoma | cancer-related |
| Ductal carcinoma in situ | cancer-related |
| Endometrial cancer | cancer-related |
| Ependymoma | cancer-related |
| Epithelioid sarcoma | cancer-related |
| Esophageal cancer | cancer-related |
| Ewing sarcoma | cancer-related |
| Extrahepatic bile duct cancer | cancer-related |
| Eye cancer | cancer-related |
| Fallopian tube cancer | cancer-related |
| Fibrosarcoma | cancer-related |
| Gallbladder cancer | cancer-related |
| Gastric cancer | cancer-related |
| Gastrointestinal cancer | cancer-related |
| Gastrointestinal carcinoid cancer | cancer-related |
| Gastrointestinal stromal tumors | cancer-related |
| General | cancer-related |
| Germ cell tumor | cancer-related |
| Glioblastoma multiforme | cancer-related |
| Glioma | cancer-related |
| Hairy cell leukemia | cancer-related |
| Head and neck cancer | cancer-related |
| Hemangioendothelioma | cancer-related |
| Hodgkin lymphoma | cancer-related |
| Hodgkin's disease | cancer-related |
| Hodgkin's lymphoma | cancer-related |
| Hypopharyngeal cancer | cancer-related |
| Infiltrating ductal carcinoma | cancer-related |
| Infiltrating lobular carcinoma | cancer-related |
| Inflammatory breast cancer | cancer-related |
| Intestinal Cancer | cancer-related |
| Intrahepatic bile duct cancer | cancer-related |
| Invasive/infiltrating breast cancer | cancer-related |
| Islet cell cancer | cancer-related |
| Jaw cancer | cancer-related |
| Kaposi sarcoma | cancer-related |
| Kidney cancer | cancer-related |
| Laryngeal cancer | cancer-related |
| Leiomyosarcoma | cancer-related |
| Leptomeningeal metastases | cancer-related |
| Leukemia | cancer-related |
| Lip cancer | cancer-related |
| Liposarcoma | cancer-related |
| Liver cancer | cancer-related |
| Lobular carcinoma in situ | cancer-related |
| Low-grade astrocytoma | cancer-related |
| Lung cancer | cancer-related |
| Lymph node cancer | cancer-related |
| Lymphoma | cancer-related |
| Male breast cancer | cancer-related |
| Medullary carcinoma | cancer-related |
| Medulloblastoma | cancer-related |
| Melanoma | cancer-related |
| Meningioma | cancer-related |
| Merkel cell carcinoma | cancer-related |
| Mesenchymal chondrosarcoma | cancer-related |
| Mesenchymous | cancer-related |
| Mesothelioma | cancer-related |
| Metastatic breast cancer | cancer-related |
| Metastatic melanoma | cancer-related |
| Metastatic squamous neck cancer | cancer-related |
| Mixed gliomas | cancer-related |
| Mouth cancer | cancer-related |
| Mucinous carcinoma | cancer-related |
| Mucosal melanoma | cancer-related |
| Multiple myeloma | cancer-related |
| Nasal cavity cancer | cancer-related |
| Nasopharyngeal cancer | cancer-related |
| Neck cancer | cancer-related |
| Neuroblastoma | cancer-related |
| Neuroendocrine tumors | cancer-related |
| Non-Hodgkin lymphoma | cancer-related |
| Non-Hodgkin's lymphoma | cancer-related |
| Non-small cell lung cancer | cancer-related |
| Oat cell cancer | cancer-related |
| Ocular cancer | cancer-related |
| Ocular melanoma | cancer-related |
| Oligodendroglioma | cancer-related |
| Oral cancer | cancer-related |
| Oral cavity cancer | cancer-related |
| Oropharyngeal cancer | cancer-related |
| Osteogenic sarcoma | cancer-related |
| Osteosarcoma | cancer-related |
| Ovarian cancer | cancer-related |
| Ovarian epithelial cancer | cancer-related |
| Ovarian germ cell tumor | cancer-related |
| Ovarian primary peritoneal carcinoma | cancer-related |
| Ovarian sex cord stromal tumor | cancer-related |
| Paget's disease | cancer-related |
| Pancreatic cancer | cancer-related |
| Papillary carcinoma | cancer-related |
| Paranasal sinus cancer | cancer-related |
| Parathyroid cancer | cancer-related |
| Pelvic cancer | cancer-related |
| Penile cancer | cancer-related |
| Peripheral nerve cancer | cancer-related |
| Peritoneal cancer | cancer-related |
| Pharyngeal cancer | cancer-related |
| Pheochromocytoma | cancer-related |
| Pilocytic astrocytoma | cancer-related |
| Pineal region tumor | cancer-related |
| Pineoblastoma | cancer-related |
| Pituitary gland cancer | cancer-related |
| Primary central nervous system lymphoma | cancer-related |
| Prostate cancer | cancer-related |
| Rectal cancer | cancer-related |
| Renal cell cancer | cancer-related |
| Renal pelvis cancer | cancer-related |
| Rhabdomyosarcoma | cancer-related |
| Salivary gland cancer | cancer-related |
| Sarcoma | cancer-related |
| Sarcoma | cancer-related |
| bone | cancer-related |
| Sarcoma | cancer-related |
| soft tissue | cancer-related |
| Sarcoma | cancer-related |
| uterine | cancer-related |
| Sinus cancer | cancer-related |
| Skin cancer | cancer-related |
| Small cell lung cancer | cancer-related |
| Small intestine cancer | cancer-related |
| Soft tissue sarcoma | cancer-related |
| Spinal cancer | cancer-related |
| Spinal column cancer | cancer-related |
| Spinal cord cancer | cancer-related |
| Spinal tumor | cancer-related |
| Squamous cell carcinoma | cancer-related |
| Stomach cancer | cancer-related |
| Synovial sarcoma | cancer-related |
| T-cell lymphoma) | cancer-related |
| Testicular cancer | cancer-related |
| Throat cancer | cancer-related |
| Thymoma/thymic carcinoma | cancer-related |
| Thyroid cancer | cancer-related |
| Tongue cancer | cancer-related |
| Tonsil cancer | cancer-related |
| Transitional cell cancer | cancer-related |
| Transitional cell cancer | cancer-related |
| Transitional cell cancer | cancer-related |
| Triple-negative breast cancer | cancer-related |
| Tubal cancer | cancer-related |
| Tubular carcinoma | cancer-related |
| Ureteral cancer | cancer-related |
| Ureteral cancer | cancer-related |
| Urethral cancer | cancer-related |
| Uterine adenocarcinoma | cancer-related |
| Uterine cancer | cancer-related |
| Uterine sarcoma | cancer-related |
| Vaginal cancer | cancer-related |
| Vulvar cancer | cancer-related |
| lysosomal storage disease | CNS |
| mental retardation | CNS |
| seizures | CNS |
| profound neurodegeneration | CNS |
| behavioral abnormalities | CNS |
| psycho-motor defects | CNS |
| Mucopolysaccharidosis type II (Hunter Syndrome | CNS |
| iduronate sulfatase deficiency) | CNS |
| Mucopolysaccharidosis type VI (Maroteaux-Lamy | CNS |
| Syndrome | |
| arylsulfatase B deficiency) | CNS |
| Mucopolysaccharidosis type III (Sanfilippo A) | CNS |
| Mucopolysaccharidosis type IV (MPS IV) | CNS |
| Pompe disease (acid maltase deficiency) | CNS |
| Niemann-Pick B (NP-B) disease | CNS |
| metachromatic leukodystrophy (MLD | CNS |
| Arylsufatase A deficiency) | CNS |
| Krabbe disease | CNS |
| Wolman disease | CNS |
| Sly syndrome | CNS |
| degenerative disease | CNS |
| Alzheimer's disease (AD) | CNS |
| Huntington's Disease (HD) | CNS |
| Parkinson's disease (PD) | CNS |
| Psittacosis | general |
| Ritter's Disease | general |
| 14-day measles | general |
| 7-day fever | general |
| Acne | general |
| Acrodermatitis chronica atrophicans (ACA)- (late skin | general |
| manifestation of latent Lyme disease) | |
| Acute bacterial rhinosinusitis | general |
| Acute hemorrhagic conjunctivitis | general |
| Acute hemorrhagic cystitis | general |
| Acute rhinosinusitis | general |
| Adult T-cell Leukemia-Lymphoma (ATLL) | general |
| African Sleeping Sickness | general |
| AIDS (Acquired Immunodeficiency Syndrome) | general |
| Alveolar hydatid | general |
| Amebiasis | general |
| Amebic meningoencephalitis | general |
| Anaplasmosis | general |
| Anthrax | general |
| Arboviral or parainfectious disease | general |
| Ascariasis | general |
| Aseptic meningitis | general |
| Athlete's foot (Tinea pedis) | general |
| Australian tick typhus | general |
| Avian Influenza | general |
| Babesiosis | general |
| Bacillary angiomatosis | general |
| bacterial | general |
| Bacterial meningitis | general |
| Bacterial vaginosis | general |
| Balanitis | general |
| Balantidiasis | general |
| Bang's disease | general |
| Barmah Forest virus infection | general |
| Bartonellosis | general |
| Bat Lyssavirus Infection | general |
| Bay sore (Chiclero's ulcer) | general |
| Baylisascaris infection (Racoon roundworm infection) | general |
| Beaver fever | general |
| Beef tapeworm | general |
| Bejel (endemic syphilis) | general |
| Biphasic meningoencephalitis | general |
| Black Bane | general |
| Black death | general |
| Black piedra | general |
| Blackwater Fever | general |
| Blastomycosis | general |
| Blennorrhea of the newborn | general |
| Blepharitis | general |
| Boils | general |
| Bordetella paratussis | general |
| Bordetella pertussis | general |
| Bornholm disease (pleurodynia) | general |
| Borrelia miyamotoi Disease | general |
| Botulism | general |
| Boutonneuse fever | general |
| Brazilian purpuric fever | general |
| Break Bone fever | general |
| Brill | general |
| Bronchiolitis | general |
| Bronchitis | general |
| Brucellosis (Bang's disease) | general |
| Bubonic plague | general |
| Bullous impetigo | general |
| Burkholderia mallei (Glanders) | general |
| Burkholderia pseudomallei (Melioidosis) | general |
| Buruli ulcers | general |
| Busse | general |
| Busse-Buschke disease (Cryptococcosis) | general |
| California group encephalitis | general |
| Campylobacteriosis | general |
| Candida | general |
| Candida albicans | general |
| Candida glabrata | general |
| Candida krusei | general |
| Candida lusitaniae | general |
| Candida parapsilosis | general |
| Candida tropicalis | general |
| Candidiasis | general |
| Canefield fever | general |
| canefield fever | general |
| Canicola fever | general |
| Canicola fever | general |
| Capillariasis | general |
| Carate | general |
| Carbapenem-resistant Enterobacteriaceae (CRE) | general |
| Carbuncle | general |
| Carrion's disease | general |
| Carrion's disease | general |
| Cat Scratch fever | general |
| Cave disease | general |
| Central Asian hemorrhagic fever | general |
| Central European tick | general |
| Cervical cancer | general |
| Chagas disease | general |
| Chancroid (Soft chancre) | general |
| Chicago disease | general |
| Chickenpox (Varicella) | general |
| Chiclero's ulcer | general |
| Chikungunya fever | general |
| Chlamydial infection | general |
| Cholera | general |
| Chromoblastomycosis | general |
| Ciguatera | general |
| Clap | general |
| Clonorchiasis (Liver fluke infection) | general |
| Clostridium Difficile Infection | general |
| ClostriDium Perfringens (Epsilon Toxin) | general |
| Clostridium Tetani | general |
| Coccidioidomycosis fungal infection | general |
| Coenurosis | general |
| Colorado tick fever | general |
| Condyloma accuminata | general |
| Condyloma accuminata (Warts) | general |
| Condyloma lata | general |
| Congenital impetigo | general |
| Congo fever | general |
| Congo hemorrhagic fever virus | general |
| Conjunctivitis | general |
| Coronaviruses | general |
| cowpox | general |
| Crabs | general |
| Crimean | general |
| Croup | general |
| Cryptococcosis | general |
| Cryptosporidiosis (Crypto) | general |
| Cutaneous Larval Migrans | general |
| Cyclosporiasis | general |
| Cystic hydatid | general |
| Cysticercosis | general |
| Cystitis | general |
| Czechoslovak tick | general |
| D68 (EV-D68) | general |
| Dacryocytitis | general |
| Dandy fever | general |
| Darling's Disease | general |
| Deer fly fever | general |
| Dengue fever (1, 2, 3, or 4) | general |
| desert rheumatism | general |
| Desert rheumatism | general |
| Devil's grip | general |
| Diphasic milk fever | general |
| Diphtheria | general |
| Disseminated Intravascular Coagulation | general |
| Dog tapeworm | general |
| Donovanosis | general |
| Donovanosis (Granuloma inguinale) | general |
| Dracontiasis | general |
| Dracunculosis | general |
| Duke's disease | general |
| Dum Dum Disease | general |
| Durand-Nicholas-Favre disease | general |
| Dwarf tapeworm | general |
| E. Coli infection (E. Coli) | general |
| Eastern equine encephalitis | general |
| Ebola Hemorrhagic Fever (Ebola virus disease EVD) | general |
| Ectothrix | general |
| Ehrlichiosis (Sennetsu fever) | general |
| Encephalitis | general |
| Endemic Relapsing fever | general |
| Endemic syphilis | general |
| Endophthalmitis | general |
| Endothrix | general |
| Enterobiasis (Pinworm infection) | general |
| Enterotoxin - B Poisoning (Staph Food Poisoning) | general |
| Enterovirus 71 | general |
| Enterovirus Infection | general |
| Epidemic Keratoconjunctivitis | general |
| Epidemic Relapsing fever | general |
| Epidemic typhus | general |
| Epiglottitis | general |
| Epstein-Barr virus | general |
| Erysipelis | general |
| Erysipeloid (Erysipelothricosis) | general |
| Erythema chronicum migrans | general |
| Erythema infectiosum | general |
| Erythema marginatum | general |
| Erythema multiforme | general |
| Erythema nodosum | general |
| Erythema nodosum leprosum | general |
| Erythrasma | general |
| Espundia | general |
| Eumycotic mycetoma | general |
| European blastomycosis | general |
| Exanthem subitum (Sixth disease) | general |
| Eyeworm | general |
| Far Eastern tick | general |
| Fascioliasis | general |
| Fievre boutonneuse (Tick typhus) | general |
| Fifth Disease (erythema infectiosum) | general |
| Filatow-Dukes' Disease (Scalded Skin Syndrome | general |
| Fish tapeworm | general |
| Fitz-Hugh-Curtis syndrome | general |
| Flinders Island Spotted Fever | general |
| Flu (Influenza) | general |
| Folliculitis | general |
| Four Corners Disease (Human Pulmonary Syndrome | general |
| (HPS)) | |
| Frambesia | general |
| Francis disease | general |
| Fiancisella Tularensis | general |
| fungal | general |
| Furunculosis | general |
| Gas gangrene | general |
| Gastroenteritis | general |
| Genital Herpes | general |
| Genital Warts | general |
| German measles | general |
| German Measles | general |
| Gerstmann-Straussler-Scheinker (GSS) | general |
| Giardiasis | general |
| Gilchrist's disease | general |
| Gingivitis | general |
| Gingivostomatitis | general |
| Glanders | general |
| Glandular fever (infectious mononucleosis) | general |
| Gnathostomiasis | general |
| Gonococcal Infection (Gonorrhea) | general |
| Gonorrhea | general |
| Granuloma inguinale (Donovanosis) | general |
| Guinea Worm | general |
| Haemophilus Influenza disease | general |
| Hamburger disease | general |
| Hansen's disease - leprosy | general |
| Hantaan disease | general |
| Hantaan-Korean hemorrhagic fever | general |
| Hantavirus Pulmonary Syndrome | general |
| Hantavirus Pulmonary Syndrome (HPS) | general |
| Hard chancre | general |
| Hard measles | general |
| Haverhill fever | general |
| Head and Body Lice | general |
| Heartland fever | general |
| Helicobacterosis | general |
| Hemolytic Uremic Syndrome (HUS) | general |
| Hepatitis A | general |
| Hepatitis B | general |
| Hepatitis B | general |
| Hepatitis C | general |
| Hepatitis C | general |
| Hepatitis D | general |
| Hepatitis D | general |
| Hepatitis E | general |
| Herpangina | general |
| Herpes- genital | general |
| Herpes labialis | general |
| Herpes- neonatal | general |
| Herpes simplex virus 1 and 2 | general |
| Hidradenitis | general |
| Histoplasmosis | general |
| Histoplasmosis infection (Histoplasmosis) | general |
| His-Werner disease | general |
| HIV infection | general |
| Hookworm infections | general |
| Hordeola | general |
| Hordeola (Stye) | general |
| HTLV | general |
| HTLV- associated myelopathy (HAM) | general |
| Human Cytomegalovirus | general |
| Human granulocytic ehrlichiosis | general |
| Human immunodeficiency virus (HIV) | general |
| Human monocytic ehrlichiosis | general |
| Human papilloma virus | general |
| Human Papillomavirus (HPV) | general |
| Human Pulmonary Syndrome | general |
| Hydatid cyst | general |
| Hydrophobia | general |
| Impetigo | general |
| Inclusion conjunctivitis | general |
| Inclusion conjunctivitis - Swimming Pool conjunctivitis- | general |
| Infantile diarrhea | general |
| Infectious Mononucleosis | general |
| Infectious myocarditis | general |
| Infectious pericarditis | general |
| Inflammation | general |
| Influenza | general |
| Influenza virus | general |
| Isosporiasis | general |
| Israeli spotted fever | general |
| Japanese Encephalitis | general |
| Jock itch | general |
| John Cunningham Virus (JCV) | general |
| Jorge Lobo disease - lobomycosis | general |
| Jungle yellow fever | general |
| Junin Argentinian hemorrhagic fever | general |
| Kala Azar | general |
| Kaposi's sarcoma | general |
| Keloidal blastomycosis | general |
| Keratoconjunctivitis | general |
| Kuru | general |
| Kyasanur forest disease | general |
| LaCrosse encephalitis | general |
| Lassa hemorrhagic fever | general |
| Legionellosis (Legionnaires Disease) | general |
| Legionnaire's pneumonia | general |
| Lemierre's Syndrome (Postanginal septicemia) | general |
| Lemming fever | general |
| Leprosy | general |
| leptospirosis | general |
| Leptospirosis (Nanukayami fever | general |
| Listeriosis (Listeria) | general |
| Liver disease | general |
| Liver fluke infection | general |
| Lobo's mycosis | general |
| Lockjaw | general |
| Loiasis | general |
| Louping Ill | general |
| Ludwig's angina | general |
| Lung fluke infection | general |
| Lung fluke infection (Paragonimiasis) | general |
| Lyme disease | general |
| Lymphogranuloma venereum infection (LGV) | general |
| Machupo Bolivian hemorrhagic fever | general |
| Madura foot | general |
| Mal del pinto | general |
| Malaria | general |
| Malignant pustule | general |
| Malta fever | general |
| Marburg hemorrhagic fever | general |
| Masters disease | general |
| Maternal Sepsis (Puerperal fever) | general |
| Measles | general |
| Mediterranean spotted fever | general |
| Melioidosis (Whitmore's disease) | general |
| Meningitis | general |
| Meningococcal Disease | general |
| MERS | general |
| Methicillin-resistant Staphylococcus aureus (MRSA) | general |
| Milker's nodule | general |
| Molluscum contagiosum | general |
| Moniliasis | general |
| monkeypox | general |
| Mononucleosis | general |
| Mononucleosis-like syndrome | general |
| Montezuma's Revenge | general |
| Morbilli | general |
| MRSA (methicillin-resistant Staphylococcus aureus) | general |
| infection | |
| Mucormycosis- Zygomycosis | general |
| Multiple Organ Dysfunction Syndrome or MODS | general |
| Multiple-system atrophy (MSA) | general |
| Mumps | general |
| Murine typhus | general |
| Murray Valley Encephalitis(MVE) | general |
| Mycobacterium | general |
| Mycoburuli ulcers | general |
| Mycoburuli ulcers | general |
| Mycoburuli ulcers- Buruli ulcers | general |
| Mycotic vulvovaginitis | general |
| Myositis | general |
| Nanukayami fever | general |
| Necrotizing fasciitis | general |
| Necrotizing fasciitis- Type 1 | general |
| Necrotizing fasciitis- Type 2 | general |
| Negishi | general |
| New world spotted fever | general |
| Nocardiosis | general |
| Nongonococcal urethritis | general |
| Non-Polio (Non-Polio Enterovirus) | general |
| Norovirus infection | general |
| North American blastomycosis | general |
| North Asian tick typhus | general |
| Norwalk virus infection | general |
| Norwegian itch | general |
| O'Hara disease | general |
| Omsk hemorrhagic fever | general |
| Onchoceriasis | general |
| Onychomycosis | general |
| Opisthorchiasis | general |
| Opthalmia neonatorium | general |
| Oral hairy leukoplakia | general |
| Orf | general |
| Organ injury | general |
| Oriental Sore | general |
| Oriental Spotted Fever | general |
| Ornithosis (Parrot fever | general |
| Oroya fever | general |
| Oroya fever | general |
| Otitis externa | general |
| Otitis media | general |
| Pannus | general |
| Pannus | general |
| Paracoccidioidomycosis | general |
| Paragonimiasis | general |
| Paralytic Shellfish Poisoning (Paralytic Shellfish | general |
| Poisoning) | |
| parasitic | general |
| Paronychia (Whitlow) | general |
| Parotitis | general |
| PCP pneumonia | general |
| Pediculosis | general |
| Peliosis hepatica | general |
| Pelvic Inflammatory Disease | general |
| Perihepatitis | general |
| Pertussis (also called Whooping cough) | general |
| Phaeohyphomycosis | general |
| Pharyngoconjunctival fever | general |
| Piedra (White Piedra) | general |
| Piedra (Black Piedra) | general |
| Pigbel | general |
| Pink eye conjunctivitis | general |
| Pinta | general |
| Pinworm infection | general |
| Type I diabetes | general |
| Type II diabetes | general |
| gestational diabetes | general |
| latent autoimmune diabetes | general |
| Pitted Keratolysis | general |
| Pityriasis versicolor (Tinea versicolor) | general |
| Plague | general |
| Pleurodynia | general |
| Pneumococcal Disease | general |
| Pneumocystosis | general |
| Pneumonia | general |
| Pneumonic (Plague) | general |
| Polio or Poliomyelitis | general |
| Polycystic hydatid | general |
| Pontiac fever | general |
| Pork tapeworm | general |
| Posada-Wernicke disease | general |
| Postanginal septicemia | general |
| Powassan | general |
| Poxviruses | general |
| Progressive multifocal leukencephalopathy | general |
| Progressive Rubella Panencephalitis | general |
| Prostatitis | general |
| Pseudomembranous colitis | general |
| Psittacosis | general |
| Puerperal fever | general |
| Pustular Rash diseases (Small pox) | general |
| Pyelonephritis | general |
| Pylephlebitis | general |
| Q-Fever | general |
| Quinsy | general |
| Quintana fever (5-day fever) | general |
| Rabbit fever | general |
| Rabies | general |
| Racoon roundworm infection | general |
| Rat bite fever | general |
| Rat bite fever | general |
| Rat tapeworm | general |
| Reiter Syndrome | general |
| Relapsing fever | general |
| Respiratory syncytial virus (RSV) | general |
| Respiratory syncytial virus (RSV) infection | general |
| Rheumatic fever | general |
| Rhodotorulosis | general |
| Ricin Poisoning | general |
| Rickettsialpox | general |
| Rickettsiosis | general |
| Rift Valley Fever | general |
| Ringworm | general |
| Ritter's Disease | general |
| River Blindness | general |
| Rocky Mountain spotted fever | general |
| Rose Handler's disease (Sporotrichosis) | general |
| Rose rash of infants | general |
| Roseola | general |
| Ross River fever | general |
| Rotavirus infection | general |
| Roundworm infection | general |
| Roundworm infections | general |
| Rubella | general |
| Rubella virus | general |
| Rubeola | general |
| Russian spring | general |
| Salmonellosis gastroenteritis | general |
| San Joaquin Valley fever | general |
| Sao Paulo Encephalitis | general |
| Sao Paulo fever | general |
| SARS | general |
| Scabies Infestation (Scabies) (Norwegian itch) | general |
| Scalded Skin Syndrome | general |
| Scarlet fever (Scarlatina) | general |
| Schistosomiasis | general |
| Scombroid | general |
| Scrub typhus | general |
| Sennetsu fever | general |
| Sepsis (Septic shock) | general |
| Severe Acute Respiratory Syndrome | general |
| Severe Acute Respiratory Syndrome (SARS) | general |
| Shiga Toxigenic Escherichia coli (STEC/VTEC) | general |
| Shigellosis gastroenteritis (Shigella) | general |
| Shinbone fever | general |
| Shingles | general |
| Shipping fever | general |
| Siberian tick typhus | general |
| Sinusitis | general |
| Sixth disease | general |
| Slapped cheek disease | general |
| Sleeping sickness | general |
| Smallpox (Variola) | general |
| Snail Fever | general |
| Soft chancre | general |
| Southern tick associated rash illness | general |
| Sparganosis | general |
| Spelunker's disease | general |
| Sporadic typhus | general |
| Sporotrichosis | general |
| Spotted fever | general |
| Spring | general |
| Lyme disease | general |
| St. Louis encephalitis | general |
| Staphylococcal Food Poisoning | general |
| Staphylococcal Infection | general |
| Staphylococcus aureus (S. aureus) | general |
| Staphylococcus epidermidis (S. epidermidis) | general |
| Strep throat | general |
| Streptococcal Disease | general |
| Streptococcal Toxic-Shock Syndrome | general |
| Streptococcus pneumoniae | general |
| Streptococcus viridans | general |
| Strongyloiciasis | general |
| Stye | general |
| Subacute Sclerosing Panencephilitis | general |
| Subacute Sclerosing Panencephalitis (SSPE) | general |
| Sudden Acute Respiratory Syndrome | general |
| Sudden Rash | general |
| Swimmer's ear | general |
| Swimmer's Itch | general |
| Swimming Pool conjunctivitis | general |
| Sylvatic yellow fever | general |
| Syphilis | general |
| Systemic Inflammatory Response Syndrome (SIRS) | general |
| Tabes dorsalis (tertiary syphilis) | general |
| Taeniasis | general |
| Taiga encephalitis | general |
| Tanner's disease | general |
| Tapeworm infections | general |
| Temporal lobe encephalitis | general |
| Temporal lobe encephalitis | general |
| tetani (Lock Jaw) | general |
| Tetanus Infection | general |
| Threadworm infections | general |
| Thrush | general |
| Tick | general |
| Tick typhus | general |
| Tinea barbae | general |
| Tinea capitis | general |
| Tinea corporis | general |
| Tinea cruris | general |
| Tinea manuum | general |
| Tinea nigra | general |
| Tinea pedis | general |
| Tinea unguium | general |
| Tinea versicolor | general |
| Torulopsosis | general |
| Torulosis | general |
| Toxic Shock Syndrome | general |
| Toxoplasma gondii | general |
| Toxoplasmosis | general |
| transmissible spongioform (CJD) | general |
| Traveler's diarrhea | general |
| Trench fever 5 | general |
| Trichinellosis | general |
| Trichomoniasis | general |
| Trichomycosis axillaris | general |
| Trichuriasis | general |
| Tropical Spastic Paraparesis (TSP) | general |
| Trypanosomiasis | general |
| Tuberculosis (TB) | general |
| Tuberculosis | general |
| Tularemia | general |
| Typhoid Fever | general |
| Typhus fever | general |
| Ulcus molle | general |
| Undulant fever | general |
| Urban yellow fever | general |
| Urethritis | general |
| Vaginitis | general |
| Vaginosis | general |
| Valley fever | general |
| Vancomycin Intermediate (VISA) | general |
| Vancomycin Resistant (VRSA) | general |
| Vancomycin-intermediate Staphylococcus aureus (VISA) | general |
| Vancomycin-resistant Staphylococcus aureus (VRSA) | general |
| Varicella | general |
| Varicella zoster virus | general |
| Venezuelan Equine encephalitis | general |
| Verruga peruana | general |
| Verruga peruana | general |
| Vibrio cholerae (Cholera) | general |
| Vibriosis (Vibrio) | general |
| Vincent's disease or Trench mouth | general |
| viral | general |
| Viral conjunctivitis | general |
| Viral Meningitis | general |
| Viral meningoencephalitis | general |
| Viral rash | general |
| Visceral Larval Migrans | general |
| Vomito negro | general |
| Vulvovaginitis | general |
| Warts | general |
| Waterhouse | general |
| Weil's disease | general |
| West Nile Fever | general |
| Western equine encephalitis | general |
| Whipple's disease | general |
| Whipworm infection | general |
| White Piedra | general |
| Whitlow | general |
| Whitmore's disease | general |
| Winter diarrhea | general |
| Wolhynia fever | general |
| Wool sorters' disease | general |
| Yaws | general |
| Yellow Fever | general |
| Yersinosis | general |
| Yersinosis (Yersinia) | general |
| Zahorsky's disease | general |
| Zika virus disease | general |
| Zoster | general |
| Zygomycosis | general |
| dryness | general |
| dry eye disease | general |
| genetic disorder | genetic |
| Achondroplasia | genetic |
| Alpha-1 Antitrypsin Deficiency | genetic |
| Antiphospholipid Syndrome | genetic |
| Autism | genetic |
| Autosomal Dominant Polycystic Kidney Disease | genetic |
| Breast cancer | genetic |
| Charcot-Marie-Tooth | genetic |
| Colon cancer | genetic |
| Cri du chat | genetic |
| Crohn's Disease | genetic |
| Cystic fibrosis | genetic |
| Dercum Disease | genetic |
| Down Syndrome | genetic |
| Duane Syndrome | genetic |
| Duchenne Muscular Dystrophy | genetic |
| Factor V Leiden Thrombophilia | genetic |
| Familial Hypercholesterolemia | genetic |
| Familial Mediterranean Fever | genetic |
| Fragile X Syndrome | genetic |
| Gaucher Disease | genetic |
| Hemochromatosis | genetic |
| Hemophilia | genetic |
| Holoprosencephaly | genetic |
| Huntington's disease. Klinefelter syndrome | genetic |
| Marfan syndrome | genetic |
| Myotonic Dystrophy | genetic |
| Neurofibromatosis | genetic |
| Noonan Syndrome | genetic |
| Osteogenesis Imperfecta | genetic |
| Parkinson's disease | genetic |
| Phenylketonuria | genetic |
| Poland Anomaly | genetic |
| Porphyria | genetic |
| Progeria | genetic |
| Prostate Cancer | genetic |
| Retinitis Pigmentosa | genetic |
| Severe Combined Immunodeficiency (SCID) | genetic |
| Sickle cell disease | genetic |
| Skin Cancer | genetic |
| Spinal Muscular Atrophy | genetic |
| Tay-Sachs | genetic |
| Thalassemia | genetic |
| Trimethylaminuria | genetic |
| Turner Syndrome | genetic |
| Velocardiofacial Syndrome | genetic |
| WAGR Syndrome | genetic |
| Wilson Disease | genetic |
| coagulation disorder | genetic |
| hemophilia A (factor VIII deficiency) | genetic |
| hemophilia B | genetic |
| factor IX deficiency | genetic |
| Christmas disease | genetic |
| hemophilia C | genetic |
| factor XI deficiency | genetic |
| mild bleeding tendency | genetic |
| Von Willebrand disease | genetic |
| Bernard-Soulier syndrome | genetic |
| thrombophlebitis | genetic |
| Congenital afibrinogenemia | genetic |
| Familial renal amyloidosis | genetic |
| congenital proconvertin/factor VII deficiency | genetic |
| Thrombophilia | genetic |
| Congenital Factor X deficiency | genetic |
| Congenital Factor XIIIa/b deficiency | genetic |
| Prekallikrein/Fletcher Factor deficiency | genetic |
| Kininogen deficiency | genetic |
| Glomerulopathy with fibronectin deposits | genetic |
| Heparin cofactor II deficiency | genetic |
| Protein C deficiency | genetic |
| Protein S deficiency | genetic |
| Protein Z deficiency | genetic |
| Antithrombin III deficiency | genetic |
| Plasminogen deficiency | genetic |
| type I (ligneous conjunctivitis) | genetic |
| Antiplasmin deficiency | genetic |
| Plasminogen activator inhibitor-1 deficiency | genetic |
| Quebec platelet disorder | genetic |
| adenovirus | infectious |
| Anaplasma phagocytophilium | infectious |
| Ascaris lumbricoides | infectious |
| Bacillus anthracis | infectious |
| Bacillus cereus | infectious |
| Bacteriodes | infectious |
| Barmah Forest virus | infectious |
| Bartonella bacilliformis | infectious |
| Bartonella henselae | infectious |
| Bartonella quintana | infectious |
| beta-toxin of Clostridium perfringens | infectious |
| Bordetella pertussis | infectious |
| Bordetella parapertussis | infectious |
| Borrelia burgdorferi | infectious |
| Borrelia miyamotoi | infectious |
| Borrelia recurrentis | infectious |
| Borrelia sp. | infectious |
| Botulinum toxin | infectious |
| Brucella sp. | infectious |
| Burkholderia pseudomallei | infectious |
| California encephalitis virus | infectious |
| Campylobacter | infectious |
| Candida albicans | infectious |
| chikungunya virus | infectious |
| Chlamydia psittaci | infectious |
| Chlamydia trachomatis | infectious |
| Clonorchis sinensis | infectious |
| Clostridium difficile bacteria | infectious |
| Clostridium tetani | infectious |
| Colorado tick fever virus | infectious |
| Corynebacterium diphtheriae | infectious |
| Corynebacterium minutissimum | infectious |
| Coxiella burnetii | infectious |
| coxsackie A | infectious |
| coxsackie B | infectious |
| Crimean-Congo hemorrhagic fever virus | infectious |
| cytomegalovirus | infectious |
| dengue virus | infectious |
| Eastern Equine encephalitis virus | infectious |
| Ebola viruses | infectious |
| echovirus | infectious |
| Ehrlichia chaffeensis. | infectious |
| Ehrlichia equi. | infectious |
| Ehrlichia sp. | infectious |
| Entamoeba histolytica | infectious |
| Enterobacter sp. | infectious |
| Enterococcus faecalis | infectious |
| Enterovirus 71 | infectious |
| Epstein-Barr virus (EBV) | infectious |
| Erysipelothrix rhusiopathiae | infectious |
| Escherichia coli | infectious |
| Flavivirus | infectious |
| Fusobacterium necrophorum | infectious |
| Gardnerella vaginalis | infectious |
| Group B streptococcus | infectious |
| Haemophilus aegyptius | infectious |
| Haemophilus ducreyi | infectious |
| Haemophilus influenzae | infectious |
| hantavirus | infectious |
| Helicobacter pylori | infectious |
| Hepatitis A | infectious |
| Hepatitis B | infectious |
| Hepatitis C | infectious |
| Hepatitis D | infectious |
| Hepatitis E | infectious |
| herpes simplex virus 1 | infectious |
| herpes simplex virus 2 | infectious |
| human herpes virus 6 | infectious |
| human herpes Virus 8 | infectious |
| human immunodeficiency virus 1 | infectious |
| human immunodeficiency virus 2 | infectious |
| human T-cell leukemia virus I | infectious |
| human T-cell leukemia virus II | infectious |
| influenza viruses (A | infectious |
| B | infectious |
| C) | infectious |
| Jamestown Canyon virus | infectious |
| Japanese encephalitis antigenic | infectious |
| Japanese encephalitis virus | infectious |
| John Cunninham virus | infectious |
| juninvirus | infectious |
| Kaposi's Sarcoma-associated Herpes Virus (KSHV) | infectious |
| Klebsiella granulomatis | infectious |
| Klebsiella sp. | infectious |
| Kyasanur Forest Disease virus | infectious |
| La Crosse virus | infectious |
| Lassavirus | infectious |
| Legionella pneumophila | infectious |
| Leptospira interrogans | infectious |
| Listeria monocytogenes | infectious |
| lymphocytic choriomeningitis virus | infectious |
| lyssavirus | infectious |
| Machupovirus | infectious |
| Marburg virus | infectious |
| measles virus | infectious |
| MERS coronavirus (MERS-CoV) | infectious |
| Micrococcus sedentarius | infectious |
| Mobiluncus sp. | infectious |
| Moliuscipoxvirus | infectious |
| Moraxella catarrhalis | infectious |
| Morbilli- Rubeola virus | infectious |
| Mumpsvirus | infectious |
| Mycobacterium leprae | infectious |
| Mycobacterium tuberculosis | infectious |
| Mycobacterium ulceraus | infectious |
| Mycoplasma genitalium | infectious |
| Mycoplasma sp | infectious |
| Nairovirus, | infectious |
| Neisseria gonorrhoeae | infectious |
| Neisseria meningitidis | infectious |
| Nocardia | infectious |
| Norwalk virus | infectious |
| norovirus | infectious |
| Omsk hemorrhagic fever virus | infectious |
| papilloma virus | infectious |
| parainfluenza viruses 1-3 | infectious |
| parapoxvirus | infectious |
| parvovirus B19 | infectious |
| Peptostreptococccus sp. | infectious |
| Plasmodium sp. | infectious |
| polioviruses types I | infectious |
| II | infectious |
| and III | infectious |
| Proteus sp. | infectious |
| Pseudomonas aeruginosa | infectious |
| Pseudomonas pseudomallei | infectious |
| Pseudomonas sp. | infectious |
| rabies virus | infectious |
| respiratory syncytial virus | infectious |
| ricin toxin | infectious |
| Rickettsia australis | infectious |
| Rickettsia conori | infectious |
| Rickettsia honei | infectious |
| Rickettsia prowazekii | infectious |
| Ross River Virus | infectious |
| rotavirus | infectious |
| rubellavirus | infectious |
| Saint Louis encephalitis | infectious |
| Salmonella Typhi | infectious |
| Sarcoptes scabiei | infectious |
| SARS-associated coronavirus (SARS-CoV) | infectious |
| Serratia sp. | infectious |
| Shiga toxin and Shiga-like toxin | infectious |
| Shigella | infectious |
| Sin Nombre Virus | infectious |
| Snowshoe hare virus | infectious |
| Staphylococcus aureus | infectious |
| Staphylococcus epidermidis | infectious |
| Streptobacillus moniliformis | infectious |
| Streptococcus pneumoniae | infectious |
| Streptococcus agalactiae | infectious |
| Streptococcus agalactiae | infectious |
| Streptococcus group A-H | infectious |
| Streptococcus pneumoniae | infectious |
| Streptococcus pyogenes | infectious |
| Treponema pallidum subsp. Pallidum | infectious |
| Treponema pallidum var. carateum | infectious |
| Treponema pallidum var. endemicum | infectious |
| Tropheryma whippelii | infectious |
| Ureaplasma urealyticum | infectious |
| Varicella-Zoster virus | infectious |
| variola virus | infectious |
| Vibrio cholerae | infectious |
| West Nile virus | infectious |
| yellow fever virus | infectious |
| Yersinia enterocolitica | infectious |
| Yersinia pestis | infectious |
| Zika virus | infectious |
| joint disease | inflammatory |
| an ophthalmic disease | inflammatory |
| retinal disease | inflammatory |
| psoriasis | inflammatory |
| Crohn's disease | inflammatory |
| irritable bowel syndrome | inflammatory |
| Sjogren's disease | inflammatory |
| tissue graft rejection | inflammatory |
| asthma | inflammatory |
| systemic lupus erythematosus | inflammatory |
| glomerulonephritis | inflammatory |
| dermatomyositis | inflammatory |
| multiple sclerosis | inflammatory |
| scleroderma | inflammatory |
| vasculitis | inflammatory |
| Goodpasture's syndrome | inflammatory |
| atherosclerosis | inflammatory |
| chronic idiopathic thrombocytopenic purpura | inflammatory |
| Addison's disease | inflammatory |
| Parkinson's disease | inflammatory |
| Alzheimer's disease | inflammatory |
| diabetes | inflammatory |
| septic shock | inflammatory |
| myasthenia gravis | inflammatory |
| inflammatory pelvic disease | inflammatory |
| inflammatory bowel disease | inflammatory |
| urethritis | inflammatory |
| uveitis | inflammatory |
| sinusitis | inflammatory |
| pneumonitis | inflammatory |
| encephalitis | inflammatory |
| meningitis | inflammatory |
| myocarditis | inflammatory |
| nephritis | inflammatory |
| osteomyelitis | inflammatory |
| myositis | inflammatory |
| hepatitis | inflammatory |
| gastritis | inflammatory |
| enteritis | inflammatory |
| dermatitis | inflammatory |
| appendicitis | inflammatory |
| pancreatitis | inflammatory |
| cholocystitis | inflammatory |
| polycystic kidney disease | inflammatory |
| cancer | inflammatory |
| osteoarthritis | inflammatory |
| rheumatoid arthritis | inflammatory |
| spondyloarthritis | inflammatory |
| systemic juvenile idiopathic arthritis | inflammatory |
| psoriatic arthritis | inflammatory |
| gout | inflammatory |
| ankylosing spondylitis | inflammatory |
| juvenile rheumatoid arthritis | inflammatory |
| obesity | metabolic |
| cancer | metabolic |
| heart disease | metabolic |
| diabetes | metabolic |
| Cushing's disease | metabolic |
| polycystic ovary syndrome | metabolic |
| hypertension | metabolic |
| dyslipidemia | metabolic |
| stroke | metabolic |
| gallbladder disease | metabolic |
| osteoarthritis | metabolic |
| sleep apnea | metabolic |
| breathing problems | metabolic |
| depression | metabolic |
| anxiety | metabolic |
| pain | metabolic |
| lysosomal storage diseases (LSDs) | metabolic |
| congenital disorders of glycosylation | metabolic |
| metabolic disorder | metabolic |
| Activator Deficiency | metabolic |
| Alpha-mannosidosis | metabolic |
| Aspartylglucosaminuria | metabolic |
| Cholesteryl ester storage disease | metabolic |
| Chronic Hexosaminidase A Deficiency | metabolic |
| Cystinosis | metabolic |
| Danon disease | metabolic |
| Gaucher disease | metabolic |
| Fabry disease | metabolic |
| Farber disease | metabolic |
| Fucosidosis | metabolic |
| Galactosialidosis | metabolic |
| GM1 gangliosidosis | metabolic |
| I-Cell disease | metabolic |
| Infantile Free Sialic Acid Storage Disease | metabolic |
| Krabbe disease | metabolic |
| Metachromatic Leukodystrophy | metabolic |
| Pompe disease | metabolic |
| Mucopolysaccharidosis I | metabolic |
| Hurler syndrome | metabolic |
| Hurler-Scheie syndrome | metabolic |
| Scheie syndrome | metabolic |
| Mucopolysaccharidosis II | metabolic |
| Hunter syndrome | metabolic |
| Mucopolysaccharidosis IV | metabolic |
| Mucopolysaccharidosis VI | metabolic |
| Lysosomal Acid lipase deficiency | metabolic |
| Thrombocytopenia | metabolic |
| Maroteaux-Lamy syndrome | metabolic |
| Sly syndrome | metabolic |
| Pycnodysostosis | metabolic |
| Sandhoff disease | metabolic |
| Schindler disease | metabolic |
| Salla disease | metabolic |
| Tay-Sachs | metabolic |
| Wolman disease | metabolic |
| infection | ocular |
| refractive error | ocular |
| age related macular degeneration | ocular |
| cystoid macular edema | ocular |
| cataract | ocular |
| diabetic retinopathy | ocular |
| non-proliferative diabetic retinopathy | ocular |
| proliferative diabetic retinopathy | ocular |
| non-proliferative diabetic macular edema | ocular |
| proliferative diabetic macular edema | ocular |
| glaucoma | ocular |
| amblyopia | ocular |
| strabismus | ocular |
| color blindness | ocular |
| cytomegalovirus retinitis | ocular |
| keratoconus | ocular |
| diabetic macular edema | ocular |
| low vision | ocular |
| ocular hypertension | ocular |
| retinal detachment | ocular |
| eyelid twitching | ocular |
| inflammation | ocular |
| uveitis | ocular |
| bulging eye | ocular |
| dry eye disease | ocular |
| floater | ocular |
| xerophthalmia | ocular |
| diplopia | ocular |
| Graves' disease | ocular |
| night blindness | ocular |
| eye strain | ocular |
| red eye | ocular |
| nystagmus | ocular |
| presbyopia | ocular |
| excess tearing | ocular |
| retinal disorder | ocular |
| conjunctivitis | ocular |
| cancer | ocular |
| corneal ulcer | ocular |
| corneal abrasion | ocular |
| snow blindness | ocular |
| scleritis | ocular |
| keratitis | ocular |
| Thygeson's superficial punctate keratopathy | ocular |
| corneal neovascularization | ocular |
| Fuch's dystrophy | ocular |
| keratoconjuctitivis sicca | ocular |
| iritis | ocular |
| chorioretinal inflammation | ocular |
| chorioretinitis | ocular |
| choroiditis | ocular |
| retinitis | ocular |
| retinochoroiditis | ocular |
| pars planitis | ocular |
| Harada's disease | ocular |
| aniridia | ocular |
| macular scar | ocular |
| solar retinopathy | ocular |
| choroidal degeneration | ocular |
| choroidal dystrophy | ocular |
| choroideremia | ocular |
| gyrate atrophy | ocular |
| choroidal hemorrhage | ocular |
| choroidal detachment | ocular |
| retinoschisis | ocular |
| hypertensive retinopathy | ocular |
| Bull's eye maculopathy | ocular |
| epiretinal membrane | ocular |
| peripheral retinal degeneration | ocular |
| hereditary retinal dystrophy | ocular |
| retinitis pigmentosa | ocular |
| retinal hemorrhage | ocular |
| separation of retinal layers | ocular |
| retinal vein occlusion | ocular |
| visual impairment | ocular |
| refractory glaucoma | ocular |
| neovascular glaucoma | ocular |
| uveitic glaucoma | ocular |
| Pain | pain |
| nociceptive pain | pain |
| neuropathic pain | pain |
| psychogenic pain | pain |
| breakthrough pain | pain |
| incident pain | pain |
| back pain | pain |
| musculoskeletal pain | pain |
| post-operative pain | pain |
| operative pain | pain |
| visceral pain | pain |
| joint pain | pain |
| acute pain | pain |
| inflammatory pain | pain |
| knee pain | pain |
| dental pain | pain |
| chronic pain | pain |
| parasitic worm | infectious - parasite |
| cestode | infectious - parasite |
| nematode | infectious - parasite |
| trematode | infectious - parasite |
| Acanthocephala | infectious - parasite |
| Ascariasis | infectious - parasite |
| roundworms | infectious - parasite |
| Cestoda | infectious - parasite |
| tapeworm | infectious - parasite |
| Taenia saginata | infectious - parasite |
| human beef tapeworm | infectious - parasite |
| Taenia solium | infectious - parasite |
| human pork tapeworm | infectious - parasite |
| Diphyllobothrium latum | infectious - parasite |
| fish tapeworm | infectious - parasite |
| Echinococcosis | infectious - parasite |
| hydatid tapeworm | infectious - parasite |
| Clonorchis sinensis | infectious - parasite |
| Chinese liver fluke | infectious - parasite |
| Dracunculus medinensis | infectious - parasite |
| Guinea worm | infectious - parasite |
| Enterobius vermicularis | infectious - parasite |
| pinworm | infectious - parasite |
| Filariasis | infectious - parasite |
| Hookworm | infectious - parasite |
| Loa loa | infectious - parasite |
| Onchocerciasis | infectious - parasite |
| Schistosomiasis | infectious - parasite |
| Strongyloides stercoralis | infectious - parasite |
| Tapeworm | infectious - parasite |
| Toxocara canis | infectious - parasite |
| dog roundworm | infectious - parasite |
| Trichinella | infectious - parasite |
| Whipworm | infectious - parasite |
| Protozoan | infectious - parasite |
| Entamoeba histolytica | infectious - parasite |
| Entamoeba coli, Acanthamoeba | infectious - parasite |
| Balamuthia mandrillaris | infectious - parasite |
| Giardia | infectious - parasite |
| Cyclospora cayetanensis | infectious - parasite |
| Cryptosporidium | infectious - parasite |
| Toxoplasma gondii | infectious - parasite |
| Leishmania | infectious - parasite |
| L. tropica | infectious - parasite |
| L. donovani | infectious - parasite |
| L. Mexicana | infectious - parasite |
| Plasmodium | infectious - parasite |
| Malaria | infectious - parasite |
| P. falciparum | infectious - parasite |
| P. vivax | infectious - parasite |
| P. malariae | infectious - parasite |
| Babesia | infectious - parasite |
| athropoda | infectious - parasite |
| Acari Varroa destructor | infectious - parasite |
| Cymothoa exigua | infectious - parasite |
| Bed bug | infectious - parasite |
| Culicidae | infectious - parasite |
| mosquitoes | infectious - parasite |
| Calyptra | infectious - parasite |
| vampire moth | infectious - parasite |
| Hippoboscoidea Tsetse fly | infectious - parasite |
| Lipoptena | infectious - parasite |
| Melophagus ovinus | infectious - parasite |
| sheep ked | infectious - parasite |
| Oestridae | infectious - parasite |
| bot flies | infectious - parasite |
| Human botfly | infectious - parasite |
| Phlebotominae | infectious - parasite |
| sand flies | infectious - parasite |
| Phthiraptera | infectious - parasite |
| Lice | infectious - parasite |
| Body louse | infectious - parasite |
| Crab louse | infectious - parasite |
| Head louse | infectious - parasite |
| Siphonaptera | infectious - parasite |
| fleas | infectious - parasite |
| Tabanidae | infectious - parasite |
| horse flies | infectious - parasite |
| Tantulocarida | infectious - parasite |
| Triatominae | infectious - parasite |
| Pea crab | infectious - parasite |
| Sacculina | infectious - parasite |
| annelid | infectious - parasite |
| hirudinea | infectious - parasite |
| monogenean | infectious - parasite |
| flatworm | infectious - parasite |
| Calydiscoides euzeti | infectious - parasite |
| Lethacotyle vera | infectious - parasite |
| Protocotyle euzetmaillardi | infectious - parasite |
| Pseudorhabdosynochus | infectious - parasite |
| mollusk | infectious - parasite |
| cancellaria cooperii | infectious - parasite |
| Glochidium | infectious - parasite |
| Pyramidellidae | infectious - parasite |
| chordate | infectious - parasite |
| Cookiecutter shark | infectious - parasite |
| Candiru | infectious - parasite |
| Lampreys | infectious - parasite |
| Male Deep sea anglers | infectious - parasite |
| False cleanerfisb | infectious - parasite |
| Hood mockingbird | infectious - parasite |
| Oxpeckers | infectious - parasite |
| Snubnosed eel | infectious - parasite |
| Vampire bat | infectious - parasite |
| Vampire finch | infectious - parasite |
| Cuckoo | infectious - parasite |
| Cowbird | infectious - parasite |
| brood parasite | infectious - parasite |
| parasite | infectious - parasite |
| Toxoplasmosis | infectious - parasitic disease |
| Acanthamoeba keratitis | infectious - parasitic disease |
| Leishmaniasis | infectious - parasitic disease |
| Babesiosis | infectious - parasitic disease |
| Granulomatous amoebic encephalitis | infectious - parasitic disease |
| Cryptosporidiosis | infectious - parasitic disease |
| Cyclosporiasis | infectious - parasitic disease |
| Primary amoebic meningoencephalitis | infectious - parasitic disease |
| Ascariasis | infectious - parasitic disease |
| Enterobiasis | infectious - parasitic disease |
| Strongyloidiasis | infectious - parasitic disease |
| Toxocariasis | infectious - parasitic disease |
| dracunculiasis | infectious - parasitic disease |
| Hookworm | infectious - parasitic disease |
| Tapeworm | infectious - parasitic disease |
| Whipworm | infectious - parasitic disease |
| scabies | infectious - parasitic disease |
| headlice | infectious - parasitic disease |
| pubic lice | infectious - parasitic disease |
| myiasis | infectious - parasitic disease |
| tungiasis | infectious - parasitic disease |
| Ticks (Ixodoidea) | infectious - parasitic disease |
| schistosomiasis | infectious - parasitic disease |
| gnathostomiasis | infectious - parasitic disease |
| paragonimiasis | infectious - parasitic disease |
| fascioliasis | infectious - parasitic disease |
| swimmer's itch | infectious - parasitic disease |
| Granulomatous amoebic encephalitis | infectious - parasitic disease |
| Acanthamoeba keratitis | infectious - parasitic disease |
| Babesiosis | infectious - parasitic disease |
| Balantidiasis | infectious - parasitic disease |
| Blastocystosis | infectious - parasitic disease |
| Cryptosporidiosis | infectious - parasitic disease |
| Cyclosporiasis | infectious - parasitic disease |
| Dientamoebiasis | infectious - parasitic disease |
| Amoebiasis | infectious - parasitic disease |
| Giardiasis | infectious - parasitic disease |
| Isosporiasis | infectious - parasitic disease |
| Leishmaniasis | infectious - parasitic disease |
| Primary amoebic meningoencephalitis | infectious - parasitic disease |
| Malaria | infectious - parasitic disease |
| Rhinosporidiosis | infectious - parasitic disease |
| Sarcocystosis | infectious - parasitic disease |
| Toxoplasmosis (Acute and Latent) | infectious - parasitic disease |
| Trichomoniasis | infectious - parasitic disease |
| Sleeping sickness | infectious - parasitic disease |
| Chagas disease | infectious - parasitic disease |
| Tapeworm | infectious - parasitic disease |
| Diphyllobothriasis | infectious - parasitic disease |
| Echinococcosis | infectious - parasitic disease |
| Hymenolepiasis | infectious - parasitic disease |
| Beef tapeworm | infectious - parasitic disease |
| Cysticercosis | infectious - parasitic disease |
| Bertielliasis | infectious - parasitic disease |
| Sparganosis | infectious - parasitic disease |
| Clonorchiasis | infectious - parasitic disease |
| Lancet liver fluke | infectious - parasitic disease |
| Liver fluke | infectious - parasitic disease |
| Fasciolopsiasis | infectious - parasitic disease |
| Metagonimiasis | infectious - parasitic disease |
| Metorchiasis | infectious - parasitic disease |
| Chinese liver fluke | infectious - parasitic disease |
| Paragonimiasis, lung fluke | infectious - parasitic disease |
| Schistosomiasis | infectious - parasitic disease |
| intestinal schistosomiasis | infectious - parasitic disease |
| urinary schistosomiasis | infectious - parasitic disease |
| Schistosomiasis | infectious - parasitic disease |
| Asian intestinal schistosomiasis | infectious - parasitic disease |
| Fasciolosis | infectious - parasitic disease |
| Swimmer's itch | infectious - parasitic disease |
| Intestinal fluke | infectious - parasitic disease |
| bilharzia | infectious - parasitic disease |
| bilharziosis | infectious - parasitic disease |
| snail fever | infectious - parasitic disease |
| Ancylostomiasis | infectious - parasitic disease |
| Angiostrongyliasis | infectious - parasitic disease |
| Anisakiasis | infectious - parasitic disease |
| Roundworm | infectious - parasitic disease |
| Baylisascariasis | infectious - parasitic disease |
| lymphatic filariasis | infectious - parasitic disease |
| Dioctophyme renalis infection | infectious - parasitic disease |
| Dracunculiasis | infectious - parasitic disease |
| Enterobiasis | infectious - parasitic disease |
| Gnathostomiasis | infectious - parasitic disease |
| Halicephalobiasis | infectious - parasitic disease |
| Loa filariasis | infectious - parasitic disease |
| Mansonelliasis | infectious - parasitic disease |
| River blindness | infectious - parasitic disease |
| Strongyloidiasis | infectious - parasitic disease |
| Thelaziasis | infectious - parasitic disease |
| Toxocariasis | infectious - parasitic disease |
| Trichinosis | infectious - parasitic disease |
| Whipworm | infectious - parasitic disease |
| Elephantiasis | infectious - parasitic disease |
| Hookworm | infectious - parasitic disease |
| Parasitic pneumonia | infectious - parasitic disease |
| Calabar swellings | infectious - parasitic disease |
| filariasis | infectious - parasitic disease |
| onchocerciasis | infectious - parasitic disease |
| Parasitic pneumonia | infectious - parasitic disease |
| Lymphatic filariasis | infectious - parasitic disease |
| Acanthocephaliasis | infectious - parasitic disease |
| Halzoun syndrome | infectious - parasitic disease |
| Myiasis | infectious - parasitic disease |
| Screwworm | infectious - parasitic disease |
| Chigoe flea | infectious - parasitic disease |
| Human botfly | infectious - parasitic disease |
| Cochliomyia | infectious - parasitic disease |
| Head louse | infectious - parasitic disease |
| Body louse | infectious - parasitic disease |
| Crab louse | infectious - parasitic disease |
| Demodex | infectious - parasitic disease |
| Scabies | infectious - parasitic disease |
| “Chiggers” (Trombiculidae) | infectious - parasitic disease |
| Flea, Siphonaptera | infectious - parasitic disease |
| Bedbug | infectious - parasitic disease |
| Tick | infectious - parasitic disease |
| Pediculosis | infectious - parasitic disease |
| Trombiculosis | infectious - parasitic disease |
| psychological disorder | psychological |
| Aboulia | psychological |
| Absence epilepsy | psychological |
| Acute stress Disorder | psychological |
| Adjustment Disorders | psychological |
| Adverse effects of medication NOS | psychological |
| Age related cognitive decline | psychological |
| Agoraphobia | psychological |
| Alcohol Addiction | psychological |
| Alzheimer's Disease | psychological |
| Amnesia (also known as Amnestic Disorder) | psychological |
| Amphetamine Addiction | psychological |
| Anorexia Nervosa | psychological |
| Anterograde amnesia | psychological |
| Antisocial personality disorder (also known as | psychological |
| Sociopathy) | |
| Anxiety Disorder (Also known as Generalized Anxiety | psychological |
| Disorder) | |
| Anxiolytic related disorders | psychological |
| Asperger's Syndrome (now part of Autism Spectrum | psychological |
| Disorder) | |
| Attention Deficit Disorder (Also known as ADD) | psychological |
| Attention Deficit Hyperactivity Disorder (Also known as | psychological |
| ADHD) | |
| Autism Spectrum Disorder (also known as Autism) | psychological |
| Autophagia | psychological |
| Avoidant Personality Disorder | psychological |
| Barbiturate related disorders | psychological |
| Benzodiazepine related disorders | psychological |
| Bereavement | psychological |
| Bibliomania | psychological |
| Binge Eating Disorder | psychological |
| Bipolar disorder (also known as Manic Depression) | psychological |
| Bipolar I | psychological |
| Bipolar II | psychological |
| Body Dysmorphic Disorder | psychological |
| Borderline intellectual functioning | psychological |
| Borderline Personality Disorder | psychological |
| Breathing-Related Sleep Disorder | psychological |
| Brief Psychotic Disorder | psychological |
| Bruxism | psychological |
| Bulimia Nervosa | psychological |
| Caffeine Addiction | psychological |
| Cannabis Addiction | psychological |
| Catatonic disorder | psychological |
| Catatonic schizophrenia | psychological |
| Childhood amnesia | psychological |
| Childhood Disintegrative Disorder (now part of Autism | psychological |
| Spectrum Disorder) | |
| Childhood Onset Fluency Disorder (formerly known as | psychological |
| Stuttering) | |
| Circadian Rhythm Disorders | psychological |
| Claustrophobia | psychological |
| Cocaine related disorders | psychological |
| Communication disorder | psychological |
| Conduct Disorder | psychological |
| Conversion Disorder | psychological |
| Cotard delusion | psychological |
| Cyclothymia (also known as Cyclothymic Disorder) | psychological |
| Delerium | psychological |
| Delusional Disorder | psychological |
| dementia | psychological |
| Dependent Personality Disorder (also known as Asthenic | psychological |
| Personality Disorder) | |
| Depersonalization disorder (now known as | psychological |
| Depersonalization/Derealization Disorder) | |
| Depression (also known as Major Depressive Disorder) | psychological |
| Depressive personality disorder | psychological |
| Derealization disorder (now known as Depersonalization/ | psychological |
| Derealization Disorder) | |
| Dermotillomania | psychological |
| Desynchronosis | psychological |
| Developmental coordination disorder | psychological |
| Diogenes Syndrome | psychological |
| Disorder of written expression | psychological |
| Dispareunia | psychological |
| Dissocial Personality Disorder | psychological |
| Dissociative Amnesia | psychological |
| Dissociative Fugue | psychological |
| Dissociative Identity Disorder (formerly known as | psychological |
| Multiple Personality Disorder) | |
| Down syndrome | psychological |
| Dyslexia | psychological |
| Dyspareunia | psychological |
| Dysthymia (now known as Persistent Depressive | psychological |
| Disorder) | |
| Eating disorder NOS | psychological |
| Ekbom's Syndrome (Delusional Parasitosis) | psychological |
| Emotionally unstable personality disorder | psychological |
| Encopresis | psychological |
| Enuresis (bedwetting) | psychological |
| Erotomania | psychological |
| Exhibitionistic Disorder | psychological |
| Expressive language disorder | psychological |
| Factitious Disorder | psychological |
| Female Sexual Disorders | psychological |
| Fetishistic Disorder | psychological |
| Folie à deux | psychological |
| Fregoli delusion | psychological |
| Frotteuristic Disorder | psychological |
| Fugue State | psychological |
| Ganser syndrome | psychological |
| Gambling Addiction | psychological |
| Gender Dysphoria (formerly known as Gender Identity | psychological |
| Disorder) | |
| Generalized Anxiety Disorder | psychological |
| General adaptation syndrome | psychological |
| Grandiose delusions | psychological |
| Hallucinogen Addiction | psychological |
| Haltlose personality disorder | psychological |
| Histrionic Personality Disorder | psychological |
| Primary hypersomnia | psychological |
| Huntington's Disease | psychological |
| Hypoactive sexual desire disorder | psychological |
| Hypochondriasis | psychological |
| Hypomania | psychological |
| Hyperkinetic syndrome | psychological |
| Hypersomnia | psychological |
| Hysteria | psychological |
| Impulse control disorder | psychological |
| Impulse control disorder NOS | psychological |
| Inhalant Addiction | psychological |
| Insomnia | psychological |
| Intellectual Development Disorder | psychological |
| Intermittent Explosive Disorder | psychological |
| Joubert syndrome | psychological |
| Kleptomania | psychological |
| Korsakoff's syndrome | psychological |
| Lacunar amnesia | psychological |
| Language Disorder | psychological |
| Learning Disorders | psychological |
| Major Depression (also known as Major Depressive | psychological |
| Disorder) | |
| major depressive disorder | psychological |
| Male Sexual Disorders | psychological |
| Malingering | psychological |
| Mathematics disorder | psychological |
| Medication-related disorder | psychological |
| Melancholia | psychological |
| Mental Retardation (now known as Intellectual | psychological |
| Development Disorder) | |
| Misophonia | psychological |
| Morbid jealousy | psychological |
| Multiple Personality Disorder (now known as | psychological |
| Dissociative Identity Disorder) | |
| Munchausen Syndrome | psychological |
| Munchausen by Proxy | psychological |
| Narcissistic Personality Disorder | psychological |
| Narcolepsy | psychological |
| Neglect of child | psychological |
| Neurocognitive Disorder (formerly known as Dementia) | psychological |
| Neuroleptic-related disorder | psychological |
| Nightmare Disorder | psychological |
| Non Rapid Eye Movement | psychological |
| Obsessive-Compulsive Disorder | psychological |
| Obsessive-Compulsive Personality Disorder (also known | psychological |
| as Anankastic Personality Disorder) | |
| Oneirophrenia | psychological |
| Onychophagia | psychological |
| Opioid Addiction | psychological |
| Oppositional Defiant Disorder | psychological |
| Orthorexia (ON) | psychological |
| Pain disorder | psychological |
| Panic attacks | psychological |
| Panic Disorder | psychological |
| Paranoid Personality Disorder | psychological |
| Parkinson's Disease | psychological |
| Partner relational problem | psychological |
| Passive-aggressive personality disorder | psychological |
| Pathological gambling | psychological |
| Pedophilic Disorder | psychological |
| Perfectionism | psychological |
| Persecutory delusion | psychological |
| Persistent Depressive Disorder (also known as | psychological |
| Dysthymia) | |
| Personality change due to a general medical | psychological |
| condition | |
| Personality disorder | psychological |
| Pervasive developmental disorder (PDD) | psychological |
| Phencyclidine related disorder | psychological |
| Phobic disorder | psychological |
| Phonological disorder | psychological |
| Physical abuse | psychological |
| Pica | psychological |
| Polysubstance related disorder | psychological |
| Postpartum Depression | psychological |
| Post-traumatic embitterment disorder (PTED) | psychological |
| Post-Traumatic Stress Disorder | psychological |
| Premature ejaculation | psychological |
| Premenstrual Dysphoric Disorder | psychological |
| Psychogenic amnesia | psychological |
| Psychological factor affecting medical condition | psychological |
| Psychoneurotic personality disorder | psychological |
| Psychotic disorder | psychological |
| Pyromania | psychological |
| Reactive Attachment Disorder | psychological |
| Reading disorder | psychological |
| Recurrent brief depression | psychological |
| Relational disorder | psychological |
| REM Sleep Behavior Disorder | psychological |
| Restless Leg Syndrome | psychological |
| Retrograde amnesia | psychological |
| Retts Disorder (now part of Autism Spectrum Disorder) | psychological |
| Rumination syndrome | psychological |
| Sadistic personality disorder | psychological |
| Schizoaffective Disorder | psychological |
| Schizoid Personality Disorder | psychological |
| Schizophrenia | psychological |
| Schizophreniform disorder | psychological |
| Schizotypal Personality Disorder | psychological |
| Seasonal Affective Disorder | psychological |
| Sedative | psychological |
| Hypnotic | psychological |
| or Anxiolytic Addiction | psychological |
| Selective Mutism | psychological |
| Self-defeating personality disorder | psychological |
| Separation Anxiety Disorder | psychological |
| Sexual Disorders Female | psychological |
| Sexual Disorders Male | psychological |
| Sexual Addiction | psychological |
| Sexual Masochism Disorder | psychological |
| Sexual Sadism Disorder | psychological |
| Shared Psychotic Disorder | psychological |
| Sleep Arousal Disorders | psychological |
| Sleep Paralysis | psychological |
| Sleep Terror Disorder | psychological |
| Nightmare Disorder | psychological |
| Social Anxiety Disorder | psychological |
| Somatization Disorder | psychological |
| Specific Phobias | psychological |
| Stendhal syndrome | psychological |
| Stereotypic movement disorder | psychological |
| Stimulant Addiction | psychological |
| Stuttering (now known as Childhood Onset Fluency | psychological |
| Disorder) | |
| Substance related disorder | psychological |
| Tardive dyskinesia | psychological |
| Tobacco Addiction | psychological |
| Tourettes Syndrome | psychological |
| Transient tic disorder | psychological |
| Transient global amnesia | psychological |
| Transvestic Disorder | psychological |
| Trichotillomania | psychological |
| Undifferentiated Somatoform Disorder | psychological |
| Vaginismus | psychological |
| Voyeuristic Disorder | psychological |
| rare disease | rare disease |
| Acrocephalosyndactylia | rare disease |
| Acrodermatitis | rare disease |
| Addison Disease | rare disease |
| Adie Syndrome | rare disease |
| Alagille Syndrome | rare disease |
| Amylose | rare disease |
| Amyotrophic Lateral Sclerosis | rare disease |
| Angelman Syndrome | rare disease |
| Angiolymphoid Hyperplasia with Eosinophilia | rare disease |
| Arnold-Chiari Malformation | rare disease |
| Arthritis | rare disease |
| Juvenile Rheumatoid | rare disease |
| Asperger Syndrome | rare disease |
| Bardet-Biedl Syndrome | rare disease |
| Barnett Esophagus | rare disease |
| Beckwith-Wiedemann Syndrome | rare disease |
| Behcet Syndrome | rare disease |
| Bloom Syndrome | rare disease |
| Bowen's Disease | rare disease |
| Brachial Plexus Neuropathies | rare disease |
| Brown-Sequard Syndrome | rare disease |
| Budd-Chiari Syndrome | rare disease |
| Burkitt Lymphoma | rare disease |
| Carcinoma 256 | rare disease |
| Walker | rare disease |
| Caroli Disease | rare disease |
| Charcot-Marie-Tooth Disease | rare disease |
| Chediak-Higashi Syndrome | rare disease |
| Chiari-Frommel Syndrome | rare disease |
| Chondrodysplasia Punctata | rare disease |
| Colonic Pseudo-Obstruction | rare disease |
| Colorectal Neoplasms | rare disease |
| Hereditary Nonpolyposis | rare disease |
| Craniofacial Dysostosis | rare disease |
| Creutzfeldt-Jakob Syndrome | rare disease |
| Crohn Disease | rare disease |
| Cushing Syndrome | rare disease |
| Cystic Fibrosis | rare disease |
| Dandy-Walker Syndrome | rare disease |
| De Lange Syndrome | rare disease |
| Dementia | rare disease |
| Vascular | rare disease |
| Dermatitis Herpetiformis | rare disease |
| DiGeorge Syndrome | rare disease |
| Diffuse Cerebral Sclerosis of Schilder | rare disease |
| Duane Retraction Syndrome | rare disease |
| Dupuytren Contracture | rare disease |
| Ebstein Anomaly | rare disease |
| Eisenmenger Complex | rare disease |
| Ellis-Van Creveld Syndrome | rare disease |
| Encephalitis | rare disease |
| Enchondromatosis | rare disease |
| Epidermal Necrolysis | rare disease |
| Toxic | rare disease |
| Facial Hemiatrophy | rare disease |
| Factor XII Deficiency | rare disease |
| Fanconi Anemia | rare disease |
| Felty's Syndrome | rare disease |
| Fibrous Dysplasia | rare disease |
| Polyostotic | rare disease |
| Fox-Fordyce Disease | rare disease |
| Friedreich Ataxia | rare disease |
| Fusobacterium | rare disease |
| Gardner Syndrome | rare disease |
| Gaucher Disease | rare disease |
| Gerstmann Syndrome | rare disease |
| Giant Lymph Node Hyperplasia | rare disease |
| Glycogen Storage Disease Type I | rare disease |
| Glycogen Storage Disease Type II | rare disease |
| Glycogen Storage Disease Type IV | rare disease |
| Glycogen Storage Disease Type V | rare disease |
| Glycogen Storage Disease Type VII | rare disease |
| Goldenhar Syndrome | rare disease |
| Guillain-Barre Syndrome | rare disease |
| Hallermann's Syndrome | rare disease |
| Hamartoma Syndrome | rare disease |
| Multiple | rare disease |
| Hartnup Disease | rare disease |
| Hepatolenticular Degeneration | rare disease |
| Hepatolenticular Degeneration | rare disease |
| Hereditary Sensory and Motor Neuropathy | rare disease |
| Hirschsprung Disease | rare disease |
| Histiocytic Necrotizing Lymphadenitis | rare disease |
| Histiocytosis | rare disease |
| Langerhans-Cell | rare disease |
| Hodgkin Disease | rare disease |
| Horner Syndrome | rare disease |
| Huntington Disease | rare disease |
| Hyperaldosteronism | rare disease |
| Hyperhidrosis | rare disease |
| Hyperostosis | rare disease |
| Diffuse Idiopathic Skeletal | rare disease |
| Hypopituitarism | rare disease |
| Inappropriate ADH Syndrome | rare disease |
| Intestinal Polyps | rare disease |
| Isaacs Syndrome | rare disease |
| Kartagener Syndrome | rare disease |
| Kearns-Sayre Syndrome | rare disease |
| Klippel-Feil Syndrome | rare disease |
| Klippel-Trenaunay-Weber Syndrome | rare disease |
| Kluver-Bucy Syndrome | rare disease |
| Korsakoff Syndrome | rare disease |
| Lafora Disease | rare disease |
| Lambert-Eaton Myasthenic Syndrome | rare disease |
| Landau-Kleffner Syndrome | rare disease |
| Langer-Giedion Syndrome | rare disease |
| Leigh Disease | rare disease |
| Lesch-Nyhan Syndrome | rare disease |
| Leukodystrophy | rare disease |
| Globoid Cell | rare disease |
| Li-Fraumeni Syndrome | rare disease |
| Long QT Syndrome | rare disease |
| Machado-Joseph Disease | rare disease |
| Mallory-Weiss Syndrome | rare disease |
| Marek Disease | rare disease |
| Marfan Syndrome | rare disease |
| Meckel Diverticulum | rare disease |
| Meige Syndrome | rare disease |
| Melkersson-Rosenthal Syndrome | rare disease |
| Meniere Disease | rare disease |
| Mikulicz’ Disease | rare disease |
| Miller Fisher Syndrome | rare disease |
| Mobius Syndrome | rare disease |
| Moyamoya Disease | rare disease |
| Mucocutaneous Lymph Node Syndrome | rare disease |
| Mucopolysaccharidosis I | rare disease |
| Mucopolysaccharidosis II | rare disease |
| Mucopolysaccharidosis III | rare disease |
| Mucopolysaccharidosis IV | rare disease |
| Mucopolysaccharidosis VI | rare disease |
| Multiple Endocrine Neoplasia Type 1 | rare disease |
| Munchausen Syndrome by Proxy | rare disease |
| Muscular Atrophy | rare disease |
| Spinal | rare disease |
| Narcolepsy | rare disease |
| Neuroaxonal Dystrophies | rare disease |
| Neuromyelitis Optica | rare disease |
| Neuronal Ceroid-Lipofuscinoses | rare disease |
| Niemann-Pick Diseases | rare disease |
| Noonan Syndrome | rare disease |
| Optic Atrophies | rare disease |
| Hereditary | rare disease |
| Osteitis Deformans | rare disease |
| Osteochondritis | rare disease |
| Osteochondrodysplasias | rare disease |
| Osteolysis | rare disease |
| Osteoarthritis | rare disease |
| Essential | rare disease |
| Paget Disease Extramammary | rare disease |
| Paget's Disease | rare disease |
| Mammary | rare disease |
| Panniculitis | rare disease |
| Nodular Nonsuppurative | rare disease |
| Papillon-Lefevre Disease | rare disease |
| Paralysis | rare disease |
| Pelizaeus-Merzbacher Disease | rare disease |
| Pemphigus | rare disease |
| Benign Familial | rare disease |
| Penile Induration | rare disease |
| Pericarditis | rare disease |
| Constrictive | rare disease |
| Peroxisomal Disorders | rare disease |
| Peutz-Jeghers Syndrome | rare disease |
| Pick Disease of the Brain | rare disease |
| Pierre Robin Syndrome | rare disease |
| Pigmentation Disorders | rare disease |
| Pityriasis Lichenoides | rare disease |
| Polycystic Ovary Syndrome | rare disease |
| Polyendocrinopathies | rare disease |
| Autoimmune | rare disease |
| Prader-Willi Syndrome | rare disease |
| Pupil Disorders | rare disease |
| Rett Syndrome | rare disease |
| Reye Syndrome | rare disease |
| Rubinstein-Taybi Syndrome | rare disease |
| Sandhoff Disease | rare disease |
| Sarcoma | rare disease |
| Ewing's | rare disease |
| Schnitzler Syndrome | rare disease |
| Sjogren's Syndrome | rare disease |
| Sjogren-Larsson Syndrome | rare disease |
| Smith-Lemli-Opitz Syndrome | rare disease |
| Spinal Muscular Atrophies of Childhood | rare disease |
| Sturge-Weber Syndrome | rare disease |
| Sweating | rare disease |
| Gustatory | rare disease |
| Takayasu Arteritis | rare disease |
| Tangier Disease | rare disease |
| Tay-Sachs Disease | rare disease |
| Thromboangiitis Obliterans | rare disease |
| Thyroiditis | rare disease |
| Autoimmune | rare disease |
| Tietze's Syndrome | rare disease |
| Togaviridae Infections | rare disease |
| Tolosa-Hunt Syndrome | rare disease |
| Tourette Syndrome | rare disease |
| Uveomeningoencephalitic Syndrome | rare disease |
| Waarderiburg's Syndrome | rare disease |
| Wegener Granulomatosis | rare disease |
| Weil Disease | rare disease |
| Werner Syndrome | rare disease |
| Williams Syndrome | rare disease |
| Wilms Tumor | rare disease |
| Wolff-Parkinson-White Syndrome | rare disease |
| Wolfram Syndrome | rare disease |
| Wolman Disease | rare disease |
| Zellweger Syndrome | rare disease |
| Zollinger-Ellison Syndrome | rare disease |
| von Willebrand Disease | rare disease |
| animal disease | veterinary |
| Acute hepatopancreatic necrosis disease | veterinary |
| Aflatoxicosis | veterinary |
| African swine fever | veterinary |
| Akabane | veterinary |
| Anthrax | veterinary |
| Australian bat lyssavirus | veterinary |
| Avian influenza (bird flu) | veterinary |
| Avian paramyxovirus | veterinary |
| Blue-green algae (cyanobacteria) | veterinary |
| Bluetongue | veterinary |
| Botulism | veterinary |
| Botulism in poultry | veterinary |
| Bovine ephemeral fever | veterinary |
| Bovine tuberculosis | veterinary |
| Bovine virus diarrhea | veterinary |
| Brucellosis | veterinary |
| Brucella ovis | veterinary |
| Buffalo fly | veterinary |
| Campylobacteriosis | veterinary |
| vibriosis | veterinary |
| Caprine arthritis encephalitis (CAE) | veterinary |
| Cat-scratch disease | veterinary |
| Cattle ticks | veterinary |
| Classical swine fever | veterinary |
| Clostridial diseases | veterinary |
| Copper deficiency | veterinary |
| Cryptococcosis | veterinary |
| Dry eye disease | veterinary |
| Enzootic bovine leucosis (EBL) | veterinary |
| Epizootic ulcerative syndrome (red-spot disease) | veterinary |
| Equine herpesvirus | veterinary |
| Equine infectious anaemia (EIA) | veterinary |
| Equine influenza | veterinary |
| Equine viral arteritis (EVA) | veterinary |
| Foot and mouth disease | veterinary |
| Fowl cholera | veterinary |
| Fowl pox | veterinary |
| Giardiasis | veterinary |
| Hendra virus | veterinary |
| Hydatid disease (hydatid cysts) | veterinary |
| Infectious laryngotracheitis | veterinary |
| Japanese encephalitis | veterinary |
| Johne's disease | veterinary |
| Leptospirosis | veterinary |
| Listeriosis | veterinary |
| Lumpy jaw | veterinary |
| Marek's disease | veterinary |
| Melioidosis | veterinary |
| Neospora caninum | veterinary |
| Newcastle disease | veterinary |
| Nipah virus | veterinary |
| Nosema | veterinary |
| Ovine brucellosis | veterinary |
| Pestivirus | veterinary |
| Pimelea poisoning | veterinary |
| St George disease | veterinary |
| marree disease | veterinary |
| Psittacosis | veterinary |
| ornithosis | veterinary |
| Q fever | veterinary |
| Rabies | veterinary |
| Rinderpest | veterinary |
| Ringworm | veterinary |
| Salmonellosis | veterinary |
| Screw-worm fly | veterinary |
| Skin fluke infestation | veterinary |
| Sparganosis | veterinary |
| Spotty liver | veterinary |
| Strangles | veterinary |
| African Swine fever | veterinary |
| Classical Swine fever | veterinary |
| Swine influenza | veterinary |
| Swine vesicular disease | veterinary |
| Tetanus | veterinary |
| Tick fever | veterinary |
| Toxocariasis | veterinary |
| Toxoplasmosis | veterinary |
| Transit tetany | veterinary |
| Transmissible spongiform encephalopathies | veterinary |
| Tuberculosis (TB) | veterinary |
| Vesicular exanthema | veterinary |
| Vesicular stomatitis | veterinary |
| Warts | veterinary |
| White nose syndrome | veterinary |
| White spot disease | veterinary |
| Wooden tongue and lumpy jaw | veterinary |
| animal cancer | veterinary - cancer |
| Lymphoma | veterinary - cancer |
| leukemia | veterinary - cancer |
| carcinoma | veterinary - cancer |
| sarcoma | veterinary - cancer |
| anus cancer | veterinary - cancer |
| bladder cancer | veterinary - cancer |
| bile duct cancer | veterinary - cancer |
| bone cancer | veterinary - cancer |
| brain cancer | veterinary - cancer |
| breast cancer | veterinary - cancer |
| cervical cancer | veterinary - cancer |
| chest cancer | veterinary - cancer |
| colon/rectum cancer | veterinary - cancer |
| connective tissue cancer | veterinary - cancer |
| endometrial cancer | veterinary - cancer |
| esophageal cancer | veterinary - cancer |
| eye cancer | veterinary - cancer |
| gallbladder cancer | veterinary - cancer |
| head and neck cancer | veterinary - cancer |
| liver cancer | veterinary - cancer |
| kidney cancer | veterinary - cancer |
| larynx cancer | veterinary - cancer |
| lung cancer | veterinary - cancer |
| mouth cancer | veterinary - cancer |
| nose cancer | veterinary - cancer |
| ovarian cancer | veterinary - cancer |
| pancreatic cancer | veterinary - cancer |
| penile cancer | veterinary - cancer |
| prostate cancer | veterinary - cancer |
| skin cancer | veterinary - cancer |
| small intestine cancer | veterinary - cancer |
| stomach cancer | veterinary - cancer |
| spinal marrow cancer | veterinary - cancer |
| tailbone cancer | veterinary - cancer |
| testicular cancer | veterinary - cancer |
| throat cancer | veterinary - cancer |
| thyroid cancer | veterinary - cancer |
| uterine cancer | veterinary - cancer |

### Autoimmune Indications

In some embodiments, therapeutic indications include autoimmune indications. As used herein, the term “autoimmune indication” refers to any therapeutic indication involving irritation or destruction to a subject by components of the subject's own immune system. In some embodiments, the immune system components are antibodies that bind to subject proteins.

Treatment of autoimmune indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation.

In some embodiments, autoimmune indications may include, but are not limited to, Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type I diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA). In some embodiments, autoimmune indications may include, but are not limited to, any of those listed in Table 5, above.

### Cancer-Related Indications

In some embodiments, therapeutic indications include cancer-related indications. The term “cancer” refers to a collection of diseases characterized by dysfunctional cell growth and division, in some cases spreading between bodily regions. As used herein, the term “cancer-related indication” refers to any disease, disorder, or condition pertaining to cancer, cancer treatment, or pre-cancerous conditions. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation.

Cancer-related indications include pathological conditions characterized by malignant neoplastic growths, tumors, and/or hematological malignancies. In some embodiments, cancer-related indications include but are not limited to, all types of lymphomas/leukemias, carcinomas and sarcomas, including cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid, and uterus, Additional cancer-related indications include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma, sinonasal undifferentiated carcinoma, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma, Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer, Additional cancer-related indications may include, but are not limited to, any of those listed in Table 5, above.

### Cardiac Indications

In some embodiments, therapeutic indications include cardiac indications. As used herein, the term “cardiac indication” refers to any disease, disorder, or condition related to the heart. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. In some embodiments, SBPs include stents used to keep arteries open. In some embodiments, SBPs include angioplasty guidewires or are coated onto angioplasty guidewires used to navigate blood vessels during surgical interventions.

Non-limiting examples of cardiac indications may include, but are not limited to, any of those listed in Table 5, above.

### Central Nervous System Indications

In some embodiments, therapeutic indications include central nervous system (CNS) indications. As used herein, the term “CNS indication” refers to any therapeutic indications related to the brain and/or network of nerves and tissues that control bodily activities. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. In some embodiments, SBPs may be used to provide enzyme replacement therapy products to the CNS.

CNS indications may include, but are not limited to, lysosomal storage diseases (LSD), mental retardation, seizures, profound neurodegeneration, behavioral abnormalities, psycho-motor defects, Mucopolysaccharidosis type II (Hunter Syndrome, iduronate sulfatase deficiency), Mucopolysaccharidosis type VI (Maroteaux-Lamy Syndrome, arylsulfatase B deficiency), Mucopolysaccharidosis type III (Sanfilippo A), Mucopolysaccharidosis type IV (MPS IV), Pompe disease (acid maltase deficiency), Niemann-Pick B (NP-B) disease, metachromatic leukodystrophy (MLD, Arylsufatase A deficiency), Krabbe disease, Wolman disease, Sly syndrome, Alzheimer's disease (AD), Huntington's Disease (HD), and Parkinson's disease (PD). Additional CNS indications may include, but are not limited to, any of those listed in Table 5, above.

In some embodiments, SBPs may be used to deliver monoclonal antibodies against protein aggregates in the CNS and CSF. Such antibodies may be used to treat degenerative diseases like Alzheimer's disease (AD), Huntington's Disease (HD) and Parkinson's disease (PD). In some embodiments, SBPs may be used to deliver and/or regulate neurotrophic factors in the CNS.

### Dryness

In some embodiments, therapeutic indications include dryness. In this context, “dryness” refers to any disease, disorder, or condition characterized by reduced hydration. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation.

Dryness causes discomfort and pain in many parts of the body. Areas commonly afflicted with dryness include, but are not limited to the skin, eye, vagina, mouth, and nose. In some embodiments, SBPs described herein may be used as a lubricant to treat symptoms of dryness, non-limiting examples of which include, redness, pain, itching, swelling, flaking, scaling, pealing, and tightness. In some embodiments, SBPs include silk fibroin as a lubricant. In some embodiments, methods of using SBPs may include any of those presented in International Publication Number WO2017139684 or United States Publication Number US20140235554, the contents of each of which are herein incorporated by reference in their entirety. In some embodiments, the treatment of dryness involves the administration of an SBP. In some embodiments, the SBPs are administered topically. In some embodiments, the SBP is in any format (e.g. solution or hydrogel) described in the present disclosure. In some embodiments, the SBP is a solution. In some embodiments, the SBP is a hydrogel.

### Lubricants

In some embodiments, processed silk and/or SBPs may be used as a lubricant. In some embodiments, processed silk may be selected base on or prepared to maximize its use as a lubricant. As used herein, the term “lubricant” refers to a substance that reduces the friction between two or more surfaces. In some embodiments, the surfaces in need of lubrication may be part of a subject. In some embodiments, surfaces in need of lubrication include, but are not limited to, the body, eyes, skin, scalp, mouth, vagina, nose, hands, feet, and lips. In some embodiments, SBPs are used for ocular lubrication. As used herein, the term “ocular lubrication” refers to a method of the reduction of friction and/or irritation in the eye. In some embodiments, processed silk and/or SBPs may be used to reduce friction caused by dryness, as taught in U.S. Pat. No. 9,907,836 (the content of which is herein incorporated by reference in its entirety). This dryness may be dryness in the eye. In some embodiments, SBPs are used as a lubricant in other therapeutic applications such as, but not limited to, nasal spray, eye drops, ear drops, vaginal creams, etc. In some embodiments, the coefficient of friction of an SBP is approximately that of naturally occurring, biological and/or protein lubricants (e.g. lubricin). In some embodiments, SBPs may be incorporated into a lubricant. Such methods may include any of those presented in International Publication No. WO2013163407, the contents of which are herein incorporated by reference in their entirety. In some embodiments, processed silk and/or SBPs may be used as an excipient. In some embodiments, processed silk and/or SBPs may be used as an excipient to prepare a lubricant.

### Gastrointestinal Indications

In some embodiments, therapeutic indications include gastrointestinal indications. As used herein, the term “gastrointestinal indication” refers to any disease, disorder, or condition related to the stomach and/or intestines. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. Non-limiting examples of gastrointestinal indications may include, but are not limited to, any of those listed in Table 5, above.

### Genetic Indications

In some embodiments, therapeutic indications include genetic indications. As used herein, the term “genetic indication” refers to any therapeutic indication that relates to or results from DNA mutation or dysfunctional DNA synthesis, replication, or repair. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. In some embodiments, genetic indications may include, but are not limited to, any of those listed in Table 5, above.

### Infectious Diseases

In some embodiments, SBPs may be used to treat therapeutic indications related to infectious agents. As used herein, the term “infectious agent” refers to any organism or virus that can invade or otherwise associate with a host and be spread between hosts. As used herein, the term “infectious disease” refers to any disorder or abnormal condition caused by an infectious agent.

Treatment of infectious diseases in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation.

Non-limiting examples of infectious agents include bacteria, viruses, fungi, and parasites. Infectious diseases may include or be caused by Acute bacterial rhinosinusitis, 14-day measles, Acne, Acrodermatitis chronica atrophicans (ACA)-(late skin manifestation of latent Lyme disease), Acute hemorrhagic conjunctivitis, Acute hemorrhagic cystitis, Acute rhinosinusitis, Adult T-cell Leukemia-Lymphoma (ATLL), African Sleeping Sickness, AIDS (Acquired Immunodeficiency Syndrome), Alveolar hydatid, Amebiasis, Amebic meningoencephalitis, Anaplasmosis, Anthrax, Arboviral or parainfectious, Ascariasis—(Roundworm infections), Aseptic meningitis, Athlete's foot (Tinea pedis), Australian tick typhus, Avian Influenza, Babesiosis, Bacillary angiomatosis, Bacterial meningitis, Bacterial vaginosis, Balanitis, Balantidiasis, Bang's disease, Barmah Forest virus infection, Bartonellosis (Verruga peruana; Carrion's disease; Oroya fever), Bat Lyssavirus Infection, Bay sore (Chiclero's ulcer), Baylisascaris infection (Racoon roundworm infection), Beaver fever, Beef tapeworm, Bejel (endemic syphilis), Biphasic meningoencephalitis, Black Bane, Black death, Black piedra, Blackwater Fever, Blastomycosis, Blennorrhea of the newborn, Blepharitis, Boils, Bornholm disease (pleurodynia), Borrelia miyamotoi Disease, Botulism, Boutonneuse fever, Brazilian purpuric fever, Break Bone fever, Brill, Bronchiolitis, Bronchitis, Brucellosis (Bang's disease), Bubonic plague, Bullous impetigo, Burkholderia mallei (Glanders), Burkholderia pseudomallei (Melioidosis), Buruli ulcers (also Mycoburuli ulcers). Busse, Busse-Buschke disease (Cryptococcosis), California group encephalitis, Campylobacteriosis, Candidiasis, Canefield fever (Canicola fever; 7-day fever; Weil's disease; leptospirosis; canefield fever), Canicola fever, Capillariasis, Carate, Carbapenem-resistant Enterobacteriaceae (CRE), Carbuncle, Carrion's disease, Cat Scratch fever, Cave disease, Central Asian hemorrhagic fever, Central European tick, Cervical cancer, Chagas disease, Chancroid (Soft chancre), Chicago disease, Chickenpox (Varicella), Chiclero's ulcer, Chikungunya fever, Chlamydial infection, Cholera, Chromoblastomycosis, Ciguatera, Clap, Clonorchiasis (Liver fluke infection), Clostridium difficile Infection, Clostridium perfringens (Epsilon Toxin), Coccidioidomycosis fungal infection (Valley fever; desert rheumatism), Coenurosis, Colorado tick fever, Condyloma accuminata, Condyloma accuminata (Warts), Condyloma lata, Congo fever, Congo hemorrhagic fever virus, Conjunctivitis, cowpox, Crabs, Crimean, Croup, Cryptococcosis, Cryptosporidiosis (Crypto), Cutaneous Larval Migrans, Cyclosporiasis, Cystic hydatid, Cysticercosis, Cystitis, Czechoslovak tick, D68 (EV-D68), Dacryocytitis, Dandy fever, Darling's Disease, Deer fly fever, Dengue fever (1, 2, 3 and 4), Desert rheumatism, Devil's grip, Diphasic milk fever, Diphtheria, Disseminated Intravascular Coagulation, Dog tapeworm, Donovanosis, Donovanosis (Granuloma inguinale), Dracontiasis, Dracunculosis, Duke's disease, Dum Dum Disease, Durand-Nicholas-Favre disease, Dwarf tapeworm, E. coli infection (E. coli), Eastern equine encephalitis, Ebola Hemorrhagic Fever (Ebola virus disease EVD), Ectothrix, Ehrlichiosis (Sennetsu fever), Encephalitis, Endemic Relapsing fever, Endemic syphilis, Endophthalmitis, Endothrix, Enterobiasis (Pinworm infection), Enterotoxin-B Poisoning (Staph Food Poisoning), Enterovirus Infection, Epidemic Keratoconjunctivitis, Epidemic Relapsing fever, Epidemic typhus, Epiglottitis, Erysipelis, Erysipeloid (Erysipelothricosis), Erythema chronicum migrans, Erythema infectiosum, Erythema marginatum, Erythema multiforme, Erythema nodosum, Erythema nodosum leprosum, Erythrasma, Espundia, Eumycotic mycetoma, European blastomycosis, Exanthem subitum (Sixth disease), Eyeworm, Far Eastern tick, Fascioliasis, Fievre boutonneuse (Tick typhus), Fifth Disease (erythema infectiosum), Filatow-Dukes' Disease (Scalded Skin Syndrome; Ritter's Disease), Fish tapeworm, Fitz-Hugh-Curtis syndrome—Perihepatitis, Flinders Island Spotted Fever, Flu (Influenza), Folliculitis, Four Corners Disease, Four Corners Disease (Human Pulmonary Syndrome (HPS)), Frambesia, Francis disease, Furnculosis, Gas gangrene, Gastroenteritis, Genital Herpes, Genital Warts, German measles, Gerstmann-Straussler-Scheinker (GSS), Giardiasis, Gilchrist's disease, Gingivitis, Gingivostomatitis, Glanders, Glandular fever (infectious mononucleosis), Gnathostomiasis, Gonococcal Infection (Gonorrhea), Gonorrhea, Granuloma inguinale (Donovanosis), Guinea Worm, Haemophilus Influenza disease, Hamburger disease, Hansen's disease—leprosy, Hantaan disease, Hantaan-Korean hemorrhagic fever, Hantavirus Pulmonary Syndrome, Hantavirus Pulmonary Syndrome (HPS), Hard chancre, Hard measles, Haverhill fever—Rat bite fever, Head and Body Lice, Heartland fever, Helicobacterosis, Hemolytic Uremic Syndrome (HUS), Hepatitis A, Hepatitis B, Hepatitis C, Hepatitis D, Hepatitis E Herpangina, Herpes—genital, Herpes labialis, Herpes—neonatal, Hidradenitis, Histoplasmosis, Histoplasmosis infection (Histoplasmosis), His-Werner disease, HIV infection, Hookworm infections, Hordeola, Hordeola (Stye), HTLV, HTLV-associated myelopathy (HAM), Human granulocytic ehrlichiosis, Human monocytic ehrlichiosis, Human Papillomavirus (HPV), Human Pulmonary Syndrome, Hydatid cyst, Hydrophobia, Impetigo, Including congenital (German Measles), Inclusion conjunctivitis, Inclusion conjunctivitis—Swimming Pool conjunctivitis—Pannus, Infantile diarrhea, Infectious Mononucleosis, Infectious myocarditis, Infectious pericarditis, Influenza, Isosporiasis, Israeli spotted fever, Japanese Encephalitis, Jock itch, Jorge Lobo disease—lobomycosis, Jungle yellow fever, Junin Argentinian hemorrhagic fever, Kala Azar, Kaposi's sarcoma, Keloidal blastomycosis, Keratoconjunctivitis, Kuru, Kyasanur forest disease, LaCrosse encephalitis, Lassa hemorrhagic fever, Legionellosis (Legionnaires Disease), Legionnaire's pneumonia. Lemierre's Syndrome (Postanginal septicemia), Lemming fever, Leprosy, Leptospirosis (Nanukayami fever; Weil's disease), Listeriosis (Listeria), Liver fluke infection, Lobo's mycosis, Lockjaw, Loiasis, Louping Ill, Ludwig's angina, Lung fluke infection, Lung fluke infection (Paragonimiasis), Lyme disease, Lymphogranuloma venereum infection (LGV), Machupo Bolivian hemorrhagic fever, Madura foot, Mal del pinto, Malaria, Malignant pustule, Malta fever, Marburg hemorrhagic fever, Masters disease, Maternal Sepsis (Puerperal fever), Measles, Mediterranean spotted fever, Melioidosis (Whitmore's disease), Meningitis, Meningococcal Disease, MERS, Milker's nodule, Molluscum contagiosum, Moniliasis, monkeypox, Mononucleosis. Mononucleosis-like syndrome, Montezuma's Revenge, Morbilli, MRSA (methicillin-resistant Staphylococcus aureus) infection, Mucormycosis-Zygomycosis, Multiple Organ Dysfunction Syndrome or MODS, Multiple-system atrophy (MSA), Mumps, Murine typhus, Murray Valley Encephalitis (MVE), Mycoburuli ulcers, Mycoburuli ulcers—Buruli ulcers, Mycotic vulvovaginitis, Myositis, Nanukayami fever, Necrotizing fasciitis, Necrotizing fasciitis—Type 1, Necrotizing fasciitis—Type 2, Negishi, New world spotted fever, Nocardiosis, Nongonococcal urethritis, Non-Polio (Non-Polio Enterovirus), Norovirus infection, North American blastomycosis, North Asian tick typhus, Norwalk virus infection, Norwegian itch, O'Hara disease, Omsk hemorrhagic fever, Onchoceriasis, Onychomycosis, Opisthorchiasis, Opthalmia neonatorium, Oral hairy leukoplakia, Orf, Oriental Sore, Oriental Spotted Fever, Ornithosis (Parrot fever; Psittacosis), Oroya fever, Otitis externa, Otitis media, Pannus, Paracoccidioidomycosis, Paragonimiasis, Paralytic Shellfish Poisoning (Paralytic Shellfish Poisoning), Paronychia (Whitlow), Parotitis, PCP pneumonia, Pediculosis, Peliosis hepatica, Pelvic Inflammatory Disease, Pertussis (also called Whooping cough), Phacohyphomycosis, Pharyngoconjunctival fever, Piedra (White Piedra), Piedra (Black Piedra), Pigbel, Pink eye conjunctivitis, Pinta, Pinworm infection. Pitted Keratolysis, Pityriasis versicolor (Tinea versicolor), Plague; Bubonic, Pleurodynia, Pneumococcal Disease, Pneumocystosis, Pneumonia, Pneumonic (Plague), Polio or Poliomyelitis. Polycystic hydatid, Pontiac fever, Pork tapeworm, Posada-Wernicke disease, Postanginal septicemia, Powassan, Progressive multifocal leukencephalopathy, Progressive Rubella Panencephalitis, Prostatitis, Pseudomembranous colitis, Psittacosis, Puerperal fever, Pustular Rash diseases (Small pox), Pyelonephritis, Pylephlebitis, Q-Fever, Quinsy, Quintana fever (5-day fever), Rabbit fever, Rabies, Racoon roundworm infection, Rat bite fever, Rat tapeworm, Reiter Syndrome, Relapsing fever, Respiratory syncytial virus (RSV) infection, Rheumatic fever, Rhodotorulosis, Ricin Poisoning, Rickettsialpox, Rickettsiosis, Rift Valley Fever, Ringworm, Ritter's Disease, River Blindness, Rocky Mountain spotted fever, Rose Handler's disease (Sporotrichosis), Rose rash of infants, Roseola, Ross River fever, Rotavirus infection, Roundworm infections, Rubella, Rubeola, Russian spring, Salmonellosis gastroenteritis, San Joaquin Valley fever, Sao Paulo Encephalitis, Sao Paulo fever, SARS, Scabies Infestation (Scabies) (Norwegian itch), Scalded Skin Syndrome, Scarlet fever (Scarlatina), Schistosomiasis, Scombroid, Scrub typhus, Sennetsu fever, Sepsis (Septic shock), Severe Acute Respiratory Syndrome, Severe Acute Respiratory Syndrome (SARS), Shiga Toxigenic Escherichia coli (STEC/VTEC), Shigellosis gastroenteritis (Shigella), Shinbone fever, Shingles, Shipping fever, Siberian tick typhus, Sinusitis, Sixth disease, Slapped cheek disease, Sleeping sickness, Smallpox (Variola), Snail Fever, Soft chancre, Southern tick associated rash illness, Sparganosis, Spelunker's disease, Sporadic typhus, Sporotrichosis, Spotted fever, Spring, St. Louis encephalitis, Staphylococcal Food Poisoning, Staphylococcal Infection, Strep. throat, Streptococcal Disease, Streptococcal Toxic-Shock Syndrome, Strongyloiciasis, Stye, Subacute Sclerosing Panencephalitis, Subacute Sclerosing Panencephalitis (SSPE), Sudden Acute Respiratory Syndrome, Sudden Rash, Swimmer's ear, Swimmer's Itch, Swimming Pool conjunctivitis, Sylvatic yellow fever, Syphilis, Systemic Inflammatory Response Syndrome (SIRS), Tabes dorsalis (tertiary syphilis), Taeniasis, Taiga encephalitis, Tanner's disease, Tapeworm infections. Temporal lobe encephalitis. Temporal lobe encephalitis, tetani (Lock Jaw), Tetanus Infection, Threadworm infections, Thrush, Tick, Tick typhus, Tinea barbac, Tinea capitis, Tinea corporis, Tinea cruis, Tinea manuum, Tinea nigra, Tinea pedis, Tinea unguium, Tinea versicolor, Torulopsosis, Torulosis, Toxic Shock Syndrome, Toxoplasmosis, transmissible spongioform (CJD), Traveler's diarrhea, Trench fever 5, Trichinellosis, Trichomoniasis, Trichomycosis axillaris, Trichuriasis, Tropical Spastic Paraparesis (TSP), Trypanosomiasis, Tuberculosis (TB), Tuberculosis, Tularemia, Typhoid Fever, Typhus fever, Ulcus molle, Undulant fever, Urban yellow fever, Urethritis. Vaginitis, Vaginosis, Vancomycin Intermediate (VISA), Vancomycin Resistant (VRSA), Varicella, Venezuelan Equine encephalitis, Verruga peruana, Vibrio cholerac (Cholera), Vibriosis (Vibrio), Vincent's disease or Trench mouth, Viral conjunctivitis, Viral Meningitis, Viral meningoencephalitis, Viral rash, Visceral Larval Migrans, Vomito negro, Vulvovaginitis, Warts. Waterhouse, Weil's disease, West Nile Fever, Western equine encephalitis, Whipple's disease, Whipworm infection, White Piedra, Whitlow, Whitmore's disease, Winter diarrhea, Wolhynia fever, Wool sorters' disease, Yaws, Yellow Fever, Yersinosis, Yersinosis (Yersinia), Zahorsky's disease, Zika virus disease, Zoster, Zygomycosis, John Cunningham Virus (JCV), Human immunodeficiency virus (HIV), Influenza virus, Hepatitis B, Hepatitis C, Hepatitis D, Respiratory syncytial virus (RSV), Herpes simplex virus 1 and 2, Human Cytomegalovirus, Epstein-Barr virus, Varicella zoster virus, Coronaviruses, Poxviruses, Enterovirus 71, Rubella virus, Human papilloma virus, Streptococcus pneumoniae, Streptococcus viridans; Staphylococcus aureus (S. aureus), Methicillin-resistant Staphylococcus aureus (MRSA), Vancomycin-intermediate Staphylococcus aureus (VISA), Vancomycin-resistant Staphylococcus aureus (VRSA), Staphylococcus epidermidis (S. epidermidis), Clostridium tetani, Bordetella pertussis, Bordetella paratussis, Mycobacterium, Francisella tularensis, Toxoplasma gondi, and/or Candida (C. albicans, C. glabrata, C. parapsilosis, C. tropicalis, C. krusei and C. lusitaniae), and/or any other infectious diseases, disorders or syndromes.

In some embodiments, infectious diseases result from exposure to various toxins produced by infectious agents. Such toxins may include, but are not limited to, Ricin, Bacillus anthracis, Shiga toxin, Shiga-like toxin, and Botulinum toxins. SBPs may be used to treat such infectious diseases.

In some embodiments, infectious agents may include, but are not limited to, adenoviruses, Anaplasma phagocytophilium, Ascaris lumbricoides, Bacillus anthracis, Bacillus cereus, Bacteroides sp. Barmah Forest virus, Bartonella bacilliformis, Bartonella henselae, Bartonella quintana, beta-toxin of Clostridium perfringens, Bordetella pertussis, Bordetella parapertussis, Borrelia burgdorferi, Borrelia miyamotoi, Borrelia recurrentis, Borrelia sp., Botulinum toxin, Brucella sp., Burkholderia pseudomallei, California encephalitis virus, Campylobacter, Candida albicans, chikungunya virus, Chlamydia psittaci, Chlamydia trachomatis, Clonorchis sinensis, Clostridium difficile bacteria, Clostridium tetani, Colorado tick fever virus, Corynebacterium diphtheriae, Corynebacterium minutissimum, Coxiella burnetii, coxsackie A, coxsackie B, Crimean-Congo hemorrhagic fever virus, cytomegalovirus, dengue virus, Eastern Equine encephalitis virus, Ebola viruses, echovirus, Ehrlichia chaffeensis, Ehrlichia equi, Ehrlichia sp., Entamoeba histolytica, Enterobacter sp., Enterococcus faecalis, Enterovirus 71, Epstein-Barr virus (EBV), Erysipelothrix rhusiopathiae, Escherichia coli, Flavivirus, Fusobacterium necrophorum, Gardnerella vaginalis, Group B streptococcus, Haemophilus aegyptius, Haemophilus ducreyi, Haemophilus influenzae, hantavirus, Helicobacter pylori, Hepatitis A, Hepatitis B, Hepatitis C, Hepatitis D, Hepatitis E, herpes simplex virus 1 and 2, human herpes virus 6, human herpes Virus 8, human immunodeficiency virus 1 and 2, human T-cell leukemia viruses I and II, influenza viruses (A, B, C), Jamestown Canyon virus, Japanese encephalitis antigenic, Japanese encephalitis virus, John Cunninham virus, juninvirus, Kaposi's Sarcoma-associated Herpes Virus (KSHV), Klebsiella granulomatis, Klebsiella sp., Kyasanur Forest Disease virus, La Crosse virus, Lassavirus, Legionella pneumophila, Leptospira interrogans, Listeria monocytogenes, lymphocytic choriomeningitis virus, lyssavirus, Machupovirus, Marburg virus, measles virus, MERS coronavirus (MERS-CoV), Micrococcus sedentarius, Mobiluncus sp., Molluscipoxvirus, Moraxella catarrhalis, Morbilli-Rubeola virus, Mumpsvirus, Mycobacterium leprae, Mycobacterium tuberculosis, Mycobacterium ulcerans, Mycoplasma genitalium, Mycoplasma sp, Nairovirus, Neisseria gonorrhoeae, Neisseria meningitidis, Nocardia, Norwalk virus, norovirus, Omsk hemorrhagic fever virus, papilloma virus, parainfluenza viruses 1-3, parapoxvirus, parvovirus B19, Peptostreptococccus sp., Plasmodium sp., polioviruses types I, II, and III, Proteus sp., Pseudomonas aeruginosa, Pseudomonas pseudomallei, Pseudomonas sp., rabies virus, respiratory syncytial virus, ricin toxin, Rickettsia australis, Rickettsia conori, Rickettsia honei, Rickettsia prowazekii, Ross River Virus, rotavirus, rubellavirus, Saint Louis encephalitis, Salmonella Typhi, Sarcoptes scabiei, SARS-associated coronavirus (SARS-CoV), Serratia sp., Shiga toxin and Shiga-like toxin, Shigella sp., Sin Nombre Virus, Snowshoe hare virus, Staphylococcus aureus, Staphylococcus epidermidis, Streptobacillus moniliformis, Streptococcus pneumoniae, Streptococcus agalactiae, Streptococcus agalactiae, Streptococcus group A-H, Streptococcus pneumoniae, Streptococcus pyogenes, Treponema pallidum subsp. Pallidum, Treponema pallidum var. carateum, Treponema pallidum var. endemicum, Tropheryma whippelii, Ureaplasma urcalyticum, Varicclla-Zoster virus, Variola virus, Vibrio cholerae, West Nile virus, yellow fever virus, Yersinia enterocolitica, Yersinia pestis, and Zika virus. Some SBPs may be used to treat infectious diseases caused by such infectious agents.

In some embodiments, therapeutic indications include any of the infectious indications listed in Table 5, above, or therapeutic indications resulting from exposure to any of the infectious agents listed in Table 5, above.

### Inflammatory Indications

In some embodiments, therapeutic indications include inflammatory indications. As used herein, the term “inflammatory indication” refers to a therapeutic indication that involves activation of the immune system. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation.

In some embodiments, inflammatory indications include one or more of joint disease, ophthalmic disease, retinal disease, psoriasis, Crohn's disease, irritable bowel syndrome, Sjogren's disease, tissue graft rejection, asthma, systemic lupus erythematosus, glomerulonephritis, dermatomyositis, multiple sclerosis, scleroderma, vasculitis, Goodpasture's syndrome, atherosclerosis, chronic idiopathic thrombocytopenic purpura, Addison's disease, Parkinson's disease, Alzheimer's disease, diabetes, septic shock, myasthenia gravis, inflammatory pelvic disease, inflammatory bowel disease, urethritis, uveitis, sinusitis, pneumonitis, encephalitis, meningitis, myocarditis, nephritis, osteomyelitis, myositis, hepatitis, gastritis, enteritis, dermatitis, appendicitis, pancreatitis, cholocystitis, polycystic kidney disease, and cancer. Inflammatory indications related to joint disease may include one or more of osteoarthritis, rheumatoid arthritis, spondyloarthritis, systemic juvenile idiopathic arthritis, psoriatic arthritis, gout, ankylosing spondylitis, and juvenile rheumatoid arthritis. In some embodiments, subjects treated for inflammatory indications have previously been diagnosed with an inflammatory indication.

In some embodiments, inflammatory indications include implant rejection. “Implant rejection” refers to an inflammatory condition caused by host immune response to material included in an implant. Treatment of implant rejection may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. In some embodiments, implant rejection may be prevented by using implants that are SBPs or that may be coated with SBPs. In some embodiments, SBP biocompatibility may prevent immune responses associated with implant rejection.

Additional inflammatory indications may include, but are not limited to, any of those listed in Table 5, above.

### Allergies

In some embodiments, therapeutic indications include allergies. As used herein, the term “allergy” refers to a hypersensitive immune response to one or more environmental stimulants. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation.

Examples of allergies include, but are not limited to, food allergies, skin allergies, dust allergies, insect allergies, pet allergies, eye allergies, skin allergies, drug allergies, latex allergies, allergic rhinitis, mold allergies, sinus infection, cockroach allergies, hay fever, pollen allergies, sinusitis, asthma, insect sting or venom allergies, skin contact allergies, eczema, dermatitis, allergic conjunctivitis, and chemical sensitivities. In some embodiments, allergies may include any of those listed in Table 5, above.

### Metabolic Indications

In some embodiments, therapeutic indications include metabolic indications. As used herein, the term “metabolic indication” refers to any therapeutic indication related to or resulting from dysfunctional metabolism. Metabolism refers collectively to bodily, cellular, and/or chemical processes responsible for maintaining life in living organisms. Treatment of metabolic indications in subjects may include contacting subjects with SBPs. SBPs may include therapeutic agents (e.g., insulin or any other therapeutic agents described herein) as cargo or payloads for treatment. In some embodiments, payload release may occur over a period of time (the payload release period). The payload release rate and/or length of the payload release period may be modulated by SBP components or methods of preparation.

Metabolic indications may include obesity or obesity-related indications. Non-limiting examples of obesity-related indications include, but are not limited to, cancer, heart disease, diabetes, Cushing's disease, polycystic ovary syndrome, hypertension, dyslipidemia, stroke, gallbladder disease, osteoarthritis, sleep apnea, breathing problems, depression, anxiety, and pain.

In some embodiments, the metabolic indications may be treated via enzyme replacement therapy. In some embodiments, SBPs described herein may be utilized to facilitate the delivery of components of enzyme replacement therapy. Enzyme replacement therapy provides therapeutic interventions that address an underlying metabolic defect in many disorders caused by defective enzymes. Such disorders include, but are not limited to, lysosomal storage diseases (LSDs), congenital disorders of glycosylation, and metabolic disorders characterized by missing or reduced enzyme activity in the cytoplasm. Non-limiting examples of lysosomal storage diseases include: Activator Deficiency; Alpha-mannosidosis, Aspartylglucosaminuria, Cholesteryl ester storage disease, Chronic Hexosaminidase A Deficiency, Cystinosis, Danon disease, Gaucher disease, Fabry disease, Farber disease; Fucosidosis; Galactosialidosis, GM gangliosidosis, I-Cell disease. Infantile Free Sialic Acid Storage Disease, Krabbe disease, Metachromatic Leukodystrophy, Pompe disease. Mucopolysaccharidosis I, Hurler syndrome. Hurler-Scheie syndrome, Scheie syndrome, Mucopolysaccharidosis II, Hunter syndrome, Mucopolysaccharidosis IV, Mucopolysaccharidosis VI, Lysosomal Acid lipase deficiency. Thrombocytopenia, Maroteaux-Lamy syndrome. Sly syndrome, Pycnodysostosis, Sandhoff disease, Schindler disease, Salla disease, Tay-Sachs, and Wolman disease.

In some embodiments, metabolic indications may include any of those listed in Table 5, above.

### Ocular Indications

In some embodiments, therapeutic indications include ocular indications. As used herein, the term “ocular indication” refers to any therapeutic indication related to the eye. In some embodiments, the therapeutic indication is an ophthalmology or ophthalmology-related disease and/or disorder. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. In some embodiments, SBPs may be provided in the form of a solution or may be incorporated into a solution for ocular administration. Such solutions may be administered topically (e.g., in the form of drops, creams, or sprays) or by injection. In some embodiments, SBPs may be provided in the format of a lens or may be incorporated into lenses that are placed on eye. In some embodiments. SBPs are provided in the form of implants or are incorporated into implants that may be placed around the eye, on a surface of the eye, in a periocular space or compartment, or intraocularly. Implants may be solid or gelatinous (e.g., a gel or slurry) and may be in the form of a bleb, rod, or plug. Some gelatinous implants may harden after application. In some embodiments, implants include punctal plugs. Such plugs may be inserted into tear ducts. In some embodiments, SBPs may be used to repair ocular damage. In some embodiments, the SBP adheres to the ocular surface. In some embodiments, the SBP adheres to the ocular surface in a manner similar to a mucin layer. Intravitreal administration may be performed at any injection site that would enable the administration of the SBP to the intravitreal space.

Non-limiting examples of ocular indications include infection, refractive errors, age related macular degeneration, cystoid macular edema, cataracts, diabetic retinopathy (proliferative and non-proliferative), glaucoma, amblyopia, strabismus, color blindness, cytomegalovirus retinitis, keratoconus, diabetic macular edema (proliferative and non-proliferative), low vision, ocular hypertension, retinal detachment, eyelid twitching, inflammation, uveitis, bulging eyes, dry eye disease, floaters, xerophthalmia, diplopia, Graves' disease, night blindness, eye strain, red eyes, nystagmus, presbyopia, excess tearing, retinal disorders (e.g. age related macular degeneration), conjunctivitis, cancer, corneal ulcer, corneal abrasion, snow blindness, scleritis, keratitis, Thygeson's superficial punctate keratopathy, corneal neovascularization, Fuch's dystrophy, keratoconjuctitivis sicca, iritis, chorioretinal inflammation (e.g. chorioretinitis, choroiditis, retinitis, retinochoroiditis, pars planitis, and Harada's disease), aniridia, macular scars, solar retinopathy, choroidal degeneration, choroidal dystrophy, choroideremia, gyrate atrophy, choroidal hemorrhage, choroidal detachment, retinoschisis, hypertensive retinopathy. Bull's eye maculopathy, epiretinal membrane, peripheral retinal degeneration, hereditary retinal dystrophy, retinitis pigmentosa, retinal hemorrhage, separation of retinal layers, retinal vein occlusion, and other visual impairments. In some embodiments, ocular indications include inflammation of the eye.

Ocular indications may include dry eye. Dry eye is a condition involving a lack of hydration on the eye surface that may be caused by one or more of a variety of factors (e.g., cellular/tissue dysfunction or environmental irritants). In some embodiments, SBPs used to treat dry eye are provided as or included in solutions or devices. Solutions may be administered topically (e.g., by cream, spray, or drops) or by injection to periocular or intraocular areas. Solutions may include viscous solutions, such as gels or slurries. Devices may include, but are not limited to, implants, lenses, and plugs. Devices may be hardened structures or gelatinous. In some embodiments, devices are gelatinous, but harden after placement. Devices may include lacrimal or punctal plugs that treat dry eye via tear duct insertion. SBPs used to treat dry eye may include therapeutic agent payloads. The therapeutic agents may include any of those described herein. In some embodiments, therapeutic agents include one or more of cyclosporine, corticosteroids, tetracyclines, and essential fatty acids. Therapeutic agent release from SBPs may occur over an extended payload release period. The payload release period may be from about 1 hour to about 48 hours, from about 1 day to about 14 days, or from about 1 week to about 52 weeks, or more than 52 weeks. In some embodiments, ocular SBPs may be used as an anti-inflammatory treatment for dry eye disease, as described in Kim el al. (2017) Scientific Reports 7: 44364, the contents of which are herein incorporated by reference in their entirety. It has been demonstrated that the administration of 0.1 to 0.5% silk fibroin solutions in a mouse model of dry eye disease enhances corneal smoothness and tear production, while reducing the amount of inflammatory markers detected.

Ocular indications may include diabetic retinopathy. The term “diabetic retinopathy” refers to the damage to the blood vessels in the back of the eye caused by complications of diabetes. Both type I and type II diabetes can lead to diabetic retinopathy. The early stages of the indication, known as non-proliferative diabetic retinopathy, include weakened blood vessels and microaneurysms. The later stages of the indication, known as proliferative diabetic retinopathy, may lead to a lack of circulation in the retina and improper blood vessel growth.

Ocular indications may include diabetic macular edema. The term “diabetic macular edema” refers to an accumulation of the fluid in the macula, the area of the eye responsible for high-resolution central vision. Diabetic macular edema may be caused by diabetic retinopathy. Treatments for diabetic macular edema may include VEGF-related agents (e.g. antibodies or antagonists), and steroids (e.g. triamcinolone).

Ocular indications may include glaucoma. The term “glaucoma” refers to a group of ocular disorders that cause optic nerve damage, sometimes leading to loss of vision or blindness. Glaucoma is often associated with elevated intraocular pressure. The pressure may be caused by inefficient drainage of intraocular fluid. The optic nerve is sensitive to intraocular pressure and increased pressure can lead to damage. “Refractory glaucoma” refers to glaucoma that persists or is at risk to persist after attempts to reduce intraocular pressure (e.g., surgical intervention).

In some embodiments, ocular indications may include post-operative cystoid macular edema (CME). In some embodiments, ocular indications may include age-related macular degeneration (AMD), whether wet or dry. In some embodiments, ocular indications may include diabetic macular edema (DME). Additional ocular indications may include, but are not limited to, any of those listed in Table 5, above.

### Otorhinolaryngological Indications

In some embodiments, therapeutic indications include otorhinolaryngological indications. As used herein, the term “otorhinolaryngological indication” refers to any disease, disorder, or condition related to the ear, nose, and/or throat. In some embodiments, the therapeutic indication is an otology or an otology-related disease and/or disorder. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. Non-limiting examples of gastrointestinal indications may include, but are not limited to, any of those listed in Table 5, above.

In some embodiments, therapeutic indications include hearing disorders. As used herein, the term “hearing disorder” refers to any disease, disorder, or condition related to the impairment of the sense of hearing. Hearing disorder may include a broad range of indications, including, but not limited to, genetic hearing loss, age-related hearing loss, noise-induced hearing loss hearing loss, tinnitus, and drug-induced ototoxicity. Treatment of such indications in subjects may include contacting subjects with SBPs. SBPs may include therapeutic agents (e.g., any of those described herein) as cargo or payloads for treatment. In some embodiments, SBPs may be used to formulate an API (e.g., a small molecule, a peptide, a viral particle, or any other biologic, etc.) for the treatment of the hearing disorder. Alternatively, SBPs may be used in the fabrication, production, and/or manufacture of a hearing aid device. Further, SBPs may also be used for cochlear implants or ear drum tissue engineering.

### Pain

In some embodiments, therapeutic indications include pain. Pain treatments may include contacting subjects with SBPs. SBPs may include therapeutic agents (e.g., any of those described herein) as cargo or payloads for treatment. In some embodiments, the payload is a pain killer (e.g., see United States Publication Number US20050149119 or International Publication Number WO2017139684, the contents of each of which are herein incorporated by reference in their entirety). In some embodiments, payload release may occur over a period of time (the payload release period). The payload release rate and/or length of the payload release period may be modulated by SBP components or methods of preparation.

Different types and levels of pain may be treated using SBPs. In some embodiments, pain includes one or more of nociceptive pain, neuropathic pain, psychogenic pain, breakthrough pain, incident pain, back pain, musculoskeletal pain, post-operative pain, operative pain, visceral pain, joint pain, acute pain, inflammatory pain, knee pain, dental pain, and chronic pain. Additional forms of pain may include, but are not limited to, any of those listed in Table 5, above.

In some embodiments, pain treatment using SBPs may lead to pain reduction. Changes in pain levels due to SBP treatments may be assessed using a pain scale. Non-limiting examples of pain scales for measuring pain intensity include Alder Hey Triage Pain Score, Behavioral Pain Scale (BPS), Brief Pain Inventory (BPI), Checklist of Nonverbal Pain Indicators (CNPI), Clinical Global Impression (CGI), Critical-Care Point Observation Tool (CPOT), COMFORT scale, Dallas Pain Questionnaire, Descriptor Differential Scale (DDS), Dolorimeter Pain Index (DPI), Edmonton Symptom Assessment System, Faces Pain Scale-Revised (FPS-R), Face Legs Activity Cry Consolability Scale, Lequesne Algofunctional Index, McGill Pain Questionnare (MPQ), Neck Pain and Disability Scale (NPAD), Numerical 11 Point Box (BS-11), Numeric Rating Scale (NRS-11), OSWESTRY Index, Palliative Care Outcome Scale (PCOS), Roland Morris Back Pain Questionnare, Support Team Assessment Schedule (STAS), WongBaker FACES Pain Rating Scale, Visual Analog Scale (VAS), Australian/Canadian Osteoarthritis Hand Index (AUSCAN), Western Ontario and McMaster Universities Hand Index (WOMAC), and Osteoarthritis Research Society International-Outcome Measures in Rheumatoid Arthritis Clinical Trials (OARSI-OMERA).

In some embodiments, SBPs may be used to relieve osteoarthritis pain for an extended time, for example, for at least 5 days, at least 10 days, at least 15 days, at least 20 days, at least 25 days, at least 30 days, at least 35 days, at least 40 days, at least 45 days, or at least 50 days.

In some embodiments, SBPs used to for the treatment of pain (e.g., osteoarthritis) contain processed silk as the active therapeutic component.

### Psychological Indications

In some embodiments, therapeutic indications include psychological indications. As used herein, the term “psychological indication” refers to any disease, disorder, or condition that affects or is related to the mind and/or a subject's mental state. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation.

Non-limiting examples of psychological indications include Aboulia, Absence epilepsy, Acute stress Disorder, Adjustment Disorders, Adverse effects of medication NOS, Age related cognitive decline, Agoraphobia, Alcohol Addiction, Alzheimer's Disease, Amnesia (also known as Amnestic Disorder). Amphetamine Addiction, Anorexia Nervosa. Anterograde amnesia. Antisocial personality disorder (also known as Sociopathy), Anxiety Disorder (Also known as Generalized Anxiety Disorder), Anxiolytic related disorders, Asperger's Syndrome (now part of Autism Spectrum Disorder), Attention Deficit Disorder (Also known as ADD), Attention Deficit Hyperactivity Disorder (Also known as ADHD), Autism Spectrum Disorder (also known as Autism), Autophagia, Avoidant Personality Disorder, Barbiturate related disorders, Benzodiazepine related disorders, Bereavement, Bibliomania, Binge Eating Disorder, Bipolar disorder (also known as Manic Depression, includes Bipolar I and Bipolar II), Body Dysmorphic Disorder, Borderline intellectual functioning, Borderline Personality Disorder, Breathing-Related Sleep Disorder, Brief Psychotic Disorder. Bruxism, Bulimia Nervosa, Caffeine Addiction, Cannabis Addiction, Catatonic disorder, Catatonic schizophrenia, Childhood amnesia, Childhood Disintegrative Disorder (now part of Autism Spectrum Disorder). Childhood Onset Fluency Disorder (formerly known as Stuttering), Circadian Rhythm Disorders, Claustrophobia, Cocaine related disorders, Communication disorder, Conduct Disorder, Conversion Disorder, Cotard delusion, Cyclothymia (also known as Cyclothymic Disorder), Delerium, Delusional Disorder, dementia, Dependent Personality Disorder (also known as Asthenic Personality Disorder), Depersonalization disorder (now known as Depersonalization/Derealization Disorder), Depression (also known as Major Depressive Disorder), Depressive personality disorder, Derealization disorder (now known as Depersonalization/Derealization Disorder), Dermotillomania, Desynchronosis, Developmental coordination disorder, Diogenes Syndrome, Disorder of written expression, Dispareunia, Dissocial Personality Disorder, Dissociative Amnesia, Dissociative Fugue, Dissociative Identity Disorder (formerly known as Multiple Personality Disorder). Down syndrome, Dyslexia. Dyspareunia, Dysthymia (now known as Persistent Depressive Disorder), Eating disorder NOS, Ekbom's Syndrome (Delusional Parasitosis), Emotionally unstable personality disorder, Encopresis, Enuresis (bedwetting), Erotomania. Exhibitionistic Disorder, Expressive language disorder, Factitious Disorder, Female Sexual Disorders, Fetishistic Disorder, Folie à deux, Fregoli delusion, Frotteuristic Disorder, Fugue State, Ganser syndrome, Gambling Addiction, Gender Dysphoria (formerly known as Gender Identity Disorder), Generalized Anxiety Disorder, General adaptation syndrome, Grandiose delusions, Hallucinogen Addiction, Haltlose personality disorder, Histrionic Personality Disorder, Primary hypersomnia, Huntington's Disease, Hypoactive sexual desire disorder, Hypochondriasis, Hypomania, Hyperkinetic syndrome, Hypersomnia, Hysteria, Impulse control disorder, Impulse control disorder NOS, Inhalant Addiction, Insomnia, Intellectual Development Disorder, Intermittent Explosive Disorder, Joubert syndrome, Kleptomania, Korsakoff's syndrome, Lacunar amnesia, Language Disorder, Learning Disorders, Major Depression (also known as Major Depressive Disorder), major depressive disorder, Male Sexual Disorders, Malingering, Mathematics disorder, Medication-related disorder, Melancholia, Mental Retardation (now known as Intellectual Development Disorder), Misophonia, Morbid jealousy, Multiple Personality Disorder (now known as Dissociative Identity Disorder), Munchausen Syndrome, Munchausen by Proxy, Narcissistic Personality Disorder. Narcolepsy, Neglect of child, Neurocognitive Disorder (formerly known as Dementia), Neuroleptic-related disorder, Nightmare Disorder, Non Rapid Eye Movement, Obsessive-Compulsive Disorder, Obsessive-Compulsive Personality Disorder (also known as Anankastic Personality Disorder), Oneirophrenia, Onychophagia, Opioid Addiction, Oppositional Defiant Disorder, Orthorexia (ON), Pain disorder, Panic attacks, Panic Disorder, Paranoid Personality Disorder, Parkinson's Disease. Partner relational problem. Passive-aggressive personality disorder. Pathological gambling, Pedophilic Disorder, Perfectionism, Persecutory delusion, Persistent Depressive Disorder (also known as Dysthymia). Personality change due to a general medical condition, Personality disorder, Pervasive developmental disorder (PDD), Phencyclidine related disorder, Phobic disorder, Phonological disorder. Physical abuse, Pica, Polysubstance related disorder, Postpartum Depression, Post-traumatic embitterment disorder (PTED), Post-Traumatic Stress Disorder, Premature ejaculation, Premenstrual Dysphoric Disorder, Psychogenic amnesia, Psychological factor affecting medical condition, Psychoneurotic personality disorder, Psychotic disorder, not otherwise specified, Pyromania, Reactive Attachment Disorder, Reading disorder, Recurrent brief depression, Relational disorder, REM Sleep Behavior Disorder, Restless Leg Syndrome, Retrograde amnesia, Retts Disorder (now part of Autism Spectrum Disorder), Rumination syndrome, Sadistic personality disorder, Schizoaffective Disorder, Schizoid Personality Disorder, Schizophrenia, Schizophreniform disorder. Schizotypal Personality Disorder, Seasonal Affective Disorder. Sedative, Hypnotic, or Anxioytic Addiction, Selective Mutism, Self-defeating personality disorder, Separation Anxiety Disorder. Sexual Disorders Female, Sexual Disorders Male, Sexual Addiction. Sexual Masochism Disorder, Sexual Sadism Disorder, Shared Psychotic Disorder, Sleep Arousal Disorders, Sleep Paralysis, Sleep Terror Disorder (now part of Nightmare Disorder, Social Anxiety Disorder, Somatization Disorder, Specific Phobias. Stendhal syndrome, Stereotypic movement disorder, Stimulant Addiction. Stuttering (now known as Childhood Onset Fluency Disorder), Substance related disorder, Tardive dyskinesia, Tobacco Addiction, Tourettes Syndrome, Transient tic disorder, Transient global amnesia, Transvestic Disorder, Trichotillomania, Undifferentiated Somatoform Disorder, Vaginismus, and Voyeuristic Disorder. Additional psychological indications may include, but are not limited to, any of those listed in Table 5, above.

### Pulmonary Indications

In some embodiments, therapeutic indications include pulmonary indications. As used herein, the term “pulmonary indication” refers to any disease, disorder, or condition related to the lungs. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. Non-limiting examples of pulmonary indications may include, but are not limited to, any of those listed in Table 5, above.

### Rare Diseases

In some embodiments, SBPs and the methods described herein may be used to treat rare diseases. As used herein, the term “rare disease” refers to any disease that affects a small percentage of the population. As non-limiting examples, rare disease may include Acrocephalosyndactylia. Acrodermatitis, Addison Disease, Adie Syndrome, Alagille Syndrome, Amylose, Amyotrophic Lateral Sclerosis, Angelman Syndrome, Angiolymphoid Hyperplasia with Eosinophilia. Amold-Chiari Malformation, Arthritis, Juvenile Rheumatoid, Asperger Syndrome, Bardet-Biedl Syndrome. Barrett Esophagus. Beckwith-Wiedemann Syndrome, Behcet Syndrome, Bloom Syndrome, Bowen's Disease, Brachial Plexus Neuropathies, Brown-Sequard Syndrome, Budd-Chiari Syndrome, Burkitt Lymphoma, Carcinoma 256, Walker, Caroli Disease, Charcot-Marie-Tooth Disease, Chediak-Higashi Syndrome, Chiari-Frommel Syndrome, Chondrodysplasia Punctata, Colonic Pseudo-Obstruction, Colorectal Neoplasms, Hereditary Nonpolyposis, Craniofacial Dysostosis, Creutzfeldt-Jakob Syndrome, Crohn Disease, Cushing Syndrome, Cystic Fibrosis, Dandy-Walker Syndrome, De Lange Syndrome, Dementia, Vascular, Dermatitis Herpetiformis, DiGeorge Syndrome, Diffuse Cerebral Sclerosis of Schilder, Duane Retraction Syndrome. Dupuytren Contracture, Ebstein Anomaly, Eisenmenger Complex, Ellis-Van Creveld Syndrome, Encephalitis, Enchondromatosis, Epidermal Necrolysis, Toxic, Facial Hemiatrophy, Factor XII Deficiency, Fanconi Anemia, Felty's Syndrome, Fibrous Dysplasia, Polyostotic, Fox-Fordyce Disease, Friedreich Ataxia, Fusobacterium, Gardner Syndrome, Gaucher Disease, Gerstmann Syndrome, Giant Lymph Node Hyperplasia, Glycogen Storage Disease Type I, Glycogen Storage Disease Type II, Glycogen Storage Disease Type IV, Glycogen Storage Disease Type V, Glycogen Storage Disease Type VI, Goldenhar Syndrome, Guillain-Barre Syndrome, Hallermann's Syndrome, Hamartoma Syndrome, Multiple, Hartnup Disease, Hepatolenticular Degeneration, Hepatolenticular Degeneration, Hereditary Sensory and Motor Neuropathy, Hirschsprung Disease, Histiocytic Necrotizing Lymphadenitis, Histiocytosis, Langerhans-Cell, Hodgkin Disease, Homer Syndrome, Huntington Disease, Hyperaldosteronism, Hyperhidrosis, Hyperostosis, Diffuse Idiopathic Skeletal, Hypopituitarism, Inappropriate ADH Syndrome, Intestinal Polyps, Isaacs Syndrome, Kartagener Syndrome, Kearns-Sayre Syndrome, Klippel-Feil Syndrome, Klippel-Trenaunay-Weber Syndrome, Kluver-Bucy Syndrome, Korsakoff Syndrome, Lafora Disease, Lambert-Eaton Myasthenic Syndrome, Landau-Kleffner Syndrome, Langer-Giedion Syndrome, Leigh Disease, Lesch-Nyhan Syndrome, Leukodystrophy, Globoid Cell, Li-Fraumeni Syndrome, Long QT Syndrome, Machado-Joseph Disease, Mallory-Weiss Syndrome, Marek Disease, Marfan Syndrome, Meckel Diverticulum, Meige Syndrome, Melkersson-Rosenthal Syndrome. Meniere Disease, Mikulicz Disease. Miller Fisher Syndrome, Mobius Syndrome, Moyamoya Disease, Mucocutaneous Lymph Node Syndrome, Mucopolysaccharidosis I, Mucopolysaccharidosis II, Mucopolysaccharidosis III, Mucopolvsaccharidosis IV, Mucopolysaccharidosis VI, Multiple Endocrine Neoplasia Type 1, Munchausen Syndrome by Proxy, Muscular Atrophy, Spinal, Narcolepsy, Neuroaxonal Dystrophies, Neuromyelitis Optica, Neuronal Ceroid-Lipofuscinoses, Niemann-Pick Diseases, Noonan Syndrome, Optic Atrophies, Hereditary, Osteitis Deformans, Osteochondritis, Osteochondrodysplasias, Osteolysis, Osteoarthritis, Essential. Paget Disease Extramammary, Paget's Disease, Mammary, Panniculitis, Nodular Nonsuppurative, Papillon-Lefevre Disease, Paralysis, Pelizaeus-Merzbacher Disease, Pemphigus, Benign Familial, Penile Induration, Pericarditis, Constrictive, Peroxisomal Disorders. Peutz-Jeghers Syndrome, Pick Disease of the Brain, Pierre Robin Syndrome, Pigmentation Disorders, Pityriasis lichenoides, Polycystic Ovary Syndrome, Polvendocrinopathies, Autoimmune, Prader-Willi Syndrome, Pupil Disorders, Rett Syndrome, Reye Syndrome, Rubinstein-Taybi Syndrome, Sandhoff Disease, Sarcoma, Ewing's, Schnitzler Syndrome, Sjogren's Syndrome, Sjogren-Larsson Syndrome, Smith-Lemli-Opitz Syndrome, Spinal Muscular Atrophies of Childhood, Sturge-Weber Syndrome, Sweating, Gustatory, Takayasu Arteritis, Tangier Disease, Tay-Sachs Disease, Thromboangiitis Obliterans, Thyroiditis, Autoimmune, Tietze's Syndrome, Togaviridae Infections, Tolosa-Hunt Syndrome, Tourette Syndrome, Uveomeningoencephalitic Syndrome, Waardenburg's Syndrome. Wegener Granulomatosis, Weil Disease, Werner Syndrome, Williams Syndrome, Wilms Tumor, Wolff-Parkinson-White Syndrome, Wolfram Syndrome, Wolman Disease, Zellweger Syndrome, Zollinger-Ellison Syndrome, and von Willebrand Diseases.

Treatment of rare diseases in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation.

### Transplant-Related Indications

In some embodiments, therapeutic indications include transplant-related indications. As used herein, the term “transplant-related indication” refers to any condition related to transplantation (e.g. skin graft, organ transplant, etc.) of tissues, cells, and/or organs. Treatment of such indications in subjects may include contacting subjects and/or transplanted materials with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. Therapeutic agents used to treat transplant-related indications may include steroids, complement inhibitors, anti-inflammatory agents, gene therapy agents, or any other agents known to those skilled in the art for preventing transplant rejection.

In some embodiments, transplant-related indications include transplant rejection. Transplant rejection is a condition where the host immune system attacks the transplanted material. In some embodiments, transplant-related indications include graft versus host disease (GVHD). GVHD is a condition that arises after transplantation (e.g. skin graft, organ transplant, etc.) of tissues, cells, and/or organs, in which the immune system of the transplanted material may recognize the tissue and/or cells of the host as a foreign entity, and an immune response ensues.

Additional transplant-related indications may include, but are not limited to, any of those listed in Table 5, above.

### Vascular Indications

In some embodiments, therapeutic indications include vascular indications. As used herein, the term “vascular indication” refers to any disease, disorder, or condition that affects or is related to blood vessels. Treatment of such indications in subjects may include contacting subjects with SBPs. SBPs may include therapeutic agents (e.g., any of those described herein) as cargo or payloads for treatment. In some embodiments, payload release may occur over a period of time (the payload release period). The payload release rate and/or length of the payload release period may be modulated by SBP components or methods of preparation. In some embodiments, vascular indications may include, but are not limited to, any of those listed in Table 5, above.

### Veterinary Indications

In some embodiments, SBPs may be used to treat therapeutic indications affecting, prevalent in, or specific for non-human animals (referred to herein as “veterinary indications”). Veterinary indications may include any of the therapeutic indications presented previously in addition to those described below.

In some embodiments, veterinary indications may include infectious diseases. Such infectious diseases may include, but are not limited to Acute hepatopancreatic necrosis disease, Aflatoxicosis, African swine fever, Akabane, Anthrax, Australian bat lyssavirus, Avian influenza (bird flu), Avian paramyxovirus, Blue-green algae (cyanobacteria), Bluetongue, Botulism, Botulism in poultry, Bovine ephemeral fever, Bovine tuberculosis, Bovine virus diarrhea, Brucellosis, Brucella ovis, Buffalo fly, Campylobacteriosis (vibriosis), Caprine arthritis encephalitis (CAE), Cat-scratch disease, Cattle ticks, Classical swine fever, Clostridial diseases, Copper deficiency, Cryptococcosis, Enzootic bovine leucosis (EBL), Epizootic ulcerative syndrome (red-spot disease), Equine herpesvirus, Equine infectious anaemia (EIA), Equine influenza, Equine viral arteritis (EVA), Foot and mouth disease, Fowl cholera, Fowl pox, Giardiasis, Hendra virus, Hydatid disease (hydatid cysts), Infectious laryngotracheitis, Japanese encephalitis, Johne's disease, Leptospirosis, Listeriosis, Lumpy jaw, Marek's disease, Melioidosis, Neospora caninum, Newcastle disease, Nipah virus, Nosema, Ovine brucellosis, Pestivirus, Pimelea poisoning (St George disease, marree disease), Psittacosis (ornithosis), Q fever, Rabies, Rinderpest, Ringworm, Salmonellosis, Screw-worm fly, Skin fluke infestation, Sparganosis, Spotty liver, Strangles, African Swine fever, Classical Swine fever, Swine influenza, Swine vesicular disease, Tetanus, Tick fever, Toxocariasis, Toxoplasmosis, Transit tetany, Transmissible spongiform encephalopathies, Tuberculosis (TB), Vesicular exanthema, Vesicular stomatitis, Warts. White nose syndrome, White spot disease, and Wooden tongue (and lumpy jaw).

In some embodiments, veterinary indications may include some forms of cancer. Such cancers may include, but are not limited to, tumors, hematological malignancies, lymphomas, leukemias, carcinomas, and sarcomas. In some embodiments, cancers or tumors include those found in the anus, bladder, bile duct, bone, brain, breast, cervix, chest, colon/rectum, connective tissue, endometrium, esophagus, eye, gallbladder, head and neck, liver, kidney, larynx, lung, mouth, nose, ovaries, pancreas, penis, prostate, skin, small intestine, stomach, spinal marrow, tailbone, testicles, throat, thyroid and uterus.

In some embodiments, veterinary indications may include, but are not limited to, any of those listed in Table 5, above.

In one embodiment, the veterinary indication is dry eye.

### Gene Therapy

In some embodiments, therapeutic applications utilizing SBPs may include gene therapy. Gene therapy is revolutionizing medicine and offering new promise for the treatment of previously intractable conditions. As used herein, the term “gene therapy” refers to the use of genetic transplantation to address disease and/or genetic disorders. The transplantation may include substituting a defective gene with a non-defective gene or inserting a non-defective gene into one or more places in the genome. In some embodiments, SBPs may be used for gene therapy. Such SBPs may be used to facilitate the delivery of nucleic acids or vectors carrying nucleic acids. In some embodiments, SBPs are used to stabilize or preserve nucleic acids, nucleic acid delivery vehicles, or vectors used in gene therapy. Examples of genetic disorders that may be addressed by gene therapy include, but are not limited to, Achondroplasia, Alpha-1 Antitrypsin Deficiency, Antiphospholipid Syndrome, Autism, Autosomal Dominant Polycystic Kidney Disease, Breast cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy. Factor V Leiden Thrombophilia, Familial Hypercholesterolemia, Familial Mediterranean Fever, Fragile X Syndrome, Gaucher Disease, Hemochromatosis, Hemophilia, Holoprosencephaly, Huntington's disease, Klinefelter syndrome, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Tay-Sachs, Talassemia, Trimethylaminuria, Turner Syndrome, Velocardiofacial Syndrome, WAGR Syndrome, and Wilson Disease.

In some embodiments, the genetic disorder is a coagulation defect. Coagulation defects often cause hemorrhage and/or thrombosis. The best-known coagulation factor disorders are the hemophilias. The three main forms are hemophilia A (factor VIII deficiency), hemophilia B (factor IX deficiency or “Christmas disease”) and hemophilia C (factor XI deficiency, mild bleeding tendency). Other disorders caused by defective coagulation factors also include, but are not limited to, Von Willebrand disease (caused by a defect in von Willebrand factor (vWF), Bernard-Soulier syndrome (caused by a defect or deficiency in GPIb, a receptor of vWF), thrombophlebitis (caused by mutations in Factor XII), Congenital afibrinogenemia, Familial renal amyloidosis (caused by mutations in Factor I), congenital proconvertin/factor VII deficiency, Thrombophilia (caused by Factor II deficiency), Congenital Factor X deficiency, Congenital Factor XIIIa/b deficiency, Prekallikrein/Fletcher Factor deficiency, Kininogen deficiency, Glomerulopathy with fibronectin deposits, Heparin cofactor II deficiency, Protein C deficiency, Protein S deficiency, Protein Z deficiency, Antithrombin III deficiency, Plasminogen deficiency, type I (ligneous conjunctivitis), Antiplasmin deficiency, Plasminogen activator inhibitor-1 deficiency, and Quebec platelet disorder.

Gene therapy for coagulation factor replacement is a medical treatment of disorders caused by coagulation deficiency. In some embodiments, SBPs may be used to deliver and/or regulate gene therapy to replace coagulation factors. Such coagulation factors may include, but are not limited to, Factor I (fibrinogen), Factor II (prothrombin), Factor III (tissue factor). Factor IV, Factor V (proaccelerin), Factor VI, Factor VII (stable factor), Factor VIII (antihemophilic factor A), Factor IX (antihemophilic factor B), Factor X (Stuart-Prower factor), Factor XI (plasma thromboplastin antecedent), Factor XII (Hageman factor), Factor XIII (fibrin-stabilizing factor), von Willebrand factor, Prekallikrein (Fletcher factor), high-molecular-weight kininogen (HMWK) (Fitzgerald factor), fibronectin, antithrombin III, heparin cofactor II, protein C, protein S, protein Z, protein Z related protease inhibitor (ZPI), plasminogen, tissue plasminogen activator (tPA), urokiase, plasminogen, plasminogen activator inhibitor 1 (PAI1), and plasminogen activator inhibitor 2 (PAI2). In some embodiments, the coagulate factor is Factor VIII for gene therapy of hemophilia, including wild type factor VIII, engineered Factor VIII, activated fVIII (fVIIIa), or the equivalent.

### Gene Editing

In some embodiments, therapeutic applications utilizing SBPs may include gene editing. As used herein, the term “gene editing” refers to any process used to alter a DNA gene sequence at the level of individual nucleotides. Some methods of gene editing utilize CRISPR-Cas9 systems. CRISPR-Cas9 systems are a class of cutting edge genome editing systems developed and modified for use in genetic editing and proven to be highly effective and specific tools for editing nucleic acid sequences, even in eukaryotic cells. Various modifications to the bacterial CRISPR-Cas systems have been developed and demonstrated for use to manipulate nucleic acid in cells (e.g., mammalian and plant cells). Examples of CRISPR-Cas systems and methods of use are described in U.S. Pat. Nos. 8,993,233; 8,999,641; 8,945,839; 8,932,814; 8,906,616; 8,889,418; 8,889,356; 8,871,445; 8,865,406; 8,771,945; and 8,697,359; and United States Publication Numbers US20150031134; US20150203872; US20150218253; US20150176013; US20150191744; US20150071889; US20150067922; and US20150167000; the contents of each of which are herein incorporated by reference in their entirety.

In some embodiments, SBPs described herein may be used to stabilize or facilitate the delivery and/or controlled release of CRISPR-Cas9 system components needed for gene editing. In some embodiments, the CRISPR-Cas9 system component is the Cas9 enzyme, or alternative isoforms of the Cas9 enzyme, or orthologs of the Cas9 enzyme. The most commonly used Cas9 is derived from Streptococcus pyogenes and the RuvC domain can be inactivated by a DOA mutation and the HNH domain can be inactivated by an H840A mutation. Examples of Cas9 orthologs from other bacterial strains include, but are not limited to, Cas proteins identified in Acaryochloris marina MBIC11017; Acerohalobium arabaticum DSM 5501; Acidithiobacillus caldus; Acidithiobacillus ferrooxidans ATCC 23270; Alicyclobacillus acidocaldarius LA A1; Alicyclobacillus acidocaldanus subsp. acidocaldarius DSM 446; Allochromatium vinosum DSM 180; Ammonifex degensii KC4; Anabaena variabilis ATCC 29413; Arthrospira maxima CS-328; Arthrospira platensis str. paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Candidatus desulforudis audaxviator MP104C; Caldicellulosiruptor hydrothermalis_108; Clostridium phage c-st. Clostridium botulinum A3 str. Loch Maree; Clostridium botulinum Ba4 str. 657; Clostridium dificile QCD-63q42; Crocosphaera watsonii WH 8501; Cyanothece sp. ATCC 51142; Cyanothece sp. CCY0110; Cyanothece sp. PCC 7424; Cyanothece sp. PCC 7822 Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB 17; Methanohalobium evestigatum Z-7303; Microcystis phage Ma-LMMO1; Microcystis aeruginosa NIES-843; Aicroscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum SI; Petrotoga mobilis SJ95; Polaromonas naphthalenivorans CJ2; Polaromonas sp. JS666; Pseudoalteromonas haloplanktis TAC 125; Streptomyces pristinaespiralis ATCC 25486; Streptomyces pristnaespiralis ATCC 25486; Streptococcus thermophilus; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10(5): 726-737).

### Immunotherapy

In some embodiments, therapeutic applications utilizing SBPs may include immunotherapy. As used herein, the term “immunotherapy” refers to treatment of a disease, condition, or indication by modulating the immune system. Examples of immunotherapy approaches include the targeting of cancer antigens through monoclonal antibodies or through adoptive transfer of ex vivo engineered T cells (e.g., which contain chimeric antigen receptors or engineered T cell receptors). In some embodiments, SBPs may be used to modulate, alter, or exploit the immune system for the treatment of therapeutic indications. In some embodiments, SBPs may facilitate the delivery of material for treatment via immunotherapy. Examples of these materials include, but are not limited to, monoclonal antibodies, polyclonal antibodies, antigens, ex vivo engineered cells, interferons, interleukins, bacteria, microbiomes, microorganisms, colony-stimulating factors, and vaccines.

### Combinations

In some embodiments, SBPs may be administered in combination with other therapeutic agent and/or methods of treatment, e.g., with known pharmaceuticals and/or known therapeutic methods, such as, for example, those which are currently employed for treating these disorders. For example, SBPs used to treat cancer may be administered in combination with other anti-cancer treatments (e.g., biological, chemotherapy, or radiotherapy treatments).

### Diagnostics

In some embodiments, therapeutic applications utilizing SBPs may include diagnostic applications. In some embodiments, SBPs are used as diagnostic tools. In some embodiments. SBPs may be designed to undergo a detectable change in response to changes in the surrounding environment. Such SBPs may include any of those described in U.S. Pat. No. 9,802,374 or in Genovese et al. (2017) ACS Appl Mater Interfaces doi.10.1021acsami.7b13372, the contents of each of which are herein incorporated by reference in their entirety. Where detectable SBP changes correlate with environmental changes, SBP changes may be used to monitor the correlating environmental changes. Non-limiting examples of detectable SBP changes that may occur in response to environmental changes may include, but are not limited to, color, texture, elasticity, size, and attachment to other components. Non-limiting examples of environmental changes that may elicit changes in SBPs include, but are not limited to, the presence, absence, or levels of analytes (e.g., chemicals, metals, heavy metals, acids, bases, proteins, peptides, hormones, biomarkers, drugs, or small molecules), changes in acidity, changes in alkalinity, changes in redox state, changes in light, and changes in humidity.

In some embodiments, SBPs may be used as components of diagnostic devices. In some embodiments, a compound known to interact with an analyte (e.g., an antigen, binding partner, inhibitor, etc.) may be formulated as part of processed silk and incorporated into the device (e.g., as described in United States Publication Number US20170248593, the contents of which are herein incorporated by reference in their entirety). The introduction of an analyte may induce a color change indicative of the presence of that analyte. Diagnostic devices with components made or derived from SBPs of the present disclosure may enable the detection of a condition, disease, or indication (e.g., as described in United States Publication Number US20170248593, the contents of which are herein incorporated by reference in their entirety). Non-limiting examples of diseases that may be detected with a diagnostic device containing SBPs of the present disclosure include, but are not limited to, Ebola infection, HIV infection, and Lyme disease. Additional examples may include any of the therapeutic indications listed in Table 5, above.

### Tissue Engineering

In some embodiments, therapeutic applications utilizing SBPs may include tissue engineering. SBPs are attractive for tissue engineering due to their biocompatibility, bioavailability, low toxicity, non-inflammatory degradation products, and the ability to functionalize or formulate with other components needed for tissue culture. In some embodiments, SBPs are engineered tissues or are combined with engineered tissues. In some embodiments, SBPs are used for tissue engineering in vitro. In some embodiments, SBPs are used for tissue engineering in vivo. In some embodiments processed silks for tissue engineering are used to treat an indication in a subject. In some embodiments, processed silk is prepared and then applied to a tissue to treat the indication, as described in European Patent Number EP2276514, International Publication Number WO2017179069, Chantawong et al., and Du et al. (Chantawong et al. (2017) Mater Sci Mater Med 28(12):191; Du et al. (2017) Nanoscale Res Lett 12(1):573), the contents of each of which are herein incorporated by reference in their entirety. In some embodiments, processed silk is prepared, treated with tissue, and then utilized to treat the indication, as described in International Publication Number W2017137611, Zhou et al., Perteghella et al., and Weili et al. (Zhou et al. (2017) 1742-7061(17):30569; Perteghella et al. (2017) Macromol Biosci 17(9):1700131; Weili et al. (2017) Advanced Materials 29(29):e1701089), the contents of each of which are herein incorporated by reference in their entirety. Examples of tissues engineered with SBPs or processed silk scaffolds include, but are not limited to, bone tissue, cartilage and/or bone soft tissue, ear drum tissue, pancreatic tissue, skeletal muscle tissue, tympanic membrane tissue, bladder tissue, vascular tissue, nervous tissue, neural tissue, corneal tissue, spinal tissue, skin, and any other tissue relevant for the desired indication.

In some embodiments, engineered tissues may be used as model systems for additional study (e.g., as described in International Publication Number WO2017137937 or in Chen et al (2017) PloS One 12(11):e0187880, the contents of each of which are herein incorporated by reference in their entirety). In some embodiments, SBPs serve as a replacement for an existing tissue (e.g., as described in Chantawong et al (2017) Mater Sci Mater Med 28(12):191, the contents of which are herein incorporated by reference in their entirety). In some embodiments, SBPs serve as a scaffold for the growth of new tissues (e.g., as described in Ai et al. (2017) International Journal of Nanomedicine 12:7737-7750 or Chen et al. (2017) Stem Cell Research and Therapies 8:260, the contents of each of which are herein incorporated by reference in their entirety). In some embodiments. SBPs may be used as scaffolds for the growth of engineered tissue (e.g., as described in International Publication Number WO2017137937; Guo et al. (2017) Biomaterials 145:44-55; or Xiao et al. (2017) Oncotarget 8(49):86471-86487, the contents of each of which are herein incorporated by reference in their entirety).

In some embodiments, SBPs for tissue engineering are prepared with one or more other materials. These materials include, but are not limited to, any bioresorbable polymer matrix, albumin, alginate, bacterial cellulose, cellulose, cellulose acetate, any ceramic, chitin, chitosan, collagen, duck's feet collagen, elastin, fibrin, gelatin, glycerol, ionic liquids, magnesium oxide, melanin, any metal scaffold (e.g. cobalt-chromium-molybdenum composite), nano-hydroxyapatite, polyaniline, polycaprolactone, any polyethylene glycol, polyethylene glycol diglycidl ester, polyethylene oxide, polyurethane, quaternary ammonium chitosan, SBA 15, silica, any poly(α-ester) (e.g. polyglycolides, poly(lactide-co-glycolide), polyhydroxyalkanoates, any polycaprolactone, poly(propylene fumarate)), polyanhydrides, polyacetals, polyketals, polyorthoesters, polycarbonates, any polyurethane, polyphosphazenes, polyphosphoesters, any synthetic polyether, and any polysaccharide.

In some embodiments, tissue engineering with SBPs described herein may be used to repair existing tissue (e.g., as described in European Patent Numbers EP3215134 or EP3206725; or in Guo et al. (2017) Biomaterials 145:44-55; Chen et al. (2017) Stem Cell Research and Therapies 8:260; Xiao et al. (2017) Oncotarget 8(49):86471-86487; or Ruan et al. (2017) Biomed Pharmacother 97:600-606, the contents of each of which are herein incorporated by reference in their entirety). Examples of tissue repairs include, but are not limited to, bone repair, cartilage repair, bladder repair, organ repair, corneal repair, liver repair, muscle regeneration, vascular grafts, vascular patches, wound healing, and neuronal repair.

In some embodiments, SBPs used in tissue repair may be biodegradable or removable. Such SBPs may biodegrade or be removed after tissue repair and/or healing progresses or is completed. In some embodiments, SBPs may include or may be incorporated into devices used to stretch skin. Such devices may be used to prepare skin bubbles or flaps that can be used to cover or repair areas without skin or with skin damage. These devices may include balloons or other expandable materials that can be inflated or otherwise expanded over time. In some embodiments, SBPs are used to coat such devices to support biocompatibility.

In some embodiments, tissue engineering with SBPs described herein may be used to augment tissue (i.e., to add or expand tissue), as described in United States Publication Number US20170258573, European Patent Numbers EP2276514 or EP3206725 or in Yu et al. (2017) doi.10.1002/jbm.a.36297, the contents of each of which are herein incorporated by reference in their entirety. In some embodiments. SBPs may be used as implants or fillers to support tissue augmentation. In some embodiments, SBPs may be used in tissue augmentation related to or used for, implants, artificial organs, silk contact lenses, artificial blood vessels, stem cells, vascular patches, ear drum repair, tissue replacement, cartilage replacement, breast augmentation, surgical sutures, surgical meshes, wound dressing, bandages, and/or hemostatic sponges. In some embodiments, artificial organs may include artificial livers, as described in Janani et al. (2017) Acta Biomaterialia 157: 161-176, the contents of which are herein incorporated by reference in their entirety.

### Cell Culture

In some embodiments, therapeutic applications utilizing SBPs may include cell culture. In some embodiments, SBPs described herein may be used to facilitate cell culture in vitro, as described in Varone et al. (2017) Scientific Reports 7:13790, the contents of which are herein incorporated by reference in their entirety. In some embodiments, SBPs of the present disclosure may serve as a scaffold for in vitro cell culture, as described in Chen et al. (2017) Stem Cell and Res Therapy 8:260 or Chen et al. (2017) PloS One 12(1):e0187880, the contents of each of which are herein incorporated by reference in their entirety. These scaffolds may be a surface, structure, sponge, graft, mesh, gel, porous structure, or any other form conducive to cell culture known to those skilled in the art. In some embodiments, scaffolds are prepared with other components commonly used in cell culture (e.g., BSA, substance P, and culture media), as described in Chen et al. (2017) Stem Cell and Res Therapy 8:260 and Chen et al. (2017) PloS One 12(11):e0187880. In some embodiments, SBPs are optimized for cell adhesion, as described in Kambe et al. (2017) Materials (Basel) 10(10):e1153, the contents of which are herein incorporated by reference in their entirety. In some embodiments, cells cultured on SBPs may serve as models for further studies, as described in Chen et al. (2017) PloS One 12(11):e0187880. In some embodiments, the cells are cultured on a silk fibroin scaffold for the preparation of processed silk for subsequent use, as described in International Publication Number WO2017137611, United States Publication Number US20170312387, Li et al. (2017) Stem Cell Res Therapy 8(1):256, and Ciocci et al. (2017) Int J Biol Macromol S0141-8130(17):32839-8, the contents of each of which are herein incorporated by reference in their entirety. Subsequent uses of cells cultured using SBPs may include, but are not limited to, implants, patches, and scaffolds for tissue repair. Examples of cells that may be cultured on SBPs include, but are not limited to, human corneal stromal stem cells, human corneal epithelial cells, chicken dorsal root ganglions, bone mesenchymal stem cells, limbal epithelial stem cells, cardiac mesenchymal stem cells, adipose tissue-derived mesenchymal stem cells, periodontal ligament stem cells, human small intestinal enteroids, oral keratinocytes, fibroblasts, transfected fibroblasts, any 2-dimensional tissues, and any 3-dimensional tissues, T cells, embryonic stem cells, neural stem cells, mesenchymal stem cells. Chinese hamster ovary cells, insect cells, and hematopoietic stem cells.

### Preservative Applications

In some embodiments, SBPs may be used to preserve or stabilize therapeutic agents or other materials (e.g., agricultural compositions, agricultural products, materials, devices, and excipients). Such SBPs may be used to stabilize therapeutic agents used in therapeutic applications. In some embodiments, SBPs are used to maintain and/or improve the stability of therapeutic agents during lyophilization. The maintenance and/or improvement of stability during lyophilization may be determined by comparing products lyophilized with SBPs to products lyophilized with non-SBP formulation. Maintenance and/or improvement of stability during lyophilization will be found or appreciated by those of skill in the art when products lyophilized with SBPs are determined to impart superior or durational benefits over non-SBP formulations or those standard in the art.

In some embodiments, the SBPs maintain and/or improve therapeutic agent stability by at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 1 month, at least 6 weeks, at least 2 months, at least 10 weeks, at least 3 months, at least 14 weeks, at least 4 months, at least 18 weeks, at least 5 months, at least 22 weeks, at least 6 months, at least 7 months, at least 8 months, at least 9 months, at least 10 months, at least 11 months, at least a year, at least 2 years, at least 3 years, at least 4 years, at least 5 years, or more than 5 years.

Silk fibroin has been shown to stabilize compounds and prevent damage from heat over time, as described in Shimanovich et al (Shimanovich et al. (2015) Nature Communications 8:15902, the contents of which are herein incorporated by reference in their entirety). In some embodiments, a sensitive therapeutic agent may be loaded into an SBP, and the resulting compositions may protect that therapeutic agent from degradation and extend the time in which it could be active and functional. In some embodiments, the stabilization effects of SBPs may be combined with extended release effects. In some embodiments, a SBP may be created that releases a therapeutic agent over a long period of time, while maintaining peak efficacy of the molecule.

In some embodiments, SBPs may be used to stabilize cargo. Macromolecular therapeutic agents (e.g., large and/or bulky therapeutic agents and complexes), including proteins, antibodies, and/or biologics can aggregate and lose their function during manufacturing, storage, transportation, processing, and/or administration. Furthermore, a certain amount of a macromolecular therapeutic agent, such as proteins, can be lost due to adhesion to solid surfaces. The loss-due-to-adhesion problem is more impactful when the concentration of the macromolecular therapeutic agent is low. Because of their high molecular weight, macromolecular therapeutic agents are applied in lower concentrations compared to low molecular weight therapeutic agents, such as small molecules.

Currently, human serum albumin (HSA) is used to stabilize macromolecular agents used as therapeutics. Traditionally, stabilizing agents were selected based on lack of pharmacological activity and lack of immunological response. HSA is used as a stabilizer in various formulations as it inhibits nonspecific reactions that result in the denaturation of therapeutic agents. Furthermore, HSA can inhibit the macromolecules affinity to surfaces. While, the stabilizing agent should have no pharmacological activity, and should not stimulate an immunological response, because HSA is isolated from blood, it may be contaminated, for example with viruses, or contain an epitope that will generate an immunogenic response. In some embodiments, HSA may be replaced with SBPs to avoid the issues associated with HSA.

In some embodiments, SBPs may be used as a stabilizer for chemicals and therapeutic agents. Such uses may include those described for silk fibroin by Li et al. (Li et al. (2017) Biomacromolecules 19(9):2900-2905, the contents of which are herein incorporated by reference in their entirety). Silk fibroin protein has been used as a delivery vehicle for antibodies and is also known to be biodegradable and biocompatible. Hence, formulations using SBPs that include silk fibroin may provide improved properties as formulations for therapeutic agents and in particular larger therapeutic agents which tend to aggregate or lose efficacy when formulated at higher concentrations.

In some embodiments, SBPs may be used as a stabilizer for biological agents such as vaccines and antibiotics. Stability is a key factor to preserving potency and efficiency of sensitive biological agents, especially where the cold chain is unreliable. For vaccines, instability can cause loss of antigenicity and decreased infectivity. For antibiotics, this problem can lead to the development of antibiotic-resistant strains, a major public health concern. Factors affecting stability include temperature, light, humidity, and acidity or alkalinity of the agent (pH). Some agents may become unstable due to hydrolysis and aggregation of protein and carbohydrate molecules. SBPs of the present disclosure may be used to preserve the stability, or slow down the degradation process, of labile biological agents during storage and distribution. In some embodiments, SBPs of the present disclosure may be in combination with one or more of other stabilizers. Such stabilizers may include but are not limited to, MgCl₂, MgSO₄, monosodium glutamate (MSG), glycine, gelatin, 2-phenoxy-ethanol, lactose, sucrose, lactose-sorbitol, and sorbitol-gelatine, and human or bovine serum albumin.

### Surgical Applications

In some embodiments, therapeutic applications utilizing SBPs may include surgical applications. In some embodiments, SBPs may be incorporated into surgical tools, devices, and fabrics as described in Wang et al. (2017) J Biomed Mater Res A 106(1):221-230, the contents of which are herein incorporated by reference in their entirety. In some embodiments, SBPs may be used in surgical applications due to their antibiotic properties, e.g., as described in European Patent Number EP3226835 and in Mane et al. (2017) Scientific Reports 7:15531, the contents of each of which are herein incorporated by reference in their entirety. These antibiotic properties may be a general property of SBPs. The antibiotic properties of SBPs of the present disclosure may also be due to its payload. In some embodiments, SBPs of the present disclosure may be used for the delivery of therapeutics during and/or following surgery, e.g., as described in Sun et al. (Sun et al. (2017) Journal of Materials Chemistry B 5:8770-8779), the contents of which are herein incorporated by reference in their entirety. In some embodiments, SBPs may be used as bandages, patches, sponges, and/or sutures, e.g., as described in European Patent Number EP3215134, International Publication Number WO2001056626, and Seo et al. (Seo et al. (2017) J Biomater Appl 32(4):484-491), the contents of each of which are herein incorporated by reference in their entirety. In some embodiments, SBPs of the present disclosure may be used as a hemostatic agent to reduce bleeding and promote wound healing, e.g., as described in Seo et al. (Seo et al. (2017) J Biomater Appl 32(4):484-491), the contents of which are herein incorporated by reference in their entirety. In some embodiments, SBPs may be incorporated into surgical implants. e.g., as described in United States Publication Number US20170258573, the contents of which are herein incorporated by reference in their entirety. Examples of implants include, but are not limited to, breast implants, dental implants, bone implants, prostheses, buttock implants, cochlear implants, and implants for drug delivery.

In some embodiments, SBPs may be used in cosmetic surgery. Such SBPs may include prosthetics, implants, devices, sutures, or other components of cosmetic surgery known to those of skill in the art. In some embodiments, SBPs may be used in breast implants, e.g., as described in United States Publication Number US20170258573, the contents of which are herein incorporated by reference in their entirety.

In some embodiments, SBPs are used postoperatively to improve outcome, stabilize surgical sites, reduce inflammation, protect against infection, or reduce pain. Such SBPs may include one or more therapeutic agents (e.g., any of those described herein) as payloads.

In some embodiments, SBPs may be used in dental implants for drug delivery. A dental implant with a built-in reservoir allows the slow release of therapeutic agents, which could alleviate invasive procedure associated with chronic diseases. In some embodiments, such therapeutic agent delivered by a dental implant may include, but are not limited to, any of those listed in Table 3, above. As a non-limiting example, SBPs may be incorporated into dental implants for continuous release of insulin, as described in Li (2016) Int J Diabetes Clin Res, 3:057, the contents of which are herein incorporated by reference in their entirety. As a further example, SBPs may be used in dental implants for drug delivery against bacterial infection. Sharma et al. demonstrated that silk fibroin nanoparticles support in vitro sustained antibiotic release on titanium surface (Sharma et al. (2016) Nanomedicine. 12(5):1193-204, the contents of which are herein incorporated by reference in their entirety).

### Pharmaceutical Compositions

In some embodiments, SBPs are or are included in pharmaceutical compositions. As used herein, the term “pharmaceutical composition” refers to a composition designed and/or used for medicinal purposes (e.g. the treatment of a disease).

In some embodiments, pharmaceutical compositions include one or more excipients and/or one or more therapeutic agents. Excipients included in pharmaceutical compositions may include, but are not limited to, any of those listed in Table 1, above. Therapeutic agents included in pharmaceutical compositions may include, but are not limited to, any of those listed in Table 3, above. Relative amounts of therapeutic agents, excipient, and/or any additional ingredients in pharmaceutical compositions may vary, depending upon the identity, size, and/or condition of the subject being treated and further depending upon the route by which the composition is administered. For example, the composition may include from about 0.1% to about 99% (w/w) of a therapeutic agent.

Some excipients may include pharmaceutically acceptable excipients. The phrase “pharmaceutically acceptable” as used herein, refers to suitability within the scope of sound medical judgment for contacting subject (e.g., human or animal) tissues and/or bodily fluids with toxicity, irritation, allergic response, or other complication levels yielding reasonable benefit/risk ratios. As used herein, the term “pharmaceutically acceptable excipient” refers to any ingredient, other than active agents, that is substantially nontoxic and non-inflammatory in a subject. Pharmaceutically acceptable excipients may include, but are not limited to, solvents, dispersion media, diluents, inert diluents, buffering agents, lubricating agents, oils, liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of pharmaceutical compositions.

In some embodiments, SBP pharmaceutical compositions may include therapeutic nanoparticles. As used herein, the term “therapeutic nanoparticle” refers to nanoparticles that may be used to restore or promote the health and/or wellbeing of a subject and/or to treat, prevent, alleviate, cure, or diagnose a disease, disorder, or condition. In some embodiments, SBP therapeutic nanoparticles may be prepared and/or used according to any of the methods described in International Publication Numbers WO2010005740, WO2010030763, WO2010005721, WO2010005723, or WO2012054923; United States Publication. Numbers US20110262491, US20100104645, US20100087337, US20100068285, US20110274759, US20100068286 or US20120288541; or U.S. Pat. No. 8,206,747, 8,293,276, 8,318,208, or 8,318,211, the contents of each of which are herein incorporated by reference in their entirety.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of therapeutic agent or other compounds. The amount of therapeutic agent may generally be equal to the dosage of therapeutic agent administered to a subject and/or a convenient fraction of such dosage including, but not limited to, one-half or one-third of such a dosage.

In some embodiments, pharmaceutical compositions may include between 20 to 55% (w/w) silk fibroin. In some embodiments, the formulations of silk fibroin rods described herein may include between 40 to 80% (w/w) therapeutic agent. In some embodiments, pharmaceutical compositions may include about 33% (w/w) silk fibroin and about 67% (w/w) therapeutic agent. In some embodiments, pharmaceutical compositions may include about 25% (w/w) silk fibroin and about 75% (w/w) therapeutic agent. In some embodiments, pharmaceutical compositions may include about 20% (w/w) silk fibroin and about 80% (w/w) therapeutic agent. In some embodiments, pharmaceutical compositions may include about 40% (w/w) silk fibroin and about 60% (w/w) therapeutic agent. In some embodiments, pharmaceutical compositions may include about 29% (w/w) silk fibroin and about 71% (w/w) therapeutic agent. In some embodiments, pharmaceutical compositions may include about 40% (w/w) silk fibroin and about 60% (w/w) therapeutic agent.

In some embodiments, pharmaceutical compositions may include 35% (w/w) silk fibroin and 65% (w/w) therapeutic agent. In some embodiments, pharmaceutical compositions may include 30% (w/w) silk fibroin and 70% (w/w) therapeutic agent. In some embodiments, pharmaceutical compositions may include 40% (w/w) silk fibroin and 60% (w/w) therapeutic agent. In some embodiments, pharmaceutical compositions may include 26% (w/w) silk fibroin and 74% (w/w) therapeutic agent. In some embodiments, pharmaceutical compositions may include 37% (w/w) silk fibroin and 63% (w/w) therapeutic agent. In some embodiments, pharmaceutical compositions may include 33% (w/w) silk fibroin and 66% (w/v) therapeutic agent. In some embodiments, pharmaceutical compositions may include 51% (w/w) silk fibroin and 49% (w/w) therapeutic agent.

### Dosing

In some embodiments, the present disclosure provides methods of administering pharmaceutical compositions that are or include SBPs to subjects in need thereof. Such methods may include providing pharmaceutical compositions at one or more doses and/or according to a specific schedule. In some embodiments, doses may be determined based on desired amounts of therapeutic agent or SBP to be delivered. Doses may be adjusted to accommodate any route of administration effective for a particular therapeutic application. The exact amount required will vary from subject to subject, depending on the species, age, and general condition of the subject, the severity of the disease, the particular composition, its mode of administration, its mode of activity, and the like. The frequency of dosing required will also vary from subject to subject, depending on the species, age, and general condition of the subject, the severity of the disease, the particular composition, its mode of administration, its mode of activity, and the like.

SBPs may be formulated in dosage unit form. Such forms may allow for ease of administration and uniformity of dosage. Total daily SBP usage may be decided by an attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment drugs used in combination or coincidental with the specific compound employed; and like factors well known in the medical arts.

In some embodiments, pharmaceutical compositions that are or include SBPs may include a therapeutic agent or SBP at a concentration of from about 10 ng/mL to about 30 ng/mL, from about 12 ng/mL to about 32 ng/mL, from about 14 ng/mL to about 34 ng/mL, from about 16 ng/mL to about 36 ng/mL, from about 18 ng/mL to about 38 ng/mL, from about 20 ng/mL to about 40 ng/mL, from about 22 ng/mL to about 42 ng/mL, from about 24 ng/mL to about 44 ng/mL, from about 26 ng/mL to about 46 ng/mL, from about 28 ng/mL to about 48 ng/mL, from about 30 ng/mL to about 50 ng/mL, from about 35 ng/mL to about 55 ng/mL, from about 40 ng/mL to about 60 ng/mL, from about 45 ng/mL to about 65 ng/mL, from about 50 ng/mL to about 75 ng/mL, from about 60 ng/mL to about 240 ng/mL, from about 70 ng/mL to about 350 ng/mL, from about 80 ng/mL to about 400 ng/mL, from about 90 ng/mL to about 450 ng/mL, from about 100 ng/mL to about 500 ng/mL, from about 0.01 μg/mL to about 1 μg/mL, from about 0.05 μg/mL to about 2 μg/mL, from about 1 μg/mL to about 5 μg/mL, from about 2 μg/mL to about 10 μg/mL, from about 4 μg/mL to about 16 μg/mL, from about 5 μg/mL to about 20 μg/mL, from about 8 μg/mL to about 24 μg/mL, from about 10 μg/mL to about 30 μg/mL, from about 12 μg/mL to about 32 μg/mL, from about 14 μg/mL to about 34 μg/mL, from about 16 μg/mL to about 36 μg/mL, from about 18 μg/mL to about 38 μg/mL, from about 20 μg/mL to about 40 μg/mL, from about 22 μg/mL to about 42 μg/mL, from about 24 μg/mL to about 44 μg/mL, from about 26 μg/mL to about 46 μg/mL, from about 28 μg/mL to about 48 μg/mL, from about 30 μg/mL to about 50 μg/mL, from about 35 μg/mL to about 55 μg/mL, from about 40 μg/mL to about 60 μg/mL, from about 45 μg/mL to about 65 μg/mL, from about 50 μg/mL to about 75 μg/mL, from about 60 μg/mL to about 240 μg/mL, from about 70 μg/mL to about 350 μg/mL, from about 80 μg/mL to about 400 μg/mL, from about 90 μg/mL to about 450 μg/mL, from about 100 μg/mL to about 500 μg/mL, from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 36 mg/mL, from about 18 mg/mL to about 38 mg/mL, from about 20 mg/mL to about 40 mg/mL, from about 22 mg/mL to about 42 mg/mL, from about 24 mg/mL to about 44 mg/mL, from about 26 mg/mL to about 46 mg/mL, from about 28 mg/mL to about 48 mg/mL, from about 30 mg/mL to about 50 mg/mL, from about 40 mg/mL to about 100 mg/mL, or more than 100 mg/mL.

In some embodiments, pharmaceutical compositions that are or include SBPs may be administered at a dose that provides subjects with a mass of therapeutic agent or SBP per unit mass of the subject (e.g., mg therapeutic agent or SBP per kg of subject [mg/kg]). In some embodiments, therapeutic agents or SBPs are administered at a dose of from about 1 ng/kg to about 5 ng/kg, from about 2 ng/kg to about 10 ng/kg, from about 4 ng/kg to about 16 ng/kg, from about 5 ng/kg to about 20 ng/kg, from about 8 ng/kg to about 24 ng/kg, from about 10 ng/kg to about 30 ng/kg, from about 12 ng/kg to about 32 ng/kg, from about 14 ng/kg to about 34 ng/kg, from about 16 ng/kg to about 36 ng/kg, from about 18 ng/kg to about 38 ng/kg, from about 20 ng/kg to about 40 ng/kg, from about 22 ng/kg to about 42 ng/kg, from about 24 ng/kg to about 44 ng/kg, from about 26 ng/kg to about 46 ng/kg, from about 28 ng/kg to about 48 ng/kg, from about 30 ng/kg to about 50 ng/kg, from about 35 ng/kg to about 55 ng/kg, from about 40 ng/kg to about 60 ng/kg, from about 45 ng/kg to about 65 ng/kg, from about 50 ng/kg to about 75 ng/kg, from about 60 ng/kg to about 240 ng/kg, from about 70 ng/kg to about 350 ng/kg, from about 80 ng/kg to about 400 ng/kg, from about 90 ng/kg to about 450 ng/kg, from about 100 ng/kg to about 500 ng/kg, from about 0.01 μg/kg to about 1 μg/kg, from about 0.05 μg/kg to about 2 μg/kg, from about 1 μg/kg to about 5 μg/kg, from about 2 μg/kg to about 10 μg/kg, from about 4 μg/kg to about 16 μg/kg, from about 5 μg/kg to about 20 μg/kg, from about 8 μg/kg to about 24 μg/kg, from about 10 μg/kg to about 30 μg/kg, from about 12 μg/kg to about 32 μg/kg, from about 14 μg/kg to about 34 μg/kg, from about 16 μg/kg to about 36 μg/kg, from about 18 μg/kg to about 38 μg/kg, from about 20 μg/kg to about 40 μg/kg, from about 22 μg/kg to about 42 μg/kg, from about 24 μg/kg to about 44 μg/kg, from about 26 μg/kg to about 46 μg/kg, from about 28 μg/kg to about 48 μg/kg, from about 30 μg/kg to about 50 μg/kg, from about 35 μg/kg to about 55 μg/kg, from about 40 μg/kg to about 60 μg/kg, from about 45 μg/kg to about 65 μg/kg, from about 50 μg/kg to about 75 μg/kg, from about 60 μg/kg to about 240 μg/kg, from about 70 μg/kg to about 350 μg/kg, from about 80 μg/kg to about 400 μg/kg, from about 90 μg/kg to about 450 μg/kg, from about 100 μg/kg to about 500 μg/kg, from about 0.01 mg/kg to about 1 mg/kg, from about 0.05 mg/kg to about 2 mg/kg, from about 1 mg/kg to about 5 mg/kg, from about 2 mg/kg to about 10 mg/kg, from about 4 mg/kg to about 16 mg/kg, from about 5 mg/kg to about 20 mg/kg, from about 8 mg/kg to about 24 mg/kg, from about 10 mg/kg to about 30 mg/kg, from about 12 mg/kg to about 32 mg/kg, from about 14 mg/kg to about 34 mg/kg, from about 16 mg/kg to about 36 mg/kg, from about 18 mg/kg to about 38 mg/kg, from about 20 mg/kg to about 40 mg/kg, from about 22 mg/kg to about 42 mg/kg, from about 24 mg/kg to about 44 mg/kg, from about 26 mg/kg to about 46 mg/kg, from about 28 mg/kg to about 48 mg/kg, from about 30 mg/kg to about 50 mg/kg, from about 35 mg/kg to about 55 mg/kg, from about 40 mg/kg to about 60 mg/kg, from about 45 mg/kg to about 65 mg/kg, from about 50 mg/kg to about 75 mg/kg, from about 60 mg/kg to about 240 mg/kg, from about 70 mg/kg to about 350 mg/kg, from about 80 mg/kg to about 400 mg/kg, from about 90 mg/kg to about 450 mg/kg, from about 100 mg/kg to about 500 mg/kg, from about 0.01 g/kg to about 1 g/kg, from about 0.05 g/kg to about 2 g/kg, from about 1 g/kg to about 5 g/kg, or more than 5 g/kg.

In some embodiments, pharmaceutical compositions that are or include SBPs may be administered at a dose sufficient to yield desired therapeutic agent or SBP concentration levels in subject tissue or fluids (e.g., blood, plasma, urine, etc.). In some embodiments, doses are adjusted to achieve subject therapeutic agent or SBP concentration levels in subject tissues or fluids of from about 1 pg/mL to about 5 pg/mL, from about 2 pg/mL to about 10 pg/mL, from about 4 pg/mL to about 16 pg/mL, from about 5 pg/mL to about 20 pg/mL, from about 8 pg/mL to about 24 pg/mL, from about 10 pg/mL to about 30 pg/mL, from about 12 pg/mL to about 32 pg/mL, from about 14 pg/mL to about 34 pg/mL, from about 16 pg/mL to about 36 pg/mL, from about 18 pg/mL to about 38 pg/mL, from about 20 pg/mL to about 40 pg/mL, from about 22 pg/mL to about 42 pg/mL, from about 24 pg/mL to about 44 pg/mL, from about 26 pg/mL to about 46 pg/mL, from about 28 pg/mL to about 48 pg/mL, from about 30 pg/mL to about 50 pg/mL, from about 35 pg/mL to about 55 pg/mL, from about 40 pg/mL to about 60 pg/mL, from about 45 pg/mL to about 65 pg/mL, from about 50 pg/mL to about 75 pg/mL, from about 60 pg/mL to about 240 pg/mL, from about 70 pg/mL to about 350 pg/mL, from about 80 pg/mL to about 400 pg/mL, from about 90 pg/mL to about 450 pg/mL, from about 100 pg/mL to about 500 pg/mL, from about 0.01 ng/mL to about 1 ng/mL, from about 0.05 ng/mL to about 2 ng/mL, from about 1 ng/mL to about 5 ng/mL, from about 2 ng/mL to about 10 ng/mL, from about 4 ng/mL to about 16 ng/mL, from about 5 ng/mL to about 20 ng/mL, from about 8 ng/mL to about 24 ng/mL, from about 10 ng/mL to about 30 ng/mL, from about 12 ng/mL to about 32 ng/mL, from about 14 ng/mL to about 34 ng/mL, from about 16 ng/mL to about 36 ng/mL, from about 18 ng/mL to about 38 ng/mL, from about 20 ng/mL to about 40 ng/mL, from about 22 ng/mL to about 42 ng/mL, from about 24 ng/mL to about 44 ng/mL, from about 26 ng/mL to about 46 ng/mL, from about 28 ng/mL to about 48 ng/mL, from about 30 ng/mL to about 50 ng/mL, from about 35 ng/mL to about 55 ng/mL, from about 40 ng/mL to about 60 ng/mL, from about 45 ng/mL to about 65 ng/mL, from about 50 ng/mL to about 75 ng/mL, from about 60 ng/mL to about 240 ng/mL, from about 70 ng/mL to about 350 ng/mL, from about 80 ng/mL to about 400 ng/mL, from about 90 ng/mL to about 450 ng/mL, from about 100 ng/mL to about 500 ng/mL, from about 0.01 μg/mL to about 1 μg/mL, from about 0.05 μg/mL to about 2 μg/mL, from about 1 μg/mL to about 5 μg/mL, from about 2 μg/mL to about 10 μg/mL, from about 4 μg/mL to about 16 μg/mL, from about 5 μg/mL to about 20 μg/mL, from about 8 μg/mL to about 24 μg/mL, from about 10 μg/mL to about 30 μg/mL, from about 12 μg/mL to about 32 μg/mL, from about 14 μg/mL to about 34 μg/mL, from about 16 μg/mL to about 36 μg/mL, from about 18 μg/mL to about 38 μg/mL, from about 20 μg/mL to about 40 μg/mL, from about 22 μg/mL to about 42 μg/mL, from about 24 μg/mL to about 44 μg/mL, from about 26 μg/mL to about 46 μg/mL, from about 28 μg/mL to about 48 μg/mL, from about 30 μg/mL to about 50 μg/mL, from about 35 μg/mL to about 55 μg/mL, from about 40 μg/mL to about 60 μg/mL, from about 45 μg/mL to about 65 μg/mL, from about 50 μg/mL to about 75 μg/mL, from about 60 μg/mL to about 240 μg/mL, from about 70 μg/mL to about 350 μg/mL, from about 80 μg/mL to about 400 μg/mL, from about 90 μg/mL to about 450 μg/mL, from about 100 μg/mL to about 500 μg/mL, from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 36 mg/mL, from about 18 mg/mL to about 38 mg/mL, from about 20 mg/mL to about 40 mg/mL, from about 22 mg/mL to about 42 mg/mL, from about 24 mg/mL to about 44 mg/mL, from about 26 mg/mL to about 46 mg/mL, from about 28 mg/mL to about 48 mg/mL, from about 30 mg/mL to about 50 mg/mL, from about 35 mg/mL to about 55 mg/mL, from about 40 mg/mL to about 60 mg/mL, from about 45 mg/mL to about 65 mg/mL, from about 50 mg/mL to about 75 mg/mL, from about 60 mg/mL to about 240 mg/mL, from about 70 mg/mL to about 350 mg/mL, from about 80 mg/mL to about 400 mg/mL, from about 90 mg/mL to about 450 mg/mL, from about 100 mg/mL to about 500 mg/mL, from about 0.01 g/mL to about 1 g/mL.

In some embodiments, pharmaceutical compositions that are or include SBPs are provided in one or more doses and are administered one or more times to subjects. Some pharmaceutical compositions are provided in only a single administration. Some pharmaceutical compositions are provided according to a dosing schedule that include two or more administrations. Each administration may be at the same dose or may be different from a previous and/or subsequent dose. In some embodiments, subjects are provided an initial dose that is higher than subsequent doses (referred to herein as a “loading dose”). In some embodiments, doses are decreased over the course of administration. In some embodiments, dosing schedules include pharmaceutical composition administration from about every 2 hours to about every 10 hours, from about every 4 hours to about every 20 hours, from about every 6 hours to about every 30 hours, from about every 8 hours to about every 40 hours, from about every 10 hours to about every 50 hours, from about every 12 hours to about every 60 hours, from about every 14 hours to about every 70 hours, from about every 16 hours to about every 80 hours, from about every 18 hours to about every 90 hours, from about every 20 hours to about every 100 hours, from about every 22 hours to about every 120 hours, from about every 24 hours to about every 132 hours, from about every 30 hours to about every 144 hours, from about every 36 hours to about every 156 hours, from about every 48 hours to about every 168 hours, from about every 2 days to about every 10 days, from about every 4 days to about every 15 days, from about every 6 days to about every 20 days, from about every 8 days to about every 25 days, from about every 10 days to about every 30 days, from about every 12 days to about every 35 days, from about every 14 days to about every 40 days, from about every 16 days to about every 45 days, from about every 18 days to about every 50 days, from about every 20 days to about every 55 days, from about every 22 days to about every 60 days, from about every 24 days to about every 65 days, from about every 30 days to about every 70 days, from about every 2 weeks to about every 8 weeks, from about every 3 weeks to about every 12 weeks, from about every 4 weeks to about every 16 weeks, from about every 5 weeks to about every 20 weeks, from about every 6 weeks to about every 24 weeks, from about every 7 weeks to about every 28 weeks, from about every 8 weeks to about every 32 weeks, from about every 9 weeks to about every 36 weeks, from about every 10 weeks to about every 40 weeks, from about every 11 weeks to about every 44 weeks, from about every 12 weeks to about every 48 weeks, from about every 14 weeks to about every 52 weeks, from about every 16 weeks to about every 56 weeks, from about every 20 weeks to about every 60 weeks, from about every 2 months to about every 6 months, from about every 3 months to about every 12 months, from about every 4 months to about every 18 months, from about every 5 months to about every 24 months, from about every 6 months to about every 30 months, from about every 7 months to about every 36 months, from about every 8 months to about every 42 months, from about every 9 months to about every 48 months, from about every 10 months to about every 54 months, from about every 11 months to about every 60 months, from about every 12 months to about every 66 months, from about 2 years to about 5 years, from about 3 years to about 10 years, from about 4 years to about 15 years, from about 5 years to about 20 years, from about 6 years to about 25 years, from about 7 years to about 30 years, from about 8 years to about 35 years, from about 9 years to about 40 years, from about 10 years to about 45 years, from about 15 years to about 50 years, or more than every 50 years.

In some embodiments, pharmaceutical compositions that are or include SBPs may be administered at a dose sufficient to provide a therapeutically effective amount of therapeutic agents or SBPs. As used herein, the term “therapeutically effective amount” refers to an amount of an agent sufficient to achieve a therapeutically effective outcome. As used herein, the term “therapeutically effective outcome” refers to a result of treatment where at least one objective of treatment is met. In some embodiments, a therapeutically effective amount is provided in a single dose. In some embodiments, a therapeutically effective amount is administered according to a dosing schedule that includes a plurality of doses. Those skilled in the art will appreciate that in some embodiments, a unit dosage form may be considered to include a therapeutically effective amount of a particular agent or entity if it includes an amount that is effective when administered as part of such a dosage regimen.

### Administration

In some embodiments, pharmaceutical compositions that are or include SBPs may be administered according to one or more administration routes. In some embodiments, administration is enteral (into the intestine), transdermal, intravenous bolus, intralesional (within or introduced directly to a localized lesion), intrapulmonary (within the lungs or its bronchi), diagnostic, intraocular (within the eye), transtympanic (across or through the tympanic cavity), intravesical infusion, sublingual, nasogastric (through the nose and into the stomach), spinal, intracartilaginous (within a cartilage), insufflation (snorting), rectal, intravascular (within a vessel or vessels), buccal (directed toward the cheek), dental (to a tooth or teeth), intratesticular (within the testicle), intratympanic (within the aurus media), percutaneous, intrathoracic (within the thorax), submucosal, cutaneous, epicutaneous (application onto the skin), dental intracomal, intramedullary (within the marrow cavity of a bone), intra-abdominal, epidural (into the dura matter), intramuscular (into a muscle), intralymphatic (within the lymph), iontophoresis (by means of electric current where ions of soluble salts migrate into the tissues of the body), subcutaneous (under the skin), intragastric (within the stomach), nasal administration (through the nose), transvaginal, intravenous drip, endosinusial, intraprostatic (within the prostate gland), soft tissue, intradural (within or beneath the dura), subconjunctival, oral (by way of the mouth), peridural, parenteral, intraduodenal (within the duodenum), intracisternal (within the cistema magna cerebellomedularis), periodontal, periarticular, biliary perfusion, intracoronary (within the coronary arteries), intrathecal (within the cerebrospinal fluid at any level of the cerebrospinal axis), intrameningeal (within the meninges), intracavernous injection (into a pathologic cavity) intracavitary (into the base of the penis), intrabiliary, subarachnoid, intrabursal, ureteral (to the ureter), intratendinous (within a tendon), auricular (in or by way of the ear), intracardiac (into the heart), enema, intraepidermal (to the epidermis), intraventricular (within a ventricle), intramyocardial (within the myocardium), intratubular (within the tubules of an organ), vaginal, sublabial, intracorporus cavernosum (within the dilatable spaces of the corporus cavernosa of the penis), intradermal (into the skin itself), intravitreal (through the eye), perineural, cardiac perfusion, irrigation (to bathe or flush open wounds or body cavities), in ear drops, endotracheal, intraosseous infusion (into the bone marrow), caudal block, intrauterine, transtracheal (through the wall of the trachea), intra-articular, intracorneal (within the cornea), endocervical, extracorporeal, intraspinal (within the vertebral column), transmucosal (diffusion through a mucous membrane), topical, photopheresis, oropharyngeal (directly to the mouth and pharynx), occlusive dressing technique (topical route administration which is then covered by a dressing which occludes the area), transplacental (through or across the placenta), intrapericardial (within the pericardium), intraarterial (into an artery), interstitial, intracerebral (into the cerebrum), intracerebroventricular (into the cerebral ventricles), peridural, intrapleural (within the pleura), infiltration, intrabronchial, intrasinal (within the nasal or periorbital sinuses), intraductal (within a duct of a gland), transdermal (diffusion through the intact skin for systemic distribution), intracaudal (within the cauda equine), nerve block, retrobulbar (behind the pons or behind the eyeball), intravenous (into a vein), intra-amniotic, conjunctival, intrasynovial (within the synovial cavity of a joint), gastroenteral, intraluminal (within a lumen of a tube), intrathecal (into the spinal canal), electro-osmosis, intraileal (within the distal portion of the small intestine), intraesophageal (to the esophagus), extra-amniotic administration, hemodialysis, intragingival (within the gingivae), intratumor (within a tumor), eye drops (onto the conjunctiva), laryngeal (directly upon the larynx), urethral (to the urethra), intravaginal administration, intramyocardial (entering the myocardium), intraperitoneal (infusion or injection into the peritoneum), respiratory (within the respiratory tract by inhaling orally or nasally for local or systemic effect), intradiscal (within a disc), ophthalmic (to the external eye), and/or intraovarian (within the ovary).

In some embodiments, pharmaceutical compositions that are or include SBPs may be administered by auricular administration, intraarticular administration, intramuscular administration, intrathecal administration, extracorporeal administration, buccal administration, intrabronchial administration, conjunctival administration, cutaneous administration, dental administration, endocervical administration, endosinusial administration, endotracheal administration, enteral administration, epidural administration, intra-abdominal administration, intrabiliary administration, intrabursal administration, oropharyngeal administration, interstitial administration, intracardiac administration, intracartilaginous administration, intracaudal administration, intracavernous administration, intracerebral administration, intracorporous cavernosum, intracavitary administration, intracorneal administration, intracisternal administration, cranial administration, intracranial administration, intradermal administration, intralesional administration, intratympanic administration, intragingival administration, intraovarian administration, intraocular administration, intradiscal administration, intraductal administration, intraduodenal administration, ophthalmic administration, intradural administration, intraepidermal administration, intraesophageal administration, nasogastric administration, nasal administration, laryngeal administration, intraventricular administration, intragastric administration, intrahepatic administration, intaluminal administration, intravitreal administration, intravesicular administration, intralymphatic administration, intramammary administration, intramedullary administration, intrasinal administration, intrameningeal administration, intranodal administration, intraovarian administration, intrapulmonary administration, intrapericardial administration, intraperitoneal administration, intrapleural administration, intrapericardial administration, intraprostatic administration, intrapulmonary administration, intraluminal administration, intraspinal administration, intrasynovial administration, intratendinous administration, intratesticular administration, subconjunctival administration, intracerbroventricular administration, epicutaneous administration, intravenous administration, retrobulbar administration, periarticular administration, intrathoracic administration, subarachnoid administration, intratubular administration, periodontal administration, transtympanic administration, transtracheal administration, intratumor administration, vaginal administration, urethral administration, intrauterine administration, oral administration, gastroenteral administration, parenteral administration, sublingual administration, ureteral administration, percutaneous administration, peridural administration, transmucosal administration, perineural administration, transdermal administration, rectal administration, soft tissue administration, intraarterial administration, subcutaneous administration, topical administration, extra-amniotic administration, insufflation, enema, eye drops, ear drops, or intravesical infusion.

In some embodiments, the SBPs described herein may be administered via injection. Injection site reactions may be monitored via any method known to one skilled in the art. In some embodiments, SBPs described herein may be administered via intravitreal injection. In some embodiments, SBPs described herein may be administered using any form of injection device, for example a syringe/needle device of a gauge suitable for the application. In some embodiments the administration is intravitreal using a 22-gauge needle. In some embodiments, the administration is intravitreal using a 27-gauge needle.

In some embodiments, SBPs may be administered for localized treatment (e.g., see United States Publication Numbers US20170368236 and US20110171239, the contents of each of which are herein incorporated by reference in their entirety). In some embodiments, SBPs may be administered for treatment of areas located further away from administration sites (e.g., see Aykac et al. (2017) Gene s0378-1119(17)30868-30865, the contents of which are herein incorporated by reference in their entirety).

In some embodiments, the SBPs are administered topically. In some embodiments, the SBP is in any format (e.g. solution or hydrogel) described in the present disclosure. In some embodiments, the SBP is a solution. In some embodiments, the SBP is a hydrogel. As a non-limiting example, the SBP is in the form of a hydrogel and the route of delivery is topical.

In some embodiments, SBP administration or SBP-based therapeutic agent administration occurs over a period of time, referred to herein as the “administration period.” During administration periods, administration may be continuous or may be separated into two or more administrations. In some embodiments, administration periods may be from about 1 min to about 30 min, from about 10 min to about 45 min, from about 20 min to about 60 min, from about 40 min to about 90 min, from about 2 hours to about 10 hours, from about 4 hours to about 20 hours, from about 6 hours to about 30 hours, from about 8 hours to about 40 hours, from about 10 hours to about 50 hours, from about 12 hours to about 60 hours, from about 14 hours to about 70 hours, from about 16 hours to about 80 hours, from about 18 hours to about 90 hours, from about 20 hours to about 100 hours, from about 22 hours to about 120 hours, from about 24 hours to about 132 hours, from about 30 hours to about 144 hours, from about 36 hours to about 156 hours, from about 48 hours to about 168 hours, from about 2 days to about 10 days, from about 4 days to about 15 days, from about 6 days to about 20 days, from about 8 days to about 25 days, from about 10 days to about 30 days, from about 12 days to about 35 days, from about 14 days to about 40 days, from about 16 days to about 45 days, from about 18 days to about 50 days, from about 20 days to about 55 days, from about 22 days to about 60 days, from about 24 days to about 65 days, from about 30 days to about 70 days, from about 2 weeks to about 8 weeks, from about 3 weeks to about 12 weeks, from about 4 weeks to about 16 weeks, from about 5 weeks to about 20 weeks, from about 6 weeks to about 24 weeks, from about 7 weeks to about 28 weeks, from about 8 weeks to about 32 weeks, from about 9 weeks to about 36 weeks, from about 10 weeks to about 40 weeks, from about 11 weeks to about 44 weeks, from about 12 weeks to about 48 weeks, from about 14 weeks to about 52 weeks, from about 16 weeks to about 56 weeks, from about 20 weeks to about 60 weeks, from about 2 months to about 6 months, from about 3 months to about 12 months, from about 4 months to about 18 months, from about 5 months to about 24 months, from about 6 months to about 30 months, from about 7 months to about 36 months, from about 8 months to about 42 months, from about 9 months to about 48 months, from about 10 months to about 54 months, from about 11 months to about 60 months, from about 12 months to about 66 months, from about 2 years to about 5 years, from about 3 years to about 10 years, from about 4 years to about 15 years, from about 5 years to about 20 years, from about 6 years to about 25 years, from about 7 years to about 30 years, from about 8 years to about 35 years, from about 9 years to about 40 years, from about 10 years to about 45 years, from about 15 years to about 50 years, or more than 50 years.

### Depot Administration

In some embodiments, SBPs may be administered by or be used to administer therapeutic agents by depot administration. As used herein, the term “depot” refers to a concentration of one or more agents in a particular region or in association with a composition or device. With depot administration, the one or more agents exit or diffuse from the concentration into surrounding areas. Agents administered by depot administration may be SBPs. In some embodiments, SBPs are depots for therapeutic agents, wherein the therapeutic agents exit or diffuse from the SBPs. In some embodiments, the SBPs may be utilized for the local delivery of therapeutic agents. In some embodiments, depots are implants. In some embodiments, depots are gels or hydrogels. In some embodiments, depot administration of an SBP may reduce the number of times a therapeutic agent needs to be administered. In some embodiments, depot administration of an SBP may replace oral administration of a therapeutic agent.

### Controlled Release

In some embodiments, SBPs and related methods described herein be may be used for controlled release of therapeutic agents. As used herein, the term “controlled release” refers to regulated movement of factors from specific locations to surrounding areas. In some embodiments, the specific location is a depot. Controlled release of factors from depots may be regulated by interactions between therapeutic agents and depot components. Such interactions may, for example, modulate therapeutic agent diffusion rate and/or affect therapeutic agent stability and/or degradation. In some embodiments, the depot is an SBP. In some embodiments, factors subject to controlled release from depots are SBPs. In some embodiments, therapeutic agents are subject to controlled release from SBP depots.

In some embodiments, SBPs may control payload release by extending payload half-life. As used herein, the term “half-life” refers to the length of time necessary for levels of a factor to be reduced (e.g., through clearance or degradation) by 50%. Some payloads may exhibit shortened half-life in water (e.g., due to hydrolysis). SBPs may protect payloads from exposure to water, thereby improving payload half-life. In other cases, SBPs may protect payloads from exposure to acidic conditions (e.g., gastric pH) and maintain encapsulation/stabilization of the payloads. In some embodiments, methods of increasing payload half-life using SBPs may include any of those described in United States Publication US20100028451, the contents of which are herein incorporated by reference in their entirety. Methods of improving payload half-life may be carried out in vitro or in vivo. In some embodiments, SBP-based methods of improving payload half-life may enable therapeutic indication treatment with fewer doses and/or treatments. Such methods may include any of those described in International Publication Number WO2017139684, the contents of which are herein incorporated by reference in their entirety. In some embodiments, payload half-life may be extended by from about 0.01% to about 1%, from about 0.05% to about 2%, from about 1% to about 5%, from about 2% to about 10%, from about 3% to about 15%, from about 4% to about 20%, from about 5% to about 25%, from about 6% to about 30%, from about 7% to about 35%, from about 8% to about 40%, from about 9% to about 45%, from about 10% to about 50%, from about 12% to about 55%, from about 14% to about 60%, from about 16% to about 65%, from about 18% to about 70%, from about 20% to about 75%, from about 22% to about 80%, from about 24% to about 85%, from about 26% to about 90%, from about 28% to about 95%, from about 30% to about 100%, from about 32% to about 105%, from about 34% to about 110%, from about 36% to about 115%, from about 38% to about 120%, from about 40% to about 125%, from about 42% to about 130%, from about 44% to about 135%, from about 46% to about 140%, from about 48% to about 145% from about 50% to about 150%, from about 60% to about 175%, from about 70% to about 200%, from about 80% to about 225%, from about 90% to about 250%, from about 100% to about 275%, from about 110% to about 300%, from about 120% to about 325%, from about 130% to about 350%, from about 140% to about 375%, from about 150% to about 400%, from about 170% to about 450%, from about 190% to about 500%, from about 210% to about 550%, from about 230% to about 600%, from about 250% to about 650%, from about 270% to about 700%, from about 290% to about 750%, from about 310% to about 800%, from about 330% to about 850%, from about 350% to about 900%, from about 370% to about 950%, from about 390% to about 1000%, from about 410% to about 1050%, from about 430% to about 1100%, from about 450% to about 1500%, from about 480% to about 2000%, from about 510% to about 2500%, from about 540% to about 3000%, from about 570% to about 3500%, from about 600% to about 4000%, from about 630% to about 4500%, from about 660% to about 5000%, from about 690% to about 5500%, from about 720% to about 6000%, from about 750% to about 6500%, from about 780% to about 7000%, from about 810% to about 7500%, from about 840% to about 8000%, from about 870% to about 8500%, from about 900% to about 9000%, from about 930% to about 9500%, from about 960% to about 10000%, or more than 10000%.

In some embodiments, SBP depots may be used for controlled release of therapeutic agents, wherein release is facilitated by diffusion. Such methods may include any of those described in United States Publication Number US20170333351, the contents of which are herein incorporated by reference in their entirety. Therapeutic agent diffusion may be slowed (i.e., controlled) by SBP depots leading to extended release periods. Extended therapeutic agent release periods may enable longer administration periods. In some embodiments, administration periods are extended by from about 0.01% to about 1%, from about 0.05% to about 2%, from about 1% to about 5%, from about 2% to about 10%, from about 3% to about 15%, from about 4% to about 20%, from about 5% to about 25%, from about 6% to about 30%, from about 7% to about 35%, from about 8% to about 40%, from about 9% to about 45%, from about 10% to about 50%, from about 12% to about 55%, from about 14% to about 60%, from about 16% to about 65%, from about 18% to about 70%, from about 20% to about 75%, from about 22% to about 80%, from about 24% to about 85%, from about 26% to about 90%, from about 28% to about 95%, from about 30% to about 100%, from about 32% to about 105%, from about 34% to about 110%, from about 36% to about 115%, from about 38% to about 120%, from about 40% to about 125%, from about 42% to about 130%, from about 44% to about 135% from about 46% to about 140%, from about 48% to about 145%, from about 50% to about 150%, from about 60% to about 175%, from about 70% to about 200%, from about 80% to about 225%, from about 90% to about 250%, from about 100% to about 275%, from about 110% to about 300%, from about 120% to about 325%, from about 130% to about 350%, from about 140% to about 375%, from about 150% to about 400%, from about 170% to about 450%, from about 190% to about 500%, from about 210% to about 550%, from about 230% to about 600%, from about 250% to about 650%, from about 270% to about 700%, from about 290% to about 750%, from about 310% to about 800%, from about 330% to about 850%, from about 350% to about 900%, from about 370% to about 950%, from about 390% to about 1000%, from about 410% to about 1050%, from about 430% to about 1100%, from about 450% to about 1500%, from about 480% to about 2000%, from about 510% to about 2500%, from about 540% to about 3000%, from about 570% to about 3500%, from about 600% to about 4000%, from about 630% to about 4500%, from about 660% to about 5000%, from about 690% to about 5500%, from about 720% to about 6000%, from about 750% to about 6500%, from about 780% to about 7000%, from about 810% to about 7500%, from about 840% to about 8000%, from about 870% to about 8500%, from about 900% to about 9000%, from about 930% to about 9500%, from about 960% to about 10000%.

In some embodiments, the controlled release of a therapeutic agent for the treatment of a condition, disease, or indication may be facilitated by the degradation and/or dissolution of SBPs. Such methods may be carried according to those described in International Publication Numbers WO2013126799, WO2017165922, and U.S. Pat. No. 8,530,625, the contents of each of which are herein incorporated by reference in their entirety. SBP degradation and/or dissolution may expose increasing amounts of therapeutic agents over time for treatment of therapeutic indications.

In some embodiments, therapeutic agent release from SBPs may be monitored via high performance liquid chromatography (HPLC), ultra-performance liquid chromatography (UPLC), and/or other methods known to those skilled in the art.

SBP hydrogels may be used to extend payload release periods (e.g., as shown for extended release of small molecule in International Publication Number WO2017139684, the contents of which are herein incorporated by reference in their entirety. In some embodiments, SBP hydrogels are used to provide extended release of therapeutic agents (e.g., biological agents). Hydrogel networks may stabilize such agents and support their release as the hydrogel degrades. This effect serves to extend agent release and may be modulated by varying factors including processed silk molecular weight, concentration, excipient type, pH, and temperature. In some embodiments, processed silk molecular weight, concentration, excipient type, pH, and processing temperature used to prepare SBPs may be modulated to achieve desired payload release periods for specific therapeutic agents.

In some embodiments, SBPs may be lyophilized together with therapeutic agents. In some embodiments, combined lyophilization may induce further interactions between therapeutic agents and SBPs. These interactions may be maintained through SBP preparation and support extended payload release. Payload release may be dependent on SBP degradation and/or dissolution. In some embodiments, SBP β-sheet content is increased (e.g., via water annealing), thereby increasing SBP insolubility in water. Such SBPs may exhibit increased payload release periods. In some embodiments, these SBPs may include therapeutic agent stabilizing properties to extend administration periods and/or therapeutic agent half-life.

In some embodiments, SBPs described herein maintain and/or improve the controlled delivery of a therapeutic agent. In some embodiments, SBPs lengthen payload release period and/or administration period by at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some embodiments, SBPs lengthen payload release period and/or administration period by at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 1 month, at least 6 weeks, at least 2 months, at least 10 weeks, at least 3 months, at least 6 months, at least 9 months, or at least 1 year.

In some embodiments, SBPs may be used to modulate depot release of therapeutic agents. Some SBPs may release therapeutic agents according to near zero-order kinetics. In some embodiments, SBPs may release therapeutic agents according to first-order kinetics. In some embodiments, therapeutic agent release rate may be modulated by preparing SBP depots with modification of one or more of density, loading, drying method, silk fibroin molecular weight, and silk fibroin concentration.

In some embodiments, SBPs are prepared to release from about 0.01% to about 1%, from about 0.05% to about 2%, from about 1% to about 5%, from about 2% to about 10%, from about 3% to about 15%, from about 4% to about 20%, from about 5% to about 25%, from about 6% to about 30%, from about 7% to about 35%, from about 8% to about 40%, from about 9% to about 45%, from about 10% to about 50%, from about 12% to about 55%, from about 14% to about 60%, from about 16% to about 65%, from about 18% to about 70%, from about 20% to about 75%, from about 22% to about 80%, from about 24% to about 85%, from about 26% to about 90%, from about 28% to about 95%, from about 30% to about 100% of the total amount of therapeutic or macromolecular therapeutic agent to be delivered.

In some embodiments, the SBPs (e.g. hydrogels) demonstrate a sustained release of a therapeutic agent, with near steady state concentrations. In some embodiments, the sustained release is at a level at or near the effective concentration. In some embodiments, the sustained release is at greater than or equal to the effective concentration. In some embodiments the effective concentration is the IC₅₀, the EC₅₀, or the EC₈₀.

In some embodiments, use of SBPs for oral delivery of therapeutic agents (e.g., small molecules, biologics) may decrease the Cmax (maximum serum concentration) of the therapeutic agent.

### Delivery

SBPs may be delivered to cells, tissues, organs and/or organisms in naked form. As used herein in, “naked” delivery refers to delivery of an active agent with minimal or with no additional formulation or modification. Naked SBPs may be delivered to cells, tissues, organs and/or organisms using routes of administration known in the art and described herein. In some embodiments, naked delivery may include formulation in a simple buffer such as saline, phosphate buffer, or PBS.

In some embodiments, SBPs may be prepared with one or more cell penetration agents, pharmaceutically acceptable carriers, delivery agents, bioerodible or biocompatible polymers, solvents, and/or sustained-release delivery depots. SBPs may be delivered to cells using routes of administration known in the art and described herein. In some embodiments, SBPs may be formulated for direct delivery to organs or tissues in any of several ways in the art including, but not limited to, direct soaking or bathing, via a catheter, by gels, powder, ointments, creams, gels, lotions, and/or drops, or by using substrates (e.g., fabric or biodegradable materials) coated or impregnated with SBPs.

### Detectable Agents and Labels

In some embodiments, SBPs described herein may be formulated with detectable labels. As used herein, the term “detectable label” refers to any incorporated compound or entity that facilitates some form of identification. Detectable labels may include, but are not limited to various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ⁸¹mKr, ⁸²Rb, ¹¹¹In, ¹²³I, ¹³³Xe, ²⁰¹Tl, ¹²⁵I, ³⁵S, ¹⁴C, ³H, or ⁹⁹mTc (e.g., as pertechnetate (technetate(VII), TcO⁴⁻)), contrast agents (e.g., gold, gold nanoparticles, gadolinium, chelated Gd, iron oxides, superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons). Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinvlsulfonyl)phenyl]naphthalimide-3,5 disulfonate; N-(4-anilino-1-naphthyl)maleimide; anthranilamide; BODIPY; Brilliant Yellow; coumarin and derivatives (e.g., coumarin, 7-amino-4-methylcoumarin (AMC, Coumarin 120), and 7-amino-4-trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and-6)-isothiocyanate (QFITC or XRITC), and fluorescamine); 2-[2-[3-[[1,3-dihydro-1,1-dimethyl-3-(3-sulfopropyl)-2H-benz[e]indol-2-ylidene]ethylidene]-2-[4-(ethoxycarbonyl)-1-piperazinyl]-1-cyclopenten-1-yl]ethenyl]-1,1-dimethyl-3-(3-sulfopropyl)-1H-benz[e]indolium hydroxide, inner salt, compound with n,ndiethylethanamine (1:1) (IR144); 5-chloro-2-[2-[3-[(5-chloro-3-ethyl-2(3H)-benzothiazolylidene)ethylidene]-2-(diphenylamino)-1-cyclopenten-1-yl]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives (e.g., pyrene, pyrene butyrate, and succinimidyl 1-pyrene); butyrate quantum dots; Reactive Red 4 (CIBACRON™ Brilliant Red 3B-A); rhodamine and derivatives (e.g., 6-carboxy-Xrhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′tetramethyl-6-carboxyrhodamine (TAMRA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); IRD 700; IRD 800; Alexa 647; La Jolta Blue; phthalo cyanine; and naphthalo cyanine.

In some embodiments, the detectable labels may include non-detectable precursors that becomes detectable upon activation (e.g., fluorogenic tetrazine-fluorophore constructs, tetrazine-BODIPY FL, tetrazine-Oregon Green 488, or tetrazine-BODIPY TMR-X) or enzyme activatable fluorogenic agents (e.g., PROSENSE® (VisEn Medical)). In vitro assays in which enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

In some embodiments, SBPs include fluorescein isothiocyanate (FITC) as a detectable label. In some embodiments, FITC is conjugated to processed silk. In some embodiments, the processed silk conjugated to FITC is silk fibroin. Conjugation of FITC to silk fibroin may be performed using the standard isothiocyanate coupling protocol. FITC can be attached to silk fibroin via the amine group. The labeled silk fibroin may be purified from the unconjugated fluorescein by gel filtration. The final ratio of labeled silk fibroin can be determined by measuring the absorbance at 280 nm and at 495 nm.

SBPs may contain both labeled SBP and free (unlabeled) SBP. In some embodiments, the ratio of labeled SBP to free (unlabeled) SBP may be about 50:1, about 20:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 7:3, about 2.5:1, about 2:1, about 1.5:1, about 1:1, about 1:1.5, about 1:2, about 1:2.5, about 3:7, about 1:3, about 1:3.5, about 1:4, about 1:4, about 1:4.5, about 1:5, about 1:5.5, about 1:6, about 1:7, about 1:7.5, about 1:8, about 1:8.5, about 1:9, about 1:9.5, about 1:10, about 1:20, or about 1:50. In some embodiments, the ratio of labeled SBP to free (unlabeled) SBP may be from about 10:1 to about 7:1, from about 8:1 to about 5:1, from about 6:1 to about 4:1, from about 5:1 to about 3:1, from about 4:1 to about 2:1, from about 3:1 to about 1.5:1, from about 2:1 to about 1:1, from about 1:1 to about 1:2, from about 1:1.5 to about 1:3, about 1:2 to about 1:4, from about 1:3 to about 1:5, from about 1:4 to about 1:6, from about 1:5 to about 1:8, or from about 1:7 to about 1:10.

### Therapeutic Devices

In some embodiments, SBPs may be or may be included in therapeutic devices. In some embodiments, therapeutic devices may be coated with SBPs described herein. Some therapeutic devices may include therapeutic agents. In some embodiments, the use of SBPs within therapeutic devices may enable the delivery of therapeutic agents via such therapeutic devices. Some therapeutic devices may include synthetic materials. In some embodiments, therapeutic devices include, but are not limited to, any of those listed in Table 6. In the Table, example categories are indicated for each therapeutic device. These categories are not limiting and each therapeutic device may fall under multiple categories (e.g., any of the categories of therapeutic devices described herein).

TABLE 6
Therapeutic devices

| Device | Example category |
|---|---|
| artificial blood vessel | augmentation |
| artificial liver | augmentation |
| artificial organ | augmentation |
| bandage | augmentation |
| breast augmentation | augmentation |
| cartilage replacement | augmentation |
| ear drum repair | augmentation |
| filler | augmentation |
| hemostatic sponge | augmentation |
| implant | augmentation |
| silk contact lens | augmentation |
| stem cell | augmentation |
| surgical mesh | augmentation |
| surgical suture | augmentation |
| tissue replacement | augmentation |
| vascular patch | augmentation |
| wound dressing | augmentation |
| antenna | general - medical |
| applier | general - medical |
| artificial heart | general - medical |
| artificial heart valve | general - medical |
| assembly | general - medical |
| balloon | general - medical |
| band | general - medical |
| barrier | general - medical |
| biosensor | general - medical |
| biotransducer | general - medical |
| breast implant | general - medical |
| cable assembly | general - medical |
| caliper | general - medical |
| capacitor | general - medical |
| carrier | general - medical |
| clamp | general - medical |
| cochlear implant | general - medical |
| cone | general - medical |
| connector | general - medical |
| corneal implant | general - medical |
| coronary stent | general - medical |
| cryotome | general - medical |
| degradable device | general - medical |
| delivery device | general - medical |
| dental implant | general - medical |
| dermatome | general - medical |
| detector | general - medical |
| diagnostic device | general - medical |
| dilator | general - medical |
| diode | general - medical |
| discharge device | general - medical |
| display technology | general - medical |
| distractor | general - medical |
| drill bit | general - medical |
| electronic device | general - medical |
| forceps | general - medical |
| gastric stimulator | general - medical |
| graft | general - medical |
| grasper | general - medical |
| harmonic scalpel | general - medical |
| hemostatic device | general - medical |
| imaging apparatus | general - medical |
| implant | general - medical |
| implant for continuous drug delivery | general - medical |
| implantable cardioverter-defibrillator | general - medical |
| integrated circuit | general - medical |
| intraocular lens | general - medical |
| intrauterine device | general - medical |
| lancet | general - medical |
| LIGASURE ™ | general - medical |
| liner | general - medical |
| magnetic or inductive device | general - medical |
| magnetic resonance imaging apparatus | general - medical |
| mechanical assembly | general - medical |
| medical device | general - medical |
| memristor | general - medical |
| module | general - medical |
| needle | general - medical |
| nerve stimulator | general - medical |
| network | general - medical |
| neurostimulator | general - medical |
| occluder | general - medical |
| optoelectronic device | general - medical |
| pacemaker | general - medical |
| patch | general - medical |
| pen | general - medical |
| piezoelectric device | general - medical |
| pin | general - medical |
| pipe | general - medical |
| plate | general - medical |
| positioner | general - medical |
| power source | general - medical |
| probe | general - medical |
| prosthesis | general - medical |
| prosthetic | general - medical |
| protection device | general - medical |
| removable device | general - medical |
| resistor | general - medical |
| retractor | general - medical |
| rod | general - medical |
| rongeur | general - medical |
| rope | general - medical |
| ruler | general - medical |
| scalpel | general - medical |
| scope | general - medical |
| screw | general - medical |
| semiconductor | general - medical |
| sensor | general - medical |
| solution | general - medical |
| specula or speculum | general - medical |
| stent | general - medical |
| stopper | general - medical |
| sterotactic device | general - medical |
| suction tip | general - medical |
| suction tube | general - medical |
| surgical device | general - medical |
| surgical mesh | general - medical |
| surgical scissor | general - medical |
| surgical staple | general - medical |
| suture | general - medical |
| switch | general - medical |
| temperature sensor | general - medical |
| terminal | general - medical |
| tie | general - medical |
| tip | general - medical |
| transducer | general - medical |
| transistor | general - medical |
| tube | general - medical |
| tympanostomy tube | general - medical |
| ultrasound tissue disruptor | general - medical |
| vacuum tube | genera! - medical |
| vacuum valve | general - medical |
| ventilation system | general - medical |
| water balloon | general - medical |
| wire | general - medical |
| bleb | ocular device |
| gel | ocular device |
| gel that hardens after implantation | ocular device |
| implant | ocular device |
| lacrimal plug | ocular device |
| lens | ocular device |
| plug | ocular device |
| punctal plug | ocular device |
| rod | ocular device |
| slurry | ocular device |
| slurry that hardens after implantation | ocular device |
| solid | ocular device |

In some embodiments, therapeutic devices include implants. As used herein, the term “implant” refers to a device that may be embedded in or within a carrier. Implants used in therapeutic applications are typically embedded in subjects to support, repair, replace, or enhance one or more tissues or features. In some embodiments, implants include one or more excipients and/or one or more therapeutic agents. Excipients may include, but are not limited to any of those presented in Table 1, above. Therapeutic agents may include, but are not limited to, any of those presented in Table 3, above. Implants may include depots for therapeutic agent release, as described herein. In some embodiments, implants may include one or more coatings, gels, hydrogels, scaffolds, particles, or therapeutic devices (e.g., any of those listed in Table 6, above).

Some implants may be prepared by mixing a therapeutic agent with a processed silk solution. The solution may be heated to form the hydrogel. Some hydrogels may be heated to dryness and some hydrogels may be frozen and lyophilized to form an implant. Further, implants may be compressed to slow hydration as well as to slow the release of therapeutic agent. Excipients may be incorporated into processed silk solutions prior to hydrogel formation to allow for scaffold formation during the freezing/lyophilization process. Excipients could include gelling agents such as, but are not limited to, poloxamers, PEG's, mannitol, sorbitol, etc. Rods or scaffolds may be formed from hydrogels by compression or extrusion. The rods may be formed taking into consideration the dimensions and/or properties that allow for injection through small gauge needles (e.g., with a gauge of more than 20). As non-limiting examples, SBP rods may be injectable through needles with a gauge of 20, 20.5, 21, 21.5, 22, 22.5, 23, 23.5, 24, 24.5, 25, 25.5, 26, 26.5, 27, 28, 29, 30, or more than 30. In one embodiment, SBP rods are injectable with a 21-gauge needle. In one embodiment, SBP rods are injectable with a 21-gauge needle. In one embodiment, SBP rods are injectable with a 22-gauge needle. Some rods may be appropriate for subcutaneous delivery. Some rods may be formatted for other delivery formats, which may include, but are not limited to, intravitreal, intratympanic, and intraarticular delivery.

### Ocular SBPs

SBPs described herein may include ocular SBPs. As used herein, the term “ocular SBP” refers to an SBP used in any application related to the eye. Ocular SBPs may be used in therapeutic applications. Such therapeutic applications may include treating or otherwise addressing one or more ocular indications.

Ocular SBPs may be prepared in a variety of formats. Some ocular SBPs are prepared in the shape of a rod. Some ocular SBPs may be in the form of a lyophilized powder. Some ocular SBPs are in the form of a hydrogel. Other ocular SBPs may be in the form of a solution. Ocular SBPs may include ocular therapeutic agents. The ocular therapeutic agents may include any of those described herein. In some embodiments, ocular therapeutic agents include one or more of processed silk, biological agents, small molecules, proteins, NSAIDs, and VEGF-related agents. Ocular therapeutic agent proteins may include, but are not limited to, lysozyme, bovine serum albumin (BSA), bevacizumab, or VEGF-related agents. NSAIDs may include, but are not limited to, aspirin, celecoxib, diclofenac, diflunisal, etodolac, ibuprofen, indomethacin, ketoprofen, ketorolac, nabumetone, naproxen, oxaprozin, piroxicam, salsalate, sulindac, carprofen, deracoxib, fenoprofen, firocoxib, flurbirofen, mefenamic acid, meloxicam, robenacoxib, and tolmetin. In some embodiments, the SBPs stabilize ocular therapeutic agents included. Ocular SBPs may include ocular therapeutic agent concentrations [expressed as percentage of ocular therapeutic agent weight contributing to total SBP weight] of from about 0.1% to about 98% (w/w). For example, SBPs may include ocular therapeutic agents at a concentration of from about 0.01% (w/w) to about 1% (w/w), from about 0.05% (w/w) to about 2% (w/v), from about 1% (w/w) to about 5% (w/w), from about 2% (w/w) to about 10% (w/w), from about 4% (w/w) to about 16% (w/w), from about 5% (w/w) to about 20% (w/w), from about 5% (w/w) to about 85% (w/w), from about 8% (w/w) to about 24% (w/w), from about 10% (w/w) to about 30% (w/w), from about 12% (w/w) to about 32% (w/w), from about 14% (w/w) to about 34% (w/w), from about 15% (w/w) to about 95% (w/w), from about 16% (w/w) to about 36% (w/w), from about 18% (w/w) to about 38% (w/v), from about 20% (w/w) to about 40% (w/w), from about 22% (w/w) to about 42% (w/w), from about 24% (w/w) to about 44% (w/w), from about 26% (w/v) to about 46% (w/w), from about 28% (w/v) to about 48% (w/v), from about 30% (w/w) to about 50% (w/w), from about 35% (w/w) to about 55% (w/w), from about 40% (w/w) to about 60% (w/w), from about 45% (w/w) to about 65% (w/w), from about 50% (w/w) to about 70% (w/w), from about 55% (w/w) to about 75% (w/w), from about 60% (w/w) to about 80% (w/w), from about 65% (w/w) to about 85% (w/w), from about 70% (w/w) to about 90% (w/w), from about 75% (w/w) to about 95% (w/w), from about 80% (w/w) to about 96% (w/w), from about 85% (w/w) to about 97% (w/w), from about 90% (w/w) to about 98% (w/w), from about 95% (w/v) to about 99% (w/w), from about 96% (w/w) to about 99.2% (w/w), or from about 97% (w/w) to about 98% (w/w). The SBPs may include a ratio of ocular therapeutic agent (by weight, volume, or concentration) to processed silk (by weight, volume, or concentration) of from about 0.001:1 to about 1:1, from about 0.005:1 to about 5:1, from about 0.01:1 to about 1:1, from about 0.01:1 to about 4.2:1, from about 0.01:1 to about 10:1, from about 0.02:1 to about 20:1, from about 0.03:1 to about 30:1, from about 0.04:1 to about 40:1, from about 0.05:1 to about 50:1, from about 0.06:1 to about 60:1, from about 0.07:1 to about 70:1, from about 0.08:1 to about 80:1, from about 0.09:1 to about 90:1, from about 0.1:1 to about 100:1, from about 0.2:1 to about 150:1, from about 0.3:1 to about 200:1, from about 0.3:1 to about 4.2:1, from about 0.4:1 to about 250:1, from about 0.5:1 to about 300:1, from about 0.6:1 to about 350:1, from about 0.7:1 to about 400:1, from about 0.8:1 to about 450:1, from about 0.9:1 to about 500:1, from about 1:1 to about 4.2:1, from about 1:1 to about 550:1, from about 2:1 to about 600:1, from about 3:1 to about 650:1, from about 4:1 to about 700:1, from about 5:1 to about 750:1, from about 6:1 to about 800:1, from about 7:1 to about 850:1, from about 8:1 to about 900:1, from about 9:1 to about 950:1, from about 10:1 to about 960:1, from about 50:1 to about 970:1, from about 100:1 to about 980:1, from about 200:1 to about 990:1, or from about 500:1 to about 1000:1. The processed silk may be or include silk fibroin.

Ocular SBPs may include one or more excipients. The excipients may include any of those described herein. In some embodiments, the excipients include one or more of lactose, sorbitol, sucrose, mannitol, lactose USP, Starch 1500, microcrystalline cellulose, Avicel, phosphate salts, sodium chloride, potassium phosphate monobasic, potassium phosphate dibasic, sodium phosphate dibasic, sodium phosphate monobasic, polysorbate 80, phosphate buffer, phosphate buffered saline, sodium hydroxide, hydrochloric acid, dibasic calcium phosphate dehydrate, tartaric acid, citric acid, fumaric acid, succinic acid, malic acid, polyvinylpyrrolidone, copolymers of vinylpyrrolidone and vinylacetate, hydroxypropylcellulose, hydroxyethylcellulose, hydroxypropylmethylcellulose, polyvinyl alcohol, polyethylene glycol, acacia, and sodium carboxymethylcellulose. SBPs may include at least one excipient at a concentration of from about 1% to about 20% (w/w). In some embodiments, SBPs include at least one excipient at a concentration of from about 0.01% to about 1%, from about 0.05% to about 2%, from about 1% to about 5%, from about 2% to about 10%, from about 3% to about 15%, from about 4% to about 20%, from about 5% to about 25%, from about 6% to about 30%, from about 7% to about 35%, from about 8% to about 40%, from about 9% to about 45%, from about 10% to about 50%, from about 12% to about 55%, from about 14% to about 60%, from about 16% to about 65%, from about 18% to about 70%, from about 20% to about 75%, from about 22% to about 80%, from about 24% to about 85%, from about 26% to about 90%, from about 28% to about 95%, from about 30% to about 96%, from about 32% to about 97%, from about 34% to about 98%, from about 36% to about 98.5%, from about 38% to about 99%, from about 40% to about 99.5%, from about 42% to about 99.6%, from about 44% to about 99.7%, from about 46% to about 99.8%, or from about 50% to about 99.9%. SBPs may include a ratio of ocular therapeutic agent (by weight, volume, or concentration) to at least one excipient (by weight, volume, or concentration) of from about 0.001:1 to about 1:1, from about 0.005:1 to about 5:1, from about 0.01:1 to about 0.5:1, from about 0.01:1 to about 10:1, from about 0.02:1 to about 20:1, from about 0.03:1 to about 30:1, from about 0.04:1 to about 40:1, from about 0.05:1 to about 50:1, from about 0.06:1 to about 60:1, from about 0.07:1 to about 70:1, from about 0.08:1 to about 80:1, from about 0.09:1 to about 90:1, from about 0.1:1 to about 100:1, from about 0.2:1 to about 150:1, from about 0.3:1 to about 200:1, from about 0.4:1 to about 250:1, from about 0.5:1 to about 300:1, from about 0.6:1 to about 350:1, from about 0.7:1 to about 400:1, from about 0.8:1 to about 450:1, from about 0.9:1 to about 500:1, from about 1:1 to about 550:1, from about 2:1 to about 600:1, from about 3:1 to about 650:1, from about 4:1 to about 700:1, from about 5:1 to about 750:1, from about 6:1 to about 800:1, from about 7:1 to about 850:1, from about 8:1 to about 900:1, from about 9:1 to about 950:1, from about 10:1 to about 960:1, from about 50:1 to about 970:1, from about 100:1 to about 980:1, from about 200:1 to about 990:1, or from about 500:1 to about 1000:1. In some embodiments, ocular SBPs contain trace amounts of excipient. In some embodiments, the excipient is phosphate buffer or phosphate buffered saline.

Ocular SBPs may have a density of from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 36 mg/mL, from about 18 mg/mL to about 38 mg/mL, from about 20 mg/mL to about 40 mg/mL, from about 22 mg/mL to about 42 mg/mL, from about 24 mg/mL to about 44 mg/mL, from about 26 mg/mL to about 46 mg/mL, from about 28 mg/mL to about 48 mg/mL, from about 30 mg/mL to about 50 mg/mL, from about 35 mg/mL to about 55 mg/mL, from about 40 mg/mL to about 60 mg/mL, from about 45 mg/mL to about 65 mg/mL, from about 50 mg/mL to about 75 mg/mL, from about 60 mg/mL to about 240 mg/mL, from about 70 mg/mL to about 350 mg/mL, from about 80 mg/mL to about 400 mg/mL, from about 90 mg/mL to about 450 mg/mL, from about 100 mg/mL to about 500 mg/mL, from about 0.01 g/mL to about 1 g/mL, from about 0.05 g/mL to about 2 g/mL, from about 0.7 g/mL to about 1.4 g/mL, from about 1 g/mL to about 5 g/mL, from about 2 g/mL to about 10 g/mL, from about 4 g/mL to about 16 g/mL, from about 5 g/mL to about 20 g/mL, from about 8 g/mL to about 24 g/mL, from about 10 g/mL to about 30 g/mL, from about 12 g/mL to about 32 g/mL, from about 14 g/mL to about 34 g/mL, from about 16 g/mL to about 36 g/mL, from about 18 g/mL to about 38 g/mL, from about 20 g/mL to about 40 g/mL, from about 22 g/mL to about 42 g/mL, from about 24 g/mL to about 44 g/mL, from about 26 g/mL to about 46 g/mL, from about 28 g/mL to about 48 g/mL, from about 30 g/mL to about 50 g/mL, from about 35 g/mL to about 55 g/mL, from about 40 g/mL to about 60 g/mL, from about 45 g/mL to about 65 g/mL, from about 50 g/mL to about 75 g/mL, from about 60 g/mL to about 240 g/mL, from about 70 g/mL to about 350 g/mL, from about 80 g/mL to about 400 g/mL, from about 90 g/mL to about 450 g/mL, or from about 100 g/mL to about 500 g/mL.

Ocular SBPs may be in the shape of a rod. Such SBPs may include a diameter of from about 0.05 μm to about 10 μm, from about 1 μm to about 20 μm, from about 2 μm to about 30 μm, from about 5 μm to about 40 μm, from about 10 μm to about 50 μm, from about 20 μm to about 60 μm, from about 30 μm to about 70 μm, from about 40 μm to about 80 μm, from about 50 μm to about 90 μm, from about 45 μm to about 100 μm, from about 50 μm to about 110 μm, from about 55 μm to about 120 μm, from about 60 μm to about 130 μm, from about 65 μm to about 140 μm, from about 70 μm to about 150 μm, from about 75 μm to about 160 μm, from about 80 μm to about 170 μm, from about 85 μm to about 180 μm, from about 90 μm to about 190 μm, from about 95 μm to about 200 μm, from about 100 μm to about 210 μm, from about 115 μm to about 220 μm, from about 125 μm to about 240 μm, from about 135 μm to about 260 μm, from about 145 μm to about 280 μm, from about 155 μm to about 300 μm, from about 165 μm to about 320 μm, from about 175 μm to about 340 μm, from about 185 μm to about 360 μm, from about 195 μm to about 380 μm, from about 205 μm to about 400 μm, from about 215 μm to about 420 μm, from about 225 μm to about 440 μm, from about 235 μm to about 460 μm, from about 245 μm to about 500 μm, from about 0.05 mm to about 2 mm, from about 0.1 mm to about 1.5 mm, from about 0.1 mm to about 3 mm, from about 0.2 mm to about 4 mm, from about 0.3 mm to about 1.2 mm, from about 0.5 mm to about 5 mm, from about 1 mm to about 6 mm, from about 2 mm to about 7 mm, or from about 5 mm to about 10 mm. SBP rods may have a length of from about 0.05 mm to about 2 mm, from about 0.1 mm to about 3 mm, from about 0.2 mm to about 4 mm, from about 0.3 mm to about 1.2 mm, from about 0.5 mm to about 5 mm, from about 1 mm to about 6 mm, from about 2 mm to about 7 mm, from about 5 mm to about 10 mm, from about 8 mm to about 12 mm, from about 10 mm to about 15 mm, from about 12 mm to about 18 mm, from about 15 mm to about 25 mm, or from about 20 mm to about 30 mm.

Ocular SBPs may be hydrogels. Such SBPs may include at least one excipient selected from one or more of sorbitol, triethylamine, 2-pyrrolidone, alpha-cyclodextrin, benzyl alcohol, beta-cyclodextrin, dimethyl sulfoxide, dimethylacetamide (DMA), dimethylformamide, ethanol, gamma-cyclodextrin, glycerol, glycerol formal, hydroxypropyl beta-cyclodextrin, kolliphor 124, kolliphor 181, kolliphor 188, kolliphor 407, kolliphor EL (cremaphor EL), cremaphor RH 40, cremophor RH 60, dalpha-tocopherol, PEG 1000 succinate, polysorbate 20, polysorbate 80, solutol HS 15, sorbitan monooleate, poloxamer-407, poloxamer-188, Labrafil M-1944CS, Labrafil M-2125CS, Labrasol, Gellucire 44/14, Softigen 767, mono- and di-fatty acid esters of PEG 300, PEG 400, or PEG 1750, kolliphor RH60, N-methyl-2-pyrrolidone, castor oil, corn oil, cottonseed oil, olive oil, peanut oil, peppermint oil, safflower oil, sesame oil, soybean oil, hydrogenated vegetable oils, hydrogenated soybean oil, medium chain triglycerides of coconut oil, medium chain triglycerides of palm seed oil, beeswax, d-alpha-tocopherol, oleic acid, medium-chain mono-glycerides, medium-chain di-glycerides, alpha-cyclodextrin, betacyclodextrin, hydroxypropyl-beta-cyclodextrin, sulfo-butylether-beta-cyclodextrin, hydrogenated soy phosphatidylcholine, distearoylphosphatidylglycerol, L-alphadimyristoylphosphatidylcholine, L-alpha-dimyristoylphosphatidylglycerol, PEG 300, PEG 300 caprylic/capric glycerides (Softigen 767), PEG 300 linoleic glycerides (Labrafil M-2125CS), PEG 300 oleic glycerides (Labrafil M-1944CS), PEG 400, PEG 400 caprylic/capric glycerides (Labrasol), polyoxyl 40 stearate (PEG 1750 monosterate), polyoxyl 8 stearate (PEG 400 monosterate), polysorbate 20, polysorbate 80, polyvinyl pyrrolidone, propylene carbonate, propylene glycol, solutol HS15, sorbitan monooleate (Span 20), sulfobutylether-beta-cyclodextrin, transcutol, triacetin, 1-dodecylazacyclo-heptan-2-one, caprolactam, castor oil, cottonseed oil, ethyl acetate, medium chain triglycerides, methyl acetate, oleic acid, safflower oil, sesame oil, soybean oil, tetrahydrofuran, glycerin, and PEG 4 kDa. The SBPs may have an osmotic concentration of from about 1 mOsm to about 10 mOsm, from about 2 mOsm to about 20 mOsm, from about 3 mOsm to about 30 mOsm, from about 4 mOsm to about 40 mOsm, from about 5 mOsm to about 50 mOsm, from about 6 mOsm to about 60 mOsm, from about 7 mOsm to about 70 mOsm, from about 8 mOsm to about 80 mOsm, from about 9 mOsm to about 90 mOsm, from about 10 mOsm to about 100 mOsm, from about 15 mOsm to about 150 mOsm, from about 25 mOsm to about 200 mOsm, from about 35 mOsm to about 250 mOsm, from about 45 mOsm to about 300 mOsm, from about 55 mOsm to about 350 mOsm, from about 65 mOsm to about 400 mOsm, from about 75 mOsm to about 450 mOsm, from about 85 mOsm to about 500 mOsm, from about 125 mOsm to about 600 mOsm, from about 175 mOsm to about 700 mOsm, from about 225 mOsm to about 800 mOsm, from about 275 mOsm to about 285 mOsm, from about 280 mOsm to about 900 mOsm, or from about 325 mOsm to about 1000 mOsm. In some embodiments, ocular SBP hydrogels may be runnier or thinner than hydrogels used to other indications (e.g., tissue repair).

Ocular SBPs may have a pH from about 3 to about 10. In some embodiments, the pH is from about 3 to about 6, from about 6 to about 8, or from about 8 to about 10. In some embodiments, the pH of the SBP is about 7.4.

Ocular SBPs may include silk fibroin. The silk fibroin may be included at a concentration (w/w or w/v) of 0.01% to about 1%, from about 0.05% to about 2%, from about 0.1% to about 30%, from about 1% to about 5%, from about 2% to about 10%, from about 3% to about 15%, from about 4% to about 20%, from about 5% to about 25%, from about 6% to about 30%, from about 7% to about 35%, from about 8% to about 40%, from about 9% to about 45%, from about 10% to about 50%, from about 12% to about 55%, from about 14% to about 60%, from about 16% to about 65%, from about 18% to about 70%, from about 20% to about 75%, from about 22% to about 80%, from about 24% to about 85%, from about 26% to about 90%, from about 28% to about 95%, from about 30% to about 96%, from about 32% to about 97%, from about 34% to about 98%, from about 36% to about 98.5%, from about 38% to about 99%, from about 40% to about 99.5%, from about 42% to about 99.6%, from about 44% to about 99.7%, from about 46% to about 99.8%, or from about 50% to about 99.9%. SBPs may include a ratio of silk fibroin (by weight, volume, or concentration) to at least one excipient and/or ocular therapeutic agent (by weight, volume, or concentration) of from about 0.001:1 to about 1:1, from about 0.005:1 to about 5:1, from about 0.01:1 to about 0.5:1, from about 0.01:1 to about 10:1, from about 0.02:1 to about 20:1, from about 0.03:1 to about 30:1, from about 0.04:1 to about 40:1, from about 0.05:1 to about 50:1, from about 0.06:1 to about 60:1, from about 0.07:1 to about 70:1, from about 0.08:1 to about 80:1, from about 0.09:1 to about 90:1, from about 0.1:1 to about 100:1, from about 0.2:1 to about 150:1, from about 0.3:1 to about 200:1, from about 0.4:1 to about 250:1, from about 0.5:1 to about 300:1, from about 0.6:1 to about 350:1, from about 0.7:1 to about 400:1, from about 0.8:1 to about 450:1, from about 0.9:1 to about 500:1, from about 1:1 to about 550:1, from about 2:1 to about 600:1, from about 3:1 to about 650:1, from about 4:1 to about 700:1, from about 5:1 to about 750:1, from about 6:1 to about 800:1, from about 7:1 to about 850:1, from about 8:1 to about 900:1, from about 9:1 to about 950:1, from about 10:1 to about 960:1, from about 50:1 to about 970:1, from about 100:1 to about 980:1, from about 200:1 to about 990:1, or from about 500:1 to about 1000:1. In some embodiments, ocular SBPs contain trace amounts of excipient. In some embodiments, the excipient is phosphate buffer or phosphate buffered saline.

SBP viscosity may be modulated by modulating silk fibroin molecular weight and/or concentration. In some embodiments, SBP viscosity increases with increasing levels of silk fibroin. In some embodiments, SBP viscosity may be tuned by the molecular weight of processed silk, as defined by the minute boil. In some embodiments, the viscosity of an SBP is proportional to the molecular weight of the processed silk. In some embodiments, the viscosity of an SBP is from about 7 Pa s⁻ to about 170 Pa s⁻¹. In some embodiments, the viscosity of an SBP is from about 5 Pa s⁻ to about 200 Pa s⁻¹. In some embodiments, the viscosity of an SBP is from about 5 Pa s⁻ to about 25 Pa s⁻¹, from about 25 Pa s⁻ to about 50 Pa s⁻¹, from about 50 Pa s⁻ to about 75 Pa s⁻¹, from about 75 Pa s⁻ to about 100 Pa s⁻¹, from about 100 Pa s⁻ to about 125 Pa s⁻¹, from about 125 Pa s⁻ to about 150 Pa s⁻¹, from about 150 Pa s to about 175 Pa s⁻¹, or from about 175 Pa s⁻ to about 200 Pa s⁻¹. In some embodiments, the stiffness of the SBP may be tuned with the molecular weight of the processed silk. In some embodiments, a preparation of an SBP from processed silk with a longer boiling time may enhance the stiffness of the SBP. In some embodiments, the viscosity and/or the stiffness of the SBP may be modulated without altering the release kinetics of a therapeutic agent from the SBP.

In some embodiments, ocular SBPs are formulated for intraocular administration. In some embodiments, ocular SBPs are formulated for one or more of intravitreal administration, intraretinal administration, intracorneal administration, intrascleral administration, punctal administration, administration to the anterior sub-Tenon's, suprachoroidal administration, administration to the posterior sub-Tenon's, subretinal administration, administration to the fornix, administration to the lens, administration to the anterior segment, administration to the posterior segment, macular administration, and intra-aqueous humor administration. Ocular SBPs may be biocompatible, well tolerated, and/or non-immunogenic.

In some embodiments, the present disclosure provides methods of treating subjects by contacting them with ocular SBPs. The subjects may have, may be suspected of having, and/or may be at risk for developing one or more ocular indications. Such ocular indications may include any of those described herein. In some embodiments, ocular indications include inflammation. In some embodiments, ocular indications include one or more of an infection, refractive errors, macular edema, age related macular degeneration, cystoid macular edema, cataracts, diabetic retinopathy (proliferative and non-proliferative), glaucoma, amblyopia, strabismus, color blindness, cytomegalovirus retinitis, keratoconus, diabetic macular edema (proliferative and non-proliferative), low vision, ocular hypertension, retinal detachment, eyelid twitching, inflammation, uveitis, bulging eyes, dry eye disease, floaters, xerophthalmia, diplopia, Graves' disease, night blindness, eye strain, red eyes, nystagmus, presbyopia, excess tearing, retinal disorder, conjunctivitis, cancer, corneal ulcer, corneal abrasion, snow blindness, scleritis, keratitis, Thygeson's superficial punctate keratopathy, corneal neovascularization, Fuch's dystrophy, keratoconjunctivitis sicca, iritis, chorioretinal inflammation (e.g. chorioretinitis, choroiditis, retinitis, retinochoroiditis, pars planitis, Harada's disease, aniridia, macular scars, solar retinopathy, choroidal degeneration, choroidal dystrophy, choroideremia, gyrate atrophy, choroidal hemorrhage, choroidal detachment, retinoschisis, hypertensive retinopathy, Bull's eye maculopathy, epiretinal membrane, peripheral retinal degeneration, hereditary retinal dystrophy, retinitis pigmentosa, retinal hemorrhage, retinal vein occlusion, and separation of retinal layers.

In some embodiments, the ocular indication is DME. In some embodiments, the ocular indication is diabetic retinopathy. In some embodiments, the ocular indication is non-proliferative diabetic retinopathy.

In some embodiments, the SBPs of the present disclosure may be administered to treat subjects with diabetic macular edema. In some embodiments, the SBPs of the present disclosure may be used to treat diabetic retinopathy in subjects with DME. In some embodiments, DME is non-proliferative. In some embodiments, diabetic retinopathy is non-proliferative (NPDR). In some embodiments SBPs of the present disclosure may be used to achieve the sustained release of one or more known NSAID with intravitreal triamcinolone (IVT). In some embodiments, SBPs of the present disclosure may be used to achieve the sustained release of one or more known NSAID with intravitreal triamcinolone acetonide. In some embodiments, the SBP comprises one or more NSAID and is administered alongside intravitreal triamcinolone or triamcinolone acetonide. In some embodiments, the SBP comprises one or more NSAID and triamcinolone or triamcinolone acetonide. In some embodiments, the mechanism of action of the treatment is novel compared to that of existing treatments of NPDR (e.g. VEGF or steroids). In some embodiments, the mechanism of action of the treatment is additive to that of VEGF antagonist with respect to the mean improvement in BCVA ETDRS. In some embodiments, the mechanism of action of the treatment is additive to that of VEGF alone with respect to the mean improvement in BCVA ETDRS. In some embodiments, the efficacy of the treatment is similar to that of intravitreal triamcinolone or triamcinolone acetonide. In some embodiments, the efficacy of the treatment is improved over that of intravitreal triamcinolone or triamcinolone acetonide. In some embodiments, the safety of the treatment is improved over that of intravitreal triamcinolone or triamcinolone acetonide. In some embodiments, the adverse event burden is better or similar to that of a VEGF antagonist. In some embodiments, the adverse event burden is better than that of an IVT steroid. In some embodiments, the SBP is administered via injection. In some embodiments, the SBP is administered every 6 months. In some embodiments, the SBP is administered every 3 months.

In some embodiments, subjects with NPDR may be evaluated as a part of a population of subjects with DME. In some embodiments, SBPs of the present disclosure may be administered adjunctive with a VEGF antagonist. In some embodiments, SPBs of the present disclosure may be administered adjunctive with VEGF and/or VEGF sub-optimal responders. In some embodiments, treatment of DME and DME in subjects with NPDR may be measured by refraction and Best Corrected Visual Acuity using Early Treatment in Diabetic Retinopathy Study Methodology (BCVA ETDRS). In some embodiments, treatment is measured by the mean change in BCVA ETDRS score at 9 months. In some embodiments, the treatment with SBPs results in an improvement in NPDR score. In some embodiments, the improvement is at least two steps.

In further embodiments, ocular SBPs may be prepared as eye drops for the treatment of dry eye disease, as described in U.S. Pat. No. 9,394,355, the contents of which are hereby incorporated by reference in their entirety, or formulated for the treatment of corneal injury, as described in International Publication Numbers W2017200659 and WO2018031973; Abdel-Naby et al. (2017) Invest Ophthalmol Vis Sci; 58(3):1425-1433; and Abdel-Naby et al. (2017) PLoS One; 12(11):e0188154, the contents of each of which are hereby incorporated by reference in their entirety.

Methods of treating subjects with ocular SBPs may include one or more of oral administration, intravenous administration, topical administration, and ocular administration. Ocular administration may include one or more of intravitreal administration, intraretinal administration, intracorneal administration, intrascleral administration, administration to the anterior segment, administration to the posterior segment, and intra-aqueous humor administration. In some embodiments, the SBP adheres to the ocular surface. In some embodiments, the SBP adheres to the ocular surface in a manner similar to a mucin layer. Intravitreal administration may be performed at any injection site that would enable the administration of the SBP to the intravitreal space. Intravitreal administration may include intravitreal injection. Intravitreal injection may be performed by pushing a wire through a syringe and needle or cannula loaded with ocular SBP. The wire may be pushed until it extends past the needle or cannula.

In some embodiments, the residence time of an SBP will be analyzed after SBP administration, using any method known to one skilled in the art. In some embodiments, the efficacy of an SBP will be analyzed after SBP administration, using any method known to one skilled in the art. In some embodiments, the pharmacokinetics of an SBP will be analyzed after SBP administration, using any method known to one skilled in the art. In some embodiments, the irritability of an SBP will be analyzed after SBP administration, using any method known to one skilled in the art. In some embodiments, the use of an SBP to treat irritation will be analyzed after SBP administration, using any method known to one skilled in the art. In some embodiments, the toxicity of an SBP will be analyzed after SBP administration, using any method known to one skilled in the art.

Ocular SBPs may be used to treat subjects by delivering ocular therapeutic agents at a dose of from about 0.01 μg to about 1 μg, from about 0.05 μg to about 2 μg, from about 1 μg to about 5 μg, from about 2 μg to about 10 μg, from about 4 μg to about 16 μg, from about 5 μg to about 20 μg, from about 8 μg to about 24 μg, from about 10 μg to about 30 μg, from about 12 μg to about 32 μg, from about 14 μg to about 34 μg, from about 16 μg to about 36 μg, from about 18 μg to about 38 μg, from about 20 μg to about 40 μg, from about 22 μg to about 42 μg, from about 24 μg to about 44 μg, from about 26 μg to about 46 μg, from about 28 μg to about 48 μg, from about 30 μg to about 50 μg, from about 35 μg to about 55 μg, from about 40 μg to about 60 μg, from about 45 μg to about 65 μg, from about 50 μg to about 75 μg, from about 60 μg to about 240 μg, from about 70 μg to about 350 μg, from about 80 μg to about 400 μg, from about 90 μg to about 450 μg, from about 100 μg to about 500 μg, from about 200 μg to about 750 μg, from about 300 μg to about 1000 μg, from about 1 μg to about 5000 μg, or from about 500 μg to about 5000 μg. In some embodiments, subjects are contacted with a dose of ocular therapeutic agents sufficient to achieve concentrations in subject eyes (or components of subject eyes) greater than or equal to the effective concentration for such ocular therapeutic agents. The concentrations may be 1.5-fold, 2-fold, 4-fold, 5-fold, 10-fold, or more than 10-fold greater than the effective concentration.

In some embodiments, contacting subjects with ocular SBPs results in ocular therapeutic agent concentrations in subject eyes of from about 0.01 ng/mL to about 70,000 ng/ml. In some embodiments, the resulting concentration in subject eyes is from about 0.01 ng/mL to about 1 ng/mL, from about 0.05 ng/mL to about 2 ng/mL, from about 1 ng/mL to about 5 ng/mL, from about 2 ng/mL to about 10 ng/mL, from about 4 ng/mL to about 16 ng/mL, from about 5 ng/mL to about 20 ng/mL, from about 8 ng/mL to about 24 ng/mL, from about 10 ng/mL to about 30 ng/mL, from about 12 ng/mL to about 32 ng/mL, from about 14 ng/mL to about 34 ng/mL, from about 16 ng/mL to about 36 ng/mL, from about 18 ng/mL to about 38 ng/mL, from about 20 ng/mL to about 40 ng/mL, from about 22 ng/mL to about 42 ng/mL, from about 24 ng/mL to about 44 ng/mL, from about 26 ng/mL to about 46 ng/mL, from about 28 ng/mL to about 48 ng/mL, from about 30 ng/mL to about 50 ng/mL, from about 35 ng/mL to about 55 ng/mL, from about 40 ng/mL to about 60 ng/mL, from about 45 ng/mL to about 65 ng/mL, from about 50 ng/mL to about 75 ng/mL, from about 60 ng/mL to about 240 ng/mL, from about 70 ng/mL to about 350 ng/mL, from about 80 ng/mL to about 400 ng/mL, from about 90 ng/mL to about 450 ng/mL, from about 100 ng/mL to about 500 ng/mL, from about 0.01 μg/mL to about 1 μg/mL, from about 0.05 μg/mL to about 2 μg/mL, from about 1 μg/mL to about 5 μg/mL, from about 2 μg/mL to about 10 μg/mL, from about 4 μg/mL to about 16 μg/mL, from about 5 μg/mL to about 20 μg/mL, from about 8 μg/mL to about 24 μg/mL, from about 10 μg/mL to about 30 μg/mL, from about 12 μg/mL to about 32 μg/mL, from about 14 μg/mL to about 34 μg/mL, from about 16 μg/mL to about 36 μg/mL, from about 18 μg/mL to about 38 μg/mL, from about 20 μg/mL to about 40 μg/mL, from about 22 μg/mL to about 42 μg/mL, from about 24 μg/mL to about 44 μg/mL, from about 26 μg/mL to about 46 μg/mL, from about 28 μg/mL to about 48 μg/mL, from about 30 μg/mL to about 50 μg/mL, from about 35 μg/mL to about 55 μg/mL, from about 40 μg/mL to about 60 μg/mL, from about 45 μg/mL to about 65 μg/mL, from about 50 μg/mL to about 75 μg/mL, from about 60 μg/mL to about 240 μg/mL, from about 70 μg/mL to about 350 μg/mL, from about 80 μg/mL to about 400 μg/mL, from about 90 μg/mL to about 450 μg/mL, from about 100 μg/mL to about 500 μg/mL, from about 0.01 mg/mL to about 1 mg/mL, from about 0.05 mg/mL to about 2 mg/mL, from about 1 mg/mL to about 5 mg/mL, from about 2 mg/mL to about 10 mg/mL, from about 4 mg/mL to about 16 mg/mL, from about 5 mg/mL to about 20 mg/mL, from about 8 mg/mL to about 24 mg/mL, from about 10 mg/mL to about 30 mg/mL, from about 12 mg/mL to about 32 mg/mL, from about 14 mg/mL to about 34 mg/mL, from about 16 mg/mL to about 35 mg/mL, or from about 35 mg/mL to about 70 mg/mL. The ocular therapeutic agent concentration in subject eyes may include concentration in one or more eye components. The components may include, but are not limited to, the aqueous humor, vitreous humor, retina, choroid, sclera, lens, fornix, conjunctiva, lacrimal punctum, capsule of Tenon, iris, pupal, cornea, ciliary muscle, fovea, optic nerve, macula, blood vessel, anterior chamber, posterior chamber, and sub-tenon space. In some embodiments, contacting subjects with ocular SBPs may result in ocular therapeutic agent concentration in subject aqueous humor of from about 0.01 ng/mL to about 2.0 ng/mL. In some embodiments, vitreous humor concentration may be from about 10 ng/mL to about 20,000 ng/ml. In some embodiments, retina and/or choroid concentrations may be from about 10 ng/mL to about 70,000 ng/mL. Ocular therapeutic agent levels may be detectable in subject eyes for at least 1 day, for at least 2 days, for at least 3 days, for at least 1 week, for at least 2 weeks, for at least 1 month, for at least 3 months, for at least 6 months, or for at least year. In some embodiments, ocular therapeutic agent levels remain at a steady level for at least 1 day, for at least 2 days, for at least 3 days, for at least 1 week, for at least 2 weeks, for at least 1 month, for at least 3 months, for at least 6 months, or for at least 1 year. In some embodiments, the concentration of the ocular therapeutic agent in the subject eye or component of the eye is at a level at or near the effective concentration. In some embodiments, the concentration of the ocular therapeutic agent in the subject eye or component of the eye is sustained at a level at or near the effective concentration. In some embodiments, the concentration of the ocular therapeutic agent in the subject eye or component of the eye is sustained at a level greater than the effective concentration. In some embodiments the effective concentration is the IC₅₀, the EC₅₀, or the EC₈₀.

In some embodiments, the ocular SBPs may be hydrogels. In some embodiments, the ocular SBPs are rods. In some embodiments, the ocular SBPs are administered via intravitreal administration. In some embodiments, the ocular SBPs are formulated with celecoxib. In some embodiments, the intravitreal administration of the ocular SBPs enables at least 6 months of sustained release at or above the effective concentration. In some embodiments the effective concentration is the IC₅₀. In some embodiments, the effective concentration is the EC₈₀. In some embodiments, the IC₅₀ is 40 nM. In some embodiments, the EC₅₀ is 1-3 μM.

In some embodiments, ocular SBPs may be used to reduce ocular pressure. In some embodiments, the intravitreal administration of the ocular SBPs results in a sustained intraocular pressure. In some embodiments, the reduced or sustained intraocular pressure may be observed for at least 1 day, at least 3 days, at least 1 week, at least 2 weeks, at least 1 month, at least 3 months, at least 4 months, at least 6 months, or at least 1 year after SBP administration.

In some embodiments, the ocular SBPs of the present disclosure are biocompatible in the ocular space. In some embodiments, administration of the ocular SBP does not cause local inflammation in the ocular space. In some embodiments, ocular SBP is tolerable in the ocular space. In some embodiments, the retinal tissue remains normal after the administration of the ocular SBP. In some embodiments, the SBPs are biocompatible and tolerable in the ocular space for at least 1 day, at least 3 days, at least 1 week, at least 2 weeks, at least 1 month, at least 3 months, at least 4 months, at least 6 months, or at least 1 year.

In some embodiments, the present disclosure provides methods of delivering ocular therapeutic agents to subjects by contacting subject eyes with ocular SBPs. Such ocular SBPs may be prepared by combining processed silk with ocular therapeutic agents. The SBPs may be prepared with a low temperature, aqueous processing procedure. The SBPs may be prepared as rods. The rods may be prepared by extrusion through a tube. The tube may be a needle. Extrusion may be carried out using a syringe. Ocular therapeutic agents may be delivered to subject eyes by release from SBPs while SBPs are in contact with the eyes. Release of ocular therapeutic agents from SBPs may be modulated by one or more of silk fibroin concentration, silk fibroin molecular weight, SBP volume, method used to dry SBPs, ocular therapeutic agent molecular weight, and inclusion of at least one excipient. Methods used to dry SBPs may include one or more of oven drying, lyophilizing, and air drying. In some embodiments, an ocular SBP is prepared as a gel, before drying to obtain the SBP in a rod format. Ocular SBP rods may include ocular therapeutic agents and silk fibroin at a w/w ratio of from about 1 to about 5.

Release of ocular therapeutic agents from ocular SBPs may occur at a rate that includes an initial burst. From about 0.01% to about 100% of ocular therapeutic agents may be released from SBPs during an initial release period associated with the initial burst. In some embodiments, from about 5% to about 20% of ocular therapeutic agents may be released from SBPs during an initial release period associated with the initial burst. Release of ocular therapeutic agent from SBPs may include a daily release percentage of from about 0.1% (w/w) to about 5% (w/w). In some embodiments the release rates of the therapeutic agents are tunable. In some embodiments, the release rates are tunable on the order of days to weeks. In some embodiments the release rates are tunable on the order of weeks to months.

In some embodiments, the release rates are tuned by varying the API loading, the silk fibroin molecular weight, the silk fibroin concentration, drying method of the SBP, and the density of the ocular SBP during formulation. In some embodiments, the release kinetics of an API from an SBP may be tuned by the density of the SBP. In some embodiments, the daily release percentage and the initial burst may be decreased by preparation of a denser SBP. In some embodiments, the release kinetics of an API from an SBP may be tuned by the concentration of processed silk in the SBP. In some embodiments, the daily release percentage and the initial burst may be decreased by preparation with a higher concentration of processed silk. In some embodiments, the release of an API from an ocular SBP is biphasic, in that the release rate changes between two portions of the study.

In some embodiments, from about 1% to about 100% of ocular therapeutic agents are released from ocular SBPs during a release period. The release period may be from about 1 day to about 10 months. The release period may begin upon contacting an eye of a subject with an SBP. The release period may be from about 1 day to about 5 months. The release period may be from about 1 day to about 6 months. In some embodiments, the API is released over a period of at least 1 day, for at least 2 days, for at least 3 days, for at least 1 week, for at least 2 weeks, for at least 1 month, for at least 3 months, for at least 6 months, or for at least 1 year. In some embodiments, 0.1%-100% of ocular therapeutic agents may be released from SBPs over release periods. In some embodiments, from about 40% to about 60% of ocular therapeutic agents may be released from SBPs over release periods. In some embodiments, the release of the therapeutic agents from ocular SBPs follows first order kinetics. In some embodiments, the release of therapeutic agents from ocular SBPs follows zero order kinetics. In some embodiments the release periods of the therapeutic agents are tunable. In some embodiments, the release rates are tunable on the order of days to weeks. In some embodiments the release periods are tunable on the order of weeks to months. In some embodiments, the release periods are tuned by varying the API loading, the silk fibroin molecular weight, the silk fibroin concentration, and the density of the ocular SBP during formulation. In some embodiments, the therapeutic agent is an NSAID. In some embodiments, the SBP formulated with NSAID has a release period of at least 1 day, at least 3 days, at least 1 week, at least 1 month, at least 3 months, at least 6 months, or at least 1 year in vitro. In some embodiments, the SBP formulated with NSAID has a release period of at least 1 day, at least 3 days, at least 1 week, at least 1 month, at least 3 months, at least 6 months, or at least 1 year in vivo.

In some embodiments, the ocular SBP is a rod, and the release duration of CXB is related to the rod density. In some embodiments, increased density of a rod results in increased release times. In some embodiments, the density of the rod is tuned by varying the starting concentration of the silk-fibroin used during formulation. In some embodiments, the rods with a density below 1.0 g/mL reach complete release about 64 days or less. In some embodiments, the rods with a density between 1.0 g/mL and 1.1 g/mL reach complete release in about 98 days. In some embodiments, the rods with a density above 1.1 g/mL reach complete release in greater than 98 days.

### III. Agricultural Applications and Products

In some embodiments, SBPs are prepared for use in agriculture. As used herein, the term “agriculture” refers to the cultivation of plants and animals to produce products useful for individual, communal, industrial, or commercial purposes. SBPs may be agricultural compositions. In some embodiments, SBPs may include an agricultural composition. As used herein, the term “agricultural composition” refers to any substance used in or produced by agriculture. In some embodiments, SBPs may be used to improve the growth, production, the shelf-life and stability of agricultural products. As used herein, the term “agriculture product” refers to any product of agriculture (e.g., food, medicines, materials, biofuels, etc.). In some embodiments. SBPs may be used in a variety of agricultural applications. As used herein, the term “agricultural application” refers to any method used to improve, promote or increase the production of products obtained through the cultivation of plants and animals, for the benefit of individuals, communities, or commercial entities.

In some embodiments, agricultural compositions described herein are used for agricultural and environmental development. In some embodiments, SBPs may be used to improve the growth and production of agricultural products. These agricultural products may be plants, animals, plant agricultural products, or animal agricultural products. In some embodiments, SBP administration may result in increased weight, biomass, growth, offspring production, product levels, and/or product size of one or more agricultural products.

### Cargo

In some embodiments, SBP agricultural compositions are used to facilitate delivery of cargo that enhance agricultural product health, yield, half-life and/or stability. In some embodiments, SBPs may be the cargos. In some embodiments, cargos may include, but are not limited to, therapeutic agents, small molecules, chemicals, nutrients, micronutrients, macronutrients, pest control agents, pesticides, antibiotics, antifungal, fungicide, virus, virus fragment, virus particle, herbicide, insecticide, fertilizers, pH modulators, soil stabilizers, and flowability agents. In some embodiments, the cargo is stabilized by formulation within an SBP agricultural composition. In some embodiments, the efficacy of the cargo is improved by formulation within an SBP agricultural composition.

In some embodiments, cargos for use in SBPs may be selected from any of those listed in Table 7.

TABLE 7
Cargo

| Payload | Category |
|---|---|
| amikacin | antibiotic |
| amoxicillin | antibiotic |
| ampicillin | antibiotic |
| azithromycin | antibiotic |
| azlocillin | antibiotic |
| aztreonam | antibiotic |
| capreomycin | antibiotic |
| carbenicillin | antibiotic |
| cefaclor | antibiotic |
| cefadroxil | antibiotic |
| cefalexin | antibiotic |
| cefalothin | antibiotic |
| cefamandole | antibiotic |
| cefazolin | antibiotic |
| cefdinir | antibiotic |
| cefditoren | antibiotic |
| cefepime | antibiotic |
| cefixime | antibiotic |
| cefoperazone | antibiotic |
| cefotaxime | antibiotic |
| cefoxitin | antibiotic |
| cefpodoxime | antibiotic |
| cefprozil | antibiotic |
| ceftaroline fosamil | antibiotic |
| ceftazidime | antibiotic |
| ceftibuten | antibiotic |
| ceftizoxime | antibiotic |
| ceftobiprole | antibiotic |
| ceftriaxone | antibiotic |
| cefuroxime | antibiotic |
| cilastatin | antibiotic |
| ciprofolaxin | antibiotic |
| clarithromycin | antibiotic |
| clindamycin | antibiotic |
| clofazimine | antibiotic |
| cloxacillin | antibiotic |
| cycloserine | antibiotic |
| dalbavancin | antibiotic |
| dapsone | antibiotic |
| daptomycin | antibiotic |
| demeclocycline | antibiotic |
| dicloxacillin | antibiotic |
| dirithromycin | antibiotic |
| doripenem | antibiotic |
| doxycycline | antibiotic |
| enoxacin | antibiotic |
| ertapenem | antibiotic |
| ethambutol | antibiotic |
| ethionamide | antibiotic |
| flucloxacillin | antibiotic |
| furazolidone | antibiotic |
| gatifloxacin | antibiotic |
| geldanamycin | antibiotic |
| gemifloxacin | antibiotic |
| gentamicin | antibiotic |
| grepafloxacin | antibiotic |
| herbimycin | antibiotic |
| imipeneum | antibiotic |
| isoniazid | antibiotic |
| kanamycin | antibiotic |
| levofloxacin | antibiotic |
| linezolid | antibiotic |
| linomycin | antibiotic |
| lomefloxacin | antibiotic |
| loracarbef | antibiotic |
| mafenide | antibiotic |
| meropenem | antibiotic |
| methicillin | antibiotic |
| mezlocillin | antibiotic |
| minocycline | antibiotic |
| moxifloxacin | antibiotic |
| nafcillin | antibiotic |
| nalidixic acid | antibiotic |
| neomycin | antibiotic |
| netilmicin | antibiotic |
| nitrofurantoin | antibiotic |
| norfloxacin | antibiotic |
| ofloxacin | antibiotic |
| oritavancin | antibiotic |
| oxacillin | antibiotic |
| oxytetracycline | antibiotic |
| paromomycin | antibiotic |
| penicillin G | antibiotic |
| penicillin V | antibiotic |
| piperacillin | antibiotic |
| posizolid | antibiotic |
| pyrazinamide | antibiotic |
| radezolid | antibiotic |
| rifampicin | antibiotic |
| rifaximin | antibiotic |
| roxithromycin | antibiotic |
| sparfloxacin | antibiotic |
| spectinomycin | antibiotic |
| spiramycin | antibiotic |
| sulfacetamide | antibiotic |
| sulfadiazine | antibiotic |
| sulfadimethoxine | antibiotic |
| sulfamethizole | antibiotic |
| sulfamethoxazole | antibiotic |
| sulfanilimide | antibiotic |
| sulfasalazine | antibiotic |
| sulfisoxazole | antibiotic |
| teicoplanin | antibiotic |
| telavancin | antibiotic |
| telithromycin | antibiotic |
| temafloxacin | antibiotic |
| temocillin | antibiotic |
| ticarcillin | antibiotic |
| tobramycin | antibiotic |
| torezolid | antibiotic |
| troleandomycin | antibiotic |
| trovafloxacin | antibiotic |
| vancomycin | antibiotic |
| erythromycin | antibiotic; endoparasiticide |
| penicillin | antibiotic; endoparasiticide |
| streptomycin | antibiotic; endoparasiticide |
| tetracycline | antibiotic; endoparasiticide |
| 5-fluorocytosine | antifungal |
| abafungin | antifungal |
| albaconazole | antifungal |
| allylamine | antifungal |
| amorolfin | antifungal |
| amphotericin B | antifungal |
| anidulafungin | antifungal |
| aurone | antifungal |
| balsam | antifungal |
| benzoic acid | antifungal |
| bifonazole | antifungal |
| butenafine | antifungal |
| butoconazole | antifungal |
| candicidin | antifungal |
| caspofungin | antifungal |
| ciclopirox | antifungal |
| clotrimazole | antifungal |
| crystal violet | antifungal |
| echinocandin | antifungal |
| econazole | antifungal |
| efinaconazole | antifungal |
| epoxiconazole | antifungal |
| fenticonazole | antifungal |
| filipin | antifungal |
| fluconazole | antifungal |
| flucytosine | antifungal |
| griseofulvin | antifungal |
| haloprogin | antifungal |
| hamycin | antifungal |
| imidazole | antifungal |
| isavuconazole | antifungal |
| isoconazole | antifungal |
| itraconazole | antifungal |
| ketoconazole | antifungal |
| luliconazole | antifungal |
| micafungin | antifungal |
| miconazole | antifungal |
| miltefosine | antifungal |
| naftitine | antifungal |
| natamycin | antifungal |
| nystatin | antifungal |
| omoconazole | antifungal |
| orotomide | antifungal |
| oxiconazole | antifungal |
| polyene antifungal | antifungal |
| posaconazole | antifungal |
| propiconazole | antifungal |
| ravuconazole | antifungal |
| rimocidin | antifungal |
| sertaconazole | antifungal |
| sulconazole | antifungal |
| terbinafine | antifungal |
| terconazole | antifungal |
| thiazole | antifungal |
| tioconazole | antifungal |
| tolnaftate | antifungal |
| triazole | antifungal |
| undecylenic acid | antifungal |
| voriconazole | antifungal |
| bacterial cell | biologic |
| microbiome | biologic |
| microorganism | biologic |
| rhizobia bacteria | biologic |
| symbiote | biologic |
| virus | biologic |
| virus fragment | biologic |
| virus particle | biologic |
| fungicide | biologic; pesticide |
| Pour-Ons | ectoparasiticide |
| Sprays | ectoparasiticide |
| Dips | ectoparasiticide |
| Ear Tags | ectoparasiticide |
| Collars | ectoparasiticide |
| Oral Tablets | ectoparasiticide |
| Other Ectoparasiticides | ectoparasiticide |
| Spot-Ons | ectoparasiticide |
| ectoparaciticide | ectoparasiticide |
| pyrethroid | ectoparasiticide |
| carbamate | ectoparasiticide |
| water-insoluble organo-phospphorus | ectoparasiticide |
| compound | |
| benzoyl urea | ectoparasiticide |
| formamidine | ectoparasiticide |
| triazine | ectoparasiticide |
| avermectin | ectoparasiticide |
| milbemycin | ectoparasiticide |
| flumethrin | ectoparasiticide |
| alphamethrin | ectoparasiticide |
| pirimphos methyl | ectoparasiticide |
| pirimphos ethyl | ectoparasiticide |
| mylbemycin | ectoparasiticide |
| moxidectin | ectoparasiticide; endoparasiticide |
| ivermectin | ectoparasiticide; endoparasiticide; |
| | insecticide |
| doramectin | ectoparasiticide; endoparasiticide; |
| | insecticide |
| abamectin | ectoparasiticide; insecticide |
| pyrethrin | ectoparasiticide; insecticide |
| cyhalothrin | ectoparasiticide; insecticide |
| amitraz | ectoparasiticide; insecticide |
| deltamethrin | ectoparasiticide; insecticide |
| diazinon | ectoparasiticide; insecticide |
| macrocyclic lactones | endecticide |
| benzimidazole | endecticide |
| pro-benzimidazole | endecticide |
| imidazothiazole | endecticide |
| tetrahydropyrimidine | endecticide |
| organophosphate | endecticide |
| Endoparasiticide | endoparasiticide |
| Oral Liquids | endoparasiticide |
| Oral Solids | endoparasiticide |
| Injectables | endoparasiticide |
| Feed Additives | endoparasiticide |
| Endectocides | endoparasiticide |
| Tetramisole | endoparasiticide |
| dexamisole | endoparasiticide |
| Milbemycin oxime | endoparasiticide |
| Nemadectin | endoparasiticide |
| Albendazole | endoparasiticide |
| Clorsulon | endoparasiticide |
| Cydectin | endoparasiticide |
| Diethylcarbamazine | endoparasiticide |
| Febantel | endoparasiticide |
| Fenbendazole | endoparasiticide |
| Haloxon | endoparasiticide |
| Levamisole | endoparasiticide |
| Mebendazole | endoparasiticide |
| Morantel | endoparasiticide |
| Oxyclozanide | endoparasiticide |
| Oxibendazole | endoparasiticide |
| Oxfendazole | endoparasiticide |
| Oxamniquine | endoparasiticide |
| Pyrantel | endoparasiticide |
| Praziquantel | endoparasiticide |
| Thiabendazole | endoparasiticide |
| cyclosporin | endoparasiticide |
| sulfonamide | endoparasiticide |
| cephalosporin | endoparasiticide |
| cephamycin | endoparasiticide |
| aminoglucosid | endoparasiticide |
| trimethoprim | endoparasiticide |
| dimetridazole | endoparasiticide |
| framycetin | endoparasiticide |
| fruazolidone | endoparasiticide |
| pleuromutilin | endoparasiticide |
| a compound active against | endoparasiticide |
| protozoa | |
| piperazine | endoparasiticide; endecticide |
| emamectin | endoparasiticide; insecticide |
| eprinomectin | endoparasiticide; insecticide |
| milbemectin | endoparasiticide; insecticide |
| permethrin | endoparasiticide; insecticide |
| selamectin | endoparasiticide; insecticide |
| trichlorfon | endoparasiticide; insecticide |
| ammonium nitrate | fertilizer |
| binary fertilizer | fertilizer |
| compound fertilizer | fertilizer |
| diammonium phosphate | fertilizer |
| fertilizer | fertilizer |
| monoammonium phosphate | fertilizer |
| multinutrient fertilizer | fertilizer |
| natural fertilizer | fertilizer |
| nitrogen fertilizer | fertilizer |
| NK fertilizer | fertilizer |
| NP fertilizer | fertilizer |
| NPK fertilizer | fertilizer |
| organic fertilizer | fertilizer |
| phosphate fertilizer | fertilizer |
| PK fertilizer | fertilizer |
| potassium fertilizer | fertilizer |
| single-nutrient fertilizer | fertilizer |
| superphosphate | fertilizer |
| synthetic fertilizer | fertilizer |
| urea | fertilizer |
| binapacryl | fungicide |
| Bisphenol A | fungicide |
| copper 8-hydroxyquinoline | fungicide |
| copper sulfate | fungicide |
| mercuric chloride | fungicide |
| phenol | fungicide |
| phenylmercuric oleate | fungicide |
| tributyltin chloride | fungicide |
| tributyltin triacetate | fungicide |
| pentachlorophenol | fungicide; insecticide |
| Bupirimate | fungicide; pesticide |
| Captan | fungicide; pesticide |
| Carbendazim | fungicide; pesticide |
| Chloranil | fungicide; pesticide |
| antibiotic | general |
| fertilizer | general |
| nutrient | general |
| pH modulator | general |
| small molecule | general |
| soil stabilizer | general |
| therapeutic agent | general |
| 2,4,5-T | herbicide |
| 2,4-D | herbicide |
| atrazine | herbicide |
| chlorophenoxy acid | herbicide |
| cynazine | herbicide |
| glyphosate | herbicide |
| hexazinone | herbicide |
| MCPA | herbicide |
| metribuzin | herbicide |
| organic phosphorus herbicide | herbicide |
| silvex | herbicide |
| simazine | herbicide |
| triazine herbicide | herbicide |
| bromacil | herbicide; pesticide |
| Chloramben | herbicide; pesticide |
| Chlorfenac | herbicide; pesticide |
| Chlorsulfuron | herbicide; pesticide |
| Abscisic acid | hormone |
| Auxins | hormone |
| Cytokinins | hormone |
| Ethylene | hormone |
| Gibberellins | hormone |
| steroid | hormone |
| dexamethasone | hormone |
| allopregnanolone | hormone |
| estrogen | hormone |
| ethinylestradiol | hormone |
| mestranol | hormone |
| estradiols | hormone |
| estriol | hormone |
| estriolsuccinate | hormone |
| polyestriolphosphate | hormone |
| estrone | hormone |
| estronesulfate | hormone |
| conjugatedestrogens | hormone |
| progesterone | hormone |
| norethisteroneacetate | hormone |
| norgestrel | hormone |
| levonorgestrel | hormone |
| gestodene | hormone |
| chlormadinoneacetate | hormone |
| drospirorenone | hormone |
| 3-ketodesogestrel | hormone |
| androgen | hormone |
| testosterone | hormone |
| androstenediol | hormone |
| androstenedione | hormone |
| dehydroepiandrosterone | hormone |
| dihydrotestosterone | hormone |
| anymineralocorticoid | hormone |
| anyglucocoriticoid | hormone |
| cholesterols | hormone |
| 1,2-dichloropropane | insecticide |
| acephate | insecticide |
| acetamiprid | insecticide |
| acethion | insecticide |
| acetoprole | insecticide |
| acrinathrin | insecticide |
| acrylonitrile | insecticide |
| alanycarb | insecticide |
| aldicarb | insecticide |
| aldnrn | insecticide |
| aldoxycarb | insecticide |
| allethrin | insecticide |
| allosamidin | insecticide |
| allyxycarb | insecticide |
| alpha-cypermethrin | insecticide |
| amidithion | insecticide |
| aminocarb | insecticide |
| amiton | insecticide |
| anabasine | insecticide |
| athidathion | insecticide |
| azadirachtin | insecticide |
| azamethiphos | insecticide |
| azinphos-ethyl | insecticide |
| azinphos-methyl | insecticide |
| azothoate | insecticide |
| barium hexafluorosilicate | insecticide |
| barthrin | insecticide |
| bendiocarb | insecticide |
| benfuracarb | insecticide |
| bensultap | insecticide |
| beta-cyfluthrin | insecticide |
| beta-cypermethrin | insecticide |
| bifenthrin | insecticide |
| bioallethrin | insecticide |
| bioethanomethrin | insecticide |
| biopermethrin | insecticide |
| bioresmethrin | insecticide |
| bistrifluron | insecticide |
| borax | insecticide |
| botanical insecticide | insecticide |
| bromfenvinfos | insecticide |
| bromo-DDT | insecticide |
| bromophos-ethyl | insecticide |
| bufencarb | insecticide |
| buprofezin | insecticide |
| butacarb | insecticide |
| butathiofos | insecticide |
| butocarboxim | insecticide |
| butonate | insecticide |
| butoxycarboxim | insecticide |
| cadusafos | insecticide |
| calcium arsenate | insecticide |
| calcium polysulfide | insecticide |
| camphechlor | insecticide |
| carbanolate | insecticide |
| carbofuran | insecticide |
| carbon disulfide | insecticide |
| carbon tetrachloride | insecticide |
| carbosulfan | insecticide |
| cartap | insecticide |
| chlorbicyclen | insecticide |
| chlordane | insecticide |
| chlordecone | insecticide |
| chlorethoxyfos | insecticide |
| chlorfenapyr | insecticide |
| chlorfenvinphos | insecticide |
| chlorfluazuron | insecticide |
| chlormephos | insecticide |
| chloroform | insecticide |
| chloropicrin | insecticide |
| chlorphoxim | insecticide |
| chlorprazophos | insecticide |
| chlorpyrifos-methyl | insecticide |
| chlorthiophos | insecticide |
| chromafenozide | insecticide |
| cinerin I | insecticide |
| cinerin II | insecticide |
| cismethrin | insecticide |
| cloethocarb | insecticide |
| closantel | insecticide |
| clothianidin | insecticide |
| copper acetoarsenite | insecticide |
| copper arsenate | insecticide |
| coumaphos | insecticide |
| coumithoate | insecticide |
| crotamiton | insecticide |
| crotoxyphos | insecticide |
| crufomate | insecticide |
| cryolite | insecticide |
| cyanofenphos | insecticide |
| cyanophos | insecticide |
| cyanthoate | insecticide |
| cyclethrin | insecticide |
| cycloprothrin | insecticide |
| cyfluthrin | insecticide |
| cypermethrin | insecticide |
| cyphenothrin | insecticide |
| cyromazine | insecticide |
| cythioate | insecticide |
| DDT | insecticide |
| decarbofuran | insecticide |
| demephion | insecticide |
| demephion-O | insecticide |
| demephion-S | insecticide |
| demeton | insecticide |
| demeton-methyl | insecticide |
| demeton-O | insecticide |
| demeton-O-methyl | insecticide |
| demeton-S | insecticide |
| demeton-S-methyl | insecticide |
| demeton-S-methylsulphon | insecticide |
| diafenthiuron | insecticide |
| dialifos | insecticide |
| dicapthon | insecticide |
| dichlofenthion | insecticide |
| dichlorvos | insecticide |
| dicresyl | insecticide |
| dicrotophos | insecticide |
| dicyclanil | insecticide |
| dieldrin | insecticide |
| diflubenzuron | insecticide |
| dilor | insecticide |
| dimefox | insecticide |
| dimetan | insecticide |
| dimethoate | insecticide |
| dimethrin | insecticide |
| dimethylvinphos | insecticide |
| dimetilan | insecticide |
| dinex | insecticide |
| dinoprop | insecticide |
| dinosam | insecticide |
| dinotefuran | insecticide |
| diofenolan | insecticide |
| dioxabenzofos | insecticide |
| dioxacarb | insecticide |
| dioxathion | insecticide |
| disulfoton | insecticide |
| dithicrofos | insecticide |
| d-limonene | insecticide |
| ecdysterone | insecticide |
| empenthrin | insecticide |
| endosulfan | insecticide |
| endothion | insecticide |
| endrin | insecticide |
| epofenonane | insecticide |
| esfenvalerate | insecticide |
| etaphos | insecticide |
| ethiofencarb | insecticide |
| ethion | insecticide |
| ethiprole | insecticide |
| ethoate-methyl | insecticide |
| ethoprophos | insecticide |
| ethyl formate | insecticide |
| ethylene dibromide | insecticide |
| ethylene dichloride | insecticide |
| ethylene oxide | insecticide |
| etofenprox | insecticide |
| etrimfos | insecticide |
| famphur | insecticide |
| fenamiphos | insecticide |
| fenazaflor | insecticide |
| fenchlorphos | insecticide |
| fenethacarb | insecticide |
| fenfluthrin | insecticide |
| fenitrothion | insecticide |
| fenobucarb | insecticide |
| fenoxacrim | insecticide |
| fenoxycarb | insecticide |
| fenpirithrin | insecticide |
| fenpropathrin | insecticide |
| fensulfothion | insecticide |
| fenthion | insecticide |
| fenthion-ethyl | insecticide |
| fenvalerate | insecticide |
| fipronil | insecticide |
| flonicamid | insecticide |
| flucofuron | insecticide |
| flucycloxuron | insecticide |
| flucythrinate | insecticide |
| flufenerim | insecticide |
| flufenoxuron | insecticide |
| flufenprox | insecticide |
| fluvalinate | insecticide |
| fonofos | insecticide |
| formetanate | insecticide |
| formothion | insecticide |
| formparanate | insecticide |
| fosmethilan | insecticide |
| fospirate | insecticide |
| fosthietan | insecticide |
| furathiocarb | insecticide |
| furethrin | insecticide |
| gamma-cyhalothrin | insecticide |
| halfenprox | insecticide |
| halofenozide | insecticide |
| heptachlor | insecticide |
| heptenophos | insecticide |
| heterophos | insecticide |
| hexaflumuron | insecticide |
| hydramethylnon | insecticide |
| hydrogen cyanide | insecticide |
| hydroprene | insecticide |
| hyquincarb | insecticide |
| imidacloprid | insecticide |
| imiprothrin | insecticide |
| indoxacarb | insecticide |
| isazofos | insecticide |
| isobenzan | insecticide |
| isodrin | insecticide |
| isofenphos | insecticide |
| isoprocarb | insecticide |
| isoprothiolane | insecticide |
| isothioate | insecticide |
| isoxathion | insecticide |
| isoxazole | insecticide |
| jasmolin I | insecticide |
| jasmolin II | insecticide |
| jodfenphos | insecticide |
| juvenile hormone I | insecticide |
| juvenile hormone II | insecticide |
| juvenile hormone III | insecticide |
| kelevan | insecticide |
| kinoprene | insecticide |
| lambda-cyhalothrin | insecticide |
| lead arsenate | insecticide |
| leptophos | insecticide |
| lirimfos | insecticide |
| lufenuron | insecticide |
| lythidathion | insecticide |
| malathion | insecticide |
| malonoben | insecticide |
| mazidox | insecticide |
| mecarbam | insecticide |
| mecarphon | insecticide |
| menazon | insecticide |
| mephosfolan | insecticide |
| mercurous chloride | insecticide |
| mesulfenfos | insecticide |
| methacrifos | insecticide |
| methamidophos | insecticide |
| methidathion | insecticide |
| methiocarb | insecticide |
| methocrotophos | insecticide |
| methomyl | insecticide |
| methoprene | insecticide |
| methoxychlor | insecticide |
| methoxyfenozide | insecticide |
| methyl bromide | insecticide |
| methylchloroform | insecticide |
| methylene chloride | insecticide |
| metofluthrin | insecticide |
| metolcarb | insecticide |
| metoxadiazone | insecticide |
| mevinphos | insecticide |
| mexacarbate | insecticide |
| mipafox | insecticide |
| mirex | insecticide |
| monocrotophos | insecticide |
| morphothion | insecticide |
| naftalofos | insecticide |
| naled | insecticide |
| naphthalene | insecticide |
| nicotine | insecticide |
| nifluridide | insecticide |
| nitenpyram | insecticide |
| nithiazine | insecticide |
| nitrilacarb | insecticide |
| novaluron | insecticide |
| noviflumuron | insecticide |
| omethoate | insecticide |
| oxamyl | insecticide |
| oxydemeton-methyl | insecticide |
| oxydeprofos | insecticide |
| oxydisulfoton | insecticide |
| para-dichlorobenzene | insecticide |
| parathion | insecticide |
| parathion-methyl | insecticide |
| penfluron | insecticide |
| phenkapton | insecticide |
| phenothrin | insecticide |
| phenthoate | insecticide |
| phorate | insecticide |
| phosalone | insecticide |
| phosfolan pirimetaphos | insecticide |
| phosmet | insecticide |
| phosnichlor | insecticide |
| phosphamidon | insecticide |
| phosphine | insecticide |
| phoxim | insecticide |
| phoxim-methyl | insecticide |
| pirimicarb | insecticide |
| pirimiphos-ethyl | insecticide |
| pirimiphos-methyl | insecticide |
| potassium arsenite | insecticide |
| potassium thiocyanate | insecticide |
| pp′-DDT | insecticide |
| prallethrin | insecticide |
| precocene I | insecticide |
| precocene II | insecticide |
| precocene III | insecticide |
| primidophos | insecticide |
| profenofos | insecticide |
| profluthrin | insecticide |
| promacyl | insecticide |
| promecarb | insecticide |
| propaphos | insecticide |
| propetamphos | insecticide |
| propoxur | insecticide |
| prothidathion | insecticide |
| prothiofos | insecticide |
| prothoate | insecticide |
| protrifenbute | insecticide |
| pyraclofos | insecticide |
| pyrazophos | insecticide |
| pyresmethrin | insecticide |
| pyrethrin I | insecticide |
| pyrethrin II | insecticide |
| pyridaben | insecticide |
| pyridalyl | insecticide |
| pyridaphenthion | insecticide |
| pyrimidifen | insecticide |
| pyrimitate | insecticide |
| pyriproxyfen | insecticide |
| quassia | insecticide |
| quinalphos | insecticide |
| quinalphos-methyl | insecticide |
| quinothion | insecticide |
| rafoxanide | insecticide |
| resmethrin | insecticide |
| rotenone | insecticide |
| ryania | insecticide |
| sabadilla | insecticide |
| schradan | insecticide |
| silafluofen | insecticide |
| sodium arsenite | insecticide |
| sodium fluoride | insecticide |
| sodium hexafluorosilicate | insecticide |
| sodium thiocyanate | insecticide |
| sophamide | insecticide |
| spinosad | insecticide |
| spiromesifen | insecticide |
| sulcofuron | insecticide |
| sulfluramid | insecticide |
| sulfotep | insecticide |
| sulfuryl fluoride | insecticide |
| sulprofos | insecticide |
| tau-fluvalinate | insecticide |
| tazimcarb | insecticide |
| tebufenozide | insecticide |
| tebufenpyrad | insecticide |
| tebupirimfos | insecticide |
| teflubenzuron | insecticide |
| tefluthrin | insecticide |
| temephos | insecticide |
| terallethrin | insecticide |
| terbufos | insecticide |
| tetrachloroethane | insecticide |
| tetrachlorvinphos | insecticide |
| tetramethrin | insecticide |
| theta-cypermethrin | insecticide |
| thiacloprid | insecticide |
| thiamethoxam | insecticide |
| thicrofos | insecticide |
| thiocarboxime | insecticide |
| tbiocyclam | insecticide |
| thiodicarb | insecticide |
| thiofanox | insecticide |
| thiometon | insecticide |
| thiosultap | insecticide |
| thuringiensin | insecticide |
| tolfenpyrad | insecticide |
| tralomethrin | insecticide |
| transfluthrin | insecticide |
| transpermethrin | insecticide |
| triarathene | insecticide |
| triazamate | insecticide |
| triazophos | insecticide |
| trichlormetaphos-3 | insecticide |
| trichloronat | insecticide |
| trifenofos | insecticide |
| triflumuron | insecticide |
| trimethacarb | insecticide |
| triprene | insecticide |
| vamidothion | insecticide |
| vaniliprole | insecticide |
| xylylcarb | insecticide |
| zeta-cypermethrin | insecticide |
| zolaprofos | insecticide |
| α-ecdysone | insecticide |
| bromophos | insecticide; pesticide |
| chlordimeform | insecticide; pesticide |
| Carbaryl | insecticide; pesticide |
| Carbophenothion | insecticide; pesticide |
| Chlorpyrifos | insecticide; pesticide |
| amino acid | macronutrient |
| amylopectin | macronutrient |
| amylose | macronutrient |
| arachidic acid | macronutrient |
| behenic acid | macronutrient |
| butyric acid | macronutrient |
| capric acid | macronutrient |
| caprioic acid | macronutrient |
| caprylic acid | macronutrient |
| carbohydrate | macronutrient |
| cerotic acid | macronutrient |
| cervonic acid | macronutrient |
| clupanodonic acid | macronutrient |
| eicosen | macronutrient |
| erucic acid | macronutrient |
| essential fatty acid | macronutrient |
| fat | macronutrient |
| fructose | macronutrient |
| galactose | macronutrient |
| glucose | macronutrient |
| heptadecanoic acid | macronutrient |
| lactose | macronutrient |
| lauric acid | macronutrient |
| lignoceric acid | macronutrient |
| linoleic acid | macronutrient |
| Macronutrient | macronutrient |
| maltose | macronutrient |
| margaric acid | macronutrient |
| monounsaturated fat | macronutrient |
| myristic acid | macronutrient |
| myristol | macronutrient |
| nervonic acid | macronutrient |
| oleic acid | macronutrient |
| palmitic acid | macronutrient |
| palmitoyl | macronutrient |
| pentadecanoic acid | macronutrient |
| polyunsaturated fat | macronutrient |
| protein | macronutrient |
| ribose | macronutrient |
| saturated fat | macronutrient |
| stearic acid | macronutrient |
| steridonic acid | macronutrient |
| sucrose | macronutrient |
| timnodonic acid | macronutrient |
| α-linoleic acid | macronutrient |
| calcium | micronutrient |
| chloride | micronutrient |
| chromium | micronutrient |
| copper | micronutrient |
| iodine | micronutrient |
| iron | micronutrient |
| magnesium | micronutrient |
| manganese | micronutrient |
| mineral | micronutrient |
| molybdenum | micronutrient |
| nickel | micronutrient |
| phosphorus | micronutrient |
| potassium | micronutrient |
| selenium | micronutrient |
| silicon | micronutrient |
| tin | micronutrient |
| vanadium | micronutrient |
| vitamin | micronutrient |
| vitamin A | micronutrient |
| vitamin B-1 | micronutrient |
| vitamin B-12 | micronutrient |
| vitamin B-2 | micronutrient |
| vitamin B-3 | micronutrient |
| vitamin B-5 | micronutrient |
| vitamin B-6 | micronutrient |
| vitamin B-7 | micronutrient |
| vitamin B-9 | micronutrient |
| vitamin C | micronutrient |
| vitamin D | micronutrient |
| vitamin E | micronutrient |
| vitamin K | micronutrient |
| zinc | micronutrient |
| adhesive | pest control agent |
| allomone | pest control agent |
| anti-disease agent | pest control agent |
| antifeedant | pest control agent |
| antifungal | pest control agent |
| behavior-modifying compound | pest control agent |
| bird repellent | pest control agent |
| black pepper | pest control agent |
| caffeine | pest control agent |
| capsaicin | pest control agent |
| capsaicin oleoresin | pest control agent |
| catnip oil | pest control agent |
| chemosterilant | pest control agent |
| chili powder | pest control agent |
| complex sugar | pest control agent |
| dill | pest control agent |
| ginger | pest control agent |
| gum | pest control agent |
| herbicide | pest control agent |
| insect attractant | pest control agent |
| insect repellent | pest control agent |
| insecticide | pest control agent |
| kairomone | pest control agent |
| mammal repellent | pest control agent |
| mating disrupter | pest control agent |
| monoterpenoid | pest control agent |
| paprika | pest control agent |
| pest control agent | pest control agent |
| pesticide | pest control agent |
| phenolic compound | pest control agent |
| pheromone | pest control agent |
| red pepper | pest control agent |
| acaricide | pesticide |
| algicide | pesticide |
| avicide | pesticide |
| Bacillus thuringiensis | pesticide |
| endotoxin polypeptide | |
| bactericide | pesticide |
| Bis(p-chlorophenoxy)methane | pesticide |
| Bitertanol | pesticide |
| Bromadiolone | pesticide |
| Bromethalinlin | pesticide |
| Bromopropylate | pesticide |
| Busulfan | pesticide |
| Butrylin | pesticide |
| Cambendazole | pesticide |
| Candicidin | pesticide |
| Candidin | pesticide |
| Chloramphenacol | pesticide |
| Chlorbetamide | pesticide |
| Chlorothion | pesticide |
| Chlorphenesin | pesticide |
| molluscicide | pesticide |
| nematicide | pesticide |
| rodenticide | pesticide |
| virucide | pesticide |
| biopolymer | soil stabilizer |
| chemical | soil stabilizer |
| co-polymer | soil stabilizer |
| enzyme | soil stabilizer |
| fiber reinforcement agent | soil stabilizer |
| flowability agent | soil stabilizer |
| hydrophilic agent | soil stabilizer |
| hydrophobic agent | soil stabilizer |
| ionic stabilizer | soil stabilizer |
| polymer | soil stabilizer |
| resin | soil stabilizer |
| salt | soil stabilizer |
| surfactant | soil stabilizer |
| chelated micronutrient | therapeutic agent |
| microbe | therapeutic agent |
| non-chelated micronutrient | therapeutic agent |
| probiotic | therapeutic agent |

In one embodiment, the cargo for use in SBP formulations may be hormone analogue such as, but not limited to, Deslorelin.

### Coating

In some embodiments, SBP agricultural compositions may include one or more coatings. As used herein, the term “coating” refers to any substance that is applied to the surface of another substance. In some embodiments, the coating may be functional, decorative or both. Coatings may be applied to completely cover the surface. Coating may also be applied to partially cover the surface. In some embodiments, coatings may include processed silk. In some aspects, the coating may be SBP. Coatings may also include but are not limited to any of the cargos described in Table 7.

In some embodiment, the coating may be a seed coating. SBPs described herein may provide important properties necessary for the safe and effective delivery of the cargo that are beneficial to the health and development of a seed. In some embodiments, the coating may be a leaf coating. In some embodiments, agricultural compositions described herein, such as coatings, may be able to penetrate plants, leaves, seeds, roots, and/or any other part of the plant described herein. In some aspects, the SBP may be useful in protection of the roots, increasing the availability of nutrients, enhancing growth of the plant, increasing resistance of the plant to disease, deterring pathogens and pests, and increasing resistance of the plant to environmental conditions such as heat, flooding, and drought. These properties and advantages of the SBPs described herein will offer safe alternatives to current matrices used for seed coatings and will allow increased tailoring of seed coatings according to seed type, soil characteristics, regional climate, local pathogens, pests, and application equipment.

In some embodiment, the coating may be a plant coating. SBP coatings may incorporate one or more cargos that are beneficial to the health and development of the plant. SBP coatings may incorporate therapeutic agents for the treatment of plant diseases. In some embodiments, the cargo may include but is not limited to any of the cargos described in Table 7. In some embodiments, the coating covers the whole plant. In some embodiments, the coating covers a part of the plant (non-limiting examples include leaf, pollen, embryo, root, root tip, anther, flower, seed, vegetable, leave, xylem, phloem, stems, fruits, fruiting body, and propagules). Any SBP format described in the present disclosure may be used to prepare plant coating formulations. In some embodiments, the plant coating formulations are hydrogels. In some embodiments, the SBP coating has a residence time of days to months.

In some embodiments, SBP coatings may be applied to seeds and/or plants to stabilize, maintain, or promote the growth of the microbes, microorganisms, and/or microbiomes inhabiting on the surface. In some embodiments, SBP compositions used for seed and/or plant coating may incorporate beneficial microbes, microorganisms, and/or microbiomes, such as any of those described herein. It has been shown that certain bacteria (e.g., Rhizobium) added to the seeds could boost crop production. Seed coating formulations incorporating microbial compositions have been described, for example, in US Publication Number US20140342905, the contents of which are incorporated by reference in their entirety. Any SBP format described in the present disclosure may be used to prepare seed coating formulations. In some embodiments, the seed coating formulations are hydrogels. In some embodiments, the SBP coating has a residence time of days to months.

In some embodiments, the SBP coating may be used for one or more applications, including, but not limited to, protection of a seed, plant, planting substrate, agricultural product, or device; fertilizing and/or promoting germination of a coated seed or plant; encasing a payload; delivering a payload, modulating nutrient and/or water uptake; stabilizing a payload; and/or controlling the release of a payload.

In some embodiments, SBP coatings may be applied to a fruit or a vegetable to prevent spoilage. It is estimated that about a quarter of harvested fruit and vegetables are lost due to microbial spoilage during storage and transport. Silk fibroin coatings have been shown to enhance fruits' shelf-life at room conditions by reducing cell respiration rate and water evaporation (Marelli et al. (2016) Scientific Reports 6:25263, the contents of which are hereby incorporated by reference in their entirety). Additionally, silk fibroin coatings are edible, flavorless and odorless, which are compelling properties for food coating. In some embodiments, the SBP coating may be applied a climacteric fruit. Climacteric fruits ripen through ethylene production and increased cell respiration. Such fruits include, but are not limited to, apple, banana, mango, papaya, pear, apricot, peach, plum, avocado, plantain, guava, nectarine, passion fruit, blueberry, cantaloupe, and tomato. In some embodiments, the SBP coating may be applied a non-climacteric fruit. On the contrary, non-climacteric fruits ripen without ethylene and respiration bursts. Such fruits include, but are not limited to, orange, mousambi, kinnow, grapefruit, grapes, pomegranate, litchi, watermelon, cherry, raspberry, blackberry, strawberry, carambola, rambutan, and cashew.

### Fertilizer

In some embodiments, the SBP agricultural compositions of the present disclosure may include fertilizers. As used herein, the term “fertilizer” refers to any substance, natural or artificial that may be used to improve growth and/or yield of plants. The fertilizer may be applied directly to the plant or a portion of the plant, or it may be applied to the locus i.e. the substrate on which the plant grows or is expected to grow. In some embodiments, the fertilizer may be SBPs, processed silk and/or processed silk preparations. The fertilizers may be natural fertilizers, synthetic fertilizers, or a combination thereof. In some embodiments, the fertilizers are single-nutrient fertilizers (e.g. ammonium nitrate, superphosphates, and urea), binary fertilizers (e.g. NP fertilizers, NK fertilizers, PK fertilizers, monoammonium phosphate, diammonium phosphate), multinutrient fertilizers (NPK fertilizers), nitrogen fertilizers, phosphate fertilizers, potassium fertilizers, compound fertilizers, and organic fertilizers. SBPs offer an eco-friendly alternative to many synthetic chemicals used in fertilizers because SBPs are biocompatible and biodegradable.

In some embodiments, SBP agricultural compositions may encapsulate fertilizers for extended and/or controlled release. Slow release of the nutrients from fertilizers is beneficial to building a healthy soil environment and decreasing the hazard of runoff into nearby lakes and streams. Extended release may also prevent over-fertilizing or “fertilizer burn” of the plants or seeds.

### Nutrient

In some embodiments, the SBP agricultural compositions may include a nutrient. These nutrients may be macronutrients and micronutrients. Macronutrients that may be used in the agricultural compositions include, but are not limited to, carbohydrates (e.g. fructose, glucose, sucrose, ribose, amylose, amylopectin, maltose, lactose, and galactose), proteins, amino acids, fats, saturated fats (e.g. butyric acid, caprioic acid, caprylic acid, capric acid, lauric acid, myristic acid, pentadecanoic acid, palmitic acid, margaric acid, stearic acid, arachidic acid, behenic acid, lignoceric acid, and cerotic acid), monounsaturated fats (e.g. myristol, pentadecanoic acid, palmitoyl, heptadecanoic acid, oleic acid, eicosen, erucic acid, and nervonic acid), polyunsaturated fats (e.g. steridonic acid, arachidic acid, timnodonic acid, clupanodonic acid, and cervotic acid), and essential fatty acids (e.g. linoleic acid and α-linoleic acid). Micronutrients that may be used as payloads include, but are not limited to, vitamins (e.g. vitamin A, vitamin B-1, vitamin B-2, vitamin B-3, vitamin B-5, vitamin B-6, vitamin B-7, vitamin B-9, vitamin B-12, vitamin C, vitamin D, vitamin E, and vitamin K) and minerals (e.g. calcium, iron, phosphorus, iodine, magnesium, zinc, selenium, selenium, copper, manganese, chromium, molybdenum, chloride, potassium, nickel, silicon, vanadium, and tin).

In some embodiments, the SBP agricultural compositions may include essential nutrients that are beneficial to the health and development of agricultural products. There are at least 17 micronutrients that are critical to the optimal germination, growth, and development of seeds. Various approaches have been utilized in order to ensure that seeds are supplied with adequate concentrations of these micronutrients. These include seed coatings with compositions that include a micronutrient, or seed priming. Various formats of SBPs may be utilized in order to deliver combinations of micronutrients to a germinating and developing seed. In the case of seed priming, the seed may be first partially hydrated under controlled conditions that supply the required micronutrient concentrations, with the seed then redried prior to planting. In some embodiment, the essential micronutrient can be any of the essential micronutrients known in the art.

In some embodiments, nutrients for use in SBPs may be selected from any of those listed in Table 7, above.

### Agricultural Products

In some embodiments, SBP agricultural compositions may include one or more agricultural products. These agricultural products may be plants, animals, plant agricultural products, and animal agricultural products.

In some embodiments, SBP agricultural compositions may include plants. The methods and SBPs of the present disclosure may have applications in plants. In some embodiments, SBPs will serve as agricultural composition to facilitate the production of plants. In some embodiments the plants are agricultural plants i.e., plants for farming purposes. In some embodiments, the plants are silvicultural plants, i.e. plants for the controlling the growth, health, establishment, composition, and quality of forests. In some embodiments, the plants are ornamental plants. In some embodiments, the plants are edible plants. In some embodiments, the plants are horticultural plants. In some embodiments, the plants are natural or wild-type plants. In other embodiments, the plants are genetically modified plants. In some aspects, the plants are medicinal plants.

In some embodiments, the plants used with SBP agricultural compositions of the present disclosure may be monocots. In some embodiments, the plants used with the agricultural compositions of the present disclosure may be dicots. In some embodiments, the plants used with the agricultural compositions of the present disclosure may be gymnosperms. In some embodiments, the plants used with the agricultural compositions of the present disclosure may be angiosperms. Non-limiting examples of plants include acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, hops, kale, kiwifruit, kohlrabi, larch, lettuce, leek, lemon, lime, locust, pine, maidenhair, maize, mango, maple, marijuana, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. In some embodiments, the plants used with the agricultural compositions of the present disclosure may also encompass algae, which are mainly photoautotrophs unified primarily by their lack of roots, leaves and other organs that characterize higher plants.

In some embodiments, the agricultural products may be portions of plants. These portions of the plant include, but are not limited to, leaf, pollen, embryo, root, root tip, anther, flower, seed, vegetable, leave, xylem, phloem, stems, fruits, fruiting body, and propagules (e.g. cuttings).

In some embodiments, agricultural products may include animals and/or animal agricultural products. In some embodiments, the animals used with agricultural compositions of the present disclosure include but are not limited to cows, bulls, sheep, goat, bison, turkey, buffalo, pigs, poultry, horses, alpaca, llama, camels, rabbits, guinea pigs, fish, shrimps, crustaceans, mollusks, insects, silk worms, bees, and crickets. In some aspects, the animals used with SBP agricultural compositions may be any of the non-human animals listed in Table 2, above.

In some embodiments, the SBP agricultural compositions may be or may include one or more animal agricultural products. Animal agricultural products may include, but are not limited to milk, butter, cheese, yogurt, whey, curds, meat, oil, fat, blood, amino acids, hormones, enzymes, wax, feathers, fur, hide, bones, gelatin, horns, ivory, wool, venom, tallow, silk, sponges, manure, eggs, pearl culture, honey, and food dye. In some embodiments, the animal agricultural product is a dairy product. Non-limiting examples of dairy products include milk, cream, cheese, clotted cream, sour cream, gelato, ghee, infant formula, powdered milk, butter, crème fraiche, ice cream, yoghurt, curds, whey, custard, dulce de leche, evaporated milk, eggnog, frozen yoghurt, frozen custard, buttermilk, formula, casein, condensed milk, cottage cheese, and cream cheese.

### Pest Control Agent

In some embodiments, SBP agricultural compositions may include pest control agents. In some aspects, the SBPs may be a pest control agent. As used herein, the term “pest” refers to any organism that harms, irritates, causes discomfort, or generally annoys another organism. Pests may include, but are not limited to, non-human animals, insects, spiders, ticks, fleas, parasites, worms, plants, algae, microbes, microorganisms, fungi, bacteria, yeast, and viruses. Non-limiting examples of pests include, mice, rats, squirrels, rodents, opossums, pigeons, seagulls, crows, geese, woodpeckers, the common myna, raccoons, bears, bats, beavers, voles, rabbits, deer, coyotes, wolves, squirrels, boars, elk, birds, foxes, gophers, moles and household pets. Other non-limiting examples of pests include red spider mites, gall mites, leaf miners, moths, flies, moths, sawflies, beetles, box suckers, nematodes, codling moths, winter moths, scale insects, whiteflies, viburnum beetles, thrips, vine weevils, caterpillars, cabbage white caterpillars, tomato moths, aphids, wooly beech aphids, earwigs, fleas, ticks, mosquitos, boll weevils, weeds, frogs, toads, phylloxera, Lepidopteran larvae, Dipteran larvae, Coleopteran larvae, locusts, crickets, ants, cockroaches, flies, wasps, termites, woodworms, wood ants, bookworms, silverfish, carpet beetles, Japanese beetles. Africanized bees, Colorado potato beetles, western root cornworms, clothes moths, gypsy moths, any ectoparasite (e.g. chiggers, mites, ticks, lice, fleas, bedbugs, mosquitos, tsetse flies, and kissing bugs), any gastropod mollusk (e.g. slugs and snails), and any invasive species. SBPs used for agricultural applications related to pest control may be used to kill, harm, or deter one or more pests that attach, invade, and/or are attracted to a plant, an animal, or product thereof.

In some embodiments, the pest control agent may optionally include a pesticide. In some embodiments, pesticides used in agricultural compositions may be selected from any of those listed in Table 7. Pesticides may include, but are not limited to parasiticides, insecticides, herbicides, antifungal or fungicide, anti-disease agents, behavior-modifying compounds, adhesives (e.g. gums), acaricide, algicide, avicide, bactericide, molluskicide, biocides, miticides, nematicide, rodenticide, and a virucide. Examples of pesticides include, but are not limited to, Bifonazole, Binapacryl, Bis(p-chlorophenoxy)methane, Bisphenol A, Bitertanol, Bromacil, Bromadiolone, Bromethalinlin, Bromophos, Bromopropylate, Bupirimate, Busulfan, Butrylin, Cambendazole, Candicidin, Candidin, Captan, Carbaryl, Carbendazim, Carbophenothion, Chloramben, Chloramphenacol, Chloranil, Chlorbetamide, Chlordimeform, Chlorfenac, Chlorphenesin, Chlorpyrifos, Chlorsulfuron, and Chlorothion. Any of the pesticides taught in United States Patent Publication US20030198659 may be useful in the present invention (the contents of which are herein incorporated by reference in their entirety).

The properties of SBPs allow advantages in pest control such as: a more tailored approach to the release rate of the agricultural compositions pest control agent, a lowered and more targeted environmental burden of the pest control agent, decreased numbers of required applications to the crop, stabilization of the pest control agent, the efficient coating of plant surfaces (e.g., leaves, bark, and/or roots), the efficient delivery of the pest control agent to the pest, the biodegradable nature of SBPs that are non-toxic to the environment. Depending on the need. SBPs can be developed that are tailored to the type of pest, local climate, geographical location, season, crop type, soil type, and other factors. The properties and advantages of SBPs will provide safe and effective options for agricultural protection that are more tailored to particular needs and which offer advantages over the current options.

In some embodiments the pest control agent may include a parasiticide. As used herein, the term, “parasiticide”, refers to any substance that harms, kills, retards, or otherwise inhibits the growth and/or reproduction of parasites. Parasiticides may be ectoparasiticides, i.e. parasiticides that are used to control ectoparasites that are located on the exterior of the corresponding host e.g. flies, ticks, mites, lice, fleas; or endoparasiticides i.e. parasiticides that are used to control parasites that are located inside the host e.g. roundworms, tapeworms and flukes; or endectocides i.e. control both external and internal parasites. In some embodiments, any of the insecticides described herein may be used as parasiticides. In some embodiments, any of the parasiticides described in Table 7 may be useful for the agricultural compositions described herein.

In some embodiments, the pest control agent may include an insecticide. As used herein, the term, “insecticide”, refers to any substance that harms, kills, retards, or otherwise inhibits the growth and/or reproduction of insects. Insecticides may include, but are not limited to, abamectin, allosamidin, doramectin, emamectin, eprinomectin, ivermectin, milbemectin, selamectin, spinosad, thuringiensin, calcium arsenate, copper acetoarsenite, copper arsenate, lead arsenate, potassium arsenite, or sodium arsenite; botanical insecticides such as anabasine, azadirachtin, d-limonene, nicotine, pyrethrins, cinerin I, cinerin II, jasmolin I, jasmolin II, pyrethrin I, pyrethrin II, quassia, rotenone, ryania, sabadilla, bendiocarb, carbaryl, benfuracarb, carbofuran, carbosulfan, decarbofuran, furathiocarb, dimetan, dimetilan, hyquincarb, pirimicarb, alanycarb, aldicarb, aldoxycarb, butocarboxim, butoxycarboxim, methomyl, nitrilacarb, oxamyl, tazimcarb, thiocarboxime, thiodicarb, thiofanox, allyxycarb, aminocarb, bufencarb, butacarb, carbanolate, cloethocarb, dicresyl, dioxacarb, ethiofencarb, fenethacarb, fenobucarb, isoprocarb, methiocarb, metolcarb, mexacarbate, promacyl, promecarb, propoxur, trimethacarb, xylylcarb, dinex, dinoprop, dinosam, barium hexafluorosilicate, cryolite, sodium fluoride, sodium hexafluorosilicate, sulfluramid, amitraz, chlordimeform, formetanate, formparanate, acrylonitrile, carbon disulfide, carbon tetrachloride, chloroform, chloropicrin, para-dichlorobenzene, 1,2-dichloropropane, ethyl formate, ethylene dibromide, ethylene dichloride, ethylene oxide, hydrogen cyanide, methyl bromide, methylchloroform, methylene chloride, naphthalene, phosphine, sulfuryl fluoride, tetrachloroethane, borax, calcium polysulfide, mercurous chloride, potassium thiocyanate, sodium thiocyanate, bistrifluron, buprofezin, chlorfluazuron, cyromazine, diflubenzuron, flucycloxuron, flufenoxuron, hexaflumuron, lufenuron, novaluron, noviflumuron, penfluron, teflubenzuron, triflumuron, epofenonane, fenoxycarb, hydroprene, kinoprene, methoprene, pyriproxyfen, triprene, juvenile hormone I, juvenile hormone II, juvenile hormone III, chromafenozide, halofenozide, methoxyfenozide, tebufenozide, α-ecdysone, ecdysterone, diofenolan, precocene I, precocene II, precocene III, dicyclanil, bensultap, cartap, thiocyclam, thiosultap, flonicamid, clothianidin, dinotefuran, thiamethoxam, nitenpyram, nithiazine, acetamiprid, imidacloprid, nitenpyram, thiacloprid, bromo-DDT, camphechlor, DDT, pp′-DDT, methoxychlor, pentachlorophenol, aldnm, chlorbicyclen, chlordane, chlordecone, dieldrin, dilor, endosulfan, endrin, heptachlor, isobenzan, isodrin, kelevan, mirex, bromfenvinfos, chlorfenvinphos, crotoxyphos, dichlorvos, dicrotophos, dimethylvinphos, fospirate, heptenophos, methocrotophos, mevinphos, monocrotophos, naled, naftalofos, phosphamidon, propaphos, schradan, tetrachlorvinphos, dioxabenzofos, fosmethilan, phenthoate, acethion, amiton, cadusafos, chlorethoxyfos, chlormephos, demephion, demephion-O, demephion-S, demeton, demeton-O, demeton-S, demeton-methyl, demeton-O-methyl, demeton-S-methyl, demeton-S-methylsulphon, disulfoton, ethion, ethoprophos, isothioate, malathion, methacrifos, oxydemeton-methyl, oxydeprofos, oxydisulfoton, phorate, sulfotep, terbufos, thiometon, amidithion, cyanthoate, dimethoate, ethoate-methyl, formothion, mecarbam, omethoate, prothoate, sophamide, vamidothion, chlorphoxim, phoxim, phoxim-methyl, azamethiphos, coumaphos, coumithoate, dioxathion, endothion, menazon, morphothion, phosalone, pyraclofos, pyridaphenthion, quinothion, dithicrofos, thicrofos, azinphos-ethyl, azinphos-methyl, dialifos, phosmet, isoxazole, isoxathion, zolaprofos, chlorprazophos, pyrazophos, chlorpyrifos, chlorpyrifos-methyl, butathiofos, diazinon, etrimfos, lirimfos, pirimiphos-ethyl, pirimiphos-methyl, primidophos, pyrimitate, tebupirimfos, quinalphos, quinalphos-methyl, athidathion, lythidathion, methidathion, prothidathion, isazofos, triazophos, azothoate, bromophos, bromophos-ethyl, carbophenothion, chlorthiophos, cyanophos, cythioate, dicapthon, dichlofenthion, etaphos, famphur, fenchlorphos, fenitrothion, fensulfothion, fenthion, fenthion-ethyl, heterophos, jodfenphos, mesulfenfos, parathion, parathion-methyl, phenkapton, phosnichlor, profenofos, prothiofos, sulprofos, temephos, trichlormetaphos-3, trifenofos, butonate, trichlorfon, mecarphon, fonofos, trichloronat, cyanofenphos, leptophos, crufomate, fenamiphos, fosthietan, mephosfolan, phosfolan pirimetaphos, acephate, isofenphos, methamidophos, propetamphos, dimefox, mazidox, mipafox, indoxacarb, acetoprole, ethiprole, fipronil, tebufenpyrad, tolfenpyrad, vaniliprole, acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, transfluthrin, etofenprox, flufenprox, halfenprox, protrifenbute, silafluofen, flufenerim, pyrimidifen, spiromesifen, chlorfenapyr, closantel, crotamiton, diafenthiuron, fenazaflor, fenoxacrim, flucofuron, hydramethylnon, isoprothiolane, malonoben, metoxadiazone, nifluridide, pyridaben, pyridalyl, rafoxanide, sulcofuron, triarathene and triazamate. In some embodiments, the insecticides may be any of those selected from Table 7, above.

In some embodiments, the pest control agent may include an herbicide. As used herein, the term “herbicide” refers to any substance that harms, kills, retards, or otherwise inhibits the growth and/or reproduction of unwanted plants. Herbicides may be specific to the unwanted plants or they may be generic, destroying all plants that come into contact with the herbicide. These herbicides may include, but are not limited to, chlorophenoxy acid herbicides, triazine herbicides, and organic phosphorus herbicides. Examples of herbicides include, but are not limited to, atrazine, cynazine, hexazinone, metribuzin, simazine, glyphosate, 2,4-D, 2,4,5-T, MCPA, and silvex. In some embodiments, the herbicides may be selected from any of those listed in Table 7, above.

In some embodiments, the pest control agent may include an antifungal agent. In some embodiments, anti-fungal agents described herein may also be referred to as fungicides. As used herein, the term “fungicide” refers to any substance that harms, kills, retards, or otherwise inhibits the growth and/or reproduction of fungi. Non-limiting examples of antifungal agents include: amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaftate, undecylenic acid, polyene antifungals imidazoles, triazoles, thiazoles, allylamines, echindocandans, aurones, balsam, orotomide, miltefosine, and crystal violet. Fungicides may also include, but are not limited to, phenol, pentachlorophenol, phenylmercuric oleate, copper 8-hydroxyquinoline, tributyltin chloride or triacetate, copper sulfate, and mercuric chloride. In some embodiments, any of the antifungal agents or fungicides provided in Table 7 may be used.

In some embodiments, the pest control agent may include behavior-modifying compounds. These compounds alter the behavior of the pests to limit the harm, irritation, discomfort, they may cause an organism. In some embodiments, the behavior modifying compound may be a mating disrupter, which reduces the overall population of the pest. Non-limiting examples of behavior modifying compounds include, but are not limited to pheromone, allomone, kairomone, capsaicin, a complex sugar, a phenolic compound, a monoterpenoid, dill, paprika, black pepper, catnip oil, chili powder, ginger, caffeine, red pepper, antifeedant, bird repellent, chemosterilant, insect attractant, insect repellent, mammal repellent, mating disrupter, and capsaicin oleoresin.

### Soil Stabilizers and Mechanics

In some embodiments, the SBP agricultural compositions may include soil or locus stabilizers. In some embodiments, SBPs may be soil stabilizers. Soil stabilization is the technique of changing the physical properties of a soil for a specific purpose. These properties may include, but are not limited to, the soil's weight bearing capabilities, tensile strength, and other aspects of soil performance known to those skilled in the art. In some embodiments, soil stabilizers may be selected chemicals, flowability agents, polymers, enzymes, surfactants, biopolymers, co-polymers, resins, ionic stabilizers, fiber reinforcements, salts, hydrophobic agents, and hydrophilic agents. In some embodiments, any of the soil stabilizers described in Table 7, above, may be used in SBPs.

### Biological Systems

In some embodiments, SBP agricultural compositions described herein include biological systems. These biological systems may include systems of symbiotes, microbiomes and/or probiotics. The compositions provided herein may include a SBPs and an active amount of beneficial microbes/probiotics. In some embodiments, SBPs may be used as stabilizers in the microbial compositions. In some embodiments, these microbiomes or symbiotes may incorporate species of fungi or bacteria. In some embodiments, the fungi are from the Aspergillus genus. In some embodiments, the bacteria are from the Streptomyces genus.

In some embodiments, the biological systems may be used to enable nitrogen fixation. These microbes, microorganisms, and/or microbiomes may incorporate rhizobia bacteria. Rhizobia bacteria enable nitrogen fixation in plants that do not independently fix nitrogen, such as legumes (Zahran et al. (1999) Microbiology and Molecular Biology Reviews 63(4):968-989, the contents of which are herein incorporated by reference in its entirety). In some embodiments, the biological systems described herein deliver rhizobia bacteria for the growth and production of other plants. In some embodiments, the SBP agricultural compositions described herein may be formulated with the nutrients needed to promote the growth of rhizobia bacteria. The beneficial microbe and/or probiotic can be any beneficial microbe and/or probiotic known in the art.

In some embodiments, SBP biological systems may include microbes, microorganisms, and/or microbiomes that promote plant growth. Such microbes, microorganisms, and/or microbiomes may include, but are not limited to, Algoriphagus ratkowskyi, Altererythrobacter luteolus, Alternaria thalcorgena, Arthrobacter agilis, Arthrobacter arilaitensis, Arthrobacter aurescens, Arthrobacter citreus, Arthrobacter crystallopoeietes, Arthrobacter globiformis, Arthrobacter humicola, Arthrobacter oryzae, Arthrobacter oxydans, Arthrobacter pascens, Arthrobacter ramosus, Arthrobacter tumbae, Aspergillus fumigatiaffinis, Bacillus aquimaris, Bacillus benzoevorans, Bacillus cibi, Bacillus herbersteinensis, Bacillus idriensis, Bacillus lichenformis, Bacillus niacin, Bacillus psychordurans, Bacillus simplex, Bacillus simplex I1, Bacillus simplex 237, Bacillus simplex 30N-5, Bacillus subtilis 30VD-1, Bartonella elizabethae, Citricoccus alkalitolerans, Citricoccus nitrophenolicus, Cladosporium sphaerospermum, Curtobacterium flaccumofciens, Exiguobacterium aurantiacum, Fusarium equiseti, Fusarium oxysporum, Georgenia ruani, Halomonas aquamarina, Kocuria rosea, Afassilia timonae, Mesorhizobium loti, Microbacterium aerolatum, Microbacterium oxydans, Aicrobacterium paludicola, Microbacterium paraoxydans, Microbacterium phyllosphaerae, Microbacterium testaceum, Micrococcus luteus, Mycobacterium sacrum, Nocardiopsis quinghaiensis, Oceanobacillus picturae, Ochroconis sp., Olivibacter soli, Paenibacilius tundrae, Penicillium chrysogenum, Penicillium commune, Phoma betae, Planococcus maritimus, Planococcus psychrotoleratus, Panomicrobium koreense, Planomicrobium okeanokoites, Promicromonospora kroppenstedtii, Pseudomonas brassicacearum, Pseudomonas fluorescens, Pseudomonas frederiksbergensis, Pseudomonas fulva, Pseudomonas geniculata, Pseudomonas gessardii, Pseudomonas libanensis, Pseudomonas mosselti, Pseudomonas plecoglossicida, Pseudomonas putda, Pseudomonas stutzeri, Pseudomonas syringae, Rhodococcus jostii, Sinorhizobium medicae, Sinorhizobium melioti, Staphylococcus succinus, Stenotrophomonas maltophilia, Stenotrophomonas rhizophila, Streptomyces althioticus, Streptomyces azureus, Streptomyces bottropensis, Streptomyces candiduts, Streptomyces chryseus, Streptomyces cirrahus, Streptomyces coenleofuscus, Streptomyces durmitorensis, Streptomyces flaveus, Streptomyces fradeiae, Streptomyces griseoruber, Streptomyces griseus, Streptomyces halstedii, Streptomyces marokkonensis, Streptomyces olivoviridis, Streptomyces peucetius, Streptomyces phaeochromogenes, Streptomyces pseudogriseolus, Terribacillus halophilus, Virgibacillus halodenitrificans, and/or Williamensia muralis. In further embodiments, such plant growth-promoting microbes, microorganisms, and/or microbiomes may be selected from any of those microbial isolates described in US Publication Number US20140342905, and International Publication Number WO2014201044, the contents of which are hereby incorporated by reference in their entirety.

In some embodiments, SBP biological systems may be used as biopesticides. As used herein, the term “biopesticide” refers to a composition with a bacteria, microorganism, or biological cargo that displays pesticidal activity. Any of the biopesticides taught in U.S. Pat. No. 6,417,163 and in Kumar et al. ((2017) Probiotics and Plant Health doi. 10.1007/978-981-10-3473-2_4) may be used herein (the contents of which are herein incorporated by reference in their entirety).

In some embodiments, SBP biological systems may be applied as a coating to a plant. The coating may be applied to the whole plant, or to any part of the plant described in the present disclosure. In some embodiments, the coating may be applied to a seed. In some embodiments, SBP biological systems may be used to prevent seed burning. In some embodiments, SBP biological systems may be environmentally friendly.

### Agricultural Therapeutic Agent

In some embodiments, agricultural applications involve the use of SBPs that are agricultural therapeutic agents or are combined with one or more agricultural therapeutic agents. As used herein, the term “therapeutic agent” refers to any substance used to restore or promote the health and/or well-being of a subject and/or to treat, prevent, alleviate, cure, or diagnose a disease, disorder, or condition. In some embodiments, the subject in the context of an agricultural therapeutic agent may refer to one or more plants. In some embodiments, the term subject in the context of an agricultural therapeutic agent may refer to one or more non-human animals. Examples of SBP therapeutic agents include, but are not limited to, adjuvants, analgesic agents, antiallergic agents, antiangiogenic agents, antiarrhythmic agents, antibacterial agents, antibiotics, antibodies, anticancer agents, anticoagulants, antidementia agents, antidepressants, antidiabetic agents, antigens, antihypertensive agents, anti-infective agents, anti-inflammatory agents, antioxidants, antipyretic agents, anti-rejection agents, antiseptic agents, antitumor agents, antiulcer agents, antiviral agents, biological agents, birth control medication, carbohydrates, cardiotonics, cells, chemotherapeutic agents, cholesterol lowering agents, cytokines, endostatins, enzymes, fats, fatty acids, genetically engineered proteins, glycoproteins, growth factors, health supplements, hematopoietics, herbal preparations, hormones, hypotensive diuretics, immunological agents, inorganic synthetic pharmaceutical drugs, ions, lipoproteins, metals, minerals, nanoparticles, naturally derived proteins, NSAIDs, nucleic acids, nucleotides, organic synthetic pharmaceutical drugs, oxidants, peptides, pills, polysaccharides, proteins, protein-small molecule conjugates or complexes, psychotropic agents, small molecules, sodium channel blockers, statins, steroids, stimulants, therapeutic agents for osteoporosis, therapeutic combinations, thrombopoietics, tranquilizers, vaccines, vasodilators. VEGF-related agents, veterinary agents, viruses, virus particles, and vitamins. Other therapeutic agents may include, but are not limited to, anthocyanidin, anthoxanthin, apigenin, dihydrokaempferol, eriodictyol, fisetin, flavan, flavan-3,4-diol, flavan-3-ol, flavan-4-ol, flavanone, flavanonol, flavonoid, furanoflavonols, galangin, hesperetin, homoeriodictyol, isoflavonoid, isorhamnetin, kaempferol, luteolin, myricetin, naringenin, neoflavonoid, pachypodol, proanthocyanidins, pyranoflavonols, quercetin, rhamnazin, tangeritin, taxifolin, theaflavin, thearubigin, chondrocyte-derived extracellular matrix, macrolide, erythromycin, roxithromycin, azithromycin and clarithromycin. In some embodiments, SBP therapeutics and methods of delivery may include any of those taught in International Patent Publication Numbers WO2017139684, WO2010123945, WO2017123383, or United States Publication Numbers US20170340575, US20170368236, and US20110171239 the contents of each of which are herein incorporated by reference in their entirety. In some embodiments, the agricultural therapeutic agent may be a pest control agent. In some embodiments, examples of pest control agents that may be useful as agricultural therapeutic agent include, but are not limited to parasiticides, insecticides, antifungal or fungicide, anti-disease agents, acaricide, algicide, avicide, bactericide, nematicide, and a virucide and are provided in Table 3 and Table 7.

In some embodiments, the agricultural therapeutic agent may be an antibiotic. As used herein the term antibiotic refers to any agent or substance that can kill, harm, or deter one or more microorganisms. Examples of antibiotics include, but are not limited to, amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipeneum, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, linomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, ciprofolaxin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, sulfadimethoxine, sulfamethizole, sulfamethoxazole, sulfanilimide, sulfasalazine, sulfisoxazole, demeclocycline, doxycycline, minocycline, oxytetracycline, tetracycline, clofazimine, dapsone, capreomycin, cycloserine, ethambutol, ethionamide, isoniazid, pyrazinamide, rifampicin, and streptomycin. In some embodiments, the antibiotics useful as therapeutic agents may include any of the antibiotics described in Table 7, above.

In some embodiments, the agricultural therapeutic agent may be nucleic acids. Nucleic acids may include DNA and/or RNA. In some embodiments, nucleic acids may be polynucleotides or oligonucleotides. Exemplary nucleic acids may include, but are not limited to, aptamers, plasmids, siRNA, microRNAs, or viral nucleic acids. In some embodiments, nucleic acids may encode a therapeutic peptide or protein, such as any one of those described herein. In some embodiments, SBPs may be used to improve the stability of composition comprising the nucleic acids. In some embodiments, SBPs may be used to facilitate the delivery of the nucleic acids to a plant.

### Agriculture Devices

In some embodiments, SBP agricultural compositions may be or may include may be used to improve the growth and production of agricultural products by utilizing said composition with an agricultural device. An agricultural device is a device or machine that assists in agricultural production. The SBP agricultural composition may comprise any format described in the present disclosure (e.g. hydrogel). In some embodiments, SBPs may be utilized as an agricultural device, as taught in in United States Patent Publication US20030198659 (the contents of which are herein incorporated by reference in its entirety). In some embodiments, SBPs may comprise one or more components of an agricultural device. In some embodiments, SBPs may be used in conjunction with another agricultural device. Agricultural devices that may incorporate SBPs include, but are not limited to, agricultural equipment, crop storage devices (e.g. bale bags), landscaping fabrics (e.g. polypropylene and burlap blankets), and pest control devices. In one embodiments, the agricultural equipment may comprise a silk-coated microporous pipeline, as taught in Chinese Patent Publication, CN102407193, the contents of which are herein incorporated by reference in their entirety.

In some embodiments, SBPs are or are used with agricultural devices used for pest control and are referred to as pest control agents. In some embodiments, SBPs that include one or more pest control agents are used as coatings to coat agricultural pest control devices. Devices may be carriers used to spread pest control agents included in carrier coatings. The carriers may be seeds. SBP seed coatings (e.g., seed coating compositions) provided herein may offer advantages with respect to the variety of cargo that can be formulated (small molecules, proteins, DNA, microbes, viruses), the ability to tailor the release rate of the cargo, stabilization of the cargo, efficient seed coating, break-down into non-toxic peptides, and/or a significantly reduced propensity to produce dust that can contaminate surrounding environments. The latter property, along with the controlled and delayed release of the active ingredient significantly reduces the contamination of surrounding environments by the active ingredient. These properties will likely mitigate the collateral damage to important pollinator populations. In addition, the compositions (e.g., seed coating compositions) provided herein impart advantages vs. seed flow and plantibility that are due to the physical properties of silk fibroin such as a very low coefficient of friction.

In some embodiments, SBP agricultural devices described herein may be used in the field of animal husbandry. In some embodiments. SBP agricultural devices described herein may be include a component or the whole of animal housing in the field of animal husbandry. SBPs may be used in animal housing applications to provide optimal temperature, humidity, radiation, air flow, precipitation and light required to keep the animal safe, healthy and comfortable.

Animals require healthy environments that permit the production, and quality of the non-human animals, as well as that of the animal agricultural products. Examples of animal housing include, but are not limited to, blankets, bedding, clothing, footwear (e.g. horseshoes), feeding equipment (e.g. bowls and water bottles), brushes, bandages, barns, coops, cages, stalls, liners, enclosures, ropes, ties, pens, flooring, shelters, sheds, stalls, ventilations systems, and wires.

In some embodiments, SBP agricultural devices may be used to aid the health and production of animals. In some embodiments, SBPs may be used in the treatment of mastitis. Transition from the dry period prior to lactation to lactation is a high-risk period for agricultural animals such as cows. During the period, the mammary gland (udder) may become infected with bacteria resulting in inflammation. In some embodiments, SBPs may be used in the treatment of mastitis. SBPs may be or may include antibiotics effective against one or more mastitis causing bacteria. SBPs may also be formatted into plugs and inserted into the teat canal (e.g., a teat sealant). In some embodiments, SBPs may be prepared as solutions and injected into the teat canal by an injection apparatus (e.g., a syringe, a needle, etc.). Formation of the plug may occur during injection and/or after injection. In some embodiments, SBPs may be formatted into films that is applied to the exterior of the teats. SBPs may be useful, both in treating and preventing mastitis.

### Aquaculture Products

In some embodiments, agricultural SBPs may be used as or in the preparation of aquaculture products. As used herein, the term “aquaculture” generally refers to the farming of aquatic animals (e.g., fish, crustaceans, mollusks) or the cultivation of aquatic plants (e.g., algae). As a non-limiting example, agricultural SBPs may be used in the preparation of aquaculture feeds for various aquatic animals including, but not limited to, carp, salmon, catfish, tilapia, cod, trout, milkfish, eel, shrimp, crawfish, crab, oyster, mussel, clam, jellyfish, sea cucumbers and sea urchins.

### Delivery

In some embodiments, the delivery of the SBP agricultural compositions described herein may occur through controlled release. In some embodiments, the SBP agricultural compositions may be utilized for the local delivery of cargo. In some embodiments, the agent may be a chemical for use in any one agricultural applications described in the present disclosure. In some embodiments, SBPs described herein may enable the controlled delivery of cargos that have a shorter half-life when delivered without SBPs, therein enhancing the time for which the therapeutic agent may be effective, as taught in United States Patent Publication US20100028451, the contents of which are herein incorporated by reference in its entirety. In some embodiments, SBPS may enhance the residence time of a cargo. In some embodiments the SBP delivery may be targeting to the entire plant, or animal; or it may be targeted to a portion of the plant or animal. In some embodiments, the portion of the plant may be leaf, root, bark, phloem, seed, and/or fruit.

In some embodiments, the controlled release of the SBPs for agricultural applications may be facilitated by diffusion of SBPs into the surrounding environment. This phenomenon has been observed in pharmaceutical compositions for animal subjects, as taught in United States Patent Publication No. US20170333351, the contents of which are herein incorporated by reference in its entirety. In some embodiments, the controlled release of SBPs for an agricultural application may be facilitated by the degradation and/or dissolution of SBPs. The degradation and/or dissolution has been employed for pharmaceutical compositions for animal subjects, as taught in International Patent Publications WO2013126799, WO2017165922, and U.S. Pat. No. 8,530,625, the contents of each of which are herein incorporated by reference in their entirety. In some embodiments, both the diffusion and the degradation and/or dissolution of SBPs may facilitate the controlled release of the agricultural compositions for agricultural applications.

In some embodiments, the delivery of the SBPs is controlled and/or maintained for one or more agricultural applications. In some embodiments, the agricultural compositions described herein maintain and/or improve the controlled delivery of the SBPs for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some embodiments, the SBPs described herein maintain and/or improve the controlled delivery of a payload for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, at least 1 month, at least 6 weeks, at least 2 months, at least 10 weeks, or at least 3 months.

In some embodiments, the SBPs may be released over a period of about 1 day to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, about 20 days, about 15 days, or about 10 days; about 10 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, about 20 days, or about 15 days; about 15 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, about 25 days, or about 20 days; about 20 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, about 30 days, or about 25 days; about 25 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, about 35 days, or about 30 days; about 30 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, about 40 days, or about 35 days; about 35 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, about 45 days, or about 40 days; about 40 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, about 50 days, or about 45 days; about 45 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, about 55 days, or about 50 days; about 50 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, about 60 days, or about 55 days; about 55 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, about 65 days, or about 60 days; about 60 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, about 70 days, or about 65 days; about 65 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, about 75 days, or about 70 days; about 70 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, about 80 days, or about 75 days; about 75 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, about 85 days, or about 80 days; about 80 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, about 90 days, or about 85 days; about 85 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, about 95 days, or about 90 days; about 90 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, about 100 days, or about 95 days; about 95 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, about 105 days, or about 100 days; about 100 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, about 110 days, or about 105 days; about 105 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, about 115 days, or about 110 days; about 110 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, about 120 days, or about 115 days; about 115 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, about 125 days, or about 120 days; about 120 days to about 200 days, about 195 days, about 190 days, about 185 days, about 180 days, about 175 days, about 170 days, about 165 days, about 160 days, about 155 days, about 150 days, about 145 days, about 140 days, about 135 days, about 130 days, or about 125 days; about 125 days to about 200 days, about 195 days, about 190 days, about 185 days, about 180 days, about 175 days, about 170 days, about 165 days, about 160 days, about 155 days, about 150 days, about 145 days, about 140 days, about 135 days, or about 130 days; about 130 days to about 200 days, about 195 days, about 190 days, about 185 days, about 180 days, about 175 days, about 170 days, about 165 days, about 160 days, about 155 days, about 150 days, about 145 days, about 140 days, or about 135 days; about 135 days to about 200 days, about 195 days, about 190 days, about 185 days, about 180 days, about 175 days, about 170 days, about 165 days, about 160 days, about 155 days, about 150 days, about 145 days, or about 140 days; about 140 days to about 200 days, about 195 days, about 190 days, about 185 days, about 180 days, about 175 days, about 170 days, about 165 days, about 160 days, about 155 days, about 150 days, or about 145 days; about 145 days to about 200 days, about 195 days, about 190 days, about 185 days, about 180 days, about 175 days, about 170 days, about 165 days, about 160 days, about 155 days, or about 150 days; about 150 days to about 200 days, about 195 days, about 190 days, about 185 days, about 180 days, about 175 days, about 170 days, about 165 days, about 160 days, or about 155 days; about 155 days to about 200 days, about 195 days, about 190 days, about 185 days, about 180 days, about 175 days, about 170 days, about 165 days, or about 160 days; about 160 days to about 200 days, about 195 days, about 190 days, about 185 days, about 180 days, about 175 days, about 170 days, or about 165 days; about 165 days to about 200 days, about 195 days, about 190 days, about 185 days, about 180 days, about 175 days, or about 170 days; about 170 days to about 200 days, about 195 days, about 190 days, about 185 days, about 180 days, or about 175 days; about 175 days to about 200 days, about 195 days, about 190 days, about 185 days, or about 180 days; about 180 days to about 200 days, about 195 days, about 190 days, or about 185 days; about 185 days to about 200 days, about 195 days, or about 190 days; about 190 days to about 200 days or about 195 days; or about 195 days to about 200 days.

The SBPs provided herein can be released e.g. at least 8% to about 100%, about 95%, about 90%, about 85%, about 80%, about 75%, about 70%, about 65%, about 60%, about 55%, about 50%, about 45%, about 40%, about 35%, about 30%, about 25%, or about 20%; about 20% to about 100%, about 95%, about 90%, about 85%, about 80%, about 75%, about 70%, about 65%, about 60%, about 55%, about 50%, about 45%, about 40%, about 35%, about 30%, or about 25%; about 25% to about 100%, about 95%, about 90%, about 85%, about 80%, about 75%, about 70%, about 65%, about 60%, about 55%, about 50%, about 45%, about 40%, about 35%, or about 30%; about 30% to about 100%, about 95%, about 90%, about 85%, about 80%, about 75%, about 70%, about 65%, about 600, about 55%, about 50%, about 45%, about 40%, or about 35%; about 35% to about 100%, about 95%, about 90%, about 85%, about 80%, about 75%, about 70%, about 65%, about 60%, about 55%, about 50%, about 45%, or about 40%; about 40% to about 100%, about 95%, about 90%, about 85%, about 80%, about 75%, about 700, about 65%, about 60%, about 55%, about 50%, or about 45%; about 45% to about 50% to about 100%, about 95%, about 90%, about 85%, about 80%, about 75%, about 70%, about 65%, about 60%, about 55%, or about 50%; about 50% to about 100%, about 95%, about 90%, about 85%, about 800, about 75%, about 700, about 65%, about 60%, or about 55%; about 55% to about 100%, about 95%, about 90%, about 85%, about 80%, about 75%, about 70%, about 65%, or about 60%; about 60% to about 100%, about 95%, about 90%, about 85%, about 80%, about 75%, about 70%, or about 65%; about 65% to about 100%, about 95%, about 90%, about 85%, about 80%, about 75%, or about 70%, about 70% to about 100%, about 95%, about 90%, about 85%, about 80%, or about 75%; about 75% to about 100%, about 95%, about 90%, about 85%, or about 80%; about 80% to about 100%, about 95%, about 90%, or about 85%; about 85% to about 100%, about 95%, or about 90%; about 90% to about 100%, or about 95%; or about 95% to about 100%, of the total amount of payload to be delivered.

### Applications

In some embodiments, the SBPs may be used in agricultural applications. SBPs may be used to increase biomass, increase product yield, and/or enhance offspring production of plants, plant agricultural products, animals, and animal agricultural products.

### Farming and Plant Characteristics

In some embodiments, SBPs may be used in the field of farming. As used herein, “farming” refers to the technique of growing crops, or keeping animals for food and materials. SBPs may be used in arable farming to grow crops, and/or pastoral farming SBPs may be utilized to improve one or more aspects of farming such as, but not limited to, plant growth, yield, reproduction, soil properties, weed control, pest control, disease control, product preservation, and/or treatment, environmental factors such as controlling access to water, air, and/or sunlight. In some embodiments, SBPs may be used to mitigate crop damage.

In some embodiments, SBPs may be used to promote plant growth. SBPs provided herein will allow increased tailoring of the agricultural composition according to plant type, seed type, soil characteristics, regional climate, local pathogens, pests, and application equipment. In some embodiments, SBPs applied to plants may result in enhanced growth of the plants or portions of plants. In some embodiments, the enhanced growth comprises a property selected from the group comprising improved plant vigor, increased plant weight, increased biomass, increased number of flowers per plant, higher grain and/or fruit yield, more tillers or side shoots, larger leaves, increased shoot growth, increased protein content, increased oil content, increased starch content, increased pigment content, increased chlorophyll content, and combinations thereof.

In some embodiments, the SBPs may be applied to the plant, or to a portion of the plant, as the plant, or portion of the plant, is growing. In some embodiments, the SBPs may be applied to the plant, or to a portion of the plant, after the plant, or portion of the plant, is harvested. In some embodiments, the locus of the plant is treated prior to the planting of seedlings or seeds. In some embodiments, plants are propagated from seeds and seedlings planted at the locus of treatment with SBPs described herein. In some embodiments, SBPs described herein are applied to one or more portions of plants. In some embodiments, the agricultural composition is applied to the plant, or to a portion of the plant, at the locus where the plant is growing. The locus may be the location in which the plant is growing. The locus may include but is not limited to a solid substrate e.g. soil, a liquid substrate e.g. water and a gaseous substrate e.g. air.

In some embodiments, SBPs provided herein will infer advantages to the growth and or development of the treated plants, including: optimal germination, protection of the roots, increasing the availability of nutrients, enhancing growth of the plant, increasing resistance of the plant to disease, deterring pathogens and pests, and increasing resistance of the plant to environmental conditions such as heat, flooding, and drought.

In some embodiments, the SBPs described herein increase the plants tolerance to stress factors selected from the group comprising a biotic stress factor and an abiotic stress factor. Non-limiting examples of a biotic stress factor include insects, arachnids, nematodes, weeds, and combinations thereof. Non-limiting examples of an abiotic stress include salt stress, water stress, ozone stress, heavy metal stress, cold stress, heat stress, nutritional stress, and combinations thereof.

In some embodiments, SBPs may be used to improve the reproduction of the plants. In some embodiments, SBPs may include pollinating material such as pollen that may applied to plants to facilitate fertilization. In some embodiments, SBPs may be used to improve plant health and resistance to diseases.

### Soil

In some embodiments, the SBPs of the present disclosure may be used to tune properties of soil. In some embodiments, the SBPs of the present disclosure are applied to the soil. In some embodiments, the SBPs described herein may be applied to soil prior to planting. In some embodiments, the SBPs described herein may be applied to soil in which a plant is already growing. In some embodiments, the SBPs of the present invention may be used to facilitate mulching, heat trapping, weed control, soil nutrition, soil pH, soil stability, and the mechanical properties of the soil. In some embodiments, the compositions provided herein can be contacted to a soil using crop dusting, painting, brushing, spraying, and/or injection.

In some embodiments, SBPs may include or may be applied to mulch, which may be may be used to facilitate the growth of a plant or agricultural product, as taught in Chinese Patent Publication, CN102733091 and CN102726257, (the contents of each of which are herein incorporated by reference in their entirety). Mulches may include natural mulches such as e.g. wood chips, bark, stone, pumice rock, gravels, organic, straw, paper, cardboard, grass clippings, compost, landscape fabric, saw dust, cocoa hull mulch, and pine straw, or decaying leaves; and artificial mulches such as plastic and paper. SBP mulches facilitate growth by controlling weed growth, shielding the soil from weather extremes, serving as a barrier for vapor and/or UV light, regulating temperature, and regulating moisture. In some embodiments, mulches may be contacted with SBPs of the present disclosure to facilitate growth of a plant or agricultural product and increase the yield of said plant or agricultural product. Mulches contacted with or SBP mulches described herein may be applied to the soil or locus in which the plant or agricultural product is being produced.

In some embodiments, the SBPs of the present invention may be used to facilitate growth of plants and agricultural products while reducing heat trapping. As used herein, the term “heat trapping” refers to the trapping of heat in the atmosphere, which may contribute to climate change. Heat trapping is, in part, caused by the release of chemicals (e.g. greenhouse gases) from the soil. The release of these chemicals from the soil is, in part, facilitated by the growth of bacteria in the soil as they ingest nutrients (e.g. nitrogen), as taught in Mellilo et al. (2017) Science 358(6359):101-115 (the contents of which are herein incorporated by reference in their entirety). Application of fertilizers has been demonstrated to increase the response of soil bacteria, and therefore increase the production of greenhouse gases, and the application of greater amounts of fertilizer further increases the production of greenhouse gases, as taught in Shcherbak et al. (2014) PNAS 111(25):9199-9204 (the contents of which are herein incorporated by reference in its entirety). In some embodiments, SBPs may be or may include fertilizers to provide controlled delivery. The controlled delivery of SBP fertilizer may reduce the amount of fertilizer needed to facilitate growth of plants and agricultural products, thereby enabling growth of said plants and agricultural products while reducing the amount of greenhouse gas produced.

In some embodiments, the SBPs of the present invention may be or may include photodegradable film. SBPs may be prepared to be photosensitive or SBPs may include photosensitive agents that degrade upon exposure to light, (see Chinese Patent Publication CN105199353 and International Patent Publication WO2017123383; the contents of each of which are herein incorporated by reference in their entirety). Photosensitive agents may be chemicals, small molecules, or a drug. Photodegradable SBPs may be prepared in any format (e.g. films, microspheres, nanospheres, and any format described in the present disclosure).

In some embodiments, SBPs of the present invention may be used to improve soil nutrition. The nutrition of soil can be tuned through delivery and/or controlled release of SBPs that may be or include nutrients, fertilizers, vitamins, and minerals. In some embodiments, the controlled release of such SBPs for soil nutrition may permit the use of lower dosages of nutrients, fertilizers, vitamins and minerals.

In some embodiments, the SBPs may be used to modulate soil pH. In some embodiments. SBPs may be or may include cargo that modulate soil pH including, but not limited to, chemicals, acids, bases, antibiotics, small molecules drugs, pesticides, herbicides, antibiotics, hydrophobic agents, hydrophilic agents, microbe, microorganism, and/or microbiome. Microbes, microorganisms, and/or microbiomes may modulate physical properties of their surrounding environment, as taught in Hartmann et al. (2014) The ISME Journal 8:226-244.

In some embodiments, the SBPs of the present invention may be used to modulate soil stability. As used herein, the term “soil stability” refers to the ability of soil or soil covered areas to move or withstand force. The stability of a soil is related to its mechanical properties, such as shear stress and strength. In some embodiments, SBPs may be or may include soil stability modulating agents such as flowability agents, polymers, enzymes, surfactants, biopolymers, co-polymers, resins, ionic stabilizers, fiber reinforcements, salts, hydrophobic agents, and hydrophilic agents. Methods of modulating soil stability involve covering said soil with a mat (see International Patent Publication No. WO20060706057; the contents of which are herein incorporated by reference in their entirety). In some embodiments, the SBPs of the present disclosure may be fabricated to a mat to control soil stability. These mats may be woven or non-woven. In some embodiments, the SBPs of the present invention may be used to alter the mechanical properties of the soil. Soil mechanical properties include, but are not limited to, shear strength, lateral earth pressure, consolidation, bearing capacity, permeability, seepage, and slope stability.

### Weed Control

In some embodiments, the SBPs of the present invention are used as agents of weed control. Non-limiting examples of weeds include Amaranth, Bermuda grass, Bindweed, Broadleaf plantain, Burdock. Common lambsquarters, Creeping Charlie, Dandelion, Goldenrod, Japanese knotweed, Kudzu, Leafy spurge, Milk thistle, Poison ivy, Ragweed, Sorrel, Striga, St. John's wort, Sumac, Tree of heaven, White clover. Wild carrot. Wood sorrel, and Yellow nutsedge. Some methods of controlling weed growth in soil involve covering said soil with a mat, as taught in International Patent Publication No. WO20060706057 (the contents of which are herein incorporated by reference in their entirety). In some embodiments, the SBPs may be utilized to fabricate a mat for weed control. These mats may be woven or non-woven. In some embodiments, the SBPs facilitate the delivery and/or controlled release of an herbicide.

### Seed Treatment and Storage

In some embodiments, seeds may be treated with SBPs to increase germination, seedling vigor, and seedling size. In some aspects, seeds may be treated with SBPs to increase seed storage, and shelf life of the seed, such that the seedlings produced upon germination of stored seeds are superior to seeds that stored without SBPs.

In some embodiments, the SBPs described herein may be used to enhance plant germination. As used herein, the term “germination” refers to growth from a seed or spore. In some embodiments, SBPs of the present disclosure may enhance plant germination by protecting seeds and spores from the surrounding environment. Non-limiting examples of such methods include SBP mulches or coverings. In some embodiments, the SBPs for enhanced germination are seed coatings. These include seed coatings with cargo such as micronutrients. In some embodiments, the SBPs of the present disclosure enhance plant germination by facilitating the delivery and/or controlled release of a cargo (e.g. nutrients, pesticides, herbicides, fertilizers). In some embodiments. SBPs may be or may include microbiomes to enhance germination.

SBPS may also be used to increase seedling vigor. As used herein, the term “seedling vigor” refers to the robustness of the seedling, as determined by its size, health, and growth rate. Seedling vigor may be tested by the cold test, the accelerated aging test, the electric conductivity test, the seedling vigor classification test, and any other method known to those skilled in the art. In some embodiments, the SBPs of the present disclosure increase seedling vigor by protecting said seedlings from the surrounding environment

In some embodiments, SBPs described herein may be used to increase seedling size. Seedling size can be measured by height, weight, biomass, growth rate, and any other method known to those skilled in the art. In some embodiments, the SBPs increase seedling size by protecting the seedlings from the surrounding environment e.g. mulches or coverings.

In some embodiments, the SBPs increase seedling vigor, and size by facilitating the delivery and/or controlled release of a cargo (e.g. nutrients, pesticides, herbicides, fertilizers).

### Animals

In some embodiments, SBPs may be used to improve characteristics of animal, and/or increase the yield and quality of animal agricultural products. In some embodiments, the agricultural products include, but are not limited to, milk, butter, cheese, yogurt, whey, curds, meat, oil, fat, blood, amino acids, hormones, enzymes, wax, feathers, fur, hide, bones, gelatin, horns, ivory, wool, venom, tallow, silk, sponges, manure, eggs, pearl culture, honey, and food dye.

In some embodiments, SBPs of the present disclosure may be used in animal agricultural products to facilitate the release of fragrance, flavor, or other compounds responsible for odor and/or flavor, as taught in United States Patent Publication No. US20150164117, the contents of which are herein incorporated by reference in their entirety.

In some embodiments, SBPs may incorporate animal feed or beverage. In some embodiments, SBPs may include health supplements, produce supplements, hormone supplements, and/or agricultural therapeutic agents to improve the health and viability of the animals. In some embodiments, SBPs may include animal feed such as forage, fodder, or a combination of forage and fodder. Examples of forage include, but are not limited to, plant derived material (e.g. leaves and stems), hay, grass, silage, herbaceous legumes, tree legumes, and crop residue. Examples of fodder include, but are not limited to, hay, straw, silage, compressed and pelleted feeds, oils, mixed rations, fish meal, meat and bone meal, molasses, oligosaccharides, seaweed, seeds, grains (e.g. maize, soybeans, wheat, oats, barley, rise, peanuts, corn, and sorghum), crop residues (e.g. stover, copra, straw, chaff and sugar beet waste), sprouted grains and legumes, brewer's spent grains, yeast extract, compounded feeds (e.g. meal type, pellets, nuts, cakes, and crumbles), cut grass and other forage plants, bran, concentrate mix, oilseed prescake (e.g. cottonseed, safflower, soybean peanut, and groundnut), horse gram, clipping waste, and legumes.

In some embodiments, SBPs described herein may be used to improve the yield of animal agricultural products by improving the health of non-human animals. In some embodiments, SBPs described herein may be used to improve the production capabilities of non-human animals. In some embodiments, SBPs described herein may be used to improve the breeding of non-human animals. In some embodiments, SBPs described herein may be used to improve the health, production, breeding, or a combination thereof in non-human animals.

In some embodiments, SBPs of this invention may be used to deliver health supplements to a non-human animal. These health supplements may improve the health of said non-human animals. SBPs may deliver said health supplements as a payload. SBPs may be incorporated into the feed, housing, or any other component or tool of animal husbandry that would enable the delivery of the payload. Examples of health supplements include, but are not limited to, vitamin A, vitamin B, vitamin C, vitamin D, vitamin E, vitamin K, thiamin, riboflavin, niacin, vitamin B6, vitamin B12, biotin, pantothenic acid, calcium, iron, phosphorus, iodine, magnesium, zinc, selenium, selenium, copper, manganese, chromium, molybdenum, chloride, potassium, nickel, silicon, vanadium, and tin.

In some embodiments, SBPs of this invention may be used to deliver supplements to a non-human animal that improve the yield and/or quality of the animal agricultural products. These health supplements may improve the production capabilities of said non-human animals. SBPs may include said supplements as a payload. Examples of supplements include, but are not limited to, vitamins, minerals, ions, nutrients, and hormones. In some embodiments, the SBPs may be used to stimulate animal appetite.

In some embodiments, SBPs of this invention may be used to deliver hormones to a non-human animal. SBPs may deliver said hormones as a payload. Examples of hormones include, but are not limited to, any steroid, dexamethasone, allopregnanolone, any estrogen (e.g. ethinyl estradiol, mestranol, estradiols and their esters, estriol, estriol succinate, polyestriol phosphate, estrone, estrone sulfate and conjugated estrogens), any progestogen (e.g. progesterone, norethisterone acetate, norgestrel, levonorgestrel, gestodene, chlormadinone acetate, drospirorenone, and 3-ketodesogestrel), any androgen (e.g. testosterone, androstenediol, androstenedione, dehydroepiandrosterone, and dihydrotestosterone), any mineralocorticoid, any glucocoriticoid, cholesterols, and any hormone known to those skilled in the art. In some embodiments, any of the hormones listed in Table 7 may be used with SBPs.

In some embodiments, SBPs of this invention may be used to deliver birth control agents to a non-human animal. These agents of disease control may improve the health, growth, and/or increase the yield of the agricultural product from said non-human animals. SBPs may be or may include birth control as cargo. SBPs may be incorporated into the feed, housing, or any other component or tool of animal husbandry that would enable the delivery of the payload. In some embodiments, SBPs may be used in conjunction with other forms of birth control, such as surgical procedures (e.g. spaying and neutering). Examples of birth control agents, include, but are not limited to, pills, ointments, implants, surgical procedures, hormones, patches, barriers, and injections.

In one embodiment, SBPs may be used to deliver birth control agents to cattle. Cattle birth control is important for producers to maintain herd genetic traits, reduce disease transmission, as well as eliminating the need for separate breeding pastures. The SBPs may provide controlled release of the birth control agent to the cattle. The birth control agents may include, but are not limited to, gonadorelin, gonadorelin acetate, progesterone, dinoprost tromethamine, and cloprostenol sodium, and any combination thereof.

### Pest Control

In some embodiments, SBPs may be used in pest control of plants, animals, plant agricultural products, and/or animal agricultural products. SBPs may be or may include pest control agents described herein. In some embodiments, SBP pest control devices may be used in pest control. Pest control agents and devices described herein may be applied directly to the pest; a pest susceptible surface such as the locus or planting substrate where the plant is growing e.g. soil; a pest habitat and/or the animal affected by the pest. In some embodiments, SBPs may be used to reduce the drift of a pest control agent to a surrounding environment.

### Disease Control

In some embodiments, SBPs may be useful in disease control of plants, and/or animals. In some embodiments, disease may be caused by disease agents. As used herein, the term “disease agent” refers to any biological pathogen that causes a disease. In some embodiments, the disease agent may be a parasite.

In some embodiments, the SBPs of the present disclosure may be used to treat plant diseases. In some embodiments, SBPs may promote disease resistance in plants. Disease control may include: (1) treating plants that are already infected, and (2) providing protection for plants that are yet to be infected. In some embodiments, SBPs may be administered to a plant or agricultural products that have the disease. In some embodiments, SBPs may be administered as a prophylactic treatment. In some embodiments, prophylactic treatment of non-infected plants or agricultural product may be achieved using SBPs that provide long-term protection against the disease, and/or are safe to the plant, the environment, and/or to public health. SBPs for disease control may be applied to the plant or agricultural product as a foliar spray. In some embodiments, the plants can be contacted with SBPs using crop dusting, painting, brushing, spraying, and/or injection.

In some embodiments, SBPs may be used in the disease control of diseases such as bacterial infections, aster yellows, bacterial wilt, blight (e.g. fire blight and rice bacterial blight), canker, crown gall, rot (e.g. bacterial rot, fungal rot, basal rot, gray mold rot, heart rot), basal rot, scab, fungal infections, anthracnose, black knot, citrus greening, fungal blight (e.g. chestnut blight and late blight), club root, damping-off. Dutch elm disease, ergot, Fusarium wilt, Panama disease, leaf blisters, mildew, downey mildew, powdery mildew, oak wilt, rust (e.g. blister rust, cedar apple rust, coffee rust), apple scab, smut, bunt, corn smut, snow mold, sooty mold, Verticillium wilt, viral infections, curly top, mosaic, psorosis, and spotted wilt.

In some embodiments, SBPs may be used to treat citrus greening. Citrus greening is a disease affecting citrus trees that is caused by an infection with the gram-negative bacterium, Candidatus liberibacter asiaticus (Las). The disease is also known as Huanglongbing (HLB) or yellow dragon disease. Citrus trees may include orange, grapefruit, lime, tangerine and/or lemon trees. No cure for citrus greening disease is known, and efforts to control it have been slow as the infecting pathogen resides in the difficult to access phloem of the infected tree. Affected trees have stunted growth, bear multiple off-season flowers (most of which fall off), and produce small, irregularly shaped fruit with a thick, pale peel that remains green at the bottom and tastes very bitter. In recent years the disease has spread to citrus orchards in the U.S., including Florida and California, and is putting the entire U.S. citrus crop at risk. Research has identified certain antibiotics with activity in killing or controlling the growth of Las (e.g., these antibiotics are exemplary Las inhibitory agents), these include: validoxylamine, actidione, ampicillin, carbenicillin, penicillin, cefalexin, rifampicin and sulfadimethoxine. In some embodiments, Las inhibitory agent may be a small molecule, a biologic, or a virus that has cytostatic and/or cytotoxic activity against Las. In some embodiments, SBPs may be formatted to coat a whole or a portion of a citrus tree, including, but not limited to, leaf, root, bark, and/or phloem.

In some embodiments, the present invention relates to the use SBPs as a matrix for formulations of disease inhibitory agents. In some embodiments, formulations of silk fibroin containing active ingredients with the ability to prevent the infection of plants, or of controlling disease in plants already infected with disease. More specifically, compositions including a silk fibroin and an inhibitory agent (e.g., 10 antibiotics with the ability to prevent the infection of citrus trees with Las, or of controlling citrus greening in citrus trees already infected with Las).

In some embodiments, the SBPs may be or may include therapeutic agents and/or agricultural therapeutic agents to enable disease control. SBPs offer advantages for treating plant disease in their ability to tune the release rate, stabilization, and are biodegradable. Depending on the need e.g. prophylactic vs. disease treatment, the SBPs can be developed to target different surfaces of the plant or agricultural product (e.g. leaves, bark, fruits, and roots). In addition, since high concentrations of therapeutic agents may be needed to reach the area of administration order to provide optimal disease treatment, local delivery (e.g. beneath the outer layer of bark and into the inner bark/phloem) of SBPs are possible. In some embodiments, hydrogels or other formats of SBPs described herein may be utilized to inject and form drug depots in the phloem, and provide effective and long-term treatment of affected plants or agricultural products, or protection of susceptible plants and agricultural products.

In some embodiments, SBPs of this invention may be used to deliver agents of disease control to a non-human animal. These agents of disease control may improve the health of said non-human animals. SBPs may deliver said agents of disease control as a payload. SBPs may be incorporated into the feed, housing, or any other component or tool of animal husbandry that would enable the delivery of the payload. In some embodiments, SBPs for disease control may be administered to treat a disease. In some embodiments, SBPs for disease control may be administered as a prophylactic to prevent the onset and/or spread of disease. Examples of agents of disease control include, but are not limited to, biologics, small molecules, vitamins, minerals, herbal preparations, health supplements, ions, metals, carbohydrates, fats, hormones, proteins, peptides, antibiotics and other anti-infective agents, hematopoietics, thrombopoietics, agents, antidementia agents, antiviral agents, antiangiogenic proteins (e.g. endostatin), antitumoral agents (chemotherapeutic agents), antipyretics, analgesics, anti-inflammatory agents, anti-infective, antiulcer agents, antiallergic agents, antidepressants, psychotropic agents, cardiotonics, antiarrhythmic agents, vasodilators, antihypertensive agents such as hypotensive diuretics, antidiabetic agents, anti-rejection agents, anticoagulants, cholesterol lowering agents, therapeutic agents for osteoporosis, bone morphogenic proteins, bone morphogenic-like proteins, enzymes, vaccines, immunological agents and adjuvants, naturally derived proteins, genetically engineered proteins, chemotherapeutic agents, cytokines, growth factors (e.g. epidermal growth factor, fibroblast growth factor, insulin like growth factor I and II, transforming growth factors, and vascular endothelial growth factors), nucleotides and nucleic acids, steroids carbohydrates and polysaccharides, glycoproteins, lipoproteins, viruses and virus particles, conjugates or complexes of small molecules and proteins, or mixtures thereof, and organic or inorganic synthetic pharmaceutical drugs.

In some embodiments, SBPs may be used in treatment of any of the animal diseases disclosed in Table 8 or diseases resulting from exposure to any of the disease agents listed in Table 8.

TABLE 8
Animal diseases, disorders, or conditions

| Disease, disorder, or condition | Category |
|---|---|
| autoimmune hemolytic anemia | animal autoimmune |
| bullous pemphgoid | animal autoimmune |
| chronic inflammatory demyelinated polyneuropathy | animal autoimmune |
| discoid lupus erythematosus | animal autoimmune |
| immune mediated thrombocytopenia | animal autoimmune |
| irritable bowel syndrome | animal autoimmune |
| panepidermal pustular pemphigus | animal autoimmune |
| paraneoplastic pemphigus | animal autoimmune |
| pemphigus | animal autoimmune |
| pemphigus complex | animal autoimmune |
| pemphigus erythematosus | animal autoimmune |
| pemphigus foliaceus | animal autoimmune |
| pemphigus vulgaris | animal autoimmune |
| systemic lupus erythematosis | animal autoimmune |
| anus cancer | animal cancer |
| bile duct cancer | animal cancer |
| bladder cancer | animal cancer |
| bone cancer | animal cancer |
| brain cancer | animal cancer |
| breast cancer | animal cancer |
| carcinoma | animal cancer |
| cervical cancer | animal cancer |
| chest cancer | animal cancer |
| colon/rectum cancer | animal cancer |
| connective tissue cancer | animal cancer |
| endometrial cancer | animal cancer |
| esophageal cancer | animal cancer |
| eye cancer | animal cancer |
| gallbladder cancer | animal cancer |
| head and neck cancer | animal cancer |
| kidney cancer | animal cancer |
| larynx cancer | animal cancer |
| leukemia | animal cancer |
| liver cancer | animal cancer |
| lung cancer | animal cancer |
| Lymphoma | animal cancer |
| mouth cancer | animal cancer |
| nose cancer | animal cancer |
| ovarian cancer | animal cancer |
| pancreatic cancer | animal cancer |
| penile cancer | animal cancer |
| prostate cancer | animal cancer |
| sarcoma | animal cancer |
| skin cancer | animal cancer |
| small intestine cancer | animal cancer |
| spinal marrow cancer | animal cancer |
| stomach cancer | animal cancer |
| tailbone cancer | animal cancer |
| testicular cancer | animal cancer |
| throat cancer | animal cancer |
| thyroid cancer | animal cancer |
| uterine cancer | animal cancer |
| Acute hepatopancreatic necrosis disease | animal disease |
| Aflatoxicosis | animal disease |
| African swine fever | animal disease |
| Akabane | animal disease |
| animal disease | animal disease |
| Avian influenza (bird flu) | animal disease |
| Blue-green algae (cyanobacteria) | animal disease |
| Bluetongue | animal disease |
| Botulism | animal disease |
| Botulism in poultry | animal disease |
| Bovine ephemeral fever | animal disease |
| Bovine tuberculosis | animal disease |
| Bovine virus diarrhea | animal disease |
| Brucellosis | animal disease |
| Buffalo fly | animal disease |
| Campylobacteriosis | animal disease |
| Caprine arthritis encephalitis (CAE) | animal disease |
| Cat-scratch disease | animal disease |
| cauda equina syndrome | animal disease |
| cervical spondylomyelopathy | animal disease |
| Classical Swine fever | animal disease |
| Clostridial diseases | animal disease |
| congenital disorder | animal disease |
| Copper deficiency | animal disease |
| Cryptococcosis | animal disease |
| degenerative joint disease | animal disease |
| degenerative spinal joint disease | animal disease |
| developmental disorder | animal disease |
| diabetes | animal disease |
| dietary disease | animal disease |
| elbow dysplasia | animal disease |
| Enzootic bovine leucosis (EBL) | animal disease |
| Epizootic ulcerative syndrome (red-spot disease) | animal disease |
| Equine infectious anaemia (EIA) | animal disease |
| Equine influenza | animal disease |
| Equine viral arteritis (EVA) | animal disease |
| Foot and mouth disease | animal disease |
| Fowl cholera | animal disease |
| gestational diabetes | animal disease |
| Giardiasis | animal disease |
| hemophilia | animal disease |
| hip dysplasia | animal disease |
| hormonal disease | animal disease |
| Hydatid disease (hydatid cysts) | animal disease |
| hyperparathyroidism | animal disease |
| Infectious laryngotracheitis | animal disease |
| inflammatory joint disease | animal disease |
| insulin deficient diabetes | animal disease |
| insulin resistance diabetes | animal disease |
| intervertebral disc disease | animal disease |
| Japanese encephalitis | animal disease |
| Johne's disease | animal disease |
| latent autoimmune diabetes | animal disease |
| Legg-Perthes disease | animal disease |
| Leptospirosis | animal disease |
| Listeriosis | animal disease |
| Lumpy jaw | animal disease |
| luxated patella | animal disease |
| lyme disease | animal disease |
| Marek's disease | animal disease |
| marree disease | animal disease |
| Melioidosis | animal disease |
| metabolic disorder | animal disease |
| narcolepsy | animal disease |
| Neospora caninum | animal disease |
| Newcastle disease | animal disease |
| Nosema | animal disease |
| obesity | animal disease |
| ornithosis | animal disease |
| osteochondritis dissecans | animal disease |
| Ovine brucellosis | animal disease |
| Pimelea poisoning | animal disease |
| Psittacosis | animal disease |
| Q fever | animal disease |
| Rabies | animal disease |
| Rinderpest | animal disease |
| Salmonellosis | animal disease |
| Skin fluke infestation | animal disease |
| Sparganosis | animal disease |
| Spotty liver | animal disease |
| St George disease | animal disease |
| Strangles | animal disease |
| Swine influenza | animal disease |
| Swine vesicular disease | animal disease |
| Tetanus | animal disease |
| Tick fever | animal disease |
| Transit tetany | animal disease |
| Transmissible spongiform encephalopathies | animal disease |
| Tuberculosis (TB) | animal disease |
| Vesicular exanthema | animal disease |
| Vesicular stomatitis | animal disease |
| vibriosis | animal disease |
| von Willebrand's disease | animal disease |
| Warts | animal disease |
| White nose syndrome | animal disease |
| White spot disease | animal disease |
| Wobbler's syndrome | animal disease |
| Wooden tongue and lumpy jaw | animal disease |
| Acanthocephala | disease agent |
| Acari Varroa destructor | disease agent |
| Anthrax | disease agent |
| Australian bat lyssavirus | disease agent |
| Avian paramyxovirus | disease agent |
| Babesia | disease agent |
| Balamuthia mandrillaris | disease agent |
| Bed bug | disease agent |
| Body louse | disease agent |
| bot flies | disease agent |
| brood parasite | disease agent |
| Brucella ovis | disease agent |
| Calydiscoides euzeti | disease agent |
| Calyptra | disease agent |
| Cancellaria cooperii | disease agent |
| Candiru | disease agent |
| Cestoda | disease agent |
| cestode | disease agent |
| Chinese liver fluke | disease agent |
| chordate | disease agent |
| Clonorchis sinensis | disease agent |
| Cookiecutter shark | disease agent |
| Crab louse | disease agent |
| Cryptosporidium | disease agent |
| Cuckoo | disease agent |
| Culicidae | disease agent |
| Cyclospora cayetanensis | disease agent |
| Cymothoa exigua | disease agent |
| Diphyllobothrium latum | disease agent |
| dog roundworm | disease agent |
| Dracunculus medinensis | disease agent |
| Echinococcosis | disease agent |
| Entamoeba coli, Acanthamoeba | disease agent |
| Entamoeba histolytica | disease agent |
| Enterobius vermicularis | disease agent |
| Equine herpesvirus | disease agent |
| False cleanerfish | disease agent |
| Filariasis | disease agent |
| fish tapeworm | disease agent |
| flatworm | disease agent |
| fleas | disease agent |
| Fowl pox | disease agent |
| Giardia | disease agent |
| Glochidium | disease agent |
| Guinea worm | disease agent |
| Head louse | disease agent |
| headlice | disease agent |
| Hendra virus | disease agent |
| Hippoboscoidea Tsetse fly | disease agent |
| hirudinea | disease agent |
| Hood mockingbird | disease agent |
| Hookworm | disease agent |
| horse flies | disease agent |
| human beef tapeworm | disease agent |
| Human botfly | disease agent |
| human pork tapeworm | disease agent |
| hydatid tapeworm | disease agent |
| Leishmani donovani | disease agent |
| Leishmani mexicana | disease agent |
| Leishmani tropica | disease agent |
| Leishmania | disease agent |
| Lethacotyle vera | disease agent |
| Lice | disease agent |
| Lipoptena | disease agent |
| Loa loa | disease agent |
| Male Deep sea anglers | disease agent |
| Melophagus ovinus | disease agent |
| monogenean | disease agent |
| mosquitoes | disease agent |
| nematode | disease agent |
| Nipah virus | disease agent |
| Oestridae | disease agent |
| Oxpeckers | disease agent |
| Pea crab | disease agent |
| Pestivirus | disease agent |
| Phlebotominae | disease agent |
| Phthiraptera | disease agent |
| pinworm | disease agent |
| Plasmodium | disease agent |
| Plasmodium falciparum | disease agent |
| Plasmodium malariae | disease agent |
| Plasmodium vivax | disease agent |
| Protocotyle euzetmaillardi | disease agent |
| Protozoan | disease agent |
| Pseudorhabdosynochus | disease agent |
| pubic lice | disease agent |
| Pyramidellidae | disease agent |
| Ringworm | disease agent |
| roundworms | disease agent |
| Sacculina | disease agent |
| sand flies | disease agent |
| scabies | disease agent |
| Screw-worm fly | disease agent |
| sheep ked | disease agent |
| Siphonaptera | disease agent |
| Snubnosed eel | disease agent |
| Strongyloides stercoralis | disease agent |
| Tabanidae | disease agent |
| Taenia saginata | disease agent |
| Taenia solium | disease agent |
| Tantulocarida | disease agent |
| Tapeworm | disease agent |
| Ticks (Ixodoidea) | disease agent |
| Toxocara canis | disease agent |
| Toxoplasma gondii | disease agent |
| trematode | disease agent |
| Triatominae | disease agent |
| Trichinella | disease agent |
| Vampire bat | disease agent |
| Vampire finch | disease agent |
| vampire moth | disease agent |
| Whipworm | disease agent |
| arthritis | joint condition |
| joint fracture | joint condition |
| ligament disease | joint condition |
| muscle disease | joint condition |
| osteoarthritis | joint condition |
| rheumatoid arthritis | joint condition |
| ruptured anterior cruciate ligament | joint condition |
| tendon disease | joint condition |
| acute pain | pain |
| back pain | pain |
| breakthrough pain | pain |
| chronic pain | pain |
| dental pain | pain |
| incident pain | pain |
| inflammatory pain | pain |
| joint pain | pain |
| knee pain | pain |
| musculoskeletal pain | pain |
| neuropathic pain | pain |
| nociceptive pain | pain |
| operative pain | pain |
| Pain | pain |
| post-operative pain | pain |
| psychogenic pain | pain |
| visceral pain | pain |
| Cattle ticks | parasitic agent |
| Acanthamoeba keratitis | parasitic disease |
| Ascariasis | parasitic disease |
| Babesiosis | parasitic disease |
| Cryptosporidiosis | parasitic disease |
| Cyclosporiasis | parasitic disease |
| dracunculiasis | parasitic disease |
| Enterobiasis | parasitic disease |
| fascioliasis | parasitic disease |
| gnathostomiasis | parasitic disease |
| Granulomatous amoebic encephalitis | parasitic disease |
| Leishmaniasis | parasitic disease |
| Malaria | parasitic disease |
| myiasis | parasitic disease |
| Onchocerciasis | parasitic disease |
| paragonimiasis | parasitic disease |
| Primary amoebic meningoencephalitis | parasitic disease |
| Schistosomiasis | parasitic disease |
| Strongyloidiasis | parasitic disease |
| swimmer's itch | parasitic disease |
| Toxocariasis | parasitic disease |
| Toxoplasmosis | parasitic disease |
| tungiasis | parasitic disease |

### Agricultural Product Preservation and Treatment

In some embodiments, agricultural products may be treated with SBPs to improve preservation, the shelf life, the physical appearance, and/or freshness of the agricultural products. In some aspects, agricultural products may be treated with SBPs to preserve the products such that they are superior in nutrition and appearance to products untreated agricultural products.

SBPs may be used to enhance the stability and shelf life of food and food products, as taught in Marelli et al. (2016) Scientific Reports 6:25263. In some embodiments, the SBPs of the present disclosure may be used as a coating to improve the stability and shelf life of agricultural products for human consumption. In some embodiments, the SBPs disclosed herein may improve the stability and shelf life of food and food products via the delivery and/or controlled release of a payload that may slow degradation. Non-limiting examples of payloads that may slow degradation includes preservatives and antibiotics.

In some embodiments, SBPs herein may be used to label agricultural products, as taught in International Patent Publication No. WO2009155397, the contents of which are herein incorporated by reference in their entirety. The resulting labels made from processed silks may be edible, biodegradable, and holographic. In some embodiments, SBPs of the present disclosure may be used in agricultural products to facilitate the release of fragrance, flavor, or other compounds responsible for odor and/or flavor, as taught in United States Patent Publication No. US20150164117, the contents of which are herein incorporated by reference in their entirety.

In some embodiments, SBPs may include agricultural products related to animals. SBPs may improve the stability and/or biodegradability of such products. In some embodiments, the agricultural products may be, meat, eggs, milk, hide, wool, honey, blood, plasma, animal feed, and fertilizer.

### Access to Water, Air and Light

In some embodiments, SBPs described herein may be used to control the access of the plant, animal or agricultural product to environmental factors such as water, air and/or sunlight. In some embodiments, SBPs may be used to modulate different aspects of the environment such as, but are not limited to, water, air, humidity, and light. In some embodiments, SBPs of the present disclosure may be used to increase the amount of water accessible to the plant, animal or agricultural product. In some embodiments, SBPs may be formatted into sachets for water transportation (see Chinese Patent Publication, CN102407193, the contents of which are herein incorporated by reference in its entirety. In some embodiments, the SBPs of the present invention may be used in pipelines. In some embodiments, the SBPs described herein may modulate the surrounding environment by controlling weed growth, shielding the soil from weather extremes, serving as a barrier for vapor and/or UV light, regulating temperature, and regulating moisture.

Formats of SBP for environmental control include, but are not limited to, sprays, solutions, hydrogels, rods, mats, powders, fabrics, emulsion, and any other format taught in the present disclosure. In some embodiments, SBPs may be used to prepare a material, such as a membrane, for air filtration. In some embodiments, SBPs may be used to prepare a material, such as a film, that modulates the light in use for an agricultural application.

In some embodiments, the SBPs may enable production of a plant, animal or agricultural product while reducing their climate change contributions. In some embodiments, SBPs of the present invention may be used for the controlled delivery of payloads known to contribute to the production of greenhouse gases.

### IV. Material Science Applications

In some embodiments, SBPs may be prepared for use in one or more material science applications. As used herein, the term “material science application” refers to any method related to development, production, synthesis, use, degradation, or disposal of materials. As used herein, the term “material” refers to a substance or chemical substance that may be used for the fabrication, production, and/or manufacture of an article. SBPs may be materials or may be combinations of processed silk with one or more materials. Examples of materials include, but are not limited to, adhesives, aquaculture products, biomaterials, composting agents, conductors, devices, electronics, emulsifiers, fabrics, fibers, fillers, films, filters, food products, heaters, insulators, lubricants, membranes, metal replacements, micelles, microneedles, microneedle arrays, microspheres, nanofibers, nanomaterials, nanoparticles, nanospheres, paper, paper additives, particles, plastics, plastic replacements, polymers, sensors, solar panels, spheres, sun screens, taste-masking agents, textiles, thickening agents, topical creams or ointments, optical devices, vasolines, and composites thereof. In some embodiments, materials comprising SBPs described herein may be used as a plastic, plastic supplement, or a plastic replacement, as taught in Yu et al, and Chantawong et al. (Yu et al. (2017) Biomed Mater Res A doi. 10.1002/jbm.a.36297; Chantawong et al. (2017) Mater Sci Mater Med 28(12):191), the contents of which are herein incorporated by reference in their entirety.

In some embodiments, the SBP is used as an excipient in materials.

### Consumer Products

In some embodiments, materials comprising SBPs may be used to produce or may be incorporated into consumer products. As used herein, the term “consumer products” refers to goods or merchandise purchasable by the public. Consumer products may include, but are not limited to, agricultural products, therapeutic products, veterinary products, and products for household use. Non-limiting examples of consumer products include cleaning supplies, sponges, brushes, cloths, protectors, sealant, adhesives, lubricants, protectants, labels, paint, clothing, insulators, devices, bandages, screens, electronics, batteries, surfactants, synthetic clothing, laundry pods or tablets, dishwasher pods or tablets, glitter, disposable cups, disposable plates, disposable silverware (e.g. forks, knives, spoons), wet wipes, tires, tennis balls, glitter, cigarette butts, tea bags, and paint.

### Surfactant Materials

In some embodiments, SBP materials may be used as a surfactant. In some embodiments, SBP materials may reduce the surface tension of liquids. In some embodiments, the SBP materials may be used to tune the surface tension of liquids. In some embodiments, the SBP may be a surfactant. In some embodiments, the surfactant may be prepared from SBPs. In some embodiments, silk is used in the preparation of surfactant using any of the methods described in Chinese patent publication CN105380891, the contents of which are herein incorporated by reference in their entirety. In some embodiments, SBP surfactants may be more environmentally friendly than existing surfactants. In some embodiments, SBPs have the surface tension of water. In some embodiments, SBPs have the surface tension of tears.

### Lubricant

In some embodiments, SBP materials may be used as lubricants, to reduce friction between two or more surfaces. In some embodiments, the SBP is a lubricant. In some embodiments, the SBP is an excipient in a lubricant. In some embodiments, the SBP is prepared from processed silk, oils, water, and other materials as described in Chinese Patent Publication Number CN101725049, the contents of which are herein incorporated by reference in their entirety. Lubricants can be prepared from SBPs in many formats, including, but not limited to, capsules, coatings, emulsions, fibers, films, foams, gels, grafts, hydrogels, membranes, microspheres, nanoparticles, nanospheres, organogels, particles, powders, rods, scaffolds, sheets, solids, solutions, sponges, sprays, suspensions, and vapors. In some embodiments, an SBP lubricant may comprise silk microspheres. In some embodiments, the microspheres may be prepared with a phospholipid coating as described in United States Patent Application Publication Number US20150150993A1, the contents of which are herein incorporated by reference in their entirety. In some embodiments, the lubricants may be used on a material surface, non-limiting examples of which include gears, machinery, vacuums, plastics, threads, wood, furniture, and other items. In some embodiments, the lubricants may be used on a biological surface, non-limiting examples of which include bones, joints, eyes, and mucosal membranes. In some embodiments, the coefficient of friction of an SBP is approximately that of naturally occurring, biological and/or protein lubricants (e.g. lubricin). In some embodiments, SBPs may be incorporated into a lubricant. Such methods may include any of those presented in International Publication No. WO2013163407, the contents of which are herein incorporated by reference in their entirety. In some embodiments, processed silk and/or SBPs may be used as an excipient to prepare a lubricant.

### Device Materials

In some embodiments, SBP materials may be used in the fabrication, production, and/or manufacture of a device, e.g., as taught in European Patent Number EP2904133, U.S. Pat. No. 9,802,374, and United States Patent Application Publication Number US20170312387, the contents of each of which are herein incorporated by reference in their entirety. In some embodiments, the device is a medical device (e.g. surgical devices, implants, dental devices, dental implants, diagnostic device, hospital equipment, etc.). In some embodiments, the device is an electronic device (e.g. diagnostic device, hospital equipment, implants, etc.).

The tem “medical device” refers to any device, product, equipment or material having surfaces that contact tissue, blood, or other bodily fluids of a subject in the course of their use or operation. Exemplary medical devices include, but are not limited to, absorbable and nonabsorbable sutures, access ports, amniocentesis needles, arterial catheters, arteriovenous shunts, artificial joints, artificial organs, artificial urinary sphincters, bandages, biliary stents, biopsy needles, blood collection tubes, blood filters, blood oxygenators, blood pumps, blood storage bags, bolts, brain and nerve stimulators, calipers, cannulas, cardiac defibrillators, cardioverter defibrillators, castings, catheter introducers, catheter sheaths s, catheters, chemical sensors, clips or fasteners, contraceptive devices, coronary stents, dialysis catheters, dialysis devices, dilators, drain tubes, drainage tubes, drug infusion catheters and guidewires, electrodes, endoscopes, endotracheal tubes, feminine hygiene products, fetal monitors, Foley catheters, forceps, gastroenteric tubes, genitourinary implants, guide wires, halo systems, heart valves, hearing aids, hydrocephalus shunts, implants, infusion needles, inserters, intermittent urinary catheters, intraurethral implants, introducers, introducer needles, irrigators, joint prostheses, knives, long-term central venous catheters, long-term tunneled central venous catheters, long-term urinary devices, monitors, nails, nasogastric tubes, needles, neurological stents, nozzles, nuts, obdurators, orthopedic implants, orthopedic devices, osteoports, pacemaker capsules, pacemaker leads, pacemakers, patches, penile prostheses, peripheral venous catheters, peripherally insertable central venous catheters, peritoneal catheters, peritoneal dialysis catheters, personal hygiene items, pins, plates, probes, prostheses, pulmonary artery Swan-Ganz catheters, pulse generators, retractors, rods, scaffolding, scalpels, screws, sensors, short-term central venous catheters, shunts, small joint replacements, specula, spinal stimulators, stents, stints, stylets, suture needles, suturing materials, syringes, temporary joint replacements, tissue bonding urinary devices, tracheostomy devices, transducers, trocars, tubes, tubing, urethral inserts, urinary catheters, urinary dilators, urinary sphincters, urological stents, valves, vascular catheters, vascular catheter ports, vascular grafts, vascular port catheters, vascular stents, wire guides, wires, wirings, wound drains, wound drain tubes, and wound dressings.

In some embodiments, the medical device may be an ocular device, such as, but not limited to, contact lens (hard or soft), intraocular lens, corneal onlay, ocular inserts, artificial cornea and membranes, eye bandages, and eyeglasses.

In some embodiments, the medical device may be a dental device, such as, but not limited to, dental flossers, dental flossing devices, dental threaders, dental stimulators, dental picks, dental massagers, proxy brushes, dental tapes, dental fillings, dental implants, orthodontic arch wire, and other orthodontic devices or prostodontic devices.

In some embodiments, the device may be any one of the following devices: audio players, bar code scanners, cameras, cell phones, cellular phones, car audio systems, communication devices, computer components, computers, credit cards, depth finders, digital cameras, digital versatile discs (DVDs), electronic books, electronic games and game systems, emergency locator transmitters (ELTs), emergency position-indicating radio beacons (EPIRBs), fish finders, global positioning system (GPS), home security systems, image play back devices, mediplayers, mobile computers, mobile phones, MP3 players, music players, notebook computers, pagers, palm pilots, personal computers, personal digital assistants (PDAs), personal locator beacons, portable books, portable electronic devices, portable game consoles, radar displays, radios, remote control device, satellite phones, smart cards, smartphones, speakers, tablets, telephones (e.g. cellular and standard), televisions, video cameras, video players, automobiles, boats, and aircraft.

In some embodiments, SBPs materials are used as, or incorporated into, the coating materials of a device. In some embodiments, the coating may be functional, decorative or both. Coatings may be applied to completely cover the surface. Coating may also be applied to partially cover the surface. Devices coated with SBPs may be more biocompatible and/or less-immunogenic.

### Antibiotic Materials

In some embodiments, SBPs may be used as materials due to their antibiotic properties. Such methods may include any of those described in European Patent Number EP3226835 and Mane et al. (2017) Scientific Reports 7:15531, the contents of each of which are herein incorporated by reference in their entirety. These antibiotic properties may be a general property of SBPs. In some embodiments, SBPs materials with antibiotic properties may include antibiotic cargo. In some embodiments, SBP materials may include antibiotic wound-healing materials (e.g., see Babu et al. (2017) J Colloid Interface Sci 513:62-72, the contents of which are herein incorporated by reference in their entirety).

### Synthetic Materials

In some embodiments, SBP materials are combined with synthetic materials. Such SBPs may be used to form scaffolds (e.g., see Lo et al. (2017) J Tissue Eng Regen Med doi.10.1002/term.2616, the contents of which are herein incorporated by reference in their entirety). In some embodiments, SBPs described herein are utilized to coat other materials. Such SBPs may include any of those described in Ai et al. (2017) International Journal of Nanomedicine 12:7737-7750, the contents of which are herein incorporated by reference in their entirety. In some embodiments, SBPs include plastics (e.g. thermoplastics, bioplastics, polyethylene, ultra-high-molecular-weight polyethylene, polypropylene, polystyrene, and polyvinyl chloride). In some embodiments, SBPs include plastic replacements. In some embodiments, SBPs include electronic materials or insulators.

In some embodiments. SBPs include polyolefins, polymers, and/or particles. In some embodiments. SBP materials may be prepared and used according to the methods of preparation and use described in European Patent Numbers EP3226835, EP3242967, and EP2904133, United States Publication Numbers US20170333351 and US20170340575, and Cheng et al. (2017) ACS Appl Mater Interfaces doi.10.1021/acsami.7b13460, the contents of each of which are herein incorporated by reference in their entireties.

In some embodiments, SBPs may be used as a plastic replacement in various products. Conventional plastic is made from petroleum products, primarily oil. It does not biodegrade and is harmful to the environment. SBPs are an attractive alternative to synthetic plastics due to their biocompatibility and biodegradability. As a non-limiting example, SBPs may be used as a plastic replacement in the production of water bottles and food containers. As another non-limiting example, SBPs may be used as a plastic replacement in the preparation of coating materials on a fabric or a cloth. Coatings used on apparels, such as a waterproof jacket or athletic shirt, are generally made of synthetic polymers and may release micro-plastic particles into water during a wash cycle. Using SBPs in replacement of synthetic polymers may help eliminate this problem.

### Nanomaterials

In some embodiments, SBPs include nanomaterials (e.g. nanoparticles, nanofibrils, nanostructures, and nanofibers), as taught in International Patent Application Publication No. WO2017192227, Xiong et al, and Babu et al. (Xiong et al. (2017) ACS Nano 11(12):12008-12019; Babu et al. (2017) J Colloid Interface Sci 513:62-72), the contents of each of which are herein incorporated by reference in their entirety. In some embodiments, the nanoparticles may include, but are not limited to, any of those listed in Table 1, above.

### Cosmetics

In some embodiments, SBPs are or used in the preparation of cosmetics. In some embodiments, SBPs are active substances in said cosmetics, e.g., as taught in U.S. Pat. No. 6,280,747 and United States Publication Number US20040170590, the contents of each of which are herein incorporated by reference in their entirety. In some embodiments, SBPs are added as a thickening agent, e.g., as taught in United States Publication Number US20150079012, the contents of which are herein incorporated by reference in their entirety. Examples of cosmetics include, but are not limited to, shampoos, conditioners, lotions, foundations, concealers, eye shadows, powders, lipsticks, lip glosses, ointments, mascara, gels, sprays, eye liners, liquids, solids, eyebrow mascaras, eyebrow gels, hairspray, moisturizers, dyes, minerals, perfumes, colognes, rouges, natural cosmetics, synthetic cosmetics, soaps, cleansers, deodorants, creams, towelettes, bath oils, bath salts, body butters, nail polish, hand sanitizer, primers, plumpers, balms, contour powders, bronzers, setting sprays, and setting powders.

In some embodiments, cosmetics may incorporate SBPs for stabilization and/or preservation of cosmetic components (e.g., see Li et al. (2017) Biomacromolecules 19(9):2900-2905, the contents of which are herein incorporated by reference in their entirety). In some embodiments, SBPs may be incorporated into cosmetics as a lubricant. Some SBPs may be used to facilitate release of fragrances, or other compounds responsible for odor (e.g., see United States Publication Number US20150164117, the contents of which are herein incorporated by reference in their entirety). In some embodiments, SBP cosmetics may be designed for topical applications (e.g., see U.S. Pat. No. 9,023,404, the contents of which are herein incorporated by reference in their entirety). Non-limiting examples of cosmetics that may be or may be combined with SBPs are listed in Table 9.

TABLE 9
Cosmetics
Cosmetics

adhesive
balm
bath oil
bath salt
body butter
bronzer
cleanser
cologne
concealer
conditioner
contour powder
cream
deodorant
dye
eye liner
eye shadow
eyebrow filler
eyebrow gel
eyebrow mascara
eyebrow pencil
faux lashes
foundation
glue
gel
hairspray
hand sanitizer
lip filler
lip gloss
lip liner
lip pencil
lipstick
liquid
lotion
lubricant
mascara
mineral
moisturizer
nail polish
natural cosmetic
ointment
perfume
plumper
powder
primer
rouge
setting powder
setting spray
shampoo
soap
solid
spray
synthetic cosmetic
towelette

In some embodiments, SBPs may be used as a plastic replacement in the preparation of cosmetics. As a non-limiting example, SBPs may be formatted as microbeads to be used in replacement of plastic microbeads in facial scrubs and toothpastes. As a further example, SBPs may be used to replace plastic emulsifiers and/or stabilizing agents used in any of those cosmetics listed in Table 9.

### Thickening Agents

In some embodiments, SBPs may be or may be combined with thickening agents. As used herein, the term “thickening agent” refers to a substance used to increase viscosity of another material, typically without altering any properties of the other material. In some embodiments, SBP thickening agents may be used in paints, inks, explosives, cosmetics, foods, or beverages.

In some embodiments, SBP thickening agents may be used in products for human consumption (e.g., as taught in United States Publication No. US20150079012, the contents of which are herein incorporated by reference in their entirety). SBP biocompatibility, biodegradability, and low toxicity make SBPs attractive tools for thickening materials designed for human consumption. In some embodiments, SBP thickening agents may be used to increase the viscosity of a food item. Examples of food items include, but are not limited to, puddings, soups, sauces, gravies, yogurts, oatmeals, chilis, gumbos, chocolates, and stews. In some embodiments, SBP thickening agents may be used to increase the viscosity of beverages. Examples of beverages include, but are not limited to, shakes, drinkable yogurts, milks, creams, sports drinks, protein shakes, diet supplement beverages, and coffee creamers.

In some embodiments, SBP thickening agents may be added to cosmetics (e.g., as taught in United States Publication Number US20150079012, the contents of which are herein incorporated by reference in their entirety. Such cosmetic products may include, but are not limited to, shampoos, conditioners, lotions, foundations, concealers, eye shadows, powders, lipsticks, lip glosses, ointments, mascara, gels, sprays, eye liners, liquids, solids, eyebrow mascaras, eyebrow gels, hairspray, moisturizers, dyes, minerals, perfumes, colognes, rouges, natural cosmetics, synthetic cosmetics, soaps, cleansers, deodorants, creams, towelettes, bath oils, bath salts, body butters, nail polish, hand sanitizer, primers, plumpers, balms, contour powders, bronzers, setting sprays, and setting powders.

### Military Applications

In some embodiments, SBPs may be used in military applications. For example, SBPs may be incorporated in military fabrics. Such fabrics may be used in items such as, but not limited to, panchos, tents, uniforms, vests, backpacks, personal protective equipment (PPE), linings, cords, ropes, and cables, webbings, straps and sheaths, helmet coverings, flags, bedsheets and mattress fabrics, ribbons, hats, gloves, masks, boots, suits and belts. As another example, SBPs may be used in the manufacture of a military device or gear. Non-limiting examples of military devices or gears include goggles, sunglasses, telescopes, binoculars, monoculars, flashlight, torches, watches, compasses, whistle, shields, knee caps, water bottles, flasks, and cameo face paint.

### Definitions

Absolute value: As used herein, the term “absolute value” describes the magnitude of a numerical number or measurement. The magnitude is listed as a non-negative number, but it can represent both positive and negative values.

Active pharmaceutical agent (API): As used herein, the term “active pharmaceutical agent,” or “API,” describes the component of a pharmaceutical composition that exhibits biological activity.

Cumulative release percentage: As used herein, the term “cumulative release percentage” describes the total percentage of a factor released from a source or depot over the course of a release period. This percentage may be determined from the total mass of released factor divided by initial mass of the factor in the source or depot. The “daily release percentage” describes the cumulative release percentage of factor per day. This value may be calculated from the best fit line slope of a plot of cumulative release percentage over time.

Effective concentration: As used herein, the term “effective concentration” refers to the concentration of a compound or factor required to elicit a particular response. The concentration needed to elicit half of a complete response is referred to as the “half maximal effective concentration” or “EC₅₀.” The concentration of compound needed to elicit 80% of a complete response is referred to as the “EC₈₀”. Where the compound or factor is inhibitory, the concentration needed to reduce or inhibit the response by half is referred to herein as the half maximal inhibitory concentration, or “IC₅₀.”

Initial burst: As used herein, the term “initial burst” refers to a rate of factor release from a source or depot over an initial release period (e.g., after administration or other placement, for example in solution during experimental analysis) that is higher than rates during one or more subsequent release periods.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. The present invention is further illustrated by the following nonlimiting examples.

### EXAMPLES

### Example 1. Formulation of Blank Silk Fibroin Rods

### Silk Fibroin Isolation

Silk yarn, purchased from Jiangsu SOHO International Group, was degummed to remove sericin. 30 grams of cut silk yarn were boiled at 100° C. in 3 L of deionized (DI) water with 0.02 M sodium carbonate for 240 minutes with stirring. The yarn was then transferred to a new boiling 0.02 M sodium carbonate aqueous solution and boiled at 100° C. for an additional 240 minutes with stirring. The total boiling time was discussed in terms of minute boil, or “mb.” The fibroin was then placed in DI water at 60-70° C. for 20 minutes with stirring, and then rinsed with clean DI water. This process was repeated 3 times. The fibroin was placed in clean DI water, stirred for 20 minutes, then rinsed with clean DI water, and this process was repeated for a total of 3×20 min.-rinse cycles. The fibroin was dried overnight, weighed, and dissolved at 20% (w/v) in a 9.3 M aqueous solution of lithium bromide (from Sigma-Aldrich, St. Louis, Mo.) for 5 hours at 60° C. The resulting fibroin solution was dialyzed against water at 4° C. in a 50 kDa regenerated cellulose dialysis tubing for 48 hours, with 6 water changes to remove the excess salt. The conductivity was recorded after each water change with a digital quality tester. When the conductivity was under 5 ppm, the fibroin solution was determined to be ready.

The resulting solution was centrifuged for 20 minutes at 3,900 RPM and 4° C. to remove insoluble particles. The supernatant was collected, and samples of the supernatant were diluted at 1:20 and 1:40 in water. Samples for a standard curve were prepared for an A280 assay by diluting pre-measured fibroin solutions to 5, 2.5, 1.25, 0.625, 0.3125, and 0 mg/mL in water. The silk concentration of the 1:20 and 1:40 diluted silk fibroin samples was measured against the standard curve by the absorbance at 280 nm.

The fibroin solutions were diluted to a final concentration of 3% (w/v) in 10 mM phosphate buffer (from Sigma Aldrich Fine Chemicals, St. Louis, Mo.), pH 7.4, and they were filtered through a 0.2 μm filter using a vacuum filter unit. 10 mL of each solution was aliquoted into 50 mL conical tubes, snap frozen in liquid nitrogen for 10 minutes, transferred for 20 minutes in −80° C., and lyophilized for 72 hours.

### Formulation of Silk Fibroin Rods

Lyophilized silk fibroin was dissolved in ultrapure water to obtain a concentration of 40% (w/v). The solution was extruded out of a syringe into tubing with a variety of diameters, dependent on the indication. For this example, the sample listed in Table 10 was extruded into approximately 12 cm lengths of 0.508 mm diameter polyetheretherketone (PEEK) (from Van Waters and Rogers (VWR), PA, USA, product 53500-690). The ends of the tubing were covered in parafilm, and the tubing was then incubated at 37° C. for 24 hours, after which it was cut to the necessary size, typically 2 cm lengths, frozen to −80° C. for at least four hours, and lyophilized. The final rods contained trace amounts of potassium phosphate buffer (with potassium phosphate dibasic and potassium phosphate monobasic). The final concentration of phosphate buffer was 133.3 mM.

TABLE 10
Samples of silk-fibroin rods

| | Silk Prep | Silk-Fibroin |
|---|---|---|
| Sample | Boil Time | Final % |
| No. | (min) | (w/w) |
| 1 | 480 | 100 |

The resulting rods were imaged via scanning electron microscopy (SEM). The rods were approximately 400 μm in diameter. The outer surfaces and cross-sectional surfaces of the silk-fibroin rods were smooth, with few to no ridges. The silk-fibroin rods were densely-packed, and the cross-sectional surfaces appeared smooth and contained few to no internal pores.

### Example 2. In Vitro Release of Small Molecules from 1 mm Silk Fibroin Rods

The silk yarn was purchased from Jiangsu SOHO International Group (Jiangsu, China). Lithium bromide was purchased from Sigma Aldrich (St. Louis, Mo.). The potassium phosphate monobasic and potassium phosphate dibasic were purchased from Sigma Aldrich Fine Chemicals (SAFC) (St. Louis, Mo.). The sodium carbonate and the sodium azide were purchased from Fisher Chemical (Waltham, Mass.). The celecoxib (CXB) was purchased from Cipla (Miami, Fla.).

### Silk Fibroin Isolation

Silk yarn, purchased from Jiangsu SOHO International Group, was degummed to remove sericin. 30 grams of cut silk yarn were boiled at 100° C. in 3 L of deionized (DI) water with 0.02 M sodium carbonate for 240 minutes with stirring. The yarn was then transferred to a boiling 0.02 M sodium carbonate aqueous solution and boiled at 100° C. for an additional 240 minutes with stirring. The fibroin was then placed in DI water at 60-70° C. for 20 minutes with stirring, and then rinsed with clean DI water. This process was repeated 3 times. The fibroin was placed in clean DI water, stirred for 20 minutes, then rinsed with clean DI water. This process was repeated for a total of three 20 minute rinse cycles. The fibroin was dried overnight, weighed, and dissolved at 20% (w/v) in a 9.3 M aqueous solution of lithium bromide (from Sigma Aldrich, St. Louis, Mo.) for 5 hours at 60° C. The resulting fibroin solution was dialyzed against water at 4° C. in a 50 kDa regenerated cellulose dialysis tubing for 48 hours, with 6 water changes to remove the excess salt. The conductivity was recorded after each water change with a digital quality tester. When the conductivity was under 5 ppm, the fibroin solution was determined to be ready.

The resulting solution was centrifuged for 20 minutes at 3,900 RPM and 4° C. to remove insoluble particles. The supernatant was collected, and samples of the supernatant were diluted at 1:20 and 1:40 in water. Samples for a standard curve were prepared for an A280 assay by diluting pre-measured fibroin solutions to 5, 2.5, 1.25, 0.625, 0.3125, and 0 mg/mL in water. The silk concentration of the 1:20 and 1:40 diluted silk fibroin samples was measured against the standard curve by the absorbance at 280 nm.

The silk fibroin solutions were diluted to a final concentration of 3% (w/v) in 10 mM phosphate buffer (from Sigma Aldrich Fine Chemicals, St. Louis Mo.), pH 7.4, and they were filtered through a 0.2 μm filter using a vacuum filter unit. 10 mL of each solution was aliquoted into 50 mL conical tubes, snap frozen in liquid nitrogen for 10 minutes, transferred for 20 minutes in −80° C., and lyophilized for 72 hours.

### 1 mm Silk Fibroin Rod Preparation

Lyophilized silk fibroin was dissolved with ultrapure water to obtain silk concentrations of 20, 30, and 40% (w/v). The relevant amount of celecoxib (CXB) (from Cipla, Miami Fla.) was weighed into a 4 mL glass vial. 250 μL of the relevant silk-fibroin solution (for example, Samples 8-58-1 through 8-58-3 use 250 μL of 20% (w/v) silk-fibroin to reach 50 mg) were then added to the dry CXB. The vial was briefly vortexed. A metal spatula was then used to manually mix the suspension until it became homogeneous. Using the spatula, the viscous suspension was loaded into the back of a 1 cc. syringe. The viscous mixture was then extruded out of the syringe into tubing with a variety of diameters, dependent on the indication. For this example, the samples were extruded into approximately 12 cm lengths of 1 mm diameter of either silicon (Grainger, Ill., USA, product number 2VLW4) or polytetrafluoroethylene (PTFE) tubing (from Van Waters and Rogers (VWR), PA, USA) The tubing was sealed with parafilm on both ends and left at 37° C. overnight to induce gelation. The tubing was then cut to the necessary size, typically 2 cm lengths. When the mixture was extruded from the tubing, the rods were found to hold their shape. The mixture was then frozen at −80° C. for at least four hours, either within or outside of the tubing. The resulting rods were then lyophilized for approximately 24 hours. Rods were removed from the tubing after lyophilization.

The rods are described in Table 11, alongside the concentration of silk solution used in their formulation, the total mass of silk fibroin used to formulate the rods, the total mass of CXB used to formulate the rods, and the theoretical loading percentages of the silk-fibroin and CXB in each sample. The term theoretical loading percentage refers to the assumed percentage of a component incorporated in a substance or product. The product may be an SBP. The component may be silk fibroin or CXB. The theoretical loading percentage may be in terms of either w/w percentage, w/v percentage, or v/v percentage. The samples were named by the process used to prepare and formulate each silk rod. For example, the sample named “480 mb; 1 mm; 20% st; 50mgsf; 150mgcxb; lyo; 25% sf; 75% cxb;” refers to a silk fibroin rod prepared from silk degummed with a 480-minute boil, an extrusion with a 1 mm diameter, a preparation from a 20% stock solution of silk fibroin, a preparation from 50 mg of silk fibroin, a preparation from 150 mg of celecoxib, lyophilization, a theoretical w/w percentage of 25% silk fibroin, and a theoretical w/w percentage of 75% celecoxib. The final rods contained trace amounts of potassium phosphate buffer (with potassium phosphate dibasic and potassium phosphate monobasic). The final concentration of phosphate buffer could be converted to (w/w) percentage by multiplying the concentration (in mM) by 0.0167.

TABLE 11
Theoretical silk fibroin and celecoxib percentages for 1 mm silk rod samples

| | | Formulation | Silk- | | Silk- | | Phosphate |
|---|---|---|---|---|---|---|---|
| | | Stock Silk | Fibroin | CXB | Fibroin | CXB | Buffer |
| Sample | | Concentration | Mass | Mass | Final % | Final % | Concentration |
| No. | Sample Name | (w/v %) | (mg) | (mg) | (w/w) | (w/w) | (mM) |
| 8-58-1 | 480 mb; 1 mm; 20% | 20 | 50 | 150 | 25 | 75 | 41.7 |
| | st; 50 mgsf; 150 | | | | | | |
| | mgcxb; lyo; 25% | | | | | | |
| | sf; 75% cxb | | | | | | |
| 8-58-2 | 480 mb; 1 mm; 20% | 20 | 50 | 200 | 20 | 80 | 37 |
| | st; 50 mgsf; 200 | | | | | | |
| | mgcxb; lyo; 20% | | | | | | |
| | sf; 80% cxb | | | | | | |
| — | 480 mb; 1 mm; 20% | 20 | 50 | 250 | 16.7 | 83.3 | 33.3 |
| | st; 50 mgsf; 250 | | | | | | |
| | mgcxb; lyo; 16.7% | | | | | | |
| | sf; 83.3% cxb | | | | | | |
| 8-58-4 | 480 mb; 1 mm; 30% | 30 | 75 | 150 | 33.3 | 66.7 | 62.5 |
| | st; 75 mgsf; 150 | | | | | | |
| | mgcxb; lyo; 33.3% | | | | | | |
| | sf; 66.7% cxb | | | | | | |
| 8-58-5 | 480 mb; 1 mm; 30% | 30 | 75 | 200 | 27.3 | 72.2 | 55.6 |
| | st; 75 mgsf; 200 | | | | | | |
| | mgcxb; lyo; 27.3% | | | | | | |
| | sf; 72.2% cxb | | | | | | |
| 8-58-6 | 480 mb; 1 mm; 30% | 30 | 75 | 250 | 23 | 77 | 50 |
| | st; 75 mgsf; 250 | | | | | | |
| | mgcxb; lyo; 23% | | | | | | |
| | sf; 77% cxb | | | | | | |
| 8-58-7 | 480 mb; 1 mm; 40% | 40 | 100 | 150 | 40 | 60 | 83.3 |
| | st; 100 mgsf; 150 | | | | | | |
| | mgcxb; lyo; 40% | | | | | | |
| | sf; 60% cxb | | | | | | |
| 8-58-8 | 480 mb; 1 mm; 40% | 40 | 100 | 200 | 33.3 | 66.7 | 74.1 |
| | st; 100 mgsf; 200 | | | | | | |
| | mgcxb; lyo; 33.3% | | | | | | |
| | sf; 66.7% cxb | | | | | | |
| 8-58-9 | 480 mb; 1 mm; 40% | 40 | 100 | 250 | 28.6 | 71.4 | 66.7 |
| | st; 100 mgsf; 250 | | | | | | |
| | mgcxb; lyo; 28.6% | | | | | | |
| | sf; 71.4% cxb | | | | | | |

The resulting silk fibroin rods were imaged via scanning electron microscopy (SEM), seen in FIG. 1A, FIG. 1B, FIG. 1C, and FIG. 1D. The rods were approximately 1000 μm in diameter. The silk-fibroin-CXB-rods were densely packed. The outer surfaces and cross-sectional surfaces of the silk-fibroin-CXB rods had ridges that appeared approximately 15 μm in length. The cross-sectional images of the silk-fibroin-CXB rods contained pores ranging in size from approximately 10-75 μm in length.

### In Vitro Release Experiments

The diameter of the silk-fibroin rods was measured using digital calipers. The rods were cut to 1 cm lengths to standardize release, and the weights of the rods were recorded. The density of the rods was calculated for each preparation. The rods from the tubing were placed into 45 mL of phosphate buffer (from Sigma Aldrich Fine Chemicals, St. Louis, Mo.), pH 7.4, 2% (v/v) Polysorbate-80 (from Croda, Snaith UK), and 0.05% (w/v) sodium azide (from Fisher Chemical, Waltham Mass.). This buffer ensured that the release was conducted under sink conditions (≥5× saturated solubility). The samples were incubated at 37° C. with gentle shaking. 1 mL of the release medium was taken at each timepoint (typically 1, 4, 7, 10, and 14 days and then weekly thereafter) and replaced with fresh media. The release medium was then analyzed via ultra-performance liquid chromatography (UPLC) to determine CXB concentration.

The silk fibroin rods demonstrated near zero-order kinetics for CXB release, with a low initial burst of 5-20%. The release rates of CXB were tuned by altering the density, CXB loading, and silk fibroin concentration. The CXB was released over the course of 1-3 months.

### Example 3. In Vitro Release of Small Molecules from 0.5 mm Silk Fibroin Rods

The silk yarn was purchased from Jiangsu SOHO International Group (Jiangsu, China). Lithium bromide was purchased from Sigma Aldrich (St. Louis, Mo.). The potassium phosphate monobasic and potassium phosphate dibasic were purchased from Sigma Aldrich Fine Chemicals (SAFC) (St. Louis, Mo.). The sodium carbonate and the sodium azide were purchased from Fisher Chemical (Waltham, Mass.). The celecoxib (CXB) was purchased from Cipla (Miami, Fla.).

### 0.5 mm Silk Fibroin Rod Preparation

Silk-fibroin (from Jiangsu SOHO International Corporation) was isolated as described in the preparation of the silk fibroin rods with no additives. Briefly, silk yarn, purchased from Jiangsu SOHO International Group, was degummed to remove sericin. 30 grams of cut silk yarn were boiled at 100° C. in 3 L of deionized (DI) water with 0.02 M sodium carbonate with stirring. The yarn was then transferred to a new boiling 0.02 M sodium carbonate aqueous solution and boiled at 100° C. for additional time with stirring. The total boiling time was discussed in terms of minute boil, or “mb.” The silk fibroin was boiled for either a total time of 480 or 120 minutes while being degummed. The total boiling time was discussed in terms of minute boil, or “mb.” Longer boiling times produced silk fibroin with lower average molecular weights of approximately 5-60 kDa.

The fibroin was then placed in DI water at 60-70° C. for 20 minutes with stirring, and then rinsed with clean DI water. This process was repeated 3 times. The fibroin was placed in clean DI water, stirred for 20 minutes, then rinsed with clean DI water, and this process was repeated for a total of 3×20 min.-rinse cycles. The fibroin was dried overnight, weighed, and dissolved at 20% (w/v) in a 9.3 M aqueous solution of lithium bromide (from Sigma-Aldrich, St. Louis, Mo.) for 5 hours at 60° C. The resulting fibroin solution was dialyzed against water at 4° C. in a 50 kDa regenerated cellulose dialysis tubing for 48 hours, with 6 water changes to remove the excess salt. The conductivity was recorded after each water change with a digital quality tester. When the conductivity was under 5 ppm, the fibroin solution was determined to be ready. The silk fibroin solution was centrifuged for 20 minutes at 3,900 RPM and 4° C. to remove insoluble particles. Solutions were diluted to a final concentration of 3% (w/v) in 10 mM phosphate buffer, pH 7.4, filtered through a 0.22 μm filter, frozen in liquid nitrogen, and lyophilized for 72 hours.

Lyophilized silk-fibroin was dissolved with ultrapure water to obtain concentrations of 20, 30, and 40% (w/v). The relevant amount of CXB (from Cipla, Miami Fla.) was weighed into a 4-mL glass vial. 250 μL of the relevant silk-fibroin solution was then added to the dry CXB, and the vial was then briefly vortexed. A metal spatula was used to manually mix the suspension until it was homogeneous. Using the spatula, the viscous suspension was loaded into the back of a 1 cc. syringe. The viscous mixture was extruded out of the syringe into tubing with a variety of diameters, dependent on the indication. For this example, the samples listed in Table 12 were extruded into approximately 12 cm lengths of 0.508 mm diameter PEEK tubing (from Van Waters and Rogers (VWR), PA, USA, product 53500-690). The tubing was then sealed on both ends with parafilm and left at 37° C. for 24 hours or overnight for gelation. The tubing was cut to the necessary size, typically 2 cm lengths. Half of the samples were frozen to −80° C. for at least four hours and lyophilized, while half of the samples were oven dried at 60° C. for 16 hrs. The samples were named by the process used to prepare and formulate each silk rod. For example, the sample named “480 mb; 0.5 mm; 40% st; 100mgsf; 200mgcxb; lyo; 33.3% sf; 66.7% cxb” refers to a silk fibroin rod prepared from silk degummed with a 480-minute boil, an extrusion with a 0.5 mm diameter, a preparation from a 40% stock solution of silk fibroin, a preparation from 100 mg of silk fibroin, a preparation from 200 mg of celecoxib, lyophilization, a theoretical w/w percentage of 33.3% silk fibroin, and a theoretical w/w percentage of 66.7% celecoxib. The final rods contained trace amounts of potassium phosphate buffer (with potassium phosphate dibasic and potassium phosphate monobasic). The final concentration of phosphate buffer could be converted to (w/w) percentage by multiplying the concentration (in mM) by 0.0167.

TABLE 12
Theoretical silk fibroin and celecoxib percentages for 0.5 mm silk rod samples

| | | Stock | Silk | | | | | |
|---|---|---|---|---|---|---|---|---|
| | | Concentration | Prep | Silk- | | Silk- | | Phosphate |
| | | of Silk for | Boil | Fibroin | CXB | Fibroin | CXB | Buffer |
| Sample | | Formulation | Time | Mass | Mass | Final % | Final % | Conc. |
| No. | Sample Name | (w/v %) | (min) | (mg) | (mg) | (w/w) | (w/w) | (mM) |
| | — | — | 480 | 50 | 200 | 20 | 80 | 37 |
| | — | — | 480 | 75 | 200 | 27.3 | 72.2 | 55.6 |
| | — | — | 480 | 100 | 100 | 50 | 50 | 95.2 |
| | — | — | 480 | 100 | 150 | 40 | 60 | 83.3 |
| 8-65-6 | 480 mb; 0.5 mm; | 40 | 480 | 100 | 200 | 33.3 | 66.7 | 74.1 |
| | 40% st; 100 mgsf; | | | | | | | |
| | 200 mgcxb; lyo; | | | | | | | |
| | 33.3% sf; 66.7% cxb | | | | | | | |
| | — | — | 480 | 100 | 250 | 28.6 | 71.4 | 66.7 |
| | — | — | 120 | 50 | 200 | 20 | 80 | 37 |
| | — | — | 120 | 75 | 200 | 27.3 | 72.2 | 55.6 |
| | — | — | 120 | 100 | 150 | 40 | 60 | 83.3 |

The resulting lyophilized rods were photographed (see FIG. 2A) of imaged via SEM (see FIG. 2B, FIG. 2C, and FIG. 2D). The rods were approximately 400 μm in diameter, and the rod in FIG. 2A, FIG. 2B, FIG. 2C, and FIG. 2D had a diameter of 430 μm. The silk-fibroin rods were densely packed with an even distribution of the API. The outer surfaces and cross-sectional surfaces of the silk-fibroin rods loaded with CXB had ridges that appeared approximately 15 μm in length. Furthermore, the cross-sectional images of the silk-fibroin rods with celecoxib contained few small pores.

### In Vitro Release Experiments

The rods were cut to 1 cm lengths to standardize release, and the weights of the rods were recorded. The densities of the rods were calculated for each preparation. The rods were placed into 45 mL of phosphate buffer, pH 7.4, 0.3% (v/v) Polysorbate-80 (from Croda, Snaith UK), and 0.05% (w/v) sodium azide (from Fisher Chemical, Waltham Mass.). This buffer ensured that the release was conducted under sink conditions (≥5× saturated solubility). A suspension of CXB containing 800 μg CXB was used as a control. The samples were incubated at 37° C. with gentle shaking. 1 mL of the release medium was taken at each timepoint (typically 1, 4, 7, 10, and 14 days and then weekly thereafter) and replaced with fresh media. The release medium was then analyzed via UPLC at 260 nm to determine CXB concentration.

The silk fibroin rods demonstrated near zero-order kinetics for CXB release, with a low initial burst of 15%. The release rates of CXB could be modulated by altering the silk molecular weight, CXB loading, and the method of drying the silk fibroin rods. The CXB was released over the course of 1-3 months. The rods with the 0.5 mm diameter displayed a faster release, when compared to the 1 mm rods, due to the larger surface area to volume ratio of the smaller rods.

### Example 4. In Vitro Release of Small Molecules from Silk Fibroin Gels

All formulations were prepared with silk yarn purchased from SOHO. The silk hydrogels were prepared with celecoxib (CXB) (from Cipla, Miami Fla.). The poloxamer-188 (P188), sodium chloride, and hydrochloric acid were from Sigma-Aldrich (St. Louis, Mo.), while the PEG4 kDa was from Clariant. Charlotte N.C. Polysorbate-80 was purchased from Croda (Snaith UK). Potassium phosphate monobasic and potassium phosphate dibasic were purchased from Sigma Aldrich Fine Chemical (SAFC, St. Louis Mo.). Phosphate buffered saline was purchased from Gibco (USA).

### Formulation of Silk Fibroin Hydrogels

Silk fibroin hydrogels were formulated with poloxamer-188 (P188) (from Sigma, St. Louis, Mo.) or polyethylene glycol 4000 Da (PEG 4k) (from Clariant, Charlotte N.C.). These hydrogels were formulated with celecoxib, the delivery of which was monitored. To prepare the formulations, a 27.8% suspension of celecoxib (CXB) in 0.79% polysorbate 80 as well as a stock solution of phosphate buffer (315 mM, pH=7.4) was used to dissolve either 120 mb or 480 mb silk fibroin and added to a syringe. Excipient solutions were then prepared with varying combinations of sodium chloride, PEG4 kDa, P188, and/or hydrochloric acid and added to a second syringe. Excipient solutions were prepared so that a 0.75:1 mix of silk-fibroin solution:excipient solution would result in the desired final formulations, with an osmolarity of 280 mOsm. The two syringes were then connected via a B Braun fluid dispensing connector, and the contents of the two syringes were mixed back and forth until homogeneous (at least 25 times). The syringes were then capped with a sterile syringe cap and incubated on a rotator at 37° C. for 24 hours. Syringes were stored at 4° C. until analysis.

Formulations were prepared as described in Table 13A and Table 13B, with either higher molecular weight (HMW or 120 mb, with an average molecular weight of 100-300 kDa) or low molecular weight (LMW or 480 mb, with an average molecular weight of about 30-60 kDa) silk fibroin. Longer boiling times, measured in “minute boil” or “mb”, produced silk fibroin with smaller molecular weights. The samples in Table 13A and Table 13B are named by the process used to prepare and formulate each hydrogel. For example, in the sample named 120 mb; hyd; 27.8% cxbst; 5% SFf; 10% CXBf; 40% PEG4kf, “120 mb” refers to silk degummed with a 120-minute boil, “hyd” refers to the formulation of the sample as a hydrogel, “27.8% cxbst” refers to a preparation from a stock solution of 27.8% of celecoxib, “5% SFf” refers to a formulation with 5% (w/v) silk fibroin, “10% CXBf” refers to a formulation with 10% (w/v) celecoxib, and “40% PEG4kf” refers to a formulation with 40% PEG 4 kDa. Some hydrogels were prepared with P188 (% P188f). The hydrogels were injectable through a 27-gauge, h inch needle. The hydrogels were formulated with varying silk fibroin molecular weights, gelling excipients, and silk fibroin concentrations. The hydrogels were formulated under aqueous conditions, with tight control of osmolarity and pH. The pH was measured with a B30PCI Benchtop Multi Parameter Meter—pH, Conductivity, ISE (VWR Catalog #89231-696), with a glass probe (VWR Catalog #89231-592). All hydrogels had a final phosphate buffer concentration of 22 mM.

TABLE 13A
Descriptions of hydrogels prepared loaded with celecoxib

| | | | Min. | Silk- | | | | NaCl | HCl |
|---|---|---|---|---|---|---|---|---|---|
| Sample | Sample | | Boil | fibroin | | Excipient | CXB | Conc. | Conc. |
| No. | name | Description | (mb) | Conc. % | Excipient | conc. % | Conc. % | (mg/mL) | (mM) |
| 168-1 | 120 mb; hyd; | 5% 120 mb | 120 | 5 | PEG 4k | 40 | 10 | 2.95 | 15 |
| | 27.8% cxbst; | with PEG 4k | | | | | | | |
| | 5% SFf; 10% | | | | | | | | |
| | CXBf; 40% | | | | | | | | |
| | PEG4kf | | | | | | | | |
| 168-2 | 120 mb; hyd; | 3% 120 mb | 120 | 3 | PEG 4k | 40 | 10 | 2.95 | 15 |
| | 27.8% cxbst; | with PEG 4k | | | | | | | |
| | 3% SFf; 10% | | | | | | | | |
| | CXBf; 40% | | | | | | | | |
| | PEG4kf | | | | | | | | |
| 168-3 | 120 mb; hyd; | 5% 120 mb | 120 | 5 | P188 | 10 | 10 | 5.97 | 0 |
| | 27.8% cxbst; | with P188 | | | | | | | |
| | 5% SFf; 10% | | | | | | | | |
| | CXBf; 10% | | | | | | | | |
| | P188f | | | | | | | | |
| 168-4 | 120 mb; hyd; | 3% 120 mb | 120 | 3 | P188 | 10 | 10 | 5.99 | 0 |
| | 27.8% cxbst; | with P188 | | | | | | | |
| | 3% SFf; 10% | | | | | | | | |
| | CXBf; 10% | | | | | | | | |
| | P188f | | | | | | | | |
| 168-5 | 480 mb; hyd; | 5% 480 mb | 480 | 5 | PEG 4k | 40 | 10 | 2.87 | 15 |
| | 27.8% cxbst; | with PEG 4k | | | | | | | |
| | 5% SFf; 10% | | | | | | | | |
| | CXBf; 40% | | | | | | | | |
| | PEG4kf | | | | | | | | |
| 168-6 | 480 mb; hyd; | 3% 480 mb | 480 | 3 | PEG 4k | 40 | 10 | 2.91 | 15 |
| | 27.8% cxbst; | with PEG 4k | | | | | | | |
| | 3% SFf; 10% | | | | | | | | |
| | CXBf; 40% | | | | | | | | |
| | PEG4kf | | | | | | | | |
| 168-7 | 480 mb; hyd; | 5% 480 mb | 480 | 5 | P188 | 10 | 10 | 5.90 | 0 |
| | 27.8% cxbst; | with P188 | | | | | | | |
| | 5% SFf; 10% | | | | | | | | |
| | CXBf; 10% | | | | | | | | |
| | P188f | | | | | | | | |
| 168-8 | 480 mb; hyd; | 3% 480 mb | 480 | 3 | P188 | 10 | 10 | 5.94 | 0 |
| | 27.8% cxbst; | with P188 | | | | | | | |
| | 3% SFf; 10% | | | | | | | | |
| | CXBf; 10% | | | | | | | | |
| | P188f | | | | | | | | |
| 168-9 | 480 mb; hyd; | 2% 480 mb | 480 | 2 | P188 | 10 | 10 | 5.96 | 0 |
| | 27.8% cxbst; | with P188 | | | | | | | |
| | 2% SFf; 10% | | | | | | | | |
| | CXBf; 10% | | | | | | | | |
| | P188f | | | | | | | | |

TABLE 13B
Properties of the hydrogels prepared loaded with celecoxib

| | | | Standard | | | |
|---|---|---|---|---|---|---|
| | | Actual | Deviation | | | |
| Sample | | CXB % | of Actual | | | Mass |
| No. | Sample name | (w/v) | CXB % | pH | Replicate | (mg) |
| 168-1 | 120 mb; hyd; | 9.5 | 0.8 | 6.78 | A | 49.78 |
| | 27.8% cxbst; | | | | B | 54.35 |
| | 5% SFf; 10% | | | | C | 53.45 |
| | CXBf; 40% | | | | | |
| | PEG4kf | | | | | |
| 168-2 | 120 mb; hyd; | 9.5 | 0.3 | 6.82 | A | 52.89 |
| | 27.8% cxbst; | | | | B | 54.44 |
| | 3% SFf; 10% | | | | C | 50.48 |
| | CXBf; 40% | | | | | |
| | PEG4kf | | | | | |
| 168-3 | 120 mb; hyd; | 11.9 | 3.5 | 7.1 | A | 56.07 |
| | 27.8% cxbst; | | | | B | 53.96 |
| | 5% SFf; 10% | | | | C | 49.44 |
| | CXBf, 10% | | | | | |
| | P188f | | | | | |
| 168-4 | 120 mb; hyd; | 9.6 | 0.8 | 7.06 | A | 50.42 |
| | 27.8% cxbst; | | | | B | 54.12 |
| | 3% SFf; 10% | | | | C | 50.14 |
| | CXBf; 10% | | | | | |
| | P188f | | | | | |
| 168-5 | 480 mb; hyd; | 9.3 | 0 | 7.15 | A | 51.75 |
| | 27.8% cxbst; | | | | B | 49.55 |
| | 5% SFf; 10% | | | | C | 55.33 |
| | CXBf; 40% | | | | | |
| | PEG4kf | | | | | |
| 168-6 | 480 mb; hyd; | 9.2 | 0.7 | 6.98 | A | 56.38 |
| | 27.8% cxbst; | | | | B | 50.92 |
| | 3% SFf; 10% | | | | C | 49.08 |
| | CXBf; 40% | | | | | |
| | PEG4kf | | | | | |
| 168-7 | 480 mb; hyd; | 8.7 | 0.1 | 7.16 | A | 55.12 |
| | 27.8% cxbst; | | | | B | 51.59 |
| | 5% SFf; 10% | | | | C | 54.18 |
| | CXBf; 10% | | | | | |
| | P188f | | | | | |
| 168-8 | 480 mb; hyd; | 9.8 | 0.6 | 7.15 | A | 55.9 |
| | 27.8% cxbst; | | | | B | 53.53 |
| | 3% SFf; 10% | | | | C | 56.17 |
| | CXBf; 10% | | | | | |
| | P188f | | | | | |
| 168-9 | 480 mb; hyd; | 9.4 | 1.6 | 7.13 | A | 52.39 |
| | 27.8% cxbst; | | | | B | 54.56 |
| | 2% SFf; 10% | | | | C | 53.38 |
| | CXBf; 10% | | | | | |
| | P188f | | | | | |

### In Vitro Release Experiments

In triplicate, 50 mg of each formulation was weighed into half of a #4 gelatin capsule (MyHerbar Dallas Tex.). It had previously been shown that the solubility of celecoxib in this release media was 850 μg/mL. 45 mL of this release media allowed for 38 mg CXB solubility. This media ensured sink conditions (greater than or equal to 5 times the CXB solubility) were maintained throughout the course of the study. The tubes were capped and incubated at 37° C. with shaking. 1 mL of the release media was collected from each sample at each timepoint and replaced with 1 mL fresh media. At each timepoint, the tubes were left to stand on end for at least 30 minutes to allow the formulation to settle prior to taking the sample. Release media was analyzed by HPLC-UV (Agilent 1290 HPLC system) at 260 nm. Controls were prepared at Day 0 by weighing 50 mg of each formulation in triplicate in separate 20 mL glass vials. Methanol was added to each sample to extract CXB. Samples were placed on a shaker at room temperature for 24 hours. The supernatant was analyzed by HPLC-UV to determine CXB loading. The results of the in vitro release experiments, seen in Table 14A, and Table 14B were consistent with first-order kinetics, with initial bursts from 25%-100%. All tested hydrogel formulations released the small molecule up to one month after the start of the experiment.

TABLE 14A
In vitro release kinetics for hydrogels loaded with celecoxib;
average cumulative percentage of API released

| Time | Sample No. | | | | | | | | |
|---|---|---|---|---|---|---|---|---|---|
| (days) | 168-1 | 168-2 | 168-3 | 168-4 | 168-5 | 168-6 | 168-7 | 168-8 | 168-9 |
| 0 | 0.00 | 0.00 | 0.00 | 0.00 | 0.00 | 0.00 | 0.00 | 0.00 | 0.00 |
| 1 | 24.59 | 42.53 | 51.15 | 42.75 | 30.64 | 96.24 | 43.26 | 27.68 | 46.90 |
| 4 | 50.91 | 67.96 | 73.54 | 69.47 | 56.39 | 96.99 | 73.38 | 51.56 | 74.89 |
| 7 | 67.59 | 81.51 | 79.05 | 82.51 | 75.78 | 96.32 | 87.00 | 66.01 | 87.51 |
| 14 | 79.43 | 86.60 | 75.51 | 88.35 | 86.61 | 94.02 | 94.64 | 80.88 | 93.81 |
| 25 | 96.86 | 98.22 | 85.72 | 104.14 | 102.82 | 105.89 | 110.51 | 100.24 | 105.61 |
| 29 | 95.90 | 96.43 | 82.39 | 99.86 | 102.21 | 100.32 | 105.56 | 95.38 | 102.51 |

TABLE 14B
Standard deviations of the average cumulative percentage
of API released from the in vitro release kinetics
experiments for hydrogels loaded with celecoxib

| Time | Sample No. | | | | | | | | |
|---|---|---|---|---|---|---|---|---|---|
| (days) | 168-1 | 168-2 | 168-3 | 168-4 | 168-5 | 168-6 | 168-7 | 168-8 | 168-9 |
| 0 | 0.00 | 0.00 | 0.00 | 0.00 | 0.00 | 0.00 | 0.00 | 0.00 | 0.00 |
| 1 | 3.04 | 7.33 | 2.96 | 13.35 | 3.38 | 1.36 | 15.21 | 5.30 | 10.79 |
| 4 | 5.07 | 5.88 | 1.77 | 11.33 | 5.30 | 2.46 | 19.62 | 6.69 | 9.42 |
| 7 | 5.21 | 4.57 | 4.00 | 9.15 | 10.63 | 2.75 | 16.71 | 6.27 | 8.46 |
| 14 | 4.56 | 2.18 | 7.29 | 4.63 | 5.20 | 3.93 | 11.75 | 4.33 | 1.02 |
| 25 | 2.68 | 4.97 | 9.73 | 8.47 | 4.63 | 0.74 | 6.66 | 4.63 | 3.76 |
| 29 | 1.80 | 3.88 | 8.30 | 4.87 | 3.02 | 2.68 | 7.77 | 2.59 | 2.74 |

For the hydrogels prepared with P188, the initial burst was the highest for the hydrogel with 5% (w/v) high molecular weight silk fibroin, as seen in Table 14A. The hydrogel with 3% (w/v) low molecular weight silk fibroin had the lowest initial burst of therapeutic agent. The remaining hydrogels had initial bursts of a similar magnitude, the values of which were between those of the 5% (w/v) high molecular weight and the 3% (w/v) low molecular weight silk fibroin hydrogels. The hydrogels (with P188) with higher concentrations of silk fibroin demonstrated greater initial bursts of API in comparison with the corresponding hydrogels with lower concentrations of silk fibroin. In addition, the hydrogels (with P188) prepared from higher molecular weight silk fibroin also demonstrated greater initial bursts of API than the corresponding hydrogels with lower molecular weight silk fibroin.

For the hydrogels prepared with PEG4k, the initial burst was the highest for the hydrogel prepared with 3% (w/v) low molecular weight silk fibroin, followed by the hydrogel prepared with 3% (w/v) high molecular weight silk fibroin. The hydrogel prepared the with 5% (w/v) high molecular weight silk fibroin had the lowest initial burst, as seen in Table 14A. The hydrogels (with PEG 4k) prepared from higher molecular weight silk fibroin demonstrated lower initial bursts of API than the hydrogels prepared from lower molecular weight silk fibroin. In addition, the hydrogel (with PEG4k) with a lower concentration of silk fibroin demonstrated a greater initial burst of API than the corresponding hydrogel with a higher concentration of silk fibroin.

The use of excipients with different molecular weights also revealed a pattern in the initial burst of therapeutic agent from the hydrogels. While both hydrogels were prepared at the same osmolarity, excipients used had different molecular weights. PEG4k had a molecular weight of 4 kDa, while P188 had a molecular weight of 8.4 kDa. The molecular weight of the excipient modulated the observed trends in the initial burst percentages. Hydrogels prepared from excipients with higher molecular weights demonstrated a direct relationship between the concentration of silk fibroin and the initial burst and a direct relationship between the molecular weight of the silk fibroin and the initial burst. Meanwhile, hydrogels prepared from excipients with lower molecular weights demonstrated an inverse relationship between the concentration of silk fibroin and the initial burst and an inverse relationship between the molecular weight of the silk fibroin and the initial burst.

### Example 5. Biocompatibility of Silk Fibroin Rods and Hydrogels

Silk fibroin rods or silk fibroin hydrogels were formulated with a generic NSAID. The silk fibroin rods had a diameter of 430 μm and a length of 10 mm. Silk fibroin hydrogels were formulated with and without 100 mg/mL NSAID. The rods or hydrogels were administered to healthy rabbits as 100 μL injections in a 27-gauge needle. The rods were pre-loaded into sterile 21G, 1″ needles with pieces of 28G wire were pre-cut, sterilized and placed into the needle from the hub. The needle was placed (as described below) and the formulation was pushed into the intravitreal space, 2 mm posterior to the limbus using the length of 28G wire. The wire extended past the end of needle 3-4 mm to ensure full injection. A lid speculum was inserted into the rabbit's left eye lid. The conjunctiva was drenched with BSS solution from a sterile dropper (3-5 drops). 1-2 drops of betadine solution was applied allowing 30 seconds after administration. One additional drop of betadine solution was applied followed by injection of the formulation using a double-plane tunnel technique (the sclera was penetrated at 15°-30°, then the needle is repositioned to a 45-60 angle while the sclera was still engaged; the formulation was delivered and the needle removed at a 90° angle). Following injection, the central retinal artery was examined via indirect ophthalmoscopy to confirm perfusion and 1-2 drops of betadine solution were added to the conjunctiva prior to removal of the speculum. The silk fibroin compositions remained cohesive or in one piece in the intravitreal space. The subjects experienced normal intraocular pressures, no local inflammation, no hemorrhage, and no other complications. The silk fibroin rods and hydrogels were tolerated in the intravitreal space.

### Example 6. Tolerability Studies

The tolerability of silk fibroin solutions, hydrogels, and rods was monitored in rabbits, rats, and dogs. All materials studied were well-tolerated clinically. The hydrogel material was observed to integrate into tissue with minimal inflammation, which was consistent with a transient local foreign body reaction. No adverse reactions were noted.

### Example 7. Human Whole Blood Assay

Whole human blood was exposed to soluble silk fibroin for 24 hours at 37° C. and assessed for inflammation. Lipopolysaccharide (LPS) was used as a positive stimulator of the inflammatory marker TNF-α, in whole blood. The experiments were conducted in the presence and absence of LPS to determine whether any formulation constituent had the activity of potentiating a known inflammatory signal. Plasma was collected at the end of the experiment and analyzed by enzyme-linked immunosorbent assay (ELISA) for TNF-α (FIG. 3). The experiments were performed with blood from 5 donors (FIG. 3) and repeated with 2 additional donors. The silk fibroin did not increase the release of TNF-α, and other inflammatory markers such as PGE2. The results were consistent with multiple silk fibroin formats, such as silk fibroin with different molecular weights, hydrogels, 3D fibroin scaffolds, and hydrogel extracts. No signs of local sensitization were detected after extended exposure.

### Example 8. Measurements of Diameter, Density and In Vitro Experiments on 1 mm Celecoxib Loaded Silk Fibroin Rods

The diameter of the silk-fibroin rods was measured using digital calipers. The rods were cut to 1 cm lengths to standardize release, and the weights of the rods were recorded. The density of the rods was calculated for each formulation. As seen in Table 15, the experimental data revealed that the samples generated at each theoretical w/w % formed silk rods with a diameter slightly below 1 mm, the theoretical silk rod diameter. In addition, most of the samples yielded silk rods with a density near 1 g/mL In Table 15, “Std. Dev.” Refers to standard deviation.

TABLE 15
Observed diameter and density of 1 mm silk-fibroin rods

| | | | | Den- |
|---|---|---|---|---|
| | | | | sity |
| | | Diam- | Den- | Std. |
| Sample | | eter | sity | Dev. |
| No. | Sample Name | (mm) | (g/mL) | (g/mL) |
| 8-58-1 | 480 mb; 1 mm; 20% st, 50 mgsf; | 0.93 | 0.79 | 0.05 |
| | 150 mgcxb; lyo, 25% sf; 75% cxb | | | |
| 8-58-2 | 480 mb; 1 mm; 20% st; 50 mgsf; | 0.95 | 0.83 | 0.08 |
| | 200 mgcxb; lyo; 20% sf; 80% cxb | | | |
| 8-58-4 | 480 mb; 1 mm; 30% st; 75 mgsf; | 0.88 | 1.00 | 0.06 |
| | 150 mgcxb; lyo; 33.3% sf; 66.7% | | | |
| | cxb | | | |
| 8-58-5 | 480 mb; 1 mm; 30% st; 75 mgsf; | 0.92 | 1.09 | 0.14 |
| | 200 mgcxb; lyo; 27.3% sf; 72.2% | | | |
| | cxb | | | |
| 8-58-6 | 480 mb; 1 mm; 30% st; 75 mgsf; | 0.96 | 1.07 | 0.05 |
| | 250 mgcxb; lyo; 23% sf; 77% cxb | | | |
| 8-58-7 | 480 mb; 1 mm; 40% st; 100 mgsf; | 0.88 | 1.19 | 0.07 |
| | 150 mgcxb; lyo; 40% sf; 60% cxb | | | |
| 8-58-8 | 480 mb; 1 mm; 40% st; 100 mgsf; | 0.91 | 1.28 | 0.05 |
| | 200 mgcxb; lyo; 33.3% sf; 66.7% | | | |
| | cxb | | | |
| 8-58-9 | 480 mb; 1 mm, 40% st; 100 mgsf; | 0.92 | 1.30 | 0.11 |
| | 250 mgcxb; lyo; 28.6% sf; 71.4% | | | |
| | cxb | | | |

Extraction controls were run to determine celecoxib (CXB) loading in the rods. Pre-weighed, 1 cm lengths of the rods were placed into 5 mL of 100% methanol, vortexed, and sonicated. The samples were left to shake overnight at room temperature. The methanol was then analyzed for CXB loading via UPLC. For most samples, the experimental loading percentage of CXB of the silk rods was lower than the theoretical loading percentage of CXB, as seen in Table 16. Many of the samples had actual CXB loadings around 8% lower than the theoretical CXB loading.

TABLE 16
Celecoxib loading after extraction for 1 mm rods

| | | Theoretical | Actual | Standard | |
|---|---|---|---|---|---|
| Sample | | CXB % | CXB % | Dev. of | Density |
| No. | Sample Name | (w/w) | (w/w) | CXB % | (g/mL) |
| 8-58-1 | 480 mb; 1 mm; 20% st; 50 mgsf; | 75 | 64.1 | 4.4 | 0.79 |
| | 150 mgcxb; lyo; 25% sf; 75% cxb | | | | |
| 8-58-2 | 480 mb; 1 mm; 20% st; 50 mgsf; | 80 | 70.4 | 0.8 | 0.83 |
| | 200 mgcxb; lyo; 20% sf; 80% cxb | | | | |
| 8-58-4 | 480 mb; 1 mm; 30% st; 75 mgsf; | 66.7 | 59.3 | 1.1 | 1.00 |
| | 150 mgcxb; lyo; 33.3% sf; 66.7% cxb | | | | |
| 8-58-5 | 480 mb; 1 mm; 30% st; 75 mgsf; | 72.2 | 65.6 | 4.1 | 1.09 |
| | 200 mgcxb; lyo; 27.3% sf; 72.2% cxb | | | | |
| 8-58-6 | 480 mb; 1 mm; 30% st; 75 mgsf; | 77 | 74.0 | 9.0 | 1.07 |
| | 250 mgcxb; lyo; 23% sf; 77% cxb | | | | |
| 8-58-7 | 480 mb; 1 mm; 40% st; 100 mgsf; | 60 | 59.7 | 15.7 | 1.19 |
| | 150 mgcxb; lyo; 40% sf; 60% cxb | | | | |
| 8-58-8 | 480 mb; 1 mm; 40% st; 100 mgsf; | 66.7 | 62.7 | 2.4 | 1.28 |
| | 200 mgcxb; lyo; 33.3% sf; 66.7% cxb | | | | |
| 8-58-9 | 480 mb; 1 mm; 40% st; 100 mgsf; | 71.4 | 65.9 | 4.5 | 1.30 |
| | 250 mgcxb; lyo; 28.6% sf; 71.4% cxb | | | | |

For the release experiments, the rods were placed into 45 mL of phosphate buffer, pH 7.4, 2% (v/v) Polysorbate-80 (from Croda, Snaith UK), and 0.05 (w/v) sodium azide (from Fisher Chemical, Waltham Mass.). This buffer ensured that the release was conducted under sink conditions (≥5× saturated solubility). The samples were incubated at 37° C. with gentle shaking. 1 mL of the release medium was taken at each timepoint (typically 1, 4, and 7 days and then weekly thereafter). The release medium was then analyzed via ultra-performance liquid chromatography (UPLC) to determine CXB concentration. The results were shown in Table 17A and Table 17B.

TABLE 17A
In vitro release kinetics of celecoxib from 1 mm silk-
fibroin rods; cumulative percentage of API released

| | Sample No. | | | | | | | |
|---|---|---|---|---|---|---|---|---|
| Day | 8-58-1 | 8-58-2 | 8-58-4 | 8-58-5 | 8-58-6 | 8-58-7 | 8-58-8 | 8-58-9 |
| 0 | 0 | 0.0 | 0.0 | 0 | 0.0 | 0.0 | 0.0 | 0.0 |
| 1 | 18.6 | 15.0 | 10.0 | 9.0 | 7.1 | 5.6 | 6.2 | 5.7 |
| 4 | 33.3 | 29.1 | 21.4 | 17.7 | 14.3 | 11.1 | 12.8 | 11.8 |
| 7 | 48.4 | 41.5 | 28.1 | 25.4 | 21.2 | 16.8 | 18.7 | 17.6 |
| 11 | 60.3 | 51.2 | 37.4 | 34.2 | 27.8 | 22.2 | 25.0 | 23.8 |
| 14 | 66.6 | 58.8 | 41.6 | 37.8 | 32.1 | 25.1 | 28.2 | 26.5 |
| 21 | 81.9 | 73.7 | 53.2 | 50.0 | 42.0 | 34.1 | 36.2 | 33.8 |
| 28 | 98.0 | 88.3 | 65.5 | 59.0 | 51.4 | 42.3 | 43.5 | 42.3 |
| 35 | 96.9 | 91.4 | 67.8 | 62.6 | 54.4 | 40.9 | 48.3 | 45.0 |
| 42 | 93.7 | 91.5 | 66.7 | 61.6 | 54.8 | 40.9 | 49.3 | 44.3 |
| 49 | 101.3 | 96.3 | 76.3 | 71.0 | 62.8 | 47.8 | 54.8 | 51.2 |
| 56 | 98.1 | 95.4 | 79.1 | 73.5 | 66.8 | 49.9 | 58.5 | 52.5 |
| 64 | 97.2 | 102.0 | 84.4 | 77.6 | 72.5 | 52.9 | 60.9 | 57.3 |
| 70 | — | — | 88.1 | 81.2 | 73.6 | 57.4 | 65.5 | 60.8 |
| 76 | — | — | 89.6 | 83.2 | 75.0 | 58.1 | 66.9 | 62.8 |
| 84 | — | — | 94.9 | 87.3 | 79.3 | 61.7 | 71.0 | 65.8 |
| 98 | — | — | 116.8 | 106.1 | 98.6 | 75.7 | 88.8 | 82.6 |
| 112 | — | — | 118.2 | 108.5 | 103.9 | 81.1 | 96.2 | 87.7 |
| 126 | — | — | 115.1 | 106.6 | 103.6 | 83.4 | 101.8 | 91.8 |
| 147 | — | — | — | — | — | 92.2 | 111.6 | 100.5 |
| 162 | — | — | — | — | — | 98.9 | 121.7 | 108.8 |
| 176 | — | — | — | — | — | 103.1 | 138.4 | 114.6 |
| 190 | — | — | — | — | — | 104.9 | 124.2 | 115.4 |
| 204 | — | — | — | — | — | 107.1 | 123.2 | 116.2 |

TABLE 17B
Standard deviations of the data from the in vitro release kinetics of celecoxib
from 1 mm silk-fibroin rods; cumulative percentage of API released

| | Sample No. | | | | | | | |
|---|---|---|---|---|---|---|---|---|
| Day | 8-58-1 | 8-58-2 | 8-58-4 | 8-58-5 | 8-58-6 | 8-58-7 | 8-58-8 | 8-58-9 |
| 0 | 0 | 0.00 | 0.00 | 0 | 0.00 | 0.00 | 0.00 | 0.00 |
| 1 | 3.7 | 1.94 | 0.42 | 0.9 | 0.32 | 0.16 | 0.05 | 0.30 |
| 4 | 4.3 | 2.80 | 3.95 | 0.5 | 0.19 | 0.24 | 0.54 | 0.61 |
| 7 | 5.7 | 3.31 | 1.22 | 0.5 | 0.22 | 0.40 | 0.32 | 0.75 |
| 11 | 6.9 | 2.87 | 0.58 | 0.7 | 0.49 | 0.80 | 0.90 | 0.74 |
| 14 | 6.3 | 3.33 | 0.98 | 0.7 | 0.94 | 0.64 | 0.77 | 0.80 |
| 21 | 6.8 | 4.21 | 3.11 | 6.0 | 2.14 | 4.44 | 0.98 | 2.17 |
| 28 | 6.3 | 4.11 | 0.97 | 1.2 | 1.31 | 2.23 | 2.76 | 1.84 |
| 35 | 6.3 | 2.93 | 1.40 | 1.5 | 1.98 | 1.74 | 3.05 | 1.79 |
| 42 | 3.46 | 7.23 | 2.09 | 1.6 | 2.20 | 1.43 | 2.84 | 0.94 |
| 49 | 2.91 | 3.96 | 2.62 | 1.2 | 1.93 | 1.69 | 1.86 | 1.61 |
| 56 | 2.18 | 5.20 | 2.95 | 1.0 | 3.00 | 2.08 | 2.12 | 1.43 |
| 64 | 5.31 | 7.87 | 8.65 | 1.1 | 7.93 | 3.39 | 1.80 | 1.47 |
| 70 | — | — | 2.85 | 1.6 | 2.52 | 2.28 | 3.04 | 2.64 |
| 76 | — | — | 3.28 | 0.8 | 1.93 | 3.36 | 3.57 | 3.01 |
| 84 | — | — | 3.93 | 1.9 | 2.43 | 3.68 | 4.64 | 3.83 |
| 98 | — | — | 6.36 | 0.8 | 3.45 | 4.39 | 7.17 | 4.62 |
| 112 | — | — | 8.59 | 1.5 | 3.88 | 5.16 | 7.89 | 4.77 |
| 126 | — | — | 7.38 | 1.3 | 3.87 | 6.56 | 11.24 | 5.32 |
| 147 | — | — | — | — | — | 8.93 | 16.8 | 8.89 |
| 162 | — | — | — | — | — | 9.82 | 20.4 | 7.60 |
| 176 | — | — | — | — | — | 10.41 | 12.5 | 8.53 |
| 190 | — | — | — | — | — | 9.85 | 16.7 | 10.83 |
| 204 | — | — | — | — | — | 9.42 | 10.6 | 9.36 |

The data demonstrated near-zero-order release kinetics. Each silk fibroin rod sample experienced an initial burst of API release as seen in Table 18, followed by the continued gradual release of the therapeutic agent at a slower rate. The initial burst of API release from the rods ranged from about 5-20% of the API loaded into the rods by mass. The theoretical loading percentage of CXB affected the initial burst of API release. Higher percentages of silk fibroin in the theoretical loading (w/w) percentages of silk fibroin correlated with lower initial burst rates. This inverse relationship between the amount of silk fibroin in the rods and the initial burst rate was evident across all samples. Sample 8-58-1 reached complete release by day 35, and 8-58-2 reached completion by day 64. Samples 8-58-4 and 8-58-5, reached complete release by day 98. Sample 8-58-6 reached complete release by day 112.

TABLE 18
Celecoxib release rates for 1 mm rods

| | | | | Daily | | |
|---|---|---|---|---|---|---|
| | Ratio | | | Release | Initial | |
| | CXB:SF | Ratio | Initial | % (at | burst:Daily | Den- |
| Sample | Theo- | CXB:SF | Burst | 64 | release (at | sity |
| No. | retical | Actual | % | days) | 64 days) | (g/mL) |
| 8-58-1 | 3.0 | 1.8 | 18.6 | 1.34 | 13.9 | 0.79 |
| 8-58-2 | 4.0 | 2.4 | 15.0 | 1.42 | 10.6 | 0.83 |
| 8-58-4 | 2.0 | 1.5 | 10.0 | 1.19 | 8.4 | 1.00 |
| 8-58-5 | 2.6 | 1.9 | 9.0 | 1.11 | 8.1 | 1.09 |
| 8-58-6 | 3.3 | 2.8 | 7.1 | 1.04 | 6.8 | 1.07 |
| 8-58-7 | 1.5 | 1.5 | 5.6 | 0.76 | 7.4 | 1.19 |
| 8-58-8 | 2.0 | 1.7 | 6.2 | 0.90 | 6.9 | 1.28 |
| 8-58-9 | 2.5 | 1.9 | 5.7 | 0.82 | 6.9 | 1.30 |

The kinetics data demonstrated the possible existence of a relationship between the rate of API release and the (w/w) ratio of API to silk fibroin for the 1 mm silk fibroin rods. These ratios were calculated for both the theoretical loading and the actual loading of the rods. The use of each formulation in a device or product might depend on the desired amount of API released in the time frame of interest. For example, if a smaller amount of the API needed to be released in the designated time frame, the formulations from Samples 8-58-7 through 8-58-9 would be most effective. As seen in Table 17A and Table 18, the release duration of CXB was related to the rod density, with increased density resulting in longer release times and slower release rates. The rods with a higher density also demonstrated a lower daily release percentage and lower initial burst percentages. Daily release percentage was defined as the weight percent of the total API released per day, and it was calculated as the slope of the plot of cumulative release over time. We have shown the daily release percentages calculated for the first 64 days of the study. The rod density was tuned by varying the starting concentration of the silk-fibroin used during formulation. For example, the formulations prepared with 40% (w/v) silk-fibroin solution had the highest densities of 1.30, 1.28, and 1.19 g/mL, while the formulations prepared with 20% (w/v) silk-fibroin had the lowest densities of 0.83 and 0.79 g/mL. The initial burst and release rate decreased with increasing density. Ultimately, the samples with a density below 1.0 g/mL reached complete release about 64 days or less, the samples with a density between 1.0 g/mL and 1.1 g/mL reached complete release in about 98 days, and the samples with a density above 1.1 g/mL reached complete release in greater than 98 days. The higher density rods represented a more tightly packed CXB/fibroin formulation. Since both the CXB as well as the formulated silk-fibroin were hydrophobic, this lead to the prevention of water uptake into the rod. The more tightly packed rods also slowed the diffusion of CXB from the formulation by crating locally saturated regions of CXB within the rod, slowing the dissolution and release.

### Example 9. Measurements of Diameter, Density and In Vitro Experiments on 0.5 mm Celecoxib Loaded Silk Fibroin Rods

As seen in the experiments on the 1 mm silk rods, the diameter of the 0.5 mm silk-fibroin rods was measured using digital calipers. The rods were cut to 1 cm lengths to standardize release, and the weights of the rods were recorded. The densities of the rods were calculated for each formulation. The rods were placed into 45 mL of 1× phosphate buffer, pH 7.4, 0.3% (v/v) Polysorbate-80 (from Croda, Snaith UK), and 0.05% (w/v) sodium azide (from Fisher Chemical, Waltham Mass.). This buffer ensured that the release was conducted under sink conditions (≥5× saturated solubility). A suspension of celecoxib (CXB) (from Cipla. Miami Fla.) containing 800 μg CXB was used as a control. The samples were incubated at 37° C. with gentle shaking. 1 mL of the release medium was taken at each timepoint (typically 1, 4, and 7 days and then weekly thereafter). The release medium was then analyzed via UPLC at 260 nm to determine CXB concentration. The data from the experiment was summarized in Table 19. Extraction controls were run to determine CXB loading in the rods. Pre-weighed, 1 cm lengths of the rods were placed into 2 mL of 100% methanol, vortexed, and sonicated. The samples were left to shake overnight at room temperature. The methanol was then analyzed for CXB loading via HPLC.

TABLE 19
Precise diameter, density, and loading percentages of 0.5 mm silk-fibroin rods
(480 mb; 0.5 mm; 40% st; 100 mgsf; 200 mgcxb; lyo; 33.3% sf; 66.7% cxb)

| | Ratio | Ratio | | | Theoretical | Actual | Standard |
|---|---|---|---|---|---|---|---|
| Sample | CXB:SF | CXB:SF | Diameter | Density | CXB % | CXB % | Dev. of |
| No. | Theoretical | Actual | (mm) | (g/mL) | (w/w) | (w/w) | CXB % |
| 8-65-6 | 2 | 1.0 | 0.43 | 1.2 | 66.7 | 48.9 | 3.2 |

The release of CXB was monitored as described over a period of 77 days, as seen in Table 20 and Table 21. The data demonstrated near-zero-order release kinetics. The CXB suspension was completely released after 1 day. The rod formulation, however, displayed very extended release. The initial burst from the rod was only 12.9% with near zero-order release out to 21 days. After 21 days, the release rate slowed even more, allowing for a second zero-order segment of release out to completion at about 70 days. After day 70, no additional API was released. In Table 20, “Std. Dev.” refers to standard deviation.

TABLE 20
In vitro release kinetics of celecoxib from 0.5 mm silk-
fibroin rods; average cumulative percentage of API released

Average Cumulative % Released

| | CXB | Std. Dev. | | 8-65-6 |
|---|---|---|---|---|
| Day | Suspension | suspension | 8-65-6 | Std. Dev. |
| 0 | 0 | 0 | 0 | 0 |
| 1 | 111.8 | 0.8 | 12.9 | 1.3 |
| 2 | 110.9 | 0.7 | 18.7 | 1.3 |
| 7 | 113.8 | 0.8 | 43.7 | 2.4 |
| 10 | — | — | 57.9 | 2.7 |
| 14 | — | — | 65.4 | 2.9 |
| 21 | — | — | 84.7 | 4.9 |
| 28 | — | — | 92.5 | 5.3 |
| 35 | — | — | 95.1 | 5.4 |
| 42 | — | — | 102.1 | 5.5 |
| 49 | — | — | 113.9 | 5.9 |
| 56 | — | — | 118.9 | 5.6 |
| 63 | — | — | 124.0 | 5.0 |
| 70 | — | — | 117.8 | 4.7 |
| 77 | — | — | 117.2 | 4.4 |

TABLE 21
Daily percentage of celecoxib released for rods of different diameters

| | | | | Initial | Measured |
|---|---|---|---|---|---|
| Sample | | Initial | Daily % | burst:Daily | rod diameter |
| No. | Sample Name | Burst % | Released | release | (mm) |
| 8-65-6 | 480 mb; 0.5 mm; 40% st; | 12.9 | 1.8 | 7.2 | 0.43 |
| | 100 mgsf; 200 mgcxb; | | | | |
| | lyo; 33.3% sf; 66.7% cxb | | | | |
| 8-58-8 | 480 mb; 1 mm; 40% st; | 6.2 | 0.9 | 6.9 | 0.91 |
| | 100 mgsf; 200 mgcxb; | | | | |
| | lyo; 33.3% sf; 66.7% cxb | | | | |

The data from this experiment suggested that the rate of release of therapeutic, CXB, was inversely related to the diameter of the silk rods. The daily release percentage of CXB, as well as the ratio of the initial burst to the daily release percentage and other rod parameters, is shown in Table 21. The daily percentage of CXB released for sample 8-65-6, which was calculated for 63 days, was 1.8%. The corresponding 1 mm silk rods (Sample 8-58-8), as seen in 1 mm silk rod experiments, were 33.3% (w/w) SF, 66.7% (w/w) CXB, and had a 480-minute boil. These 1 mm silk rods released 0.9%/of the loaded CXB per day. The almost two-fold difference between the 1 mm and 0.5 mm silk rods suggested that the therapeutics were released more quickly from rods with a smaller diameter. This difference was also observed in the initial burst of drug release, however, the ratio between the initial burst and the daily release percentage remained consistent regardless of rod diameter. The 1 mm silk rods had an initial burst of 6.2%, while the corresponding 0.5 mm rods had an initial burst of 12.9%. The changes in initial burst and daily release percentage were likely due to the greater surface area to volume ratio in the rods of smaller diameter. In the narrower rods, water penetration and diffusion lengths were shorter, which lead to the faster releasing effect. These narrower rods could be injected through a 21-22G needle (standard for intravitreal injection devices), making them appropriate for intraocular delivery.

It should be noted that the actual CXB loading of 1 mm rods was higher than that of the 0.5 mm silk rods. This higher loading could alter the rate of CXB release between rods of the same theoretical formulation. Furthermore, the experiments for the 1 mm silk rods were carried out over a period of 126 days, which was longer than the experiments for the 0.5 mm rods. The release of CXB may decrease over longer periods of time, and the potential change in rate over time may alter the average daily percentage released.

### Example 10. Comparison of Silk Fibroin Rods Prepared Via Lyophilization Vs Oven Drying

The silk yarn was purchased from Jiangsu SOHO International Group (Jiangsu, China). Lithium bromide and phosphate buffer saline were purchased from Sigma Aldrich (St. Louis, Mo.). The potassium phosphate monobasic and potassium phosphate dibasic were purchased from Sigma Aldrich Fine Chemicals (SAFC) (St. Louis, Mo.). The sodium carbonate and the sodium azide were purchased from Fisher Chemical (Waltham, Mass.). The celecoxib (CXB) was purchased from Cipla (Miami, Fla.).

### Silk Fibroin Isolation

The silk yarn was degummed at 100° C. for either 120 or 480 minutes in 0.02 M sodium carbonate solution to remove sericin and modify the molecular weight. The total boiling time was discussed in terms of minute boil, or “mb.” Longer boiling times produced silk fibroin with lower average molecular weights. The objective of this experiment was to determine any difference in the release rate of the API between the silk rods prepared via lyophilization and the silk rods prepared via oven drying. Silk-fibroin (Jiangsu SOHO) was isolated as described in the preparation of the silk fibroin rods with no additives. Briefly, Silk yarn, purchased from Jiangsu SOHO International Group, was degummed to remove sericin. 30 grams of cut silk yarn were boiled at 100° C. in 3 L of deionized (DI) water with 0.02 M sodium carbonate with stirring. The yarn was then transferred to a new boiling 0.02 M sodium carbonate aqueous solution and boiled at 100° C. for additional time with stirring. The fibroin was then placed in DI water at 60-70° C. for 20 minutes with stirring, and then rinsed with clean DI water. This process was repeated 3 times. The fibroin was placed in clean D water, stirred for 20 minutes, then rinsed with clean D water, and this process was repeated for a total of 3×20 min.-rinse cycles.

The fibroin was dried overnight, weighed, and dissolved at 20% (w/v) in a 9.3 M aqueous solution of lithium bromide (from Sigma-Aldrich, St. Louis, Mo.) for 5 hours at 60° C. The resulting fibroin solution was dialyzed against water at 4° C. in a 50 kDa regenerated cellulose dialysis tubing for 48 hours, with 6 water changes to remove the excess salt. The conductivity was recorded after each water change with a digital quality tester. When the conductivity was under 5 ppm, the fibroin solution was determined to be ready. The solution was then centrifuged for 20 minutes at 9,000 RPM and 4° C. to remove insoluble particles. Solutions were diluted to a final concentration of 3% (w/v) in 10 mM phosphate buffer, pH 7.4, filtered through a 0.22 μm filter, frozen in liquid nitrogen, and lyophilized for 72 hours.

### Silk Fibroin Rod Preparation

Lyophilized silk fibroin was reconstituted to either 20, 30, or 40% (w/v) with DI water. The desired amount of CXB was weighed into 4 mL glass vials. 250 μL of stock fibroin solution was then added to each vial accordingly. The fibroin and CXB was mixed both manually using a spatula and with a vortex. This mixture was then transferred to a 1 mL syringe using the spatula and extruded into 2×10 cm lengths of 500 μm ID polytetrafluoroethylene (PTFE) tubing (from Van Waters and Rogers (VWR), PA. USA). The tubing was then sealed on both ends using Parafilm and incubated at 37° C. to induce gelation. The lengths of tubing were cut into 2 cm sections. Half of the sections were dried for 48 hours in an oven at 60° C. The other half were frozen at −80° C. and lyophilized. Rods were stored at 4° C. prior to use.

The samples, shown in Table 22, are named by the process used to prepare and formulate each silk rod. For example, the sample named “480 mb; 0.5 mm; 40% st; 100mgsf; 100mgcxb; lyo; 50% sf; 50% cxb” refers to a silk fibroin rod prepared from silk degummed with a 480-minute boil, an extrusion with a 0.5 mm diameter, a preparation from a 40% stock solution of silk fibroin, a preparation from 100 mg of silk fibroin, a preparation from 100 mg of celecoxib, lyophilization, a theoretical w/w percentage of 50% silk fibroin, and a theoretical w/w percentage of 50% celecoxib. Samples prepared via oven drying were labeled with “oven”. Some samples were prepared with silk fibroin degummed with a 120-minute boil (120 mb). The final rods contained trace amounts of potassium phosphate buffer (with potassium phosphate dibasic and potassium phosphate buffer monobasic). In Table 22, “Std. Dev.” refers to standard deviation.

TABLE 22
Theoretical and experimental loading percentages for oven-dried
and lyophilized 0.5 mm silk-fibroin rods

| | | | | Actual | | Std. | Silk | |
|---|---|---|---|---|---|---|---|---|
| | | Stock Conc. | | Silk- | Actual | Dev. of | Prep | Phosphate |
| | | of Silk For | | Fibroin | CXB | CXB | Boil | Buffer |
| Sample | Sample | Formulation | Drying | Final % | Final % | Final % | Time | Conc. |
| No. | Name | (w/v %) | Method | (w/w) | (w/w) | (w/w) | (min) | (mM) |
| 177-1A | 480 mb; 0.5 mm; | 40 | Lyophilized | 62.30 | 37.70 | 0.52 | 480 | 95.2 |
| | 40% st; | | | | | | | |
| | 100 mgsf; | | | | | | | |
| | 100 mgcxb; lyo; | | | | | | | |
| | 50% sf; 50% cxb | | | | | | | |
| 177-1B | 480 mb; 0.5 mm; | 40 | Oven | 61.54 | 38.46 | 0.08 | 480 | 95.2 |
| | 40% st; | | | | | | | |
| | 100 mgsf; | | | | | | | |
| | 100 mgcxb; | | | | | | | |
| | oven; 50% sf; | | | | | | | |
| | 50% cxb | | | | | | | |
| 177-2A | 480 mb; 0.5 mm | 40 | Lyophilized | 53.14 | 46.86 | 0.70 | 480 | 83.3 |
| | 40% st; | | | | | | | |
| | 100 mgsf; | | | | | | | |
| | 150 mgcxb; lyo; | | | | | | | |
| | 40% sf; 60% cxb | | | | | | | |
| 177-2B | 480 mb; 0.5 mm; | 40 | Oven | 53.27 | 46.73 | 1.19 | 480 | 83.3 |
| | 40% st; | | | | | | | |
| | 100 mgsf; | | | | | | | |
| | 150 mgcxb; | | | | | | | |
| | oven; 40% sf; | | | | | | | |
| | 60% cxb | | | | | | | |
| 177-4A | 480 mb; 0.5 mm; | 40 | Lyophilized | 45.61 | 54.39 | 0.92 | 480 | 66.7 |
| | 40% st; | | | | | | | |
| | 100 mgsf; | | | | | | | |
| | 250 mgcxb; lyo; | | | | | | | |
| | 28.6% sf; | | | | | | | |
| | 71.4% cxb | | | | | | | |
| 177-4B | 480 mb; 0.5 mm; | 40 | Oven | 45.72 | 54.28 | 0.93 | 480 | 66.7 |
| | 40% st; | | | | | | | |
| | 100 mgsf; | | | | | | | |
| | 250 mgcxb; | | | | | | | |
| | oven; 28.6% sf; | | | | | | | |
| | 71.4% cxb | | | | | | | |
| 177-6A | 480 mb; 0.5 mm; | 30 | Lyophilized | 46.03 | 53.97 | 1.82 | 480 | 55.6 |
| | 30% st; 75 mgsf; | | | | | | | |
| | 200 mgcxb; lyo; | | | | | | | |
| | 27.3% sf; | | | | | | | |
| | 72.7% cxb | | | | | | | |
| 177-6B | 480 mb; 0.5 mm; | 30 | Oven | 45.27 | 54.73 | 1.01 | 480 | 55.6 |
| | 30% st; 75 mgsf; | | | | | | | |
| | 200 mgcxb; | | | | | | | |
| | oven; 27.3% sf; | | | | | | | |
| | 72.7% cxb | | | | | | | |
| 177-7A | 120 mb; 0.5 mm; | 20 | Lyophilized | 43.35 | 56.65 | 2.97 | 120 | 37 |
| | 20% st; 50mgsf; | | | | | | | |
| | 200 mgcxb; lyo; | | | | | | | |
| | 20% sf; 80% cxb | | | | | | | |
| 177-7B | 120 mb; 0.5 mm; | 20 | Oven | 39.71 | 60.29 | 0.26 | 120 | 37 |
| | 20% st; 50 mgsf; | | | | | | | |
| | 200 mgcxb; | | | | | | | |
| | oven; 20% sf; | | | | | | | |
| | 80% cxb | | | | | | | |
| 177-8A | 120 mb; 0.5 mm; | 30 | Lyophilized | 42.23 | 57.77 | 4.08 | 120 | 55.6 |
| | 30% st; 75 mgsf; | | | | | | | |
| | 200 mgcxb; lyo; | | | | | | | |
| | 27.3% sf; | | | | | | | |
| | 72.7% cxb | | | | | | | |
| 177-8A | 120 mb; 0.5 mm; | 30 | Oven | 42.25 | 57.75 | 3.87 | 120 | 55.6 |
| | 30% st; 75 mgsf; | | | | | | | |
| | 200 mgcxb; | | | | | | | |
| | oven; 27.3% sf; | | | | | | | |
| | 72.7% cxb | | | | | | | |
| 177-9A | 120 mb; 0.5 mm | 40 | Lyophilized | 48.46 | 51.54 | 0.48 | 120 | 74.1 |
| | 40% st; | | | | | | | |
| | 100 mgsf; | | | | | | | |
| | 200 mgcxb; lyo; | | | | | | | |
| | 33.3% sf; | | | | | | | |
| | 66.7% cxb | | | | | | | |
| 177-9B | 120 mb 0.5 mm; | 40 | Oven | 48.93 | 51.07 | 3.46 | 120 | 74.1 |
| | 40% st; | | | | | | | |
| | 100 mgsf; | | | | | | | |
| | 200 mgcxb; | | | | | | | |
| | oven; 33.3% sf; | | | | | | | |
| | 66.7% cxb | | | | | | | |

### In Vitro Release

The rods were cut to 1 cm lengths to standardize release, and the weights of the rods were recorded. In triplicate, a 1 cm segment of rod was weighed into a 50-mL conical tube. 45 mL of release medium (phosphate buffered saline, 0.3% Polysorbate-80, and 0.05% sodium azide) was added to each tube. We had previously shown that this media would ensure sink conditions (≥5×CXB solubility) are maintained throughout the study. The tubes were incubated at 37° C. with shaking. 1 mL of the release media was collected from each sample at days 1, 4, 7, 10, 14, and weekly thereafter and replaced with fresh media. Release media was analyzed for CXB concentration by HPLC-UV at 260 nm.

Controls were prepared by weighing 1 cm of each formulation in triplicate in separate glass vials. Methanol was added to each vial. Samples were vortexed, sonicated, and placed on a shaker at room temperature for 24 hours. The supernatant was analyzed by HPLC to determine CXB loading (mg/g) as seen in Table 22. CXB loaded silk-fibroin rods were prepared with loadings ranging from 38-60% (w/w). Drying method did not have an impact on the drug loading, suggesting that the drug was stable through the 60° C. treatment. The release kinetics of both the lyophilized and the oven dried silk rods were shown in Table 23A, Table 23B, and Table 24. All samples showed zero percent (%) API release on day zero. The rods demonstrated near zero-order kinetics of API rlease.

TABLE 23A
In vitro release kinetics of celecoxib from 0.5 mm silk-fibroin rods, lyophilized
vs. oven dried; average cumulative percentage of API released

| Lot | Day | | | | | | | | | | | |
|---|---|---|---|---|---|---|---|---|---|---|---|---|
| No. | 1 | 3 | 7 | 10 | 14 | 21 | 28 | 35 | 42 | 49 | 56 | 63 |
| 177- | 13.6 | 27.8 | 51.5 | 58.9 | 70.5 | 80.2 | 92.2 | 105.9 | 112.3 | 110.7 | 114.8 | — |
| 1A | | | | | | | | | | | | |
| 177- | 10.1 | 21.6 | 41.0 | 47.4 | 57.2 | 66.3 | 77.3 | 90.3 | 97.6 | 99.8 | 108.9 | 112.2 |
| 1B | | | | | | | | | | | | |
| 177- | 15.4 | 32.2 | 59.6 | 66.9 | 78.7 | 87.9 | 97.3 | 105.9 | 108.0 | 104.8 | — | — |
| 2A | | | | | | | | | | | | |
| 177- | 12.1 | 25.4 | 47.4 | 54.7 | 64.8 | 73.6 | 83.5 | 95.0 | 100.6 | 99.9 | 106.0 | — |
| 2B | | | | | | | | | | | | |
| 177- | 13.0 | 25.8 | 50.0 | 55.7 | 66.4 | 75.3 | 87.5 | 97.8 | 106.9 | 106.7 | 110.7 | — |
| 4A | | | | | | | | | | | | |
| 177- | 14.2 | 30.5 | 57.6 | 65.2 | 76.9 | 85.9 | 96.1 | 108.1 | 112.4 | 108.2 | — | — |
| 4B | | | | | | | | | | | | |
| 177- | 18.4 | 35.7 | 63.3 | 70.9 | 82.6 | 90.4 | 96.0 | 102.9 | 103.5 | 99.5 | — | — |
| 6A | | | | | | | | | | | | |
| 177- | 16.2 | 33.8 | 66.2 | 72.8 | 85.7 | 94.4 | 100.0 | 107.4 | 108.4 | 104.1 | — | — |
| 6B | | | | | | | | | | | | |
| 177- | 23.7 | 46.6 | 83.6 | 93.7 | 108.5 | 112.3 | 111.0 | — | — | — | — | — |
| 7A | | | | | | | | | | | | |
| 177- | 15.7 | 34.3 | 67.3 | 76.6 | 90.9 | 100.4 | 104.9 | 108.8 | 108.6 | 104.3 | — | — |
| 7B | | | | | | | | | | | | |
| 177- | 14.5 | 31.6 | 57.7 | 66.3 | 78.5 | 87.8 | 97.2 | 106.3 | 106.5 | 102.4 | — | — |
| 8A | | | | | | | | | | | | |
| 177- | 15.8 | 31.9 | 58.5 | 66.2 | 77.9 | 87.1 | 96.8 | 106.2 | 106.8 | 102.4 | — | — |
| 8B | | | | | | | | | | | | |
| 177- | 14.1 | 28.8 | 51.6 | 58.1 | 68.6 | 76.5 | 86.0 | 97.3 | 102.2 | 99.5 | — | — |
| 9A | | | | | | | | | | | | |
| 177- | 13.4 | 27.4 | 48.9 | 54.8 | 64.7 | 72.2 | 81.4 | 92.5 | 97.9 | 97.6 | 101.9 | — |
| 9B | | | | | | | | | | | | |
| 177- | 106.2 | 106.2 | 110.9 | — | — | — | — | — | — | — | — | — |
| 10 | | | | | | | | | | | | |

TABLE 23B
Standard deviation of the In vitro release kinetics of celecoxib from 0.5 mm silk-
fibroin rods, lyophilized vs. oven dried; cumulative percentage of API released

| Lot | Day | | | | | | | | | | | | |
|---|---|---|---|---|---|---|---|---|---|---|---|---|---|
| No. | 0 | 1 | 3 | 7 | 10 | 14 | 21 | 28 | 35 | 42 | 49 | 56 | 63 |
| 177- | 0 | 1.1 | 2.2 | 4.0 | 4.9 | 5.5 | 6.9 | 8.2 | 10.2 | 11.2 | 10.3 | 8.0 | — |
| 1A | | | | | | | | | | | | | |
| 177- | 0 | 0.5 | 1.2 | 1.7 | 2.0 | 2.1 | 2.3 | 2.6 | 4.2 | 4.3 | 4.1 | 4.2 | 4.1 |
| 1B | | | | | | | | | | | | | |
| 177- | 0 | 3.0 | 5.6 | 9.6 | 10.2 | 11.3 | 11.8 | 12.1 | 10.3 | 7.6 | 6.3 | — | — |
| 2A | | | | | | | | | | | | | |
| 177- | 0 | 2.6 | 3.9 | 6.2 | 6.9 | 7.4 | 7.8 | 8.0 | 7.8 | 5.7 | 2.7 | 1.0 | — |
| 2B | | | | | | | | | | | | | |

All CXB loaded rod formulations exhibited biphasic release. Initial zero-order release from 1-10 days and a second zero-order profile from 10 days to completion. The rods reached complete release between 14 and 56 days.

In many of the samples subjected to the 480 mb degumming process, the initial burst of API release, determined as the total w/w percentage of CXB released in one day, was smaller for the oven dried silk rods than the lyophilized silk rods. For many rods prepared under identical conditions except for drying, the oven dried rods released between 5 and 35% less API during the initial burst than their lyophilized counterparts. This difference, shown in Table 24 was determined as the percent error between the initial bursts of the oven dried and lyophilized rods prepared under otherwise identical conditions.

TABLE 24
Analysis of initial burst percentages of
oven dried and freeze dried 0.5 mm rods

| | | Initial burst | |
|---|---|---|---|
| | | (% API | Differ- |
| Sample | | released | ence |
| No. | Sample Name | by mass) | by % |
| 177-1A | 480 mb; 0.5 mm; 40% st; 100 mgsf; | 13.6 | 25.8 |
| | 100 mgcxb; lyo; 50% sf; 50% cxb | | |
| 177-1B | 480 mb; 0.5 mm; 40% st; 100 mgsf; | 10.1 | — |
| | 100 mgcxb; oven; 50% sf; 50% cxb | | |
| 177-2A | 480 mb; 0.5 mm; 40% st; 100 mgsf; | 15.4 | 21.4 |
| | 150 mgcxb; lyo; 40% sf; 60% cxb | | |
| 177-2B | 480 mb; 0.5 mm; 40% st; 100 mgsf; | 12.1 | — |
| | 150 mgcxb; oven; 40% sf; 60% cxb | | |
| 177-4A | 480 mb; 0.5 mm; 40% st; 100 mgsf; | 13.0 | −9.6 |
| | 250 mgcxb; lyo; 28.6% sf; 71.4% cxb | | |
| 177-4B | 480 mb; 0.5 mm; 40% st; 100 mgsf; | 14.2 | — |
| | 250 mgcxb; oven; 28.6% sf; 71.4% cxb | | |
| 177-6A | 480 mb; 0.5 mm; 30% st; 75 mgsf; | 18.4 | 12.0 |
| | 100 mgcxb; lyo; 27.3% sf; 72.7% cxb | | |
| 177-6B | 480 mb; 0.5 mm; 30% st; 75 mgsf; | 16.2 | — |
| | 200 mgcxb; oven; 27.3% sf; 72.7% cxb | | |
| 177-7A | 120 mb; 0.5 mm; 20% st; 50 mgsf; | 23.7 | 33.8 |
| | 200 mgcxb; lyo; 20% sf; 80% cxb | | |
| 177-7B | 120 mb; 0.5 mm; 20% st; 50 mgsf; | 15.7 | — |
| | 200 mgcxb; oven; 20% sf; 80% cxb | | |
| 177-8A | 120 mb; 0.5 mm; 30% st; 75 mgsf; | 14.5 | −8.9 |
| | 200 mgcxb; lyo; 27.3% sf; 72.7% cxb | | |
| 177-8B | 120 mb; 0.5 mm; 30% st; 75 mgsf; | 15.8 | — |
| | 200 mgcxb; oven; 27.3% sf; 72.7% cxb | | |
| 177-9A | 120 mb; 0.5 mm; 40% st; 100 mgsf; | 14.1 | 5.2 |
| | 200 mgcxb; lyo; 33.3% sf; 66.7% cxb | | |
| 177-9B | 120 mb; 0.5 mm; 40% st; 100 mgsf; | 13.4 | — |
| | 200 mgcxb; oven; 33.3% sf; 66.7% cxb | | |
| 177-10 | CXB suspension | 106.2 | N/A |

Samples 177-6 (A and B, both oven dried and lyophilized), were prepared in manner identical to that of samples 177-8 (A and B, both oven dried and lyophilized), except for the boiling time of the silk fibroin. As previously sated, an increase in the boiling time reduces the molecular weight of the silk fibroin. Consequently, these samples allowed for the direct comparison of rods prepared identically with different molecular weights of silk fibroin. The lyophilized samples with a higher molecular weight (120 mb) exhibited an initial burst that was 21.1% less than the lyophilized samples prepared with a lower molecular weight (480 mb). Meanwhile, the oven dried samples with a higher molecular weight (120 mb) exhibited an initial burst that was 2.50% less than the oven dried samples prepared with a lower molecular weight (480 mb).

The daily release percentages were also compared to the initial burst percentages. The daily release percentages, as well as the ratio of the initial burst percentages to the daily release percentages, were calculated from the data from the in vitro release experiments, and these data were displayed in Table 25. The daily release percentages were calculated for the first 49 days of the study.

TABLE 25
Daily percentage of celecoxib release for rods of different
drying methods and different boiling times

| | | | | Initial | | |
|---|---|---|---|---|---|---|
| | | | | burst: | Ratio | Ratio |
| | | Initial | Daily | Daily | CXB:SF | CXB:SF |
| Lot | Sample Name | Burst % | Release % | release | Theoretical | Actual |
| 177-1A | 480 mb; 0.5 mm; 40% st; 100 mgsf; | 13.6 | 9.1 | 6.3 | 1.0 | 0.6 |
| | 100 mgcxb; lyo; 50% sf; 50% cxb | | | | | |
| 177-1B | 480 mb; 0.5 mm; 40% st; 100 mgsf; | 10.1 | 1.9 | 5.2 | 1.0 | 0.6 |
| | 100 mgcxb; oven; 50% sf; 50% cxb | | | | | |
| 177-2A | 480 mb; 0.5 mm; 40% st; 100 mgsf; | 15.4 | 2.0 | 7.9 | 1.5 | 0.9 |
| | 150 mgcxb; lyo; 40% sf; 60% cxb | | | | | |
| 177-2B | 480 mb; 0.5 mm; 40% st; 100 mgsf; | 12.1 | 1.9 | 6.4 | 1.5 | 0.9 |
| | 150 mgcxb; oven; 40% sf; 60% cxb | | | | | |
| 177-4A | 480 mb; 0.5 mm; 40% st; 100 mgsf; | 13.0 | 2.0 | 6.4 | 2.5 | 1.2 |
| | 250 mgcxb; lyo; 28.6% sf; 71.4% cxb | | | | | |
| 177-4B | 480 mb; 0.5 mm; 40% st; 100 mgsf; | 14.2 | 2.1 | 6.9 | 2.5 | 1.2 |
| | 250 mgcxb; oven; 28.6% sf; | | | | | |
| | 71.4% cxb | | | | | |
| 177-6A | 480 mb; 0.5 mm; 30% st; 75 mgsf; | 18.4 | 1.8 | 10.3 | 2.7 | 1.2 |
| | 200 mgcxb; lyo; 27.3% sf; 72.7% cxb | | | | | |
| 177-6B | 480 mb; 0.5 mm; 30% st; 75 mgsf; | 16.2 | 1.9 | 8.5 | 2.7 | 1.2 |
| | 200 mgcxb; oven; 27.3% sf; | | | | | |
| | 72.7% cxb | | | | | |
| 177-7A | 120 mb; 0.5 mm; 20% st; 50 mgsf; | 23.7 | 3.7 | 6.4 | 4.0 | 1.3 |
| | 200 mgcxb; lyo; 20% sf; 80% cxb | | | | | |
| 177-7B | 120 mb; 0.5 mm; 20% st; 50 mgsf; | 15.7 | 1.9 | 8.2 | 4.0 | 1.5 |
| | 200 mgcxb; oven; 20% sf; 80% cxb | | | | | |
| 177-8A | 120 mb; 0.5 mm; 30% st; 75 mgsf; | 14.5 | 1.9 | 7.5 | 2.7 | 1.4 |
| | 200 mgcxb; lyo; 27.3% sf; 72.7% cxb | | | | | |
| 177-8B | 120 mb; 0.5 mm; 30% st; 75 mgsf; | 15.8 | 1.9 | 8.2 | 2.7 | 1.4 |
| | 200 mgcxb; oven; 27.3% sf; | | | | | |
| | 72.7% cxb | | | | | |
| 177-9A | 120 mb; 0.5 mm; 40% st; 100 mgsf; | 14.1 | 1.9 | 7.5 | 2.0 | 1.1 |
| | 200 mgcxb; lyo; 33.3% sf; 66.7% cxb | | | | | |
| 177-9B | 120 mb; 0.5 mm; 40% st; 100 mgsf; | 13.4 | 1.8 | 7.4 | 2.0 | 1.0 |
| | 200 mgcxb; oven; 33.3% sf; | | | | | |
| | 66.7% cxb | | | | | |
| 177-10 | CXB Suspension | 106.2 | N/A | N/A | N/A | N/A |

Oven dried rods showed slower release than the lyophilized rods, with lower initial burst percentages, however they also showed similar biphasic release profiles. The second phase of release, however, was delayed from 10 to 14 days when the rods were oven dried. The complete release of CXB ranged from 35 to greater than 63 days and followed the same trends as the lyophilized rods (rates increasing with increasing CXB:Silk ratio). This slower release of the oven-dried rods was most likely due to increased beta-sheet content of the silk-fibroin as well as decreased porosity of the rods. Both factors would make the rods more hydrophobic, slowing water uptake and decreasing diffusion of CXB.

The data also revealed that the (w/w) ratio of API to silk fibroin was directly proportional to the initial burst percentage. In the context of the 0.5 mm silk fibroin rods, lower initial burst percentages corresponded with lower ratios of CXB to silk fibroin, while higher initial burst percentages corresponded to higher ratios of CXB to silk fibroin. The daily release percentage of the 0.5 mm rods also increased as the ratio of CXB to silk fibroin increased. As the drug loading increased and silk-fibroin concentration decreased, the release rates increased. This suggested that the silk-fibroin was controlling release and that release rates could be tuned using this variable.

The measured and calculated parameters of the rods were also examined in the context of silk fibroin boiling time and molecular weight, by comparing the experimental results from rods of lot numbers 177-6 (A and B) and 177-8 (A and B). As stated previously, the rods from these preparations were identical except for the boiling time, and therefore the molecular weight, of the silk fibroin. The ratio of the initial burst percentages to the daily release percentages was lower for rods prepared from higher molecular weight silk fibroin; this result was likely due to the observed lower initial burst percentage with silk rods of higher molecular weight silk fibroin. Meanwhile, the daily release percentages differed by only 0.1% between the freeze-dried rods with lower and higher molecular weights; the daily release percentages of these samples were 1.8% and 1.9% respectively. The daily release percentages did not change between oven dried samples of lower and higher molecular weight; the daily release percentage for those samples was 1.9%. As a result, it was concluded that the boiling time, and consequently the molecular weight, of the silk fibroin did not affect the daily release percentages of the silk fibroin rods. These in vitro characterizations displayed that release from these formulations was independent of the silk-fibroin molecular weights assessed.

### Example 11. In Vivo Study of Silk Fibroin Rods with Celecoxib in an Animal Model

As with the hydrogels without celecoxib (CXB), all buffers and stock solutions were prepared under sterile conditions unless otherwise indicated. All formulations were prepared with SOHO silk yarn. The poloxamer-188 was from Sigma-Aldrich (St. Louis, Mo.), while the PEG4 kDa was from Clariant, Charlotte N.C. Multiple preparations of the same formulations may be used in the study and overall analysis.

### Preparation of Celecoxib Experimental Controls

As seen in the hydrogels formulated with CXB, a 27.8% suspension of celecoxib (CXB) was prepared from 4.15 g dry heat treated (DHT) CXB (from Cipla, Miami Fla.) in 10.78 mL of 0.79% Polysorbate-80 (from Croda, Snaith UK) and mixed until homogenous. To prepare the 10% CXB suspension as a control, a 1.789 mL fraction of the 27.8% CXB suspension was diluted to 5 mL via the addition of 0.349 mL 315 mM PB (pH=7.4), 0.158 mL of 200 mg/mL NaCl, and DI water. The resulting 10% CXB suspension was immediately aliquoted into 0.2 mL fractions in 1 cc syringes so that it remained homogenous, and the fractions were stored on ice until subsequent injection. To prepare the 0.2% CXB suspension as an additional control, a 0.18 mL fraction of the 10% CXB solution was diluted with 0.686 mL of 315 mM PB (pH=7.4), 0.31 mL of 200 mg/mL NaCl, 2.468 mL of 0.79% Polysorbate-80, and DI water to a final volume of 10 mL. The suspension was mixed until homogenous, aliquoted into 0.2 mL fractions, and stored on ice until use.

### Preparation of Silk Fibroin Materials for Injection

The efficacy of the silk rods was compared to that of silk fibroin hydrogels. Both unadulterated silk fibroin hydrogels and silk fibroin hydrogels with 10% CXB were prepared as experimental controls. All processes were performed under aseptic conditions using pre-sterilized materials. To prepare the unadulterated silk fibroin hydrogel (sample 3B) 300 mg of 480 mb silk fibroin were brought up in 3.342 mL 0.6% Polysorbate-80, 0.383 mL of 315 mM phosphate buffer (pH=7.4), and 0.246 mL DI water. To prepare the 10% CXB hydrogel (sample 4B), 300 mg of 480 mb silk fibroin were brought up in 3.589 mL of the 27.8% CXB suspension and 0.381 mL of 315 mM PB (pH=7.4). Both the solutions for the hydrogel samples were incubated at room temperature and mixed for 30 minutes until homogenous. Each mixture was then aliquoted into 3.41 mL fractions in 10 cc syringes. The samples in Table 26 are named by the process used to prepare and formulate each hydrogel. For example, in the sample named 480 mb; hyd; 27.8% cxbst; 3% SFf; 10% CXBf; 10% P188f, “480 mb” refers to silk degummed with a 480-minute boil, “hyd” refers to the formulation of the sample as a hydrogel, “27.8% cxbst” refers to a preparation from a stock solution of 27.8% of celecoxib, “3% SFf” refers to a formulation with 3% (w/v) silk fibroin, “10% CXBf” refers to a formulation with 10% (w/v) celecoxib, and “10% P188f” refers to a formulation with 10% (w/v) poloxamer 188. The sample named “480 mb; 0.5 mm; 40% st; 100mgsf; 200mgcxb; lyo; 33.3% sf; 66.7% cxb” refers to a silk fibroin rod prepared from silk degummed with a 480-minute boil, an extrusion with a 0.5 mm diameter, a preparation from a 40% stock solution of silk fibroin, a preparation from 100 mg of silk fibroin, a preparation from 200 mg of celecoxib, lyophilization, a theoretical w/w percentage of 33.3% silk fibroin, and a theoretical w/w percentage of 66.7% celecoxib. All suspension and gel formulations contained 0.2% polysorbate-80 and 22 mM phosphate buffer. The 1.4% CXB suspension contained 6.34 mg/mL NaCl. The 10% CXB suspension contained 6.32 mg/mL NaCl. Both hydrogels contained 5.94 mg/mL NaCl. The rods contained 74.1 mM phosphate buffer.

TABLE 26
Descriptions of samples for in vivo experiments of silk fibroin rods with celecoxib

| | | Silk- | Silk- | | |
|---|---|---|---|---|---|
| | | Fibroin | Fibroin | | Excipient |
| Sample Name | Description | Boil Time | Conc. (%) | Excipient | Conc. (%) |
| 1.4% CXB control | 1.4% CXB Suspension | — | — | — | — |
| 10% CXB control | 10% CXB Suspension | — | — | — | — |
| 480 mb; hyd; 0% cxbst; | 3% 480 mb; 10% P188 | 480 | 3 | P188 | 10 |
| 3% SFf; 0% CXBf; | | | | | |
| 10% P188f | | | | | |
| 480 mb; hyd; 27.8% cxbst; | 3% 480 mb; 10% P188; | 480 | 3 | P188 | 10 |
| 3% SFf: 10% CXBf; | 10% CXB | | | | |
| 10% P188f | | | | | |
| 480 mb; 0.5 mm; 40% st; | 20% 480 mb; 40% CXB | 480 | 20 | — | — |
| 100 mgsf; 200 mgcxb; lyo; | Rods | | | | |
| 33.3% sf; 66.7% cxb | | | | | |

An excipient solution was prepared from 13.05 mL of stock 20% P188, 0.777 mL of 200 mg/mL NaCl, and 1.173 mL of DI water. This excipient solution was prepared in 10 cc syringes in 4.59 mL aliquots. For each sample, the syringe of the representative silk fibroin solution was connected to a syringe of its designated excipient solution via a B Braun fluid dispensing connector. The contents of the syringes were then mixed until homogenous. The resulting samples were incubated on a rotator for 24 hours at 37° C. and then separated into 0.2 mL aliquots in 1 cc syringes. The pH values of the samples were measured with a glass pH probe. Samples were stored at 4° C., as needed. Formulations of the hydrogels contained 1.04% (w/v) sodium chloride, 0.2% (w/v) Polysorbate-80, and 22 mM phosphate buffer at pH=7.4 for the P188-containing hydrogels. Some formulations comprised 10% P188, 10% CXB, and 10.4 mg/mL sodium chloride at a pH of 7.4.

The silk fibroin rods were prepared as described in the preparation of 0.5 mm silk fibroin rods. Briefly, 600 mg of 480 mb silk fibroin were dissolved in 0.900 mL of DI water. 0.591 mL of the resulting solution was then used to bring up 473 mg of CXB, vortexed, and mixed. The mixture of silk fibroin and CXB was further mixed back and forth through a syringe connector until the mixture was homogenous. The mixture was then capped with a 27-gauge, 0.5-inch, needle and extruded into 10 cm lengths of 0.02″ ID PEEK tubing. The tubing was cut into 2 cm pieces and incubated overnight at 37° C. under sterile conditions. The rods were then removed from the tubing, frozen, and lyophilized overnight. Lyophilized rods were stored at 4° C. until injection. The rod-containing sample is named by the process used to prepare and formulate each silk rod. For example, the sample named “480 mb; 0.5 mm; 40% st; 100mgsf; 200mgcxb; lyo; 33.3% sf; 66.7% cxb” refers to a silk fibroin rod prepared from silk degummed with a 480-minute boil, an extrusion with a 0.5 mm diameter, a preparation from a 40% stock solution of silk fibroin, a preparation from 100 mg of silk fibroin, a preparation from 200 mg of celecoxib, lyophilization, a theoretical w/w percentage of 33.3% silk fibroin, and a theoretical w/w percentage of 66.7% silk fibroin. CXB loaded rods were cut to 1 cm lengths and preloaded into 21G, 1″ needles. The final formulations of the rods also contained trace amounts of potassium phosphate buffer (phosphate buffer monobasic and phosphate buffer dibasic).

### In Vitro Release Profile of Hydrogel for In Vivo Experiments

The silk fibroin hydrogels were subject to the in vitro release experiments used to analyze silk hydrogels of varying concentration and silk fibroin boiling time. Briefly, In triplicate, 50 mg of each formulation were weighed into half of a #4 gelatin capsule (MyHerbar, Dallas Tex.). Capsules were added to 45 mL of release medium (1× phosphate buffered saline, 2% polysorbate-80, and 0.05% sodium azide). It had previously been shown that the solubility of celecoxib in this release media is 850 μg/mL. 45 mL of this release media allowed for 38 mg CXB solubility. This media will ensure sink conditions (greater than or equal to 5 times the CXB solubility) are maintained throughout the study. The tubes were incubated at 37° C. with shaking. 1 mL of the release media was collected from each sample at days 1, 4, 7, 10, 14 and weekly thereafter and replaced with fresh media. At each timepoint, the tubes were placed upright for at least 15 minutes to allow the formulation to settle prior to taking the sample. Release media was analyzed by HPLC (Agilent 1290 HPLC system) at 260 nm

Controls were prepared at Day 0 by weighing 50 mg of each formulation in triplicate in separate glass vials. Methanol was added to each sample to extract CXB. Samples were placed on a shaker at room temperature for 24 hrs. The supernatant was analyzed by HPLC to determine CXB loading.

The plot of the cumulative percentage of API released over time can be seen in FIG. 4. The release of the API from the hydrogel was much slower than the CXB suspension, which served as a control. The release of API from the hydrogel followed first order kinetics, and it occurred over the span of 1 month. The initial burst was approximately 40%.

### In Vitro Release Profile of Rods for In Vivo Experiments

The silk fibroin rods were subject to the in vitro release experiments used to analyze silk fibroin rods of both 1 mm and 0.5 mm diameter loaded with CXB. Briefly, 1 cm segments of rod were weighed into 50 mL conical tubes. 45 mL of release medium (phosphate buffered saline, 0.3% polysorbate-80, and 0.05% sodium azide) was added to each tube. It had previously been shown that this media would ensure sink conditions (greater than or equal to 5 times CXB solubility) were maintained throughout the study. The tubes were incubated at 37° C. with shaking. 1 mL of the release media was collected from each sample at days 1, 4, 7, 10, 14 and weekly thereafter and replaced with fresh media.

Controls were prepared by weighing 1 cm of each formulation in triplicate in separate glass vials. Methanol was added to each vial. Samples were vortexed, sonicated, and placed on a shaker at RT for 24 hrs. The supernatant was analyzed by HPLC to determine CXB loading (mg/g).

Release media was analyzed for CXB concentration by HPLC-UV (Agilent 1290 HPLC system) 260 nm. The average cumulative percentage of API released over time was listed in Table 27 and FIG. 4. The release of CXB followed near zero-order kinetics. Cumulative percent released was calculated with a daily standard curve unless otherwise indicated. In Table 27, “Std. Dev.” refers to standard deviation.

TABLE 27
In vitro release kinetics of celecoxib from 480 mb; 0.5
mm; 40% st; 100 mgsf; 200 mgcxb; lyo; 33.3% sf; 66.7% cxb

| | | | Average Cumulative | Std. Dev. |
|---|---|---|---|---|
| | Average | | % Released | (Calculated |
| | Cumulative % | Std. | (Calculated with | with single |
| Day | Released | Dev. | single standard) | standard) |
| 0 | 0.0 | 0 | 0.0 | 0 |
| 1 | 12.5 | 1.2 | 11.1 | 1.0 |
| 3 | 26.4 | 2.0 | 23.4 | 1.4 |
| 7 | 50.4 | 3.0 | 41.0 | 2.4 |
| 10 | 58.0 | 4.2 | 50.1 | 3.5 |
| 14 | 69.5 | 5.0 | 60.8 | 4.4 |
| 21 | 79.0 | 5.7 | 74.0 | 5.1 |
| 28 | 90.4 | 5.5 | 85.4 | 5.4 |
| 35 | 105.2 | 5.7 | 94.4 | 5.2 |
| 42 | 112.1 | 5.3 | 101.2 | 4.3 |
| 49 | 119.7 | 4.7 | 103.5 | 4.0 |
| 56 | — | — | 102.9 | 3.4 |
| 63 | — | — | 102.3 | 3.2 |

Additional parameters of the silk fibroin rods were also explored in Table 28. First the actual loading of CXB was determined by UPLC to be 48.4%, which was slightly higher than the theoretical loading percentage. The initial burst percentage, 11.1° %, was then analyzed in comparison with the daily release percentage, 1.6%. The ratio of the initial burst percentage to the daily release percentage was determined to be 7.1. Overall, the rods were demonstrated to be capable of releasing the API, CXB, over a period of 49 days, and this gradual release rendered these rods acceptable candidates for in vivo studies. In Table 28, “Std. Dev.” refers to standard deviation.

TABLE 28
Examining the loading and kinetics of 480 mb; 0.5 mm;
40% st; 100 mgsf; 200 mgcxb; lyo; 33.3% sf; 66.7% cxb

| Sample No. | 179 |
|---|---|
| Sample Name | 480 mb; 0.5 mm; 40% st; 100 mgsf; |
| | 200 mgcxb; lyo; 33.3% sf; 66.7% cxb |
| Theoretical % CXB (w/w) | 66.7 |
| Actual % CXB (w/w) | 48.4 |
| Std. Dev. of actual CXB % (w/w) | 0.6 |
| Initial Burst % | 12.5 |
| Initial Burst % (single standard) | 11.1 |
| Daily release % | 2.2 |
| Daily release % (single standard) | 1.6 |
| Initial Burst:Daily release | 5.6 |
| Initial Burst:Daily release | 7.1 |
| (single standard) | |

Administration of 0.5 mm Rods with Celecoxib

New Zealand adult white rabbits were prepared and draped in the usual sterile fashion. Intravitreal injections were made into the left eye (OS) of all rabbits. Right eyes remained as naïve controls. Animals were given a pre-anesthetic (Xylazine 1.1 mg/kg IM, Buprenorphine HCl 2-6 mcg/kg IM). Animals were then anesthetized with ketamine 22 mg/kg IM. The animals were placed on a heating pad and their vitals were monitored. The animals were put on inhalation anesthesia (Isoflurane at 1.5-2%) with 02 supplement.

To administer the hydrogels into the intravitreal space, a lid speculum was inserted into the rabbit's left eye. The conjunctiva was rinsed with BSS solution. Then, the conjunctival sac was prepped with a 5% ophthalmic betadine solution. The hydrogel was then injected into the intravitreal space using a double-plane injection technique. The sclera was penetrated at 15°-30°, then the needle was repositioned to a 45°-60° angle while the sclera was still engaged; the formulation was delivered and the needle was removed at a 90 angle. Following injection, the central retinal artery was examined via indirect ophthalmoscopy to confirm perfusion and 1-2 drops of betadine solution were added to the conjunctiva prior to removal of the speculum.

To administer the rods, formulations were pre-loaded into sterile 21 g, 1″ needle cannulas. Intracannular plungers were fashioned with 28G wire which were pre-cut, sterilized, and placed into the needle from the hub. The same sterile and double-plane injection technique was used as for the hydrogels. The sclera was penetrated at 15°-30°, then the needle was repositioned to a 45°-60° angle while the sclera was still engaged; the formulation was delivered. The plunger was depressed, resulting in complete delivery of the rod into the eye into the intravitreal space. The wire could be pushed until it extended beyond the needle or cannula to ensure complete delivery. The needle was removed at a 90° angle. Following injection, the central retinal artery was examined via indirect ophthalmoscopy to confirm perfusion and 1-2 drops of betadine solution were added to the conjunctiva prior to removal of the speculum. When fully injected, the rod was clear from the wall of the eye.

Intraocular Pressure and Biocompatibility after Rod Administration

Intraocular pressure was measured with a Tono-Pen. 7 days after rod administration, there were no obvious signs of inflammation. No elevation in intraocular pressure was detected as compared to the naïve contralateral eyes. There were slightly lower intraocular pressures detected in the eyes treated with celecoxib, as seen in Table 29. As a result, the celecoxib loaded rods reduced the intraocular pressure of the treated eye. The analysis of the intraocular pressure was continued over the course of the study, as seen in Table 29, and multiple preparations of the same rod formulations were used. The intraocular pressure in the eyes containing the silk fibroin rod did not increase over the time evaluated.

No adverse clinical findings were noted throughout the course of the study. Mild-vitreous hemorrhage was sometimes observed following rod injection. Similar findings were seen previously with silk-fibroin solutions and hydrogels with CXB. Additionally, the histopathology report indicated that the rods did not induce any inflammation in the vitreous. There was slight infiltration of macrophages into the silk-fibroin rods, but there were no signs of inflammation or damage in the remainder of the eye. In addition, normal or lower intraocular pressure was measured 4 months after rod administration. No local inflammation, hemorrhage, or other complications were detected 4 months after administration. Based on these results, intravitreal injections of silk-fibroin rods were determined to be well tolerated in rabbits.

TABLE 29
Intraocular pressure measurements at exams with silk fibroin rods (480 mb;
0.5 mm; 40% st; 100 mgsf; 200 mgcxb; lyo; 33.3% sf; 66.7% cxb)

| | | Left Eye (Injected) | | Right Eye (Naïve) | | |
|---|---|---|---|---|---|---|
| | | | | Average | | Fold change in |
| | | Average IOP | Standard | IOP | Standard | IOP |
| Group | Day | (mmHg) | Deviation | (mmHg) | Deviation | (Injected/Naïve) |
| 1 | 47 | 9.00 | 2.00 | 12.00 | 0.00 | 0.75 |
| | 30 | 12.33 | 4.04 | 11.67 | 5.86 | 1.06 |
| | 88 | 10.75 | 3.10 | 11.75 | 1.50 | 0.91 |
| | 111 | 12.33 | 4.04 | 11.00 | 5.20 | 1.12 |
| | 126 | 8.00 | 4.08 | 10.00 | 4.90 | 0.80 |
| | 169 | 10.00 | 1.41 | 10.50 | 2.12 | 0.95 |
| Control | 30 | 5.33 | 3.51 | 11.33 | 1.15 | 0.47 |
| (1.4% | | | | | | |
| CXB | | | | | | |
| solution | | | | | | |

### Example 12. In Vivo Study of Silk Fibroin Hydrogels in an Animal Model

All buffers and stock solutions were prepared under sterile conditions unless otherwise indicated. All formulations were prepared with silk yarn purchased from SOHO. The silk rods were prepared with a dose of 750 μg of celecoxib (CXB) (from Cipla, Miami Fla.). The poloxamer-188, sodium chloride, and hydrochloric acid were from Sigma-Aldrich (St. Louis, Mo.), while the PEG4 kDa was from Clariant, Charlotte N.C. Polysorbate-80 was purchased from Croda (Snaith UK). Potassium phosphate monobasic and potassium phosphate dibasic were purchased from Sigma Aldrich Fine Chemical (SAFC, St. Louis Mo.). Phosphate buffered saline was purchased from Gibco (USA). Multiple preparations of the same formulations were used.

### Preparation of Silk Fibroin Experimental Controls

A phosphate buffer (PB) control was prepared for the in vivo experiments. PB was aliquoted into 0.4 mL fractions and stored in 1 cc syringes. The PBS controls were stored at 4° C. until time of injection.

A CXB suspension was also prepared as an experimental control. CXB was suspended in an aqueous solution of sodium chloride (Sigma-Aldrich, St. Louis, Mo.), Polysorbate-80 (Croda, Snaith UK), and phosphate buffer. The CXB was homogeneously dispersed using ultrasonication and stored at 4° C. prior to injection. The suspension drawn up into 1 cc. syringes just prior to injection to avoid settling.

Silk fibroin solutions were prepared by boiling raw silk (from Jiangsu SOHO) for 120 minutes (herein referred to as “120 mb”) or by boiling for 480 minutes (herein referred to as “480 mb”). 120-minute boil results in silk fibroin with a higher molecular weight than the 480-minute boil. Lyophilized silk-fibroin was reconstituted with an aqueous solution of sodium chloride, Polysorbate-80, and phosphate buffer. The fibroin was allowed to fully reconstitute prior to being drawn into a 6-cc. syringe. Sodium chloride concentration was adjusted to ensure a final osmolarity of 280 mOsm. During preparations, 300 mg of 120 mb silk fibroin was brought up in 3.33 mL of 0.6% Polysorbate-80, 0.317 mL of 200 mg/mL NaCl, and 5.672 mL of DI water. Concurrently, 300 mg of 480 mb silk fibroin was brought up in 3.33 mL 0.6% Tween-80, 0.381 mL of 200 mg/mL NaCl, and 5.675 mL DI water. Each individual solution was mixed and incubated at room temperature for 30 minutes to dissolve the silk fibroin. The resulting solutions were stored at 4° C. and aliquoted into 1 cc. syringes prior to injection.

### Preparation of Hydrogels

The hydrogel samples were prepared as described below. The lyophilized silk fibroin was allowed to fully reconstitute prior to being drawn into a 6-cc. syringe. During preparation, 300 mg of 120 mb silk fibroin or 480 mb silk fibroin were brought up in 3.342 mL 0.6% Polysorbate-80, 0.383 mL of 315 mM PB (pH=7.4), and 0.246 mL DI water. Each solution was mixed and incubated at room temperature for 30 minutes to dissolve the silk fibroin. The mixtures were aliquoted into 2.13 mL fractions in 3, 6 cc, syringes. The samples in Table 30 were named by the process used to prepare and formulate each hydrogel. For example, in the sample named 120 mb; hyd; 0% cxbst; 3% SFf; 0% CXBf; 40% PEG4kf, “120 mb” refers to silk degummed with a 120-minute boil, “hyd” refers to the formulation of the sample as a hydrogel, “0% cxbst” refers to a preparation from a stock solution of 0% of celecoxib, “3% SFf” refers to a formulation with 3% (w/v) silk fibroin, “0% CXBf” refers to a formulation with 0% (w/v) celecoxib, and “40% PEG4kf” refers to a formulation with 40% (w/v) PEG4k. Some samples were prepared with P188 (% P188f). Some samples were prepared with silk fibroin degummed with a 120-minute boil (120 mb). The 120 mb solution control contained 0.2% Polysorbate-80, 22 mM phosphate buffer, and 6.34 mg/mL NaCl. The 480 mb solution control contained 0.2% Polysorbate-80, 22 mM phosphate buffer, and 6.28 mg/mL NaCl. The 120 mb hydrogel with PEG4k contained 0.2% Polysorbate-80, 22 mM phosphate buffer, 2.97 mg/mL NaCl, and 15 mM HCl. The 120 mb hydrogel with P188 contained 0.2% Tween-80, 22 mM phosphate buffer, and 5.99 mg/mL NaCl. The 480 mb hydrogel with P188 contained 0.2% Polysorbate-80, 22 mM phosphate buffer, and 5.95 mg/mL NaCl.

TABLE 30
Descriptions of samples for in vivo silk fibroin hydrogel experiments

| | | Silk- | Silk- | | | |
|---|---|---|---|---|---|---|
| | | Fibroin | Fibroin | | | Ratio SF |
| | | Boil | Conc. | | Excipient | to |
| Sample name | Description | Time | (%) | Excipient | Conc. (%) | Excipient |
| PBS control | PBS | — | — | — | — | — |
| 120 mb control | 120 mb | 120 | 3 | — | — | — |
| | Solution | | | | | |
| 480 mb control | 480 mb | 480 | 3 | — | — | — |
| | Solution | | | | | |
| 120 mb; hyd; 0% cxbst; 3% SFf; | 3% 120 mb; | 120 | 3 | PEG | 40 | 0.075 |
| 0% CXBf; 40% PEG4kf | 40% PEG | | | 4 kDa | | |
| | 4 kDa | | | | | |
| 120 mb; hyd; 0% cxbst; 3% SFf; | 3% 120 mb; | 120 | 3 | P188 | 10 | 0.3 |
| 0% CXBf; 10% P188f | 10% P188 | | | | | |
| 480 mb; hyd; 0% cxbst; 3% SFf; | 3% 480 mb; | 480 | 3 | P188 | 10 | 0.3 |
| 0% CXBf; 10% P188f | 10% P188 | | | | | |

Excipient solutions were prepared so that a 0.75:1 mix of silk-fibroin solution: excipient solution would result in the desired final formulations. The pH of polyethylene glycol (PEG) hydrogels was adjusted using hydrochloric acid (from Sigma, St. Louis, Mo.) to account for the changes in pH observed when mixing phosphate buffer and PEG. The excipient solutions were drawn up into a second 6 mL. syringe. The corresponding solutions of excipients were prepared as described in Table 31. A 2.87 mL volume of each excipient was aliquoted into a syringe for subsequent mixing with the silk fibroin to generate the desired formulation. Excipients included NaCl, polyethylene glycol (PEG), and poloxamer 188 (P188). For each sample, the syringe of the representative silk fibroin solution was connected to a syringe of its designated excipient solution via a B Braun fluid dispensing connector. The contents of the syringes were then mixed until homogenous. The hydrogels had an osmolarity of 280 mOsm. The resulting samples were incubated on a rotator for 24 hours at 37° C. The pH values of the samples were measured with a glass pH probe, and they were adjusted with hydrochloric acid. The samples had a final (w/v) ratio of silk fibroin to excipient of between 0.01 and 0.5. The samples were then separated into 0.4 mL aliquots in 1 cc syringes, and they were stored at 4° C. until time of injection. Formulations of the hydrogels contained sodium chloride, 0.2% (w/v) Polysorbate-80, and 22 mM phosphate buffer at pH=7.4 for the P188-containing hydrogels. Some hydrogel formulations comprised 10% P188 and 10.4 mg/mL sodium chloride with a pH of 7.4. Formulations contained hydrochloric acid, sodium chloride, 0.2% (w/v) Polysorbate-80, and 22 mM phosphate buffer at pH=7.4 for the PEG 4 kDa-containing hydrogels. Some formulations contained 401% PEG 4 kDa, 5.2 mg/mL sodium chloride, and 22 mM hydrochloric acid, with a pH of 7.4.

TABLE 31
Solution preparations for excipients

| | NaCl | | | | | | |
|---|---|---|---|---|---|---|---|
| | mg/mL | mL | mL | mL | uL 200 | | uL |
| | needed in | to | stock | stock | mg/mL | uL DI | 1N |
| Sample | exc. | make | PEG | P188 | NaCl | Water | HCl |
| 120 mb; hyd; 0% cxbst; 3% SFf; | 5.17 | 4 | 3.72 | 0 | 103 | 69 | 108 |
| 0% CXBf; 40% PEG4kf | | | | | | | |
| 120 mb; hyd.; 0% cxbst; 3% SFf; | 10.43 | 4 | 0 | 3.48 | 208.6 | 311 | 0 |
| 0% CXBf; 10% P188f | | | | | | | |
| 480 mb; hyd; 0% cxbst; 3% SFf; | 10.36 | 4 | 0 | 3.48 | 207.2 | 313 | 0 |
| 0% CXBf; 10% P188f | | | | | | | |

### Administration of Hydrogels

The subjects were New Zealand white rabbits with a mass of 3-4 kg. The rabbits were separated into six groups, with three rabbits in each group. Each group was given an intravitreal injection with the formulation as described in Table 32. All injections were performed in the left eye, with the right eye remaining naïve to serve as an intra-animal control.

TABLE 32
Experimental groups of rabbits for
the study of silk fibroin hydrogels

| | | Name of Samples |
|---|---|---|
| Group | Description | Administered |
| 1 | PBS | PBS control |
| 2 | 120 mb Solution | 120 mb control |
| 3 | 480 mb Solution | 480 mb control |
| 4 | 3% 120 mb; 40% PEG 4 kDa | 120 mb; hyd; 0% cxbst; 3% SFf; |
| | | 0% CXBf; 40% PEG4kf |
| 5 | 3% 120 mb; 10% P188 | 120 mb; hyd; 0% cxbst; 3% SFf; |
| | | 0% CXBf; 10% P188f |
| 6 | 3% 480 mb; 10% P188 | 480 mb; hyd; 0% cxbst; 3% SFf; |
| | | 0% CXBf; 10% P188f |

All silk fibroin hydrogel formulations were pre-loaded into sterile 1 cc syringes, with 0.4 mL in each syringe. Prior to injection, the syringe cap was removed, and a sterile 27-gauge, ½″ needle was attached. The volume was adjusted to 0.1 mL, and the formulation was injected into the intravitreal space, 2 mm posterior to the limbus.

All procedures were performed under general anesthesia. Animals were given a pre-anesthetic (Xylazine 1.1 mg/kg IM, Buprenorphine HCl 2-6 mcg/kg IM). Animals were then anesthetized with ketamine 22 mg/kg IM. Animals were placed on a heating pad, and vitals were monitored. Animals were put on inhalation anesthesia (Isoflurane at 1.5-2%) with 02 supplement.

All rabbits had their peri-ocular fur of the left eye trimmed prior to the procedure. A wire lid speculum was used to hold the eye open. The eye was rinsed with balanced salt solution (BSS), followed by a rinse with 5% ophthalmic betadine. The betadine was applied again, immediately prior to the injection and post-injection. All rabbits received gentamycin ophthalmic ointment to the operative (left) eye in the recovery area post-procedure.

To administer the hydrogels into the intravitreal space, a lid speculum was inserted into the rabbit's left eye. The conjunctiva was rinsed with BSS solution. Then, the conjunctival sac was prepped with a 5% ophthalmic betadine solution. The hydrogel was then injected into the intravitreal space using the double panel technique described in the earlier in vivo studies of rods and gels. The formulation was delivered to the intravitreal space, and the needle was removed. Following injection, the central retinal artery was examined via indirect ophthalmoscopy to confirm perfusion and 1-2 drops of betadine solution were added to the conjunctiva prior to removal of the speculum.

Intraocular Pressure and Biocompatibility after Hydrogel Injection

Immediately following the injection, it was noted that the smaller size of the animals used in the study lead to hypoperfusion upon injection of 0.1 mL of material. The rabbits had a mass of approximately 3 kg. The hydrogels injected into animals from groups 4-6 formed well defined, cohesive, spherical depots upon injection. These opaque formulations were easily visualized. The hydrogels for the experiments on the rabbits in group 4 (120 mb; hyd; 0% cxbst; 3% SFf; 0% CXBf; 40% PEG4kf) were too difficult to inject. The injection of this formulation was concluded to not be feasible without the use of an auto-injector. In addition, the low molecular weight silk hydrogels, used in the formulations for group 6, were less opaque than the formulations with high molecular weight silk, used on groups 4 and 5.

48 hours after injection, 8 days after injection, and 9 days after injection the intraocular pressure was measured with a Tono-Pen (see Table 33 for results). Anterior penlight exams and posterior dilated fundus exams were also performed at these times. 48 hours after the injection, all animals exhibited slight conjunctival irritation. This result was attributed to the betadine solution used during the procedure. All silk hydrogel formulations, as seen in groups 4-6, were unchanged. The depots were located at the base of the eye, out of the visual field, and they were cohesive and opaque. The depots from the formulations used in group 6 (480 mb; hyd; 0%/cxbst; 3% SFf; 0% CXBf; 10% P188f) were less opaque than those of the other hydrogels. The standard deviation of the intraocular pressure of the right eye for subjects in group 4 (noted by “*”) was not calculable because only one animal had the IOP of the right eye measured with a method identical to the rest, rendering n=1 for direct comparisons.

TABLE 33
Intraocular pressure measurements at 48-hour exam with silk fibroin hydrogels

| | | Left | | | | |
|---|---|---|---|---|---|---|
| | | Eye (Injected) | | Right Eye (Naïve) | | |
| | | Average | | Average | | Fold change in |
| | | IOP | Standard | IOP | Standard | IOP |
| Group | Sample Name | (mmHg) | Deviation | (mmHg) | Deviation | (Injected/Naïve) |
| 1 | PBS control | 11.33 | 0.58 | 11 | 2.65 | 1.03 |
| 2 | 120 mb control | 11.33 | 2.08 | 11.5 | 0.71 | 0.99 |
| 3 | 480 mb control | 10.33 | 1.53 | 12 | 1.41 | 0.86 |
| 4 | 120 mb; hyd; 0% cxbst; | 16.33 | 3.21 | 20 | 0.00* | 0.82 |
| | 3% SFf; 0% CXBf; | | | | | |
| | 40% PEG4kf | | | | | |
| 5 | 120 mb; hyd; 0% cxbst; | 14.67 | 2.52 | 16.33 | 4.04 | 0.90 |
| | 3% SFf; 0% CXBf; | | | | | |
| | 10% P188f | | | | | |
| 6 | 480 mb; hyd; 0% cxbst; | 15.5 | 2.12 | 17 | 2.65 | 0.91 |
| | 3% SFf; 0% CXBf; | | | | | |
| | 1.0% P188f | | | | | |

All silk solutions were determined to be well tolerated via the pen light exam at this timepoint. There were no signs of intraocular inflammation or irritation. Any slight hypoperfusion due to the volume of the injection had been resolved by this time. Compared to the naïve contralateral eyes (the right eyes), no elevation in intraocular pressure (IOP) was measured with the Tono-Pen. The fold change in IOP between the average IOP of injected (left) eye and the average IOP of naïve (right) eye for each group was also calculated. In most cases, the IOP of the left eye was measured to be slightly lower than that of the right eye (the control).

With the exception of the PBS control, i.e., group 1, in all instances the fold change in the IOP between the injected and the naïve eye in each group was less than one, which indicated that all formulations with silk reduced intraocular pressure. Group 4, where the silk was formulated with 40% PEG (4 kDa), showed the lowest fold change value, which indicated that this formulation was the most effective in reducing the intraocular pressure.

8 to 9 days after the injection, all conjunctival irritation had subsided. All silk hydrogel formulations (groups 4-6) were mainly unchanged since the 48-hour examination. The depots were still present at the base of the eye, out of the visual field, and they were still cohesive and opaque. The depots from the formulations used in group 6 (480 mb; hyd; 0% cxbst; 3% SFf; 0% CXBf; 10% P188f) were still less opaque than those of the other hydrogels. The intraocular pressure measurements using a Tono-Pen were also made at day 8/9 following the hydrogel injection. The results were shown in Table 34.

TABLE 34
Intraocular pressure measurements at 8 or 9-day exam
with silk fibroin hydrogels

| | | Left | | | | |
|---|---|---|---|---|---|---|
| | | Eye (Injected) | | Right Eye (Naïve) | | |
| | | Average | | Average | | |
| | | Left Eye | | Right Eye | | Fold change in |
| | | IOP | Standard | IOP | Standard | IOP |
| Group | Sample name | (mmHg) | Deviation | (mmHg) | Deviation | (Injected/Naïve) |
| 1 | PBS control | 8.00 | 4.00 | 13.00 | 3.61 | 0.62 |
| 2 | 120 mb control | 11.67 | 0.58 | 13.67 | 2.08 | 0.85 |
| 3 | 480 mb control | 13.00 | 2.65 | 14.33 | 2.52 | 0.91 |
| 4 | 120 mb; hyd; | 15.67 | 1.53 | 17.33 | 4.93 | 0.90 |
| | 0% cxbst; 3% SFf; | | | | | |
| | 0% CXBf; | | | | | |
| | 40% PEG4kf | | | | | |
| 5 | 120 mb; hyd; | 16.00 | 10.44 | 10.33 | 3.79 | 1.55 |
| | 0% cxbst; 3% SFf; | | | | | |
| | 0% CXBf; | | | | | |
| | 10% P188f | | | | | |
| 6 | 480 mb; hyd; | 12.00 | 3.61 | 24.33 | 10.41 | 0.49 |
| | 0% cxbst; 3% SFf; | | | | | |
| | 0% CXBf; | | | | | |
| | 10% P188f | | | | | |

All hydrogel formulations, silk solutions, and PBS solutions were determined to be well tolerated via clinical examination. There were no signs of intraocular inflammation or irritation. Compared to the naïve contralateral eyes (the right eyes), no elevation in intraocular pressure (IOP) was measured with the Tono-Pen. Animals in groups 1-4 were sacrificed 9 days post-injection. Animals in groups 4-6 were sacrificed 8 days post-injection

After 8-9 days, the fold change of the intraocular pressures between the injected eye and the naïve eye changed more drastically. Almost every group experienced a decrease in the fold change, which indicated that these formulations with silk reduced intraocular pressure more drastically over time. Group 6 (480 mb; hyd; 0% cxbst; 3% SFf; 0% CXBf; 10% P188f) showed the lowest fold change value, which indicated that this formulation was the most effective in reducing the intraocular pressure. Meanwhile, group 5 (120 mb; hyd; 0% cxbst; 3% SFf; 0% CXBf; 10% P188f), the hydrogels of which were formulated with a higher molecular weight silk fibroin, experienced an increase in the fold change, which indicated that this formulation increased intraocular pressure over time.

### Example 13. In Vivo Study of Silk Fibroin Hydrogels with Celecoxib in an Animal Model

As seen in the studies of silk fibroin hydrogels formulated without a therapeutic agent, all buffers and stock solutions were prepared under sterile conditions unless otherwise indicated. All formulations were prepared with SOHO silk yarn. The poloxamer-188, sodium chloride, and hydrochloric acid were from Sigma-Aldrich (St. Louis, Mo.), the PEG4 kDa was from Clariant, Charlotte N.C., and the celecoxib (CXB) was from Cipla, Miami Fla. Polysorbate-80 was purchased from Croda (Snaith UK). Potassium phosphate monobasic and potassium phosphate dibasic were purchased from Sigma Aldrich Fine Chemical (SAFC, St. Louis Mo.). Phosphate buffered saline was purchased from Gibco (USA). Multiple preparations of the same formulations were used.

### Preparation of Celecoxib Experimental Controls

All controls were prepared as described for the in vivo experiments of silk fibroin hydrogels with no therapeutic agent. Briefly, a 27.8% suspension of celecoxib (CXB) was prepared from 4.15 g dry heat treated (DHT) CXB in 10.78 mL of 0.79% Polysorbate-80 and mixed until homogenous. 1.789 mL of the 27.8% CXB suspension was diluted to 5 mL via the addition of 0.349 mL 315 mM PB (pH=7.4), 0.158 mL of 200 mg/mL NaCl, and DI water. The resulting 10% CXB suspension was immediately aliquoted into 0.4 mL fractions in 1 cc syringes so that it remained homogenous, and the fractions were stored on ice until injection.

Preparation of Silk Fibroin Hydrogels with 10% Celecoxib

The hydrogel samples were prepared as described in the experiments on hydrogels without a therapeutic agent. Hydrogels were prepared from both high molecular weight (120 mb) and low molecular weight (480 mb) silk fibroin. 300 mg of either 120 mb or 480 mb silk fibroin were brought up in 3.589 mL of the 27.8% CXB suspension and 0.381 mL of 315 mM PB (pH=7.4). The resulting solutions were incubated at room temperature and mixed for 30 minutes until homogenous. The silk fibroin solutions were then aliquoted into 2.13 mL fractions in 5 cc syringes. The samples in Table 35 are named by the process used to prepare and formulate each hydrogel. For example, in the sample named 120 mb; hyd; 27.8% cxbst; 3% SFf; 10% CXBf; 10% P188f, “120 mb” refers to silk degummed with a 120-minute boil, “hyd” refers to the formulation of the sample as a hydrogel, “27.8% cxbst” refers to a preparation from a stock solution of 27.8% of celecoxib, “3% SFf” refers to a formulation with 3% (w/v) silk fibroin, “10% CXBf” refers to a formulation with 10% (w/v) celecoxib, and “10% P188f” refers to a formulation with 10% (w/v) poloxamer 188. Some samples were prepared with silk fibroin degummed with a 120-minute boil (120 mb). The 10% CXB suspension contained 0.2% Tween-80, 22 mM phosphate buffer, and 6.32 mg/mL NaCl. The 120 mb hydrogel contained 0.2% Tween-80, 22 mM phosphate buffer, and 5.99 mg/mL NaCl. The 480 mb hydrogel contained 0.2% Tween-80, 22 mM phosphate buffer, and 5.95 mg/mL NaCl.

TABLE 35
Descriptions of samples for in vivo experiments with 3% (w/v) silk fibroin (SF)
hydrogels formulated with 10% (w/v) celecoxib and 10% P188

| | | | | | Ratio SF | | Ratio CNB | |
|---|---|---|---|---|---|---|---|---|
| | | SF | SF | P188 | to | Ratio | to | |
| Sample | | Boil | Conc. | Conc. | Excipient | CXB | Excipient | CNB:SF: |
| Name | Description | Time | (%) | (%) | (P188) | to SF | (P188) | P188 |
| 10% CXB | 10% CXB | — | — | — | — | — | — | — |
| control | Suspension | | | | | | | |
| 120 mb; hyd; | 3% 120 mb; | 120 | 3 | 10 | 0.3 | 3.33 | 1 | 10:3:10 |
| 27.8% cxbst; | 10% P188; | | | | | | | |
| 3% SFf; | 10% CXB | | | | | | | |
| 10% CXBf; | | | | | | | | |
| 10% P188f | | | | | | | | |
| 480 mb; hyd; | 3% 480 mb; | 480 | 3 | 10 | 0.3 | 3.33 | 1 | 10:3:10 |
| 27.8% cxbst; | 10% P188; | | | | | | | |
| 3% SFf; | 10% CXB | | | | | | | |
| 10% CXBf; | | | | | | | | |
| 10% P188f | | | | | | | | |

The corresponding solutions of excipients were prepared as described in Table 36. As with the hydrogels without CXB, a 2.87 mL volume of each excipient was aliquoted into a 3-cc syringe for subsequent mixing with the silk fibroin to generate the described formulation. For each sample, the syringe of the representative silk fibroin solution was connected to a syringe of its designated excipient solution via a B Braun fluid dispensing connector. The contents of the syringes were then mixed until homogenous. The resulting samples were incubated on a rotator for 24 hours at 37° C. and then separated into 0.4 mL aliquots in 1 cc syringes. The pH values of the samples were measured with a glass pH probe, and they were adjusted with hydrochloric acid. The resulting hydrogels had a ratio of silk fibroin to excipient of between 0.01 and 0.5, a ratio of celecoxib to silk fibroin of between 0.1 and 5, and a ratio of celecoxib to excipient of 1. The ratio of celecoxib to silk fibroin to excipient was 10:3:10. Formulations of the hydrogels contained sodium chloride, 0.2% (w/v) Polysorbate-80, and 22 mM phosphate buffer at pH=7.4 for the P188-containing hydrogels. Some formulations comprised 10% P188, 10% CXB, and 10.4 mg/mL sodium chloride at a pH of 7.4.

TABLE 36
Excipient solution preparations for hydrogels with celecoxib.

| | NaCl mg/mL | mL to | mL stock | μL 200 | μL DI |
|---|---|---|---|---|---|
| Sample Name | needed in exc. | make | P188 | mg/mL NaCl | Water |
| 120 mb; hyd; 27.8% | 10.44 | 4 | 3.48 | 208.8 | 311.2 |
| cxbst; 3% SFf; 10% | | | | | |
| CXBf; 10% P188f | | | | | |
| 480 mb; hyd; 27.8% | 10.36 | 4 | 3.48 | 207.2 | 312.8 |
| cxbst; 3% SFf; 10% | | | | | |
| CXBf; 10% P188f | | | | | |

### Administration of Hydrogels

The methods of administration of silk fibroin hydrogels with celecoxib were identical to those used to administer the hydrogels without celecoxib. Briefly, the subjects were New Zealand white rabbits with a mass of 4 kg. The rabbits were separated into three groups, with three rabbits in each group. Each group was injected with the formulation as described in Table 37. All injections were performed in the left eye, with the right eye remaining naïve to serve as an intra-animal control.

TABLE 37
Experimental groups of rabbits for the study of
silk fibroin hydrogels formulated with celecoxib.

| | | Name of Sample |
|---|---|---|
| Group | Description | Administered |
| 1 | 10% CXB Suspension | 10% CXB control |
| 2 | 3% HMW (120 mb) | 120 mb; hyd; 27.8% |
| | Silk; 10% Poloxamer- | cxbst; 3% SFf; 10% |
| | 188; 10% CXB | CXBf; 10% P188f |
| 3 | 3% LMW (480 mb) | 480 mb; hyd; 27.8% |
| | Silk; 10% Poloxamer- | cxbst; 3% SFf; 10% |
| | 188; 10% CXB | CXBf; 10% P188f |

All silk fibroin hydrogel formulations were pre-loaded into sterile 1 cc syringes, with 0.4 mL in each syringe. Prior to injection, the syringe cap was removed, and a sterile 27-gauges, ½% needle was attached. The volume was adjusted to 0.1 mL, and the formulation was injected into the intravitreal space, 2 mm posterior to the limbus. The method of injection was as described for the in vivo studies of silk fibroin hydrogels without celecoxib. Briefly, a lid speculum was inserted into the rabbit's left eye. The conjunctiva was rinsed with BSS solution. Then, the conjunctival sac was prepped with a 5% ophthalmic betadine solution. The hydrogel was then injected into the intravitreal space using the double panel technique described in the earlier in vivo studies of rods and gels. The formulation was delivered, and the needle was removed. Following injection, the central retinal artery was examined via indirect ophthalmoscopy to confirm perfusion and 1-2 drops of betadine solution were added to the conjunctiva prior to removal of the speculum.

All procedures were performed under general anesthesia. All rabbits had their peri-ocular fur of the left eye trimmed prior to the procedure. All rabbits received gentamycin ophthalmic ointment to the operative (left) eye in the recovery area post-procedure.

Intraocular Pressure and Biocompatibility after Injection of Hydrogels with Celecoxib

24 hours after the injection, and 7 days after the injection, the intraocular pressure was measured with a Tono-Pen, as shown in Table 38. Anterior penlight exams and posterior dilated fundus exams were also performed at these times. Even though larger animals, with a mass of approximately 4 kg, were used for this study than those used for the study of hydrogels without therapeutics, it was noted that hypoperfusion still occurred upon injection of 0.1 mL. This was expected as this volume was likely the largest volume that could be well-tolerated. Animal CCN-23 only received a half-volume injection and was therefore considered not usable for the current study. However, the injection did seem well-tolerated, and may be a suitable volume for injection in future studies. All hydrogel groups were more difficult to inject than their corresponding controls without drug. The hydrogels formed well defined, cohesive depots upon injection. These opaque formulations were easily visualized. Furthermore, the suspension, rather than immediately dispersing, stayed together well in the vitreous space.

TABLE 38
Intraocular pressure measurements at 24-hour exam with silk fibroin hydrogels
with celecoxib

| | | Left | | | | |
|---|---|---|---|---|---|---|
| | | Eye (Injected) | | Right Eye (Naïve) | | |
| | | Average | | Average | | |
| | | Left Eye | | Right | | Fold change in |
| | | IOP | Standard | Eye IOP | Standard | IOP |
| Group | Sample Name | (mmHg) | Deviation | (mmHg) | Deviation | (Injected/Naïve) |
| 1 | 10% CXB control | 10.00 | 4.58 | 11.33 | 1.53 | 0.88 |
| 2 | 120 mb; hyd; | 8.00 | 2.65 | 13.33 | 4.16 | 0.60 |
| | 27.8% cxbst; 3% SFf; | | | | | |
| | 10% CXBf; 10% P188f | | | | | |
| 3 | 480 mb; hyd; | 7.67 | 0.58 | 13.00 | 1.00 | 0.59 |
| | 27.8% cxbst; 3% SFf; | | | | | |
| | 10% CXBf; 10% P188f | | | | | |

24 hours after the injection, all animals exhibited slight conjunctival irritation. This result was attributed to the betadine solution used during the procedure. All silk hydrogel formulations, as well as the suspension, were physically unchanged. All formulations were determined to be well-tolerated via ocular examination. There were no observed signs of intraocular inflammation or irritation. Any slight hypoperfusion due to injection had resolved. Compared to the naïve contralateral eyes (the right eyes), no elevation in intraocular pressure (IOP) was measured with the Tono-Pen. In most cases, the IOP of the injected left eye was measured to be lower than that of the right eye (the control). The fold change of the intraocular pressure between the injected eye and the naïve eye decreased for all silk fibroin formulations relative to the CXB suspension control.

The eyes were examined again during a 7-day exam. The intraocular pressure was also measured at this timepoint, seen in Table 39A.

TABLE 39A
Intraocular pressure measurements at 7-day exam
with silk fibroin hydrogels with celecoxib

| | | Left | | | | |
|---|---|---|---|---|---|---|
| | | Eye (Injected) | | Right Eye (Naïve) | | |
| | | Average | | Average | | |
| | | Left Eye | | Right | | Fold change in |
| | | IOP | Standard | Eye IOP | Standard | IOP |
| Group | Sample Name | (mmHg) | Deviation | (mmHg) | Deviation | (Injected/Naïve) |
| 1 | 10% CXB control | 6.67 | 0.58 | 13.33 | 0.58 | 0.50 |
| 7 | 120 mb; hyd; | 7.67 | 2.31 | 10.00 | 4.36 | 0.77 |
| | 27.8% cxbst; 3% SFf; | | | | | |
| | 10% CXBf; | | | | | |
| | 10% P188f | | | | | |
| 3 | 480 mb; hyd; | 8.67 | 4.04 | 11.67 | 2.89 | 0.74 |
| | 27.8% cxbst; 3% SFf; | | | | | |
| | 10% CXBf; | | | | | |
| | 10% P188f | | | | | |

By the 7-day examination, all conjunctival irritation had subsided. The materials were concluded to be tolerated at 7 days. There were no obvious signs of inflammation. The hydrogels and the suspensions were cohesive at the 7-day timepoint. No elevation was detected in intraocular pressure compared to the naïve contralateral eyes. There was a slight trend toward lower intraocular pressures in the CXB-treated eyes. The fold change in the IOP between the injected and the naïve eye in each group was less than one, which indicated that all formulations reduced intraocular pressure. The fold change also revealed that the formulations with silk reduced the intraocular pressure to a lesser extent than the CXB suspension.

The analysis of the intraocular pressure was continued, as seen in Table 39B. At 4.5 months after administration, the CXB-containing hydrogels showed a slight decrease in intraocular pressure, similar to that of the CXB suspension. In addition, the intraocular pressure was measured to be the same as the untreated eye at 7 months after administration of hydrogel with no CXB. No local inflammation, hemorrhage, or other complications were detected 7 months after administration.

TABLE 39B
Continued measurements of intraocular pressure of silk
fibroin hydrogels with and without celecoxib

| | | Left Eye | | | | |
|---|---|---|---|---|---|---|
| | | (Injected) | | Right Eye (Naïve) | | Fold |
| | | Average | | Average | | change |
| | | Left Eye | Standard | Right Eye | Standard | in IOP |
| Sample | | IOP | De- | IOP | De- | (Injected/ |
| Name | Days | (mmHg) | viation | (mmHg) | viation | Naïve) |
| 10% CXB | 127 | 7.00 | 2.58 | 9.25 | 2.22 | 0.76 |
| control | | | | | | |
| 480 mb; hyd; | 127 | 6.00 | 2.16 | 7.75 | 3.30 | 0.77 |
| 27.8% cxbst; | | | | | | |
| 3% SFf; | | | | | | |
| 10% CXBf; | | | | | | |
| 10% P188f | | | | | | |
| 480 mb; hyd; | 197 | 10.33 | 3.21 | 10.67 | 3.21 | 0.97 |
| 27.8% cxbst; | | | | | | |
| 3% SFf; | | | | | | |
| 0% CXBf; | | | | | | |
| 10% P188f | | | | | | |

### Example 14. Histopathology Studies of Rabbit Eyes with Hydrogels

Following the experiments on intraocular pressure and biocompatibility, the animals were sacrificed, and both eyes were immediately enucleated and placed into a solution of 10% formalin. After 24 hours, the eyes were transferred to a solution of 70% ethanol for subsequent histopathology studies. Thirty-two rabbit eyes were submitted for the study. The eyes were processed into two blocks per sample. One slide per block was sectioned and stained with hematoxylin and eosin (H&E). The glass slides were evaluated by a board-certified veterinary pathologist via light microscopy. Histologic legions were graded for severity (0=absent; 1=minimal; 2=mild; 3=moderate; 4=marked; 5=severe).

Histologic findings in this study consisted of an infiltration of mixed inflammatory cells into the vitreous chamber, including heterophils (neutrophils), lymphocytes, plasma cells, macrophages and rare multinucleated giant cells. Inflammatory cells were primarily present in the region of the ora ciliaris retinae and variably surrounded presumed injected material within the vitreous chamber. This material ranged from basophilic flocculent to granular material, to more discrete, non-staining slightly refractile material less than 10 μm in diameter, to non-staining cleft-shaped material (resembling cholesterol clefts). Similar inflammatory cells infrequently extended into the adjacent ciliary body epithelium or retina. A granuloma, characterized by aggregation of macrophages and multinucleated giant cells, surrounding non-staining cholesterol cleft-like material and phagocytized debris, was present in the conjunctiva of one animal. Mononuclear inflammatory infiltrate was characterized by infiltration or aggregation of lymphocytes and plasma cells, with rare heterophils, in the conjunctiva. Infiltration of similar mononuclear cells into the iris was observed in one animal. Elevation of the retina from the retinal pigmented epithelium, present in many samples, was not associated with other features supportive of true retinal separation and this finding was therefore considered an artifact.

Means of the grades of the histologic lesions were examined, as well as the standard error of the mean (SEM), shown in Table 40. Mean scores for mixed inflammatory cell infiltration into the vitreous chamber were only observed in samples with intravitreal injections containing 10% celecoxib (CXB) (Groups 8-10). The highest mean score was observed in the 10% CXB suspension alone group (Group 8). The animal with a conjunctival granuloma was also in this group. Mean cores for conjunctival mononuclear cell infiltration severity were similar among all groups, regardless of injection status or injection material. Focal iris infiltration of inflammatory cells was only present in one animal, which had been from the low molecular weight (MW) solution group.

TABLE 40
H&E grades of the rabbit eye histopathology data of animals treated
with silk fibroin compositions (Standard error of the mean)

| | Name of | Inflammation, | | Infiltrate, | | | | Infiltrate, | |
|---|---|---|---|---|---|---|---|---|---|
| | injected | mixed, vitreous | | mononuclear, | | Granuloma, | | mononuclear, | |
| Group | sample | chamber | | iris | | conjunctiva | | conjunctiva | |
| Group 1 | Untreated | 0 | (±0.00) | 0 | (±0.00) | 0 | (±0.00) | 0.50 | (±0.50) |
| (Untreated) | | | | | | | | | |
| Group 2 (PBS) | PBS | 0 | (±0.00) | 0 | (±0.00) | 0 | (±0.00) | 0.33 | (±0.33) |
| Group 3 (HMW | 120 mb control | 0 | (±0.00) | 0 | (±0.00) | 0 | (±0.00) | 0.67 | (±0.33) |
| Solution) | | | | | | | | | |
| Group 4 (LMW | 480 mb control | 0 | (±0.00) | 0.33 | (±0.33) | 0 | (±0.00) | 0 | (±0.00) |
| Solution) | | | | | | | | | |
| Group 5 (3% | 120 mb; hyd; | 0 | (±0.00) | 0 | (±0.00) | 0 | (±0.00) | 1 | (±0.00) |
| HMW SF; 40% | 0% cxbst; 3% | | | | | | | | |
| 4 kDa PEG) | SFf; 0% CXBf; | | | | | | | | |
| | 40% PEG4kf | | | | | | | | |
| Group 6 (3% | 120 mb; hyd; | 0 | (±0.00) | 0 | (±0.00) | 0 | (±0.00) | 1.33 | (±0.33) |
| HMW SF, 10% | 0% cxbst; 3% | | | | | | | | |
| P188) | SFf, 0% CXBf; | | | | | | | | |
| | 10% P188f | | | | | | | | |
| Group 7 (3% | 480 mb; hyd; | 0 | (±0.00) | 0 | (±0.00) | 0 | (±0.00) | 1 | (±0.00) |
| LMW SF, 10% | 0% cxbst; 3% | | | | | | | | |
| P188) | SFf; 0% CXBf; | | | | | | | | |
| | 10% P188f | | | | | | | | |
| Group 8 (10% | 10% CXB control | 2 | (±0.00) | 0 | (±0.00) | 0.67 | (±0.67) | 1 | (±0.00) |
| CXB Suspension) | | | | | | | | | |
| Group 9 (3% | 120 mb; hyd; | 0.75 | (±0.48) | 0 | (±0.00) | 0 | (±0.00) | 0.75 | (±0.25) |
| HMW SF, 10% | 27.8% cxbst; | | | | | | | | |
| P188, 10% CXB | 3% SFf; 10% | | | | | | | | |
| Suspension) | CXBf; 10% | | | | | | | | |
| | P188f | | | | | | | | |
| Group 10 (3% | 480 mb; hyd; | 0.67 | (±0.33) | 0 | (±0.00) | 0 | (±0.00) | 0.67 | (±0.33) |
| LMW SF, 10% | 27.8% cxbst; | | | | | | | | |
| P188, 10% CXB | 3% SFf; 10% | | | | | | | | |
| Suspension) | CXBf; 10% | | | | | | | | |
| | P188f | | | | | | | | |

Imaging of an untreated eye displayed no lesions at the ora ciliaris retinae. The normal vitreous humor was visible as an acellular, slightly eosinophilic wispy material in the vitreous chamber. The ciliary body, retina, and sclera were also visible in the images. Imaging of an eye treated with a 10% CXB suspension demonstrated inflammatory infiltration into the vitreous chamber. There were more abundant heterophils, lymphocytes, and macrophages. Inflammatory cells were also rarely present in the retina.

Imaging of an eye treated with 120 mb; hyd; 0% cxbst; 3% SFf; 0% CXBf; 10% P188f showed that there was a mild infiltration of lymphocytes and mononuclear plasma cells within the conjunctiva.

Imaging of an eye treated with an intravitreal injection of 120 mb; hyd; 27.8% cxbst; 3% SFf; 10% CXBf; 10% P188f demonstrated that the injected vitreous material was more basophilic and granular compared to the normal vitreous humor. Macrophages, and fewer lymphocytes and heterophils, surrounded and infiltrated this material.

The major finding associated with intravitreal injections in this study was vitreous chamber mixed inflammation, limited to the eyes receiving injections containing 10% CXB. Mixed inflammatory cell infiltration in the vitreous chamber was only observed in groups receiving 10% CXB, with a 3-fold increase in the scores in the 10% CXB suspension group compared to groups 9 and 10 where the CXB was formulated with silk. This result showed that CXB silk formulations can potentially reduce the inflammatory responses seen with CXB only injections.

The observed inflammation was likely due to the presence of CXB. It is possible that the inflammation is a result of slight toxicity due to high initial levels of CXB in the vitreous. In the silk fibroin formulations, the initial levels of CXB in the vitreous were lower likely due to the slower release of the therapeutic agent. The inflammation might also have been caused by the suspension form of CXB. The smaller particles could induce a macrophage response; they could be engulfed by macrophages and ultimately lead to inflammation. By contrast, the hydrogel would contain these particles and reduce the resulting inflammation.

In most groups, there was minimal to mild conjunctival mononuclear infiltration. This inflammatory infiltrate typically targeted presumptive injected material, with a range in inflammatory response from primarily acute (heterophilic/neutrophilic) to a more foreign body-type reaction with more numerous macrophages ingesting the injected material. Extension of inflammatory cells into the surrounding tissues was infrequently present and was not associated with ciliary epithelial or retinal degeneration. The granuloma present in the conjunctiva of one eye (10% CXB Suspension group) was considered secondary to the injection procedure. Conjunctival and iridal mononuclear inflammatory cell infiltration was present in numerous eyes from both untreated and treated groups; these findings were considered background lesions that were unrelated to treatment. The retinal tissue was considered normal.

Additional histopathology studies were performed on animals sacrificed 6 and 7 months after administration of the silk fibroin hydrogels (480 mb; hyd; 0% cxbst; 3% SFf; 0% CXBf; 10% P188f). At 6 and 7 months after administration, the injected material was free of cellular infiltrate. No other histologic findings were observed. No local inflammation, hemorrhage, or other complications were observed. Ultimately the hydrogels were determined to be biocompatible and well-tolerated in the intravitreal space for at least 7 months after administration.

### Example 15. Histopathology Studies of Rabbit Eyes with Silk Rods

Following the experiments on intraocular pressure and biocompatibility, the animals were sacrificed, and both eyes were immediately enucleated and placed into a solution of 10% formalin. After 24 hours, the eyes were transferred to a solution of 70% ethanol for shipment and subsequent histopathology studies. The eyes were from animals sacrificed 1 week after administration of the silk rods. Four formalin-fixed rabbit eyes were processed into two blocks per sample. One slide per block was sectioned and stained with hematoxylin and eosin (H&E). The glass slides were evaluated by a board-certified veterinary pathologist, using light microscopy. Histologic lesions were graded for severity (0=absent; 1=minimal; 2=mild; 3=moderate; 4=marked; 5=severe), as seen in Table 41. L denoted the left eye, while R denoted the right eye.

TABLE 41
H&E grades of the rabbit eye histopathology data of animals treated with silk
fibroin rod compositions;

| | | | | H&E | | | |
|---|---|---|---|---|---|---|---|
| | | | | | Mixed | | |
| | | | | Foreign | infiltrate, | | |
| | | | | material, | vitreous | | |
| | | | | vitreous | chamber | | |
| | | | | chamber | (surrounding | | Mixed |
| Treat- | Sample | | | (presumptive | foreign | Degeneration, | inflammation, |
| ment | Name | Eye | Block | rod) | material) | lens fiber | conjunctiva |
| Silk- | 480 mb; | 56L | 1 | NP | 0 | 0 | 0 |
| Fibroin/ | 0.5 mm; | | 2 | P | 1 | 0 | 0 |
| CXB | 40% st; | 58L | 1 | P | 1 | 0 | 2 |
| Rod | 100 mgsf; | | 2 | P | 0 | 0 | 1 |
| | 200 mgcxb; | 59L | 1 | NP | 0 | 2 | 0 |
| | lyo; | | 2 | NP | 0 | 0 | 0 |
| | 33.3% sf; | | | | | | |
| | 66.7% cxb | | | | | | |
| Un- | — | 56R | 1 | NP | 0 | 0 | 0 |
| treated | | | 2 | NP | 0 | 0 | 0 |

P = present, NP = not present

Foreign material, presumably the injected celecoxib (CXB) rod, was present in the vitreous chamber of two eyes, near the ora ciliaris retinae. This material was a solid mass of amphophilic material, approximately 500 μm in diameter, containing non-staining clefts and vacuoles. This material was variably loosely surrounded or minimally infiltrated by low numbers of macrophages, rare heterophils and scant hemorrhage. Inflammation was not observed in other areas of the vitreous chamber or within the adjacent ciliary body/uveal tract or retina. In one eye, slight lens fiber degeneration was present. This finding might be associated with the injection procedure. Mixed inflammatory cell infiltration was observed in the conjunctiva from one eye. This finding was determined to be a background lesion, and it was unlikely to be associated with test article administration. Ultimately, histopathologic evaluation revealed minimal infiltration of low numbers of macrophages and rare heterophils. No other inflammation of note within the vitreous cavity, adjacent ciliary body, or retina, was detected. The silk rods were well tolerated in the intravitreal space.

Additional histopathology studies were performed on animals sacrificed 4 months after administration of the silk fibroin rods. The studies determined 2 out of the 3 rods to be acellular with visible implant. In 1 of the 3 rods the implant was surrounded and infiltrated by lymphocytes, macrophages, and multinucleated giant cells. Most of the samples did not illicit a significant inflammatory response. Ultimately the rods were determined to be biocompatible and well-tolerated in the intravitreal space for at least 4 months after administration.

### Example 16. Release of Protein Cargo and Relation of Release Kinetics to Protein Molecular Weight in Silk Fibroin Rods

Silk fibroin rods were prepared from silk fibroin degummed with a 480 mb or a 120 mb. Sodium chloride was purchased from Chemsavers (Bluefield Va.). Polysorbate-80 was purchased from Croda (Snaith, United Kingdom). Phosphate buffered saline (10×PBS) was purchased from Gibco (USA). Sodium phosphate dibasic, sodium phosphate monobasic, human lysozyme, sucrose, Bovine Serum Albumin (BSA), trehalose, and poloxamer-188 (P188) were purchased from Sigma-Aldrich (St. Louis, Mo.). Sodium azide and glycerol were purchased from Fisher Chemical (Waltham, Mass.). Bevacizumab was purchased from Genentech Inc. (San Francisco, Calif.). Human immunoglobulin G (IgG) was purchased from Innovative Research (Novi, Mich.).

Preparation of Silk Fibroin Rods with Proteins

Silk fibroin rods were formulated with proteins, and the controlled release of said proteins were monitored in vitro. Silk fibroin rods were formulated with lysozyme (molecular weight=14 kDa; Sigma-Aldrich, St. Louis, Mo.), bovine serum albumin (BSA) (molecular weight=67 kDa; Sigma-Aldrich, St. Louis, Mo.), bevacizumab (molecular weight=150 kDa; Genentech Inc., San Francisco, Calif.), and Immunoglobulin G (IgG) as described in Table 42. The aqueous processing of the silk fibroin rods was amenable to aseptic conditions. Some of the silk fibroin rods were 5% (w/w) of the respective protein. The silk fibroin rods are named by the process used to prepare and formulate each rod. For example, the rod named “480 mb; 1 mm; 5% bevst; lyo; 75% sf; 3% bevacizumab; 22% sucrose” refers to a rod prepared from silk degummed with a 480-minute boil (480 mb), a 1 mm diameter (1 mm), prepared from a 5% w/v bevacizumab stock solution (5% bevst), lyophilization (lyo), a theoretical w/w percentage of 75% silk fibroin (75% sf), a theoretical w/w percentage of 3% bevacizumab (3% bevacizumab), and a theoretical w/w percentage of 22% sucrose (22% sucrose). Other potential components of the rods described in the name included gelation at 4° C. (4° C.), a preparation from a stock solution of silk fibroin (e.g. 40% st), a theoretical w/w percentage of IgG (% igg), a theoretical w/w percentage of lysozyme (% lysozyme), a preparation from silk fibroin degummed with a 120-minute boil (120 mb), a preparation from silk fibroin degummed with a 90-minute boil (90 mb), a theoretical w/w percentage of bovine serum albumin (% bsa), and a theoretical w/w percentage of trehalose (% trehalose). Sample 205-1 contained 133.3 mM phosphate buffer. 205-2 contained 133.3 mM phosphate buffer. 205-5 contained 133.3 mM phosphate buffer. Rods with bevacizumab also contained small amounts of the buffer that the product was provided in (trehalose, a sodium phosphate buffer, and polysorbate-20).

TABLE 42
Silk rods formulated with proteins

| | | | | | | | | Sample |
|---|---|---|---|---|---|---|---|---|
| | | Time | Silk- | | | | | mass of |
| | | of | fibroin | | Protein | | | each |
| Sample | | heating | Conc. | | conc. | | Excipient | replicate |
| No. | Sample Name | (mb) | % | Protein | % | Excipient | Conc. % | (mg) |
| 204-05 | 480 mb; 1 mm; | 480 | 75 | Bevacizumab | 3 | Sucrose | 22 | 10.07 |
| | 5% bevst; lyo; | | | | | | | 9.82 |
| | 75% sf; | | | | | | | 9.74 |
| | 3% bevacizumab; | | | | | | | |
| | 22% sucrose | | | | | | | |
| 205-01 | 480 mb; 1 mm; | 480 | 85 | Bevacizumab | 5 | Sucrose | 10 | 9.71 |
| | 5% bevst; lyo; | | | | | | | 10.17 |
| | 85% sf; | | | | | | | 10.05 |
| | 5% bevacizumab; | | | | | | | |
| | 10% sucrose | | | | | | | |
| 205-02 | 480 mb; 1 mm; | 480 | 73 | Bevacizumab | 5 | Sucrose | 22 | 10.6 |
| | 5% bevst; lyo: | | | | | | | 9.87 |
| | 73% sf; | | | | | | | 10.36 |
| | 5% bevacizumab; | | | | | | | |
| | 22% sucrose | | | | | | | |
| 202-03 | 480 mb; 1 mm; | 480 | 95 | Bevacizumab | 5 | — | — | 6.18 |
| | 30% st; 5% bevst; | | | | | | | 7.16 |
| | lyo; 95% sf; | | | | | | | 6.93 |
| | 5% bevacizumab | | | | | | | |
| 205-04 | 480 mb; 1 mm; | 480 | 85 | IgG | 5 | Sucrose | 10 | 7.64 |
| | 40% st; 4° C.; lyo; | | | | | | | 8.3 |
| | 85% sf; 5% igg; | | | | | | | 7.7 |
| | 10% sucrose | | | | | | | |
| 205-05 | 480 mb; 1 mm; | 480 | 95 | Lysozyme | 5 | — | — | 6.68 |
| | lyo; 95% sf; | | | | | | | 7.8 |
| | 5% lysozyme | | | | | | | 6.22 |
| 205-06 | 480 mb; 1 mm; | 480 | 85 | Lysozyme | 5 | Sucrose | 10 | 8.66 |
| | lyo; 85% sf; | | | | | | | 7.94 |
| | 5% lysozyme; | | | | | | | 9.23 |
| | 10% sucrose | | | | | | | |
| 205-07 | 480 mb; 1 mm; | 480 | 75 | Lysozyme | 25 | — | — | 8.3 |
| | lyo; 75% sf; | | | | | | | 9.64 |
| | 25% lysozyme | | | | | | | — |
| 205-08 | 480 mb; 1 mm; | 480 | 65 | Lysozyme | 25 | Sucrose | 10 | 10.4 |
| | lyo; 65% sf; | | | | | | | 10.02 |
| | 25% lysozyme; | | | | | | | 7.98 |
| | 10% sucrose | | | | | | | |
| 205-A | 120 mb; 1 mm; | 120 | 95 | Lysozyme | 5 | — | — | 6.36 |
| | lyo; 95% sf; | | | | | | | 5.99 |
| | 5% lysozyme | | | | | | | 5.58 |
| 197-09 | 480 mb; 1 mm; | 480 | 96.5 | BSA | 2.5 | Trehalose | 1 | 9.24 |
| | 40% st; lyo; | | | | | | | 8.27 |
| | 96.5% sf; | | | | | | | 8.11 |
| | 2.5% bsa; | | | | | | | |
| | 1% trehalose | | | | | | | |
| 197-11 | 120 mb; 1 mm; | 120 | 96.5 | BSA | 2.5 | Trehalose | 1 | 4.89 |
| | 30% st; lyo; | | | | | | | 4.89 |
| | 96.5% sf; | | | | | | | 5.29 |
| | 2.5% bsa; | | | | | | | |
| | 1% trehalose | | | | | | | |
| 197-12 | 120 mb; 1 mm; | 120 | 94 | BSA | 5 | Trehalose | 1 | 6.59 |
| | 30% st; lyo; | | | | | | | 6 |
| | 94% sf; 5% bsa; | | | | | | | 6.02 |
| | 1% trehalose | | | | | | | |
| 201-04 | 480 mb; 1 mm; | 480 | 95 | Bevacizumab | 5 | — | — | 8.45 |
| | 5% bevst; 4° C.; | | | | | | | 8 |
| | lyo; 95% sf; | | | | | | | 7.89 |
| | 5% bevacizumab | | | | | | | |
| 209-05 | 90 mb; 1 mm; | 90 | 97.5 | IgG | 2.5 | — | — | — |
| | 30% st; 4° C.; lyo; | | | | | | | — |
| | 97.5% sf; | | | | | | | — |
| | 2.5% igg | | | | | | | |
| 209-A | 480 mb; 1 mm | 480 | 85 | IgG | 5 | Sucrose | 10 | — |
| | 40% st; 4° C.; lyo; | | | | | | | — |
| | 85% sf; 5% igg; | | | | | | | — |
| | 10% sucrose | | | | | | | |
| 191-01 | 480 mb; 1 mm; | 480 | 94 | BSA | 5 | Trehalose | 1 | — |
| | 40% st; lyo; | | | | | | | — |
| | 94% sf; 5% bsa; | | | | | | | — |
| | 1% trehalose | | | | | | | |
| 191-02 | 480 mb; 1 mm | 480 | 96.5 | BSA | 2.5 | Trehalose | 1 | — |
| | 40% st; lyo; | | | | | | | — |
| | 96.5% sf; | | | | | | | — |
| | 2.5% bsa; | | | | | | | |
| | 1% trehalose | | | | | | | |

To prepare the silk fibroin rods with lysozyme, silk fibroin was dissolved in lysozyme stock solution to reach the final desired silk/lysozyme concentrations. Sucrose (Sigma Aldrich, St. Louis Mo.) was dissolved in this solution when necessary. Formulations were injected into 1.0 mm diameter PTFE tubing. The tubing was capped with Parafilm® and allowed to gel at 37° C. overnight. Once gelling was achieved, the tubing was frozen and lyophilized.

To prepare the silk fibroin rods with BSA, silk fibroin was reconstituted in sufficient deionized water to reach a final concentration of 30 or 40% (w/v). BSA solutions were prepared, from a stock solution of 40 mg/mL BSA, with or without trehalose (Sigma Aldrich, St. Louis Mo.) and/or polysorbate-80 (Sigma Aldrich, St. Louis Mo.). Solutions were mixed between two syringes and extruded into 1.0 mm inner diameter PTFE tubing (Grainger, Ill., USA). The tubing was capped with Parafilm and allowed to gel at 4° C. overnight. Once gelling was achieved, the tubes were frozen and lyophilized. Samples 191-01 and 191-02 had 0.1% Tween-80 in the final formulation.

To prepare the silk fibroin rods with bevacizumab, silk fibroin was reconstituted in sufficient deionized water to reach a final concentration of 30% (Sample 202-03) or 40% (remaining samples) (w/v). The reconstituted fibroin was added to a concentrated solution of bevacizumab (50 mg/mL) to achieve the desired final ratio of bevacizumab:silk. Rods containing sucrose were prepared from silk fibroin lyophilized with sucrose. Solutions were mixed using two linked syringes and then injected into 1.0 mm diameter PTFE tubing. The rods were capped with Parafilm® and allowed to gel at 4° C. (Sample 201-4 only) or 37° C. overnight. Once gelling was achieved, the tubes were lyophilized overnight.

To prepare silk fibroin rods with immunoglobulin G (IgG), silk fibroin degummed with a 480 mb or a 90 mb, was reconstituted in sufficient deionized water to reach a final concentration of 30 or 40% (w/v). Rods containing sucrose were prepared from silk-fibroin lyophilized with sucrose as an additive. Solutions were mixed between two syringes and injected into 1.0 mm diameter PTFE tubing. The rods were capped with Parafilm®, and allowed to gel at 4° C. overnight. Once gelling was achieved, the tubes were frozen and lyophilized.

In Vitro Release Profile of Silk Fibroin Rods Formulated with Protein APIs

Silk fibroin rods were cut into 1 cm sections and two sections were placed, in triplicate, into 4 mL glass vials. 1 mL of release media (PBS, 0.01% polysorbate-80 (PS80), 0.05% sodium azide) was added to each vial. Samples were incubated with gentle shaking at 37° C. At 2 hours, 1, 2, 3, 7, 10, 14, 21, and 28 days, 100 μL of release media was removed and replaced with 100 μL of fresh release media. Total protein released was quantified via size-exclusion chromatography (SEC) using a Waters X-Bridge Protein BEH SEC, 200 Å, 3.5 μm column. An isocratic flow of mobile phase (100 mM sodium phosphate (Sigma Aldrich, St. Louis Mo.), 200 mM NaCl (Chemsavers, Bluefield Va.) pH 6.8) was run at 0.80 mL/min to elute protein. Protein elution was monitored at 280 and 214 nm using an Agilent 1290 HPLC system with a photodiode array (PDA) detector. Cumulative percentage of protein released was calculated using theoretical loading of the silk fibroin rods.

The average cumulative release percentage of each protein was monitored over time, as seen in Table 43A and Table 43B. The data suggested that release was related to size-dependent diffusion through the silk fibroin matrix. The release kinetics and the cumulative release percentages decreased with increased molecular weight of the protein to be released. Silk fibroin rods formulated with lysozyme had the highest initial burst percentage, while rods formulated with bevacizumab had the lowest initial burst percentage. The initial burst percentages ranged from 1-85% over the first 24 hours of the experiment. The cumulative release percentage of protein released from each rod were measured in triplicate, except for the specific measurements marked with “*”, which were measured in singlicate. Sample 205-07 and sample 197-12, marked with “***”, were tested in duplicate.

TABLE 43A
In vitro release of proteins from silk-fibroin rods; cumulative percentage (%) of API released

| Sample | | Days | | | | | |
|---|---|---|---|---|---|---|---|
| No. | Sample Name | 0 | 0.083 | 1 | 2 | 3 | 7 |
| 204-05 | 480 mb; 1 mm; 5% bevst; lyo; | 0.0 | 0.5 | 0.7 | — | — | — |
| | 75% sf; 3% bevacizmab; | | | | | | |
| | 22% sucrose | | | | | | |
| 205-01 | 480 mb; 1 mm; 5% bevst; lyo; | 0.0 | 6.6 | 6.9 | — | — | — |
| | 85% sf; 5% bevacizumab; | | | | | | |
| | 10% sucrose | | | | | | |
| 205-02 | 480 mb; 1 mm; 5% bevst; lyo; | 0.0 | 2.2 | 2.7 | — | — | — |
| | 73% sf; 5% bevacizumab; | | | | | | |
| | 22% sucrose | | | | | | |
| 202-03 | 480 mb; 1 mm; 30% st; | 0.0 | 3.2 | — | — | — | — |
| | 5% bevst; lyo; 95% sf; | | | | | | |
| | 5% bevacizumab | | | | | | |
| 205-04 | 480 mb; 1 mm; 40% st; 4° C.; | 0.0 | 5.7 | 19.4 | 20.2 | — | — |
| | lyo; 85% sf; 5% igg; | | | | | | |
| | 10% sucrose | | | | | | |
| 205-05 | 480 mb: 1 mm: lyo; 95% sf; | 0.0 | 18.7 | 27.2 | 32.4 | 7.2 | 7.6 |
| | 5% lysozyme | | | | | | |
| 205-06 | 480 mb; 1 mm; lyo; 85% sf; | 0.0 | 19.4 | 29.4 | 34.8 | *29.5 | 10.3 |
| | 5% lysozyme; 10% sucrose | | | | | | |
| 205-07*** | 480 mb; 1 mm; lyo; 75% sf; | 0.0 | 17.5 | 22.4 | 35.0 | 37.1 | 39.7 |
| | 25% lysozyme | | | | | | |
| 205-08 | 480 mb; 1 mm; lyo; 65% sf; | 0.0 | 48.7 | 75.0 | 83.3 | 71.1 | 73.9 |
| | 25% lysozyme; 10% sucrose | | | | | | |
| 205-A | 120 mb; 1 mm; lyo; 95% sf; | 0.0 | 11.6 | 12.8 | 14.2 | 10.3 | — |
| | 5% 1ysozyme | | | | | | |
| 197-09 | 480 mb; 1 mm; 40% st; lyo; | 0.0 | 0.0 | 0.0 | 0.0 | 0.0 | — |
| | 96.5% sf; 2.5% bsa; | | | | | | |
| | 1% trehalose | | | | | | |
| 197-11 | 120 mb; 1 mm; 30% st; lvo; | 0.0 | 13.7 | 21.3 | 21.6 | 26.1 | — |
| | 96.5% sf; 2.5% bsa.; | | | | | | |
| | 1% trehalose | | | | | | |
| 197-12*** | 120 mb; 1 mm; 30% st; lyo; | 0.0 | 9.1 | 14.2 | 16.0 | 17.9 | — |
| | 94% sf; 5% bsa; 1% trehalose | | | | | | |
| 201-04 | 480 mb; 1 mm; 5% bevst; 4° C.; | 0.0 | 56.0 | 84.5 | 73.8 | 58.3 | 66.8 |
| | lyo; 95% sf; 5% bevacizumab | | | | | | |

TABLE 43B
Standard deviations (%) of the cumulative percentage
of API released for the in vitro release
of proteins from silk rods

| Sample | | Day | | | | | |
|---|---|---|---|---|---|---|---|
| No. | Sample Name | 0 | 0.083 | 1 | 2 | 3 | 7 |
| 204-05 | 480 mb; 1 mm; 5% bevst; | 0.0 | 0.1 | 0.2 | — | — | — |
| | lyo; 75% sf; | | | | | | |
| | 3% bevacizumab; | | | | | | |
| | 22% sucrose | | | | | | |
| 205-01 | 480 mb; 1 mm; 5% bevst; | 0.0 | 2.7 | 2.9 | — | — | — |
| | lyo; 85% sf; | | | | | | |
| | 5% bevacizumab; | | | | | | |
| | 10% sucrose | | | | | | |
| 205-02 | 480 mb; 1 mm; 5% bevst; | 0.0 | 1.0 | — | — | — | — |
| | lyo; 73% sf; | | | | | | |
| | 5% bevacizumab; | | | | | | |
| | 22% sucrose | | | | | | |
| 202-03 | 480 mb; 1 mm; 30% st; | 0.0 | 0.4 | — | — | — | — |
| | 5% bevst; lyo; | | | | | | |
| | 95% sf; 5% bevacizumab | | | | | | |
| 205-04 | 480 mb; 1 mm; 40% st; | 0.0 | 0.3 | 0.9 | 0.8 | — | — |
| | 4° C.; lyo; 85% sf; | | | | | | |
| | 5% igg; 10% sucrose | | | | | | |
| 205-05 | 480 mb; 1 mm; lyo; 95% sf; | 0.0 | 3.3 | 1.2 | 2.6 | 0.6 | 0.7 |
| | 5% lysozyme | | | | | | |
| 205-06 | 480 mb: 1 mm; lyo; 85% sf; | 0.0 | 1.0 | 1.6 | 1.1 | *0.0 | 1.2 |
| | 5% lysozyme; 10% sucrose | | | | | | |
| 205-07*** | 480 mb; 1 mm; lyo; 75% sf; | 0.0 | 2.9 | 3.0 | 2.5 | 2.9 | 4.5 |
| | 25% lysozyme | | | | | | |
| 205-08 | 480 mb; 1 mm; lyo; 65% sf; | 0.0 | 1.1 | 2.6 | 2.8 | 6.1 | 1.8 |
| | 25% lysozyme; 10% sucrose | | | | | | |
| 205-A | 120 mb; 1 mm; lyo; 95% sf; | 0.0 | 1.1 | 1.1 | 0.3 | 0.1 | — |
| | 5% lysozyme | | | | | | |
| 197-09 | 480 mb; 1 mm; 40% st; | 0.0 | 6.0 | 0.0 | 0.0 | 0 0 | — |
| | lyo; 96.5% sf; | | | | | | |
| | 2.5% bsa; 1% trehalose | | | | | | |
| 197-11 | 120 mb; 1 mm; 30% st; | 0.0 | 1.8 | 3.0 | 8.6 | 5.6 | — |
| | lyo; 96.5% sf; | | | | | | |
| | 2.5 bsa; 1% trehalose | | | | | | |
| 197-12*** | 120 mb; 1 mm; 30% st; | 0.0 | 0.5 | 0.2 | 2.2 | 1.1 | — |
| | lyo; 94% sf; | | | | | | |
| | 5% bsa 1% trehalose | | | | | | |
| 201-04 | 480 mb; 1 mm; 5% bevst; | 0.0 | 2.5 | 3.4 | 2.3 | 3.0 | 2.5 |
| | 4° C.; lyo; | | | | | | |
| | 95% sf; 5% bevacizumab | | | | | | |

Silk fibroin molecular weight seemed to play a role in release of lysozyme from silk fibroin rods. Increasing the silk fibroin molecular weight from low molecular silk fibroin (480 mb) to relatively higher molecular weight silk fibroin (120 mb), with 5% lysozyme loading as seen in samples 205-05 and 205-A respectively, decreased the initial burst and cumulative release percentage over 3 days.

BSA-containing rods with lower molecular weight silk fibroin (480 mb) showed a protein-loading dependent release. Rods prepared from 480 mb silk fibroin with 2.5% BSA showed release below detectable levels (BDL) out to 3 days (197-09). Rods prepared from 120 mb silk fibroin with low loading (2.5% BSA, sample 197-11) showed faster release kinetics in comparison with the corresponding rods with higher BSA loading (197-12). The lower loaded 120 mb rods (197-11) initial burst at 2 hours of 13.7% and a cumulative release of 26.1% by day 3. 120 mb silk fibroin rods showed faster release of BSA than the comparable formulation made with 480 mb silk fibroin (which showed no release). The results suggested a relationship between the BSA:silk fibroin ratio and the release kinetics of the protein from the rod.

For the silk fibroin rods prepared with bevacizumab, all formulations showed very little burst (less than or equal to 7%) with no continued release, with the exception of the rod formulation prepared at 4° C. (201-04). This low temperature rod had a burst at 2 hours of 56.0% of the loaded protein, with 84.5% of the protein released after 1 day This formulation temperature-dependent release could be caused by an increase in non-specific or hydrophobic binding of silk fibroin and bevacizumab at elevated temperatures. The lower temperature might also effect the tightness and size of the silk fibroin network within the rod formulation.

The silk fibroin rod with IgG subject to the in vitro experiments (205-04, 480 mb; 1 mm; 40% st; 4° C.; lyo; 85% sf, 5% igg; 10% sucrose) showed a lower burst and release out to 2 days. 2 hours into the experiment, 5.7% of the protein was released, and the cumulative release percentage leveled after 1 day at about 19.4%. This rod released more protein than similar rods with 5% bevacizumab (205-01), but it released less protein than similar rods with 5% lysozyme (205-06).

The release data from 5% analyte rod formulations for lysozyme (205-05), BSA (197-12), and bevacizumab (202-03 and 205-01) demonstrated a trend. The smaller proteins, lysozyme and BSA, had higher burst releases from the rods and faster release kinetics than bevacizumab. Additionally, the rods formulated with smaller proteins seemed to release protein over several days, whereas release of bevacizumab (a larger molecule) for the rod formulation plateaued after 1 day of release.

### Example 17. Excipient Effects on Release Kinetics of Protein Cargo

Silk fibroin rods were formulated with proteins, and the controlled release of said proteins were monitored in vitro. Silk fibroin rods were formulated with 5 or 25% (w/w) lysozyme (molecular weight=14 kDa). Some silk fibroin rods were formulated with 5 or 25% (w/w) lysozyme, and with 10% (w/w) sucrose as an excipient. The excipient was added to reduce the silk concentration, while increasing the size of the silk fibroin network and tuning the release kinetics.

Silk fibroin rods were prepared from silk fibroin degummed with a 480 mb. Sodium chloride was purchased from Chemsavers (Bluefield Va.). Polysorbate-80 was purchased from Croda (Snaith, United Kingdom). Phosphate buffered saline (10×PBS) was purchased from Gibco (USA). Sodium phosphate dibasic, sodium phosphate monobasic, human lysozyme, sucrose, were purchased from Sigma-Aldrich (St. Louis, Mo.). Sodium azide and glycerol were purchased from Fisher Chemical (Waltham, Mass.).

Preparation of Silk Fibroin Rods with Proteins and Other Excipients

To prepare the silk fibroin rods with lysozyme, silk fibroin was dissolved in lysozyme stock solution to reach the final desired silk/lysozyme concentrations. Sucrose (Sigma Aldrich, St. Louis Mo.) was dissolved in this solution when necessary. Formulations were injected into 1.0 mm diameter PTFE tubing. The tubing was capped with Parafilm and allowed to gel at 37° C. overnight. Once gelling was achieved, the tubing was frozen and lyophilized. The formulations were prepared as described in Table 44. The silk fibroin rods are named by the process used to prepare and formulate each rod. For example, the rod named 480 mb; 1 mm; lyo; 85% sf; 5% lysozyme; 10% sucrose refers to a rod prepared with silk degummed with a 480-minute boil (480 mb), a 1 mm diameter (1 mm), lyophilization (lyo), a theoretical w/w percentage of 85% silk fibroin (85% sf), a theoretical w/w percentage of 5% lysozyme (5% lysozyme), and a theoretical w/w percentage of 10% sucrose (10% sucrose). Sample 205-05 also contained 133.3 mM phosphate buffer.

TABLE 44
Silk rods formulated with proteins and exeipients

| | | Time of | Silk- | | | | |
|---|---|---|---|---|---|---|---|
| Sample | | heating | fibroin | | Protein | | Excipient |
| No. | Sample name | (mb) | Conc. % | Protein | conc. % | Excipient | conc. % |
| 205-05 | 480 mb; 1 mm; lyo; | 480 | 95 | Lysozyme | 5 | — | — |
| | 95% sf; 5% lysozyme | | | | | | |
| 205-06 | 480 mb; 1 mm; lyo; | 480 | 85 | Lysozyme | 5 | Sucrose | 10 |
| | 85% sf; 5% lysozyme; | | | | | | |
| | 10% sucrose | | | | | | |
| 205-07 | 480 mb; 1 mm; lyo; | 480 | 75 | Lysozyme | 25 | — | — |
| | 75% sf; 25% lysozyme | | | | | | |
| 205-08 | 480 mb; 1 mm; lyo; | 480 | 65 | Lysozyme | 25 | Sucrose | 10 |
| | 65% sf; 25% lysozyme; | | | | | | |
| | 10% sucrose | | | | | | |

In Vitro Release Profile of Silk Fibroin Rods Formulated with Protein APIs and Other Excipients

Silk fibroin rods were cut into 1 cm sections and two sections were placed, in triplicate, into 4 mL glass vials. 1 mL of release media was added to each vial. Samples were incubated with gentle shaking at 37° C. At 2 hours, 1, 2, 3, 7, 10, 14, 21, and 28 days, 100 μL of release media was removed and replaced with 100 μL of fresh release media. Total protein released was quantified via size-exclusion chromatography (SEC) using a Waters X-Bridge Protein BEH SEC, 200 Å, 3.5 μm column. An isocratic flow of mobile phase (100 mM sodium phosphate (Sigma Aldrich, St. Louis Mo.), 200 mM NaCl (Chemsavers, Bluefield Va.) pH 6.8) was run at 0.80 mL/min to elute protein. Protein elution was monitored at 280 and 214 nm using an Agilent 1290 HPLC system with a PDA detector. Cumulative percentage of protein released was calculated using theoretical loading of the silk fibroin rods.

The cumulative release percentage of each protein was monitored over time, as seen in Table 45A and Table 45B. The incorporation of sucrose in the silk fibroin rods resulted in a faster release of lysozyme for some of the rod formulations. The initial burst of lysozyme release was at least two-fold greater for the rods formulated with sucrose and 25% lysozyme. Furthermore, the cumulative release percentage of lysozyme was at least about two-fold greater over time when the rods were formulated with sucrose and 25% lysozyme. The cumulative release percentage of protein released from each rod were measured in triplicate, except for the specific measurements marked with “*”, which were measured in singlicate. Sample 205-07, marked with “***”, was tested in duplicate.

TABLE 45A
In vitro release of Lysozyme from silk-fibroin rods with and
without an excipient; cumulative percentage (%) of API released

| Sample | Day | | | | | |
|---|---|---|---|---|---|---|
| No. | 0 | 0.083 | 1 | 2 | 3 | 7 |
| 205-05 | 0.00 | 18.74 | 27.19 | 32.43 | 7.21 | 7.64 |
| 205-06 | 0.00 | 19.42 | 29.37 | 34.84 | *29.54 | 10.29 |
| 205-07*** | 0.00 | 17.50 | 22.42 | 35.04 | 37.12 | 39.75 |
| 205-08 | 0.00 | 48.69 | 74.98 | 83.25 | 71.14 | 73.94 |

TABLE 45B
Standard deviation of in vitro release of Lysozyme from
silk-fibroin rods with and without an excipient;
in terms of cumulative percentage (%) of API released

| Sample | Day | | | | | |
|---|---|---|---|---|---|---|
| No. | 0 | 0.083 | 1 | 2 | 3 | 7 |
| 205-05 | 0.00 | 3.25 | 1.24 | 2.63 | 0.56 | 0.71 |
| 205-06 | 0.00 | 1.00 | 1.61 | 1.09 | 0.00* | 1.24 |
| 205-07*** | 0.00 | 7.85 | 2.98 | 2.47 | 2.87 | 4.52 |
| 205-08 | 0.00 | 1.13 | 2.60 | 2.80 | 6.11 | 1.85 |

Silk fibroin rods loaded with 5% lysozyme (sample 205-05) had similar release profiles to rods loaded with 25% lysozyme (205-07). However, the addition of sucrose affected these formulations very differently. Replacing 10% silk fibroin with sucrose did not change the 5% lysozyme loaded formulation release, while it increased the initial burst (measured at 2 hours) of the 25% lysozyme rod from 17.5% to 48.7%. This result suggested a critical silk fibroin:lysozyme ratio that needed to be maintained to reduce the initial burst. Adding sucrose in place of silk fibroin reduced this ratio enough in the higher loaded lysozyme rods, but not in the rods with lower loading.

### Example 18. In Vivo Ocular Pharmacokinetic Studies with Silk Fibroin Rods and Hydrogels with Celecoxib

Silk fibroin platforms were evaluated for delivery of celecoxib (CXB) to the intraocular tissues. Both the hydrogel and rod formulations were well tolerated, showing no negative clinical symptoms, rise in intraocular pressure (IOP), or adverse histological findings over 6 months. After the silk fibroin rods or 0.050 mL samples of hydrogels were administered, the SBPs were subject to pharmacokinetic studies. Multiple preparations of the same formulations were used. The average calculated CXB dose for the hydrogels comprised 3.5-3.6 mg, while the average calculated CXB dose comprised 0.59 to 0.75 mg for the rods. Clinical exams, intraocular pressure (IOP), and histological assessment were performed to determine local tolerability. Vitreous humor (VH) and retina/choroid (RC) tissues were collected and analyzed for CXB concentration over 6 months. Animals had gross examinations of the eye as well as slit-lamp fundus examinations. For slit-lamp exams, a hand-held slit-lamp (Koma or similar) were used.

Briefly, the concentration of API in the vitreous humor was determined after the administration of CXB via silk fibroin rod. After the in vivo silk rods experiments, the vitreous humor of the subjects of the experiments was analyzed for the concentration of celecoxib present. The silk fibroin rods (480 mb; 0.5 mm; 40% st; 100mgsf; 200mgcxb; lyo; 33.3% sf; 66.7% cxb) and silk fibroin hydrogels were administered to the left eye of New Zealand white rabbits, with a total celecoxib dose of 640-750 μg. Two to three animals were used in each group for each time point. The rabbits were sacrificed at about 2 weeks, 1 month 2 months, 3 months, 4.5 months, and 6 months after injection.

### Formulation Residence Time

The formulations containing celecoxib were still clinically visible at 6 months post injection (10% CXB suspension, 10% CXB hydrogel, and CXB rod). All hydrogel and suspension groups had reduced in size over time. Additionally, the 1.4% CXB suspension was visible clinically out to 3 months. A blank hydrogel formulation was evaluated out to 7 months, and although it decreased in size, it was still clinically present at the time of sacrifice. Formulations had no adverse clinical findings for the duration of the study.

### Celecoxib Detection in Aqueous Humor

The concentration of API in the aqueous humor was determined after the administration of CXB with different API delivery media. To collect the aqueous humor, the animals were anesthetized. Approximately 50-100 μL aqueous humor was removed from the anterior chamber at the limbus by a 31G needle attached to a 1 mL insulin syringe. Samples of the aqueous humor were prepared in a 50/50 Acetonitrile/50 mM Ammonium Formate, pH 4.0 buffer and analyzed via HPLC. The results of the in vivo administration of celecoxib through the eye were shown in Table 46. As seen in the Table 46, at least 50% of the animals subject to experiments with silk fibroin rods had detectable amounts of CXB in the aqueous humor after 7 days. 100% of the animals tested with silk fibroin rods had detectable levels of CXB in the aqueous humor after 28 days.

TABLE 46
Detection and concentration of celecoxib in the
aqueous humor after intraocular administration

| | | | | | | % of Animals |
|---|---|---|---|---|---|---|
| | | | | Average | | with |
| | CXB | | | Concentration | St. | Detectable |
| Sample Name | Dose | | Day | (ng/mL) | Dev. | CXB |
| 10% CXB Control | 5 | mg | 7 | 0.43 | 0.45 | 100 |
| | | | 28 | 1.25 | 1.09 | 100 |
| | | | 56 | 0.58 | 0.40 | 100 |
| 480 mb; hyd; 27.8% | 5 | mg | 7 | 1.03 | 1.66 | 83.3 |
| cxbst; 3% SFf; 10% | | | 28 | 1.17 | 0.49 | 100 |
| CXBf; 10% P188f | | | 56 | 1.62 | 0.40 | 100 |
| 480 mb; 0.5 mm; 40% | 0.7 | mg | 7 | 0.27 | 0.15 | 50 |
| st; 100 mgsf; 200 | | | 28 | 0.38 | 0.30 | 100 |
| mgcxb; lyo; 33.3% | | | 56 | 0.30 | 0.07 | 100 |
| sf; 66.7% cxb | | | | | | |

### Celecoxib Detection in Whole Eye

The animals were euthanized, and eyes were enucleated and immediately snap frozen in liquid nitrogen, position of the implant/formulation was visualized and recorded to ensure that each eye was oriented appropriately during freezing and dissection. The eyes were then bisected ensuring that the implant/formulation was completely retained in one half of vitreous. The eyes were then thawed, and both vitreous hemispheres (formulation and no formulation) were collected. The vitreous with no formulation was analyzed for CXB concentration via HPLC-MS. The vitreous containing the formulation was centrifuged at 10,000×g for 10 minutes. The supernatant was removed and analyzed for CXB concentration via HPLC-MS. Samples of the vitreous humor were prepared in a 50/50 Acetonitrile/50 mM Ammonium Formate, pH 4.0 buffer prior to analysis via HPLC. The formulation pellet collected after centrifugation was frozen and lyophilized. CXB was extracted from the formulations using acetonitrile and analyzed via HPLC-UV.

Furthermore, the retina and choroid were dissected from both hemispheres for extraction and analysis via HPLC-MS. Samples of retinoid were initially wetted with acetonitrile and dried prior to sample preparation. The retinoid samples were finely cut with a scissors and mixed into a uniform paste. 10 times the weight of 50/50 Acetonitrile/50 mM ammonium formate pH 4 was added to every sample. The samples were then vortexed for 2 minutes, sonicated for 15 minutes, and refrigerated overnight. The samples were then sonicated for an additional 15 minutes, then centrifuged for 8 minutes and then processed per the same test procedures used for the aqueous and vitreous humors.

The concentration of celecoxib in the vitreous humor from each bisected half (with and without the implanted silk fibroin rod) was analyzed, as seen in Table 47A and Table 47B. At each timepoint, the concentration of celecoxib in the vitreous humor, with and without the implant, was determined to be greater than or equal to the IC₅₀, the half-maximal inhibitory concentration, of celecoxib which was 40 nM (15.3 ng/mL). The silk fibroin rods showed near steady state drug concentrations with concentrations in the vitreous humor greater than or equal to the IC₅₀ of or three months. Controls of celecoxib suspensions ere also analyzed, with an approximate dosage of 5 mg celecoxib.

TABLE 47A
Descriptions of samples analyzed for concentrations of celecoxib in whole eye

| | | CXB | | CXB | | CXB | CXB |
|---|---|---|---|---|---|---|---|
| | | Theoretical | | Average | | Theoretical | Average |
| Sample | Sample Name | Dose | | Dose | | loading | Loading |
| Low CXB | 1.4% CXB | 0.7 | mg | 0.65 | mg | 1.4% | 1.3% |
| control | Suspension | (14 | mg/mL) | | | | |
| High CXB | 10% CXB | 5 | mg | 4.0-4.3 | mg | 10% | 8.0-8.5% |
| control | Suspension | (100 | mg/mL) | | | | |
| 10% CXB | 480 mb; hyd; | 5 | mg | 3.5-3.6 | mg | 10% | 6.9-7.2% |
| hydrogel | 27.8% cxbst; | (100 | mg/mL) | | | | |
| | 3% SFf; 10% | | | | | | |
| | CXBf; 10% | | | | | | |
| | P188f | | | | | | |
| CXB rods | 480 mb; 0.5 | 0.7 | mg | 0.59-0.75 | mg | 66.7% | 44.7-52.5% |

| | |
|---|---|
| mm; 40% st; | (N/A) |
| 100 mgsf; 200 | |
| mgcxb; lyo; | |
| 33.3% sf; | |
| 66.7% cxb | |

TABLE 47B
Detection and concentration of celecoxib in the vitreous humor (VH) and retina
after intraocular administration

| | | | | VH No | | VH | | Retina/ | |
|---|---|---|---|---|---|---|---|---|---|
| | Sample | CXB | | Implant | Std | Implant | Std | Choroid | Std |
| Sample | Name | Dose | Day | (ng/mL) | Dev. | (ng/mL) | Dev. | (ng/mL) | Dev. |
| Low | 1.4% CXB | 0.7 mg | 14 | 817 | 690 | 17733 | 29503 | 4190 | 4587 |
| CXB | Suspension | (14 mg/mL) | 29 | 100 | 129 | 28806 | 39590 | 65 | 15 |
| control | | | 84 | 17 | 12 | 3445 | 4992 | 55 | 48 |
| High | 10% CXB | 45 mg | 14 | 491 | 787 | 434 | 665 | 36338 | 53177 |
| CXB | Suspension | (100 mg/mL) | 86 | 11 | 2 | 7125 | 7036 | 131 | 37 |
| control | | | 127 | 133 | 161 | 1173 | 462 | 141 | 37 |
| | | | 170 | 1998 | 2760 | 834 | 914 | 1194 | 154 |
| 10% | 480 mb; | 5 mg | 14 | 4663 | 7314 | 12167 | 12262 | 7349 | 11480 |
| CXB | hyd; | (100 mg/mL) | 86 | 3807 | 5124 | 18050 | 3182 | 60400 | 27153 |
| hydrogel | 27.8% cxbst; | | 127 | 125 | 144 | 708 | 165 | 344 | 161 |
| | 3% SFf; | | 170 | 24 | 4 | 1314 | 1353 | 122 | 81 |
| | 10% CXBf; | | | | | | | | |
| | 10% P188f | | | | | | | | |
| CXB | 480 mb; | 0.7 mg | 14 | 70 | 91 | 413 | 441 | 133 | 99 |
| rods | 0.5 mm; | (N/A) | 29 | 11832 | 16501 | 220 | 13 | 1254 | 1635 |
| | 40% st; | | 58 | 25 | 21 | 317 | 80 | 1493 | 986 |
| | 100 mgsf; | | 86 | 31 | 18 | 783 | 774 | 60 | 46 |
| | 200 mgcxb; | | 126 | 30 | 24 | 170 | 97 | 79 | 39 |
| | lyo; | | 169 | 45 | 1 | 234 | 4 | 159 | 11 |
| | 33.3% sf; | | | | | | | | |
| | 66.7% cxb | | | | | | | | |

At 14 days, the low concentration suspension formulations exhibited comparatively lower CXB concentrations in the vitreous with no formulation, while the vitreous with formulation as well as the retina/choroid had higher concentrations of CXB. This may have been due to the nature of the suspension formulations, which are more diffuse within the vitreous humor and more difficult to separate than the silk fibroin formulations. The vitreous humor containing the formulation ranged from 28806 ng/mL to 3445 ng/mL CXB, maintaining levels well above the estimated EC₈₀ for celecoxib (1-3 μM; 381-1143 ng/mL). The vitreous humor with no formulation as well as the retina/choroid showed very similar trends of high concentration at 14 days followed by a dramatic drop by 30 days. This low level was decreased further out to 90 days. The intravitreal concentration of CXB generally decreased over the 84 day time frame with the administration of the 1.4% CXB suspension. CXB concentrations in multiple tissues fell below the EC₈₀ by 29 days and approached the reported biochemical inhibitory concentration (IC50; 40 nM; 15 ng/mL) by 90 days post injection.

The intravitreal injection of a 10% CXB suspension showed decreasing retinal tissue concentrations from 14 to 86 days (36338 ng/mL to 131 ng/mL). This concentration was then maintained in the retina/choroid over 6 months at 130-200 ng/mL (below the EC₈₀ for celecoxib). Vitreous humor CXB concentrations displayed differences over time which seemed to be dependent on the hemisphere. Over the 170 day experiment, the concentration of CXB delivered by the 10% CXB suspension, was variable amongst the tissues. After injection of the 10% CXB suspension, both vitreous halves had similar CXB concentrations at 14 days (491 ng/mL and 433 ng/mL for no formulation and formulation vitreous respectively); however, these two locations varied more noticeably at the later timepoints (86 days or longer). The vitreous humor containing the formulation showed a maximum CXB concentration of 7125 ng/mL at 3 months, which then decreased to approximately 1000 ng/mL after 127 days. The vitreous humor from the hemisphere containing no formulation dropped to a concentration of only 11 ng/mL at about 3 months, then increased at 127 and 170 days to 133 ng/mL and 1998 ng/mL. This variability, similar to the lower concentration suspension group, may have been due to the dispersity of the suspension and inefficient removal of undissolved CXB during extraction. Although all of the tissues displayed levels at or above the EC₈₀ for CXB at 14 days, only the vitreous humor containing the formulation maintained concentrations in this range over the 6 months of the study. CXB concentrations in the other tissues fell well below this concentration by 3 months.

The silk-fibroin hydrogel formulation containing 10% CXB (5 mg dose) displayed elevated, steady-state concentrations in both vitreous samples as well as retina/choroid tissue over 86 days, which decreased slightly thereafter. The retina/choroid showed CXB levels of 7349 ng/mL and 60400 ng/mL (7 times and 60 times the EC₈₀ for CXB) at 14 days and 86 days, respectively. Concentrations decreased to 344 ng/mL at 127 days (within the EC₈₀) and further to 122 ng/mL at about 6 months. Vitreous humor containing the formulation maintained levels at or above the ECs for the duration of the study. Over the first 3 months, concentrations ranged slightly from 12167-18050 ng/mL CXB. These concentrations decreased to 708 ng/mL and 1314 ng/mL at 127 and 170 days. The vitreous humor with no formulation was also well above the EC₅₀ over the first 3 months with concentrations in the range of 3807-4663 ng/mL. Similar to the other tissues, CXB concentrations decreased at about 4.5 and 6 months, however these CXB levels fell below the EC₈₀. The hydrogels maintained higher local levels of CXB over the course of the study. These concentrations were above the ICs for CXB to COX-2, as described in Table 48. During the 6 months of the study all tissue concentrations for the hydrogel formulation were maintained well above the IC₅₀ for CXB.

Silk-fibroin rod implant formulations loaded with CXB exhibited steady-state drug levels in the vitreous as well as retina/choroid above the ICs for CXB to COX-2 for greater than 3 months, and at least 169 days. Silk-fibroin rod implant formulations loaded with CXB exhibited steady-state drug levels in the vitreous humor as well as retina/choroid above the IC₅₀ for CXB to COX-2 for 6 months. Data showed that the CXB concentration in the two vitreous humor samples trended together with the same steady-state. However, in most cases there was 5-10 times higher CXB concentration throughout the study in the hemisphere containing the implant, displaying a CXB concentration gradient. Individual timepoints at 14 days, about 2, about 3, about 4, and about 6 months indicated that the CXB concentration in vitreous humor was higher in the hemisphere containing the implant. In the vitreous humor containing the implant, CXB levels ranged from 170 ng/mL to 783 ng/mL over the 6 months evaluated, with the highest concentration recorded at 86 days. These concentrations were very close to the expected EC₅₀ for CXB. Drug levels in the opposing vitreous humor hemisphere, however, dipped below this mark and ranged from 25 ng/mL to 70 ng/mL, with an exception of 11832 ng/mL at about 1 month. Retina/choroid tissue showed a spike in CXB concentration of 1254 and 1493 ng/mL at 29 and 58 days respectively, bringing the levels above the efficacious range (EC₅₀). CXB concentrations in the retina/choroid at 14 days and about 3-6 months were lower and very steady, ranging from only 60 ng/mL to 159 ng/mL.

TABLE 48
Fold increase of concentration of celecoxib in the eye over IC₅₀ of celecoxib with
COX-2 (In vivo API concentration/IC₅₀ of celecoxib)

| | | | | Fold over IC50 (15 ng/mL) (API | | |
|---|---|---|---|---|---|---|
| | | | | concentration/IC50 of API with COX-2) | | |
| | | CXB | | VH No | VH | |
| Sample | Sample Name | Dose | Day | Implant | Implant | Retina/Choroid |
| Low CXB | 1.4% CXB | 0.7 mg | 14 | 54.5 | 1182.2 | 279.3 |
| control | Suspension | | 29 | 6.6 | 1920.4 | 4.4 |
| | | | 84 | 1.2 | 229.6 | 3.7 |
| High CXB | 10% CXB | 4 mg | 14 | 32.7 | 28.9 | 2422.5 |
| control | Suspension | | 86 | 0.7 | 475.0 | 8.7 |
| | | | 127 | 8.9 | 78.2 | 9.4 |
| | | | 170 | 133.2 | 55.6 | 12.9 |
| 10% CXB | 480 mb; hyd; | 5 mg | 14 | 310.9 | 811.1 | 489.9 |
| hydrogel | 27.8% cxbst; | | 86 | 253.8 | 1203.3 | 4026.7 |
| | 3% SFf; | | 127 | 8.3 | 47.2 | 22.9 |
| | 10% CXBf; | | 170 | 1.6 | 87.6 | 8.1 |
| | 10% P188f | | | | | |
| CXB rods | 480 mb; | 0.7 mg | 14 | 4.6 | 27.5 | 8.9 |
| | 0.5 mm; 40% st; | | 29 | 788.8 | 14.7 | 83.6 |
| | 100 mgsf; | | 58 | 1.7 | 21.1 | 99.5 |
| | 200 mgcxb; lyo; | | 86 | 2.0 | 52.2 | 4.0 |
| | 33.3% sf; | | 126 | 2.0 | 11.3 | 5.7 |
| | 66.7% cxb | | 169 | 3.0 | 15.6 | 10.6 |

The administration of the silk fibroin compositions resulted in in vivo concentrations of CXB consistently above the IC₅₀ of celecoxib with its target protein, COX-2 (40 nM or 15 ng/mL). The administration of either the silk fibroin hydrogels or the rods resulted in a higher intraocular concentration of CXB near the ocular area of administration (e.g. the half of the eye in which the rod was positioned). The intraocular concentrations of CXB remained greater than the IC₅₀ of CXB over the course of the experiment. The silk fibroin hydrogels sustained intraocular concentrations of CXB greater than the estimated EC₈₀ (1-3 μM or 381-1143 ng/mL) for the first 86 days. About 3 months after hydrogel administration, the intraocular CXB concentration lowers, but it remains above the IC₅₀ for CXB for the remainder of the study. The silk rods delivered a lower, more consistent concentration of CXB over time in comparison with the hydrogels.

Regardless of proximity of the formulation to the area of the eye or the amount of time since injection, the silk fibroin hydrogel or rod compositions resulted in CXB concentrations at least 1.7-fold greater than the IC₅₀ in the vitreous humor and at least 4-fold greater than the IC₅₀ in the retina/choroid over the first 86 days. Over the course of 169 or 170 days, the silk fibroin rod or hydrogel compositions resulted in CXB concentrations at least 1.6-fold greater than the IC₅₀ in the vitreous humor and at least 4-fold greater than the IC₅₀ in the retina/choroid.

Over the first 86 days, administration of the hydrogels resulted in a concentration at least 250-fold greater than the IC₅₀ of celecoxib in the vitreous humor without the implant, at least 800-fold greater than the IC₅₀ of celecoxib in the vitreous humor with the implant, and at least 480-fold greater than the IC₅₀ of celecoxib in the retina/choroid. Over 170 days, administration of the hydrogels resulted in a concentration at least 1.6-fold greater than the IC₅₀ of celecoxib in the vitreous humor without the implant, at least 47-fold greater than the IC₅₀ of celecoxib in the vitreous humor with the implant, and at least 8-fold greater than the IC₅₀ of celecoxib in the retina/choroid over the course of the experiment.

Over the first 86 days, administration of the rods resulted in a concentration at least 1.7-fold greater than the IC₅₀ of celecoxib in the vitreous humor without the implant, at least 14-fold greater than the IC₅₀ of celecoxib in the vitreous humor with the implant, and at least 4-fold greater than the IC₅₀ of celecoxib in the retina/choroid. Over 169 days, administration of the rods resulted in a concentration at least 1.7-fold greater than the IC₅₀ of celecoxib in the vitreous humor without the implant, at least 11-fold greater than the IC₅₀ of celecoxib in the vitreous humor with the implant, and at least 4-fold greater than the IC₅₀ of celecoxib in the retina/choroid.

Both the hydrogel and the rod could deliver CXB at or above the EC₈₀, concentration of compound needed to elicit 80% of a complete response. The EC₅₀ was estimated to be 1-3 μM for CXB in this system. Hydrogel administration resulted in intraocular concentrations of CXB above the EC₈₀ for the first 86 days, but the intraocular concentration of CXB was at or below the efficacious range after 86 days. Rod administration resulted in intraocular concentrations at or near the efficacious range in the vitreous humor with the formulation for the first 86 days. The hydrogel platform was able to deliver CXB at concentrations at least 3 times the EC₈₀ for less than or equal to 3 months in all the ocular tissues.

Both the rod and hydrogel formulations showed residence in the intraocular space for at least 6 months. The results indicated that silk-fibroin hydrogels and silk-fibroin rod implants were both well-tolerated formulation options that maintained steady-state delivery of CXB to ocular tissues for at least 3-6 months. Even with the major differences in CXB dose (5 mg in the hydrogel; 700 μg in the rod). CXB levels were maintained in the back of the eye above the IC₅₀ for CXB to COX-2 over the course of the study. This indicated that the concentrations were in an efficacious range.

### Example 19. Macromolecular Therapeutic Agent Storage and Stability by a Silk Composition

### Silk Fibroin Isolation and Hydrogel Formation

Silk yarn is degummed at 100° C. for 120 minutes in 0.02 M sodium carbonate aqueous solution to remove sericin. 30 g of cut silk yarn is boiled in 1 L of deionized (DI) water with 0.02 M sodium carbonate for 80 minutes under stirring. Then the yarn is transferred to a new boiling 0.02 M sodium carbonate aqueous solution and boiled for additional 40 minutes under stirring. The fibroin is then placed in DI water at 60-70° C. for 20 minutes under stirring, and then rinsed with clean DI water. This is repeated three times. The fibroin is then placed in clean DI water and stirred for 20 minutes, then rinsed with clean DI water and repeated for a total of three 20 minute-rinse cycles. The fibroin is then dried overnight, weighed, and dissolved at 20% (w/v) in a 9.3 M aqueous solution of lithium bromide for 5 hours at 60° C. The resulting fibroin solution is dialyzed against water at 4° C. in a 50 kDa regenerated cellulose dialysis tubing for 48 hours with 6 water changes to remove the excess salt. The conductivity is recorded after each water change with a digital quality tester. When the conductivity is under 5 ppm the fibroin is ready.

The solution is centrifuged three times for 20 minutes each at 9,000 RPM and 4° C. to remove insoluble particles. The supernatant is collected, and samples of the supernatant are diluted at 1:20 and 1:40 in water. Standard samples are prepared for an A280 assay by diluting pre-measures fibroin solutions to 5, 2.5, 1.25, 0.625, 0.3125, and 0 mg/mL in water, for the generation of a standard curve. The silk concentration of the 1:20 and 1:40 diluted silk fibroin samples is measured against the standard curve using absorbance at 280 nm.

The fibroin solutions are diluted to a final concentration of 3% (w/v) in 10 mM phosphate buffer or TRIS buffer, pH 7.4. Some solutions of silk fibroin are also prepared with 0.5-5% (w/v) sucrose and/or 2-10 mM histidine buffer. The solutions are filtered through a 0.2 μm filter using a vacuum filter unit. Sucrose can be added to the solution prior to freezing to aid in reconstitution of the lyophilized silk fibroin after lyophilization. Then, 10 mL of each solution is aliquoted into 50 mL conical tubes, snap frozen in liquid nitrogen for 10 minutes, transferred for 20 minutes in −80° C., and lyophilized for 72 hours.

### Therapeutic Agent Loading in Silk Fibroin Hydrogel

Lyophilized silk fibroin is dissolved with a solution of the therapeutic agent to obtain concentrations of 1.3, 3.6, 7.0, 13.0, and 23.0% (w/v) silk fibroin. A gelling agent (PEG400, glycerol, Poloxamer, etc.) is added to the therapeutic/silk solution to induce gel formation. The tube can be left at 4° C., room temperature (RT) or 37° C. overnight to induce gelation.

### Stability of Therapeutic Agent

The effect of silk fibroin hydrogel on the stability of the therapeutic agent is evaluated by placing samples of the therapeutic loaded silk fibroin hydrogel at different temperatures (4° C., 25° C. or 37° C.). At weekly timepoints, the therapeutic agent is extracted from the formulation by placing a known mass of the formulation into a compatible buffer. The extracted solution is analyzed by using a stability indicating HPLC assay as well as a cell-based activity assay. The structural integrity of the formulation and/or the therapeutic agent is determined by using an HPLC assay and evaluating the presence of aggregation. The functional activity of the therapeutic is evaluated by using a cell-based assay.

### In Vitro Release

An aliquot of the fibroin-therapeutic hydrogel is added to a 2-mL Eppendorf tube. 1.95 mL of release medium (PBS, pH 7.4) is added. The samples are incubated at 37° C. with gentle shaking. The release medium is changed after 24 hours and then approximately once daily for 7 days. The release medium is analyzed by HPLC to determine therapeutic concentration. A calibration curve is generated for the therapeutic agent by dissolving a known amount of the therapeutic agent in the release medium.

### Example 20. Macromolecular Therapeutic Agent Storage and Stability by Silk Fibroin Solutions

Lyophilized silk fibroin is dissolved in water to obtain concentrations of 1.3, 3.6, 7.0, 13.0, and 23.0% (w/v) silk fibroin. These silk fibroin solutions are used as stock solutions to prepare therapeutic solutions comprising 0.1%-30% silk fibroin and a therapeutic agent. The therapeutic solution is formulated with excipients and buffers including the silk fibroin solution.

The effect of the silk fibroin solutions on the stability of the therapeutic agent is evaluated by placing solutions of the therapeutic solutions containing silk fibroin at different temperatures (4° C., 25° C. or 37° C.). At weekly timepoints, the therapeutic solution is analyzed by using a stability indicating HPLC assay as well as a cell-based activity assay. The HPLC assay determines structural integrity of the formulation by evaluating the presence of aggregation. The functional activity of the therapeutic agent is evaluated by using a cell-based assay.

### Example 21. Macromolecular Therapeutic Agent Lyophilization Stability by Silk Fibroin

Lyophilized silk fibroin is dissolved in water to obtain concentrations of 1.3, 3.6, 7.0, 13.0, and 23.0% (w/v) silk fibroin. These silk fibroin solutions are used as stock solutions to prepare therapeutic solutions comprising 0.1%-30% silk fibroin and a therapeutic agent. The therapeutic agent is formulated with excipients and buffers including the silk fibroin solution. These solutions are then placed in glass vials, frozen and lyophilized.

The effect of silk fibroin solutions on the stability of the therapeutic agent through lyophilization is evaluated by placing the lyophilized vials of the therapeutic containing silk fibroin at different temperatures (4° C., 25° C. or 37° C.). At weekly timepoints, the therapeutic formulation is reconstituted. The reconstituted solution is analyzed by using a stability indicating HPLC assay as well as a cell-based activity assay. The HPLC assay determines the structural integrity of the formulation by evaluating the presence of aggregation. The functional activity of the therapeutic agent is evaluated by using a cell-based assay.

### Example 22. Release Characteristics of Celecoxib from Silk Fibroin Hydrogels of Varying

### Silk Fibroin Molecular Weights

Silk yarn was purchased from Jiangsu SOHO Silk and Textile Co. (Jiangsu, China). Lithium Bromide was purchased from Sigma-Aldrich (St. Louis, Mo.). Polysorbate-80 was purchased from Croda (Snaith, United Kingdom). The potassium phosphate monobasic and the potassium phosphate dibasic were purchased from Sigma Aldrich Fine Chemicals (St. Louis, Mo.). The glycerol, sodium carbonate, and sodium azide were purchased from Fisher Chemical (Waltham, Mass.). The celecoxib (CXB) was purchased from Cipla (Miami, Fla.).

### Silk Fibroin Isolation

Silk yarn from SOHO was degummed at 100° C. for either 30, 60, 90, 120, or 480 minutes in 0.02 M sodium carbonate solution to remove sericin and modify fibroin molecular weight. The amount of boiling time was referred to as the “minute boil” or “mb”. Longer boiling times produced silk fibroin with smaller molecular weights. 480 mb silk fibroin has an average molecular weight of between 30-60 kDa, 120 mb silk fibroin has an average molecular weight of between 100-300 kDa, and 90 mb silk fibroin has an average molecular weight of about 361 kDa. Fibroin was dried overnight, weighed, and dissolved at 20% (w/v) in 9.3 M lithium bromide solution for five hours at 60° C. The resulting solution was dialyzed against water in a 50 kDa regenerated cellulose membrane for 48 hours at 4° C. with six water changes. The resulting solution was centrifuged for 20 minutes at 9,000 RPM and 4° C. to remove insoluble particles. Solutions were diluted to a final concentration of 3% (w/v) in 10 mM phosphate buffer, pH 7.4, filtered through a 0.22 μm filter, frozen in liquid nitrogen, and lyophilized for at least 72 hours. Lyophilized silk fibroin was stored at −20° C. or less prior to use.

### Hydrogel Preparation

Lyophilized silk-fibroin was reconstituted to a concentration of 6% (w/v) using a suspension of celecoxib. The silk/CXB suspension had a final concentration of 6% (w/v) silk-fibroin, 20% (w/v) CXB in suspension, 0.2% polysorbate-80, and 44 mM phosphate buffer. Silk/CXB and 80% glycerol in water solutions were then combined at a ratio of 1:1 and mixed until homogeneous. The final formulation for all hydrogels prepared was: 3% (w/v) silk-fibroin, 40% glycerol, 10% CXB, 0.1% tween-80, and 22 mM phosphate buffer, pH7.4. Gels were incubated at 37° C. on an orbital mixer overnight to induce gelation, and the hydrogels were stored at 4° C. until use. The formulations tested were named by the method in which they were prepared. For example, in the sample named 480 mb; hyd; 3% SFf; 10% XBf; 40% Glyc, “480 mb” refers to silk degummed with a 480-minute boil, “hyd” refers to the formulation of the sample as a hydrogel, “3% SFf” refers to a formulation with 3% (w/v) silk fibroin, “10% CXBf” refers to a formulation with 10% (w/v) celecoxib, and “40% Glyc” refers to a formulation with 40% (w/v) glycerol. Some samples were prepared with silk fibroin degummed with a 120, 90, 60, or 30-minute boil (120 mb 90 mb, 60 mb, and 30 mb respectively). The formulations were listed in Table 49. In Table 49 “PS-80” is Polysorbate-80.

TABLE 49
Formulations of silk fibroin hydrogels prepared from silk fibroin degummed
with different boiling times for the cumulative release experiments

| | | | | | | | Actual | |
|---|---|---|---|---|---|---|---|---|
| | | | | | | | CXB | |
| | Silk | | | | | | conc. | |
| | boiling | Silk | TPS-80 | Phosphate | Glycerol | CXB | Standard | |
| Sample | time | conc. | conc. | Buffer | conc. | conc. | Deviation | Sample |
| name | (mb) | (% w/v) | (% w/v) | (mM) | (% w/v) | (% w/v) | (% w/v) | No. |
| 480 mb; hyd; | 480 | 3 | 0.1 | 22 | 40 | 10 | 10.92 ± 0.31 | 161-1 |
| 3% SFf; | | | | | | | | |
| 10% CXBf; | | | | | | | | |
| 40% Glycf | | | | | | | | |
| 120 mb; hyd; | 120 | 3 | 0.1 | 22 | 40 | 10 | 9.78 ± 0.22 | 161-2 |
| 3% SFf; | | | | | | | | |
| 1.0% CXBf; | | | | | | | | |
| 40% Glycf | | | | | | | | |
| 90 mb; hyd; | 90 | 3 | 0.1 | 22 | 40 | 10 | 9.27 ± 1.72 | 161-3 |
| 3% SFf; | | | | | | | | |
| 10% CXBf; | | | | | | | | |
| 40%; Glycf | | | | | | | | |
| 60 mb; hyd; | 60 | 3 | 0.1 | 22 | 40 | 10 | 9.19 ± 0.52 | 161-4 |
| 3% SFf; | | | | | | | | |
| 10% CXBf; | | | | | | | | |
| 40% Glycf | | | | | | | | |
| 30 mb; hyd; | 30 | 3 | 0.1 | 22 | 40 | 10 | 9.34 ± 0.78 | 161-5 |
| 3% SFf; | | | | | | | | |
| 10% CXBf; | | | | | | | | |
| 40% Glycf | | | | | | | | |
| Solution | N/A | 0 | 0.1 | 22 | 40 | 10 | 11.68 ± 0.67 | 161-6 |
| control | | | | | | | | |

### In Vitro Release of Celecoxib

In triplicate, 50 mg of each formulation was weighed into half of a #4 gelatin capsule. Capsules were placed into a 50 mL. conical tube containing 45 mL of release medium (1× phosphate buffered saline, 2% Polysorbate-80, and 0.05 sodium azide). The solubility of celecoxib in this release media is 850 μg/mL. 45 mL of this release media allows for 38 mg CXB solubility. This media ensured sink conditions (greater than or equal to 5 times CXB solubility) were maintained throughout the course of the study. The tubes were capped and incubated at 37° C. with shaking. 1 mL of the release media was collected from each sample at days 1, 4, 7, 10, 14 and 21 days and replaced with fresh media. At each timepoint, the tubes were stood on end for at least 30 minutes. to allow the formulation to settle prior to taking the sample. Release media was analyzed by HPLC-UV (Agilent 1290 Infinity) at 260 nm. Controls were prepared at Day 0 by weighing 50 mg of each formulation in triplicate in separate 20 mL. glass vials. Methanol was added to each sample to extract CXB. Samples were placed on a shaker at room temperature for 24 hrs. The supernatant was analyzed by HPLC-UV to determine CXB loading. The results of the release studies were displayed in Table 50A and Table 50B.

TABLE 50A
In vitro release kinetics for silk fibroin hydrogels with varying molecular weight silk loaded
with celecoxib; average cumulative percentage (%) of API released

| | Day | | | | | | |
|---|---|---|---|---|---|---|---|
| Sample No. | 0 | 1 | 3 | 7 | 14 | 21 | 28 |
| 161-1 | 0.0 | 69.6 | 93.0 | 89.1 | 91.6 | — | — |
| 161-2 | 0.0 | 26.9 | 49.0 | 66.8 | 82.2 | 91.9 | 86.4 |
| 161-3 | 0.0 | 28.9 | 54.6 | 74.2 | 90.1 | 100.3 | 94.1 |
| 161-4 | 0.0 | 27.5 | 51.3 | 67.0 | 81.6 | 89.0 | 83.8 |
| 161-5 | 0.0 | 47.7 | 71.7 | 78.1 | 86.3 | 91.7 | 86.4 |
| 161-6 | 0.0 | 70.9 | 95.1 | 93.7 | 93.0 | — | — |

TABLE 50B
Standard Deviation of in vitro release kinetics for silk fibroin
hydrogels with varying molecular weight silk loaded with celecoxib;
in terms of average cumulative percentage (%) of API released

| Sample | Day | | | | | | |
|---|---|---|---|---|---|---|---|
| No. | 0 | 1 | 3 | 7 | 14 | 21 | 28 |
| 161-1 | 0.0 | 7.2 | 1.8 | 3.3 | 7.0 | — | — |
| 161-2 | 0.0 | 2.2 | 1.2 | 2.0 | 1.3 | 2.8 | 2.6 |
| 161-3 | 0.0 | 9.0 | 0.5 | 3.3 | 4.5 | 5.7 | 5.5 |
| 161-4 | 0.0 | 1.8 | 2.6 | 3.9 | 5.1 | 6.1 | 5.8 |
| 161-5 | 0.0 | 21.9 | 12.1 | 6.0 | 4.6 | 6.5 | 6.2 |
| 161-6 | 0.0 | 5.6 | 4.6 | 4.3 | 5.5 | — | — |

The 480 mb hydrogels approached 100% CXB release the quickest following a similar trajectory to the CXB suspension alone. This was most likely due to the formulation not completely gelling. When placed in release media it did not hold its shape and it dispersed as a suspension. Formulations prepared with the higher molecular weight range of silk-fibroin displayed similar release profiles following first-order release kinetics, with an initial burst of approximately 30% out to 21 days, with the exception of the hydrogel made with highest silk-fibroin molecular weight (30 mb). This formulation displayed a slightly higher burst than the others, but the release continued out to 21 days.

### Example 23. Rheological Characteristics of Celecoxib-containing Silk-Fibroin Hydrogels of Varying Silk Fibroin Molecular Weights

Silk yarn was purchased from Jiangsu SOHO Silk and Textile Co. (Jiangsu, China). Lithium Bromide was purchased from Sigma-Aldrich (St. Louis, Mo.). Polysorbate-80 was purchased from Croda (Snaith, United Kingdom). The potassium phosphate monobasic and the potassium phosphate dibasic were purchased from Sigma Aldrich Fine Chemicals (St. Louis, Mo.). The glycerol, sodium carbonate, and sodium azide were purchased from Fisher Chemical (Waltham, Mass.). The celecoxib (CXB) was purchased from Cipla (Miami, Fla.).

### Silk Fibroin Isolation

Silk yarn from SOHO was degummed at 100° C. for either 30, 60, 90, 120, or 480 minutes in 0.02 M sodium carbonate solution to remove sericin and modify fibroin molecular weight. The amount of boiling time was referred to as the “minute boil” or “mb”. Longer boiling times produced silk fibroin with smaller molecular weights. 480 mb silk fibroin has an average molecular weight of between 30-60 kDa, 120 mb silk fibroin has an average molecular weight of between 100-300 kDa, and 90 mb silk fibroin has an average molecular weight of about 361 kDa. Fibroin was dried overnight, weighed, and dissolved at 20% (w/v) in 9.3 M lithium bromide solution for five hours at 60° C. The resulting solution was dialyzed against water in a 50 kDa regenerated cellulose membrane for 48 hours at 4° C. with six water changes. The resulting solution was centrifuged for 20 minutes at 9,000 RPM and 4° C. to remove insoluble particles. Solutions were diluted to a final concentration of 3% (w/v) in 10 mM phosphate buffer, pH 7.4, filtered through a 0.22 μm filter, frozen in liquid nitrogen, and lyophilized for at least 72 hours. Lyophilized silk fibroin was stored at −20° C. or less prior to use.

### Hydrogel Preparation

Lyophilized silk-fibroin was reconstituted to a concentration of 6% (w/v) using a suspension of celecoxib. The silk/CXB suspension had a final concentration of 6% (w/v) silk-fibroin, 20% (w/v) CXB in suspension, 0.2% polysorbate-80, and 44 mM phosphate buffer. Silk/CXB and 80% glycerol in water solutions were then combined at a ratio of 1:1 and mixed until homogeneous. The final formulation for all hydrogels prepared was: 3% (w/v) silk-fibroin, 40% glycerol, 10% CXB, 0.1% polysorbate-80, and 22 mM phosphate buffer, pH 7.4. Gels were incubated at 37° C. on an orbital mixer overnight to induce gelation, and the hydrogels were stored at 4° C. until use. The formulations tested were named by the method in which they were prepared. For example, in the sample named 480 mb; hyd; 3% SFf; 10% CXBf; 40% Glyc, “480 mb” refers to silk degummed with a 480-minute boil, “hyd” refers to the formulation of the sample as a hydrogel, “3% SFf” refers to a formulation with 3% (w/v) silk fibroin, “10% CXBf” refers to a formulation with 10% (w/v) celecoxib, and “40% Glyc” refers to a formulation with 40% (w/v) glycerol. Some samples were prepared with silk fibroin degummed with a 120, 90, 60, or 30-minute boil (120 mb, 90 mb, 60 mb, and 30 mb respectively). The formulations were listed in Table 51.

TABLE 51
Formulations of silk fibroin hydrogels prepared from silk fibroin degummed
with different boiling times for the theological experiments

| | Silk | | | | | |
|---|---|---|---|---|---|---|
| | boiling | Silk | Polysorbate- | Phosphate | Glycerol | CXB |
| | time | conc. | 80 conc. | Buffer | conc. | conc. |
| Sample name | (mb) | (% w/v) | (% w/v) | (mM) | (% w/v) | (% w/v) |
| 480 mb; hyd; 3% SFf; | 480 | 3 | 0.1 | 22 | 40 | 10 |
| 10% CXBf; 40% Glyc | | | | | | |
| 120 mb; hyd; 3% SFf; | 120 | 3 | 0.1 | 22 | 40 | 10 |
| 10% CXBf; 40% Glyc | | | | | | |
| 90 mb; hyd; 3% SFf; | 90 | 3 | 0.1 | 22 | 40 | 10 |
| 10% CXBf; 40% Glyc | | | | | | |
| 60 mb; hyd; 3% SFf; | 60 | 3 | 0.1 | 22 | 40 | 10 |
| 10% CXBf; 40% Glyc | | | | | | |
| 30 mb; hyd; 3 SFf; | 30 | 3 | 0.1 | 22 | 40 | 10 |
| 10% CXBf; 40% Glyc | | | | | | |

### Rheological Measurements of Silk Fibroin Formulations

The hydrogel samples were loaded onto a Peltier plate system held at 25° C. The geometry used was a 20 mm parallel plate with a gap of 1 mm and frequency at 1 Hz. Viscosity was measured during a time sweep at 1 s-1 over 135 seconds. The storage modulus (G′), the loss modulus (G″), and the phase angle were then measured during a time sweep over 145 seconds at 0.1% strain and 1 Hz. As seen in Table 52, the rheology showed a general increase in viscosity from silk fibroin prepared from a longer boiling time (480 mb) to silk fibroin prepared from a shorter boiling time (30 mb); therefore, the viscosity increased from low molecular weight silk-fibroin to high molecular weight silk-fibroin formulations. In Table 52, “Std. Dev.” refers to standard deviation.

TABLE 52
Rheological properties of silk fibroin hydrogels with celecoxib.

| | | | | | Phase | | | | |
|---|---|---|---|---|---|---|---|---|---|
| | | | Viscosity | | Angle | | | | |
| | Boil | | Std. | Phase | Std. | | G′ Std. | | G″ Std. |
| Sample | Time | Viscosity | Dev. | Angle | Dev. | G′ | Dev. | G″ | Dev. |
| No. | (mb) | (Pa*s) | (Pa*s) | (°) | (°) | (Pa) | (Pa) | (Pa) | (Pa) |
| P00161-01 | 480 | 6.56 | 1.67 | 9.60 | 2.97 | 76.98 | 8.43 | 12.91 | 3.85 |
| P00161-02 | 120 | 49.72 | 2.81 | 8.43 | 0.43 | 1148.30 | 93.06 | 169.69 | 8.63 |
| P00161-03 | 90 | 65.25 | 2.25 | 8.88 | 0.48 | 1652.94 | 134.85 | 257.58 | 13.09 |
| P00161-04 | 60 | 118.64 | 6.55 | 12.41 | 0.68 | 4279.45 | 276.60 | 939.41 | 45.63 |
| P00161-05 | 30 | 169.61 | 7.40 | 14.78 | 1.22 | 7820.86 | 539.69 | 2057.36 | 145.74 |

The viscosities ranged from 7 to 170 Pa s-1 for the range of molecular weights tested. PG The stiffness (as measured by G′ and G″, seen in Table 52) also showed an increase with increasing molecular weight of silk-fibroin, as defined by the minute boil. The phase angle, as seen in Table 52, increased slightly for the hydrogel formulations prepared from silk fibroin with a shorter boiling time. As the molecular weight of the silk-fibroin increased (marked by a lower degumming time) the hydrogel formulations were stiffer and much more viscous. These results displayed the range of properties the silk-fibroin hydrogel formulations could have. The formulations had also been used to analyze the release of CXB over time, and the physical characteristics of the hydrogels were able to be modified while only minimally affecting release kinetics.

### Example 24. Rheology Studies of Silk Fibroin Hydrogels

Hydrogel samples were loaded into a Peltier plate system, with a 20-mm parallel plate geometry, at a temperature of 25° C. The gap was set to 1 mm, and the frequency was set to 1 Hz. Viscosity measurements were measured with a shear ramp was from 0.1 1/s to 1 1/s over 113 s with 11 samples, followed by a shear hold at 1 1/s for 180s with 18 samples. Oscillatory measurements were measured with a strain ramp from 0.01 to 1% strain with a constant 1 Hz frequency over 173s with 21 measurements and the G′, G″, and phase angle were averaged over the linear viscoelastic region (LVR). The viscosity was first studied as a function of silk fibroin concentration, as seen in Table 53. The viscosity of the silk fibroin hydrogels was studied for hydrogels with two different excipients. Silk fibroin hydrogels were studied with silk fibroin concentrations of 6%, 5%, 4%, 3%, and 2% (w/v) silk fibroin degummed with a 120-minute boil. The hydrogels were prepared with either 40% PEG300 or 40% glycerol, 0.2% polysorbate-80, 22 mM phosphate buffer, and 10% celecoxib (CXB). The components of the gel were mixed and allowed to gel at 37° C. with rotation.

TABLE 53
Rheological properties of silk fibroin hydrogels with varying concentrations of silk fibroin

| | | | | | | | | Standard Deviation of the | | | |
|---|---|---|---|---|---|---|---|---|---|---|---|
| | | | | Average | | | | Average | | | |
| | Silk | | | | | Phase | | | | Phase | |
| Sample | Fibroin | | Excip. | G′ | G″ | Angle | Visc. | G′ | G″ | Angle | Visc. |
| No. | % | Excip. | % | (Pa) | (Pa) | (°) | (Pa*s) | (Pa) | (Pa) | (°) | (Pa*s) |
| 130-01 | 6 | PEG300 | 40 | 71188 | 12346 | 10.13 | 1798 | 30242 | 4469 | 0.53 | 687 |
| 130-02 | 6 | Glycerol | 40 | 80647 | 12307 | 8.77 | 1722 | 46411 | 6745 | 0.39 | 957 |
| 130-03 | 5 | PEG300 | 40 | 33297 | 5859 | 10.04 | 717 | 8723 | 1426 | 0.27 | 184 |
| 130-04 | 5 | Glycerol | 40 | 33737 | 5054 | 8.54 | 726 | 12631 | 1873 | 0.16 | 275 |
| 130-05 | 4 | PEG300 | 40 | 21504 | 3845 | 10.24 | 364 | 8124 | 1409 | 0.48 | 142 |
| 130-06 | 4 | Glycerol | 40 | 18618 | 2677 | 8.21 | 379 | 6331 | 886 | 0.11 | 111 |
| 130-07 | 3 | PEG300 | 40 | 4968 | 996 | 11.52 | 57 | 440 | 101 | 2.12 | 1 |
| 130-08 | 3 | Glycerol | 40 | 7511 | 1046 | 7.95 | 161 | 2977 | 410 | 0.15 | 68 |
| 130-09 | 2 | PEG300 | 40 | 2484 | 473 | 11.05 | 34 | 1923 | 365 | 1.29 | 26 |
| 130-10 | 2 | Glycerol | 40 | 1814 | 257 | 8.24 | 31 | 1915 | 264 | 0.27 | 18 |

The viscosity of the hydrogels increased with the concentration of silk fibroin.

### Example 25. Formulation and Release Characteristic of Rods of Increased Hydrophilicity

SBPs were formulated as rods to determine whether soluble and/or bulky additives to silk fibroin rod formulations would increase API release. These additives were also included to enhance and increase the rate of in vivo degradation of silk fibroin rods. The silk fibroin was degummed for 480 minutes. The formulations tested were named by the method in which they were prepared. For example, in the sample named “480 mb; 0.5 mm; 20% st; 50mgsf; 200mgcxb; oven; 14.8% sf: 59.3% cxb; 25.9% sucrose/poly-20” refers to a silk fibroin rod prepared from silk degummed with a 480-minute boil (480 mb), an extrusion with a 0.5 mm diameter (0.5 mm), a preparation from a 20% stock solution of silk fibroin (20% st), a preparation from 50 mg of silk fibroin (50mgsf), a preparation from 200 mg of celecoxib (200mgcxb), oven drying (oven), a theoretical w/v percentage of 14.8% silk fibroin (14.8% sf), a theoretical w/v percentage of 59.3% celecoxib (59.3% cxb), and a theoretical w/v percentage of 25.9% other additives such as sucrose and polysorbate-20 (25.9% sucrose/poly-20). The samples tested were listed in Table 54. Other additives tested included polysorbate-80 (poly-80), trehalose, mannitol, PEG 2 kDa, hydroxyethylcellulose (HEC), carboxymethylcellulose (CMC), polyvinylpyrrolidone K-17 (K17), and polyvinylalcohol (PVA). The term theoretical loading percentage refers to the assumed percentage of a component incorporated in a substance or product. The product may be an SBP.

TABLE 54
Formulations of silk fibroin hydrogels prepared with various fillers to alter hydrophilicity

| | | | Theoretical | Theoretical | Theoretical |
|---|---|---|---|---|---|
| Sample | Formulation | | dry CXB | dry SF | dry other |
| number | Description | Name | (mg) | (mg) | (mg) |
| 222-01 | 40% SF; | 480 mb; 0.5 mm; | 200 | 100 | 0 |
| | Control; | 40% st; 100 mgsf; | | | |
| | Oven Dried | 200 mgcxb; oven; | | | |
| | | 33.3% sf; 66.7% cxb | | | |
| 222-03 | 20% SF; 70% | 480 mb; 0.5 mm; | 200 | 50 | 87.5 |
| | Sucrose + 0.5% | 20% st; 50 mgsf; | | | |
| | Polysorbate-20; | 200 mgcxb; oven; | | | |
| | Oven Dried | 14.8% sf; 59.3% | | | |
| | | cxb; 25.9% sucrose/ | | | |
| | | poly-20 | | | |
| 222-05 | 20% SF; 70% | 480 mb; 0.5 mm; | 200 | 50 | 87.5 |
| | Sucrose + 0.5% | 20% st; 50 mgsf; | | | |
| | Polysorbate-80; | 200 mgcxb; oven; | | | |
| | Oven Dried | 14.8% sf; 59.3% | | | |
| | | cxb; 25.9% sucrose/ | | | |
| | | poly-80 | | | |
| 222-09 | 20% SF; 70% | 480 mb; 0.5 mm; | 200 | 50 | 87.5 |
| | Trehalose; | 20% st; 50 mgsf; | | | |
| | Oven Dried | 200 mgcxb; oven; | | | |
| | | 14.8% sf; 59.3% | | | |
| | | cxb; 25.9% trehalose | | | |
| 222-11 | 20% SF; 70% | 480 mb; 0.5 mm; | 200 | 50 | 87.5 |
| | Trehalose + 0.5% | 20% st; 50 mgsf, | | | |
| | Polysorbate-80; | 200 mgcxb; oven, | | | |
| | Oven Dried | 14.8% sf; 59.3% | | | |
| | | cxb; 25.9% trehalose/ | | | |
| | | poly-80 | | | |
| 222-15 | 20% SF; 70% | 480 mb; 0.5 mm; | 200 | 50 | 87.5 |
| | Mannitol + 0.5% | 20% st; 50 mgsf; | | | |
| | Polysorbate-80; | 200 mgcxb; oven; | | | |
| | Oven Dried | 14.8% sf; 59.3% | | | |
| | | cxb; 25.9% mannitol/ | | | |
| | | poly-80 | | | |
| 222-17 | 20% SF; 50% | 480 mb; 0.5 mm; | 200 | 50 | 62.5 |
| | PEG 2 kDa; | 20% st; 50 mgsf; | | | |
| | Oven Dried | 200 mgcxb; oven; | | | |
| | | 50% 16.0% sf; | | | |
| | | 64.0% cxb; 20.0% | | | |
| | | PEG2 kDa | | | |
| 222-19 | 20% SF; 5% | 480 mb; 0.5 mm; | 200 | 50 | 6.25 |
| | HEC + 0.05% | 20% st; 50 mgsf; | | | |
| | Polysorbate-20; | 200 mgcxb; oven; | | | |
| | Oven Dried | 19.5% sf; 78.0% | | | |
| | | cxb; 2.4% hec/ | | | |
| | | poly-20 | | | |
| 222-21 | 20% SF; 5% | 480 mb; 0.5 mm; | 200 | 50 | 6.25 |
| | CMC + 0.05% | 20% st; 50 mgsf; | | | |
| | Polysorbate-20; | 200 mgcxb; oven; | | | |
| | Oven Dried | 19.5% sf; 78.0% | | | |
| | | cxb; 2.4% cmc/ | | | |
| | | poly-20 | | | |
| 222-25 | 20% SF; 20% | 480 mb; 0.5 mm; | 200 | 50 | 25 |
| | K17 + 0.05% | 20% st; 50 mgsf, | | | |
| | Polysorbate-20; | 200 mgcxb; oven, | | | |
| | Oven Dried | 18.2% sf; 72.7% | | | |
| | | cxb; 9.1% k17/ | | | |
| | | poly-20 | | | |
| 222-27 | 20% SF; 5% | 480 mb; 0.5 mm; | 200 | 50 | 6.25 |
| | PVA + 0.05% | 20% st; 50 mgsf; | | | |
| | Polysoibate-20; | 200 mgcxb; oven; | | | |
| | Oven Dried | 19.5% sf; 78.0% | | | |
| | | cxb; 2.4% pva/ | | | |
| | | poly-20 | | | |

The density of the experimental loadings as well as the densities of the silk fibroin rods were also determined, as seen in Table 55. The differences in theoretical and experimental loadings of celecoxib were also determined as a percentage of the theoretical w/w loading of celecoxib. In Table 55, “Std. Dev.” refers to standard deviation.

TABLE 55
Experimental loadings and densities of silk fibroin rods with increased hydrophilicity

| | | | | % Difference | | |
|---|---|---|---|---|---|---|
| | | | | between | | |
| | | | Std. Dev. | theoretical and | Std. | |
| Sample | Expermental % | Experimental % | of exp. % | actual loading | Density | Dev. of |
| number | SF | CXB | CXB | of CXB | (g/mL) | Density |
| 222-01 | 36.44 | 63.56 | 2.83 | −5% | 1.09 | 0.03 |
| 222-03 | 38.07 | 61.93 | 1.16 | 5% | 1.03 | 0.04 |
| 222-05 | 36.58 | 63.42 | 3.60 | 7% | 0.96 | 0.07 |
| 222-09 | 45.41 | 54.59 | 3.43 | −8% | 1.06 | 0.04 |
| 222-11 | 36.95 | 63.05 | 1.19 | 6% | 1.11 | 0.06 |
| 222-15 | 26.57 | 73.43 | 1.64 | 24% | 0.92 | 0.01 |
| 222-17 | 39.10 | 60.90 | 2.44 | −5% | 1.13 | 0.08 |
| 222-19 | 19.87 | 80.13 | 3.69 | 3% | 0.82 | 0.04 |
| 222-21 | 20.24 | 79.76 | 5.44 | 2% | 0.85 | 0.06 |
| 222-25 | 19.51 | 80.49 | 3.84 | 11% | 0.87 | 0.07 |
| 222-27 | 20.26 | 79.74 | 3.88 | 2% | 0.86 | 0.01 |

The silk fibroin rods were subject to in vitro release experiments to determine the release kinetics of celecoxib from these formulations. The silk fibroin rods were incubated in PBS with 0.6% polysorbate-80 and 0.05% sodium azide over the course of the experiment. The average cumulative release percentage of celecoxib over time was depicted in the release kinetics shown in Table 56A and Table 56B.

TABLE 56A
In vitro release kinetics for hydrophilic silk fibroin rods loaded with celecoxib;
average cumulative percentage (%) of API released

| | Sample Number | | | | | | | | | | | |
|---|---|---|---|---|---|---|---|---|---|---|---|---|
| | 222- | 222- | 222- | 222- | 222- | 222- | 222- | 222- | 222- | 222- | 222- | CXB |
| Day | 01 | 03 | 05 | 09 | 11 | 15 | 17 | 19 | 21 | 25 | 27 | suspension |
| 0 | 0.0 | 0.0 | 0.0 | 0.0 | 0.0 | 0.0 | 0.0 | 0.0 | 0.0 | 0.0 | 0.0 | 0.0 |
| 1 | 12.7 | 18.7 | 17.1 | 21.4 | 18.1 | 16.1 | 20.1 | 21.2 | 16.4 | 18.6 | 20.5 | 106.8 |
| 4 | 31.9 | 44.4 | 47.7 | 50.7 | 44.6 | 45.6 | 48.3 | 55.4 | 44.4 | 53.0 | 49.5 | 110.1 |
| 7 | 46.3 | 60.8 | 63.0 | 64.4 | 60.3 | 61.8 | 67.3 | 71.4 | 57.7 | 67.7 | 60.4 | 91.6 |
| 14 | 55.0 | 70.2 | 74.5 | 77.0 | 72.7 | 75.3 | 80.1 | 82.9 | 70.7 | 82.6 | 74.8 | 87.7 |
| 21 | 63.3 | 78.6 | 92.0 | 86.7 | 82.5 | 85.2 | 92.2 | 81.7 | 85.2 | 85.0 | 84.7 | 86.5 |
| 28 | 81.5 | 93.5 | 91.7 | 94.5 | 99.2 | 99.0 | 100.9 | 96.3 | 97.6 | 98.8 | 93.7 | 90.4 |
| 35 | 83.5 | 91.4 | 88.8 | 91.2 | 94.7 | 94.4 | 97.1 | 88.5 | 92.1 | 89.1 | 89.3 | 83.3 |
| 42 | 88.6 | 90.3 | 88.7 | 91.4 | 92.5 | 95.0 | 97.2 | 89.0 | 92.0 | 92.4 | 87.6 | — |
| 49 | 92.1 | 91.3 | 89.2 | 92.5 | 95.0 | 95.1 | 99.3 | 88.9 | 92.7 | 87.8 | 87.7 | — |
| 56 | 93.6 | 92.2 | 89.8 | 93.9 | 96.4 | 96.6 | 99.1 | 90.6 | 93.5 | 94.7 | 89.7 | — |

TABLE 56B
Standard deviation of average cumulative percentage of API released
in vitro for hydrophilic silk fibroin rods loaded with celecoxib

| | Sample Number | | | | | | | | | | | |
|---|---|---|---|---|---|---|---|---|---|---|---|---|
| | 222- | 222- | 222- | 222- | 222- | 222- | 222- | 222- | 222- | 222- | 222- | CXB |
| Day | 01 | 03 | 05 | 09 | 11 | 15 | 17 | 19 | 21 | 25 | 27 | suspension |
| 0 | 0.0 | 0.0 | 0.0 | 0.0 | 0.0 | 0.0 | 0.0 | 0.0 | 0.0 | 0.0 | 0.0 | 0.0 |
| 1 | 0.9 | 0.7 | 0.4 | 2.7 | 1.6 | 0.7 | 1.6 | 1.8 | 1.0 | 0.5 | 3.6 | 1.0 |
| 4 | 2.6 | 1.3 | 3.0 | 3.6 | 4.3 | 1.9 | 4.0 | 2.8 | 2.6 | 1.9 | 2.4 | 1.4 |
| 7 | 5.5 | 3.2 | 1.3 | 3.5 | 7.1 | 0.8 | 4.8 | 4.8 | 4.8 | 4.6 | 4.0 | 6.5 |
| 14 | 3.0 | 2.3 | 1.5 | 1.4 | 8.1 | 2.2 | 3.3 | 5.7 | 3.4 | 3.5 | 1.3 | 3.7 |
| 21 | 2.9 | 2.5 | 18.4 | 6.1 | 10.7 | 0.8 | 2.1 | 6.7 | 9.1 | 1.5 | 5.2 | 14.6 |
| 28 | 3.5 | 1.2 | 2.6 | 0.2 | 10.4 | 5.7 | 5.8 | 3.2 | 3.5 | 3.1 | 2.8 | 15.5 |
| 35 | 2.4 | 2.4 | 1.8 | 0.8 | 9.7 | 4.5 | 5.6 | 7.5 | 3.7 | 6.1 | 0.2 | 16.9 |
| 42 | 3.8 | 1.8 | 2.4 | 0.0 | 14.2 | 4.3 | 6.5 | 7.4 | 3.5 | 2.3 | 2.8 | — |
| 49 | 3.9 | 2.3 | 1.6 | 0.5 | 9.5 | 4.7 | 6.3 | 7.8 | 2.5 | 8.7 | 3.3 | — |
| 56 | 2.9 | 2.3 | 1.7 | 0.2 | 10.8 | 4.8 | 6.2 | 7.8 | 3.1 | 2.3 | 2.6 | — |

Overall, formulations with additives, including sucrose, trehalose, mannitol polysorbate-20, polysorbate-80, PEG 2 kDa, HEC, K17, CME, and PVA, showed increased API release during the initial burst as compared to silk fibroin rods without the additives. As used herein, the term “initial burst” refers to a rate of factor release from a source or depot over an initial release period (e.g., after administration or other placement, for example in solution during experimental analysis) that is higher than rates during one or more subsequent release periods. The initial burst was evaluated at 1 day for the silk fibroin rods. The silk fibroin rods with additives also demonstrated increased API release over the first 35 days of the experiment as compared to silk fibroin rods without the additives. These data suggested that additives to silk fibroin rods can be used to tune API release kinetics. The additives might also assist in rod degradation in vivo. The faster the drug is released from the formulation, the faster the majority of the surface area is exposed to the environment, and theoretically the faster the silk fibroin will degrade from enzymatic degradation. The control rod takes more time to disperse all of the API, and therefore will be around longer than rods that disperse API in less time.

### Example 26. Analysis of Celecoxib Remaining in Silk Fibroin Rods after In Vivo Administration

After the in vivo silk rods experiments, the silk fibroin rods were analyzed for the amount of celecoxib (CXB) that remained. At the desired timepoints of the in vivo experiments, New Zealand white rabbits were sacrificed, and their eyes were enucleated, snap frozen, and bisected. The formulation, hydrogel or implant (480 mb: 0.5 mm: 40% st; 100mgsf; 200mgcxb; lyo; 33.3% sf; 66.7% cxb) was removed from the eyes and collected for further studies. The vitreous containing the formulation was centrifuged at 10,000×g for 10 minutes. The resulting formulation pellet was frozen and lyophilized. Any remaining celecoxib was extracted from the formulations using acetonitrile and analyzed via HPLC-UV. Briefly, the formulation pellets were brought up in acetonitrile, and then vortexed, sonicated, and left on a shaker at room temperature for 24 to 48 hours. The supernatant was filtered through a 0.2 μm nylon syringe filter, diluted and then analyzed via HPLC-UV. The percentage of celecoxib remaining in the rod was studied as a function of time of in vivo study, as seen in Table 57.

TABLE 57
Celecoxib remaining in CXB loaded silk
rods after intravitreal injection

| | | | Average % | St. Dev. |
|---|---|---|---|---|
| Sample | Sample Name | Day | Remaining | (%) |
| Low CXB | 1.4% CXB | 14 | 65.0 | 19.9 |
| control | Suspension | 29 | 31.3 | 31.6 |
| | | 86 | 53.9 | 68.6 |
| High CXB | 10% CXB | 14 | 0.4 | 0.6 |
| control | Suspension | 86 | 88.8 | 3.6 |
| | | 127 | 139.2 | 39.6 |
| | | 170 | 87.0 | 0.6 |
| 10% CXB | 480 mb; hyd; | 14 | 100.3 | 20.2 |
| hydrogel | 27.8% cxbst; 3% | 86 | 49.9 | 17.2 |
| | SFf; 10% CXBf; | 127 | 87.0 | 22.5 |
| | 10% P188f | 170 | 81.6 | 7.9 |
| CXB rods | 480 mb; 0.5 mm; | 14 | 72.5 | 2.0 |
| | 40% st; 100 | 29 | 45.3 | 7.1 |
| | mgsf; 200 mgcxb; | 58 | 75.1 | 3.0 |
| | lyo; 33.3% sf; | 86 | 53.6 | 22.1 |
| | 66.7% cxb | 126 | 52.1 | 5.9 |
| | | 169 | 10.0 | 4.0 |

The concentration of celecoxib remaining in the rods decreased linearly as time passed. Extractions performed on the silk fibroin rods over the course of the study displayed a zero-order release of celecoxib in the vitreous. Fitting a curve to this linear regression demonstrated a good fit with the exception of the 1 month timepoint. The data demonstrated that approximately 10% of the loaded CXB still remained in the implant at 6 months. The in vivo half-life of release of the CXB from the rod implant, which represented the amount of time required for 50% of the celecoxib to be released from the silk fibroin rod, was estimated to be 3.5 months (about 85 days), with 90% CXB released by 6 months (169 days). Recoveries of the API from the hydrogel showed that there was still significant API remaining after completion of the study. The extractions of CXB from the rods and the hydrogels demonstrated that there was still sufficient CXB remaining to maintain steady-state delivery for at least 6 months with a single administration, since more than 50% of the celecoxib remained after 3 months of the experiment. Furthermore, the silk fibroin rods released CXB at a rate faster than that of the silk fibroin hydrogels in vivo.

### Example 27. Histopathology Studies of Rabbit Eyes with Silk Rods Compared with Silk Hydrogels

Eight formalin-fixed rabbit eyes were submitted to HistoTox Labs and processed into two blocks per sample. Eyes with gel formulations were collected at 203 days, and eyes with rod formulations were collected at 117 days. One slide per block was sectioned and stained with hematoxylin and eosin (H&E). Glass slides were evaluated by a board-certified veterinary pathologist using light microscopy. The presence of injected material was recorded, and histologic lesions were graded for severity (0=absent; 1=minimal; 2=mild; 3=moderate; 4=marked; 5=severe). The results of the experiment were summarized in Table 58. In Table 58, “P” refers to present and “NP” refers to not present.

TABLE 58
H&E grades of the rabbit eye histopathology data of animals
treated with silk fibroin rod and hydrogel compositions

| | | | H&E | | |
|---|---|---|---|---|---|
| | | | | Infiltration, | |
| | | | Injected | mononuclear cell/ | Retinal |
| | | | material, | multinucleated | distortion/ |
| | | | vitreous | cell, injected | degeneration, |
| Treatment | Sample | Block | chamber | material | focal |
| Silk-Fibroin | CCN-43L | 1 | NP | — | 0 |
| Hydrogel | | 2 | P (acellular | 0 | 0 |
| | | | aggregate) | | |
| | CCN-44L | 1 | NP | — | 0 |
| | | 2 | NP | — | 0 |
| | CCN-45L | 1 | NP | — | 0 |
| | | 2 | P (acellular | 0 | 0 |
| | | | aggregate) | | |
| Silk-Fibroin/ | CCN-86L | 1 | NP | — | 0 |
| CXB Rod | | 2 | P (rod) | 1 | 2 |
| | | 1 | P (rod) | 0 | 0 |
| | CCN-87L | 2 | P (rod) | 0 | 2 |
| | CCN-88L | 1 | NP | — | 0 |
| | | 2 | NP | — | 0 |
| Untreated | CCN-43R | 1 | NP | — | 0 |
| | | 2 | NP | — | 0 |
| | CCN-86R | 1 | NP | — | 0 |
| | | 2 | NP | — | 0 |

Injected material was visible in most injected (left) eyes. Injected silk fibroin hydrogel material was visible in two of three injected eyes; this material formed a mass up to 5 mm in diameter in the vitreous chamber, composed of pale amphophilic granular material surrounding 50-200 μm diameter pale basophilic structures with a more solid appearance. This material consistently lacked cellular infiltrates when captured. There were no other histologic findings in the silk fibroin hydrogel-injected eyes.

Injected material consistent with silk fibroin/celecoxib (CXB) rod was visible in two of the three injected eyes. This structure was present in the vitreous chamber, in close proximity to the retina; it was approximately 500 μm diameter, stained basophilic to amphophilic, and contained non-staining vacuoles or clefts. In one sample (CCN-86L, Block 1), the rod structure was surrounded and infiltrated by lymphocytes, macrophages, and multinucleated giant cells; however, in all other instances the rod was acellular. In two samples, the retina adjacent to the rod was focally distorted, with disorganized retinal layers and cell vacuolization. Given the proximity to the injected rod, this lesion was considered to be secondary to the injection procedure.

### Example 28. Physical Properties of Silk Fibroin Hydrogels with Celecoxib for In Vivo Studies

Silk fibroin hydrogels were prepared as described above. Briefly, lyophilized silk fibroin was reconstituted with an aqueous solution of sodium chloride, polysorbate-80, and phosphate buffer. The sodium chloride concentration was adjusted to ensure a final osmolarity of 280 mOsm. A suspension of celecoxib (CXB) was used to reconstitute silk fibroin in these hydrogel formulations. The silk fibroin was allowed to fully reconstitute prior to being drawn into a 6 mL syringe. Excipient solutions were prepared so that a 0.75:1 mix of silk-fibroin solution:excipient solution would result in the desired final formulations. The pH of polyethylene glycol (PEG) hydrogels was adjusted using hydrochloric acid to account for the changes in pH observed when mixing phosphate buffer and PEG. The excipient solutions were drawn up into a second 6 mL syringe. The solutions were mixed back and forth via a syringe connector until homogeneous. The resulting mixture was incubated at 37° C. overnight and aliquoted into 1 mL syringes prior to injection.

The formulations were prepared as described in Table 59. Multiple preparations of the same formulation may be examined. The samples in Table 59 were named by the process used to prepare and formulate each hydrogel. For example, the sample named “120 mb; hyd; 27.8% cxbst; 3% SFf; 10% CXBf; 10% P188f” refers to a formulation prepared from silk fibroin degummed with a 120-minute boil (120 mb), in a hydrogel format (hyd), from a stock of 27.8% w/v celecoxib (27.8% cxbst), with 3% w/v silk fibroin (3% SFf), with 10% w/v celecoxib (10% CXBf), and with 10% P188 (10% P188f). Longer boiling times (mb) produced silk fibroin with smaller molecular weights.

TABLE 59
Properties of hydrogel formulations with celecoxib

| Sample No. | 169-2 | 169-3 |
|---|---|---|
| Sample name | 120 mb; hyd; 27.8% | 480 mb; hyd; 27.8% |
| | cxbst; 3% SFf; 10% | cxbst; 3% SFf; 10% |
| | CXBf; 10% P188f | CXBf; 10% P188f |
| Description | 10% CXB; 3% 120 mb | 10% CXB; 3% 480 mb |
| | Silk; 10% Poloxamer-188 | Silk; 10% Poloxamer-188 |
| Average | 9.61 | 9.77 |
| Actual CXB % | | |
| Average CXB | 4.8 | 4.9 |
| dose (mg) | | |
| pH | 7.06 | 7.15 |
| Viscosity | 76.44 | 113.16 |
| (Pa*s) | | |
| Phase Angle | 5.35 | 8.68 |
| (°) | | |
| G′ (Pa) | 4487.2 | 9117.6 |
| G″ (Pa) | 418.9 | 1384.7 |
| Injection | 8.1 | 9.9 |
| force (N) | | |
| at 0.2 mL/ | | |
| minute | | |

### Rheological Analysis of Hydrogel Formulations

The rheological properties of the hydrogel samples were analyzed. Using a Bholin CVOR 150 rheometer, 800 μL of each sample was directly deposited onto a Peltier Plate system using a 25 mm diameter parallel plate. The oscillation method kept strain, temperature, and frequency constant at 0.1%, 25° C., and 1 Hz respectively. A time sweep was used to measure the G′, G″, and phase angle values over 150 seconds. The viscoelastic method kept the shear rate, strain, and frequency constant at 1 1/s, 0.1%, and 1 Hz respectively. A time sweep then measured the viscosity over 60 seconds. This was performed in triplicate for each sample.

The results of the experiments were shown in Table 59. The hydrogel with lower molecular weight silk fibroin (480 mb) had a higher viscosity and phase angle than the hydrogel with higher molecular weight silk fibroin (120 mb). Indeed, the viscosity of sample 169-3 (480 mb; hyd; 27.8% cxbst; 3% SFf; 10% CXBf; 10% P188f) was measured at 113.16 Pa*s, approximately 1.5 times greater than the measured viscosity of sample 169-2 (120 mb; hyd; 27.8% cxbst; 3% SFf; 10% CXBf; 10% P188f) at 76.44 Pa*s. The phase angle of sample 169-3 (480 mb; hyd; 27.8% cxbst; 3% SFf; 10% CXBf; 10% P188f) was 8.68°, approximately 1.6 times the phase angle of sample 169-2 (120 mb; hyd; 27.8% cxbst; 3% SFf; 10% CXBf; 10% P188f) at 5.35°.

The hydrogel with lower molecular weight silk fibroin (480 mb) also had a higher shear storage modulus and shear loss modulus than the hydrogel with higher molecular weight silk fibroin (120 mb). As used herein, the term “shear storage modulus” or “G′” refers to the measure of a material's elasticity or reversible deformation as determined by the material's stored energy. As used herein, the term “shear loss modulus” or “G″” refers to the measure of a material's ability to dissipate energy, usually in the form of heat. Sample 169-3 (480 mb; hyd; 27.8% cxbst; 3% SFf; 10% CXBf; 10% P188f) had a G′ value and a G″ value of 9117.6 Pa and 1384.7 Pa respectively. Sample 169-2 (120 mb; hyd; 27.8% cxbst; 3% SFf; 10% CXBf; 10% P188f) had a G′ value and a G″ value of 4487.2 Pa and 418.9 Pa respectively. The measured G′ for the lower molecular weight hydrogel was twofold greater than that of the higher molecular weight silk fibroin hydrogel, while the measured G″ was at least threefold greater than that of the higher molecular weight silk fibroin hydrogel. Ultimately, the use of lower molecular weight silk fibroin produced thicker, more viscous gels.

### Injection Forces

The force required to extrude the hydrogels was measured. Each hydrogel sample was mixed back and forth between two syringes to ensure homogeneity before being loaded into 1 mL syringe and capped with 27G, ½″ needles. The syringes were inserted into a Mark-10 syringe compression fixture and the test stand was set to move the head down onto the syringe plunger and extrude the hydrogel at a rate of 0.5 in/min. This was estimated to be equivalent to 0.2 mL/min with this syringe configuration. The force gauge measured the force required to extrude the hydrogel with a maximum force set at 200 N. Data was collected over 60 seconds (20 points per second) and exported and graphed to find where the injectability force plateau. The average value was taken over this plateau region. Each sample was injected in triplicate and average force measurements were calculated. The average force measurements were listed in Table 59. The average force for extrusion was measured to be 9.9 N for sample 169-3 (480 mb; hyd; 27.8% cxbst; 3% SFf; 10% CXBf; 10% P188f) and 8.1 N for sample 169-2 (120 mb; hyd 27.8% cxbst, 3% SFf; 10% CXBf; 10% P188f). Preparation from the lower molecular weight silk fibroin resulted in a stiffer hydrogel that required a greater force for extrusion.

### Example 29. Release of Protein Cargo from Silk Fibroin Hydrogels

Silk fibroin hydrogels were prepared from silk fibroin degummed with a 480 mb or a 120 mb. Sodium chloride was purchased from Chemsavers (Bluefield Va.). Polysorbate-80 was purchased from Croda (Snaith, United Kingdom). Phosphate buffered saline (10×PBS) was purchased from Gibco (USA). Sodium phosphate dibasic, sodium phosphate monobasic, human lysozyme, sucrose, Bovine Serum Albumin (BSA), trehalose, and poloxamer-188 (P188) were purchased from Sigma-Aldrich (St. Louis, Mo.). Sodium azide and glycerol were purchased from Fisher Chemical (Waltham, Mass.). Bevacizumab was purchased from Genentech Inc. (San Francisco, Calif.). Human immunoglobulin G (IgG) was purchased from Innovative Research (Novi, Mich.).

Silk Fibroin Hydrogel Preparation with Protein

To prepare the hydrogels with lysozyme, purified silk fibroin with a 480-minute boil or silk fibroin with a 120-minute boil were reconstituted to a concentration of 30% (w/v) with either water or lysozyme stock solution. The gelation excipient was mixed with these solutions to final formulation concentrations. The formulation was drawn into a syringe, capped, and left to gel at 4° C. overnight. Solutions that did not gel overnight were transferred to 37° C. for 3 hours to achieve gelling.

To prepare hydrogels with bovine serum albumin (BSA), 300 mg of purified silk fibroin with a 480-minute boil (mb) and silk fibroin degummed with a 120 mb was reconstituted with 0.7 mL of deionized water to make a final 30% (w/v) solution. BSA was dissolved with either polysorbate-80 (PS80) or poloxamer-188 (P188). Solutions were mixed to reach the desired final concentrations of fibroin/BSA/excipient. The resulting mixture was drawn into a 1 mL syringe, capped, and left to gel at 4° C. overnight. Solutions that did not gel overnight were transferred to 37° C. for 3 hours to achieve gelation.

To prepare hydrogels with bevacizumab, purified silk fibroin degummed with a 480-minute boil was reconstituted with sufficient deionized water to a concentration of 10% or 30% (w/v). Bevacizumab was lyophilized separately and re-dissolved in the silk solution. An 80% glycerol solution was mixed with the protein solution to obtain the final formulations. The resulting mixture was then drawn into a syringe, capped, and left to gel at 4° C. overnight. Solutions that did not gel were transferred to 37° C. for 3 hours to achieve gelling.

Purified 480 mb silk fibroin and 120 mb silk fibroin were reconstituted to 30% (w/v) with deionized water. IgG was dissolved with aqueous solutions of either polysorbate-80 (PS80) or P188. Solutions were mixed to reach the desired final concentrations of fibroin/IgG/excipient. The resulting mixture was drawn into a syringe, capped, and left to gel at 4° C. overnight. Solutions that did not gel overnight were transferred to 37° C. for 3 hours to achieve gelling.

The hydrogels prepared are described in Table 60. The samples were named for the process in which they were prepared. For example, the sample named “120 mb; hyd; 15% SFf; 2.5% bsaf; 10% P188f” refers to a sample prepared from silk fibroin degummed with a 120-minute boil (120 mb), a formulation as a hydrogel (hyd), a formulation with 15% w/v silk fibroin (15% SFf), a formulation with 2.5% w/v BSA (2.5% bsaf), and a formulation with 10% w/v P188 (10% P188f). Other potential components described included a formulation with lysozyme (% lysozymef), a preparation from silk fibroin degummed with a 480 mb (480 mb), a formulation with glycerol (% Glycf), a formulation with bevacizumab (% bevacizumabf), and a formulation with IgG (% iggf). Sample 203-03 (120 mb; hyd; 5% SFf; 2.5% lysozyme; 40% Glycf) did not form a gel. In Table 60, “Excip.” refers to excipient. All IgG hydrogels contained 0.01% polysorbate-80. All lysozyme hydrogels contained 0.01% polysorbate-80. Bevacizumab hydrogels contained trace amounts of that buffer in which it is provided (trehalose, a sodium phosphate buffer, and polysorbate-20). All BSA hydrogels contained 0.1% polysorbate-80.

TABLE 60
Preparations of silk fibroin hydrogels and controls with protein.

| | | | Silk | [Silk | | | | |
|---|---|---|---|---|---|---|---|---|
| Sample | | [Protein] | Fibroin | fibroin] | | [Excip.] | | Mass |
| No. | Protein | (%) | mb | (%) | Excip. | (%) | Sample name | (mg) |
| 203-01 | Lysozyme | 2.5 | 120 | 5 | P188 | 10 | 120 mb; hyd; | 145.37 |
| | | | | | | | 5% SFf; 2.5% | 182.93 |
| | | | | | | | lysozymef; | 202.12 |
| | | | | | | | 10% P188f | |
| 203-02 | Lysozyme | 10 | 120 | 5 | P188 | 10 | 120 mb; hyd; | 141.1 |
| | | | | | | | 5% SFf; 10% | 194.03 |
| | | | | | | | lysozymef | 163.08 |
| | | | | | | | 10% P188f | |
| 203-03 | Lysozyme | 2.5 | 120 | 5 | Glycerol | 40 | 120 mb; hyd; | — |
| | | | | | | | 5% SFf; 2.5% | — |
| | | | | | | | lysozymef; | — |
| | | | | | | | 40% Glvcf | |
| 203-04 | Lysozyme | 10 | 120 | 5 | Glycerol | 40 | 120 mb; hyd; | 213.35 |
| | | | | | | | 5% SFf; 10% | 218.72 |
| | | | | | | | lysozymef; | 217.83 |
| | | | | | | | 40% Glycf | |
| 203-05 | Lysozyme | 2.5 | 120 | 15 | P188 | 10 | 120 mb; hyd; | 173.35 |
| | | | | | | | 15% SFf; 2.5% | 207.02 |
| | | | | | | | lysozymef; | 199.35 |
| | | | | | | | 10% P188f | |
| 203-06 | Lysozyme | 10 | 120 | 15 | P188 | 10 | 120 mb; hyd; | 144.88 |
| | | | | | | | 15% SFf; 10% | 207.63 |
| | | | | | | | lysozymef; | 206.52 |
| | | | | | | | 10% P188f | |
| 203-07 | Lysozyme | 2.5 | 120 | 15 | Glycerol | 40 | 120 mb; hyd; | 223.87 |
| | | | | | | | 15% SFf; 2.5% | 205.3 |
| | | | | | | | lysozymef; | 218.84 |
| | | | | | | | 40% Glycf | |
| 203-08 | Lysozyme | 10 | 120 | 15 | Glycerol | 40 | 120 mb; hyd; | 152.39 |
| | | | | | | | 15% SFf; 10% | 207.88 |
| | | | | | | | lysozymef; | 207.87 |
| | | | | | | | 40% Glycf | |
| 203-09 | Lysozyme | 2.5 | 480 | 5 | P188 | 10 | 480 mb; hyd; | 248.13 |
| | | | | | | | 5% SFf; 2.5% | 191.39 |
| | | | | | | | lysozymef; | 207.16 |
| | | | | | | | 10% P188f | |
| 203-10 | Lysozyme | 2.5 | 480 | 5 | Glycerol | 40 | 480 mb; hyd; | 209.86 |
| | | | | | | | 5% SFf; 2.5% | 231.13 |
| | | | | | | | lysozymef; | 231.01 |
| | | | | | | | 40% Glycf | |
| 203-11 | Lysozyme | 2.5 | 480 | 15 | P188 | 10 | 480 mb; hyd, | 222.07 |
| | | | | | | | 15% SFf; 2.5% | 210.8 |
| | | | | | | | lysozymef; | 234.87 |
| | | | | | | | 10% P188f | |
| 203-12 | Lysozyme | 2.5 | 480 | 15 | Glycerol | 40 | 480 mb; hyd; | 280.46 |
| | | | | | | | 15% SFf; 2.5% | 223.15 |
| | | | | | | | lysozymef; | 232.37 |
| | | | | | | | 40% Glycf | |
| 197-01 | BSA | 2.5 | 480 | 5 | P188 | 10 | 480 mb; hyd; | 194.95 |
| | | | | | | | 5% SFf; 2.5% | 194.89 |
| | | | | | | | bsaf; 10% P188f | — |
| 197-02 | BSA | 2.5 | 480 | 15 | P188 | 10 | 480 mb; hyd; | 231.42 |
| | | | | | | | 15% SFf; 2.5% | 226.19 |
| | | | | | | | bsaf; 10% P188f | 277.73 |
| 197-03 | BSA | 2.5 | 120 | 5 | P188 | 10 | 120 mb; hyd; | 203.5 |
| | | | | | | | 5% SFf; 2.5% | 234.64 |
| | | | | | | | bsaf; 10% P188f | 227.49 |
| 197-04 | BSA | 2.5 | 120 | 15 | P188 | 10 | 120 mb; hyd; | 252.32 |
| | | | | | | | 15% SFf; 2.5% | 200.16 |
| | | | | | | | bsaf; 10% P188f | 217.13 |
| 197-05 | BSA | 2.5 | 120 | 5 | Glycerol | 40 | 120 mb; hyd; | 202.99 |
| | | | | | | | 5% SFf; 2.5% | 225.44 |
| | | | | | | | bsaf; 40% Glycf | 195.8 |
| 197-06 | BSA | 2.5 | 120 | 15 | Glycerol | 40 | 120 mb; hyd; | 338.31 |
| | | | | | | | 15% SFf; 2.5% | 206.25 |
| | | | | | | | bsaf; 40% Glycf | 214.3 |
| 187-2A | BSA | 2.5 | 480 | 5 | Glycerol | 40 | 480 mb; hyd; | 200.06 |
| | | | | | | | 5% SFf; 2.5% | 206.71 |
| | | | | | | | bsaf; 40% Glycf | 196.31 |
| 187-4A | BSA | 2.5 | 480 | 15 | Glycerol | 40 | 480 mb; hyd, | 204.86 |
| | | | | | | | 15% SFf; 2.5% | 207.03 |
| | | | | | | | bsaf, 40% Glycf | 196.56 |
| 201-01 | Bevacizumab | 2.5 | 480 | 5 | Glycerol | 40 | 480 mb; hyd; | 204.8 |
| | | | | | | | 5% SFf; 2.5% | 216.91 |
| | | | | | | | bevacizum | 224.1 |
| | | | | | | | abf; 40% Glycf | |
| 201-02 | Bevacizumab | 2.5 | 480 | 15 | Glycerol | 40 | 480 mb; hyd; | 222.22 |
| | | | | | | | 15% SFf; 2.5% | 225.4 |
| | | | | | | | bevacizum | 228.86 |
| | | | | | | | abf; 40% Glycf | |
| 201-03 | Bevacizumab | 2.5 | 120 | 5 | Glycerol | 40 | 120 mb; hyd; | 209.93 |
| | | | | | | | 5% SFf; 2.5% | 190.21 |
| | | | | | | | bevacizum | 226.91 |
| | | | | | | | abf; 40% Glycf | |
| 193-01 | IgG | 2.5 | 480 | 5 | P188 | 10 | 480 mb; hyd; | 204.54 |
| | | | | | | | 5% SFf; 2.5% | 197.27 |
| | | | | | | | iggf; 10% P188f | 196.44 |
| 193-02 | IgG | 2.5 | 120 | 5 | P188 | 10 | 120 mb; hyd; | 192.16 |
| | | | | | | | 5% SFf; 2.5% | 191.09 |
| | | | | | | | iggf; 10% P188f | 223.79 |
| 193-03 | IgG | 2.5 | 480 | 5 | Glycerol | 40 | 480 mb; hyd; | 201.41 |
| | | | | | | | 5% SFf; 2.5% | 220.71 |
| | | | | | | | iggf; 40% Glycf | 205.86 |
| 193-04 | IgG | 2.5 | 120 | 5 | Glycerol | 40 | 120 mb; hyd; | 194.62 |
| | | | | | | | 5% SFf; 2.5% | 195.54 |
| | | | | | | | iggf; 40% Glycf | 221.22 |
| 193-05 | IgG | 2.5 | 480 | 15 | P188 | 10 | 480 mb; hyd; | 192.2 |
| | | | | | | | 15% SFf; 2.5% | 208.87 |
| | | | | | | | iggf; 10% P188f | 226.44 |
| 193-06 | IgG | 2.5 | 120 | 15 | P188 | 10 | 120 mb; hyd; | 211.77 |
| | | | | | | | 15% SFf; 2.5% | 211.43 |
| | | | | | | | iggf; 10% P188f | 242.67 |
| 193-07 | IgG | 2.5 | 480 | 15 | Glycerol | 40 | 480 mb; hyd; | 228.47 |
| | | | | | | | 15% SFf; 2.5% | 211.99 |
| | | | | | | | iggf; 40% Glycf | 241.57 |
| 193-08 | IgG | 2.5 | 120 | 15 | Glycerol | 40 | 120 mb; hyd; | 190.38 |
| | | | | | | | 15% SFf; 2.5% | 215.59 |
| | | | | | | | iggf; 40% Glycf | 200.78 |
| 1,2-C | Control | N/A | 120 | 5 | P188 | 10 | 120 mb; hyd; | 242.33 |
| | sample | | | | | | 5% SFf; 10% | 183.7 |
| | | | | | | | P188f | — |
| 3,4-C | Control | N/A | 120 | 5 | Glycerol | 40 | 120 mb; hyd; | 205.27 |
| | sample | | | | | | 5% SFf; | 204.63 |
| | | | | | | | 40% Glycf | — |
| 5,6-C | Control | N/A | 120 | 15 | P188 | 10 | 120 mb; hyd; | — |
| | sample | | | | | | 15% SFf; | — |
| | | | | | | | 10% P188f | — |
| 7,8-C | Control | N/A | 120 | 15 | Glycerol | 40 | 120 mb; hyd; | 177.8 |
| | sample | | | | | | 15% SFf; | 229.1 |
| | | | | | | | 40% Glycf | — |
| 199-9 | BSA | 0.025 | N/A | N/A | Glycerol | 4 | 0.025% bsaf; | — |
| | | | | | | | 4% Glycf | — |
| | | | | | | | | — |
| 199-10 | BSA | 0.025 | N/A | N/A | P188 | 1 | 0.025% bsaf; | — |
| | | | | | | | 1% P188f | — |
| | | | | | | | | — |

In Vitro Release Profile of Silk Fibroin Hydrogels Formulated with Protein APIs and Other Excipients

Protein loaded silk-fibroin hydrogels were weighed in triplicate (at approximately 200 mg) into 4 mL vials. 2 mL of release media were added (PBS, 0.01% polysorbate-80, 0.05% sodium azide). Samples were incubated with gentle shaking at 37° C. At 2 hours, 4 hours, 1, 2, 3, 7, 9, 10, 14, 21, and 28 days, 150 μL of release media was removed and replaced with 150 μL of fresh media. Control samples containing 2.5% lysozyme, 2.5% IgG, 2.5% bevacizumab, or 2.5% BSA with either 4% glycerol or 1% P188 were prepared to serve as a 100% drug release control. Controls with protein and gelling agent were utilized to assess the effects of the gelling agent on protein stability. Total protein released was quantified via size-exclusion chromatography using a Waters X-Bridge Protein BEH SEC, 200 Å, 3.5 μm column. An isocratic flow of mobile phase (100 mM sodium phosphate, 200 mM NaCl, pH 6.8) was run at 0.80 mL/min to elute protein. The HPLC system used was an Agilent 1290 with a PDA detector. Protein elution was monitored at 280 and 214 nm using a PDA detector. Cumulative % released was calculated using theoretical loading. Control sample 5,6-C was not tested because it was too stiff to get out of the syringe. The results of the cumulative release studies could be seen in Table 61A and Table 61B. The samples or readings denoted with “*” were completed in duplicate and samples or reading denoted with “**” were completed in singlicate.

TABLE 61A
In vitro release of proteins from silk fibroin hydrogels; average cumulative
release percentage (%) of API released each day of measurment

| | Sample | Day | | | | | | | | |
|---|---|---|---|---|---|---|---|---|---|---|
| Protein | No. | 0 | 0.08 | 0.166 | 1 | 2 | 3 | 5 | 7 | 9 |
| Lysozyme | 203-1 | 0.0 | — | 84.575 | 71.01 | 95.24 | — | 96.25 | 121.36 | 108.19 |
| Lysozyme | 203-2 | 0.0 | — | 61.3 | 77.7 | 111.8 | — | 117.7 | 110.4 | 119.3 |
| Lysozyme | 203-4 | 0.0 | — | 60.0 | 65.6 | 97.6 | — | 95.5 | 88.6 | 95.8 |
| Lysozyme | 203-5 | 0.0 | — | 43.4 | 38.4 | 58.5 | — | 69.4 | 60.4 | 65.1 |
| Lysozyme | 203-6 | 0.0 | — | 76.4 | 79.8 | 110.8 | — | 110.9 | 107.9 | 120.8 |
| Lysozyme | 203-7 | 0.0 | — | 22.5 | 23.1 | 42.9 | — | 42.9 | 44.6 | 48.4 |
| Lysozyme | 203-8 | 0.0 | — | 32.4 | 73.1 | 80.4 | — | 85.7 | 82.9 | 89.7 |
| Lysozyme | 203-9 | 0.0 | 47.8 | — | 69.6 | 68.7 | 69.4 | — | 68.3 | — |
| Lysozyme | 203-10 | 0.0 | 83.1 | — | 74.0 | 81.8 | 82.7 | — | 83.0 | — |
| Lysozyme | 203-11 | 0.0 | 54.6 | — | 60.6 | 59.6 | 54.5** | — | 52.9 | — |
| Lysozyme | 203-12 | 0.0 | 25.3 | — | 24.4 | 20.7 | 20.4 | — | 19.5 | — |
| BSA | 197-1* | 0.0 | — | 66.0 | 131.9 | 109.1 | — | — | — | — |
| BSA | 197-2 | 0.0 | — | 90.4 | 123.2 | 105.3 | — | — | — | — |
| BSA | 197-3 | 0.0 | — | 73.0 | 122.9* | 89.7 | — | — | — | — |
| BSA | 197-4 | 0.0 | — | 96.2 | 136.9* | 108.4 | — | — | — | — |
| BSA | 197-5 | 0.0 | — | 77.6 | 121.6 | 116.9 | — | — | — | — |
| BSA | 197-6 | 0.0 | — | 94.3 | 103.2 | 86.6* | — | — | — | — |
| BSA | 187-2A | 0.0 | — | 52.6 | 74.6* | 75.7* | — | — | — | — |
| BSA | 187-4A | 0.0 | — | 70.8 | 69.1 | 68.0 | — | — | — | — |
| BSA | 199-9 | 0.0 | — | 107.9 | 118.9 | 119.7 | — | — | — | — |
| BSA | 199-10* | 0.0 | — | 107.4 | 124.8 | 125.6 | — | — | — | — |
| Bevacizumab | 201-1 | 0.0 | — | 36.8 | 46.6 | 48.6 | 46.7 | — | 43.4 | — |
| Bevacizumab | 201-2 | 0.0 | — | 39.1 | 45.7 | 43.7 | 36.7 | — | 31.5 | — |
| Bevacizumab | 201-3 | 0.0 | — | 46.4 | 72.5 | 63.1 | 57.0 | — | 66.6 | — |
| Bevacizumab | 201-5 | 0.0 | — | 89.9 | 108.8 | 89.2 | 83.4 | — | 86.6 | — |
| Bevacizumab | 201-6* | 0.0 | — | 90.0 | 103.8 | 92.4 | 88.9 | — | 92.7 | — |
| IgG | 193-01* | 0.0 | 24.1 | — | 35.6 | 30.4 | — | — | — | — |
| IgG | 193-02 | 0.0 | 9.8 | — | 13.8 | 12.7 | — | — | — | — |
| IgG | 193-03 | 0.0 | 56.1 | — | 70.3 | 59.1 | — | — | — | — |
| IgG | 193-04 | 0.0 | 50.4* | — | 62.5 | 50.7 | — | — | — | — |
| IgG | 193-05 | 0.0 | 40.0 | — | 46.7 | 45.3 | — | — | — | — |
| IgG | 193-06 | 0.0 | 29.0 | — | 28.5 | 27.3 | — | — | — | — |
| IgG | 193-07 | 0.0 | 43.0 | — | 42.0 | 38.1 | — | — | — | — |
| IgG | 193-08 | 0.0 | 61.1 | — | 44.9 | 43.2 | — | — | — | — |

TABLE 61B
Standard deviations of In vitro release of proteins from silk
fibroin hydrogels; standard deviations of average cumulative
release percentage (%) of API released each day of measurement

| | Sample | Day | | | | | | | | |
|---|---|---|---|---|---|---|---|---|---|---|
| Protein | No. | 0 | 0.08 | 0.166 | 1 | 2 | 3 | 5 | 7 | 9 |
| Lysozyme | 203-1 | 0.0 | — | 16.6 | 15.4 | 20.9 | — | 22.3 | 9.0 | 22.6 |
| Lysozyme | 203-2 | 0.0 | — | 3.9 | 9.4 | 16.2 | — | 10.2 | 16.1 | 17.6 |
| Lysozyme | 203-4 | 0.0 | — | 4.3 | 8.2 | 6.8 | — | 8.8 | 7.1 | 7.7 |
| Lysozyme | 203-5 | 0.0 | — | 16.4 | 15.1 | 21.3 | — | 4.2 | 19.2 | 21.0 |
| Lysozyme | 203-6 | 0.0 | — | 16.7 | 15.7 | 23.2 | — | 29.3 | 24.4 | 30.5 |
| Lysozyme | 203-7 | 0.0 | — | 2.4 | 5.0 | 1.8 | — | 1.9 | 2.2 | 2.3 |
| Lysozyme | 203-8 | 0.0 | — | 29.1 | 21.9 | 5.7 | — | 4.8 | 5.9 | 6.0 |
| Lysozyme | 203-9 | 0.0 | 5.0 | — | 13.2 | 13.6 | 11.2 | — | 12.7 | — |
| Lysozyme | 203-10 | 0.0 | 0.3 | — | 9.7 | 5.6 | 0.4 | — | 1.2 | — |
| Lysozyme | 203-11 | 0.0 | 3.7 | — | 2.7 | 3.8 | 0.0** | — | 1.4 | — |
| Lysozyme | 203-12 | 0.0 | 1.2 | — | 1.2 | 2.2 | 0.6 | — | 0.8 | — |
| BSA | 197-1* | 0.0 | — | 1.7 | 0.4 | 0.4 | — | — | — | — |
| BSA | 197-2 | 0.0 | — | 10.0 | 25.2 | 7.6 | — | — | — | — |
| BSA | 197-3 | 0.0 | — | 16.3 | 13.2* | 28.6 | — | — | — | — |
| BSA | 197-4 | 0.0 | — | 10.3 | 18.3* | 14.2 | — | — | — | — |
| BSA | 197-5 | 0.0 | — | 8.4 | 2.0 | 28.4 | — | — | — | — |
| BSA | 197-6 | 0.0 | — | 15.3 | 14.1 | 28.4* | — | — | — | — |
| BSA | 187-2A | 0.0 | — | 1.9 | 1.9* | 0.5* | — | — | — | — |
| BSA | 187-4A | 0.0 | — | 6.7 | 7.4 | 7.5 | — | — | — | — |
| BSA | 199-9 | 0.0 | — | 3.5 | 2.1 | 1.7 | — | — | — | — |
| BSA | 199-10* | 0.0 | — | 3.7 | 3.4 | 3.6 | — | — | — | — |
| Bevacizumab | 201-1 | 0.0 | — | 0.9 | 0.3 | 0.6 | 0.8 | — | 0.9 | — |
| Bevacizumab | 201-2 | 0.0 | — | 0.9 | 1.0 | 1.3 | 2.7 | — | 1.3 | — |
| Bevacizumab | 201-3 | 0.0 | — | 1.6 | 1.6 | 2.9 | 11.9 | — | 0.9 | — |
| Bevacizumab | 201-5 | 0.0 | — | 2.4 | 0.5 | 2.5 | 3.9 | — | 3.2 | — |
| Bevacizumab | 201-6* | 0.0 | — | 0.0 | 3.2 | 2.4 | 1.4 | — | 1.4 | — |
| IgG | 193-01* | 0.0 | 2.6 | — | 1.2 | 1.1 | — | — | — | — |
| IgG | 193-02 | 0.0 | 0.7 | — | 1.2 | 1.6 | — | — | — | — |
| IgG | 193-03 | 0.0 | 4.0 | — | 2.5 | 1.1 | — | — | — | — |
| IgG | 193-04 | 0.0 | 0.2 | — | 0.7 | 3.4 | — | — | — | — |
| IgG | 193-05 | 0.0 | 6.5 | — | 10.6 | 11.2 | — | — | — | — |
| IgG | 193-06 | 0.0 | 2 2 | — | 1.8 | 2.1 | — | — | — | — |
| IgG | 193-07 | 0.0 | 0.1 | — | 0.3 | 0.7 | — | — | — | — |
| IgG | 193-08 | 0.0 | 1.8 | — | 2.3 | 2.0 | — | — | — | — |

Lysozyme loading was used to modulate release kinetics. Formulations with lysozyme and P188 were analyzed first. Formulations prepared with P188 and 10% lysozyme loading and either 5% or 15% 120 mb silk fibroin day (203-2 and 203-6 respectively) reached nearly 80% release by 1 day. 120 mb silk fibroin hydrogel formulations with P188 showed silk fibroin concentration dependent API release. For example, sample 203-1 with 2.5% lysozyme and 5% 120 mb silk fibroin released 84.6% of the API in 4 hours. Increasing the silk fibroin concentration to 15% in sample 203-5 decreased the release at 4 hours to 43.4%, and caused the release to plateau at approximately 70% over 9 days.

In the hydrogels formulated with P188, the 5% 480 mb silk fibroin hydrogels with 2.5% lysozyme (203-9) showed lower burst and release when compared to the corresponding 120 mb silk fibroin hydrogels (203-1). The formulations with P188 and 480 mb silk fibroin also displayed a silk fibroin concentration dependence in release rate with silk fibroin concentration. This suggested that the release of lysozyme was related to the ratio of silk fibroin to lysozyme. The ratios of silk fibroin to lysozyme ranged from 0.5 to 6. In general, an increased ratio of silk fibroin to lysozyme reduced burst and release of the protein. Also, lower molecular weight silk fibroin may form a tighter hydrogel network, further reducing diffusion of the small lysozyme protein.

The release of lysozyme from silk hydrogels prepared with glycerol displayed similar trends to the those of the hydrogels prepared from P188. High loaded glycerol formulations (with 10% lysozyme) with 120 mb silk fibroin showed a high initial burst release dependent on silk fibroin concentration; higher concentrations of silk fibroin resulted in lower bursts of protein release. The formulation containing lower silk fibroin concentration (lower silk fibroin to lysozyme ratio) reached approximately 100% release at 2 days (sample 203-4), while the formulation containing higher concentration of silk fibroin plateaued at 80% and continued to release out to 9 days (sample 203-8). Increasing the silk fibroin to lysozyme ratio by reducing the lysozyme concentration from 10% to 2.5% reduced the initial burst (measured at 4 hours) from 32.4% in sample 203-8 to 22.5% in sample 203-7. This same effect can be seen with the 480 mb silk fibroin hydrogel formulations. Increasing the 480 mb silk fibroin concentration from 5% to 15%, while keeping the lysozyme loading constant at 2.5%, decreased the initial burst (measured at 2 hours) from 83.1% in sample 203-10 to 25.3% in sample 203-12. Lastly, hydrogels with glycerol and with the same silk fibroin to lysozyme ratio and different mb of silk fibroin showed similar release kinetics for the first day, however the 120 mb silk fibroin hydrogel (203-7) released at a faster rate over 9 days compared to the 480 mb silk fibroin hydrogel (203-12). The ratios of silk fibroin to lysozyme ranged from 0.5 to 6 for these hydrogels.

BSA loaded SF hydrogels showed very high burst and complete release of the protein within 1-3 days. BSA loaded silk fibroin hydrogels made with P188 as a gelling excipient reached complete release within 1 day. 4 hours into the experiment, cumulative release percentages ranged from approximately 66% to approximately 96%. The ratios of silk fibroin to BSA ranged from 2 to 6. Silk fibroin molecular weight or concentration, in the ranges tested, did not affect release kinetics of BSA in the hydrogel formulations with P188. The BSA control sample showed no reduction in concentration over the course of the study. In vitro release data for hydrogels prepared with glycerol showed that hydrogels made with 120 mb silk fibroin had a higher burst release and reached 100% release more quickly than 480 mb silk fibroin hydrogels. 480 mb silk fibroin hydrogels release approximately 65-80% of BSA by day 1, but the release then plateaus at day 2. Control BSA solution showed stability over the 2 days of release testing. This relationship between silk fibroin molecular weight and release of protein could represent a size dependent release mechanism. Protein release was diffusion based. Since there is minimal hydrolysis and no added enzymes, little to no degradation of the silk fibroin matrix occurs in vitro. Therefore, decreased release kinetics might be due to a tighter hydrogel network impeding the release of BSA. This effect was not observed with the P188 formulations. The hydrogel network might be different with the different gelling agents.

IgG release kinetics from silk fibroin hydrogel formulations with glycerol varied between 38.1% to 59.1% over two days, without significant release following measured cumulative API release at 2 hours. Hydrogels made with 5% silk fibroin (samples 193-03 and 193-04) released more protein by 2 days than those made with 15% silk fibroin (samples 193-07 and 193-08) regardless of the boiling time and molecular weight of the silk fibroin. This result indicated that the silk fibroin to IgG ratio could play a role in diffusion of protein from the silk fibroin formulation. Hydrogels prepared with 5% silk fibroin had a silk fibroin to IgG ratio of 2, while hydrogels prepared with 15% silk fibroin had a silk fibroin to IgG ratio of 6. Hydrogel formulations prepared with P188 demonstrated lower bursts and released less IgG (maximum release was 45.3%) than those made with glycerol (maximum release 59.1%). In general, by two days hydrogels made with 480 mb silk fibroin released more IgG than those made with 120 mb silk fibroin. Interestingly, 15% silk fibroin hydrogels made with P188 released more IgG than the corresponding hydrogels made with 5% silk fibroin, which was the opposite trend observed for the glycerol gels. A hazy precipitate also formed during formulation of the hydrogels with P188.

Bevacizumab release kinetics from silk fibroin hydrogel formulations all had similar characteristics. Hydrogels prepared with 5% silk fibroin had a silk fibroin to bevacizumab ratio of 2, while hydrogels prepared with 15% silk fibroin had a silk fibroin to bevacizumab ratio of 6. There was an initial burst phase, followed by a plateau. The burst release varied dependent upon molecular weight of the silk fibroin. 480 mb silk fibroin formulations showed lower initial bursts (measured at 4 hours) of approximately 40% while 120 mb silk fibroin formulations showed initial bursts (measured at 4 hours) of 46.4%. The difference increased at 1 day of release. The formulations with 480 mb silk fibroin (201-1 and 201-2) had released approximately 45% of the protein, while the formulation with 120 mb silk fibroin (201-3) had released 72.5% of its bevacizumab. The lower burst and lack of release with the 480 mb silk fibroin formulations could be due to a tighter silk network that formed with shorter silk fibroin proteins compared to the larger 120 mb silk fibroin hydrogels. There was no difference in release kinetics between formulations with 480 mb silk fibroin concentrations between 5 and 15%. The bevacizumab control displayed that the protein was stable in release media at 37° C. with only a 10% loss maintained over 7 days.

In general, silk fibroin hydrogels showed higher burst and faster release kinetics than the corresponding rod formulations. When compared to BSA, bevacizumab, and IgG hydrogel formulations, lysozyme (14.7 kDa) released faster than the much larger bevacizumab and IgG molecules (approximately 160 kDa) but more slowly than BSA. Bevacizumab loaded hydrogels containing glycerol released similar levels of protein (45%-70%) as IgG loaded hydrogels with glycerol. Both IgG and bevacizumab loaded hydrogels showed decreased release rate with increasing silk fibroin concentration. Given the similar size of these proteins (both approximately 150 kDa), it was possible that the release was controlled by diffusion through the silk fibroin network. BSA (66.5 kDa) and lysozyme (14.7 kDa) hydrogels released 100% of the protein by day 2, which suggested that smaller proteins diffused more quickly through the silk fibroin hydrogel network.

### Example 30. Rheological Properties of Silk Fibroin Hydrogels with Celecoxib

The rheological properties of hydrogels loaded with celecoxib (CXB) were studied. The formulations were prepared as described for the cumulative release studies of celecoxib from silk fibroin hydrogels, seen in Table 62. To study the rheology, 600 μL of each hydrogel sample was loaded onto the Peltier plate of a Bholin CVOR 150 rheometer. Samples were analyzed at 25° C. using a 20 mm parallel plate and a gap of 1.0 mm. Oscillation parameters were set at a frequency of 1 Hz and 0.01% strain. Viscosity was measured at a shear rate of 1 1/s for 135 seconds, as seen in Table 62. Samples in Table 62 were named by the process used to prepare and formulate each hydrogel. For example, in the sample named 120 mb; hyd; 27.8% cxbst: 5% SFf; 10% CXBf: 40% PEG4kf, “120 mb” refers to silk degummed with a 120-minute boil, “hyd” refers to the formulation of the sample as a hydrogel, “27.8% cxbst” refers to a preparation from a stock solution of 27.8% of celecoxib. “5% SFf” refers to a formulation with 5% (w/v) silk fibroin. “10% CXBf” refers to a formulation with 10% (w/v) celecoxib, and “40% PEG4kf” refers to a formulation with 40% PEG 4 kDa. Some hydrogels were prepared with P188 (% P188f).

TABLE 62
Rheology data for hydrogel formulations with celecoxib.
Std. Dev. refers to standard deviation.

| | | | | | Phase | | | | |
|---|---|---|---|---|---|---|---|---|---|
| | | | | Phase | Angle | | G′ | | G″ |
| Sample | | Viscosity | Viscosity | Angle | Std. | G′ | Std. | G″ | Std. |
| No. | Sample name | (Pas) | Std.Dev. | (°) | Dev. | (Pa) | Dev. | (Pa) | Dev. |
| 168-1 | 120 mb; | 964.19 | 182.55 | 10.80 | 0.54 | 31982 | 1516 | 6086 | 74 |
| | hyd; 27.8% | | | | | | | | |
| | cxbst; 5% | | | | | | | | |
| | SFf; 10% | | | | | | | | |
| | CXBf; 40% | | | | | | | | |
| | PEG4kf | | | | | | | | |
| 168-2 | 120 mb; | 324.48 | 50.86 | 9.86 | 1.65 | 7668 | 678 | 1316 | 82 |
| | hyd; 27.8% | | | | | | | | |
| | cxbst; 3% | | | | | | | | |
| | SFf; 10% | | | | | | | | |
| | CXBf; 40% | | | | | | | | |
| | PEG4kf | | | | | | | | |
| 168-3 | 120 mb; | 484.94 | 13.86 | 8.32 | 2.72 | 30246 | 2656 | 4328 | 810 |
| | hyd; 27.8% | | | | | | | | |
| | cxbst; 5% | | | | | | | | |
| | SFf; 10% | | | | | | | | |
| | CXBf; 10% | | | | | | | | |
| | P188f | | | | | | | | |
| 168-4 | 120 mb; | 76.44 | 5.60 | 5.35 | 0.42 | 4487 | 274 | 419 | 22 |
| | hyd; 27.8% | | | | | | | | |
| | cxbst; 3% | | | | | | | | |
| | SFf; 10% | | | | | | | | |
| | CXBf; 10% | | | | | | | | |
| | P188f | | | | | | | | |
| 168-5 | 480 mb; | 238.18 | 68.89 | 9.98 | 2.20 | 3545 | 497 | 609 | 57 |
| | hyd; 27.8% | | | | | | | | |
| | cxbst; 5% | | | | | | | | |
| | SFf; 10% | | | | | | | | |
| | CXBf; 40% | | | | | | | | |
| | PEG4kf | | | | | | | | |
| 168-6 | 480 mb; | 43.55 | 14.96 | 11.79 | 1.54 | 503 | 67 | 103 | 3 |
| | hyd; 27.8% | | | | | | | | |
| | cxbst; 3% | | | | | | | | |
| | SFf; 10% | | | | | | | | |
| | CXBf; 40% | | | | | | | | |
| | PEG4kf | | | | | | | | |
| 168-7 | 480 mb; | 307.25 | 15.35 | 8.75 | 0.28 | 30825 | 1609 | 4737 | 153 |
| | hyd; 27.8% | | | | | | | | |
| | cxbst; 5% | | | | | | | | |
| | SFf; 10% | | | | | | | | |
| | CXBf; 10% | | | | | | | | |
| | P188f | | | | | | | | |
| 168-8 | 480 mb; | 113.16 | 9.29 | 8.68 | 0.93 | 9118 | 667 | 1385 | 94 |
| | hyd; 27.8% | | | | | | | | |
| | cxbst; 3% | | | | | | | | |
| | SFf; 10% | | | | | | | | |
| | CXBf; 10% | | | | | | | | |
| | P188f | | | | | | | | |
| 168-9 | 480 mb; | 59.72 | 5.47 | 8.14 | 0.53 | 3353 | 203 | 478 | 7 |
| | hyd; 27.8% | | | | | | | | |
| | cxbst; 2% | | | | | | | | |
| | SFf; 10% | | | | | | | | |
| | CXBf; 10% | | | | | | | | |
| | P188f | | | | | | | | |

The viscosity of the silk fibroin hydrogels was directly related to both the concentration of silk fibroin and the molecular weight of the silk fibroin in the hydrogel. Higher concentrations of silk fibroin and/or the use of silk fibroin with a higher average molecular weight yielded higher viscosities in otherwise identical formulations. In formulations with 120 mb silk fibroin, the viscosity was lower for formulations with P188 instead of PEG 4 kDa. For formulations with 480 mb silk fibroin, the viscosity was higher for formulations with P188 instead of PEG 4 kDa. Formulations with P188 also had a smaller phase angle than the corresponding formulation with PEG 4 kDa. The concentration of silk fibroin in a hydrogel demonstrated a direct relationship with the stiffness of the hydrogel, as evidenced by the measured by the storage modulus (G′) and the loss modulus (G″). Both the G′ and G″ values increased with increasing concentrations of silk fibroin.

### Example 31. Injectability of Silk Fibroin Hydrogels with Celecoxib

The formulations were prepared as described for the cumulative release studies of celecoxib from silk fibroin hydrogels, seen in Table 63. The force required to extrude the hydrogels (injection force) was measured. Each hydrogel sample was mixed back and forth between two syringes to ensure homogeneity before being loaded into 1 mL syringe and capped with 27G, ½″ needles. The syringes were inserted into a Mark-10 syringe compression fixture and the test stand was set to move the head down onto the syringe plunger and extrude the hydrogel at a rate of 0.5 in/min. This was estimated to be equivalent to 0.2 m/min with this syringe configuration. The force gauge measured the force required to extrude the hydrogel with a maximum force set at 200 N. Data was collected over 60 seconds (20 points per second) and exported and graphed to find where the injectability force plateaued. The average value was taken over this plateau region. Each sample was injected in duplicate and average injection force measurements were calculated.

TABLE 63
Analysis of the injectability of silk fibroin
hydrogel formulations with celecoxib

| | | Injection force (N) at 0.2 mL/minute | | | |
|---|---|---|---|---|---|
| Sample | | Average | Average | Overall | Standard |
| No. | Sample name | 1 | 2 | Average | Deviation |
| 168-1 | 120 mb; hyd; 27.8% | 43.5 | 43.2 | 43.4 | 0.2 |
| | cxbst; 5% SFf; 10% | | | | |
| | CXBf; 40% PEG4kf | | | | |
| 168-2 | 120 mb; hyd; 27.8% | 19.6 | 20.4 | 20.0 | 0.6 |
| | cxbst; 3% SFf; 10% | | | | |
| | CXBf; 40% PEG4kf | | | | |
| 168-3 | 120 mb; hyd; 27.8% | 16.5 | 15.2 | 15.9 | 0.9 |
| | cxbst; 5% SFf; 10% | | | | |
| | CXBf; 10% P188f | | | | |
| 168-4 | 120 mb; hyd; 27.8% | 7.3 | 8.9 | 8.1 | 1.2 |
| | cxbst; 3% SFf; 10% | | | | |
| | CXBf; 10% P188f | | | | |
| 168-5 | 480 mb; hyd; 27.8% | 21.3 | 21.9 | 21.6 | 0.4 |
| | cxbst; 5% SFf; 10% | | | | |
| | CXBf; 40% PEG4kf | | | | |
| 168-6 | 480 mb; hyd; 27.8% | 9.6 | 9.6 | 9.6 | 0.0 |
| | cxbst; 3% SFf; 10% | | | | |
| | CXBf; 40% PEG4kf | | | | |
| 168-7 | 480 mb; hyd; 27.8% | 16.1 | 16.9 | 16.5 | 0.5 |
| | cxbst; 5% SFf; 10% | | | | |
| | CXBf; 10% P188f | | | | |
| 168-8 | 480 mb; hyd; 27.8% | 9.4 | 10.3 | 9.9 | 0.7 |
| | cxbst; 3% SFf; 10% | | | | |
| | CXBf; 10% P188f | | | | |
| 168-9 | 480 mb; hyd; 27.8% | 6.0 | 6.4 | 6.2 | 0.3 |
| | cxbst; 2% SFf; 10% | | | | |
| | CXBf; 10% P188f | | | | |

The experimental results demonstrated a direct relationship between the concentration of silk fibroin in a hydrogel and the injection force the silk fibroin hydrogel required. Hydrogels with a higher concentration of silk fibroin (e.g. sample 168-1) required a larger injection force to extrude the hydrogel than the corresponding formulation with a lower concentration of silk fibroin (e.g. 168-2). In general, the hydrogels prepared with PEG 4 kDa required higher injection forces than the corresponding hydrogel with P188. In addition, the molecular weight of silk fibroin in the hydrogel was directly related to the injection force in the hydrogels prepared with PEG 4 kDa. The PEG 4 kDa hydrogels prepared from higher molecular weight silk fibroin (120 mb) demonstrated a higher injection force than the corresponding hydrogels prepared from comparatively lower molecular weight silk fibroin (480 mb).

### Example 32. Effect of Select Excipients on Physical Properties of Hydrogels

The injectability experiment as described above was repeated to evaluate the effect of different excipients on injectability. Silk fibroin was degummed as described above, with a 120 mb. Glycerol was purchased from Fisher Chemical (Waltham, Mass.). Celecoxib (CXB) was purchased from Cipla, Miami Fla. Polysorbate-80 was purchased from Croda (Snaith UK). Potassium phosphate monobasic and potassium phosphate dibasic were purchased from Sigma Aldrich Fine Chemical (SAFC, St. Louis Mo.).

### Preparation of Silk Fibroin Hydrogels

To prepare the hydrogels with glycerol, 300 mg of the 120 mb silk fibroin was dissolved in a 20% w/v stock suspension of dry heat treated (DHT) CXB with polysorbate-80 and phosphate buffer to prepare a silk/CXB suspension with either 7.1% (w/v) or 8.8% (w/v) silk fibroin. The suspensions with higher concentration of silk fibroin were used to generate the hydrogels with higher concentrations of silk fibroin. 2.835 mL of the resulting silk/CXB suspension was added to a 6 mL syringe. The silk/CXB suspension was then mixed with a second syringe containing 2.165 mL of a 92.4% w/v stock solution of glycerol via a B Braun fluid dispensing connector, back and forth until homogeneous (at least 25 times). The resulting mixture was then capped with a sterile syringe cap and incubated on a rotator overnight at 37° C. The syringes were stored at 4° C. until use.

To prepare the hydrogels with PEG400, 300 mg of the 120 mb silk fibroin was dissolved in a 20% w/v stock suspension of dry heat treated (DHT) CXB with polysorbate-80 and phosphate buffer to prepare a silk/CXB suspension with either 7.1% (w/v) or 8.8% (w/v) silk fibroin. The suspensions with higher concentration of silk fibroin were used to generate the hydrogels with higher concentrations of silk fibroin. 2.835 mL of the resulting silk/CXB suspension was added to a 6 mL syringe. The silk/CXB suspension was then mixed with a second syringe containing 2.165 mL of a 92.4% w/v stock solution of PEG400 via a B Braun fluid dispensing connector, back and forth until homogeneous (at least 25 times). The resulting mixture was then capped with a sterile syringe cap and incubated on a rotator overnight at 37° C. The syringes were stored at 4° C. until use.

The formulations were prepared as described in Table 64. The formulations tested were named by the method in which they were prepared. For example, in the sample named “120 mb; hyd; 20% cxbst; 4% SFf; 10% CXBf; 40% Glycf”, “120 mb” refers to silk degummed with a 120-minute boil, “hyd” refers to the formulation of the sample as a hydrogel, “20% cxbst” refers to a preparation from a stock solution of 20% of celecoxib, “4% SFf” refers to a formulation with 4% (w/v) silk fibroin, “10% CXBf” refers to a formulation with 10% (w/v) celecoxib, and “40% Glycf” refers to a formulation with 40% glycerol. PEG400 was denoted in the hydrogels with “PEG400f”.

TABLE 64
Silk fibroin hydrogels with PEG400 or glycerol as excipients

| Sample | % Silk | | % | |
|---|---|---|---|---|
| No. | Fibroin | Excipient | Excipient | Sample Name |
| 158-1 | 4 | Glycerol | 40 | 120 mb; hyd; 20% cxbst; |
| | | | | 4% SFf; 10% CXBf; 40% |
| | | | | Glycf |
| 158-2 | 4 | PEG400 | 40 | 120 mb; hyd; 20% cxbst; |
| | | | | 4% SFf; 10% CXBf; 40% |
| | | | | PEG400f |
| 158-3 | 5 | Glycerol | 40 | 120 mb; hyd; 20% cxbst; |
| | | | | 5% SFf; 10% CXBf; 40% |
| | | | | Glycf |
| 158-4 | 5 | PEG400 | 40 | 120 mb; hyd; 20% cxbst; |
| | | | | 5% SFf; 10% CXBf; 40% |
| | | | | PEG400f |

Injectability of Silk Fibroin Hydrogels with Select Excipients

The hydrogel samples were loaded into 1 mL syringes. The syringe was capped with a 27-gauge needle and loaded onto a Mark-10 syringe compression fixture. The test stand was set to extrude the hydrogel at a rate of 0.5 inches per minute, which was estimated to be equivalent to 0.2 mL/min. The force gauge then measured the force required to extrude the hydrogel at that rate, with a maximum force set at 200 N. The injection forces required to extrude the hydrogel at this rate were measured over 60 seconds, with 20 points per second. The data was then exported and graphed to find where the injectability plateaus. The average value was taken over this range. The results were presented in Table 65. The data showed that using PEG400 as an excipient led to approximately 25% greater resistance for injection than glycerol. The hydrogels with glycerol had lower injection forces than the corresponding hydrogel with PEG400 at all concentrations tested. It was also observed that hydrogels with 5% silk fibroin required higher injection forces than hydrogels with 4% silk fibroin, which was consistent with previous observations. All of the hydrogels created were within the acceptable injectability range.

TABLE 65
Injectability measurements with different excipients

| | | | | Replicate 1 | | Replicate 2 | | Overall | |
|---|---|---|---|---|---|---|---|---|---|
| Sample | % Silk | | % | Average | Standard | Average | Standard | Average | Standard |
| No. | Fibroin | Excipient | Excipient | Force (N) | Dev. | Force (N) | Dev. | Force (N) | Dev. |
| 158-1 | 4 | Glycerol | 40 | 7.95 | 0.17 | 8.12 | 0.26 | 8.03 | 0.12 |
| 158-2 | 4 | PEG400 | 40 | 9.85 | 0.12 | 10.53 | 0.15 | 10.19 | 0.48 |
| 158-3 | 5 | Glycerol | 40 | 14.57 | 0.23 | 14.59 | 0.28 | 14.58 | 0.01 |
| 158-4 | 5 | PEG400 | 40 | 18.97 | 0.34 | 18.51 | 0.13 | 18.74 | 0.33 |

Rheology of Silk Fibroin Hydrogels with Select Excipients

The hydrogel samples were loaded onto a Peltier plate system that kept the temperature at 25° C. The geometry used was a 20 mm parallel plate. The gap was set at 1 mm and the frequency at 1 Hz. Viscosity was taken during a time sweep at 11/s over 135 seconds. The experimental results were presented in Table 66. In hydrogels having the same silk fibroin concentration, using glycerol as an excipient created more viscous hydrogels than using PEG400. The effect was more prominent in hydrogels with 4% silk fibroin than 5%. The glycerol samples were generally stiffer than the PEG400 hydrogels at these two silk fibroin concentrations as measured by viscosity. However, the glycerol hydrogels also had lower injection forces at both concentrations. This difference indicated that either the glycerol has a positive effect on injectability, or PEG400 has a negative effect, or some combination thereof. The glycerol hydrogels could also exhibit more pronounced shear-thinning behavior than PEG400 hydrogels. This would account for the lower injection force when under greater shear stress. The more viscous samples were more likely to be the most cohesive hydrogels in vivo.

TABLE 66
Viscosity measurements with different excipients

| | | | | Replicate 1 | | Replicate 2 | | Overall | |
|---|---|---|---|---|---|---|---|---|---|
| | | | | Average | | Average | | Average | |
| Sample | % Silk | | % | Viscosity | Standard | Viscosity | Standard | Viscosity | Standard |
| No. | Fibroin | Excipient | Excipient | (Pa*s) | Dev. | (Pa*s) | Dev. | (Pa*s) | Dev. |
| 158-1 | 4 | Glycerol | 40 | 103.97 | 2.43 | 138.58 | 6.82 | 121.27 | 24.47 |
| 158-2 | 4 | PEG400 | 40 | 62.71 | 4.56 | 60.47 | 4.54 | 61.59 | 1.58 |
| 158-3 | 5 | Glycerol | 40 | 231.07 | 15.85 | 281.12 | 16.07 | 256.10 | 35.39 |
| 158-4 | 5 | PEG400 | 40 | 207.57 | 11.17 | 219.53 | 13.12 | 213.55 | 8.46 |

### Example 33. Analysis of Solutions Prepared from Lyophilized Silk Fibroin with Varying Buffers and Freezing Conditions

The effects of different buffers and freezing conditions on the lyophilization of silk fibroin were determined. Silk yarn (Jiangsu SOHO Silk and Textile Co.) was degummed at 100° C. for 480 minutes in 0.02 M sodium carbonate solution (sodium carbonate was purchased from Fisher Bioreagents), followed by three warm (65° C.) and room temperature (RT) washes in MilliQ® water. The resulting fibroin was dried overnight at RT, weighed, and dissolved at 20% (w/v) in 9.3 M lithium bromide solution for five hours at 60° C. (lithium bromide was purchased from Fisher Chemical, Waltham Mass.). This solution was dialyzed against MilliQ® water in 50 kDa regenerated cellulose membrane (Spectra/Por, CAS: 131384, Lot: 3282822) for 48 hours at 4° C. with 6 water exchanges. The solution was centrifuged for 20 minutes at 3,900 RPM (on a benchtop Eppendorf refrigerated centrifuge) and 4° C. to remove insoluble particles. The concentration of the resulting solution was then determined using a UV absorbance assay (280 nm), and the appropriate amount of buffer was added to obtain a final concentration of 30 mg/ml silk fibroin (3% w/v). Multiple conditions were assessed, including 2 mM histidine (histidine was purchased from Sigma-Aldrich, St. Louis, Mo.), 10 mM histidine, 10 mM phosphate buffer (PB) (potassium phosphate monobasic and potassium phosphate dibasic were purchased from SAFC, St. Louis Mo.), and 1% sucrose (Sigma-Aldrich, St. Louis, Mo.) with 2 mM histidine. Final 30 mg/ml solutions were filtered through a 0.2 μm PES membrane prior to aliquoting and freezing. Under aseptic solutions, filtered solutions were aliquoted into 50 mL conical tubes (10 mL per tube), covered with Steri-Wrap®, and frozen in one of two ways. In the first way, tubes containing silk fibroin were placed at −80° C. for 16 hours (overnight). In the second way tubes containing silk fibroin were first placed in liquid nitrogen for 10 minutes, and then transferred to −80° C. overnight. All tubes were then lyophilized in a manifold freeze dryer (Labonco Freezone 4.5) for 72 hours. The preparations of lyophilized silk fibroin were presented in Table 67.

TABLE 67
Lyophilized silk fibroin preparations

| | | | Volume of | Volume | Volume | Volume | Volume | |
|---|---|---|---|---|---|---|---|---|
| | | | 3% silk | of silk | of 100 mM | of 100 mM | of 25% | Volume |
| | | Freezing | solution to | fibroin | PB buffer | Histidine | sucrose | of water |
| Sample | Buffer | condition | be made (mL) | (mL) | to add (mL) | to add (mL) | to add (mL) | to add (mL) |
| 77-A | 2 mM | Liquid | 105 | 36.67 | 0 | 2.1 | 0 | 66.23 |
| | Histidine | Nitrogen. | | | | | | |
| 77-B | 2 mM | −80° C. | 105 | 36.67 | 0 | 2.1 | 0 | 66.23 |
| | Histidine | | | | | | | |
| 77-C | 10 mM | Liquid | 105 | 36.67 | 0 | 10.5 | 0 | 57.83 |
| | Histidine | Nitrogen | | | | | | |
| 77-D | 10 mM | −80° C. | 105 | 36.67 | 0 | 10.5 | 0 | 57.83 |
| | Histidine | | | | | | | |
| 77-E | 10 mM PB | Liquid | 105 | 36.37 | 10.5 | 0 | 0 | 57.83 |
| | | Nitrogen | | | | | | |
| 77-F | 10 mM PB | −80° C. | 105 | 36.37 | 10.5 | 0 | 0 | 57.83 |
| 77-G | 1% Sucrose, | Liquid | 105 | 36.67 | 0 | 2.1 | 4.2 | 62.03 |
| | 2 mM | Nitrogen | | | | | | |
| | Histidine | | | | | | | |
| 77-H | 1% Sucrose, | −80° C. | 105 | 36.67 | 0 | 2.1 | 4.2 | 62.03 |
| | 2 mM | | | | | | | |
| | Histidine | | | | | | | |
| 77-I | 1% Sucrose, | Liquid | 120 | 65.17 | 0 | 2.4 | 4.8 | 47.61 |
| | 2 mM | Nitrogen | | | | | | |
| | Histidine, | | | | | | | |
| | 4.7% Silk | | | | | | | |
| | Fibroin | | | | | | | |
| 77-J | 1% Sucrose, | −80° C. | 120 | 65.17 | 0 | 2.4 | 4.8 | 47.61 |
| | 2 mM | | | | | | | |
| | Histidine, | | | | | | | |
| | 4.7% Silk | | | | | | | |
| | Fibroin | | | | | | | |

### Analysis of Reconstitution Efficiencies of Silk Fibroin Solutions

Silk fibroin from each condition was reconstituted at 300% (w/v) silk fibroin and left to dissolve for 30 minutes at 37° C. As used herein, the term “reconstitution efficiency” refers to the percentage of lyophilized, processed silk dissolved in a solution. The processed silk may be silk fibroin. The percentage may be calculated from the amount of silk fibroin successfully dissolved as compared to the total amount of silk fibroin intended to be dissolved ((Actual concentration in mg/mL)/(Theoretical concentration in mg/mL)×100%). Reconstitution efficiency was determined by measuring the absorbance of these solutions at 280 nm compared to a standard curve of known silk fibroin concentration on a SpectraMax i3x. The absorbance at 280 nm of each sample, the set-up of the samples within the plate, the sample dilutions of the plate (Corning 96-well flat-bottom UV well), the calculated concentrations of silk fibroin for each sample, and the dilution corrected calculation of the concentration of silk fibroin in solution were shown in Table 68. The calculated concentrations of silk fibroin were solved for from the line of best fit for the standard curve (y=4.619030415x+0.019085714; wherein x represented the concentration of silk fibroin and y represented the absorbance at 280 nm). The R² value of this line was determined to be 0.999168142. The dilution corrected concentration was determined by multiplying the calculated concentration by the dilution factor listed in the plate setup (e.g. 100×).

TABLE 68
Experimental data from the calculation of silk fibroin concentration in reconstituted silk fibroin solutions

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

A280 Absorbance

| | | | | | | | | | | | |
|---|---|---|---|---|---|---|---|---|---|---|---|
| 2.352 | 1.132 | 0.584 | 0.306 | 0.170 | 0.044 | 1.383 | 0.706 | 1.306 | 0.658 | 1.364 | 0.699 |
| 1.323 | 0.671 | 1.366 | 0.686 | 0.039 | 0.039 | 1.368 | 0.692 | 1.363 | 0.695 | 1.416 | 0.726 |
| 1.413 | 0.711 | 1.396 | 0.707 | 1.435 | 0.729 | 1.327 | 0.678 | 1.349 | 0.684 | 1.288 | 0.674 |
| 1.347 | 0.684 | 1.848 | 0.945 | 1.872 | 0.941 | 1.832 | 0.928 | 1.939 | 0.977 | 0.039 | 0.039 |

Plate Setup

| Std curve (mg/mL SF) | | | | | | | | | | | |
|---|---|---|---|---|---|---|---|---|---|---|---|
| 0.5 | 0.25 | 0.125 | 0.0625 | 0.0313 | 0 | A-A | A-A | A-B | A-B | B-A | B-A |
| | | | | | | 100x | 200x | 100x | 200x | 100x | 200X |
| B-B | B-B | C-A | C-A | — | — | D-A | D-A | D-B | D-B | E-A | E-A |
| 100X | 200X | 100X | 200X | | | 100X | 200X | 100X | 200X | 100X | 200X |
| E-B | E-B | F-A | F-A | F-B | F-B | G-A | G-A | G-B | G-B | H-A | H-A |
| 100X | 200X | 100X | 200X | 100X | 200X | 100X | 200X | 100X | 200X | 100X | 200X |
| H-B | H-B | I-A | I-A | I-B | I-B | J-A | J-A | J-B | J-B | — | — |
| 100X | 200X | 100X | 200X | 100X | 200X | 100X | 200X | 100X | 200X | | |

Calculated Concentration of Silk Fibroin (mg/mL)

| | | | | | | | | | | | |
|---|---|---|---|---|---|---|---|---|---|---|---|
| — | — | — | — | — | — | 0.2953 | 0.1486 | 0.2786 | 0.1382 | 0.2911 | 0.1472 |
| 0.2824 | 0.1412 | 0.2917 | 0.1443 | — | — | 0.2921 | 0.1458 | 0.2909 | 0.1464 | 0.3025 | 0.1530 |
| 0.3018 | 0.1499 | 0.2981 | 0.1490 | 0.3066 | 0.1537 | 0.2831 | 0.1425 | 0.2879 | 0.1440 | 0.2746 | 0.1418 |
| 0.2875 | 0.1439 | 0.3960 | 0.2004 | 0.4011 | 0.1995 | 0.3925 | 0.1967 | 0.4157 | 0.2074 | — | — |

Dilution Corrected Concentration (mg/mL)

| | | | | | | | | | | | |
|---|---|---|---|---|---|---|---|---|---|---|---|
| — | — | — | — | — | — | 29.53 | 29.73 | 27.86 | 27.64 | 29.11 | 29.45 |
| 28.24 | 28.24 | 29.17 | 28.86 | — | — | 29.21 | 29.15 | 29.09 | 29.28 | 30.25 | 30.60 |
| 30.18 | 29.97 | 29.81 | 29.79 | 30.66 | 30.73 | 28.31 | 28.51 | 28.79 | 28.79 | 27.46 | 28.35 |
| 28.75 | 28.78 | 39.60 | 40.07 | 40.11 | 39.90 | 39.25 | 39.34 | 41.57 | 41.48 | — | — |

The reconstitution efficiencies of each sample were presented in Table 69. They were calculated by determining the percent of silk fibroin dissolved compared to the theoretical silk fibroin concentration.

TABLE 69
The reconstitution efficiencies of silk fibroin solutions
prepared with various buffers and freezing conditions

| | | Freezing | Reconstitution |
|---|---|---|---|
| Sample | Buffer | condition | efficiency (%) |
| 77-A | 2 mM Histidine | Liquid | 96 |
| | | Nitrogen | |
| 77-B | 2 mM Histidine | −80° C. | 96 |
| 77-C | 10 mM Histidine | Liquid | 97 |
| | | Nitrogen | |
| 77-D | 10 mM Histidine | −80° C. | 97 |
| 77-E | 10 mM Phosphate | Liquid | 101 |
| | Buffer | Nitrogen | |
| 77-F | 10 mM Phosphate | −80° C. | 101 |
| | Buffer | | |
| 77-G | 1% Sucrose, 2 mM | Liquid | 95 |
| | Histidine | Nitrogen | |
| 77-H | 1% Sucrose, 2 mM | −80° C. | 94 |
| | Histidine | | |
| 77-I | 1% Sucrose, 2 mM | Liquid | 97 |
| | Histidine, 6% Silk | Nitrogen | |
| | Fibroin | | |
| 77-J | 1% Sucrose, 2 mM | −80° C. | 98 |
| | Histidine, 6% Silk | | |
| | Fibroin | | |

It was observed that all the samples produced clear reconstituted silk solutions. The lowest reconstitution efficiency was seen with samples 77-G and 77-H, lyophilized with 2 mM histidine, 1% sucrose buffer. All other buffers and freezing conditions lead to high reconstitution efficiencies of greater than or equal to 94%. 10 mM phosphate buffer displayed the highest efficiency of 101%. In addition, there was no drastic difference seen in reconstitution efficiency of silk fibroin when comparing the freezing conditions. However, while qualitatively assessing the solutions, it was seen that samples that were frozen at −80° C. had fewer precipitates of silk as compared to samples frozen using liquid nitrogen.

### Rheological Analysis of Reconstituted Silk Fibroin Solutions

Silk fibroin solutions from each described lyophilization buffer and condition were then diluted to 10% (w/v) silk fibroin. 1500 uL of silk solution from each condition was placed onto the Peltier plate of a Bohlin CVOR 150 rheometer. Samples were analyzed at 25° C. using a 40 mm cone plate geometry and a gap of 0.5 mm. Oscillation parameters were set at 1 Hz frequency and 5% strain, while viscosity parameters were set at a shear rate of 0.25 1/s for 120 seconds. The rheological measurements were shown in Table 70. Average was denoted with “Ave.”, and standard deviation was denoted with “SD”. The viscosity, phase angle, shear storage modulus (G′), and shear loss modulus (G) were measured for each sample.

TABLE 70
Rheological measurements of silk fibroin solutions prepared from silk
fibroin lyophilized with various buffers and freezing methods

| | | | | | Ave. | | | | | |
|---|---|---|---|---|---|---|---|---|---|---|
| | | | Ave. | | Phase | Phase | Ave. | | Ave, | |
| | | | Viscosity | Viscosity | Angle | Angie | G′ | G′ | G″ | G″ |
| Sample | Buffer | Condition | (Pa*s) | SD | (°) | SD | (Pa) | SD | (Pa) | SD |
| 77-A | 2 mM | Liquid | 0.158 | 0.027 | 43.496 | 1.454 | 0.195 | 0.029 | 0.186 | 0.037 |
| | Histidine | Nitrogen | | | | | | | | |
| 77-B | 2 mM | −80 | 0.118 | 0.030 | 64.222 | 8.049 | 0.054 | 0.015 | 0.112 | 0.008 |
| | Histidine | | | | | | | | | |
| 77-C | 10 mM | Liquid | 0.068 | 0.020 | 62.549 | 16.523 | 0.066 | 0.056 | 0.112 | 0.026 |
| | Histidine | Nitrogen | | | | | | | | |
| 77-D | 10 mM | −80 | 0.112 | 0.077 | 68.368 | 1.938 | 0.032 | 0.009 | 0.080 | 0.016 |
| | Histidine | | | | | | | | | |
| 77-E | 10 mM | Liquid | 0.094 | 0.031 | 57.972 | 3.174 | 0.064 | 0.003 | 0.103 | 0.018 |
| | PB | Nitrogen | | | | | | | | |
| 77-F | 10 mM | −80 | 0.144 | 0.060 | 47.878 | 2.758 | 0.178 | 0.010 | 0.198 | 0.030 |
| | PB | | | | | | | | | |
| 77-G | 1% | Liquid | 0.162 | 0.140 | 49.992 | 4.536 | 0.244 | 0.217 | 0.304 | 0.293 |
| | Sucrose, | Nitrogen | | | | | | | | |
| | 2 mM | | | | | | | | | |
| | Histidine | | | | | | | | | |
| 77-H | 1% | −80 | 0.158 | 0.008 | 60.472 | 0.048 | 0.111 | 0.049 | 0.195 | 0.087 |
| | Sucrose, | | | | | | | | | |
| | 2 mM | | | | | | | | | |
| | Histidine | | | | | | | | | |
| 77-1 | 1% | Liquid | 0.164 | 0.019 | 63.324 | 0.193 | 0.127 | 0.028 | 0.252 | 0.058 |
| | Sucrose, | Nitrogen | | | | | | | | |
| | 2 mM | | | | | | | | | |
| | Histidine, | | | | | | | | | |
| | 6% SF | | | | | | | | | |
| 77-J | 1% | −80 | 0.217 | 0.057 | 63.207 | 4.879 | 0.151 | 0.000 | 0.304 | 0.065 |
| | Sucrose, | | | | | | | | | |
| | 2 mM | | | | | | | | | |
| | Histidine, | | | | | | | | | |
| | 6% SF | | | | | | | | | |

Rheological analysis of samples lyophilized with different buffers and freezing conditions provided data on the viscosity and phase angle of silk solutions. Viscosity is a measure of a material's resistance to flow, while phase angle is related to the ratio between G′ (elastic/storage modulus) and G″ (viscous/loss modulus). In Table 70, the average viscosity showed variability in the viscosity of silk solutions, which indicated that the properties of lyophilized silk fibroin were highly dependent on the type of buffer and freezing method used. Viscosity of the solutions slightly increased for some of the samples frozen directly at −80° C. For example, samples with silk fibroin lyophilized with 10 mM histidine buffer, 10 mM phosphate buffer, and 1% Sucrose in 2 mM histidine at 6% silk fibroin (77-D,77-F,77-J respectively) had higher viscosities than samples prepared from silk fibroin lyophilized with the same buffers and frozen with liquid nitrogen (77-C,77-E,77-I respectively). On the contrary, sample 77-B (lyophilized with 2 mM histidine buffer and frozen at −80° C.) had lower viscosity as compared to sample 77-A, which was frozen with liquid nitrogen.

Table 70 showed that the range in average phase angle for silk solutions was minimal and ranged from about 43° to about 68°. This range revealed that the silk fibroin solutions were fluid and that phase angle did not differentiate silk fibroin solution freezing/lyophilization conditions.

### Example 34. Analysis of Hydrogels Prepared from Lyophilized Silk Fibroin with Varying Buffers and Freezing Conditions

Hydrogels were prepared from the silk fibroin lyophilized with the varying buffer and freezing conditions described above. The hydrogels were formulated with a concentration of 3% (w/v) silk fibroin degummed with a 480 mb (Batch 77), 10% (w/v) poloxamer-188 (P188) (Sigma-Aldrich, St. Louis, Mo.), 10% (w/v) celecoxib (CXB) (Cipla, Miami, Fla.), and 0.2% (w/v) polysorbate-80 (Croda, Snaith, UK). The formulation may be described by the name 480 mb; hyd: 3% SFf; 10% CXBf; 10% P188f; 0.2% poly-80f. To prepare the hydrogels, the silk fibroin lyophilized with different buffer and freezing conditions was first reconstituted to generate a 40% (w/v) silk fibroin solution. For samples 77-A through 77-H, 300 mg portions of silk fibroin were each brought up in 498 μL of deionized water with mixing at room temperature for 30 minutes to ensure the dissolution of the silk fibroin. For samples 77-I and 77-J, 470 mg portions of silk fibroin were each brought up in 775 μL of deionized water with mixing at room temperature for 30 minutes to ensure the dissolution of the silk fibroin. For all samples, 300 mg of CXB, 975 μl of 0.62% polysorbate-80, and 1.5 mL 20% poloxamer-188 were added to a 4 mL glass vial. The solution was sonicated until homogeneously suspended. 225 μL of the desired 40% (w/v) silk fibroin solution was then added to the glass vial, which was then gently inverted to mix. The formulation was poured into a 5 mL syringe, capped, and placed at 37° C. on a rotator overnight to induce gelation. After gelation, the hydrogels were stored at 4° C. until use. The hydrogels prepared were described in Table 71, along with the percent reconstitution of silk fibroin in solution calculated as described earlier.

TABLE 71
Descriptions of hydrogels (480 mb; hyd; 3% SFf; 10% CXBf;
10% P188f; 0.2% poly-80f) prepared from silk fibroin lyophilized
with different buffer and freezing conditions

| | | | % |
|---|---|---|---|
| | | | Reconstitution |
| | | | from solution |
| | | | used to prepare |
| Sample | Buffer | Condition | hydrogel |
| 77-A-h | 2 mM Histidine | Liquid | 96% |
| | | Nitrogen | |
| 77-B-h | 2 mM Histidine | −80 | 96% |
| 77-C-h | 10 mM Histidine | Liquid | 97% |
| | | Nitrogen | |
| 77-D-h | 10 mM Histidine | −80 | 97% |
| 77-E-h | 10 mM Phosphate | Liquid | 101% |
| | Buffer | Nitrogen | |
| 77-F-h | 10 mM Phosphate | −80 | 101% |
| | Buffer | | |
| 77-G-h | 1% Sucrose, 2 mM | Liquid | 95% |
| | Histidine | Nitrogen | |
| 77-H-h | 1% Sucrose, 2 mM | −80 | 94% |
| | Histidine | | |
| 77-I-h | 1% Sucrose, 2 mM | Liquid | 103% |
| | Histidine, 6% silk | Nitrogen | |
| | fibroin | | |
| 77-J-h | 1% Sucrose, 2 mM | −80 | 105% |
| | Histidine, 6% silk | | |
| | fibroin | | |

Analysis of the Rheological Properties of Hydrogels Prepared from Silk Fibroin Lyophilized with Different Buffer and Freezing Conditions

To analyze the rheology of the hydrogels, 600 μL of each hydrogel sample was loaded onto the Peltier plate of a Bholin CVOR 150 rheometer. Samples were analyzed at 25° C. using a 20 mm parallel plate and a gap of 1.0 mm. Oscillation parameters were set at a frequency of 1 Hz and 0.01% strain for 146 seconds with 15 samples. Viscosity was measured at a shear rate of 1 1/s for 135 seconds with 15 samples. The results of the rheological experiments were presented in Table 72. The viscosity, phase angle, shear storage modulus (G′), and shear loss modulus (G″) were measured for each sample.

TABLE 72
The rheological properties of hydrogels (480 mb; hyd; 3% SFf; 10% CXBf; 10% P188f; 0.2% poly-
80f) prepared from silk fibroin lyophilized under varying buffer and freezing conditions

| | | Viscosity | Phase | Phase | | G′ | | G″ |
|---|---|---|---|---|---|---|---|---|
| Hydrogel | Viscosity | Standard | Angle | Angle | G′ | Standard | G″ | Standard |
| Sample | (Pa*s) | Deviation | (°) | SD | (Pa) | Deviation | (Pa) | Deviation |
| 77-A-h | 117.91 | 4.54 | 15.96 | 0.29 | 11954.60 | 1167.64 | 3420.21 | 345.961 |
| 77-B-h | 80.10 | 4.33 | 15.22 | 0.56 | 7158.56 | 697.81 | 1951.16 | 232.434 |
| 77-C-h | 51.44 | 1.67 | 14.51 | 0.79 | 5675.25 | 773.41 | 1464.14 | 181.907 |
| 77-D-h | 243.27 | 8.75 | 13.04 | 0.41 | 11930.28 | 1026.73 | 2766.93 | 286.623 |
| 77-E-h | 96.52 | 4.93 | 13.76 | 2.01 | 9209.08 | 928.31 | 2248.78 | 322.209 |
| 77-F-h | 96.50 | 2.65 | 12.76 | 0.66 | 8591.34 | 806.60 | 1947.98 | 232.117 |
| 77-G-h | 159.57 | 6.29 | 13.25 | 2.54 | 9853.04 | 1234.62 | 2280.70 | 406.044 |
| 77-H-h | 87.41 | 3.26 | 14.83 | 0.57 | 5706.29 | 540.05 | 1513.35 | 182.463 |
| 77-I-h | 56.54 | 4.28 | 19.94 | 4.32 | 5018.77 | 828.53 | 1782.34 | 237.631 |
| 77-J-h | 189.11 | 8.21 | 12.16 | 0.24 | 9260.47 | 688.47 | 1995.67 | 161.012 |

Table 72 showed that the viscosity for hydrogels was higher (50 to 250 Pa*s) than the viscosity of the corresponding silk solutions (0.05 to 0.200 Pa*s) seen in Table 70. In addition, the viscosity of the hydrogels showed similar trends to the silk fibroin solutions from which they were prepared. As seen for the corresponding silk fibroin solutions, the viscosity of the silk fibroin hydrogels was variable between samples. Hydrogels that were prepared from silk fibroin lyophilized in either 10 mM histidine buffer or 1% sucrose with 2 mM histidine and 6% silk fibroin, that were also frozen at −80° C., had higher viscosities than their hydrogel counterparts that were frozen with liquid nitrogen. The viscosity of hydrogels prepared from silk fibroin in 10 mM phosphate buffer showed differing viscosity as solutions, but the same viscosity at hydrogels (77-E-h, 77-F-h). Table 72 also showed a minimal difference in the phase angle for silk hydrogels (15°-20°). These data displayed the solid, gel-like state of the resulting hydrogels, but it showed that phase angle could not be used to differentiate between samples. The reconstitution efficiency of silk fibroin prepared in phosphate buffer, combined with the consistent viscosities between hydrogels prepared from said silk fibroin lyophilized in phosphate buffer (regardless of freezing technique), rendered the lyophilization of silk fibroin in phosphate buffer the optimal condition.

Analysis of the Injectability of Hydrogels Prepared from Silk Fibroin Lyophilized with Different Buffer and Freezing Conditions

Injection force experiments were conducted with a Mark-10 M5-100 Force gauge attached to a Mark-10 motorized test stand (MKESM303). Hydrogel samples were mixed to ensure homogeneity before being loaded into 1 mL syringes and capped with 27G, ½″ needles. The syringe for each sample was then inserted into a Mark-10 syringe compression fixture. The test stand was set to compress the syringe plunger and extrude the hydrogel at a rate of 0.5 in/min (0.2 mL/min). Force data was collected over 60 seconds (20 points per second) and exported and graphed to determine the injectability force plateau. Each sample was injected in duplicate and average (Avg) injection force over the plateau was calculated. The results of the experiments were presented in Table 73. The experiments were performed in duplicate, and the results were averaged together.

TABLE 73
The average injection forces of the hydrogels (480 mb; hyd; 3%
SFf; 10% CXBf; 10% P188f; 0.2% poly-80f) prepared from silk fibroin
lyophilized with various buffers and freezing conditions

| | | Sample 1 | Sample | Sample 2 | Combined | Combined |
|---|---|---|---|---|---|---|
| | Sample 1 | Standard | 2 Avg | Standard | Average | Standard |
| Samples | Avg (N) | Deviation | (N) | Deviation | (N) | Deviation |
| 77-A-h | 5.35 | 0.26 | 6.29 | 0.13 | 5.82 | 0.089 |
| 77-B-h | 7.25 | 0.17 | 7.44 | 0.25 | 7.34 | 0.053 |
| 77-C-h | 5.72 | 0.20 | 6.50 | 0.17 | 6.11 | 0.022 |
| 77-D-h | 9.53 | 0.22 | 9.46 | 0.20 | 9.50 | 0.020 |
| 77-E-h | 5.36 | 0.35 | 6.28 | 0.14 | 5.82 | 0.148 |
| 77-F-h | 4.32 | 0.20 | 5.32 | 0.35 | 4.82 | 0.109 |
| 77-G-h | 7.37 | 0.28 | 7.94 | 0.41 | 7.66 | 0.096 |
| 77-H-h | 5.69 | 0.20 | 6.12 | 0.15 | 5.91 | 0.038 |
| 77-I-h | 4.12 | 0.26 | 5.44 | 0.17 | 4,78 | 0.064 |
| 77-J-h | 8.57 | 0.23 | 9.17 | 0.35 | 8.87 | 0.080 |

Table 73 showed that the injection force for hydrogels made from silk lyophilized in different conditions ranged from 4 to 9 N when using a 1 mL syringe, and 27 G, ½″ needle at a rate of 0.2 mL/min. The hydrogels with the 3 highest injection forces (77-D-h, 77-J-h, and 77-G-h) were the samples that displayed the highest viscosities in Table 72. These were the samples which contained 10 mM histidine buffer (frozen at −80° C.), 1% sucrose and 2 mM histidine buffer with 6% SF (frozen at −80° C.), and 1% sucrose in 2 mM histidine buffer (frozen in liquid nitrogen). The remaining samples did not show trends that were represented by the viscosity of the formulations. In general, there were no major differences in the injectability of hydrogels prepared from silk fibroin lyophilized with various buffer and freezing conditions.

### Example 35. Effects of Membrane Size on the Dialysis of Silk Fibroin

This study was conducted to optimize the molecular weight cutoff (MWCO) of the membranes used during dialysis of silk fibroin. Fully processed silk fibroin solutions and hydrogels were characterized via reconstitution efficiency, rheology, and injectability.

Silk yarn (Jiangsu SOHO Silk and Textile Co.) was degummed at 100° C. for 480 minutes in 0.02 M sodium carbonate solution (sodium carbonate was purchased from Fisher Bioreagents), followed by three warm (65° C.) and three room temperature (RT) washes in MilliQ® water. The procedure went as described herein. 2 L of deionized water was heated in a 4 L glass beaker covered with aluminum foil. 4.24 g of sodium carbonate (or 2.12 g per liter) was added to the water until it fully dissolved. A Thermocouple thermometer was used to monitor the temperature of the water. Once water reached a steady boil, 20 g of silk yarn was weighed and added. A serological pipette was used to completely disperse the silk. The silk was boiled for 4 hours (240 minutes). After completion of boil, the degummed silk was briefly rinsed in cold water to get rid of any remaining sodium carbonate, and it was placed in 4 L of clean deionized water at 4° C. overnight. The following day, the silk was boiled for an additional 4 hours in the sodium carbonate buffer. After completion of the boil (480 minutes total), the silk was directly transferred into a beaker with 2 L of warm deionized water between 60° C.-70° C. for 20 minutes. This step was repeated twice. (3 rinses in total, 20 minutes each). After the last warm water wash, the silk was directly transferred into a beaker with 4 L of cold deionized water for 20 minutes. This step was repeated twice. (3 rinses in total, 20 minutes each) After the washes, the silk was wringed in order to expel out all of the water. The silk was then pulled apart, removing any large clumps. The pulled apart silk was then placed on aluminum foil in the fume hood overnight, and covered in Steri-wrap® for drying.

The resulting silk fibroin was dried overnight at room temperature (RT), weighed, and dissolved at 20% (w/v) in 9.3 M lithium bromide (LiBr) solution (lithium bromide was purchased from Fisher Chemical, Waltham Mass.) for five hours at 60° C. The procedure went as follows. The dried silk was weighed to be 11.84 g. 55 mL of stock 9.3 M LiBr solution was made by weighing 44.44 g of LiBr and adding it slowly to 33 mL of DI water. Using a measuring cylinder, the total volume of the solution was brought to 55 mL with DI water. The lithium bromide solution was then filtered through a 0.22 μm PES vacuum filtration unit. The dried silk was tightly pushed into the bottom of a 100 mL beaker and 47.3 ml of the filtered LiBr solution was added to beaker, ensuring that the silk was completely submerged. The beaker was placed at 60° C. for 4 hours until a clear, yellow solution was obtained, and the silk was completely dissolved.

This solution was dialyzed against MilliQ water in pre-wetted 50 kDa (Spectra/Por, CAS: 131384, Lot: 3282822) or 3.5 kDa (Spectra/Por, CAS: 132552T, Lot: 3268482) regenerated cellulose membrane for 48 hours at 4° C. with 6 water exchanges. The dialysis went as follows. The 50 kDa and dry 3.5 kDa cellulose tubing was cut and placed in deionized water prior to transferring silk solution for 20 minutes to rinse. 30 ml of the 480 mb silk solution was added to each of the 50 kDa and 3.5 kDa dialysis tubing. Silk was dialyzed against 5 L of DI water on a stir plate at 4° C. The water was changed 6 times change over a period of 48 hours. Conductivity was measured using a digital probe after the last water change to ensure the completion of dialysis.

The final solution was centrifuged for 20 minutes at 3,900 RPM (on an Eppendorf tabletop refrigerated centrifuge) and 4° C. to remove insoluble particles. The procedure went as follows. After dialysis was completed, the dialysis tubing was removed, and the silk solution was poured into two different 100 ml beakers labeled A for silk solution from 3.5 kDa tubing and B for silk solution from 50 kDa tubing. The volume of silk solution from 3.5 kDa tubing and 50 kDa tubing was measured to be 74 mL and 58 mL, respectively. The tubes were spun at 3,900 RPM (on an Eppendorf tabletop refrigerated centrifuge) for 20 minutes at 4° C. The supernatant was collected.

The concentration of the resulting solution was then determined using a UV absorbance assay (280 nm). Briefly, standards of concentrations 0.5%, 0.25%, 0.125%, 0.0625%, 0.03125% and blank (5, 2.5, 1.25, 0.625, 0.3125 and 0 mg/ml) were made from a pre-measured 5% silk solution for A280 reading. An aliquot of the silk solutions was then diluted 1:20 and 1:40 using 1×PBS Buffer and measured against the standard curve at 280 nm absorbance to determine concentration. The appropriate amount of phosphate buffer (potassium phosphate monobasic and potassium phosphate dibasic were purchased from SAFC, St. Louis Mo.) was then added, and mixed thoroughly without forming bubbles, to obtain a final concentration of 30 mg/ml SF. Final 30 mg/mL solutions were filtered through a 0.2 μm PES membrane prior to aliquoting and freezing. The resulting dialyzed solutions of silk fibroin were described in Table 74.

TABLE 74
Summary of the different dialysis conditions
for silk fibroin extraction

| | | | Volume | Final Volume |
|---|---|---|---|---|
| | Dialysis Membrane | Concentration | Post | of 3% w/v |
| | (Regenerated | Post Dialysis | Dialysis | silk fibroin |
| Sample | Cellulose) | (w/v %) | (mL) | solution (mL) |
| 78-A | 3.5 kDa MWCO; | 8.23 | 74 | 202.96 |
| | 10 mM phosphate | | | |
| | buffer | | | |
| 78-B | 50 kDa MWCO; | 8.60 | 58 | 166.24 |
| | 10 mM phosphate | | | |
| | buffer | | | |

Under aseptic conditions, filtered solutions were aliquoted into 50 mL conical tubes (10 mL per tube), covered with Steri-Wrap®, and frozen by placing directly in the −80° C. freezer overnight. All tubes were lyophilized in a manifold freeze dryer (Labconco FreeZone 4.5) for 60-72 hours

### Rheological Analysis of Reconstituted Silk Fibroin Solutions

Silk solutions from each dialysis condition were then diluted to 10% (w/v) silk fibroin with MilliQ® water. 1.5 mL of silk solution from each dialysis condition was placed onto the Peltier plate of a Bohlin CVOR 150 rheometer. Samples were analyzed at 25° C. using a 40 mm cone plate geometry and a gap of 0.5 mm. Oscillation parameters were set at 1 Hz frequency and 5% strain, while the viscosity parameter was set at a shear rate of 0.25 1/s and measured for 120 seconds. The results of the rheological analyses were presented in Table 75. The viscosity, phase angle, elastic modulus (G′), and viscous modulus (G″) were measured for each sample. Standard deviations were represented with “SD”.

TABLE 75
Rheological measurements of silk fibroin solutions prepared
from silk fibroin dialyzed in membranes of various MWCO's

| | | | | | Phase | Phase | | | | |
|---|---|---|---|---|---|---|---|---|---|---|
| | | | Viscosity | Viscosity | Angle | Angle | G′ | G′ | G″ | G″ |
| Sample | Buffer | Condition | (Pa*s) | SD | (°) | SD | (Pa) | SD | (Pa) | SD |
| 78-A | 10 mM | 3.5 kDa | 0.145 | 0.014 | 37.71 | 2.50 | 0.27 | 0.02 | 0.20 | 0.01 |
| | PR | Membrane | | | | | | | | |
| 78-B | 10 mM | 50 kDa | 0.149 | 0.018 | 53.88 | 2.51 | 0.20 | 0.04 | 0.28 | 0.04 |
| | PB | Membrane | | | | | | | | |

Rheological analysis of samples dialyzed using different membranes provided insight into viscosity and phase angle of silk solutions and resulting hydrogels. Viscosity is a measure of a material's resistance to flow while phase angle is related to the ratio between G′ (elastic/storage modulus) and G″ (viscous/loss modulus). Table 75 showed that the viscosity values for silk solutions from both dialysis conditions (3.5 kDa or 50 kDa MWCO membranes) were similar. This indicated that the type of membrane used in dialysis did not significantly impact the viscosity of silk fibroin solution after reconstitution. However, differences were observed in the phase angle of the silk fibroin solutions. Silk fibroin solutions dialyzed with 50 kDa membrane had a higher phase angle (53°) as compared to the 3.5 kDa membrane (37°). The phase angle difference highlighted the more fluid, viscous nature of silk solutions dialyzed with 50 kDa membrane as opposed to the stiffer properties of silk solution dialyzed with 3.5 kDa membrane.

### Example 36. Analysis of Hydrogels Prepared from Silk Fibroin Dialyzed in Differing Dialysis Membranes

Hydrogels were prepared from the silk fibroin dialyzed in dialysis membranes of varying molecular weight cutoff (MWCO). The hydrogels were formulated with a concentration of 3% (w/v) silk fibroin degummed with a 480 mb (Batch 78), 10% (w/v) poloxamer-188 (P188) (Sigma-Aldrich, St. Louis, Mo.), 101% (w/v) celecoxib (CXB) (Cipla, Miami, Fla.), and 0.2% (w/v) polysorbate-80 (Croda, Snaith. UK). The formulation may be described by the name 480 mb; hyd; 3% SFf; 10% CXBf; 10% P188f; 0.2% poly-80f. To prepare the hydrogels, the lyophilized silk fibroin (that had been prepared via the dialysis with either a 3.5 kDa or a 50 kDa MWCO membrane, as described above), was reconstituted into a 40% (w/v) solution by adding 498 μL of DI water to 300 mg samples of silk fibroin. The silk fibroin was then mixed for 30 minutes at room temperature to ensure the dissolution of the silk fibroin. In a 4 mL glass vial, 300 mg of CXB, 975 μL of 0.62% polysorbate-80, and 1.5 mL 20% poloxamer-188 were added. The solution was sonicated until the celecoxib was homogeneously suspended. 225 μL of the 40% (w/v) silk fibroin solution was added to the glass vial and gently inverted to mix. The suspension was poured into a 5 mL syringe, capped and placed at 37° C. on a rotator for 16 hours (overnight) to induce gelation. The resulting hydrogels were stored at 4° C. until use. The hydrogels prepared were described in Table 76.

TABLE 76
Descriptions of hydrogels (480 mb; hyd; 3% SFf; 10%
CXBf; 10% P188f; 0.2% poly-80f) prepared from silk
fibroin dialyzed with different MWCO membranes

| | Dialysis Membrane | Silk fibroin | Silk fibroin | |
|---|---|---|---|---|
| | (Regenerated | boil time | concentration | |
| Sample | Cellulose) | (mb) | (%) | Excipient |
| 78-A-h | 3.5 kDa MWCO | 480 | 3 | 10% P188 |
| 78-B-h | 50 kDa MWCO | 480 | 3 | 10% P188 |

Analysis of the Rheological Properties of Hydrogels Prepared from Silk Fibroin Dialyzed with Different MWCO Membranes

600 μL of each hydrogel sample was loaded onto the Peltier plate of a Bholin CVOR 150 rheometer. Samples were analyzed at 25° C. using a 20 mm parallel plate spindle and a gap of 1 mm. Oscillation parameters were set at 1 Hz frequency and 0.01% strain. The viscosity parameter was measured at shear rate of 1 1/s for over 135 seconds. The results of the rheological experiments were presented in Table 77. The viscosity, phase angle, elastic modulus (G′), and viscous modulus (G″) were measured for each sample. “SD” denoted standard deviation.

TABLE 77
The rheological properties of hydrogels (480 mb; hyd; 3% SFf; 10% CXBf; 10% P188f;
0.2% poly-80f) prepared from silk fibroin dialyzed with varying MWCO membranes

| | | | Phase | Phase | | | | |
|---|---|---|---|---|---|---|---|---|
| | Viscosity | Viscosity | Angle | Angle | | | | |
| Sample | (Pa*s) | SD | (°) | SD | G′ (Pa) | G′ SD | G″ (Pa) | G″ SD |
| 78-A-h | 180.78 | 22.42 | 12.47 | 0.51 | 12023.47 | 1140.19 | 2652.88 | 209.40 |
| 78-B-h | 199.75 | 5.96 | 13.41 | 0.62 | 14811.47 | 1387.69 | 3536.25 | 421.68 |

Rheologic characterization of hydrogels prepared from the silk fibroin solutions dialyzed with different membranes had similar trends to the those of the solutions alone. Table 77 showed that the viscosities for the two hydrogel formulations were similar. As expected, these values were higher (150 to 200 Pa*s) than the viscosity of the corresponding silk fibroin solutions (approximately 0.15 Pa*s, as seen in Table 75). Phase angle measurements showed that hydrogels made from silk dialyzed with 3.5 kDa membrane exhibit slightly stiffer, gel-like material than hydrogels prepared from silk dialyzed with 50 kDa membrane. This result may be due to the molecular weight of the silk fibroin. The lower MWCO membrane (3.5 kDa) will retain a lower molecular weight than the higher MWCO membrane (50 kDa). These lower molecular weight fragments may contribute to tighter silk fibroin networks, resulting in stiffer solutions and gels

Analysis of the Injectability of Hydrogels Prepared from Silk Fibroin Dialyzed with Different MWCO Membranes

Injection force experiments were conducted with a Mark-10 M5-100 Force gauge attached to a Mark-10 motorized test stand (MKESM303). Hydrogel samples were mixed back and forth between 2 syringes to ensure homogeneity before being loaded into 1 mL syringes and capped with 27G, ½″ needles. The syringe for each sample was then inserted into a Mark-10 syringe compression fixture, and the test stand was set to move the head onto the syringe plunger and extrude the hydrogel at a rate of 0.5 in/min (0.2 mL/min). Data was collected over 60 seconds (20 points per second), exported, and graphed to find the injectability force plateau. The average value was taken over this plateau region. Each sample was injected in triplicate and average injection force was calculated. The results of the injectability experiments were presented in Table 78.

TABLE 78
The average injection forces of the hydrogels (480 mb; hyd; 3% SFf; 10% CXBf; 10% P188f;
0.2% poly-80f) prepared from silk fibroin dialyzed with different MWCO membranes

| | Sample 1 | | Sample 2 | | Sample 3 | | Combined | |
|---|---|---|---|---|---|---|---|---|
| | Average | Standard | Average | Standard | Average | Standard | Average | Standard |
| Samples | (N) | Deviation | (N) | Deviation | (N) | Deviation | (N) | Deviation |
| 77-A-h | 10.16 | 0.26 | 9.10 | 0.18 | 10.64 | 0.25 | 9.97 | 0.044 |
| 77-B-h | 9.82 | 0.27 | 8.67 | 0.17 | 9.89 | 0.28 | 9.46 | 0.063 |

Table 78 showed that the injection force for hydrogels made from silk dialyzed in different conditions ranged from 8 to 10 N. Hydrogels made from silk dialyzed with a 3.5 kDa membrane (78-A-h) were slightly more difficult to inject as compared to hydrogels made from silk dialyzed with 50 kDa membrane (78-B-h). This difference coincided with the phase angle measurements, which showed that that the hydrogels made from silk dialyzed with a 3.5 kDa membrane were the stiffer hydrogels. Injection force data demonstrated that these stiffer gels took more force to inject. Therefore, the use of the 50 kDa membrane lead to the preparation of silk fibroin solutions with a narrower molecular weight range that exhibited more fluid-like properties. This membrane has been selected for use in the silk fibroin extraction process.

### Example 37. Preparation of Fluorescein Isothiocyanate (FITC)-Labeled Silk Fibroin (FITC-SF) Solution

Silk fibroin (SF) was labeled with fluorescein isothiocyanate (FITC). 420 mg of sodium bicarbonate (Spectrum: cat #SO125; Lot #2BF0355) was dissolved in 9 mL of deionized (DI) water. The pH was adjusted to 9.0 using 1N NaOH/HCl. A quantity of DI water sufficient to raise the volume to 10 mL was added to prepare a 0.5M sodium bicarbonate solution.

1.5M hydroxylamine was prepared fresh by dissolving 262 mg hydroxylamine in 2.0 mL of water. The pH was adjusted to 8.5 using 10N NaOH, and a quantity of DI water sufficient to raise the volume to 2.5 mL.

Immediately before performing the labeling reaction, FITC (three 10 mg vials, ThermoFisher) was dissolved in 0.5 mL of DMSO resulting in a 20 mg/mL solution of FITC in DMSO.

A 5% silk fibroin solution (480 mb; Batch 88) containing 50 mM sucrose was thawed, and 4 mL of the solution was moved to a 20 mL scintillation vial. 1 mL 500 mM sodium bicarbonate buffer was added to the vial containing the silk fibroin solution. If needed, the pH was adjusted to between 8.5-9.0 using 1N NaOH. A sample of the silk fibroin solution was retained as a control.

All buffers and solutions were filtered through 0.2 μm filters under aseptic conditions with the exception of the silk fibroin solution and the FITC in DSMO solution.

The labeling reaction was performed by adding 1.44 mL FITC in DMSO to 4.5 mL of the silk fibroin solution in a 20 mL glass vial. The vial was kept out of light and incubated at room temperature (RT) for 2 hours on a rocker resulting in 3×FITC-labeled silk fibroin (FITC-SF).

The control sample of silk fibroin solution was prepared by adding 1.4 mL of DMSO to 4.5 mL of the silk fibroin solution in a 20 mL glass vial. The mixture was incubated at RT for 2 hours on a rocker.

After the two-hour incubation, 0.6 mL hydroxylamine solution was added to each reaction, and the mixture was incubated again at RT for one hour on a rocker. The pH was then adjusted to 7.0 using 1N HCl. Each solution was transferred to separate 20 kDa dialysis cassettes. Each solution was protected from light while dialyzed for four times in 4.5 L exchanges at 4° C. over 72 hours. Dialyzed solutions were filtered under aseptic conditions through a 0.2 μm polyethersulfone (PES) filter unit. Final solutions were stored in sterile container at 4° C. until use.

### Example 38. Confirmation of the Conjugation of FITC to Silk Fibroin

High performance liquid chromatography (HPLC) may be used to confirm conjugation is successful in the FITC-labeled silk fibroin (FITC-SF) solution. An Agilent 1260 BioInert HPLC system equipped with a Waters X-Bridge Protein BEH SEC, 200 Å, 3.5 μm column may be used. An isocratic flow of mobile phase (100 mM Tris-HCl with 400 mM sodium perchlorate, pH 8.5) at 0.86 mL/min may be used to elute analytes. Successful FITC labelling of SF 480 mb will be determined by monitoring protein absorbance at 280 nm and FITC emission at 525 nm following excitation at 490 nm. Samples may be diluted to 1% (w/v) prior to injection. The data may show overlapping UV and fluorescence profiles for the silk fibroin and FITC-SF, which would represent successful conjugation since the molecular weight of FITC is smaller than the silk fibroin (400 Da vs. >6 kDa).

1. A silk-based product (SBP) for use in a therapeutic application, an agricultural application, and/or a material science application, wherein the SBP comprises processed silk, wherein the processed silk comprises or is derived from one or more articles, said one or more articles is selected from the group consisting of raw silk, silk fiber, silk fibroin, and a silk fibroin fragment.

2. The SBP of claim 1 for use in a therapeutic application, wherein the SBP comprises or is combined with one or more articles selected from the group consisting of:
a. a pharmaceutical composition, the pharmaceutical composition optionally comprising one or more of:
i. an excipient, wherein the excipient comprises one or more members selected from the group consisting of any of those listed in Table 1; and
ii. a therapeutic agent, wherein the therapeutic agent comprises one or more members selected from the group consisting of any of those listed in Table 3;
b. an implant, the implant optionally comprising one or more of:
i. an excipient, wherein the excipient comprises one or more members selected from the group consisting of any of those listed in Table 1;
ii. a therapeutic agent, wherein the therapeutic agent comprises one or more members selected from the group consisting of any of those listed in Table 3;
iii. a coating;
iv. a gel or hydrogel;
v. a scaffold;
vi. a particle; and
vii. a device, wherein the device comprises one or more members selected from the group consisting of any of those listed in Table 6;
c. a coating, the coating optionally comprising one or more of:
i. an excipient, wherein the excipient comprises one or more members selected from the group consisting of any of those listed in Table 1; and
ii. a therapeutic agent, wherein the therapeutic agent comprises one or more members selected from the group consisting of any of those listed in Table 3;
d. a food or health supplement; and
e. a device, the device optionally comprising one or more of:
i. a synthetic material; and
ii. a therapeutic agent, wherein the therapeutic agent comprises one or more members selected from the group consisting of any of those listed in Table 3.

3. The SBP of claim 1 for use in an agricultural application, wherein the SBP comprises or is combined with one or more members selected from the group consisting of:
a. an agricultural composition, wherein the agricultural composition optionally comprises one or more members selected from the group consisting of:
i. a cargo, wherein the cargo comprises one or more members selected from the group consisting of any of those listed in Table 7;
ii. a coating;
iii. a fertilizer;
iv. a nutrient, wherein the nutrient comprises one or more members selected from the group consisting of any of those listed in Table 7;
v. an agricultural product;
vi. a pest control agent, wherein the pest control agent optionally comprises a pesticide selected from one or more members of the group consisting of:
1. a parasiticide, wherein the parasiticide comprises one or more members selected from the group consisting of any of those listed in Table 7;
2. an insecticide, wherein the insecticide comprises one or more members selected from the group consisting of any of those listed in Table 7;
3. an herbicide, wherein the herbicide comprises one or more members selected from the group consisting of any of those listed in Table 7; and
4. an anti-fungal or fungicide, wherein the anti-fungal or fungicide comprise one or more members selected from the group consisting of any of those listed in Table 7;
vii. a soil stabilizer comprising one or more members selected from the group consisting of any of those listed in Table 7;
viii. a biological system comprising at least one microbe and/or probiotic; and
ix. an agricultural therapeutic agent comprising one or more members selected from the group consisting of any of those listed in Table 3 and any of those listed in Table 7; and
b. an agricultural device, wherein the agricultural device optionally comprises one or more members selected from the group consisting of:
i. an article of agricultural equipment;
ii. a crop storage device;
iii. a landscaping fabric; and
iv. a pest control device.

4. The SBP of claim 1 for use in a material science application, wherein the SBP comprises or is combined with a material, wherein the material comprises one or more articles selected from the group consisting of:
a. an adhesive;
b. a biomaterial;
c. a coating;
d. a conductor;
e. a composting agent;
f. a cosmetic, the cosmetic optionally comprising one or more members selected from the group consisting of any of those listed in Table 9;
g. an emulsifier;
h. an excipient, the excipient optionally comprising one or more members selected from the group consisting of any of those listed in Table 1;
i. a fiber;
j. a film;
k. a filter;
l. a food product or additive;
m. an insulator;
n. a lubricant;
o. a membrane;
p. a metal or metal replacement;
q. a microneedle;
r. a nanomaterial;
s. a particle;
t. a paper additive;
u. a plastic or plastic replacement;
v. a polymer;
w. a sensor;
x. a textile; and
y. a thickening agent.

5-8. (canceled)

9. The SBP of claim 1, wherein the processed silk comprises silk fibroin, wherein the silk fibroin comprises a plurality of silk fibroin fragments, and wherein each of the plurality of silk fibroin fragments comprises a molecular weight of from about 1 kDa to about 350 kDa.

10-23. (canceled)

24. The SBP of claim 1, wherein the processed silk comprises or is included in one or more members selected from the group consisting of yarn, thread, string, a nanofiber, a particle, a nanoparticle, a microsphere, a nanosphere, a powder, a solution, a gel, a hydrogel, an organogel, a mat, a film, a foam, a membrane, a rod, a tube, a patch, a sponge, a scaffold, a capsule, an excipient, an implant, a solid, a coating, and a graft.

25-28. (canceled)

29. The SBP of claim 2, wherein the therapeutic application comprises one or more members selected from the group consisting of:
a. treatment, prevention, mitigation, alleviation, and/or curing of a disease, disorder, and/or condition in a subject;
b. promotion of health, nutrition, and/or wellbeing in a subject;
c. support or promotion of reproduction in a subject;
d. preparation of a therapeutic device; and
e. diagnosis of a disease, disorder, and/or condition in a subject.

30-32. (canceled)

33. The SBP of claim 29, wherein the biological agent comprises one or more members selected from the group consisting of a macromolecule, a carbohydrate, a peptide, a protein, a nucleic acid, a virus, a virus particle, a vesicle, a cell, a spore, a bacteria, and a tissue.

34-39. (canceled)

40. The SBP of claim 29, wherein the SBP comprises a therapeutic agent, wherein the therapeutic agent comprises one or more members selected from the group consisting of:
a. an analgesic agent, wherein the analgesic agent comprises one or more members selected from the group consisting of any of those listed in Table 3;
b. an anesthetic agent;
c. an antianxiety medication;
d. an antibacterial agent, wherein the antibacterial agent comprises one or more members selected from the group consisting of any of those listed in Table 3;
e. an antibody, wherein the antibody comprises one or more members selected from the group consisting of any of those listed in Table 3;
f. an antidepressant;
g. an anti-emetic agent;
h. an antifungal agent, wherein the antifungal agent comprises one or more members selected from the group consisting of any of those listed in Table 3;
i. an antigen, wherein the antigen comprises one or more members selected from the group consisting of any of those listed in Table 3;
j. an anti-inflammatory agent, wherein the anti-inflammatory agent comprises one or more members selected from the group consisting of any of those listed in Table 3;
k. an antimalarial agent, wherein the antimalarial agent comprises one or more members selected from the group consisting of any of those listed in Table 3;
l. an antiparasitic agent;
m. an antipsychotic agent;
n. an antipyretic agent, wherein the antipyretic agent is selected from the group consisting of choline salicylate, magnesium salicylate, metamizole, nimesulide, phenazone, salicylate, and sodium salicylate;
o. an antiseptic agent, wherein the antiseptic agent comprises one or more members selected from the group consisting of any of those listed in Table 3;
p. an antiviral agent;
q. a blood thinner;
r. a chemotherapeutic agent;
s. a contrasting agent;
t. a cytokine, wherein the cytokine comprises one or more members selected from the group consisting of any of those listed in Table 3;
u. an herbal preparation, wherein the herbal preparation comprises one or more members selected from the group consisting of any of those listed in Table 3;
v. a health supplement, wherein the health supplement comprises one or more members selected from the group consisting of any of those listed in Table 3;
w. a hemostatic agent;
x. a hormone, wherein the hormone comprises one or more members selected from the group consisting of any of those listed in Table 3;
y. an imaging agent;
z. an inhalant or respiratory agent;
aa. a motility or anti-motility agent;
bb. a non-steroidal anti-inflammatory drug (NSAID), wherein the NSAID comprises one or more members selected from the group consisting of any of those listed in Table 3;
cc. an oxidant and/or antioxidant, wherein the oxidant and/or antioxidant comprises one or more members selected from the group consisting of any of those listed in Table 3;
dd. a peptide, wherein the peptide comprises one or more members selected from the group consisting of any of those listed in Table 3;
ee. a smoking cessative agent;
ff. a statin, wherein the statin comprises one or more members selected from the group consisting of any of those listed in Table 3;
gg. a stimulant, wherein the stimulant comprises one or more members selected from the group consisting of any of those listed in Table 3;
hh. a targeted cancer therapy drug;
ii. a tranquilizer, wherein the tranquilizer comprises one or more members selected from the group consisting of any of those listed in Table 3;
jj. a wound healing agent; and
kk. an ion, metal, and/or mineral, wherein the ion, metal, and/or mineral are selected from the group consisting of any of those listed in Table 3.

41-45. (canceled)

46. The SBP of claim 3, wherein the agricultural application comprises one or more members selected from the group consisting of:
a. farming;
b. plant growth, yield, reproduction, and/or health;
c. preparing and/or applying soil and/or mulch;
d. weed control;
e. pest control;
f. disease control;
g. seed treatment;
h. seed storage;
i. animal growth, yield, reproduction, and/or health;
j. agricultural product preservation and/or treatment; and
k. controlling access to water, air, and/or sunlight.

47. The SBP of claim 46, wherein the SBP comprises an agricultural composition, wherein the agricultural composition is formulated for application to one or more members selected from the group consisting of:
a. a plant or plant product;
b. a seed;
c. a planting substrate, wherein the planting substrate comprises one or more members selected from the group consisting of soil, mulch, sand, rocks, a sponge, a gel, a matrix, and a mesh;
d. a weed;
e. a pest, a pest habitat, and/or a pest-susceptible surface;
f. a fertilizer; and
g. a device.

48-56. (canceled)

57. The SBP of claim 47, wherein the agricultural composition comprises a coating, wherein the coating is used for one or more purposes selected from the group consisting of:
a. protection of a seed, plant, planting substrate, agricultural product, or device;
b. fertilizing and/or promoting germination of a coated seed or plant;
c. encasing a payload;
d. delivering a payload;
e. modulating nutrient and/or water uptake;
f. stabilizing a payload; and
g. controlling the release of a payload.

58. (canceled)

59. The SBP of claim 57, wherein the agricultural composition comprises a coating agent, and wherein the coating agent comprises one or more compounds selected from the group consisting of polyethylene glycol, methylcellulose, hypromellose, ethylcellulose, gelatin, hydroxypropyl cellulose, titanium dioxide, zein, poly(alkyl)(meth)acrylate, and poly(ethylene-co-vinyl acetate).

60. The SBP of claim 57, wherein the agricultural composition comprises a coated seed.

61-68. (canceled)

69. The SBP of claim 4, wherein the SBP comprises or is combined with a material, wherein the material comprises a particle, wherein the particle comprises a nanoparticle.

70. (canceled)

71. The SBP of claim 4, wherein the SBP comprises or is combined with a material, wherein the material comprises a coating, wherein the coating comprises a coating agent.

72. The SBP of claim 71, wherein the coating agent is selected from the group consisting of processed silk, paints, lacquers, adhesives, surfactants, particles, liquids, metals, lipids, oils, proteins, plastics, polymers, insulations, films, membranes, polyethylene glycol, methylcellulose, hypromellose, ethylcellulose, gelatin, hydroxypropyl cellulose, titanium dioxide, zein, poly(alkyl)(meth)acrylate, and/or poly(ethylene-co-vinyl acetate and any of the excipients listed in Table 1.

73. (canceled)

74. The SBP of claim 4, wherein the material comprises at least one excipient, and wherein the at least one excipient comprises one or more members selected from the group consisting of:
a. a lipid, lipid nanoparticle, and/or liposome, wherein the lipid, lipid nanoparticle, and/or liposome comprises one or more members selected from the group consisting of any of those listed in Table 1;
b. a bulking agent, wherein the bulking agent comprises one or more members selected from the group consisting of any of those listed in Table 1;
c. a sweetener, wherein the sweetener comprises one or more members selected from the group consisting of any of those listed in Table 1;
d. a colorant, wherein the colorant comprises one or more members selected from the group consisting of any of those listed in Table 1;
e. a preservative, wherein the preservative comprises one or more members selected from the group consisting of any of those listed in Table 1;
f. a flowability agent, wherein the flowability agent comprises one or more members selected from the group consisting of any of those listed in Table 1; and
g. a compound or composition selected from one or more members of the group consisting of any of those listed in Table 1.

75. The SBP of claim 4, wherein the SBP comprises or is combined with a material, wherein the material comprises a plastic, a plastic replacement, a polyolefin, a fabric, an electronic, a device, and/or a food product.

76. A method of preparing a SBP for use in a therapeutic application, an agricultural application, and/or a material science application, wherein the SBP comprises processed silk, the method comprising:
a. preparing the processed silk, wherein the processed silk comprises or is derived from one or more articles selected from the group consisting of raw silk, silk fiber, silk fibroin, and a silk fibroin fragment; and
b. preparing the SBP using the processed silk.

77. The method of claim 76, wherein preparing the processed silk comprises one or more methods selected from the group consisting of:
a. harvesting raw silk from a silk producer, wherein the silk producer comprises a wild type organism or a genetically modified organism;
b. degumming raw silk and/or silk fiber comprising treating the raw silk and/or silk fiber with degumming solution, wherein the degumming solution comprises at least one degumming agent comprising one or more members selected from the group consisting of water, alcohols, soaps, acids, alkaline solutions, detergents, salts, and enzymes;
c. preparing a processed silk solution, wherein the processed silk solution includes silk fibroin and a solvent, wherein the solvent comprises one or more members selected from the group consisting of an organic solvent, water, saline, high salt solution, and buffer;
d. purifying and/or concentrating silk fibroin;
e. drying processed silk, wherein drying is carried out according to a method comprising one or more members selected from the group consisting of oven drying, lyophilizing, and air drying; and
f. preparing a processed silk format:
i. wherein the processed silk format comprises one or more formats selected from the group consisting of adhesives, capsules, coatings, cocoons, combs, cones, cylinders, discs, emulsions, fibers, films, foams, gels, grafts, hydrogels, implants, mats, membranes, microspheres, nanofibers, nanoparticles, nanospheres, nets, organogels, particles, patches, powders, rods, scaffolds, sheets, solids, solutions, sponges, sprays, spuns, suspensions, tablets, threads, tubes, vapors, and yarns; and
ii. wherein the processed silk format is prepared by a process comprising one or more members selected from the group consisting of acidifying, air drying, alkalinizing, annealing, chemical crosslinking, chemical modification, concentration, cross-linking, degumming, dissolving, dry spinning, drying, electrifying, electrospinning, electrospraying, emulsifying, encapsulating, extraction, extrusion, gelation, harvesting, heating, lyophilization, molding, oven drying, pH alteration, precipitation, purification, shearing, sonication, spinning, spray drying, spray freezing, spraying, vapor annealing, vortexing, and water annealing.

78-83. (canceled)

84. The method of claim 77, wherein preparing the processed silk comprises degumming raw silk and/or silk fiber in degumming solution, wherein the raw silk and/or silk fiber are heated in the degumming solution.

85. The method of claim 84, wherein the raw silk and/or silk fiber are heated in the degumming solution at a temperature of from about 4° C. to about 115° C.

86. The method of claim 85, wherein the raw silk and/or silk fiber are heated in degumming solution for a period of from about 10 seconds to about 24 hours.

87-110. (canceled)

111. A method of: (1) treating, preventing, mitigating, alleviating, curing, and/or diagnosing a disease, disorder, and/or condition in a subject; (2) restoring or promoting health, nutrition and/or wellbeing of a subject; and/or (3) supporting or promoting reproduction in a subject, the method comprising contacting the subject with the SBP of claim 1.

112. (canceled)

113. The method of claim 111, wherein the SBP is administered to the subject by a route of administration selected from the group consisting of auricular administration, intraarticular administration, intramuscular administration, intrathecal administration, extracorporeal administration, buccal administration, intrabronchial administration, conjunctival administration, cutaneous administration, dental administration, endocervical administration, endosinusial administration, endotracheal administration, enteral administration, epidural administration, intra-abdominal administration, intrabiliary administration, intrabursal administration, oropharyngeal administration, interstitial administration, intracardiac administration, intracartilaginous administration, intracaudal administration, intracavernous administration, intracerebral administration, intracorporous cavernosum, intracavitary administration, intracorneal administration, intracisternal administration, cranial administration, intracranial administration, intradermal administration, intralesional administration, intratympanic administration, intragingival administration, intraovarian administration, intraocular administration, intradiscal administration, intraductal administration, intraduodenal administration, ophthalmic administration, intradural administration, intraepidermal administration, intraesophageal administration, nasogastric administration, nasal administration, laryngeal administration, intraventricular administration, intragastric administration, intrahepatic administration, intraluminal administration, intravitreal administration, intravesicular administration, intralymphatic administration, intramammary administration, intramedullary administration, intrasinal administration, intrameningeal administration, intranodal administration, intraovarian administration, intrapulmonary administration, intrapericardial administration, intraperitoneal administration, intrapleural administration, intrapericardial administration, intraprostatic administration, intrapulmonary administration, intraluminal administration, intraspinal administration, intrasynovial administration, intratendinous administration, intratesticular administration, subconjunctival administration, intracerebroventricular administration, epicutaneous administration, intravenous administration, retrobulbar administration, periarticular administration, intrathoracic administration, subarachnoid administration, intratubular administration, periodontal administration, transtympanic administration, transtracheal administration, intratumor administration, vaginal administration, urethral administration, intrauterine administration, oral administration, gastroenteral administration, parenteral administration, sublingual administration, ureteral administration, percutaneous administration, peridural administration, transmucosal administration, perineural administration, transdermal administration, rectal administration, soft tissue administration, intraarterial administration, subcutaneous administration, topical administration, extra-amniotic administration, insufflation, enema, eye drops, ear drops, and intravesical infusion.

114-115. (canceled)


SEQUENCE LISTING (1 sequence)

SEQ ID NO: 1 - protein, 6 aa, Artificial Sequence
  Description of Artificial Sequence Synthetic Sequence
GAGAGS